(19) United States
(12) Patent Application Publication
(10) Pub. No.: US 2026/0209231 A1
(43) Pub. Date: Jul. 23, 2026

(54) BIFUNCTIONAL SELECTIVE DEGRADERS OF SMARCA2 AND THERAPEUTIC USES THEREOF

(71) Applicant: Nurix Therapeutics, Inc., San Francisco, CA (US)

(72) Inventors: Thiago Augusto GRIGOLO, San Francisco, CA (US); Rishab N. IYER, San Francisco, CA (US); Tim R. KANE, San Francisco, CA (US); Katherine KEENAN, San Francisco, CA (US); Mike E. LIZARZABURU, San Francisco, CA (US); Jake C. SWARTZEL, San Francisco, CA (US); Alexander E. VALDES, San Francisco, CA (US)

(21) Appl. No.: 19/416,581

(22) Filed: Dec. 11, 2025

Related U.S. Application Data
(60) Provisional application No. 63/730,921, filed on Dec. 11, 2024.

(51) Int. Cl. C07D 471/10; A61K 31/501; A61K 31/506; A61K 31/513; A61K 31/5386; C07D 401/14; C07D 413/14; C07D 417/14; C07D 451/02; C07D 487/08; C07D 491/107; C07D 498/10; C07D 519/00

(52) U.S. Cl. CPC C07D 471/10; A61K 31/501; A61K 31/506; A61K 31/513; A61K 31/5386; C07D 401/14; C07D 413/14; C07D 417/14; C07D 451/02; C07D 487/08; C07D 491/107; C07D 498/10; C07D 519/00

(57) ABSTRACT

The present disclosure provides bifunctional compounds as selective SMARCA2 degraders via ubiquitin proteosome pathway, and their therapeutic use for treating cancers.

### CROSS-REFERENCE TO RELATED APPLICATIONS

This application claims the benefit of priority to U.S. Provisional Application No. 63/730,921, filed Dec. 11, 2024, which application is hereby incorporated by reference in its entirety.

### BACKGROUND

### Technical Field

The present invention provides novel bifunctional compounds for proteolytically degrading SMARCA2, and their therapeutic use for treating SMARCA2-mediated diseases, including cancers.

### Description of the Related Art

Mammalian SWItch/Sucrose Non-Fermentable (mSWI/SNF) chromatin remodeling complexes contain one of two mutually exclusive and highly homologous catalytic ATPase subunits, SMARCA2 or SMARCA4. Subunit mutations within the complexes are prevalent in cancer, and SMARCA4-mutated cancers show acute sensitivity to depletion of SMARCA2, making SMARCA2 an attractive oncological target.

Several groups have developed small molecule inhibitors of SMARCA2, but advancement of these molecules has been hindered by the requirement for selectivity over SMARCA4. See e.g., Wanior, M. et al J. Med. Chem. 63:14680 (2020). Kofink, C. et al Nature Commun. 13:5969 (2022). Two regions of the SMARCA2 protein with well-defined binding pockets relevant for small molecule inhibitor discovery are the catalytic ATPase domain and a conserved bromodomain that can interact with acetylated chromatin. High sequence homology between the ATPase domains of these proteins makes it challenging to develop inhibitors with sufficient selectivity. Known ATPase domain ligands are dual inhibitors of SMARCA2/4 and exhibit dose-limiting tolerability issues. Alternatively, while the bromodomain shows more potential for selectivity, its function is dispensable, and current ligands are ineffective at inhibiting cell proliferation.

While selective targeting of SMARCA2 over SMARCA4 has thus far proven challenging using inhibitors, repurposing the non-inhibitory bromodomain binders for use as targeted protein degraders holds promise. However, current degraders have shown poor selectivity in targeting SMARCA2 over SMARCA4. See e.g., WO 2021/083949, WO 2020/251971 and WO 2019/195201.

### BRIEF SUMMARY

Provided herein are bifunctional compounds exhibiting both potent inhibition and selectivity against SMARCA2 while sparing SMARCA4.

Provided herein are bifunctional compounds represented by Formula (I):

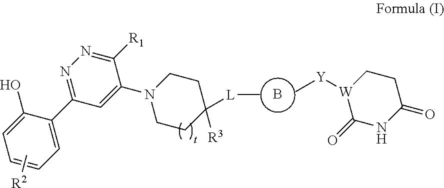

or a stereoisomer, pharmaceutically acceptable salt thereof, wherein:
t is 0 or 1;
R¹ is hydrogen or C₁₋₆ alkyl optionally substituted with 1 to 3 R^{a};
R² is hydrogen, halo, hydroxyl, —O—R^{c}, C₁₋₆ alkyl optionally substituted with 1 to 3 R^{a};
R³ is hydrogen, —CN, C₁₋₆ alkyl, C₆₋₁₂ aryl, C₃₋₁₂ cycloalkyl, 5-12 membered heteroaryl, each being optionally substituted with 1 to 3 R^{d}, or —O—R^{e};
L comprises up to 8 linker segments represented by -L₁-L₂-L₃-L₄-L₅-L₆-L₇-L₈-, each L₁, L₂, L₃, L₄, L₅, L₆, L₇, or L₈, being independently:
   i) C₃₋₁₂ cycloalkyl optionally substituted with 1-3 R^{b};
   ii) C₆₋₁₂ aryl optionally substituted with 1-3 R^{b};
   iii) 4-12 membered heterocyclyl optionally substituted with 1-3 R^{b};
   iv) 5-12 membered heteroaryl optionally substituted with 1-3 R^{b};
   v) direct bond;
   vi) C₁₋₁₂ alkylene chain optionally substituted with 1-3 R^{b}; or
   vii) —(CH₂)ₘ—C(O)—, —(CH₂)ₘ—C(O)O—, —(CH₂)ₘ—O—, —(CH₂)ₘ—N(R^{c})—, —(CH₂)ₘ—S—, —(CH₂)ₘ—C(S)—, —(CH₂)ₘ—C(S)—O—, —(CH₂)ₘ—S(O)₂—, —(CH₂)ₘ—S(O)═N—, —(CH₂)ₘ—S(O)₂NH—, —(CH₂)ₘ—C(O)—N(R^{c})—, —C(O)—N(R^{c})—(CH₂)ₘ—, —(CH₂)ₘ—O—C(O)—N(R^{c})—, —(CH₂)ₘ—O—C(O)—O—, or —NH—(CH₂)ₘ—C(O)—, wherein m is 0, 1, 2, 3, 4, 5 or 6;
each R^{a} is independently halo, or —O—R;
each R^{b} is independently oxo, imino, sulfoximino, halo, nitro, —CN, C₁₋₆ alkyl, C₂₋₆ alkenyl, C₃₋₁₅ cycloalkyl, C₁₋₈ haloalkyl, C₆₋₁₂ aryl, 5-12 membered heteroaryl, 4-12 membered heterocyclyl, —O—R^{c}, —C(O)—R^{c}, —C(O)O—R^{c}, —C(O)—N(R^{c})(R^{c}), —N(R^{c})(R^{c}), —N(R^{c})C(O)—R^{c}, —N(R^{c})C(O)O—R^{c}, —N(R^{c})C(O)N(R^{c})(R^{c}), —N(R^{c})S(O)₂(R^{c}), —NR^{c}S(O)₂N(R^{c})(R^{c}), —N(R^{c})S(O)₂O(R^{c}), —OC(O)R^{c}, —OC(O)—N(R^{c})(R^{c}), —Si(R^{c})₃, —S—R^{c}, —S(O)R^{c}, —S(O)(NH)R^{c}, —S(O)₂R^{c} or —S(O)₂N(R^{c})(R^{c}), wherein each of C₁₋₆ alkyl, C₂₋₆ alkenyl, C₃₋₁₅ cycloalkyl, C₁₋₈ haloalkyl, C₆₋₁₂ aryl, 5-12 membered heteroaryl, and 4-12 membered heterocyclyl may be optionally substituted with 1 to 3 R^{d};
each R^{c} is independently hydrogen, C₁₋₆ alkyl, C₁₋₆ alkoxy, C₃₋₆ cycloalkyl, C₆₋₁₂ aryl, or C₁₋₆ haloalkyl, wherein C₁₋₆ alkyl is optionally substituted with one or more substituents selected from C₁₋₃ alkoxy, hydroxy, C₃₋₆ cycloalkyl, C₆₋₁₂ aryl and amino;
each R^{d} is independently halo, —CN, —O—R^{c}, C₁₋₆ alkyl, C₆₋₁₂ aryl or C₁₋₆ haloalkyl;
R^{e} is C₁₋₆ alkyl, C₃₋₁₂ cycloalkyl, C₆₋₁₂ aryl, 5-12 membered heteroaryl, each being optionally substituted with C₁₋₆ alkyl, C₁₋₆ haloalkyl or halo;
W is —C(R^{g})— or —N—;
Y is direct bond, C₁₋₄ alkylene chain, —C(O)—, —C(O)O—, —O—, —N(R^{g})—, —S— —C(S)—, —C(S)—O—, —O—C(O)O—, —C(O)—N(R^{g})—, or —O—C(O)—N(R^{g})—;
B ring is C₆₋₁₂ aryl, 5-12 membered heteroaryl, or 4-12 membered heterocyclyl, each being optionally substituted with 1 to 3 R^{j};
R^{g} is hydrogen or C₁₋₆ alkyl; and
each R^{j} is independently halo, oxo, —CN, —O—R^{c}, C₁₋₆ alkyl, or C₁₋₆ haloalkyl.

Also provided herein are compounds having the structure represented by Formula (II):

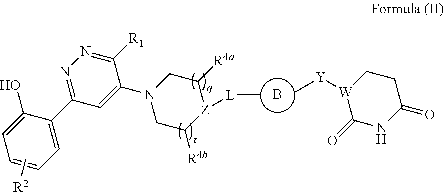

or a stereoisomer, pharmaceutically acceptable salt thereof, wherein:
t and q is independently 0 or 1;
Z is —N— or —C(R³)—;
R¹ is hydrogen or C₁₋₆ alkyl optionally substituted with 1 to 3 R^{a};
R² is hydrogen, halo, hydroxyl, —O—R^{c}, C₁₋₆ alkyl optionally substituted with 1 to 3 R^{a};
R³ is hydrogen, —CN, halo (preferably F), —O—R^{e}, C₁₋₆ alkyl, C₆₋₁₂ aryl, C₃₋₁₂ cycloalkyl, 5-12 membered heteroaryl, wherein each of C₁₋₆ alkyl, C₆₋₁₂ aryl, C₃₋₁₂ cycloalkyl, and 5-12 membered heteroaryl is optionally substituted with 1 to 3 R^{d};
R^{4a} and R^{4b} are each independently hydrogen, C₁₋₆ alkyl or C₁₋₆ alkoxy, or
R^{4a} and R^{4b} together may form a C₁₋₃ alkylene chain; or
R^{4a} and R³ together with the carbons to which they are attached form a C₃₋₁₂ cycloalkyl or 5-12 membered heterocycle,
L comprises up to 8 linker segments represented by -L₁-L₂-L₃-L₄-L₅-L₆-L₇-L₈-, each L₁, L₂, L₃, L₄, L₅, L₆, L₇, or L₈, being independently:
   i) C₃₋₁₂ cycloalkyl optionally substituted with 1-3 R^{b};
   ii) C₆₋₁₂ aryl optionally substituted with 1-3 R^{b};
   iii) 4-12 membered heterocyclyl optionally substituted with 1-3 R^{b};
   iv) 5-12 membered heteroaryl optionally substituted with 1-3 R^{b};
   v) direct bond;
   vi) C₁₋₁₂ alkylene chain optionally substituted with 1-3 R^{b};
   vii) C₁₋₁₂ alkenylene chain optionally substituted with 1-3 R^{b}; or
viii) —(CH₂)ₘ—C(O)—, —(CH₂)ₘ—C(O)O—, —(CH₂)ₘ—O—, —(CH₂)ₘ—N(R^{c})—, —(CH₂)ₘ—S—, —(CH₂)ₘ—C(S)—, —(CH₂)ₘ—C(S)—O—, —(CH₂)ₘ—S(O)₂—, —(CH₂)ₘ—S(O)=N—, —(CH₂)ₘ—S(O)₂NH—, —(CH₂)ₘ—C(O)—N(R^{c})—, —C(O)—N(R^{c})—(CH₂)ₘ—, —(CH₂)ₘ—O—C(O)—N(R^{c})—, —(CH₂)ₘ—O—C(O)—O—, or —NH—(CH₂)ₘ—C(O)—, wherein m is 0, 1, 2, 3, 4, 5 or 6;
each R^{a} is independently halo, or —O—R;
each R^{b} is independently oxo, imino, sulfoximino, halo, nitro, —CN, C₁₋₆ alkyl, C₂₋₆ alkenyl, C₃₋₁₅ cycloalkyl, C₁₋₈ haloalkyl, C₆₋₁₂ aryl, 5-12 membered heteroaryl, 4-12 membered heterocyclyl, —O—R^{c}, —C(O)—R^{c}, —C(O)O—R^{c}, —C(O)—N(R^{c})(R^{c}), —N(R^{c})(R^{c}), —N(R^{c})C(O)—R^{c}, —N(R^{c})C(O)O—R^{c}, —N(R^{c})C(O)N(R^{c})(R^{c}), —N(R^{c})S(O)₂(R^{c}), —NR^{c}S(O)₂N(R^{c})(R^{c}), —N(R^{c})S(O)₂O(R^{c}), —OC(O)R^{c}, —OC(O)—N(R^{c})(R^{c}), —Si(R^{c})₃, —S—R^{c}, —S(O)R^{c}, —S(O)(NH)R^{c}, —S(O)₂R^{c} or —S(O)₂N(R^{c})(R^{c}), wherein each of C₁₋₆ alkyl, C₂₋₆ alkenyl, C₃₋₁₅ cycloalkyl, C₁₋₈ haloalkyl, C₆₋₁₂ aryl, 5-12 membered heteroaryl, and 4-12 membered heterocyclyl may be optionally substituted with 1 to 3 R^{d};
each R^{c} is independently hydrogen, C₁₋₆ alkyl, C₁₋₆ alkoxy, C₃₋₆ cycloalkyl (optionally substituted with 1 or 2 halo, C₁₋₃ alkyl or C₁₋₃ alkoxy), C₆₋₁₂ aryl, 4-12 membered heterocyclyl optionally substituted with 1 or 2 C₁₋₃ alkyl), 5-12 membered heteroaryl optionally substituted with 1 or 2 C₁₋₃ alkyl or C₁₋₃ haloalkyl) or C₁₋₆ haloalkyl, wherein C₁₋₆ alkyl is optionally substituted with one or more substituents selected from C₁₋₃ alkoxy, hydroxy, C₃₋₆ cycloalkyl (optionally further substituted with 1 or 2 halo, C₁₋₃ alkoxy or C₁₋₃ alkyl), CN, 5-12 membered heteroaryl (optionally further substituted with 1 or 2 halo or C₁₋₃ alkyl), C₆₋₁₂ aryl and amino;
each R^{d} is independently halo, —CN, —O—R^{c}, C₁₋₆ alkyl, C₆₋₁₂ aryl, C₃₋₆ cycloalkyl or C₁₋₆ haloalkyl;
R^{e} is C₁₋₆ alkyl, C₃₋₁₂ cycloalkyl, C₆₋₁₂ aryl, 5-12 membered heteroaryl, each being optionally substituted with C₁₋₆ alkyl, C₁₋₆ alkoxy, C₁₋₆ haloalkyl or halo;
W is —C(R^{g})— or —N—;
Y is direct bond, C₁₋₄ alkylene chain, —C(O)—, —C(O)O—, —O—, —N(R^{g})—, —S— —C(S)—, —C(S)—O—, —O—C(O)O—, —C(O)—N(R^{g})—, or —O—C(O)—N(R^{g})—;
B ring is C₆₋₁₂ aryl, 5-12 membered heteroaryl, or 4-12 membered heterocyclyl, each being optionally substituted with 1 to 3 R^{j};
R^{g} is hydrogen or C₁₋₆ alkyl; and
each R^{j} is independently halo, oxo, —CN, —O—R^{c}, C₁₋₆ alkyl, or C₁₋₆ haloalkyl.

In further more specific embodiments, t is 1, and the compound of Formula (I) has the following structure:

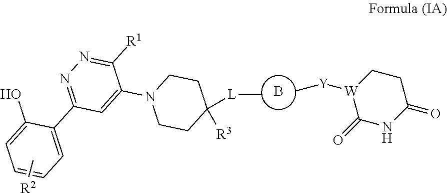

In other more specific embodiments, t is 0, and the compound of Formula (I) has the following structure:

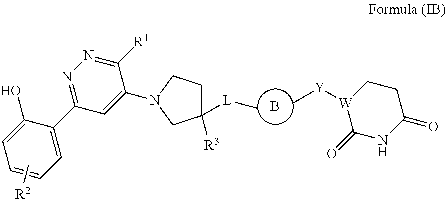

In various other more specific embodiments, compounds of Formula (II) have one of the following structures:

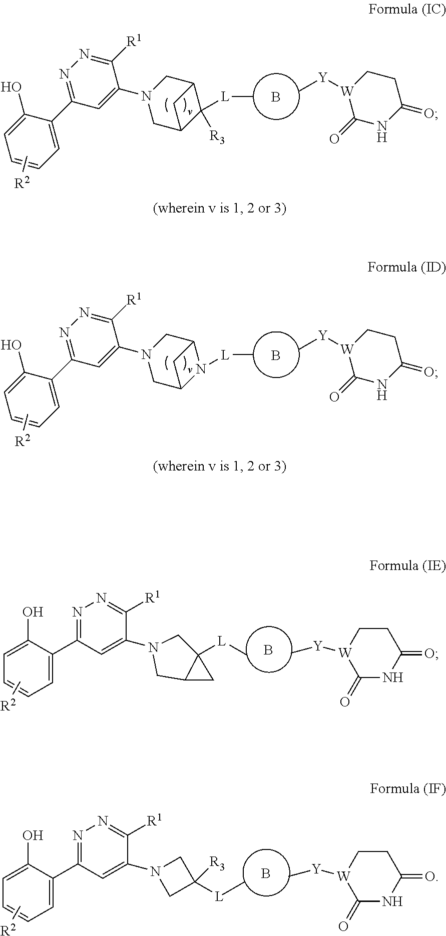

In various more specific embodiments, each L₁, L₂, L₃, L₄, L₅, L₆, L₇, or L₈ is independently:
i) a bivalent ring moiety selected from the group consisting of:

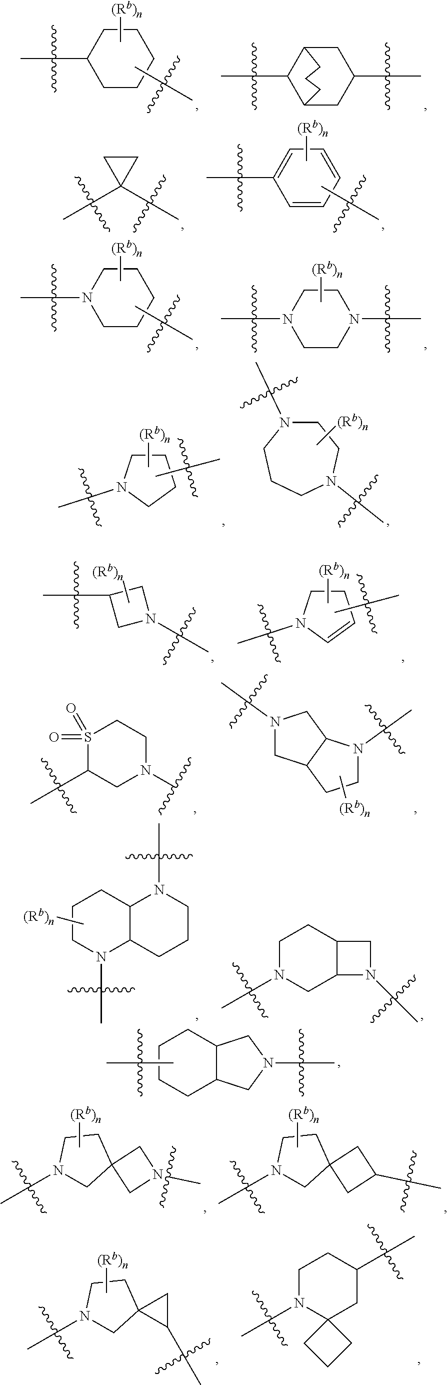

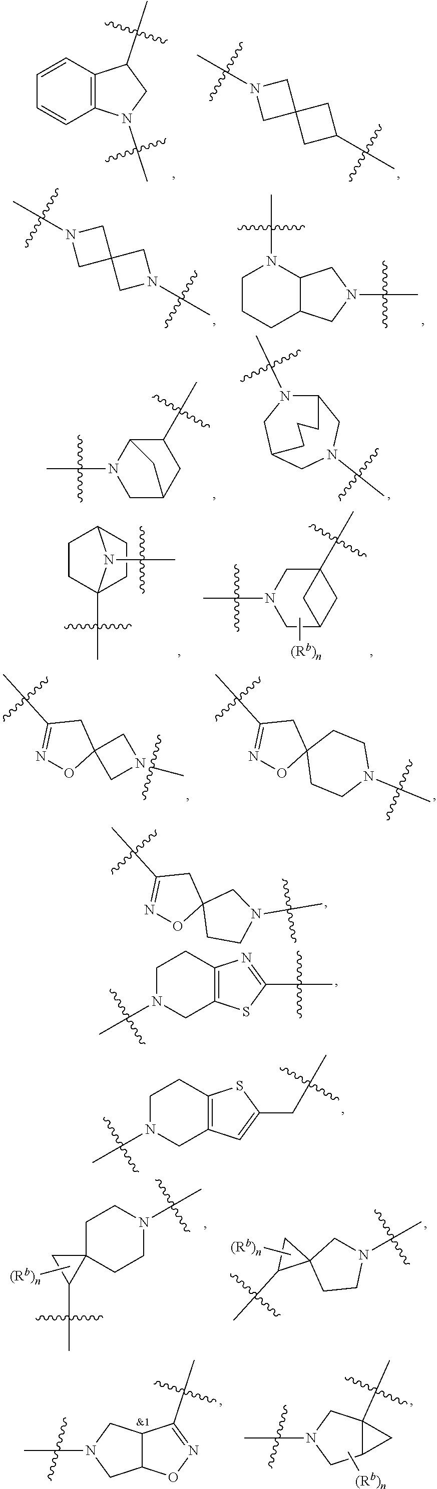

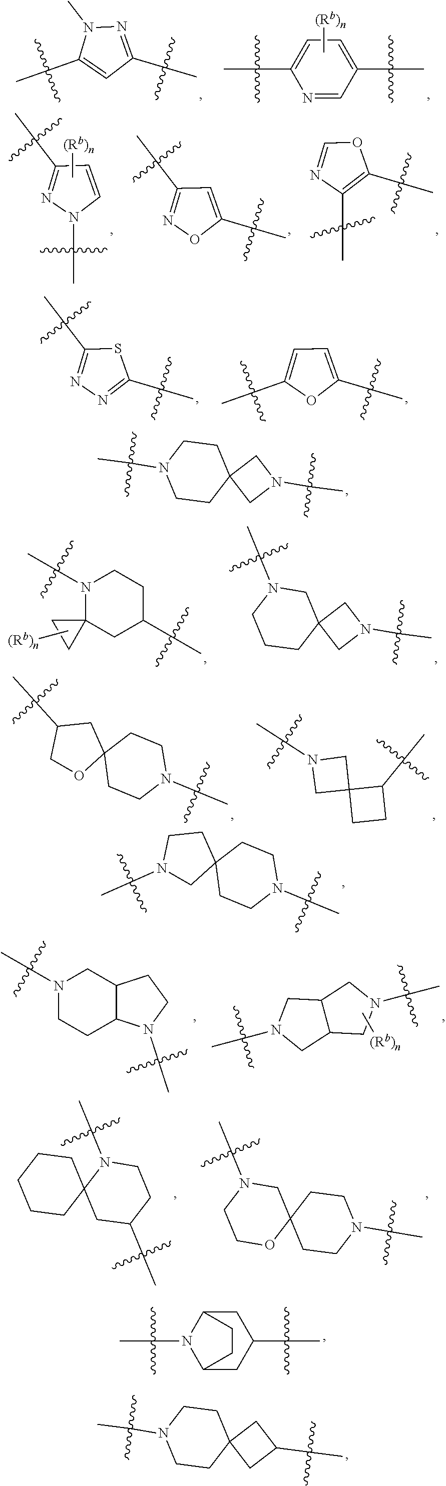

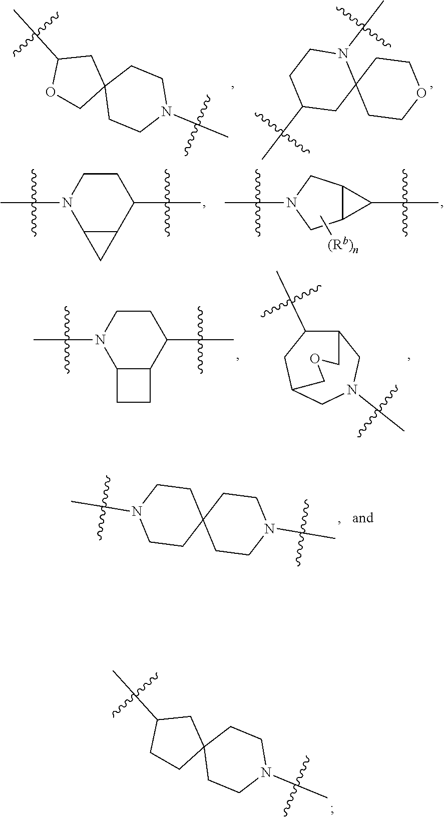

ii) direct bond;
iii) C₁₋₆ alkylene chain optionally substituted with 1-3 R^{b}; or
iv) —C(O)—, —O—, —(CH₂)ₘ—C(O)—N(R^{c})—, —(CH₂)ₘ—C(O)—, —(CH₂)ₘ—N(R^{c})—, —C(O)—N(R^{c})—(CH₂)ₘ, or —NH—(CH₂)ₘ—C(O)—, —(CH₂)ₘ—S(O)₂NH—, m being 0, 1, 2 or 3; wherein,
n is 0, 1, or 2;
R^{b} is halo, —O—R^{c}, —CN, C₁₋₆ alkyl, or C₁₋₆ haloalkyl; and
R^{c} is hydrogen, C₁₋₆ alkyl, C₁₋₆ alkoxy, C₃₋₆ cycloalkyl, phenyl, or C₁₋₆ haloalkyl, wherein C₁₋₆ alkyl is optionally substituted with C₁₋₃ alkoxy, hydroxy, C₃₋₆ cycloalkyl, phenyl and amino.

In various more specific embodiments, Y is direct bond, —C(O)—N(R^{g})— (wherein R^{g} is hydrogen or C₁₋₆ alkyl), or —NH—; and
B ring is:

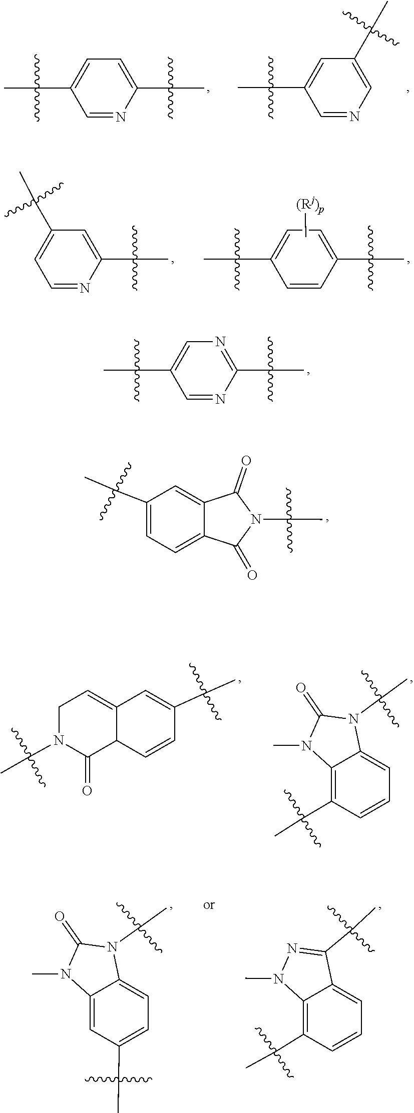

wherein p is 0, 1 or 2, R^{j} is halo, —CN, —O—R^{c}, C₁₋₆ alkyl, or C₁₋₆ haloalkyl.

Also provided herein are compounds of the following substructures:

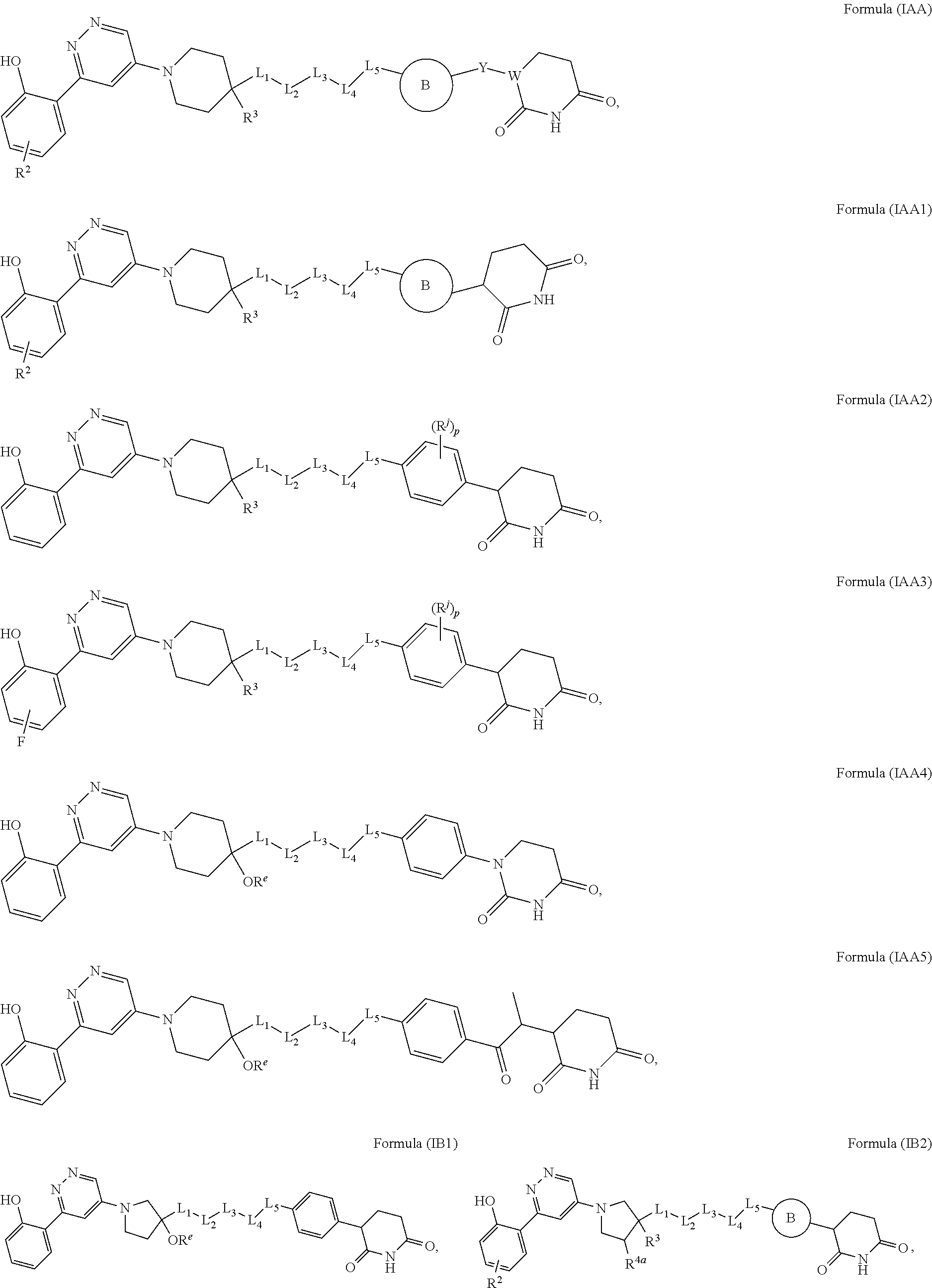

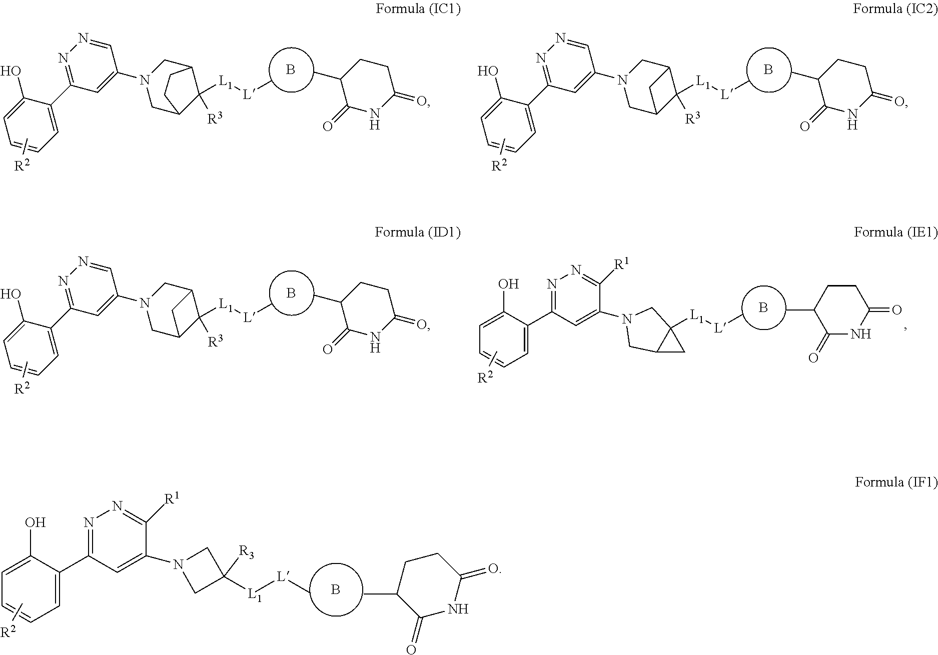

Also provided herein is a pharmaceutical composition comprising a compound of Formulae (I), (II) or any one of the substructures (IA), (IB), (IC), (ID), (IE), (IF), (IAA), (IAA1), (IAA2), (IAA3), (IAA4), (IAA5), (IB1), (IB2), (IC1), (IC2), (ID1), (IE1), or (IF1) or any one of the specific compounds of Examples 1-263 or a pharmaceutically acceptable salt thereof, and a pharmaceutically acceptable excipient or carrier.

Further provided herein are pharmaceutical or therapeutic use of the compound and pharmaceutical composition described herein for treating SMARCA2 mediated diseases or disorders, including various types of cancers.

### DETAILED DESCRIPTION

Specific degradation of SMARCA2 could be accomplished by using heterobifunctional small molecules to recruit SMARCA2, with high selectivity over SMARCA4, to a ubiquitin ligase and thus promoting ubiquitylation and proteasomal degradation of SMARCA2. Thus, provided herein are bifunctional compounds, each comprising a selective SMARCA2 binder, a covalent a linker moiety (L) and a ligase harness moiety (LHM) for targeting ubiquitin ligase. Preferably, the LHM targets cereblon (CRBN) proteins, which are substrate recognition subunits of two ubiquitously expressed and biologically important Cullin RING E3 ubiquitin ligase complexes. See, e.g., WO 2019/099926, WO 2020/023851, and U.S. Published Application No. 2019/0192668.

Various embodiments provide compounds having a structure represented by Formula (I):

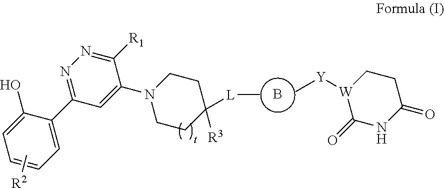

or a stereoisomer, pharmaceutically acceptable salt thereof, wherein:
t is 0 or 1;
R¹ is hydrogen or C₁₋₆ alkyl optionally substituted with 1 to 3 R^{a};
R² is hydrogen, halo, hydroxyl, —O—R^{c}, C₁₋₆ alkyl optionally substituted with 1 to 3 R^{a};
R³ is hydrogen, —CN, C₁₋₆ alkyl, C₆₋₁₂ aryl, C₃₋₁₂ cycloalkyl, 5-12 membered heteroaryl, each being optionally substituted with 1 to 3 R^{d}, or —O—R^{e};
L comprises up to 8 linker segments represented by -L₁-L₂-L₃-L₄-L₅-L₆-L₇-L₈-, each L₁, L₂, L₃, L₄, L₅, L₆, L₇, or L₈, being independently:
   i) C₃₋₁₂ cycloalkyl optionally substituted with 1-3 R^{b};
   ii) C₆₋₁₂ aryl optionally substituted with 1-3 R^{b};
   iii) 4-12 membered heterocyclyl optionally substituted with 1-3 R^{b};
   iv) 5-12 membered heteroaryl optionally substituted with 1-3 R^{b};
   v) direct bond;
   vi) C₁₋₁₂ alkylene chain optionally substituted with 1-3 R^{b}; or
   vii) —(CH₂)ₘ—C(O)—, —(CH₂)ₘ—C(O)O—, —(CH₂)ₘ—O—, —(CH₂)ₘ—N(R^{c})—, —(CH₂)ₘ—S—, —(CH₂)ₘ—C(S)—, —(CH₂)ₘ—C(S)—O—, —(CH₂)ₘ—S(O)₂—, —(CH₂)ₘ—S(O)=N—, —(CH₂)ₘ—S(O)₂NH—, —(CH₂)ₘ—C(O)—N(R^{c})—, —C(O)—N(R^{c})—(CH₂)ₘ—, —(CH₂)ₘ—O—C(O)—N(R^{c})—, —(CH₂)ₘ—O—C(O)—O—, or —NH—(CH₂)ₘ—C(O)—, wherein m is 0, 1, 2, 3, 4, 5 or 6;
each R^{a} is independently halo, or —O—R^{c};
each R^{b} is independently oxo, imino, sulfoximino, halo, nitro, —CN, C₁₋₆ alkyl, C₂₋₆ alkenyl, C₃₋₁₅ cycloalkyl, C₁₋₈ haloalkyl, C₆₋₁₂ aryl, 5-12 membered heteroaryl, 4-12 membered heterocyclyl, —O—R^{c}, —C(O)—R^{c}, —C(O)O—R^{c}, —C(O)—N(R^{c})(R^{c}), —N(R^{c})(R^{c}), —N(R^{c})C(O)—R^{c}, —N(R^{c})C(O)O—R^{c}, —N(R^{c})C(O)N(R^{c})(R^{c}), —N(R^{c})S(O)₂(R^{c}), —NR^{c}S(O)₂N(R^{c})(R^{c}), —N(R^{c})S(O)₂O(R^{c}), —OC(O)R^{c}, —OC(O)—N(R^{c})(R^{c}), —Si(R^{c})₃, —S—R^{c}, —S(O)R^{c}, —S(O)(NH)R^{c}, —S(O)₂R^{c} or —S(O)₂N(R^{c})(R^{c}), wherein each of C₁₋₆ alkyl, C₂₋₆ alkenyl, C₃₋₁₅ cycloalkyl, C₁₋₈ haloalkyl, C₆₋₁₂ aryl, 5-12 membered heteroaryl, and 4-12 membered heterocyclyl may be optionally substituted with 1 to 3 R^{d};
each R^{c} is independently hydrogen, C₁₋₆ alkyl, C₁₋₆ alkoxy, C₃₋₆ cycloalkyl, C₆₋₁₂ aryl, or C₁₋₆ haloalkyl, wherein C₁₋₆ alkyl is optionally substituted with one or more substituents selected from C₁₋₃ alkoxy, hydroxy, C₃₋₆ cycloalkyl, C₆₋₁₂ aryl and amino;
each R^{d} is independently halo, —CN, —O—R^{c}, C₁₋₆ alkyl, C₆₋₁₂ aryl or C₁₋₆ haloalkyl;
R^{e} is C₁₋₆ alkyl, C₃₋₁₂ cycloalkyl, C₆₋₁₂ aryl, 5-12 membered heteroaryl, each being optionally substituted with C₁₋₆ alkyl, C₁₋₆ haloalkyl or halo;
W is —C(R^{g})— or —N—;
Y is direct bond, C₁₋₄ alkylene chain, —C(O)—, —C(O)O—, —O—, —N(R^{g})—, —S—, —C(S)—, —C(S)—O—, —O—C(O)O—, —C(O)—N(R^{g})—, or —O—C(O)—N(R^{g})—;
B ring is C₆₋₁₂ aryl, 5-12 membered heteroaryl, or 4-12 membered heterocyclyl, each being optionally substituted with 1 to 3 R^{j};
R^{g} is hydrogen or C₁₋₆ alkyl; and
each R^{j} is independently halo, oxo, —CN, —O—R^{c}, C₁₋₆ alkyl, or C₁₋₆ haloalkyl.

Also provides are compounds having a structure represented by Formula (II):

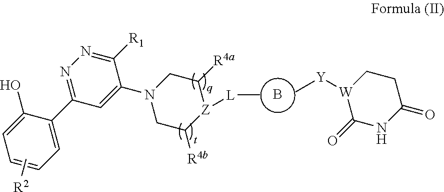

or a stereoisomer, pharmaceutically acceptable salt thereof, wherein:
t and q is independently 0 or 1;
Z is —N— or —C(R³)—;
R¹ is hydrogen or C₁₋₆ alkyl optionally substituted with 1 to 3 R^{a};
R² is hydrogen, halo, hydroxyl, —O—R^{c}, C₁₋₆ alkyl optionally substituted with 1 to 3 R^{a};
R³ is hydrogen, —CN, halo (preferably F), —O—R^{e}, C₁₋₆ alkyl, C₆₋₁₂ aryl, C₃₋₁₂ cycloalkyl, 5-12 membered heteroaryl, wherein each of C₁₋₆ alkyl, C₆₋₁₂ aryl, C₃₋₁₂ cycloalkyl, and 5-12 membered heteroaryl is optionally substituted with 1 to 3 R^{d};
R^{4a} and R^{4b} are each independently hydrogen, C₁₋₆ alkyl or C₁₋₆ alkoxy, or
R^{4a} and R^{4b} together may form a C₁₋₃ alkylene chain; or
R^{4a} and R³ together with the carbons to which they are attached form a C₃₋₁₂ cycloalkyl or 5-12 membered heterocycle,
L comprises up to 8 linker segments represented by -L₁-L₂-L₃-L₄-L₅-L₆-L₇-L₈-, each L₁, L₂, L₃, L₄, L₅, L₆, L₇, or L₈, being independently:
   i) C₃₋₁₂ cycloalkyl optionally substituted with 1-3 R^{b}.
   ii) C₆₋₁₂ aryl optionally substituted with 1-3 R^{b};
   iii) 4-12 membered heterocyclyl optionally substituted with 1-3 R^{b};
iv) 5-12 membered heteroaryl optionally substituted with 1-3 R^{b};
   v) direct bond;
   vi) C₁₋₁₂ alkylene chain optionally substituted with 1-3 R^{b};
   vii) C₁₋₁₂ alkenylene chain optionally substituted with 1-3 R^{b}; or
   viii) —(CH₂)ₘ—C(O)—, —(CH₂)ₘ—C(O)O—, —(CH₂)ₘ—O—, —(CH₂)ₘ—N(R^{c})—, —(CH₂)ₘ—S—, —(CH₂)ₘ—C(S)—, —(CH₂)ₘ—C(S)—O—, —(CH₂)ₘ—S(O)₂—, —(CH₂)ₘ—S(O)=N—, —(CH₂)ₘ—S(O)₂NH—, —(CH₂)ₘ—C(O)—N(R^{c})—, —C(O)—N(R^{c})—(CH₂)ₘ—, —(CH₂)ₘ—O—C(O)—N(R^{c})—, —(CH₂)ₘ—O—C(O)—O—, or —NH—(CH₂)ₘ—C(O)—, wherein m is 0, 1, 2, 3, 4, 5 or 6;
each R^{a} is independently halo, or —O—R^{c};
each R^{b} is independently oxo, imino, sulfoximino, halo, nitro, —CN, C₁₋₆ alkyl, C₂₋₆ alkenyl, C₃₋₁₅ cycloalkyl, C₁₋₈ haloalkyl, C₆₋₁₂ aryl, 5-12 membered heteroaryl, 4-12 membered heterocyclyl, —O—R^{c}, —C(O)—R^{c}, —C(O)O—R^{c}, —C(O)—N(R^{c})(R^{c}), —N(R^{c})(R^{c}), —N(R^{c})C(O)—R^{c}, —N(R^{c})C(O)O—R^{c}, —N(R^{c})C(O)N(R^{c})(R^{c}), —N(R^{c})S(O)₂(R^{c}), —NR^{c}S(O)₂N(R^{c})(R^{c}), —N(R^{c})S(O)₂O(R^{c}), —OC(O)R^{c}, —OC(O)—N(R^{c})(R^{c}), —Si(R^{c})₃, —S—R^{c}, —S(O)R^{c}, —S(O)(NH)R^{c}, —S(O)₂R^{c} or —S(O)₂N(R^{c})(R^{c}), wherein each of C₁₋₆ alkyl, C₂₋₆ alkenyl, C₃₋₁₅ cycloalkyl, C-s haloalkyl, C₆₋₁₂ aryl, 5-12 membered heteroaryl, and 4-12 membered heterocyclyl may be optionally substituted with 1 to 3 R^{d};
each R^{c} is independently hydrogen, C₁₋₆ alkyl, C₁₋₆ alkoxy, C₃₋₆ cycloalkyl (optionally substituted with 1 or 2 halo, C₁₋₃ alkyl or C₁₋₃ alkoxy), C₆₋₁₂ aryl, 4-12 membered heterocyclyl optionally substituted with 1 or 2 C₁₋₃ alkyl), 5-12 membered heteroaryl optionally substituted with 1 or 2 C₁₋₃ alkyl or C₁₋₃ haloalkyl) or C₁₋₆ haloalkyl, wherein C₁₋₆ alkyl is optionally substituted with one or more substituents selected from C₁₋₃ alkoxy, hydroxy, C₃₋₆ cycloalkyl (optionally further substituted with 1 or 2 halo, C₁₋₃ alkoxy or C₁₋₃ alkyl), CN, 5-12 membered heteroaryl (optionally further substituted with 1 or 2 halo or C₁₋₃ alkyl), C₆₋₁₂ aryl and amino;
each R^{d} is independently halo, —CN, —O—R^{c}, C₁₋₆ alkyl, C₆₋₁₂ aryl, C₃₋₆ cycloalkyl or C₁₋₆ haloalkyl;
R^{e} is C₁₋₆ alkyl, C₃₋₁₂ cycloalkyl, C₆₋₁₂ aryl, 5-12 membered heteroaryl, each being optionally substituted with C₁₋₆ alkyl, C₁₋₆ alkoxy, C₁₋₆ haloalkyl or halo;
W is —C(R^{g})— or —N—;
Y is direct bond, C₁₋₄ alkylene chain, —C(O)—, —C(O)O—, —O—, —N(R^{g})—, —S—, —C(S)—, —C(S)—O—, —O—C(O)O—, —C(O)—N(R^{g})—, or —O—C(O)—N(R^{g})—;
B ring is C₆₋₁₂ aryl, 5-12 membered heteroaryl, or 4-12 membered heterocyclyl, each being optionally substituted with 1 to 3 R^{j};
R^{g} is hydrogen or C₁₋₆ alkyl; and
each R^{j} is independently halo, oxo, —CN, —O—R^{c}, C₁₋₆ alkyl, or C₁₋₆ haloalkyl.

In more specific embodiments, t is 1, and the compound of Formula (I) has the following structure:

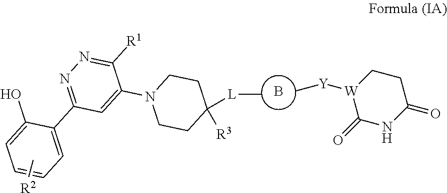

In other more specific embodiments, t is 0, and the compound of Formula (I) has the following structure:

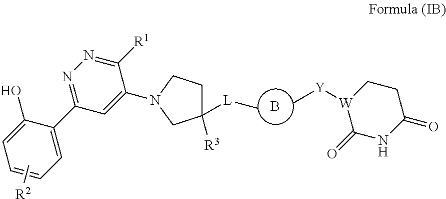

In other more specific embodiments, wherein t is 1, q is 1, R^{4a} and R^{4b} together form a C₁₋₃ alkylene chain, and the compound has one of the following structures:

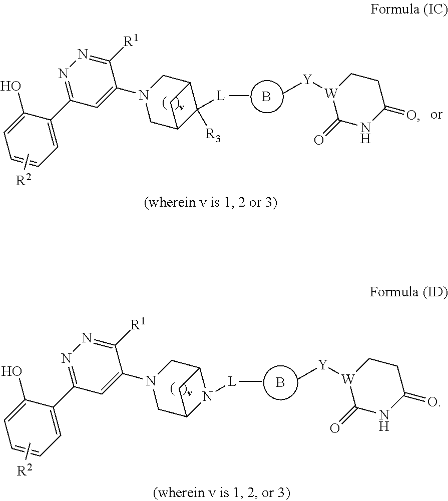

In other more specific embodiments, wherein t is 0, q is 1, and R^{4a} and R³ together form a methylene chain, and the compound has the following structure:

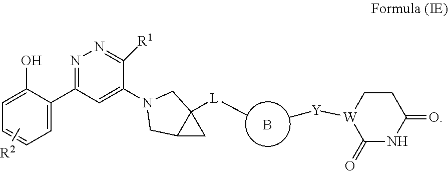

In other more specific embodiments, wherein t is 0, and q is 0, and the compound has the following structure:

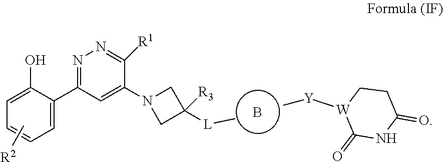

In various embodiments, the each L₁, L₂, L₃, L₄, L₅, L₆, L₇, or L₈ is independently:
i) a bivalent ring moiety selected from the group consisting of:

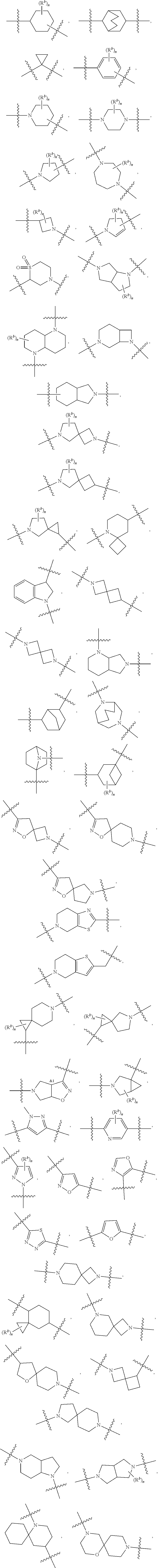

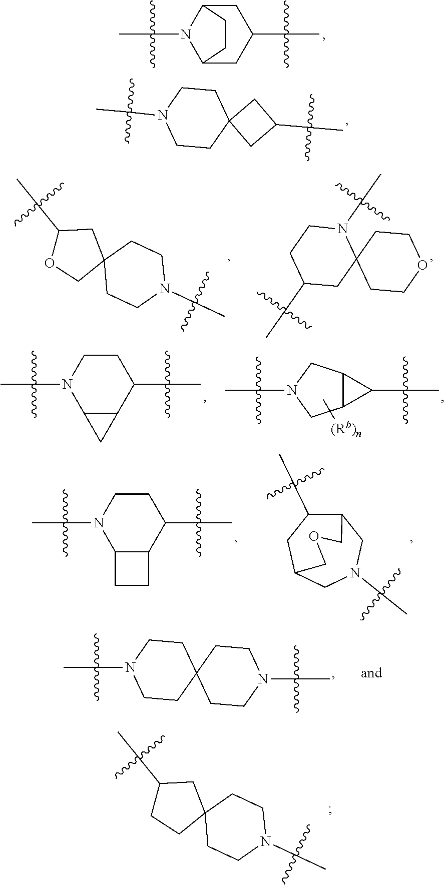

ii) direct bond;
iii) C₁₋₆ alkylene chain optionally substituted with 1-3 R^{b}; or
iv) —C(O)—, —O—, —(CH₂)ₘ—C(O)—N(R^{c})—, —(CH₂)ₘ—C(O)—, —(CH₂)ₘ—N(R^{c})—, —C(O)—N(R^{c})—(CH₂)ₘ—, or —NH—(CH₂)ₘ—C(O)—, —(CH₂)ₘ—S(O)₂NH—, m being 0, 1, 2 or 3;
   wherein,
n is 0, 1, or 2;
R^{b} is halo, —O—R^{c}, —CN, C₁₋₆ alkyl, or C₁₋₆ haloalkyl; and
R^{c} is hydrogen, C₁₋₆ alkyl, C₁₋₆ alkoxy, C₃₋₆ cycloalkyl, phenyl, or C₁₋₆ haloalkyl, wherein C₁₋₆ alkyl is optionally substituted with C₁₋₃ alkoxy, hydroxy, C₃₋₆ cycloalkyl, phenyl and amino.

In other more specific embodiments, Y is direct bond, —C(O)—N(R^{g})— (wherein Rg is hydrogen or C₁₋₆ alkyl), or —NH—; and
B ring is:

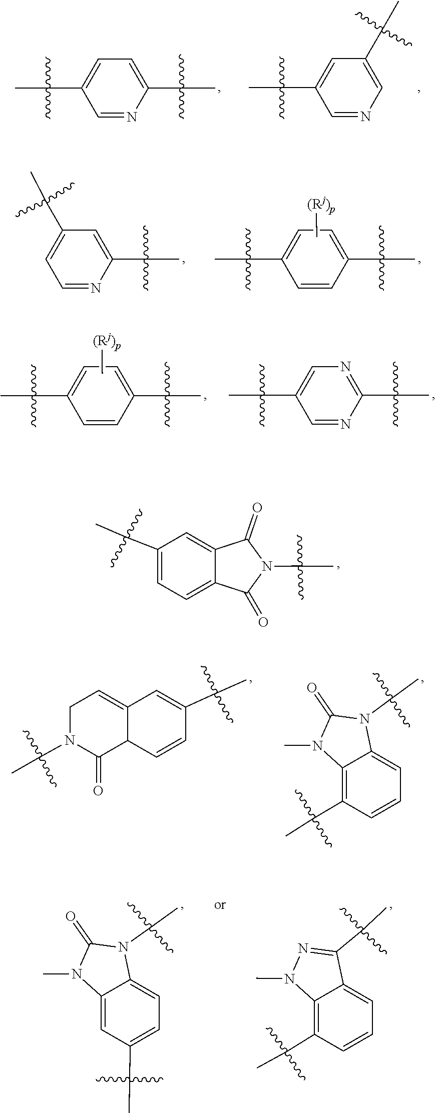

wherein p is 0, 1 or 2, R^{j} is halo, —CN, —O—R^{c}, C₁₋₆ alkyl, or C₁₋₆ haloalkyl.

In certain more specific embodiments, R¹ is hydrogen and R² is hydrogen.

In certain other specific embodiments, R¹ is hydrogen and R² is halo (e.g., F or Cl) or C₁₋₃ alkyl (e.g., methyl or ethyl).

In other more specific embodiments, R¹ is hydrogen, L₆, L₇ and L₈ are direct bonds, and the compound of Formula (I) has the following structure:

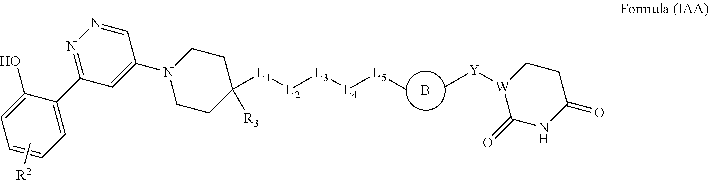

wherein,
R² is hydrogen, halo or C₁₋₃ alkyl;
R³ is halo, C₃₋₆ cycloalkyl, C₆₋₁₂ aryl, —O—R^{e} (wherein R^{e} is C₃₋₆ cycloalkyl or C₁₋₆ alkyl optionally substituted with one or more C₁₋₃alkoxy or halo), C₁₋₆ alkyl optionally substituted with C₁₋₃alkoxy, C₆₋₁₂ aryl, halo, or 5-12 membered heteroaryl optionally substituted with 1 or 2 C₁₋₆ alkyl or C₁₋₆ haloalkyl;
L₁ is —C(O)— or —C(O)—N(R^{c})—, wherein R^{c} is hydrogen, C₁₋₆ alkyl, C₁₋₆ alkoxy, C₁₋₆ haloalkyl, C₃₋₆ cycloalkyl (optionally substituted with 1 or 2 halo, C₁₋₃ alkyl or C₁₋₃ alkoxy), 4-12 membered heterocyclyl (optionally substituted with 1 or 2 C₁₋₃ alkyl), or 5-12 membered heteroaryl (optionally substituted with 1 or 2 C₁₋₃ alkyl or C₁₋₃ haloalkyl), wherein C₁₋₆ alkyl is optionally substituted with 1 or 2 substituents selected from the group consisting of C₁₋₃ alkoxy, CN, 5-12 member heteroaryl (optionally further substituted with 1 or 2 halo or C₁₋₃ alkyl), hydroxy, C₃₋₆ cycloalkyl (optionally further substituted with 1 or 2 halo, C₁₋₃ alkoxy or C₁₋₃ alkyl), phenyl and amino;
L₂ is

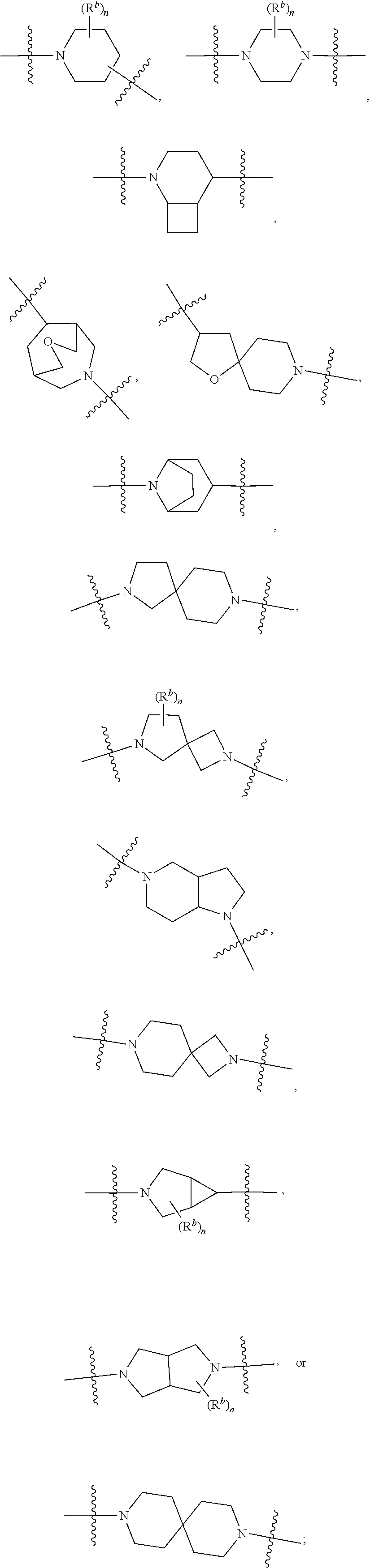

L₃ is direct bond, —CH₂—C(O)—, —(CH₂)₂—O—, or C₁₋₄ alkylene chain optionally substituted with C₁₋₃ alkyl or C₂₋₄ alkylene chain optionally substituted with C₁₋₃ alkyl;
L₄ is

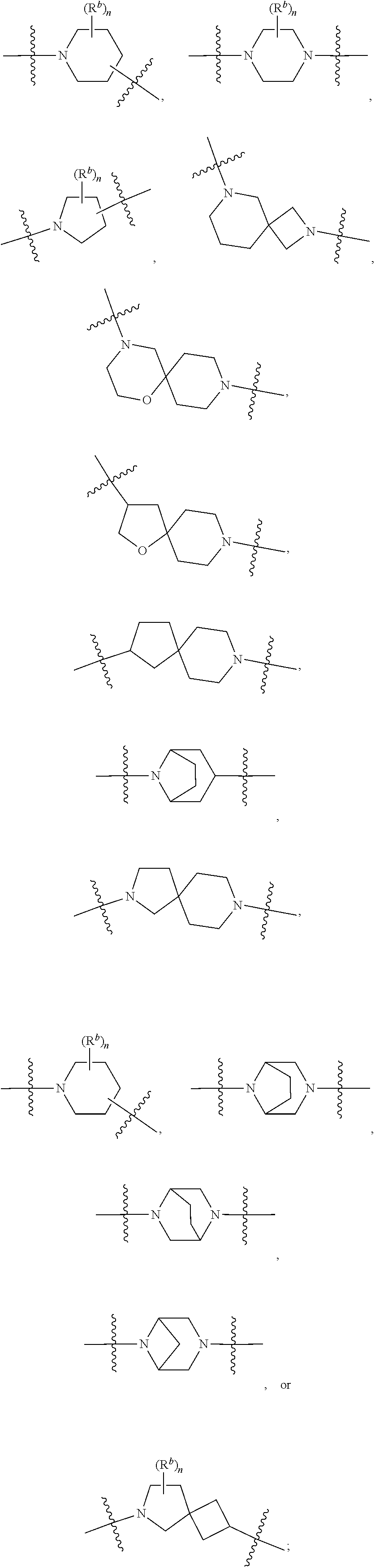

n is 0, 1 or 2, R^{b} is halo, C₁₋₃ alkyl, or C₁₋₃alkoxy;
L₅ is direct bond or —O—,
B is

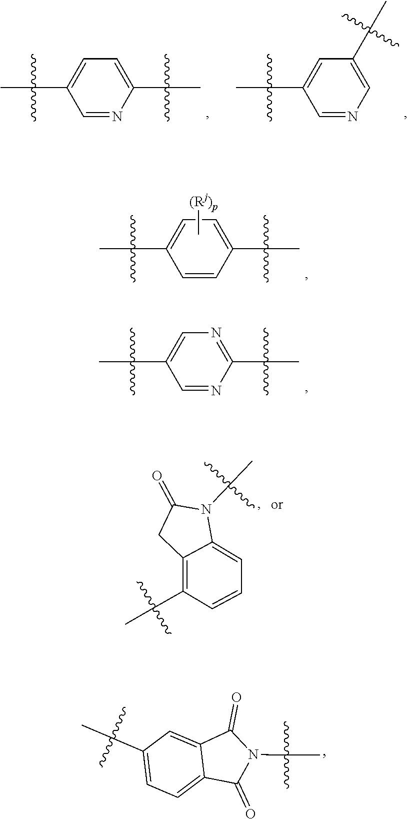

wherein p is 0, 1, or 2, R^{j} is halo;
Y is direct bond, or —C(O)—N(Me)-; and
W is CH or N.

More specifically, in compounds of Formula (IAA), R³ is —O—R^{e} (wherein R^{e} is C₃₋₆ cycloalkyl or C₁₋₆ alkyl), C₁₋₆ alkyl optionally substituted with C₁₋₃ alkoxy, 5-12 membered heteroaryl optionally substituted with 1 or 2 C₁₋₆ alkyl or C₁₋₆ haloalkyl;
L₁ is —C(O)— or —C(O)—N(R^{c})—, wherein R^{c} is hydrogen, C₁₋₆ alkyl, C₁₋₆ alkoxy, C₃₋₆ cycloalkyl, or C₁₋₆ haloalkyl, wherein C₁₋₆ alkyl is optionally substituted with 1 or 2 C₁₋₃ alkoxy, hydroxy, C₃₋₆ cycloalkyl, phenyl or amino;
L₂ is

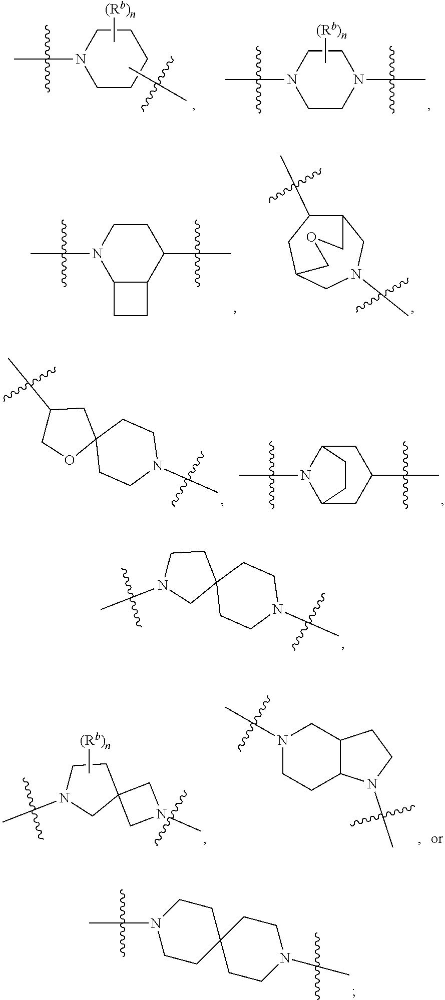

L₃ is direct bond, —CH₂—C(O)— or C₁₋₂ alkylene chain optionally substituted with C₁₋₃ alkyl;
L₄ is

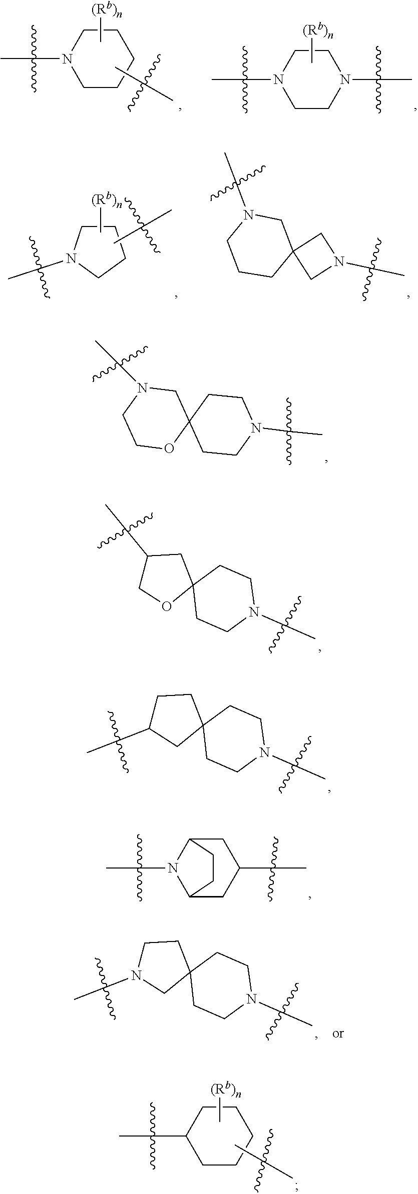

n is 0, 1 or 2, R^{b} is halo or C₁₋₃alkyl;
L₅ is direct bond or —O—,
B is

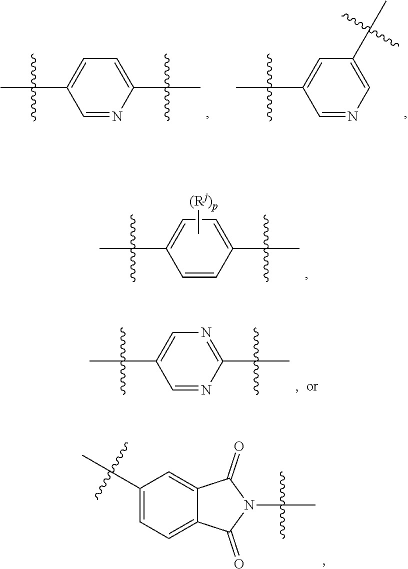

wherein p is 0, 1, or 2, R¹ is halo;
Y is direct bond, or —C(O)—N(Me)-; and
W is CH or N.

In further more specific embodiments, Y is direct bond, W is CH, and the compound of Formula (IAA) are represented by the following structure:

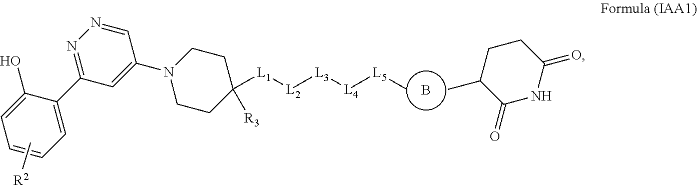

In further more specific embodiments, L₁ is —C(O)— or —C(O)—N(R^{c})—(R^{c} is hydrogen, C₁₋₆ alkyl, C₁₋₆ alkoxy, C₃₋₆ cycloalkyl, or C₁₋₆ haloalkyl, wherein C₁₋₆ alkyl is optionally substituted with 1 or 2 C₁₋₃ alkoxy, hydroxy, C₃₋₆ cycloalkyl, phenyl or amino), and
-L₂-L₃-L₄- has the following structures:

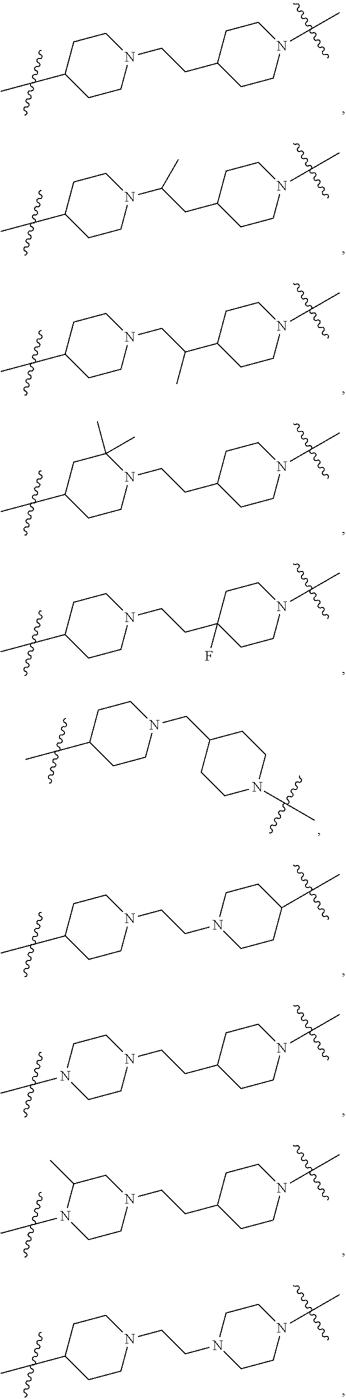

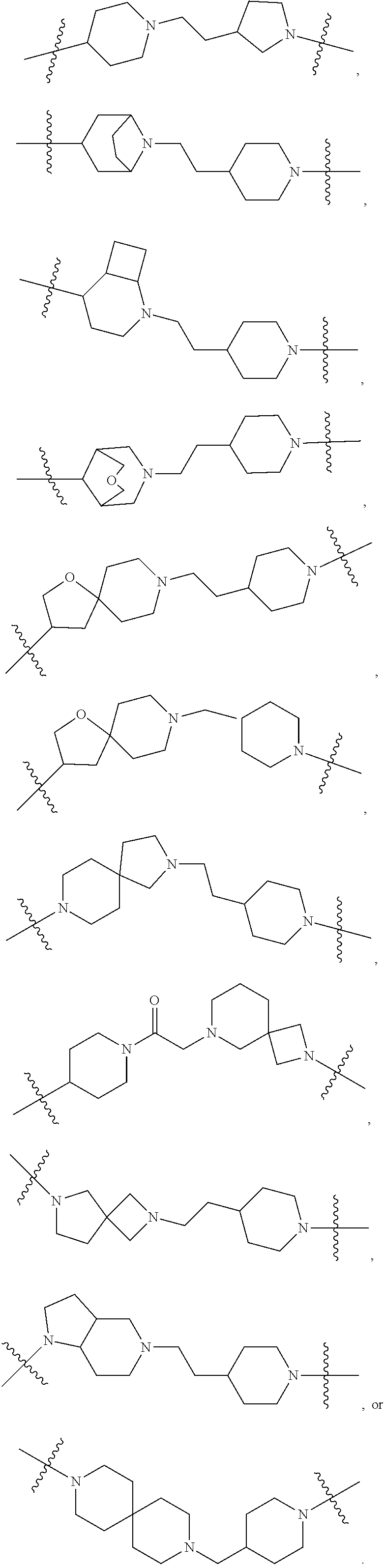

In further more specific embodiments, R³ is —O—R^{e} (wherein R^{e} is methyl, ethyl, propyl, cyclopropyl or cyclobutyl), —CH₂OCH₃, thiazol-4yl, thiazol-5yl, 1-methyl-1H-pyrazol-5yl, 1-ethyl-1H-pyrazol-5yl, 1,3-dimethyl-1H-pyrazol-5-yl, 1,4-dimethyl-1H-pyrazol-5-yl;
L₁ is C(O), or —C(O)—N(R^{c})—, wherein R^{c} is hydrogen, methyl, ethyl, propyl, isopropyl, cyclopropyl, cyclobutyl, —OCH₃, —CH₂CF₃, —CH₂CH₂—N(CH₃)₂, —CH₂CH₂—OCH₃, —CH₂-cyclopropyl, benzyl, or 1,3-dihydroxypropan-2yl; and
B is

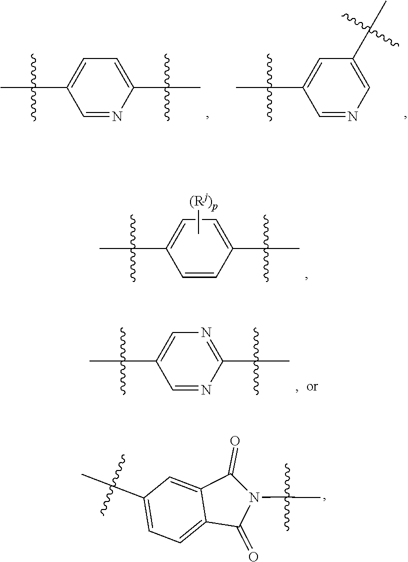

wherein p is 0, 1, or 2, R^{j} is F.

In other more specific embodiments, L₁ is —C(O)— or —C(O)—N(R^{c})—(R^{c} is hydrogen, C₁₋₆ alkyl, C₁₋₆ alkoxy, C₃₋₆ cycloalkyl, or C₁₋₆ haloalkyl, wherein C₁₋₆ alkyl is optionally substituted with 1 or 2 C₁₋₃ alkoxy, hydroxy, C₃₋₆ cycloalkyl, phenyl or amino),
L₃ is a direct bond, and
-L₂-L₃-L₄- has the following structures:

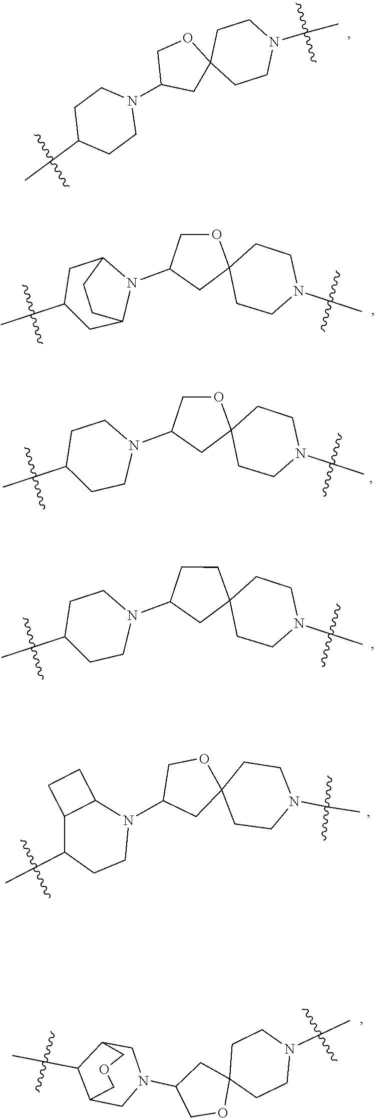 [SMILES: *C1CCN(C2COC3(CCN(*)CC3)C2)CC1 ; *C1CC2CCC(C1)N2C1COC2(CCN(*)CC2)C1 ; *C1CCN(C2COC3(CCN(*)CC3)C2)CC1 ; *C1CCN(C2CCC3(CCN(*)CC3)C2)CC1 ; *C1CCN(C2COC3(CCN(*)CC3)C2)C2CCC12 ; *C1C2COCC1CN(C1COC3(CCN(*)CC3)C1)C2]

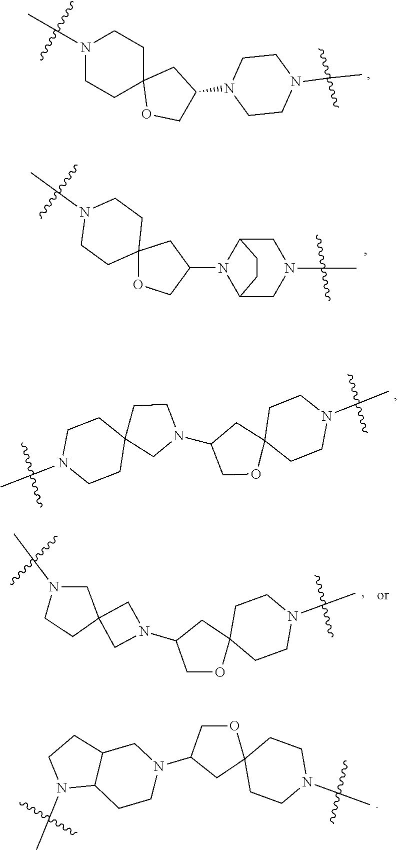 [SMILES: *N1CCN([C@@H]2COC3(CCN(*)CC3)C2)CC1 ; *N1CCC2(CC1)CC(N1C3CCC1CN(*)C3)CO2 ; *N1CCC2(CC1)CCN(C1COC3(CCN(*)CC3)C1)C2 ; *N1CCC2(CC1)CC(N1CC3(CCN(*)C3)C1)CO2 ; *N1CCC2(CC1)CC(N1CCC3C(CCN3*)C1)CO2]

In yet more specific embodiments, R³ is —O—R^{e} (wherein R^{e} is methyl, ethyl, propyl, cyclopropyl or cyclobutyl), thiazol-4yl, thiazol-5yl, 1-methyl-1H-pyrazol-5yl, 1-ethyl-1H-pyrazol-5yl, 1,3-dimethyl-1H-pyrazol-5-yl, 1,4-dimethyl-1H-pyrazol-5-yl, 1-(oxan-4-yl)-1H-pyrazol-5-yl, 1-methyl-3-(trifluoromethyl)-1H-pyrazol-5-yl, or 2-methyl-2H-indazol-3-yl;
L₁ is C(O), or —C(O)—N(R^{c})—, wherein R^{c} is hydrogen, methyl, ethyl, propyl, isopropyl, cyclopropyl, cyclobutyl, —CH₂CF₃, —CH₂CH₂—N(CH₃)₂, —CH₂CH₂—OCH₃, or benzyl; and
B is

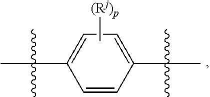

wherein p is 0, 1, or 2, R^{j} is halo.

In further more specific embodiments, R³ is pyrazol-5yl (optionally substituted with 1 or 2 methyl, trifluoromethyl, oxan-4-yl), thiazolyl, or indazolyl (optionally substituted with 1 or 2 methyl), —CH₂OCH₃, or O-cyclobutyl.

According to other more specific embodiments, in compounds of Formula (IAA1): R² is hydrogen, F, Cl or methyl; R³ is F, cyclopropyl, phenyl, benzyl, C₁₋₃ haloalkyl, C₁₋₆ alkyl (optionally substituted 1 or 2 C₁₋₃ alkoxy), or —O—R^{e} (wherein R^{e} is cyclopropyl, cyclobutyl, or C₁₋₆ alkyl optionally substituted with one or more C₁₋₃alkoxy or halo); L₁ is —C(O)—N(R^{c})—, wherein R^{c} is C₁₋₆ alkyl, C₁₋₆ alkoxy, cyclopropyl (optionally substituted with methyl or methoxy), cyclobutyl (optionally substituted with 1 or 2 methyl or F), azetidinyl (optionally substituted with 1 or 2 methyl), pyrazolyl (optionally substituted with difluoromethyl), phenyl, or C₁₋₆ haloalkyl, wherein C₁₋₆ alkyl is optionally substituted with 1 or 2 C₁₋₃ alkoxy, CN, oxazolyl, thiazolyl (optionally substituted with methyl), pyrazinyl, pyrimidinyl, 1-methyl-1,2,3-triazolyl, pyrazolyl (optionally substituted with 1 or 2 C₁₋₃ alkyl), oxadiazolyl (optionally substituted with methyl), cyclopropyl (optionally substituted with C₁₋₃ alkoxy), cyclobutyl, or 3,3-difluorocyclobutyl);
-L₂-L₃-L₄-L₅- is

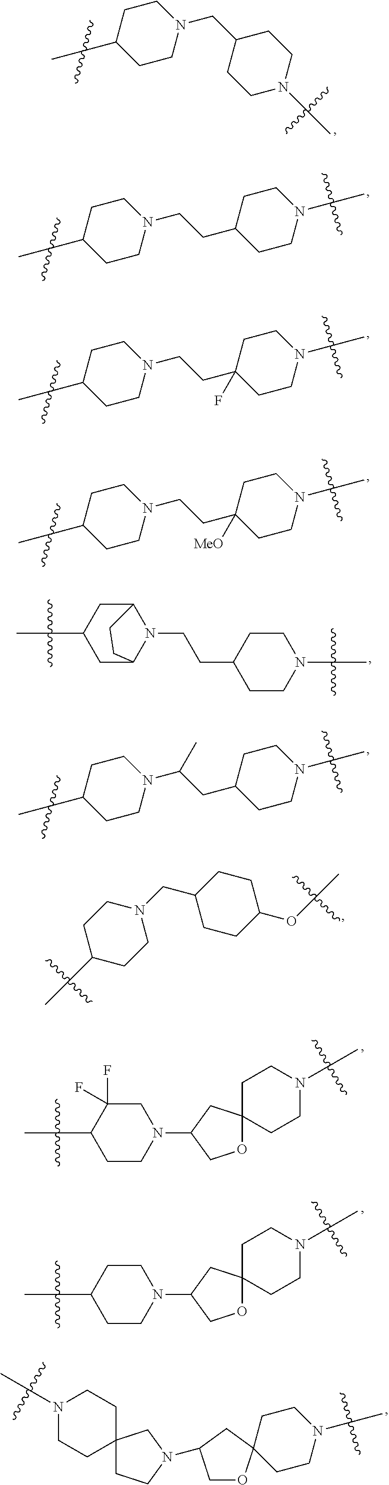

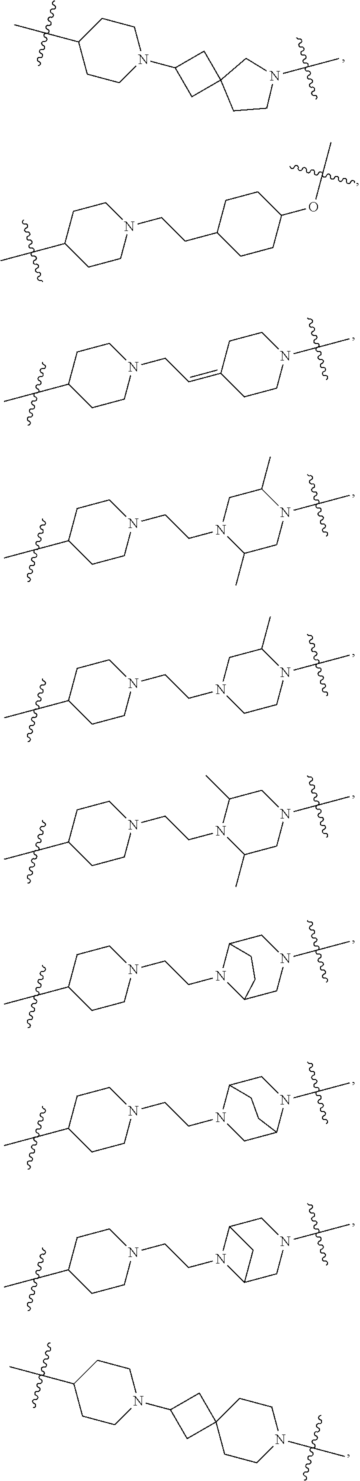

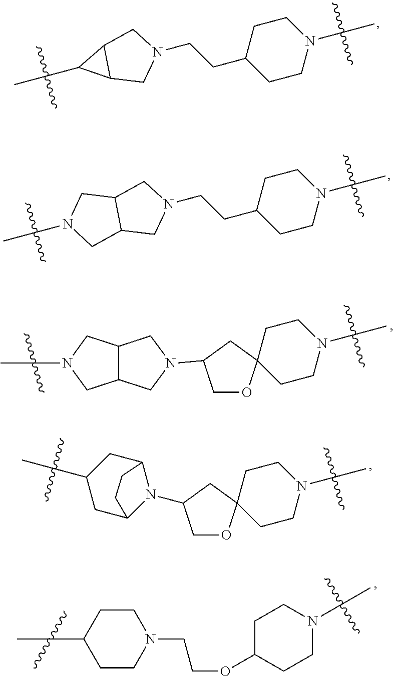 [SMILES: *C1C2CN(CCC3CCN(*)CC3)CC12 ; *N1CCC(CCN2CC3CN(*)CC3C2)CC1 ; *N1CCC2(CC1)CC(N1CC3CN(*)CC3C1)CO2 ; *C1CC2CCC(C1)N2C1COC2(CCN(*)CC2)C1 ; *C1CCN(CCOC2CCN(*)CC2)CC1]

According to other more specific embodiment, in compounds of Formula (IAA1):
R² is hydrogen or F; R³ is C₁₋₆ alkyl (optionally substituted 1 or 2 C₁₋₃ alkoxy), or —O—R^{e} (wherein R^{e} is C₁₋₆ alkyl optionally substituted with one or more C₁₋₃ alkoxy or halo); L₁ is —C(O)—; and
-L₂-L₃-L₄-L₅- is

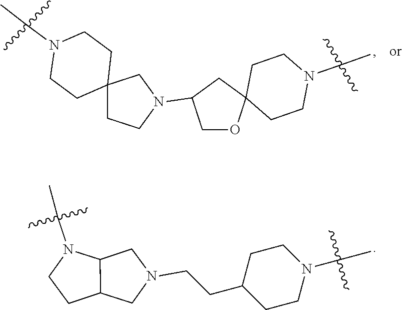 [SMILES: *N1CCC2(CC1)CCN(C1COC3(CCN(*)CC3)C1)C2 ; *N1CCC(CCN2CC3CCN(*)C3C2)CC1]

In other various embodiments, the compounds of Formula (IAA) may be represented by the structures of Formula (IAA2) or Formula (IAA3):

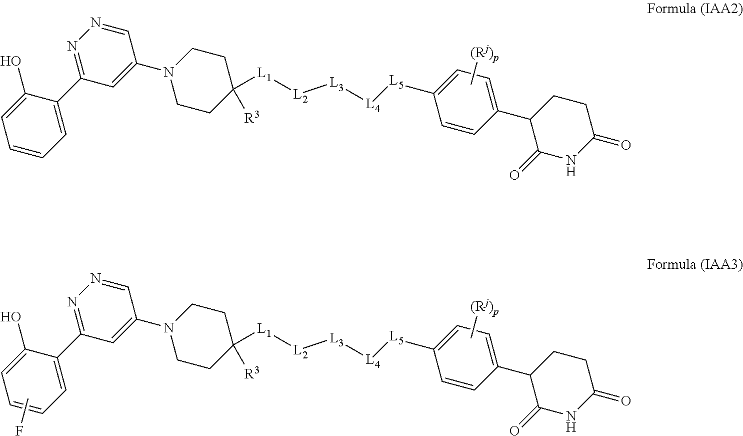

wherein,
R³ is —O—R^{e} (wherein R^{e} is C₃₋₆ cycloalkyl or C₁₋₆ alkyl), C₁₋₆ alkyl optionally substituted with C₁₋₃ alkoxy, or 5-12 membered heteroaryl optionally substituted with 1 or 2 C₁₋₆ alkyl or C₁₋₆ haloalkyl;
L₁ is C(O), or —C(O)—N(R^{c})—, wherein R^{c} is hydrogen, C₁₋₆ alkyl, C₁₋₆ alkoxy, C₃₋₆ cycloalkyl, or C₁₋₆ haloalkyl, wherein C₁₋₆ alkyl is optionally substituted with 1 or 2 C₁₋₃ alkoxy, hydroxy, C₃₋₆ cycloalkyl, phenyl or amino;
-L₂-L₃-L₄-L₅- is

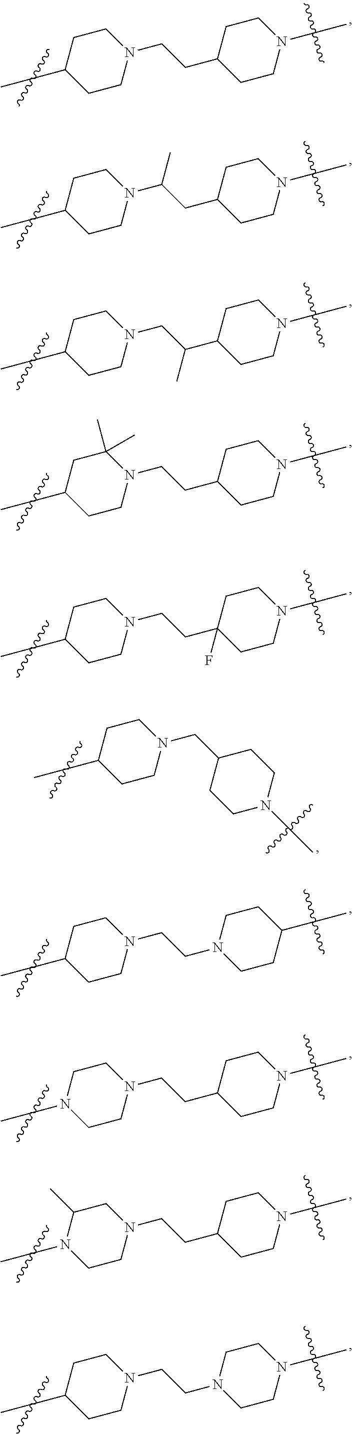

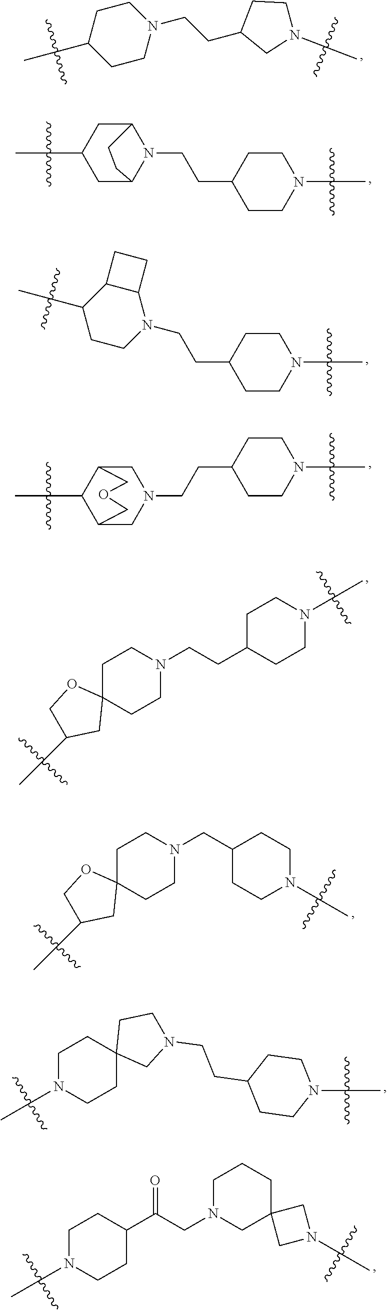

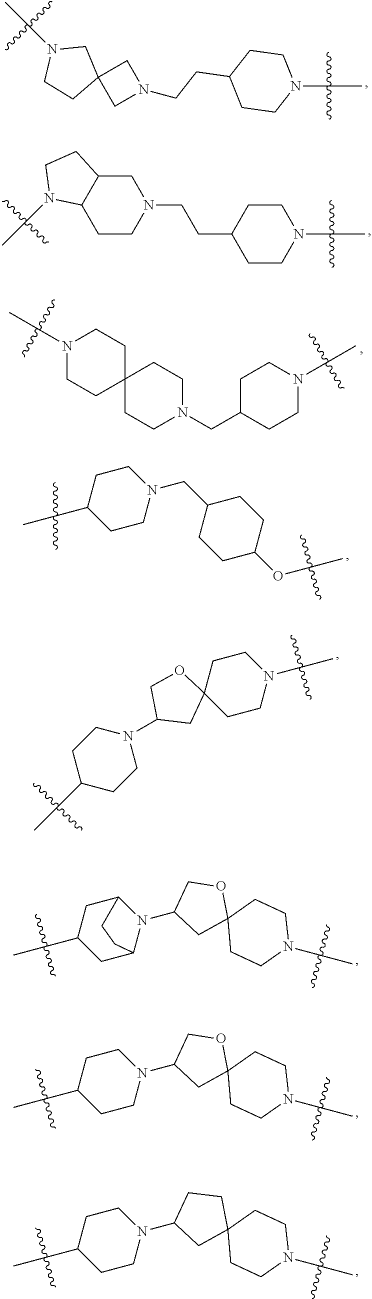

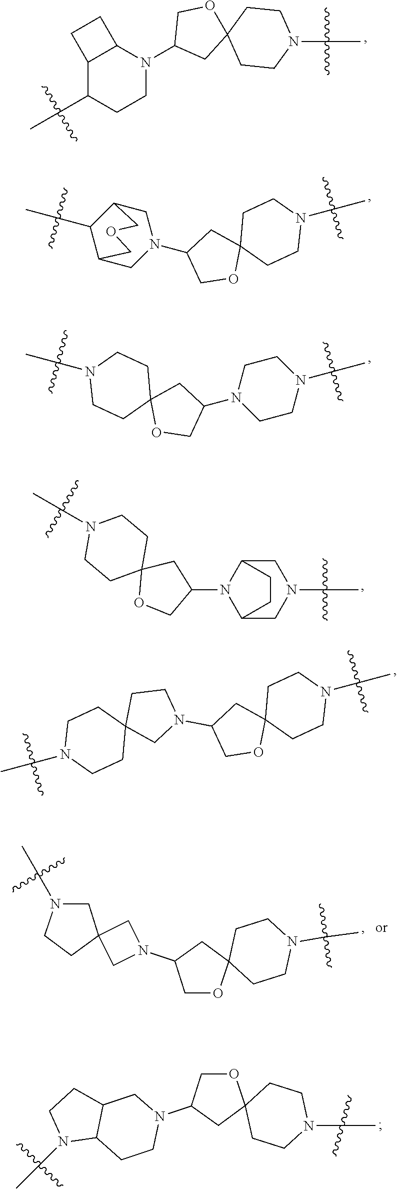

p is 0, 1, or 2; and
R^{j} is halo.

In various more specific embodiments, R^{c} is C₁₋₆ alkoxy, C₃₋₆ cycloalkyl, C₁₋₆ haloalkyl, or C₁₋₆ alkyl substituted with 1 or 2 C₁₋₃ alkoxy, hydroxy, C₃₋₆ cycloalkyl, phenyl or amino.

In various specific embodiments, R³ is OR^{e}, and R^{e} is methyl, ethyl, or cyclopropyl.

In various more specific embodiments, R³ is pyrazol-5yl (optionally substituted with 1 or 2 methyl, trifluoromethyl, oxan-4-yl), thiazolyl, or indazolyl (optionally substituted with 1 or 2 methyl), —CH₂OCH₃, or O-cyclobutyl.

In other various embodiments, R¹ is hydrogen, R² is hydrogen, L₆, L₇ and L₈ are direct bonds, and W is N, the compound of Formula (IAA) may be represented by the structure of Formula (IAA4):

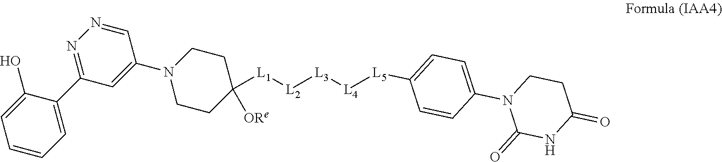

wherein,
R^{e} is C₁₋₆ alkyl;
L₁ is —C(O)—N(R^{c})—, wherein R^{c} is hydrogen, or C₁₋₆ alkyl;
-L₂-L₃-L₄-L₅- is

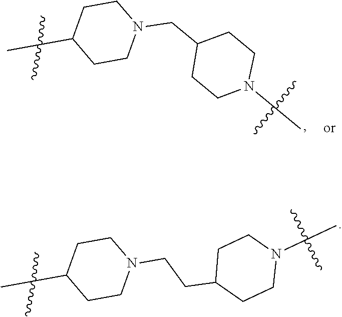 [SMILES: *C1CCN(CC2CCN(*)CC2)CC1 ; *C1CCN(CCC2CCN(*)CC2)CC1]

In other various embodiments, R¹ is hydrogen, R² is hydrogen, Y is —C(O)—N(CH₃)—, L₆, L₇ and L₈ are direct bonds, the compound of Formula (IAA) may be represented by the structure of Formula (IAA5):

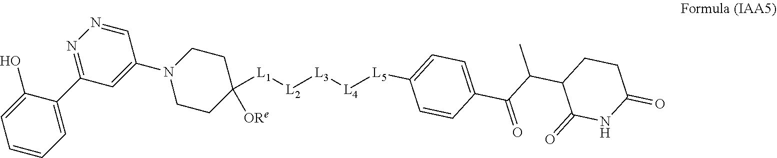

wherein,
R^{e} is C₁₋₆ alkyl;
L₁ is —C(O)—N(R^{c})—, wherein R^{c} is hydrogen, or C₁₋₆ alkyl;
-L₂-L₃-L₄-L₅- is

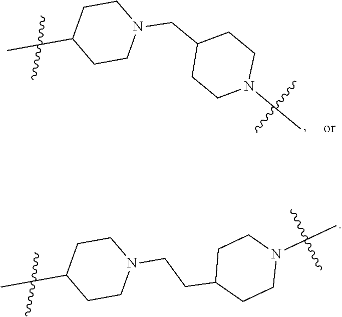 [SMILES: *C1CCN(CC2CCN(*)CC2)CC1 ; *C1CCN(CCC2CCN(*)CC2)CC1]

In yet other embodiments, the compound of Formula (IB) is represented by the following structure:

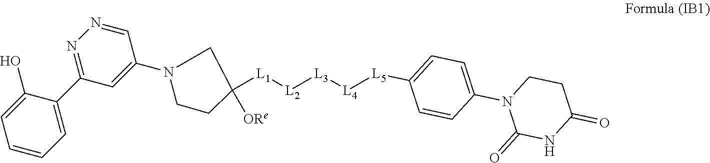

wherein,
R^{e} is C₁₋₆ alkyl;
L₁ is —C(O)—N(R^{c})—, wherein R^{c} is hydrogen, or C₁₋₆ alkyl;
-L₂-L₃-L₄-L₅- is

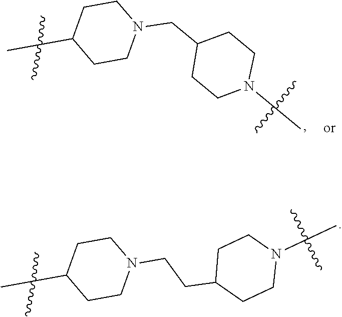 [SMILES: *C1CCN(CC2CCN(*)CC2)CC1 ; *C1CCN(CCC2CCN(*)CC2)CC1]

In more specific embodiments, compounds of Formula (II) are represented Formula (IB2):

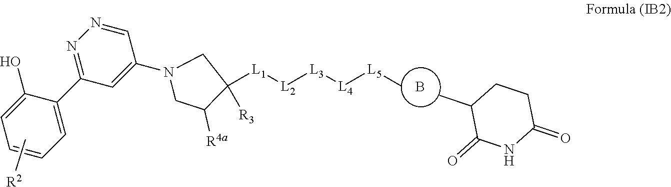

wherein,
R² is hydrogen or halo;
R³ is hydrogen, halo, C₆₋₁₂ aryl, C₁₋₆ alkyl optionally substituted with C₁₋₃alkoxy, or —O—R^{e} (wherein R^{e} is C₃₋₆ cycloalkyl, or C₁₋₆ alkyl optionally substituted with one or more C₁₋₃alkoxy or halo);
R^{4a} is hydrogen, C₁₋₆ alkyl or C₁₋₆ alkoxy;
L₁ is —C(O)—, —C(O)—N(R^{c})— or —CH₂—C(O)—N(R^{c})—, wherein R^{c} is hydrogen, C₁₋₆ haloalkyl, C₃₋₆ cycloalkyl (optionally substituted with 1 or 2 halo, C₁₋₃ alkyl or C₁₋₃ alkoxy), or C₁₋₆ alkyl (optionally substituted with 1 or 2 C₁₋₃ alkoxy or 5-12 membered heteroaryl (optionally further substituted with 1 or 2 halo or C₁₋₃ alkyl));
L₂ is

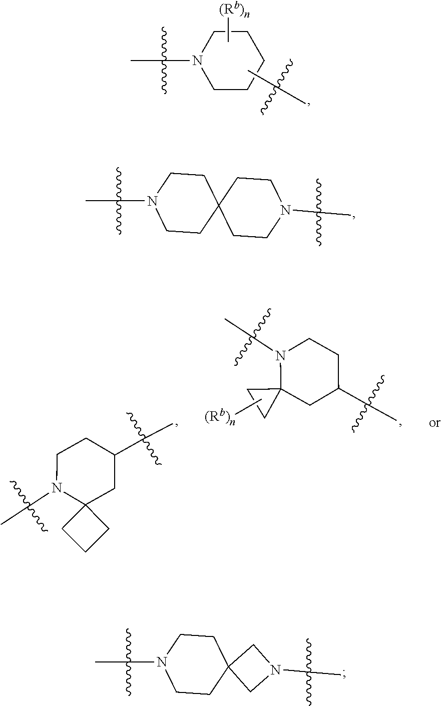

L₃ is direct bond, C₁₋₄ alkylene chain optionally substituted with C₁₋₃ alkyl or C₂₋₄ alkylene chain optionally substituted with C₁₋₃ alkyl;
L₄ is

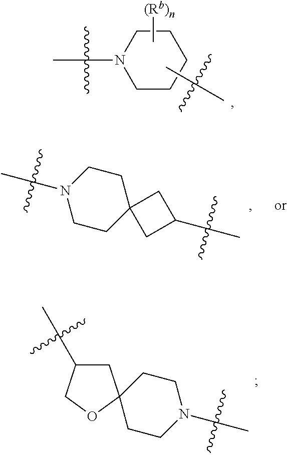

n is 0, 1 or 2, R^{b} is halo, C₁₋₃ alkyl, or hydroxy;
L₅ is direct bond or —O—,
B is

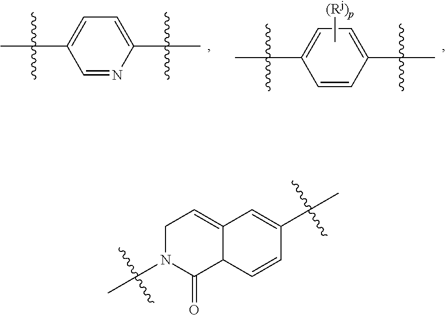

wherein p is 0, 1, or 2, R^{j} is halo.

More specifically, R² is hydrogen or F; R³ is F, C₁₋₃ alkyl, phenyl, or —O—R^{e} (wherein R^{e} is cyclopropyl or C₁₋₃ alkyl); R^{4a} is hydrogen, or methoxy; L₁ is —C(O)— or —C(O)—N(R^{c})—, wherein R^{c} is hydrogen, C₁₋₆ alkyl, C₁₋₃ alkyl (optionally substituted with methoxy, oxazolyl, thiazolyl, pyrazinyl, pyrimidinyl, 1-methyl-1,2,3-triazolyl, pyrazolyl (optionally substituted with 1 or 2 C₁₋₃ alkyl), oxadiazolyl (optionally substituted with methyl), or cyclopropyl (optionally substituted with 1 or 2 F, methyl, or methoxy);
-L₂-L₃-L₄-L₅- is

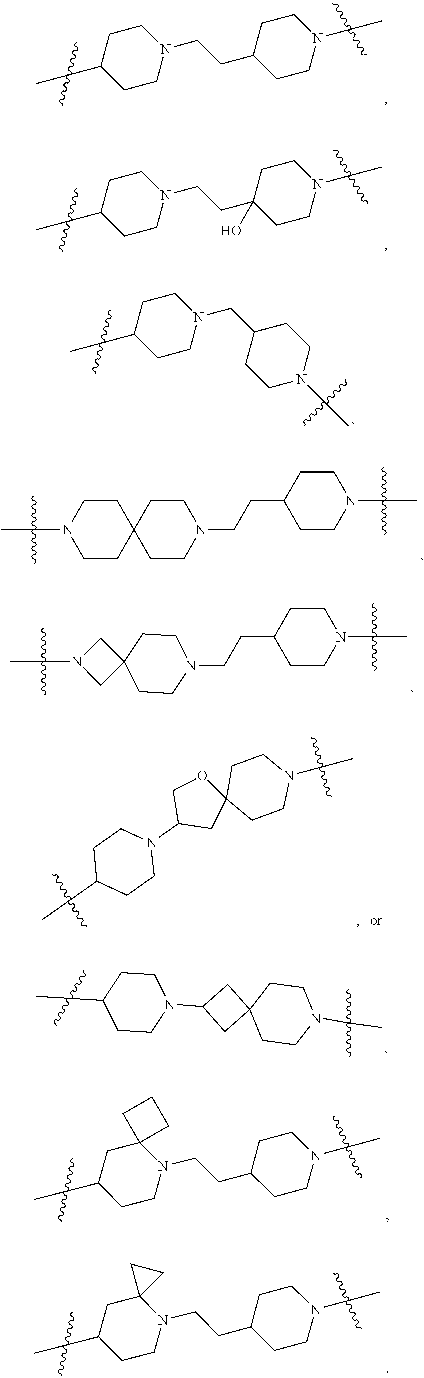

In more specific embodiments, compounds of Formula (II) are represented Formula (IC1) or (IC2):

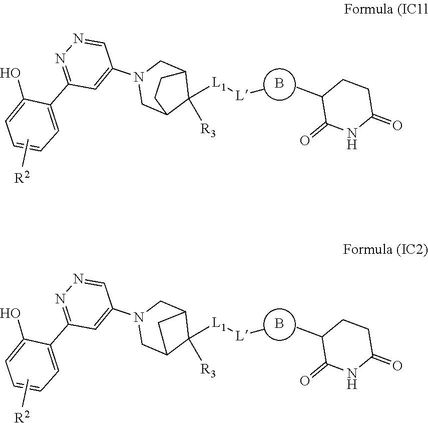

wherein, R² is hydrogen or halo; R³ is hydrogen or C₁₋₃ alkyl; L₁ is —C(O)—N(R^{c})—, wherein R^{c} is hydrogen, C₁₋₆ haloalkyl, C₁₋₆ alkoxy, C₃₋₆ cycloalkyl or C₁₋₆ alkyl optionally substituted with C₃₋₆ cycloalkyl, 5-7 member heteroaryl (preferably thiazolyl), or C₁₋₆ alkoxy;
L′ is

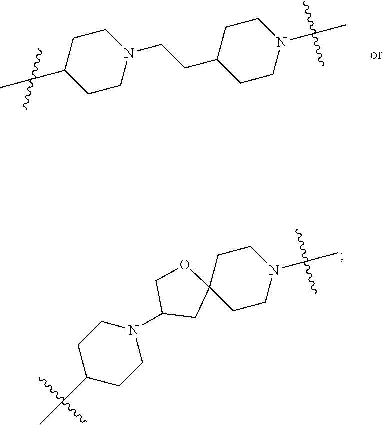 [SMILES: *C1CCN(CCC2CCN(*)CC2)CC1 ; *C1CCN(C2COC3(CCN(*)CC3)C2)CC1]

and
B is

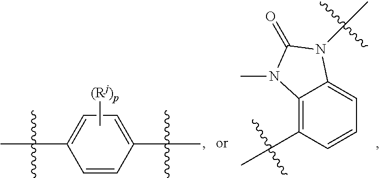

wherein p is 0, 1, or 2, R^{j} is halo.

In more specific embodiments, compounds of Formula (II) are represented Formula (ID1):

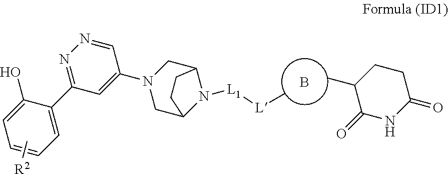

wherein, R² is hydrogen or halo; R³ is hydrogen or C₁₋₃ alkyl; L₁ is —C(O)—;
L′ is

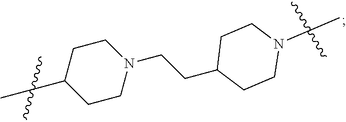 [SMILES: *C1CCN(CCC2CCN(*)CC2)CC1]

and B is

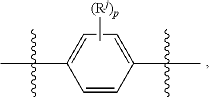

wherein p is 0, 1, or 2, R^{j} is halo.

In more specific embodiments, compounds of Formula (II) are represented Formula (IE1):

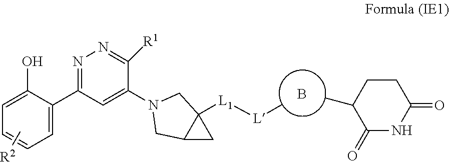

wherein, R² is hydrogen or halo; L₁ is —C(O)—N(R^{c})—, wherein R^{c} is hydrogen or C₁₋₆ alkyl;
L′ is

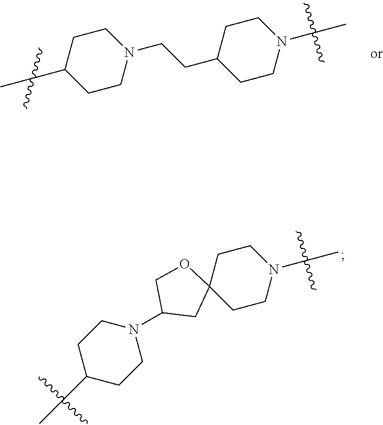 [SMILES: *C1CCN(CCC2CCN(*)CC2)CC1 ; *C1CCN(C2COC3(CCN(*)CC3)C2)CC1]

and B is

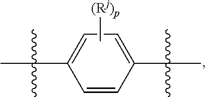

wherein p is 0, 1, or 2, R^{j} is halo.

In more specific embodiments, compounds of Formula (II) are represented Formula (IF1)

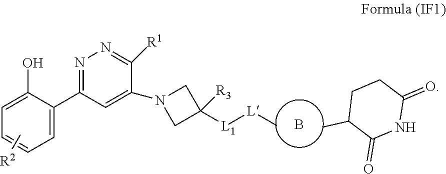

wherein, R² is hydrogen or halo; R³ is hydrogen, C₁₋₆ alkyl, or —OR^{e} (wherein R³ is C₁₋₆ alkyl); L₁ is —C(O)—N(R^{c})—, wherein R^{c} is hydrogen or C₁₋₆ alkyl;
L′ is

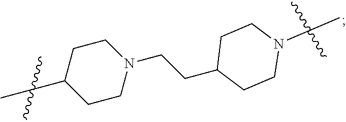 [SMILES: *C1CCN(CCC2CCN(*)CC2)CC1]

and B is

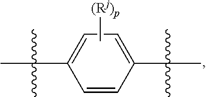

wherein p is 0, 1, or 2, R^{j} is halo.

### SMARCA2 Binders

Each bifunctional compound of Formulae (I) and (IA) comprises a selective SMARCA1 binder moiety, depicted as Formula (A):

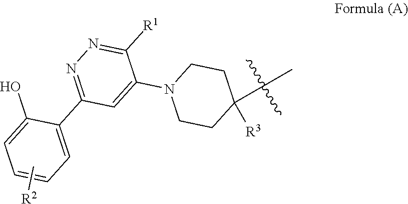

wherein,

In more specific embodiments, R¹ and R² are each hydrogen.

In other more specific embodiments, R¹ is hydrogen, R² is halo (e.g., F or Cl) or C₁₋₃ alkyl (e.g., methyl or ethyl).

In specific embodiments, R¹ is hydrogen, R² is hydrogen, F, Cl or methyl, R³ is OR^{e} (R^{e} is defined as herein), and the SMARCA2 binder moiety has one of the following structures:

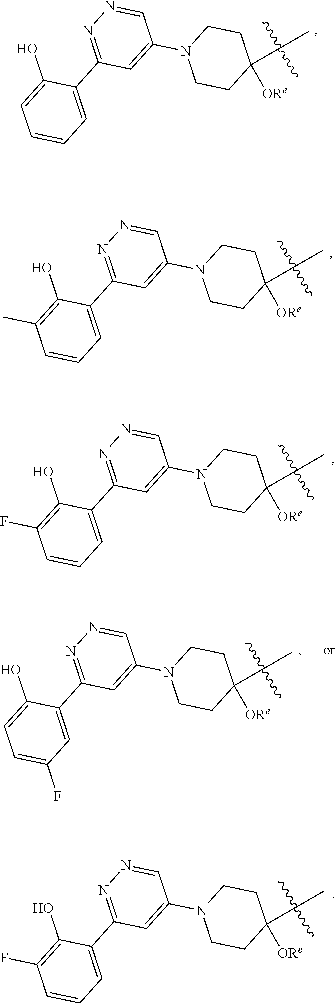

In other specific embodiments, R¹ is hydrogen, R² is hydrogen, R³ is heteroaryl (e.g., pyrazol-5yl (optionally substituted with 1 or 2 methyl, trifluoromethyl, oxan-4-yl), thiazolyl, or indazolyl (optionally substituted with 1 or 2 methyl)), or alkyl optionally substituted with alkoxy.

### Ligase Harness Moieties (LHM)

The LHM moiety of the bifunctional compound of Formula (I) targets CRBN of E3 ligases. Once harnessed by the bifunctional compounds, the E3 ligases are capable of inducing ubiquitination and subsequent proteasomal degradation of SMARCA2.

The LHM moiety of Formula (I) typically comprises a glutarimide or dihydrouracil moiety coupled to a ring structure, as represented by Formula (B):

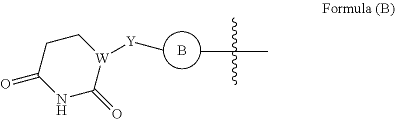

wherein,
W is —C(R^{g})— or —N—;
Y is direct bond, C₁₋₄ alkylene chain, —C(O)—, —C(O)O—, —O—, —N(R^{g})—, —S— —C(S)—, —C(S)—O—, —O—C(O)O—, —C(O)—N(R^{g})—, or —O—C(O)—N(R^{g})—;
B ring is C₆₋₁₂ aryl, 5-12 membered heteroaryl, or 4-12 membered heterocyclyl, each being optionally substituted with 1 to 3 R^{j};
R^{g} is hydrogen or C₁₋₆ alkyl; and
each R^{j} is independently halo, oxo, —CN, —O—R^{c}, C₁₋₆ alkyl, or C₁₋₆ haloalkyl.

In more specific embodiments, W is —CH— and Y is direct bond.

In more specific embodiments, B ring is:

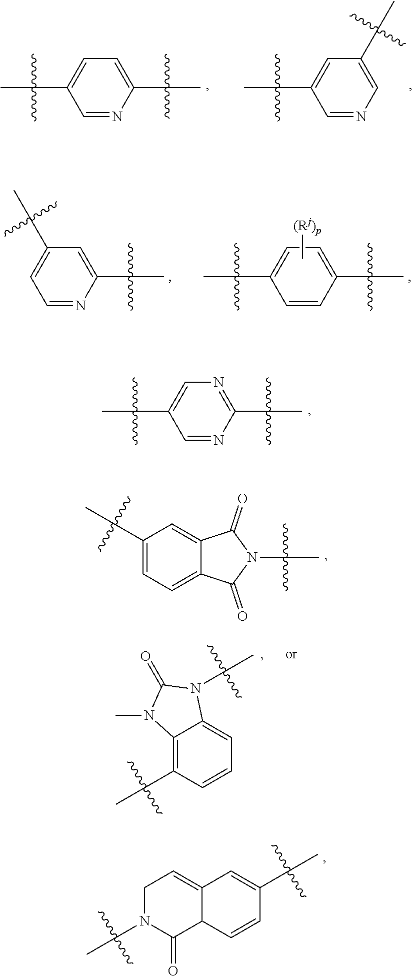

wherein p is 0, 1 or 2, R^{j} is halo, —CN, —O—R^{c}, C₁₋₆ alkyl, or C₁₋₆ haloalkyl.

In yet other embodiments, W is —CH—, Y is —N(CH₃)—C(O)—, and B ring is

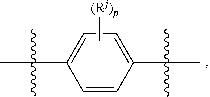

wherein p is 0, 1 or 2, R^{j} is halo.

In other embodiments, W is —N—, Y is direct bond; and B ring is

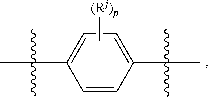

wherein p is 0, 1 or 2, R^{j} is halo.

In more specific embodiments, the LHM moiety is:

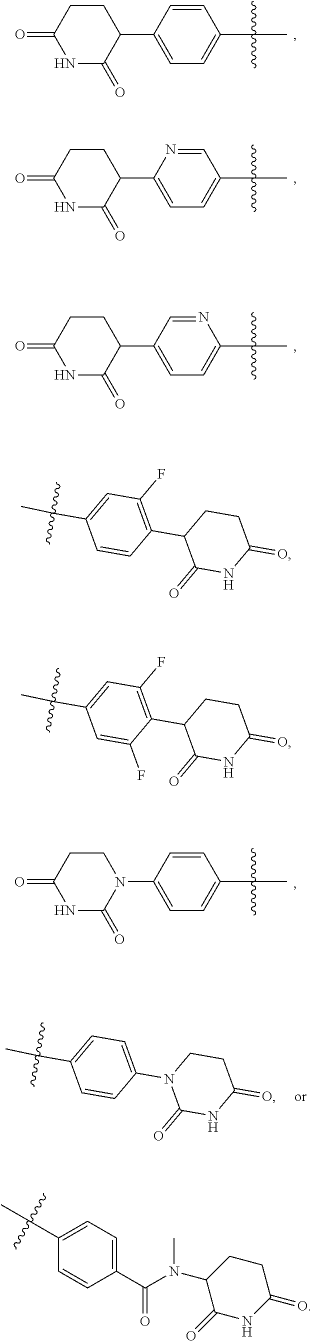

### Linker

Each bifunctional compound of Formula (I), Formula (II), or substructures represented by any one of Formulae (IA), (IB), (IC), (ID), (IE), (IF), (IAA), (IAA1), (IAA2), (IAA3), (IAA4), (IAA5), (IB1), (IB2), (IC1), (IC2), (ID1), (IE1), or (IF1) comprises a Linker (L), which is a bivalent moiety that couples the SMARCA2 Binder moiety to the LHM. The structure (e.g., length or rigidity) of the linker moiety may impact the efficiency or selectivity of the degradation process. The Linker comprises a continuous sequence of covalent bonds between the respective attachment points to the SMARCA2 Binder moiety and the LHM, inclusive of the bond indicated by a wavy line of Formula (I) and its substructures.

Typically, the linker moiety comprises multiple bivalent segments (i.e., -L₁-L₂-L₃-L₄-L₅-L₆-L₇-L₈-), which collectively contribute to the overall length and rigidity of the Linker, in addition to providing the respective attachment points to the SMARCA2 Binder moiety and the LHM. In various embodiments, the linker segments L₁, L₂, L₃, L₄, L₅, L₆, L₇, L₈ are each independently:
i) C₃₋₁₂ cycloalkyl optionally substituted with 1-3 R^{b}.
ii) C₆₋₁₂ aryl optionally substituted with 1-3 R^{b};
iii) 4-12 membered heterocyclyl optionally substituted with 1-3 R^{b};
iv) 5-12 membered heteroaryl optionally substituted with 1-3 R^{b};
v) direct bond;
vi) C₁₋₁₂ alkylene chain optionally substituted with 1-3 R^{b};
(vii) C₁₋₁₂ alkenylene chain optionally substituted with 1-3 R^{b}; or
viii) —(CH₂)ₘ—C(O)—, —(CH₂)ₘ—C(O)O—, —(CH₂)ₘ—O—, —(CH₂)ₘ—N(R^{c})—, —(CH₂)ₘ—S—, —(CH₂)ₘ—C(S)—, —(CH₂)ₘ—C(S)—O—, —(CH₂)ₘ—S(O)₂—, —(CH₂)ₘ—S(O)=N—, —(CH₂)ₘ—S(O)₂NH—, —(CH₂)ₘ—C(O)—N(R^{c})—, —C(O)—N(R^{c})—(CH₂)ₘ—, —(CH₂)ₘ—O—C(O)—N(R^{c})—, —(CH₂)ₘ—O—C(O)—O—, or —NH—(CH₂)ₘ—C(O)—, wherein m is 0, 1, 2, 3, 4, 5 or 6;
wherein R^{d}, R^{c} and R^{d} are as defined herein.

It is to be understood that, unless otherwise specified and provided that the valence is satisfied, the bivalent linker segments described herein (e.g., L¹ or L²) are not limited to the direction in which they are expressed. For instance, for a given linker segment, e.g., —C(O)—NH—, the manner in which it is connected to the remainder of the molecule may be either direction: i.e., —C(O)—NH— or —NH—C(O)—, provided that the connection does not violate valence rules.

Unless other specified, it is further to be understood that the first linker segment L¹ is directly coupled to the SMARCA2 Binder moiety, whereas the last linker segment L⁸ is directly coupled to the LHM.

One or more linker segments may be direct bonds. To illustrate, in a sequence of linker segments represented by -L₂-L₃-L₄-, when L₃ is a direct bond, it is effectively absent because L₂ and L₄ are coupled directly to each other.

In another embodiment, the Linker has one or more rings, which tend to increase the linker rigidity. A combination of chain bonds and ring(s) may be used to tune the relative orientations of the bifunctional groups or the distance therebetween.

In various specific embodiments, the linker (L) of compounds of Formulae (I), (II), (IA), (IB), (IC), (ID), (IE), (IF), (IAA), (IAA1), (IAA2), (IAA3), (IAA4), (IAA5), (IB1), (IB2), (IC1), (IC2), (ID1), (IE1), or (IF1) is presented by -L₁-L₂-L₃-L₄-L₅-L₆-L₇-L₈-, each L₁, L₂, L₃, L₄, L₅, L₆, L₇, or L₈, being independently:
i) a bivalent ring moiety selected from the group consisting of:

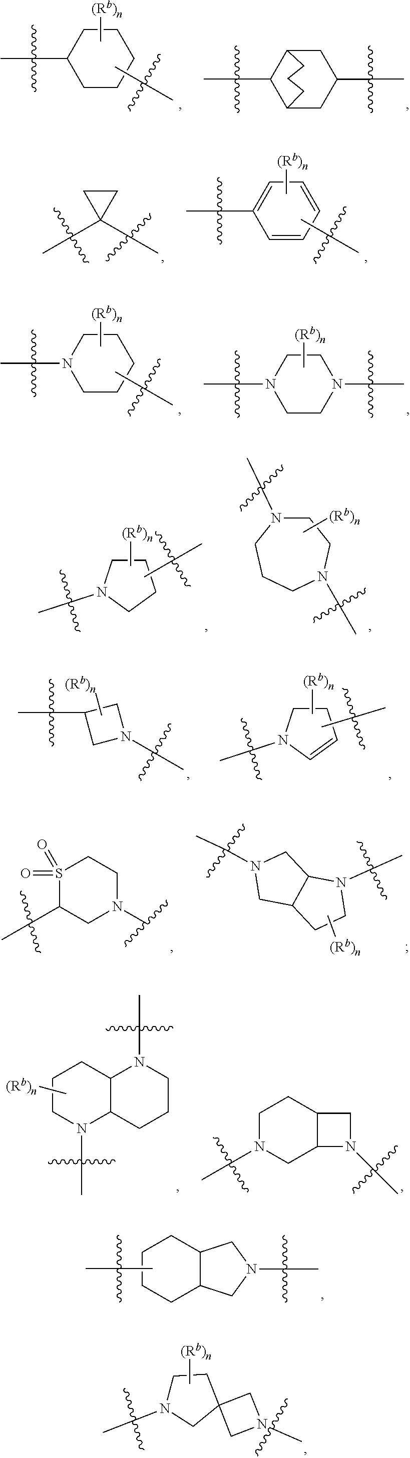

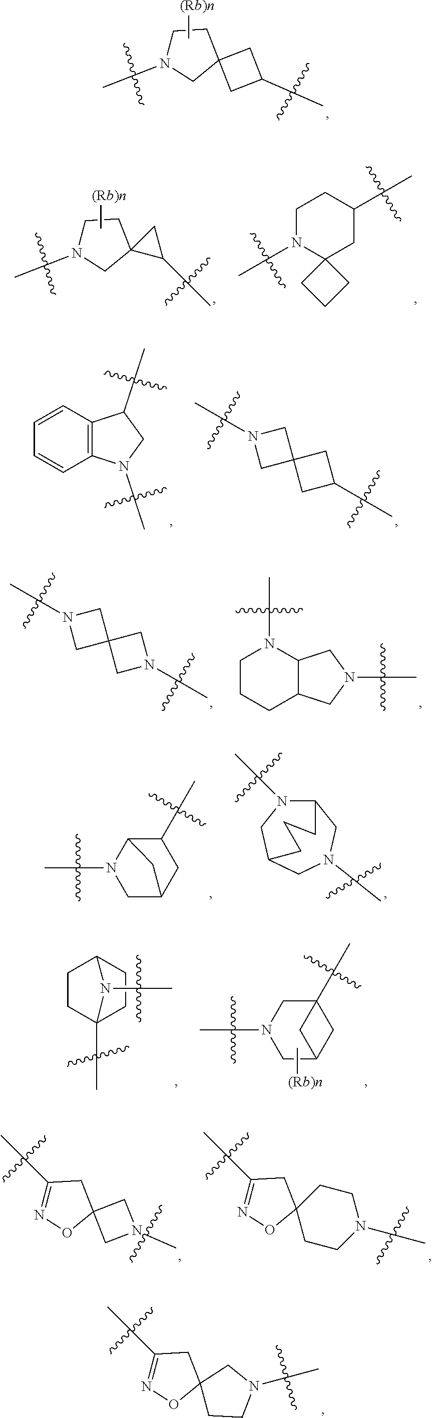

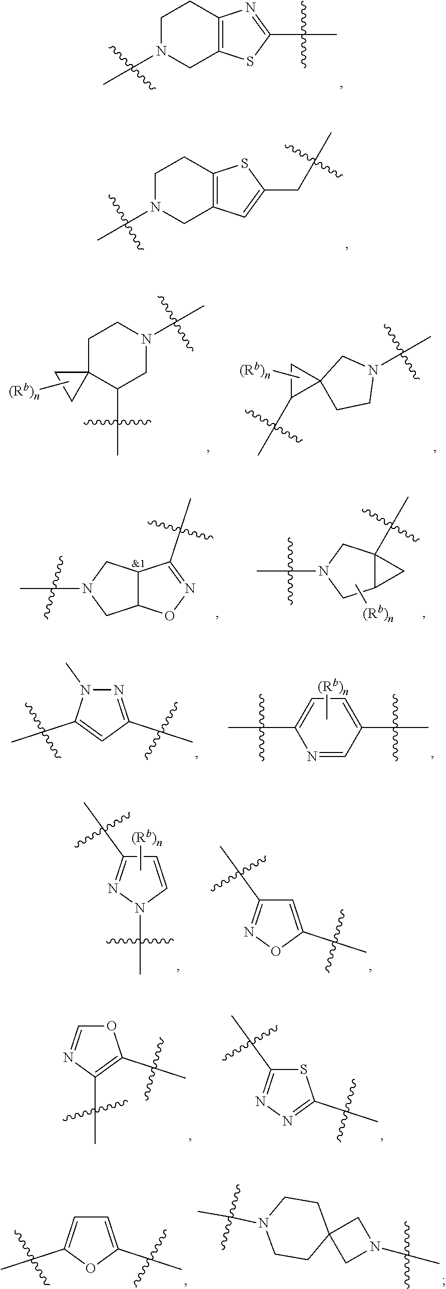

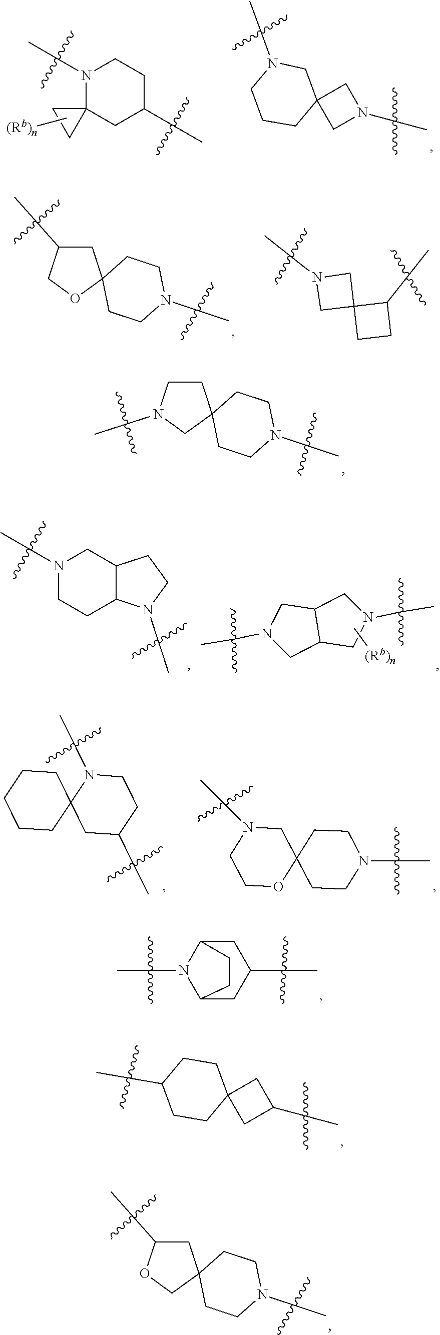

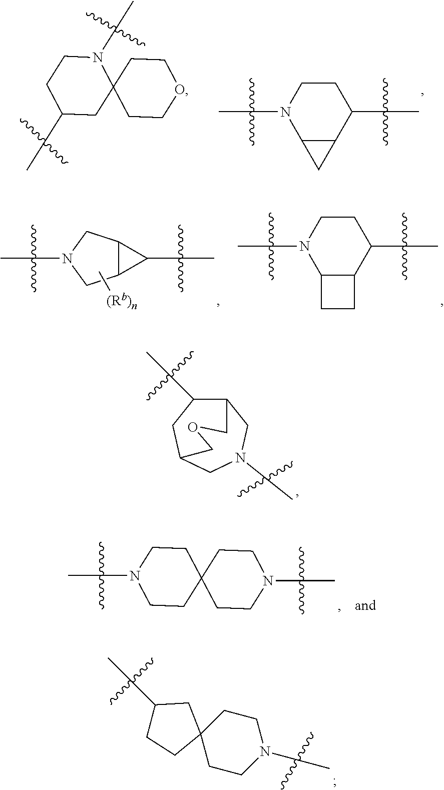

ii) direct bond;
iii) C₁₋₆ alkylene chain optionally substituted with 1-3 R^{b}; or
iv) —C(O)—, —O—, —(CH₂)ₘ—C(O)—N(R^{c})—, —(CH₂)ₘ—C(O)—, —(CH₂)ₘ—N(R^{c})—, —C(O)—N(R^{c})—(CH₂)ₘ—, or —NH—(CH₂)ₘ—C(O)—, —(CH₂)ₘ—S(O)₂NH—, m being 0, 1, 2 or 3;
   wherein,
n is 0, 1, or 2;
R^{b} is halo, —O—R^{c}, —CN, C₁₋₆ alkyl, or C₁₋₆ haloalkyl; and
R^{c} is hydrogen, C₁₋₆ alkyl, C₁₋₆ alkoxy, C₃₋₆ cycloalkyl, phenyl, or C₁₋₆ haloalkyl, wherein C₁₋₆ alkyl is optionally substituted with C₁₋₃ alkoxy, hydroxy, C₃₋₆ cycloalkyl, phenyl and amino.

In specific embodiments, L₆, L₇, or L₈ are each direct bond.

In more specific embodiments, L₁ is —C(O)— or —C(O)—N(R^{c})—(R^{c} is hydrogen, C₁₋₆ alkyl, C₁₋₆ alkoxy, C₃₋₆ cycloalkyl, or C₁₋₆ haloalkyl, wherein C₁₋₆ alkyl is optionally substituted with 1 or 2 C₁₋₃ alkoxy, hydroxy, C₃₋₆ cycloalkyl, phenyl or amino),
L₂ is

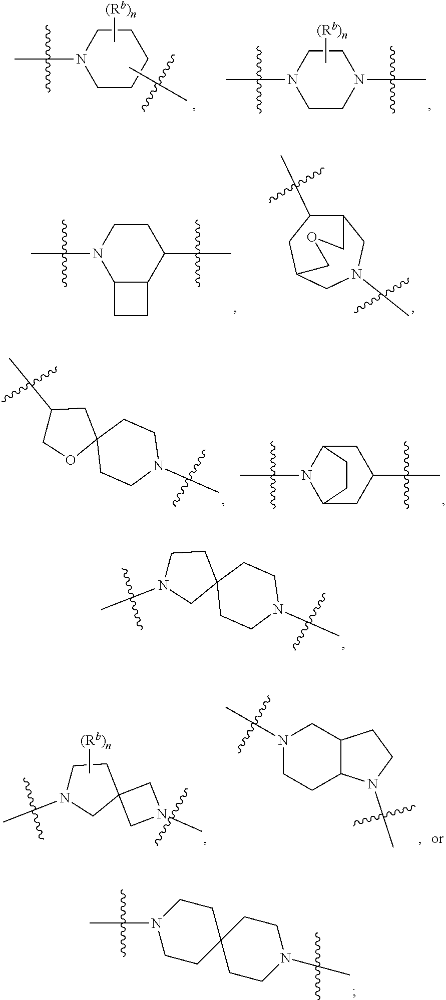

L₃ is direct bond, —CH₂—C(O)— or C₁₋₂ alkylene chain optionally substituted with C₁₋₃ alkyl;
L₄ is

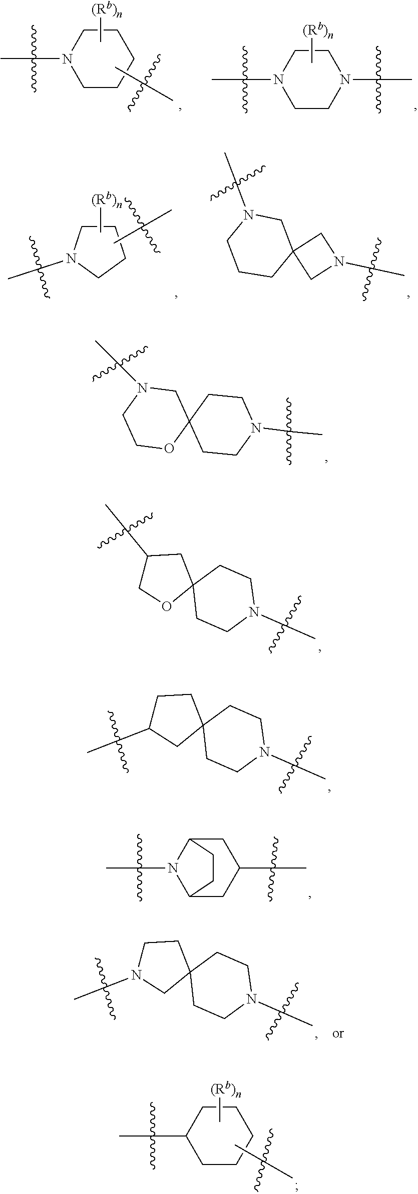

n is 0, 1 or 2, R^{b} is halo or C₁₋₃ alkyl;
L₅ is direct bond or —O—,

In further more specific embodiments, -L₂-L₃-L₄- has the following structures:

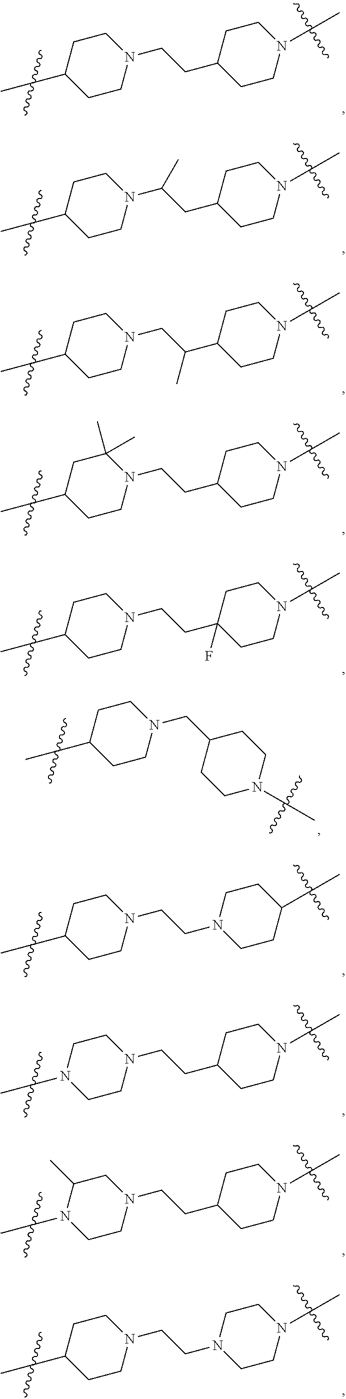

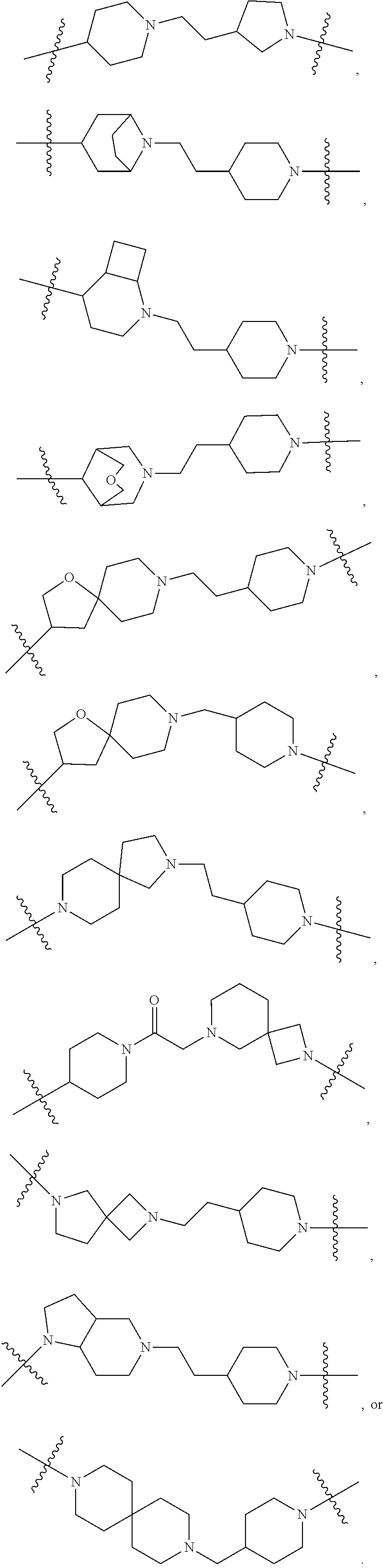

In yet other more specific embodiments, L₁ is —C(O)— or —C(O)—N(R^{c})—(R^{c} is hydrogen, C₁₋₆ alkyl, C₁₋₆ alkoxy, C₃₋₆ cycloalkyl, or C₁₋₆ haloalkyl, wherein C₁₋₆ alkyl is optionally substituted with 1 or 2 C₁₋₃ alkoxy, hydroxy, C₃₋₆ cycloalkyl, phenyl or amino),
L₃ is a direct bond, and
-L₂-L₃-L₄- has the following structures:

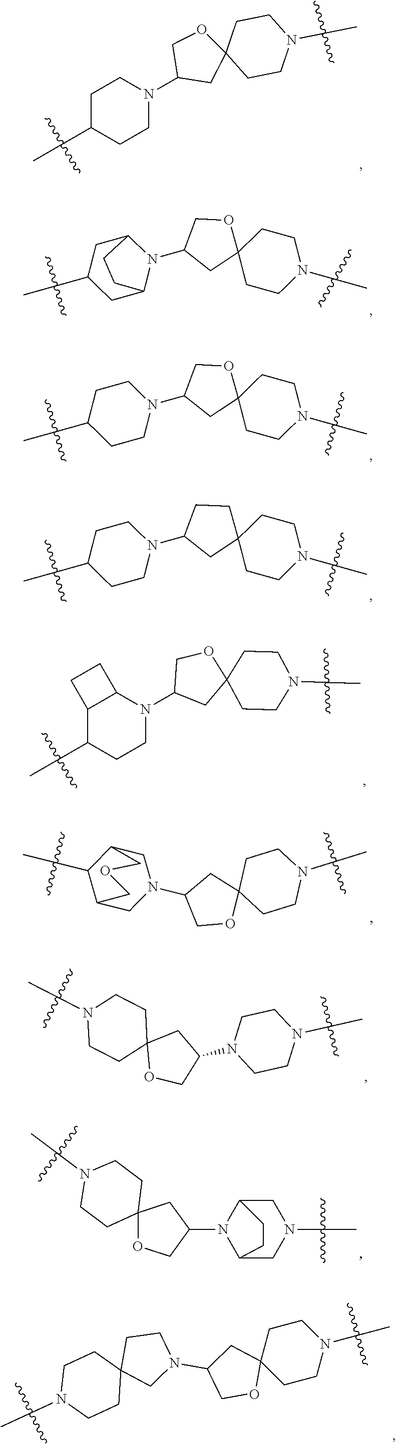

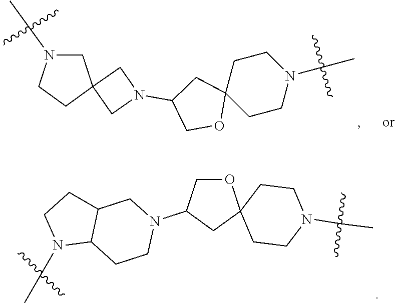 [SMILES: *N1CCC2(CC1)CC(N1CC3(CCN(*)C3)C1)CO2 ; *N1CCC2(CC1)CC(N1CCC3C(CCN3*)C1)CO2]

In other more specific embodiments, L₁ is —C(O)—N(R^{c})—, wherein R^{c} is C₁₋₆ alkyl, C₁₋₆ alkoxy, cyclopropyl (optionally substituted with methyl or methoxy), cyclobutyl (optionally substituted with 1 or 2 methyl or F), azetidinyl (optionally substituted with 1 or 2 methyl), pyrazolyl (optionally substituted with difluoromethyl), phenyl, or C₁₋₆ haloalkyl, wherein C₁₋₆ alkyl is optionally substituted with 1 or 2 C₁₋₃ alkoxy, CN, oxazolyl, thiazolyl (optionally substituted with methyl), pyrazinyl, pyrimidinyl, 1-methyl-1,2,3-triazolyl, pyrazolyl (optionally substituted with 1 or 2 C₁₋₃ alkyl), oxadiazolyl (optionally substituted with methyl), cyclopropyl (optionally substituted with C₁₋₃ alkoxy), cyclobutyl, or 3,3-difluorocyclobutyl);
-L₂-L₃-L₄-L₅- is

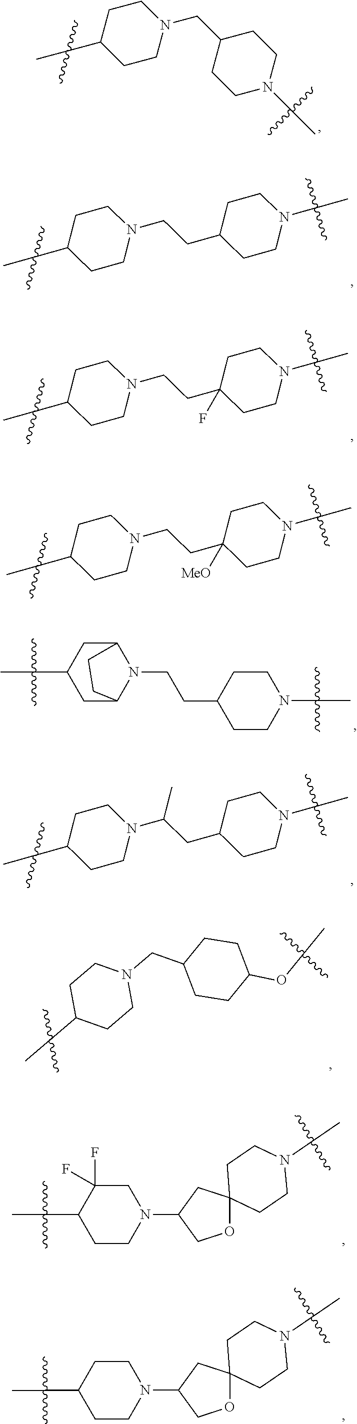

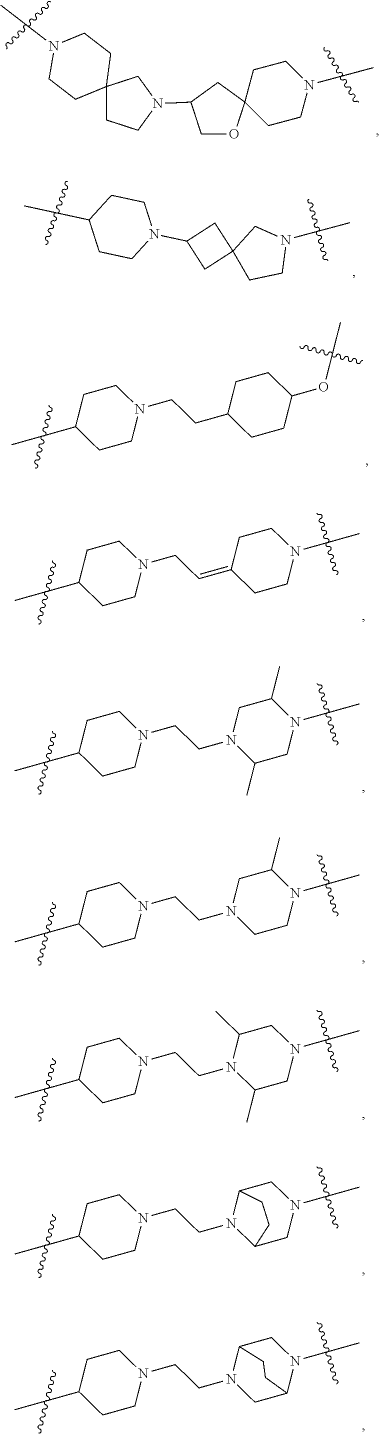

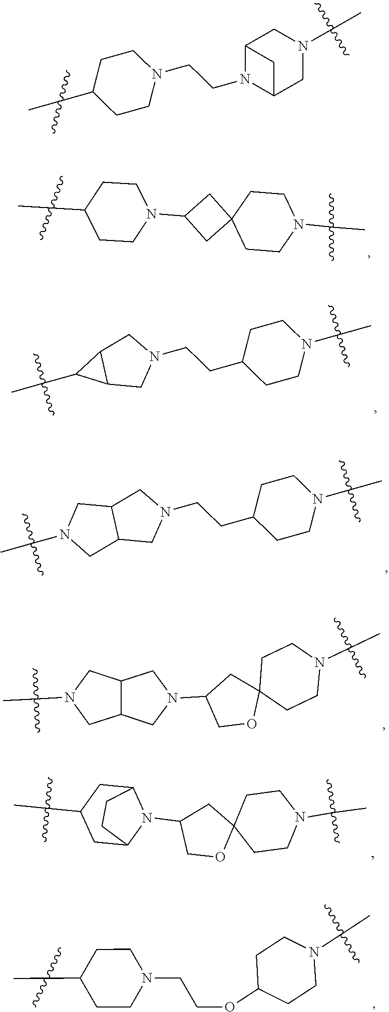

In yet other embodiments, L₁ is —C(O)—; and
-L₂-L₃-L₄-L₅- is

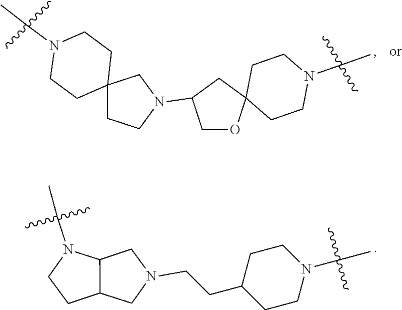 [SMILES: *N1CCC2(CC1)CCN(C1COC3(CCN(*)CC3)C1)C2 ; *N1CCC(CCN2CC3CCN(*)C3C2)CC1]

### Definitions

The following description sets forth exemplary methods, parameters and the like. It should be recognized, however, that such description is not intended as a limitation on the scope of the present disclosure but is instead provided as a description of exemplary embodiments.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

The prefix “Cᵤ₋ᵥ” indicates that the following group has from u to v carbon atoms. For example, “C₁₋₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain containing no unsaturation.

As used herein, alkyl has 1 to 20 carbon atoms (i.e., C₁₋₂₀ alkyl), 1 to 12 carbon atoms (i.e., C₁₋₁₂ alkyl), 1 to 8 carbon atoms (i.e., C₁₋₈ alkyl), 1 to 6 carbon atoms (i.e., C₁₋₆ alkyl), or 1 to 4 carbon atoms (i.e., C₁₋₄ alkyl). Examples of alkyl groups include methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e., —CH(CH₃)₂).

“Alkylene” or “alkylene chain” refers to a unbranched or branched divalent hydrocarbon chain, linking the rest of the molecule to a radical group, containing no unsaturation and having from 1 to 20 carbon atoms, or more typically 1 to 12 carbon atoms (C₁₋₁₂ alkylene), or 1 to 8 carbon atoms (C₁₋₈ alkylene), or 1 to 3 carbon atoms (C₁₋₃ alkylene) e.g., methylene, ethylene, propylene, n-butylene, and the like. The alkylene chain may be attached to the rest of the molecule and to the radical group through one carbon within the chain or through any two carbons within the chain.

“Alkenyl” refers to an alkyl group containing at least one carbon-carbon double bond and having from 2 to 20 carbon atoms (i.e., C₂₋₂₀ alkenyl), or more typically 2 to 12 carbon atoms (i.e., C₂₋₁₂ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include ethenyl, propenyl, butadienyl (including 1,2-butadienyl and 1,3-butadienyl).

“Alkenylene” and “alkenylene chain” refer to an unbranched or branched divalent hydrocarbon chain linking the rest of the molecule to a radical group, containing at least one double bond and having from 2 to carbon atoms, or more typically 2 to 12 carbon atoms, or 2 to 8 carbon atoms, e.g., ethenylene, propenylene, n-butenylene, and the like. The alkenylene chain is attached to the rest of the molecule through a single bond and to the radical group through a double bond or a single bond. The points of attachment of the alkenylene chain to the rest of the molecule and to the radical group can be through one carbon or any two carbons within the chain.

“Alkynyl” refers to an alkyl group containing at least one carbon-carbon triple bond and having from 2 to 20 carbon atoms (i.e., C₂₋₂₀ alkynyl), or more typically 2 to 12 carbon atoms (i.e., C₂₋₁₂ alkynyl), or more typically 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

“Alkynylene” and “alkynylene chain” refer to a unbranched or branched divalent hydrocarbon chain linking the rest of the molecule to a radical group, containing at least one triple bond and having from 2 to 20 carbon atoms, or more typically 2 to 12 carbon atoms, or 2 to 8 carbon atoms. The alkynylene chain is attached to the rest of the molecule through a single bond and to the radical group through a double bond or a single bond. The points of attachment of the alkynylene chain to the rest of the molecule and to the radical group can be through one carbon or any two carbons within the chain.

“Alkoxy” refers to the group “alkyl-O—”. Examples of alkoxy groups include methoxy, ethoxy, n-propoxy, iso-propoxy, n-butoxy, tert-butoxy, sec-butoxy, n-pentoxy, n-hexoxy, and 1,2-dimethylbutoxy.

“Haloalkoxy” refers to an alkoxy group as defined above, wherein one or more hydrogen atoms are replaced by a halogen.

“Alkylthio” refers to the group “alkyl-S—”.

“Amino” refers to the group —NR^{y}R^{y} wherein each R^{y} is independently selected from the group consisting of hydrogen, alkyl, alkenyl, alkynyl, aryl, heterocyclyl, cycloalkyl or heteroaryl, each of which is optionally substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆₋₂₀ aryl), 6 to 15 carbon ring atoms (i.e., C₆₋₁₅ aryl), or 6 to 10 carbon ring atoms (i.e., C₆₋₁₀ aryl). Examples of aryl groups include phenyl, naphthyl, fluorenyl, and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl.

“Cyano” refers to the group —CN.

“Keto” or “oxo” refers to a group ═O.

“Carbamoyl” refers to both an “O-carbamoyl” group which refers to the group —O—C(O)NR^{y}R^{z} and an “N-carbamoyl” group which refers to the group —NR^{y}C(O)OR^{z}, wherein R^{y} and R^{z} are independently selected from the group consisting of hydrogen, alkyl, aryl, haloalkyl, or heteroaryl; each of which may be optionally substituted.

“Carboxyl” or “carboxylic acid” refers to —C(O)OH.

“Ester” refers to both —OC(O)R and —C(O)OR, wherein R is a substituent; each of which may be optionally substituted, as defined herein.

“Cycloalkyl” refers to a saturated or partially unsaturated cyclic alkyl group having a single ring or multiple rings including fused, bridged, and spiro ring systems. The term “cycloalkyl” includes cycloalkenyl groups (i.e., the cyclic group having at least one double bond). As used herein, cycloalkyl has from 3 to 15 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include cyclopropyl, cyclobutyl, cyclopentyl, cyclohexyl, and bicyclo[2.2.2]octan-1-yl. Cycloalkyl may be attached to the remainder of a molecule by a single ring atom (e.g., as a substituent) or by two ring atoms (e.g., as a linker).

“Ethylene glycol unit” refers to a bivalent monomer having the structure of —CH₂CH₂O—, which may be repeated and extended into a longer chain. A linker segment may have up to 12 ethylene glycol units, or more typically up to 6 ethylene glycol units.

“Propylene glycol unit” refers to a bivalent monomer having the structure of —CH(CH₃)—CH₂O—, which may be repeated and extended into a longer chain. A linker segment may have up to 12 propylene glycol units, or more typically up to 6 propylene glycol units.

“Halogen” or “halo” includes fluoro, chloro, bromo, and iodo.

“Haloalkyl” refers to an unbranched or branched alkyl group as defined above, wherein one or more hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include difluoromethyl (—CHF₂) and trifluoromethyl (—CF₃).

“Heteroalkyl” refers to an alkyl group in which one or more of the carbon atoms (and any associated hydrogen atoms) are each independently replaced with the same or different heteroatoms such as N, O, S, and the likes. The term “heteroalkyl” includes unbranched or branched saturated chain having carbon and heteroatoms. By way of example, 1, 2 or 3 carbon atoms may be independently replaced with the same or different heteroatoms. Heteroatomic groups include, but are not limited to, —N(R)—, —O—, —S—, —S(O)—, —S(O)₂—, and the like, where R is H, alkyl, aryl, cycloalkyl, heteroalkyl, heteroaryl or heterocyclyl, each of which may be optionally substituted. Examples of heteroalkyl groups include —OCH₃, —CH₂OCH₃, —SCH₃, —CH₂SCH₃, —NRCH₃, and —CH₂NRCH₃, where R is hydrogen, alkyl, aryl, arylalkyl, heteroalkyl, or heteroaryl, each of which may be optionally substituted. As used herein, heteroalkyl include 1 to 10 carbon atoms, 1 to 8 carbon atoms, or 1 to 4 carbon atoms; and 1 to 3 heteroatoms, 1 to 2 heteroatoms, or 1 heteroatom.

“Heteroaryl” refers to a 5-15 membered, or more typically, 5-12 membered aromatic group having a single ring, multiple rings, or multiple fused rings, with 1-3 ring heteroatoms independently selected from nitrogen, oxygen, and sulfur. As used herein, heteroaryl includes 3 to 12 ring carbon atoms (i.e., C₃₋₁₂ heteroaryl), or 3 to 8 carbon ring atoms (i.e., C₃₋₈ heteroaryl); and 1 to 5 heteroatoms, 1 to 4 heteroatoms, 1 to 3 ring heteroatoms, 1 to 2 ring heteroatoms, or 1 ring heteroatom independently selected from nitrogen, oxygen, and sulfur. Examples of heteroaryl groups include pyrimidinyl, purinyl, pyridyl, pyridazinyl, benzothiazolyl, imidazoyl, thiazolyl, oxazolyl, triazolyl, pyrazinyl, isoxazolyl and pyrazolyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl (which has no heteroatom) or heterocyclyl (which has at least one non-aromatic ring). Heteroaryl may be attached to the remainder of a molecule by any single ring atom (e.g., as a substituent) or by any two ring atoms (e.g., as a linker), provided that the valence is satisfied.

“Heterocyclyl” refers to a 3-15 membered, or more typically, 5-12 membered, saturated or unsaturated cyclic alkyl group, with 1-3 ring heteroatoms independently selected from nitrogen, oxygen and sulfur. The term “heterocyclyl” includes heterocycloalkenyl groups (i.e., the heterocyclyl group having at least one double bond), bicyclic heterocyclyl groups, bridged-heterocyclyl groups, fused-heterocyclyl groups, and spiro-heterocyclyl groups. A heterocyclyl may be a single ring or multiple rings wherein the multiple rings may be fused, bridged, or spiro. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 3 to 15 ring atoms (e.g., 3-15 membered heterocyclyl, 3-12 membered heterocyclyl, 4 to 10 membered heterocyclyl, 4-8 membered heterocyclyl or 4-6 membered heterocyclyl; having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. A heterocyclyl may contain one or more oxo and/or thioxo groups. Examples of heterocyclyl groups include pyrrolidinyl, piperidinyl, piperazinyl, oxetanyl, dioxolanyl, azetidinyl, azetidinyl, morpholinyl, thiomorpholinyl, 4-7 membered sultam, 4-7 membered cyclic carbamate, 4-7 membered cyclic carbonate, 4-7 membered cyclic sulfide and morpholinyl. As used herein, heterocyclyl may include a bridged structure (i.e., “bridged heterocyclyl), in which a four- to ten-membered cyclic moiety connected at two non-adjacent atoms of the heterocyclyl with one or more (e.g., 1 or 2) four- to ten-membered cyclic moiety having at least one heteroatom where each heteroatom is independently selected from nitrogen, oxygen, and sulfur. As used herein, bridged-heterocyclyl includes bicyclic and tricyclic ring systems. Also used herein, the term “spiro-heterocyclyl” refers to a ring system in which a three- to ten-membered heterocyclyl has one or more additional ring, wherein the one or more additional ring is three- to ten-membered cycloalkyl or three- to ten-membered heterocyclyl, where a single atom of the one or more additional ring is also an atom of the three- to ten-membered heterocyclyl. Examples of the spiro-heterocyclyl rings include bicyclic and tricyclic ring systems, such as 2-oxa-7-azaspiro[3.5]nonanyl, 2-oxa-6-azaspiro[3.4]octanyl, and 6-oxa-1-azaspiro[3.3]heptanyl. Examples of the fused-heterocyclyl rings include, but are not limited to, 1,2,3,4-tetrahydroisoquinolinyl, 1-oxo-1,2,3,4-tetrahydroisoquinolinyl, 1-oxo-1,2-dihydroisoquinolinyl, 4,5,6,7-tetrahydrothieno[2,3-c]pyridinyl, indolinyl, and isoindolinyl, where the heterocyclyl can be bound via either ring of the fused system. As used herein, a bicyclic heterocyclyl group is a heterocyclyl group attached at two points to another cyclic group, wherein the other cyclic group may itself be a heterocyclic group, or a carbocyclic group. Heteroaryl may be attached to the remainder of a molecule by a single ring atom (e.g., as a substituent) or by two ring atoms (e.g., as a linker).

“Fused” refers to a ring which is joint to an adjacent ring and share two adjacent ring atoms that form a covalent bond.

“Bridged” refers to a ring fusion wherein non-adjacent atoms on a ring are joined by a divalent substituent, such as alkylenyl group, an alkylenyl group containing one or two heteroatoms, or a single heteroatom. Quinuclidinyl and admantanyl are examples of bridged ring systems.

“Spiro” refers to a ring substituent which is joined by two bonds at the same carbon atom. Examples of spiro groups include 1,1-diethylcyclopentane, dimethyl-dioxolane, and 4-benzyl-4-methylpiperidine, wherein the cyclopentane and piperidine, respectively, are the spiro substituents.

“Hydroxy” or “hydroxyl” refers to the group —OH. “Hydroxyalkyl” refers to an unbranched or branched alkyl group as defined above, wherein one or more hydrogen atoms are replaced by a hydroxyl.

“Nitro” refers to the group —NO₂.

“Imino” refers to a group that contains a C═N double bond, such as C═N—R^{y}, or =N—C(O)R^{y}, wherein R^{y} is selected from the group consisting of hydrogen, alkyl, aryl, cyano, haloalkyl, or heteroaryl; each of which may be optionally substituted. Imino may be a linker segment by attaching to the remainder molecule at the carbon and nitrogen respectively.

“Sulfonyl” refers to the group —S(O)₂R, where R is a substituent, or a defined group.

“Alkylsulfonyl” refers to the group —S(O)₂R, where R is a substituent, or a defined group.

“Alkylsulfinyl” refers to the group —S(O)R, where R is a substituent, or a defined group.

“Thiocyanate” —SCN.

“Thiol” refers to the group —SR, where R is a substituent, or a defined group.

“Thioxo” or “thione” refer to the group (=S) or (S).

Certain commonly used alternative chemical names may be used. For example, a divalent group such as a divalent “alkyl” group, a divalent “aryl” group, etc., may also be referred to as an “alkylene” group or an “alkylenyl” group, an “arylene” group or an “arylenyl” group, respectively. Also, unless indicated explicitly otherwise, where combinations of groups are referred to herein as one moiety, e.g., arylalkyl, the last mentioned group contains the atom by which the moiety is attached to the rest of the molecule.

The terms “optional” or “optionally” means that the subsequently described event or circumstance may or may not occur, and that the description includes instances where said event or circumstance occurs and instances in which it does not. Also, the term “optionally substituted” refers to any one or more hydrogen atoms on the designated atom or group may or may not be replaced by a moiety other than hydrogen. “Optionally substituted” may be zero to the maximum number of possible substitutions, and each occurrence is independent. When the term “substituted” is used, then that substitution is required to be made at a substitutable hydrogen atom of the indicated substituent. An optional substitution may be the same or different from a (required) substitution.

When a moiety is “optionally substituted,” and reference is made to a general term, such as any “alkyl,” “alkenyl,” “alkynyl,” “haloalkyl,” “cycloalkyl,” “aryl” or “heteroaryl,” then the general term can refer to any antecedent specifically recited term, such as (C₁₋₃ alkyl), (C₄₋₆ alkyl), —O(C₁₋₄ alkyl), (C₃₋₁₀ cycloalkyl), O—(C₃₋₁₀ cycloalkyl) and the like. For example, “any aryl” includes both “aryl” and “—O(aryl) as well as examples of aryl, such as phenyl or naphthyl and the like. Also, the term “any heterocyclyl” includes both the terms “heterocyclyl” and O-(heterocyclyl),” as well as examples of heterocyclyls, such as oxetanyl, tetrahydropyranyl, morpholino, piperidinyl and the like. In the same manner, the term “any heteroaryl” includes the terms “heteroaryl” and “O-(heteroryl),” as well as specific heteroaryls, such as pyridine and the like.

Some compounds of Formula (I) may exist as a “stereoisomer” or a mixture of stereoisomers. Stereoisomer refers to a compound made up of the same atoms bonded by the same bonds but having different three-dimensional structures, which are not interchangeable. The compounds of the disclosure, or their pharmaceutically acceptable salts may contain one or more asymmetric centers and may thus give rise to enantiomers (two stereoisomers whose molecules are non-superimposable mirror images of one another), diastereomers, and other stereoisomeric forms that may be defined, in terms of absolute stereochemistry, as (R)- or (S)-. The present disclosure is meant to include all such possible isomers, as well as their racemic mixture (i.e., equal amounts of (R) and (S) enantiomers) and optically pure forms. Optically active (+) and (−), (R)- and (S)-isomers may be prepared using chiral synthons or chiral reagents, or resolved using conventional techniques, such as HPLC or SFC using a chiral column.

“& 1” means that a compound including the “&1” notation at a particular chemical element or atom (e.g., carbon) within the compound was prepared as a mixture of two stereoisomers at the noted chemical element or atom (e.g., a diastereomeric mixture having a de or % de as described above). “&2,” if presents, denotes a second set of isomers.

In many cases, the compounds of this disclosure are capable of forming acid and/or base salts by virtue of the presence of amino and/or carboxyl groups or groups similar thereto. Provided are pharmaceutically acceptable salts, hydrates, or solvates of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical uses.

The term “pharmaceutically acceptable salt” of a given compound refers to salts that retain the biological effectiveness and properties of the given compound, and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃), or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

The term “substituted” means that any one or more hydrogen atoms on the designated atom or group is replaced with one or more substituents other than hydrogen, provided that the designated atom's normal valence is not exceeded. The one or more substituents include, but are not limited to, alkyl, alkenyl, alkynyl, alkoxy, acyl, amino, amido, amidino, aryl, azido, carbamoyl, carboxyl, carboxyl ester, cyano, guanidino, halo, haloalkyl, haloalkoxy, heteroalkyl, heteroaryl, heterocyclyl, hydroxy, hydrazino, imino, oxo, nitro, alkylsulfinyl, sulfonic acid, alkylsulfonyl, thiocyanate, thiol, thione, or combinations thereof. Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein. Unless specified otherwise, where a group is described as optionally substituted, any substituents of the group are themselves unsubstituted. For example, in some embodiments, the term “substituted alkyl” refers to an alkyl group having one or more substituents including hydroxyl, halo, alkoxy, cycloalkyl, heterocyclyl, aryl, and heteroaryl. In other embodiments, the one or more substituents may be further substituted with halo, alkyl, haloalkyl, hydroxyl, alkoxy, cycloalkyl, heterocyclyl, aryl, or heteroaryl, each of which is substituted. In other embodiments, the substituents may be further substituted with halo, alkyl, haloalkyl, alkoxy, hydroxyl, cycloalkyl, heterocyclyl, aryl, or heteroaryl, each of which is unsubstituted One skilled in the art will recognize that substituents and other moieties of the compounds of the generic formula herein should be selected in order to provide a compound which is sufficiently stable to provide a pharmaceutically useful compound which can be formulated into an acceptably stable pharmaceutical composition. Compounds which have such stability are contemplated as falling within the scope of the present invention. It should be understood by one skilled in the art that any combination of the definitions and substituents described above should not result in an inoperable species or compound.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

A “solvate” is formed by the interaction of a solvent and a compound. Solvates of salts of the compounds described herein are also provided. Hydrates of the compounds described herein are also provided.

### Targeted SMARCA2 Degradation

The compounds of the present disclosure are demonstrated by cell-based profiling to selectively degrade SMARCA2 while sparing SMARCA4.

Additional biological activities, including selectivity data of compounds of Formula (I) are summarized in Table 1 herein.

### Pharmaceutical Composition and Use of the Bifunctional Compounds of Formula (I)

The bifunctional compounds of Formula (I) or Formula (II) are demonstrated to selectively degrade SMARCA2 and are therefore particularly useful for treating SMARCA4-deficient cancers.

Various embodiments provide pharmaceutical compositions of a compound of Formula (I), or any one of the substructures or specific compounds of Examples 1-263, and a pharmaceutically acceptable carrier.

Further embodiments provide methods for treating SMARCA2-mediated disease or disorder, including, for increasing T-cell activation, for treating cancer, for inhibiting the growth or proliferation of cancer cells, the method comprising administering to a subject in need thereof a therapeutically effective amount of a compound of Formula (I) or Formula (II), any one of the substructures or compounds of Examples 1-263.

SMARCA2-mediated diseases may be cancers selected from the group consisting of acoustic neuroma, acute leukemiat, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, liver cancer, lung cancer, lymphagioendotheliosarcoma, lymphangiosarcoma, lymphoblastic leukemia, lymphoma (Hodgkin's and non-Hodgkin's; Burkitt's), malignancies and hyperproliferative disorders of the bladder, breast, colon, lung, ovaries, pancreas, prostate, skin and uterus, lymphoid malignancies of T-cell or B-cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, malignant rhabdoid tumor (MRT), rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

In some embodiments, a compound of Formula (I), or any one of the substructures or a compound of Examples 1-263 may be co-administered with a therapeutically effective amount of one or more additional therapeutic agents, or a pharmaceutically acceptable salt thereof. The additional therapeutic agents include, for example, chemotherapeutic agents disclosed in WO WO2021083949.

### Construction of Compounds of Formula (I)

The synthesis or construction of the compounds of Formula (I) can be carried out in multiple steps, typically involving separately preparing building blocks of the SMARCA2 binder and the LHM moiety, followed by joining the respective building blocks through covalent bond formation. Generally speaking, either or both building blocks may be prepared with one or more linker precursors. A linker precursor comprises one or more linker segments (Lₛ) and has a terminal reactive group for further coupling. The two building blocks can be finally coupled (via formation of a further linker segment) to afford a compound of Formula (I).

The following schemes demonstrate the general approaches of preparing building blocks. Specific examples (Examples 1-263) were synthesized and characterized by their respective physiochemical properties according to the general schemes described herein.

### INTERMEDIATES

### Intermediate 1

### LITHIUM 4-METHOXY-1-(6-(2-(METHOXYMETHOXY)PHENYL)PYRIDAZIN-4-YL)PIPERIDINE-4-CARBOXYLATE

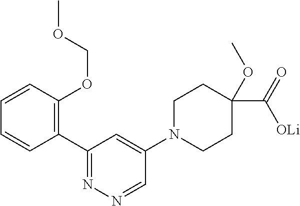

### Step 1: Synthesis of 1-(tert-butyl) 4-methyl 4-methoxypiperidine-1,4-dicarboxylate

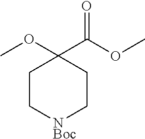

A flask was charged with 4.63 g of NaH (60% dispersion in oil) and the solid was washed with hexanes 2×. For washing procedure, add hexanes and swirl flask. Let flask sit for 2 mins and then decant hexanes using syringe 2×. Once complete, DMF (30 mL) was added to the flask under N₂ and the mixture was cooled to 0° C. in an ice bath. 1-tert-butyl 4-methyl 4-hydroxypiperidine-1,4-dicarboxylate (3.00 g, 11.5 mmol) was dissolved in 15 mL DMF and the solution was added slowly to the solution of NaH in DMF. Once complete, the reaction was allowed to stir for 30 mins. Methyl iodide (7.20 mL, 16.4 g, 115 mmol) was then added over 10 mins slowly to the reaction mixture at 0-5° C. Slight bubbling was observed. Once complete, the reaction was stirred in the melted ice bath for 2 hours exactly. TLC of the reaction mixture (3:7 EA:Hex, ninhydrin stain) indicates all SM is consumed and clear conversion to product. The reaction was chilled in a new ice bath at ° C. To prevent hydrolysis of ester during quench, 3 mL of MeOH was slowly added to the reaction mixture and H₂ gas evolved. Then, sat. NH₄Cl was added slowly. The mixture was transferred to a separatory funnel and extracted 3× with EtOAc. The organic phases were combined, dried and concentrated in vacuo. The residue was purified via chromatography (EA:Hex gradient) to yield the desired product (2.82 g, 89% yield) as a clear oil. LCMS: C₁₃H₂₃NO₅ requires: 273.1, found: m/z=274.4 [M+H]⁺

### Step 2: Synthesis of methyl 4-methoxypiperidine-4-carboxylate hydrochloride

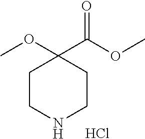

1-tert-butyl 4-methyl 4-methoxypiperidine-1,4-dicarboxylate (3.88 g, 14.1 mmol) was dissolved in 4M HCl in dioxane (25.00 mL) and stirred at RT. After 1 hr, the reaction was checked by LCMS and solid has precipitated out. Once complete, the reaction is concentrated in vacuo to give a white powder. The resulting solid is dried further on a high vacuum system to yield the product (2.88 g, 96% yield) as a while solid. LCMS: C₈H₁₅NO₃ requires: 173.1, found: m/z=174.3 [M+H]⁺

### Step 3: Synthesis of methyl 1-(6-chloropyridazin-4-yl)-4-methoxypiperidine-4-carboxylate

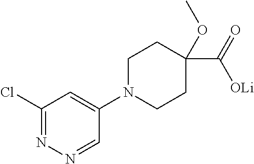

Methyl 4-methoxypiperidine-4-carboxylate hydrochloride (2.88 g, 13.7 mmol) and 3,5-dichloropyridazine (2.05 g, 13.7 mmol) were dissolved in DMSO (30 mL) under argon atmosphere. N,N-diisopropylethylamine (12.0 mL, 8.88 g, 68.6 mmol) was then added and the resulting solution was stirred at 100° C. for 12 hours. The reaction was monitored via LCMS. Once complete, the reaction was concentrated in vacuo to remove excess DIPEA and then quenched with H₂O and extracted with EtOAc 3 times. The organic phases were combined, dried and concentrated in vacuo. The residue was purified via chromatography (EA:hexane, gradient) to produce a red oil. Diethyl ether (10 mL) was added to the red oil and a pink solid was precipitated out. The solid was collected and further dried to yield the product (3.21 g, 81% yield) as a pink solid. LCMS: C₁₂H₁₆ClN₃O₃ requires: 285.0, found: m/z=285.3 [M+H]⁺

### Step 4: Synthesis of methyl 4-methoxy-1-(6-(2-(methoxymethoxy)phenyl)pyridazin-4-yl)piperidine-4-carboxylate

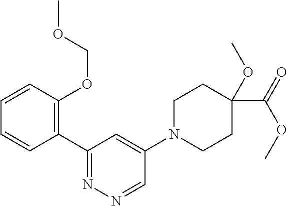 [SMILES: COCOc1ccccc1-c1cc(N2CCC(OC)(C(=O)OC)CC2)cnn1]

A flask was charged with methyl 1-(6-chloropyridazin-4-yl)-4-methoxypiperidine-4-carboxylate (850 mg, 2.97 mmol), potassium carbonate (1.23 g, 8.92 mmol), and 2-(methoxymethoxy)phenylboronic acid (812 mg, 4.46 mmol) under N₂ atmosphere. 1,4-dioxane (12.00 mL) and water (3.00 mL) were sequentially added to the flask. The resulting solution was degassed with an Argon balloon for 15 mins and then tetrakis(triphenylphosphine)palladium(O) (343 mg, 0.297 mmol) was added in one portion. The flask was sealed and the resulting mixture was stirred at 100° C. for 12 h. Upon completion, the flask was diluted in EtOAc and sat. NH₄Cl was added. The mixture was extracted 3× with EtOAc and the organic phases were concentrated in vacuo. The residue was purified via chromatography (EA:hexane, gradient) to give the desired product (0.512 g, 44% yield) as an orange oil. LCMS: C₂₀H₂₅N₃O₅ requires: 387.1, found: m/z=387.4 [M+H]⁺

### Step 5: Synthesis of Title Compound

methyl 4-methoxy-1-{6-[2-(methoxymethoxy)phenyl]pyridazin-4-yl}piperidine-4-carboxylate (512 mg, 1.32 mmol) was dissolved in 6 mL of a 1:1 THF/H₂O mixture and lithium hydroxide monohydrate (55.4 mg, 1.32 mmol) was added in one portion. The reaction was stirred at RT until complete by LCMS. Once complete, the reaction mixture is concentrated in vacuo and lyophilized to afford the desired lithium salt (510 mg, 101% yield) as a yellow powder. LCMS: C₁₉H₂₂N₃O₅ requires: 372.1, found: m/z=373.1 [M+H]⁺

### Intermediate 2

### LITHIUM 1-(6-(5-FLUORO-2-OXIDOPHENYL)PYRIDAZIN-4-YL)-4-METHOXYPIPERIDINE-4-CARBOXYLATE

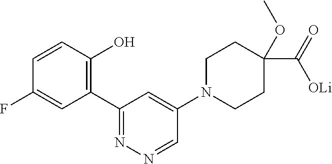

### Step 1: Synthesis of methyl 1-(6-(5-fluoro-2-hydroxyphenyl)pyridazin-4-yl)-4-methoxypiperidine-4-carboxylate

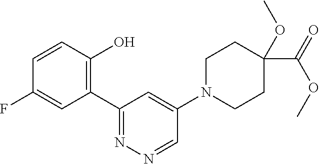 [SMILES: COC(=O)C1(OC)CCN(c2cnnc(-c3cc(F)ccc3O)c2)CC1]

A flask was charged with methyl 1-(6-chloropyridazin-4-yl)-4-methoxypiperidine-4-carboxylate (500 mg, 1.74 mmol), potassium carbonate (725 mg, 5.24 mmol), and 5-fluoro-2-hydroxyphenylboronic acid (409 mg, 2.62 mmol). 1,4-dioxane (9.00 mL) and water (2.00 mL) were sequentially added to the flask. The resulting solution was degassed with an Argon balloon for 5 mins and then tetrakis(triphenylphosphine)palladium(O) (202 mg, 0.175 mmol) was added in one portion. The flask was sealed and the resulting mixture was stirred at 100° C. for 18 h. Upon completion, the flask was diluted in EtOAc and sat. NH₄Cl was added. The mixture was extracted 3× with EtOAc and the organic phases were concentrated in vacuo. The residue was purified via chromatography (EA:hexane, gradient) to give the desired product (0.503 g, 79% yield) as an orange oil. LCMS: C₁₈H₂₀FN₃O₄ requires: 361.1, found: m/z=362.2 [M+H]⁺

### Step 2: Synthesis of the Title Compound

methyl 1-[6-(5-fluoro-2-hydroxyphenyl)pyridazin-4-yl]-4-methoxypiperidine-4-carboxylate (210 mg, 0.581 mmol) was dissolved in THF:H₂O (5 mL:5 mL) and lithium hydroxide monohydrate (48.7 mg, 1.16 mmol) was added in one portion. The reaction was stirred at RT until complete by LCMS. Once complete, the reaction mixture was concentrated in vacuo and lyophilized to afford the desired di-lithium salt (241 mg, 115%). LCMS: C₁₇H₁₆FN₃O₄ requires: 345.1, found: m/z=346.1 [M+H]⁺

### Intermediate 3

### LITHIUM 1-(6-(3-FLUORO-2-OXIDOPHENYL)PYRIDAZIN-4-YL)-4-METHOXYPIPERIDINE-4-CARBOXYLATE

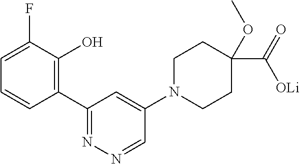

### Step 1: Synthesis of methyl 1-(6-(3-fluoro-2-hydroxyphenyl)pyridazin-4-yl)-4-methoxypiperidine-4-carboxylate

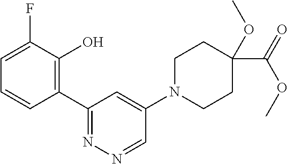 [SMILES: COC(=O)C1(OC)CCN(c2cnnc(-c3cccc(F)c3O)c2)CC1]

A flask was charged with methyl 1-(6-chloropyridazin-4-yl)-4-methoxypiperidine-4-carboxylate (500 mg, 1.74 mmol), potassium carbonate (725 mg, 5.24 mmol), and 3-fluoro-2-hydroxyphenylboronic acid (409 mg, 2.62 mmol). 1,4-dioxane (9.00 mL) and water (2.00 mL) were sequentially added to the flask. The resulting solution was degassed with an Argon balloon for 5 mins and then tetrakis(triphenylphosphine)palladium(O) (202 mg, 0.175 mmol) was added in one portion. The flask was sealed and the resulting mixture was stirred at 100° C. for 18 h. Upon completion, the flask was diluted in EtOAc and sat. NH₄Cl was added. The mixture was extracted 3× with EtOAc and the organic phases were concentrated in vacuo. The residue was purified via chromatography (EA:hexane, gradient) to give the desired product (0.305 g, 48% yield) as an orange oil. LCMS: C₁₈H₂₀FN₃O₄ requires: 361.1, found: m/z=362.2 [M+H]⁺

### Step 2: Synthesis of the Title Compound

Methyl 1-[6-(3-fluoro-2-hydroxyphenyl)pyridazin-4-yl]-4-methoxypiperidine-4-carboxylate (287 mg, 0.794 mmol) was dissolved in THF:H₂O (5 mL:5 mL) and lithium hydroxide monohydrate (66.6 mg, 1.58 mmol) was added in one portion. The reaction was stirred at RT until complete by LCMS. Once complete, the reaction mixture was concentrated in vacuo and lyophilized to afford the desired di-lithium salt (312 mg, 109%). LCMS: C₁₇H₁₆FN₃O₄ requires: 345.1, found: m/z=346.1 [M+H]⁺

### Intermediate 4

### LITHIUM 4-ETHOXY-1-(6-(2-OXIDOPHENYL)PYRIDAZIN-4-YL)PIPERIDINE-4-CARBOXYLATE

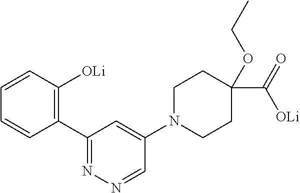

### Step 1: Synthesis of 1-(tert-butyl) 4-methyl 4-ethoxypiperidine-1,4-dicarboxylate

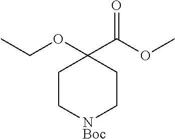 [SMILES: CCOC1(C(=O)OC)CCN(C(=O)OC(C)(C)C)CC1]

A flask was charged with 1.55 g of NaH (60% dispersion in oil) and the solid was washed with hexanes 2×. For washing procedure, add hexanes and swirl flask. Let flask sit for 2 mins and then decant hexanes using syringe 2×. Once complete, DMF (5 mL) was added to the flask under N₂ and the mixture was cooled to 0° C. in an ice bath. 1-tert-butyl 4-methyl 4-hydroxypiperidine-1,4-dicarboxylate (1.00 g, 3.85 mmol) was dissolved in 10 mL DMF and the solution was added slowly to the solution of NaH in DMF. Once complete, the reaction was allowed to stir for 30 mins. Ethyl iodide (3.10 mL, 6.01 g, 38.5 mmol) was then added over 10 mins slowly to the reaction mixture at 0-5° C. Slight bubbling was observed. Once complete, the reaction was stirred in the melted ice bath for 2 hours exactly. TLC of the reaction mixture (3:7 EA:Hex, ninhydrin stain) indicates all SM is consumed and clear conversion to product. The reaction was chilled in a new ice bath at 0° C. To prevent hydrolysis of ester during quench, 3 mL of MeOH was slowly added to the reaction mixture and H₂ gas evolved. Then, sat. NH₄Cl was added slowly. The mixture was transferred to a separatory funnel and extracted 3× with EtOAc. The organic phases were combined, dried and concentrated in vacuo. The residue was purified via chromatography (EA:Hex gradient) to yield the desired product (475 mg, 42.8% yield) as a clear oil. LCMS: C₁₄H₂₅NO₅ requires: 287.1, found: m/z=288.2 [M+H]⁺

### Step 2: Synthesis of methyl 4-ethoxypiperidine-4-carboxylate hydrochloride

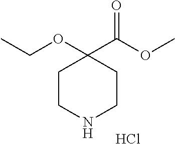

1-tert-butyl 4-methyl 4-ethoxypiperidine-1,4-dicarboxylate (475 mg, 1.65 mmol) was dissolved in 4M HCl in dioxane (4.00 mL) and stirred at RT. After 1 hr, the reaction was checked by LCMS and solid has precipitated out. Once complete, the reaction is concentrated in vacuo to give a white powder. The resulting solid is dried further on a high vacuum system to yield the product (350 mg, 94.6% yield) as a while solid. LCMS: C₉H₁₇NO₃ requires: 223.1, found: m/z=224.1 [M+H]⁺

### Step 3: Synthesis of methyl 1-(6-chloropyridazin-4-yl)-4-ethoxypiperidine-4-carboxylate

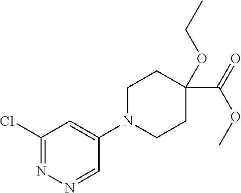 [SMILES: CCOC1(C(=O)OC)CCN(c2cnnc(Cl)c2)CC1]

Methyl 4-ethoxypiperidine-4-carboxylate hydrochloride (342 mg, 1.52 mmol) and 3,5-dichloropyridazine (227 mg, 1.52 mmol) were dissolved in DMSO (3.0 mL) under argon atmosphere. N,N-diisopropylethylamine (DIPEA) (987 mg, 7.64 mmol) was then added and the resulting solution was stirred at 100° C. for 12 hours. The reaction was monitored via LCMS. Once complete, the reaction was concentrated in vacuo to remove excess DIPEA and then quenched with H₂O and extracted with EtOAc 3 times. The organic phases were combined, dried and concentrated in vacuo. The residue was purified via chromatography (EA:hexane, gradient) to produce a red oil. Diethyl ether (10 mL) was added to the red oil and a pink solid was precipitated out. The solid was collected and further dried to yield the product (423 mg, 92.3% yield) as a pink solid. LCMS: C₁₃H₁₈ClN₃O₃ requires: 299.0, found: m/z=300.1 [M+H]⁺

### Step 4: Synthesis of methyl 4-ethoxy-1-(6-(2-hydroxyphenyl)pyridazin-4-yl)piperidine-4-carboxylate

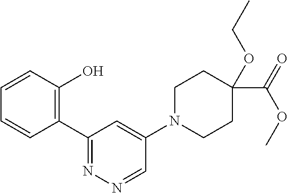 [SMILES: CCOC1(C(=O)OC)CCN(c2cnnc(-c3ccccc3O)c2)CC1]

A flask was charged with methyl 1-(6-chloropyridazin-4-yl)-4-ethoxypiperidine-4-carboxylate (423 mg, 1.41 mmol), potassium carbonate (585 mg, 4.23 mmol), and 2-hydroxyphenylboronic acid (291 mg, 2.11 mmol). 1,4-dioxane (5.60 mL) and water (1.20 mL) were sequentially added to the flask. The resulting solution was degassed with an argon balloon for 5 mins and then tetrakis(triphenylphosphine)palladium(O) (163 mg, 0.141 mmol) was added in one portion. The flask was sealed and the resulting mixture was stirred at 100° C. for 18 h. Upon completion, the flask was diluted in EtOAc and sat. NH₄Cl was added. The mixture was extracted 3× with EtOAc and the organic phases were concentrated in vacuo. The residue was purified via chromatography (EA:hexane, gradient) to give the desired product (440 mg, 87% yield) as an orange oil. LCMS: C₂₀H₂₅N₃O₅ requires: 357.1, found: m/z=358.2 [M+H]⁺

### Step 5: Synthesis of the Title Compound

Methyl 4-ethoxy-1-[6-(2-hydroxyphenyl)pyridazin-4-yl]piperidine-4-carboxylate (169 mg, 0.472 mmol) was dissolved in THF (2.50 mL) and H₂O (2.50 mL). Lithium hydroxide monohydrate (39.6 mg, 0.945 mmol) was added in one portion. The reaction was stirred at RT until complete by LCMS. Once complete, the reaction mixture is concentrated in vacuo and lyophilized to afford the desired lithium salt (190 mg, 113% yield) as a yellow powder. LCMS: C₁₈H₁₉N₃O₄ requires: 342.1, found: m/z=343.1 [M+H]⁺

### Intermediate 5

### RAC-(R)-3-METHOXY-1-(6-(2-(METHOXYMETHOXY)PHENYL)PYRIDAZIN-4-YL)PYRROLIDINE-3-CARBOXYLIC ACID

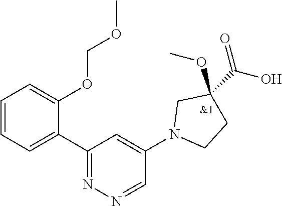 [SMILES: COCOc1ccccc1-c1cc(N2CC[C@@](OC)(C(=O)O)C2)cnn1]

### Step 1: Synthesis of rac-methyl (R)-1-(6-chloropyridazin-4-yl)-3-methoxypyrrolidine-3-carboxylate

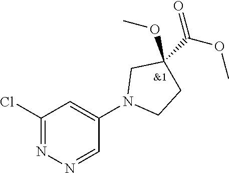 [SMILES: COC(=O)[C@]1(OC)CCN(c2cnnc(Cl)c2)C1]

This compound was prepared as described for the synthesis of Intermediate 1 Step 3, using rac-methyl (R)-3-methoxypyrrolidine-3-carboxylate to afford the desired compound (906.1 mgs, 65% yield) as a yellow oil. LCMS C₁₁H₁₄ClN₃O₃ requires 271.2 found m/z=272.2 [M+H]⁺.

### Step 2: Synthesis of rac-methyl (R)-3-methoxy-1-(6-(2-(methoxymethoxy)phenyl)pyridazin-4-yl)pyrrolidine-3-carboxylate

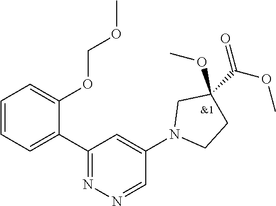 [SMILES: COCOc1ccccc1-c1cc(N2CC[C@@](OC)(C(=O)OC)C2)cnn1]

This compound was prepared as described for the synthesis of Intermediate 1 Step 4, using rac-methyl (R)-1-(6-chloropyridazin-4-yl)-3-methoxypyrrolidine-3-carboxylate to afford the desired compound (813.9 mg, 65% yield) as a yellow oil. LCMS C₁₉H₂₃N₃O₅ requires 373.2, found m/z=374.2 [M+H]⁺.

### Step 3: Synthesis of Title Intermediate

The title intermediate compound was prepared as described for Intermediate 1 Step 5, using rac-methyl (R)-3-methoxy-1-(6-(2-(methoxymethoxy)phenyl)pyridazin-4-yl)pyrrolidine-3-carboxylate. An additional acidification step to pH 7 with 3M HCl was performed followed by evaporation to dryness to isolate the title compound as the free acid in quantitative yield as a yellow oil. LCMS C₁₈H₂₁N₃O₅ requires 359.2, found m/z=360.2 [M+H]⁺.

### Intermediate 6

### 2-(1-(2-(2,6-DIOXOPIPERIDIN-3-YL)-1,3-DIOXOISOINDOLIN-5-YL)PIPERIDIN-4-YL)ACETALDEHYDE

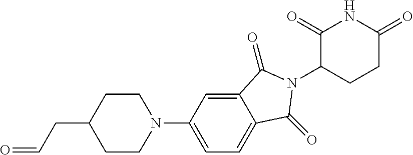 [SMILES: O=CCC1CCN(c2ccc3c(c2)C(=O)N(C2CCC(=O)NC2=O)C3=O)CC1]

### Step 1: Synthesis of 2-(2,6-dioxopiperidin-3-yl)-5-fluoroisoindoline-1,3-dione

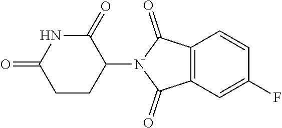 [SMILES: O=C1CCC(N2C(=O)c3ccc(F)cc3C2=O)C(=O)N1]

A mixture of 5-fluoro-1,3-dihydro-2-benzofuran-1,3-dione (5.00 g, 30.10 mmol), 3-aminopiperidine-2,6-dione hydrochloride (6.90 g, 42.14 mmol) and NaOAc (4.20 g, 51.17 mmol) in HOAc (50 mL) was stirred at 120° C. for 5 h before concentrated under vacuum. The residue was washed with water and the solid was collected by filtration. The crude product was washed with water twice and ethyl acetate twice and dried under oven to afford 2-(2,6-dioxopiperidin-3-yl)-5-fluoroisoindoline-1,3-dione (7.70 g, 92%) as a light brown solid. ¹H NMR (300 MHz, DMSO-d₆) δ 11.16 (s, 1H), 8.03-8.00 (m, 1H), 7.87-7.85 (m, 1H), 7.75-7.70 (m, 1H), 5.19-5.15 (m, 1H), 2.94-2.86 (m, 1H), 2.63-2.48 (m, 2H), 2.12-2.06 (m, 1H). F NMR (300 MHz, DMSO-d₆) δ −102.078.

### Step 2: Synthesis of 2-(2,6-dioxopiperidin-3-yl)-5-(4-(2-hydroxyethyl)piperidin-1-yl)isoindoline-1,3-dione

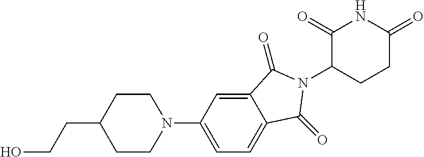 [SMILES: O=C1CCC(N2C(=O)c3ccc(N4CCC(CCO)CC4)cc3C2=O)C(=O)N1]

To a solution of 2-(2,6-dioxopiperidin-3-yl)-5-fluoro-2,3-dihydro-1H-isoindole-1,3-dione (1.00 g, 3.62 mmol) in N-Methyl pyrrolidone (10.00 mL) were added 2-(piperidin-4-yl)ethan-1-ol (3.60 mmol) and DIEA (1.40 g, 10.83 mmol). The resulting solution was stirred at 80° C. for 16 h. The reaction mixture was cooled down to room temperature and purified by reverse phase flash chromatography to afford 2-(2,6-dioxopiperidin-3-yl)-5-(4-(2-hydroxyethyl)piperidin-1-yl)isoindoline-1,3-dione (822.80 mg, 59%) as a yellow solid. ¹H NMR (300 MHz, DMSO-d₆) δ 11.09 (s, 1H), 7.65 (d, J 8.4 Hz, 1H), 7.30 (d, J 2.4 Hz, 1H), 7.23 (dd, J 8.4, 2.4 Hz, 1H), 5.07 (dd, J 12.6, 5.4 Hz, 1H), 4.40 (t, J 5.1 Hz, 1H), 4.04 (d, J 13.2 Hz, 2H), 3.64-3.40 (m, 2H), 3.09-2.79 (m, 3H), 2.70-2.51 (m, 2H), 2.07-1.94 (m, 1H), 1.77-1.66 (m, 3H), 1.41-1.34 (m, 2H), 1.24-1.12 (m, 2H). MS (ESI) C₂₀H₂₃N₃O₅ [M+H]⁺ requires: 386.2; found 386.1.

### Step 3: Synthesis of Title Compound

To a mixture of the 2-(2,6-dioxopiperidin-3-yl)-5-(4-(2-hydroxyethyl)piperidin-1-yl)isoindoline-1,3-dione (1.06 mmol) in CH₂Cl₂ (10 mL) was added Dess-Martin periodinane (2.12 mmol). The mixture was allowed to stir at room temperature for 1 h. The mixture was purified by column chromatography to afford the title compound as a clear oil. LCMS C₂₀H₂₁N₃O₅ requires: 383, found: m/z=384 [M+H]⁺.

### Intermediate 7

### 1-(4-(2,4-DIOXOTETRAHYDROPYRIMIDIN-1(2H)-YL)PHENYL)PIPERIDINE-4-CARBALDEHYDE

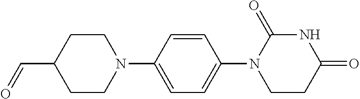 [SMILES: O=CC1CCN(c2ccc(N3CCC(=O)NC3=O)cc2)CC1]

### Step 1: Synthesis of 1-(4-bromophenyl)-3-(4-methoxybenzyl)dihydropyrimidine-2,4(1H,3H)-dione

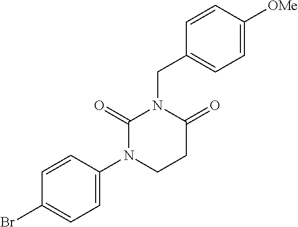 [SMILES: COc1ccc(CN2C(=O)CCN(c3ccc(Br)cc3)C2=O)cc1]

To a 40 mL vial was added 1-(4-bromophenyl)-1,3-diazinane-2,4-dione (6.40 g, 24.00 mmol), cesium carbonate (9.30 g, 28.50 mmol), and DMF (60.00 mL). To the reaction mixture was added 4-methoxybenzyl chloride (4.17 mL, 4.84 g, 30.90 mmol). The reaction mixture was stirred overnight, then quenched with water. The product was extracted with DCM (3×), dried over Na₂SO₄, then concentrated. The resulting residue was purified by FC (330 g silica, 0-50% EtOAc/DCM). The resulting purified residue was concentrated under reduced pressure into an amorphous solid and triturated with hexanes to yield 1-(4-bromophenyl)-3-(4-methoxybenzyl)dihydropyrimidine-2,4(1H,3H)-dione as a white solid (9.26 g, quantitative yield). LCMS: C₁₈H₁₇BrN₂O₃ requires: 388.0, found: m/z=389.2 [M+H]⁺.

### Step 2: Synthesis of 1-(4-(4-(1,3-dioxolan-2-yl)piperidin-1-yl)phenyl)-3-(4-methoxybenzyl)dihydropyrimidine-2,4(1H,3H)-dione

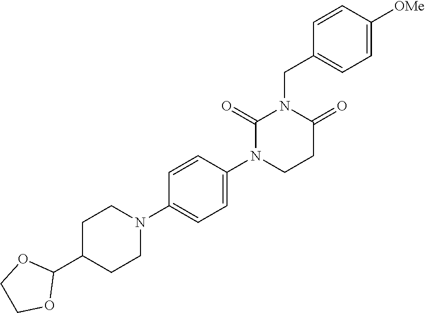 [SMILES: COc1ccc(CN2C(=O)CCN(c3ccc(N4CCC(C5OCCO5)CC4)cc3)C2=O)cc1]

To a 2 dram vial was added 1-(4-bromophenyl)-3-[(4-methoxyphenyl)methyl]-1,3-diazinane-2,4-dione (550.00 mg, 1.41 mmol), 4-(1,3-dioxolan-2-yl)piperidine (666.00 mg, 4.24 mmol), di-tert-butyl({2′,4′,6′-triisopropyl-[1,1′-biphenyl]-2-yl})phosphane (240 mg, 0.57 mmol), palladium (II) acetate (63.00 mg, 0.28 mmol), cesium carbonate (1.38 g, 4.24 mmol), and dioxane (5.00 mL). The reaction mixture was sparged with N₂ for 10 min, then stirred at 95° C. overnight. The crude reaction mixture was diluted with DCM and water, then filtered through celite. The product was extracted with DCM (3×), dried over MgSO₄, then concentrated. The resulting residue was purified by FC (40 g silica, 0-100% EtOAc/hex) to yield 1-(4-(4-(1,3-dioxolan-2-yl)piperidin-1-yl)phenyl)-3-(4-methoxybenzyl)dihydropyrimidine-2,4(1H,3H)-dione as a white solid (395.00 mg, 60%). LCMS: C₂₆H₃₁N₃O₅ requires: 465.2, found: m/z=466.4 [M+H]⁺.

### Step 3: Synthesis of Title Compound

To a 20 mL vial was added 1-{4-[4-(1,3-dioxolan-2-yl)piperidin-1-yl]phenyl}-3-[(4-methoxyphenyl)methyl]-1,3-diazinane-2,4-dione (395.00 mg, 0.85 mmol) and TFA (5.00 mL). To the reaction mixture was added trifluoromethanesulfonic acid (374.00 uL, 636.00 mg, 4.24 mmol). Full conversion after 10 min. The reaction mixture was diluted with minimal water, then purified by RP—FC (415 g C18 silica, 0-50% MeCN/water+0.1% TFA) to yield 1-(4-(2,4-dioxotetrahydropyrimidin-1(2H)-yl)phenyl)piperidine-4-carbaldehyde as a white solid (40.00 mg, 16%). LCMS: C₁₆H₂₁N₃O₃ requires: 301.1, found: m/z=302.2 [M+H]⁺.

### Intermediate 8

### 2-(1-(5-(2,6-DIOXOPIPERIDIN-3-YL)PYRIDIN-2-YL)PIPERIDIN-4-YL)ACETALDEHYDE

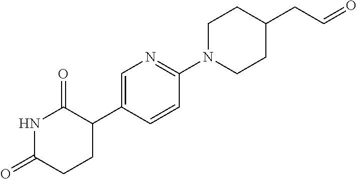 [SMILES: O=CCC1CCN(c2ccc(C3CCC(=O)NC3=O)cn2)CC1]

### Step 1: Synthesis of 2-(1-(5-bromopyridin-2-yl)piperidin-4-yl)ethan-1-ol

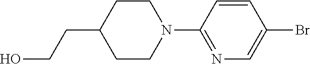 [SMILES: OCCC1CCN(c2ccc(Br)cn2)CC1]

To a solution of 5-bromo-2-fluoropyridine (25.00 g, 142.00 mmol, 14.60 mL, 1.00 eq) and 2-(piperidin-4-yl)ethan-1-ol (27.50 g, 213.00 mmol, 1.50 eq) in ACN (250 mL) was added K₂CO₃ (58.90 g, 426.00 mmol, 3.00 eq). The mixture was stirred at 80° C. for 12 hrs. The mixture was poured into water and extracted with ethyl acetate, the combined organic layer was washed with brine, dried over Na₂SO₄, filter and concentrated under vacuum to get the crude product. The crude product was purified by chromatography on silica gel with petroleum ether: ethyl acetate=100:1 to 1:1. (Product: petroleum ether:ethyl acetate=0:1, R_{f}=0.40). 2-(1-(5-bromopyridin-2-yl)piperidin-4-yl)ethan-1-ol (44.00 g, 150.00 mmol, 75.8% yield, 97.8% purity) was obtained as a white solid. LCMS C₁₂H₁₇BrN₂O requires: 284.0, found: 285.2 [M+H]⁺.

### Step 2: Synthesis of 2-(1-(2′,6′-bis(benzyloxy)-[3,3′-bipyridin]-6-yl)piperidin-4-yl)ethan-1-ol

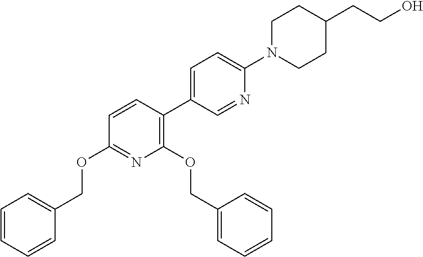 [SMILES: OCCC1CCN(c2ccc(-c3ccc(OCc4ccccc4)nc3OCc3ccccc3)cn2)CC1]

To a solution of 2-(1-(5-bromopyridin-2-yl)piperidin-4-yl)ethan-1-ol (22.00 g, 75.40 mmol, 1.00 eq) and 2,6-bis(benzyloxy)-3-(4,4,5,5-tetramethyl-1,3,2-dioxaborolan-2-yl)pyridine (33.00 g, 79.20 mmol, 1.05 eq) in dioxane (220 mL) and H₂O (44.0 mL) was added K₂CO₃ (31.20 g, 226.00 mmol, 3.00 eq), then Pd(dppf)Cl₂ (2.76 g, 3.77 mmol, 0.05 eq) was added to the mixture under N₂. The reaction mixture was stirred at 100° C. for 12 hrs. The mixture was poured into water and extracted with ethyl acetate, the combined organic layer was washed with brine, dried over Na₂SO₄, filter and concentrated under vacuum to get the crude product. The residue was purified by column chromatography (SiO₂, petroleum ether: Ethyl acetate=1:10 to 0:1, petroleum ether: ethyl acetate=1:1, R_{f}=0.30). 2-(1-(2′,6′-bis(benzyloxy)-[3,3′-bipyridin]-6-yl)piperidin-4-yl)ethan-1-ol (60.00 g, 113.00 mmol, 74.9% yield, 93.4% purity) was obtained as a yellow solid. LCMS C₃₁H₃₃N₃O₃ requires: 495.2, found: 496.3 [M+H]⁺.

### Step 3: Synthesis of 3-(6-(4-(2-hydroxyethyl)piperidin-1-yl)pyridin-3-yl)piperidine-2,6-dione

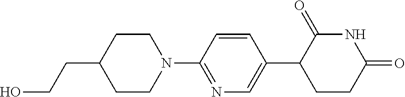 [SMILES: O=C1CCC(c2ccc(N3CCC(CCO)CC3)nc2)C(=O)N1]

To a solution of 2-(1-(2′,6′-bis(benzyloxy)-[3,3′-bipyridin]-6-yl)piperidin-4-yl)ethan-1-ol (30.00 g, 56.50 mmol, 1.00 eq) in THF (150.00 mL) and EtOH (150.00 mL) was added Pd/C (9.00 g, 10% purity) and Pd(OH)₂ (9.00 g, 20% purity) under N₂. The suspension was degassed and purged with H₂ for 3 times. The mixture was stirred under H₂ (50 Psi) at 25° C. for 12 hrs. The mixture was filtered to get the filtrate. The filtrate was concentrated. The crude product was triturated with ethyl acetate (3 V, 90.00 mL) at 25° C. for 30 mins. 3-(6-(4-(2-hydroxyethyl)piperidin-1-yl)pyridin-3-yl)piperidine-2,6-dione (11.00 g, 32.60 mmol, 57.6% yield, 94.1% purity) was obtained as a yellow solid. LCMS C₁₇H₂₃N₃O₃ requires: 317.2, found: 318.2 [M+H]⁺.

### Step 4: Synthesis of Title Compound

To a solution of 3-(6-(4-(2-hydroxyethyl)piperidin-1-yl)pyridin-3-yl)piperidine-2,6-dione (7.00 g, 20.70 mmol, 1.00 eq), DIEA (8.05 g, 62.20 mmol, 10.80 mL, 3.00 eq) and DMSO (3.24 g, 41.50 mmol, 3.24 mL, 2.00 eq) in DCM (130 mL) was added slowly SO₃·Py (6.61 g, 41.50 mmol, 2.00 eq) at 0° C. The reaction was stirred at 0~5° C. for 1 hr. The reaction mixture was concentrated under the vacuum. The residue was purified by column chromatography (SiO₂, petroleum ether:Ethyl acetate to Dichloromethane:Methanol=10:1 to 10:1) (Ethyl acetate:Methanol=5:1, R_{f}=0.60). 2-(1-(5-(2,6-dioxopiperidin-3-yl)pyridin-2-yl)piperidin-4-yl)acetaldehyde (8.78 g, 26.60 mmol, 64.1% yield, 95.7% purity) was obtained as a pink solid. LCMS C₁₇H₂₁N₃O₃ requires: 315.2, found: m/z=316.2 [M+H]⁺.

### Intermediate 9

### 1-(5-(2,6-DIOXOPIPERIDIN-3-YL)PYRIMIDIN-2-YL)PIPERIDINE-4-CARBALDEHYDE

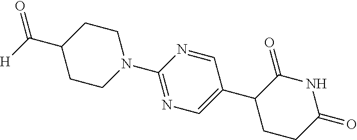 [SMILES: O=CC1CCN(c2ncc(C3CCC(=O)NC3=O)cn2)CC1]

### Step 1: Synthesis of (1-(5-chloropyrimidin-2-yl)piperidin-4-yl)methanol

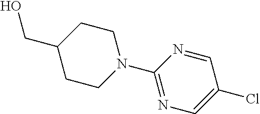 [SMILES: OCC1CCN(c2ncc(Cl)cn2)CC1]

To a solution piperidin-4-ylmethanol (18.00 g, 156.00 mmol, 1.00 eq) and 2,5-dichloropyrimidine (23.20 g, 156.00 mmol, 1.00 eq) in ACN (180 mL) was added TEA (63.2 g, 625.00 mmol, 87.00 mL, 4.00 eq) at 20° C. The reaction was stirred at 90° C. for 12 hrs. The mixture was cooled to 20° C. and filtered. The filtrate was concentrated. The crude product was triturated with petroleum ether/Ethyl acetate=10/1 (200 mL) at 20° C. for 12 hrs. The crude product was triturated with H₂O (200 mL) at 20° C. for 30 mins. (1-(5-chloropyrimidin-2-yl)piperidin-4-yl)methanol (30.00 g, 131.00 mmol, 83.9% yield, 99.6% purity) was obtained as white solid. LCMS C₁₀H₁₄ClN₃O requires: 227.1, found: m/z=228.0 [M+H]⁺.

### Step 2: Synthesis of (1-(5-(2,6-bis(benzyloxy)pyridin-3-yl)pyrimidin-2-yl)piperidin-4-yl)methanol

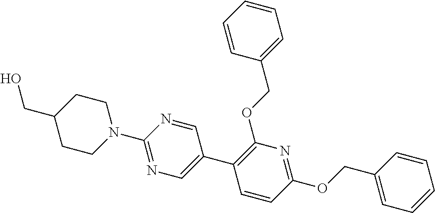 [SMILES: OCC1CCN(c2ncc(-c3ccc(OCc4ccccc4)nc3OCc3ccccc3)cn2)CC1]

To a solution of (1-(5-chloropyrimidin-2-yl)piperidin-4-yl)methanol (10.00 g, 43.70 mmol, 99.6% purity, 1.00 eq) and 2,6-bis(benzyloxy)-3-(4,4,5,5-tetramethyl-1,3,2-dioxaborolan-2-yl)pyridine (18.20 g, 43.70 mmol, 1.00 eq) in dioxane (100.00 mL) and H₂O (33.00 mL) was added K₂CO₃ (15.10 g, 109.00 mmol, 2.50 eq) and SPhos Pd G₂ (1.58 g, 2.19 mmol, 0.05 eq) at 20° C. under N₂. The reaction was stirred at 100° C. for 2 hrs under N₂. The reaction was cooled to 20° C. and concentrated. The residue was poured into water. The mixture was extracted with ethyl acetate. The combined organic layers were washed with brine, dried over Na₂SO₄, filtered and concentrated. The crude product was purified by reversed-phase HPLC (0.1% FA condition). The product was concentrated under vacuum. The residue was extracted with ethyl acetate. The combined layers were washed with brine, dried over Na₂SO₄, filtered and concentrated. The crude product was triturated with petroleum ether/Ethyl acetate (1/1, 150 mL) at 20° C. for 20 mins. (1-(5-(2,6-bis(benzyloxy)pyridin-3-yl)pyrimidin-2-yl)piperidin-4-yl)methanol (14.00 g, 28.30 mmol, 64.7% yield, 97.6% purity) was obtained as white solid. LCMS C₂₉H₃₀N₄O₃ requires: 482.2, found: m/z=483.2 [M+H]⁺.

### Step 3: Synthesis of 3-(2-(4-(hydroxymethyl)piperidin-1-yl)pyrimidin-5-yl)piperidine-2,6-dione

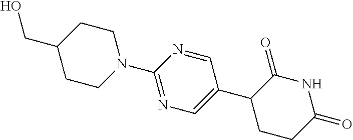 [SMILES: O=C1CCC(c2cnc(N3CCC(CO)CC3)nc2)C(=O)N1]

To a solution of (1-(5-(2,6-bis(benzyloxy)pyridin-3-yl)pyrimidin-2-yl)piperidin-4-yl)methanol (4.00 g, 8.09 mmol, 97.6% purity, 1.00 eq) in THF (110 mL) was added Pd/C (4.00 g, 10% purity) under N₂. The suspension was degassed under vacuum and purged with H₂ several times. The mixture was stirred under H₂ (15 psi) at 20° C. for 12 hrs. The mixture was filtered without diatomite and the combined filtrate was concentrated at N₂. 3-(2-(4-(hydroxymethyl)piperidin-1-yl)pyrimidin-5-yl)piperidine-2,6-dione (3.90 g, crude) was obtained as light yellow solid without purification. LCMS C₁₅H₂₀N₄O₃ requires: 304.1, found: m/z=305.2 [M+H]⁺.

### Step 4: Synthesis of Title Compound

To a solution of 3-(2-(4-(hydroxymethyl)piperidin-1-yl)pyrimidin-5-yl)piperidine-2,6-dione (1.30 g, 3.17 mmol, 74.2% purity, 1.00 eq) in DMSO (36.0 mL) was slowly added DMP (3.62 g, 8.54 mmol, 2.64 mL, 2.00 eq) at 20° C. The reaction was stirred at 20° C. for 1 hr. The mixture was poured into ice-water (200 mL) and adjusted with saturated aqueous Na₂CO₃ until pH=10 at 0° C. and the aqueous layer was extracted with Ethyl acetate (1200 mL*3). The combined organic layers were washed with Na₂S₂O₃ solution (500 mL*2) and brine (1000 mL*2), dried over Na₂SO₄, concentrated under vacuum. The crude product was triturated with ethyl acetate (20.0 mL) at 20° C. for 12 hrs. 1-(5-(2,6-dioxopiperidin-3-yl)pyrimidin-2-yl)piperidine-4-carbaldehyde (0.95 g, 3.12 mmol, 24.3% yield, 99.4% purity) was obtained as white solid. LCMS C₁₅H₁₈N₄O₃ requires: 302.1, found: m/z=301.1 [M−H]⁺. ¹H NMR: 400 MHz, DMSO-d₆ δ 10.86 (s, 1H), 9.62 (s, 1H), 8.23 (s, 2H), 4.47-4.39 (m, 2H), 3.77-3.70 (m, 1H), 3.19-3.10 (m, 2H), 2.56 (t, J=3.6 Hz, 3H), 2.29-2.16 (m, 1H), 2.02-1.94 (m, 1H), 1.93-1.85 (m, 2H), 1.49-1.38 (m, 2H).

### Intermediate 10

### 1-(3-(2,6-DIOXOPIPERIDIN-3-YL)-4-FLUOROPHENYL)PIPERIDINE-4-CARBALDEHYDE

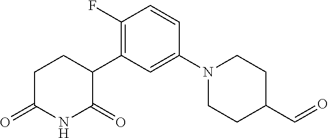 [SMILES: O=CC1CCN(c2ccc(F)c(C3CCC(=O)NC3=O)c2)CC1]

### Step 1: Synthesis of 1-(3-bromo-4-fluorophenyl)-4-(dimethoxymethyl)piperidine

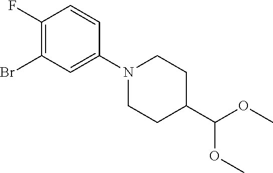 [SMILES: COC(OC)C1CCN(c2ccc(F)c(Br)c2)CC1]

To a solution of 2-bromo-1-fluoro-4-iodobenzene (40.00 g, 133.00 mmol, 1.00 eq), 4-(dimethoxymethyl)piperidine (23.30 g, 146.00 mmol, 1.10 eq), L-PROLINE (6.12 g, 53.20 mmol, 0.40 eq) and Cs₂CO₃ (86.60 g, 266.00 mmol, 2.00 eq) in DMSO (280 mL) was added CuI (5.06 g, 26.60 mmol, 0.200 eq), then stirred at 80° C. for 12 hrs. The mixture was diluted with H₂O, extracted with MTBE, the combined organic layer was washed with 3% NH₃·H₂O and brine, dried over Na₂SO₄, filtered and concentrated under reduced pressure to afford 1-(3-bromo-4-fluorophenyl)-4-(dimethoxymethyl)piperidine (25.50 g, 68.20 mmol, 51.3% yield, 88.9% purity) as an yellow oil, which was used for next step without further purification. LCMS C₁₄H₁₉BrFNO₂ requires: 333.0, found: m/z=334 [M+H]⁺.

### Step 2: Synthesis of 2,6-bis(benzyloxy)-3-(5-(4-(dimethoxymethyl)piperidin-1-yl)-2-fluorophenyl)pyridine

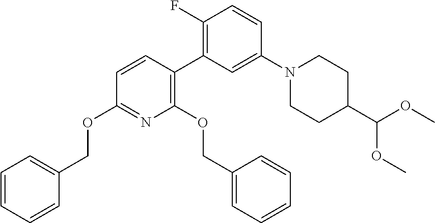 [SMILES: COC(OC)C1CCN(c2ccc(F)c(-c3ccc(OCc4ccccc4)nc3OCc3ccccc3)c2)CC1]

To a solution of 1-(3-bromo-4-fluorophenyl)-4-(dimethoxymethyl)piperdine (25.50 g, 68.20 mmol, 1.00 eq) in dioxane (500.00 mL) and H₂O (100.00 mL) was added 2,6-bis(benzyloxy)-3-(4,4,5,5-tetramethyl-1,3,2-dioxaborolan-2-yl)pyridine (40.30 g, 88.70 mmol, 1.30 eq), Cs₂CO₃ (55.60 g, 171.00 mmol, 2.50 eq) and Pd(dppf)Cl₂·CH₂Cl₂ (2.79 g, 3.41 mmol, 0.05 eq) at 25° C. under N₂. The reaction mixture was stirred at 100° C. for 12 hrs. The mixture was poured into H₂O, extracted with EtOAc, the combined organic layer was washed with brine, dried over Na₂SO₄, filtered and concentrated under reduced pressure. The crude was purified by MPLC (SiO₂, petroleum ether: Ethyl acetate=1:0 to 10:1, petroleum ether: Ethyl acetate=5:1, R_{f}=0.4), concentrated to afford 2,6-bis(benzyloxy)-3-(5-(4-(dimethoxymethyl)piperidin-1-yl)-2-fluorophenyl)pyridine (36.00 g, 64.80 mmol, 95.0% yield, 97.7% purity) as a yellow oil. LCMS C₃₃H₃₅FN₂O₄ requires: 542.2, found: m/z=543.4 [M+H]⁺.

### Step 3: Synthesis of 3-(5-(4-(dimethoxymethyl)piperidin-1-yl)-2-fluorophenyl)piperidine-2,6-dione

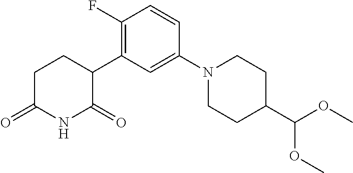 [SMILES: COC(OC)C1CCN(c2ccc(F)c(C3CCC(=O)NC3=O)c2)CC1]

A solution of 2,6-bis(benzyloxy)-3-(5-(4-(dimethoxymethyl)piperidin-1-yl)-2-fluorophenyl)pyridine (1.00 eq, 36.00 g) in dioxane (1.80 L) was stirred at 20° C. until becoming a clear solution. The fixed bed (named FLR1,volume 50 mL) was packed with granular catalyst 5% Pd/C (WXC1030), then heated to 40-55° C. The H₂ back pressure regulator was adjusted to 0.3 MPa. The above solution was pumped into the fixed bed {FLR1.5 min} at a flow rate of {2 mL/min}, and the flow rate of H₂ was 98 mL/min. The reaction mixture was collected after running 30 mins. After the reaction was completed, the fixed bed was washed by extra {dioxane,100 mL}. The solution was concentrated under reduced pressure to obtain the crude material, which was triturated with petroleum ether:Ethyl acetate=5:1 (100 mL) at 25° C. for 1 hr, filtered and the cake was dried under reduced pressure to afford 3-(5-(4-(dimethoxymethyl)piperidin-1-yl)-2-fluorophenyl)piperidine-2,6-dione (19.40 g, 49.30 mmol, 76.1% yield, 92.6% purity) as a white solid. LCMS C₁₉H₂₅FN₂O₄ requires: 364.2, found: m/z=365.3 [M+H]⁺.

### Step 4: Synthesis of Title Compound

To a solution of 3-(5-(4-(dimethoxymethyl)piperidin-1-yl)-2-fluorophenyl)piperidine-2,6-dione (10.00 g, 25.40 mmol, 1.00 eq) in THF (250.00 mL) was added HCl (2 M, 226.00 mL, 17.80 eq). After addition, the reaction solution was stirred at 70° C. for 1 hr. The reaction mixture was cooled to 25° C., then added saturated NaHCO₃ solution until pH reached 7, then extracted with EtOAc, the combined organic layer was washed with brine, dried over Na₂SO₄, filtered and concentrated to get the crude. The crude was triturated with MTBE (30.00 mL) at 25° C. for 0.5 hr, filtered and the cake was dried under reduced pressure. The residue was diluted with DCM until the mixture turned to clear and concentrated to afford 1-(3-(2,6-dioxopiperidin-3-yl)-4-fluorophenyl)piperidine-4-carbaldehyde (5.55 g, 17.10 mmol, 67.1% yield, 97.8% purity) as an off-white solid. LCMS C₁₇H₁₉FN₂O₃ requires: 318.1, found: m/z=337.1 [M+H₂O+H]⁺. H NMR: (400 MHz, CDCl₃) δ 9.70 (s, 1H), 8.42 (br s, 1H), 7.03-6.95 (m, 1H), 6.90-6.82 (m, 1H), 6.74 (dd, J=2.8, 6.0 Hz, 1H), 3.84 (dd, J=5.2, 11.2 Hz, 1H), 3.50-3.45 (m, 2H), 2.87-2.59 (m, 4H), 2.44-2.33 (m, 1H), 2.32-2.15 (m, 2H), 2.10-1.98 (m, 2H), 1.84-1.72 (m, 2H).

### Intermediate 11

### 2-(4-(4-(2,6-DIOXOPIPERIDIN-3-YL)PHENYL)-1-OXA-4,9-DIAZASPIRO[5.5]UNDECAN-9-YL)ACETIC ACID

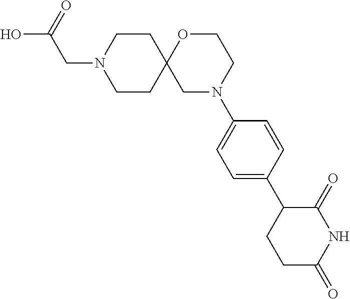 [SMILES: O=C(O)CN1CCC2(CC1)CN(c1ccc(C3CCC(=O)NC3=O)cc1)CCO2]

### Step 1: Synthesis of tert-butyl 4-(4-(2,6-bis(benzyloxy)pyridin-3-yl)phenyl)-1-oxa-4,9-diazaspiro[5.5]undecane-9-carboxylate

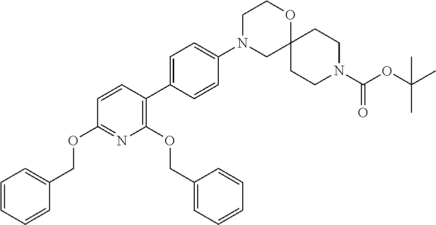 [SMILES: CC(C)(C)OC(=O)N1CCC2(CC1)CN(c1ccc(-c3ccc(OCc4ccccc4)nc3OCc3ccccc3)cc1)CCO2]

To a stirred solution of 2,6-bis(benzyloxy)-3-(4-bromophenyl)pyridine (5.00 g, 11.20 mmol) and tert-butyl 1-oxa-4,9-diazaspiro[5.5]undecane-9-carboxylate (2.87 g, 11.20 mmol) in 1,4 dioxane (50.00 mL) was added cesium carbonate (7.30 g, 22.40 mmol) at room temperature. The resulting mixture was purged with N₂ for 10 min. Then Pd₂(dba)₃ (0.51 g, 0.56 mmol) and RuPhos (0.52 g, 1.12 mmol) were added. The resulting mixture was purged with N₂ for 5 min and heated to 100° C. for 16 h. Upon completion of reaction, as confirmed by LCMS, the reaction mixture was cooled to RT and filtered through Celite pad and washed with ethyl acetate (300 mL). The filtrate was diluted with water and extracted with ethyl acetate. The combined organic layer was dried over sodium sulphate and concentrated under vacuum to afford the crude compound as brown liquid. The crude compound was purified by Biotage-Isolera (silica-gel: 230-400 mesh) using ethyl acetate/pet-ether (0-30%) as eluent to afford tert-butyl 4-(4-(2,6-bis(benzyloxy)pyridin-3-yl)phenyl)-1-oxa-4,9-diazaspiro[5.5]undecane-9-carboxylate (5.60 g, 77% yield) as pale yellow solid. LCMS C₃₈H₄₃N₃O₅ requires: 621.3, found: m/z=622.2 [M+H]⁺.

### Step 2: Synthesis of 4-(4-(2,6-bis(benzyloxy)pyridin-3-yl)phenyl)-1-oxa-4,9-diazaspiro[5.5]undecane

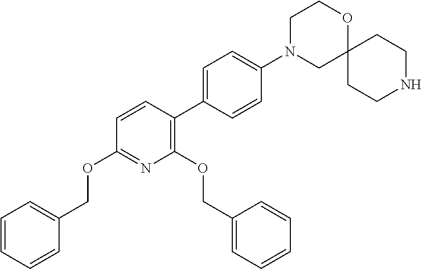 [SMILES: c1ccc(COc2ccc(-c3ccc(N4CCOC5(CCNCC5)C4)cc3)c(OCc3ccccc3)n2)cc1]

To a stirred solution of tert-butyl 4-(4-(2,6-bis(benzyloxy)pyridin-3-yl)phenyl)-1-oxa-4,9-diazaspiro[5.5]undecane-9-carboxylate (5.60 g, 9.01 mmol) in DCM (30.00 mL) was added 4M HCl in dioxane (11.26 mL, 45.00 mmol) at 0° C. The resulting reaction mixture was stirred at RT for 2 h. Upon completion of reaction, as confirmed by LCMS, the reaction mixture was concentrated under reduced pressure and washed with n-hexane and dried to afford 4-(4-(2,6-bis(benzyloxy)pyridin-3-yl)phenyl)-1-oxa-4,9-diazaspiro[5.5]undecane (4.50 g, 81% yield, HCl salt) as off-white solid. LCMS C₃₃H₃₅N₃O₃ requires: 521.3, found: m/z=522.4 [M+H]⁺.

### Step 3: Synthesis of tert-butyl 2-(4-(4-(2,6-bis(benzyloxy)pyridin-3-yl)phenyl)-1-oxa-4,9-diazaspiro[5.5]undecan-9-yl)acetate

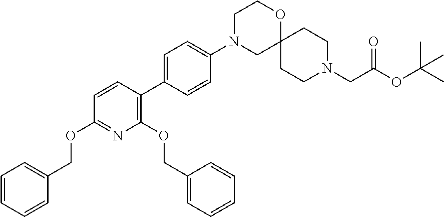 [SMILES: CC(C)(C)OC(=O)CN1CCC2(CC1)CN(c1ccc(-c3ccc(OCc4ccccc4)nc3OCc3ccccc3)cc1)CCO2]

To a solution of (4-(4-(2,6-bis(benzyloxy)pyridin-3-yl)phenyl)-1-oxa-4,9-diazaspiro[5.5]undecane (4.50 g, 7.33 mmol) in MeCN (50.00 mL) at RT were added triethylamine (5.11 ml, 36.70 mmol) and tert-butyl 2-chloroacetate (3.15 mL, 22.00 mmol). The reaction mixture was stirred at 60° C. for 16 h. Upon completion of reaction, as confirmed by LCMS, the reaction mixture was cooled to room temperature. Then water was added and extracted with EtOAc. The combined organic layer was washed with water, brine, dried over sodium sulphate and concentrated under reduced pressure to get the crude product. The crude product was purified by Biotage-Isolera (Silica gel: 230-400 mesh) using 30% of ethyl acetate/pet-ether as eluent to afford tert-butyl 2-(4-(4-(2,6-bis(benzyloxy)pyridin-3-yl)phenyl)-1-oxa-4,9-diazaspiro[5.5]undecan-9-yl)acetate (3.00 g, 61% yield) as off white solid. LCMS C₃₉H₄₅N₃O₅ requires: 635.3, found: m/z=637.4 [M+H]⁺.

### Step 4: Synthesis of tert-butyl 2-(4-(4-(2,6-dioxopiperidin-3-yl)phenyl)-1-oxa-4,9-diazaspiro[5.5]undecan-9-yl)acetate

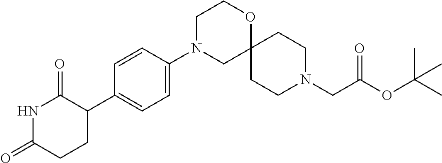 [SMILES: CC(C)(C)OC(=O)CN1CCC2(CC1)CN(c1ccc(C3CCC(=O)NC3=O)cc1)CCO2]

To a stirred solution of tert-butyl 2-(4-(4-(2,6-bis(benzyloxy)pyridin-3-yl)phenyl)-1-oxa-4,9-diazaspiro[5.5]undecan-9-yl)acetate (3.00 g, 4.72 mmol) in 1,4-dioxane (30 mL) was added Pd(OAc)₂ (0.40 g, 1.78 mmol) and 10% Pd/C (0.50 g, 4.70 mmol) under N₂ atmosphere at RT. The resulting mixture was stirred under H₂ pressure (1 atm) at RT for 24 h. Upon completion of the reaction, as confirmed by LCMS, the reaction mixture was filtered through Celite bed washed with ethyl acetate and concentrated under reduced pressure. The crude compound was washed with toluene, MTBE and concentrated under reduced pressure to afford tert-butyl2-(4-(4-(2,6-dioxopiperidin-3-yl)phenyl)-1-oxa-4,9-diazaspiro[5.5]undecan-9-yl)acetate (1.80 g, 83%) as off-white solid. LCMS C₂₅H₃₅N₃O₅ requires: 457.2, found: m/z=458.6 [M+H]⁺.

### Step 5: Synthesis of Title Compound

To a stirred solution tert-butyl2-(4-(4-(2,6-dioxopiperidin-3-yl)phenyl)-1-oxa-4,9-diazaspiro[5.5]undecan-9-yl)acetate (1.80 g, 3.93 mmol) in DCM (6.50 ml) at 0° C. was added HCl (4M soln. in dioxane, 9.83 ml, 39.30 mmol).The reaction mixture was stirred at RT for 4 h. Then n-hexane was added, stirred for 10 min and decanted and repeated twice. The resulting residue was concentrated, dried under reduced pressure The crude product was purified by prep-HPLC purification using: column: X-Select C18 19×250, method: HCl:MeCN, Flow rate:12 ml/min. collected fraction lyophilized to afford 2-(4-(4-(2,6-dioxopiperidin-3-yl)phenyl)-1-oxa-4,9-diazaspiro[5.5]undecan-9-yl)acetic acid (0.92 g, 64% yield, HCl salt) as an off-white solid LCMS C₂₁H₂₇N₃O₅ requires: 401.2, found: m/z=402.2 [M+H]⁺.

### Intermediate 12

### 3-(4-(PIPERAZIN-1-YL)PHENYL)PIPERIDINE-2,6-DIONE

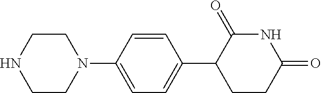 [SMILES: O=C1CCC(c2ccc(N3CCNCC3)cc2)C(=O)N1]

### Step 1: Synthesis of tert-butyl 4-(4-(2,6-bis(benzyloxy)pyridin-3-yl)phenyl)piperazine-1-carboxylate

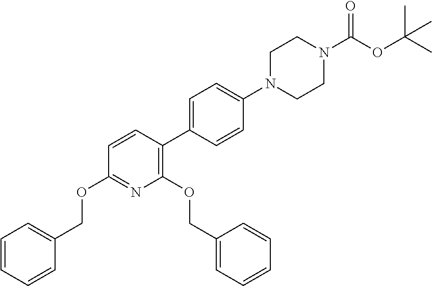 [SMILES: CC(C)(C)OC(=O)N1CCN(c2ccc(-c3ccc(OCc4ccccc4)nc3OCc3ccccc3)cc2)CC1]

2,6-bis(benzyloxy)-3-(4-bromophenyl)pyridine (79.20 g, 177.44 mmol, 1.0 eq), tert-Butyl 1-piperazinecarboxylate (39.66 g, 212.93 mmol, 1.20 eq), Cs₂CO₃ (289.07 g, 887.20 mmol, 5.0 eq), Pd₂(dba)₃ (12.19 g, 13.31 mmol, 0.075 eq), Ruphos (12.43 g, 26.62 mmol, 0.15 eq) were added successively into 1,4-dioxane (800 mL). The mixture was heated to 100° C. under argon and stirred for 16 h. The mixture was filtered through a Celite pad, and the filtrate was concentrated to give the crude product. The reaction mixture was diluted with water and extracted with EA. The organic layer was washed with saturated brine. The organic layer was dried over Na₂SO₄. The mixture was concentrated to afford the crude product. The crude product was purified by column chromatography on silica gel eluted with (PE/EA 8:1) to give a yellow solid. The yellow solid was recrystallized from PE/EA (8:1) and filtered to afford tert-butyl 4-(4-(2,6-bis(benzyloxy)pyridin-3-yl)phenyl)piperazine-1-carboxylate (48.10 g, 60.7%) a white solid.

### Step 2: Synthesis of tert-butyl 4-(4-(2,6-dioxopiperidin-3-yl)phenyl)piperazine-1-carboxylate

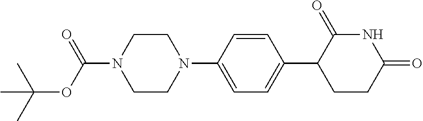 [SMILES: CC(C)(C)OC(=O)N1CCN(c2ccc(C3CCC(=O)NC3=O)cc2)CC1]

A mixture of tert-butyl 4-(4-(2,6-bis(benzyloxy)pyridin-3-yl)phenyl)piperazine-1-carboxylate (45.00 g, 81.60 mmol, 1.0 eq), Pd/C (9.00 g, 20% wt/wt) and Pd/(OAC)₂ (7.20 g) in 1,4-dioxane (450 mL) was hydrogenated under 50 Psi of hydrogen pressure overnight at room temperature. The mixture was filtered and concentrated to afford tert-butyl 4-(4-(2,6-dioxopiperidin-3-yl)phenyl)piperazine-1-carboxylate (27.90 g, 91.5%) as a grey solid. LCMS C₂₀H₂₇N₃O₄ requires: 373.2, found: m/z=374.9 [M+H]⁺.

### Step 3: Synthesis of Title Compound

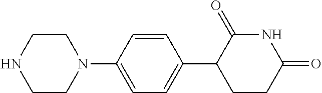 [SMILES: O=C1CCC(c2ccc(N3CCNCC3)cc2)C(=O)N1]

To a solution of tert-butyl 4-(4-(2,6-dioxopiperidin-3-yl)phenyl)piperazine-1-carboxylate (27.90 g, 74.70 mmol, 1.00 eq) in DCM (300 mL) was added 4M HCl in dioxane (261.50 mL, 1045.80 mmol, 14.0 eq). Then the reaction mixture was stirred for 2 h at room temperature. After that the mixture was filtered and the filter cake was triturated with PE/EA=10:1 (200 mL) and filtered to afford 3-(4-(piperazin-1-yl)phenyl)piperidine-2,6-dione (21.00 g, 91.3%) as an off-white solid. LCMS C₁₅H₁₉N₃O₂ requires: 273.1, found: m/z=274.0 [M+H]⁺.

### Intermediate 13

### 1-(6-(2-(METHOXYMETHOXY)PHENYL)PYRIDAZIN-4-YL)-4-(METHOXYMETHYL)PIPERIDINE-4-CARBOXYLIC ACID

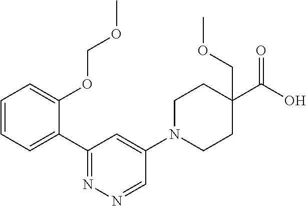 [SMILES: COCOc1ccccc1-c1cc(N2CCC(COC)(C(=O)O)CC2)cnn1]

### Step 1: Synthesis of ethyl 1-(6-chloropyridazin-4-yl)-4-(methoxymethyl)piperidine-4-carboxylate

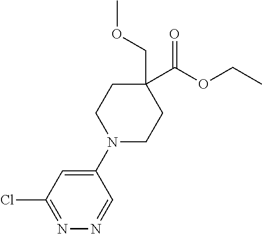 [SMILES: CCOC(=O)C1(COC)CCN(c2cnnc(Cl)c2)CC1]

This compound was prepared as described for the synthesis of Intermediate 1 Step 3, using ethyl 4-(methoxymethyl)piperidine-4-carboxylate (1.00 g, 4.97 mmol), affording the desired product (1.16 g, 75% yield) as a yellow oil. LCMS C₁₄H₂₀ClN₃O₃ requires 313.2, found m/z=314.2 [M+H]⁺.

### Step 2: Synthesis of ethyl 1-(6-(2-(methoxymethoxy)phenyl)pyridazin-4-yl)-4-(methoxymethyl)piperidine-4-carboxylate

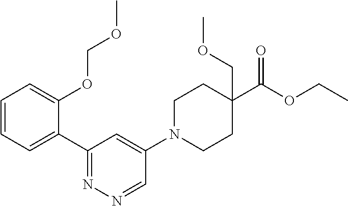 [SMILES: CCOC(=O)C1(COC)CCN(c2cnnc(-c3ccccc3OCOC)c2)CC1]

This compound was prepared as described or the synthesis of Intermediate 1 Step 4, using ethyl 1-(6-chloropyridazin-4-yl)-4-(methoxymethyl)piperidine-4-carboxylate (1.16 g, 3.70 mmol), affording the desired product (1.55 g, 96% yield) as a yellow oil. LCMS C₂₂H₂₉N₃O₅ requires 415.2, found m/z=416.2 [M+H]⁺.

### Step 3: Synthesis of Title Intermediate Compound

The title intermediate compound was prepared as described for the synthesis of Intermediate 1 Step 5, using ethyl 1-(6-(2-(methoxymethoxy)phenyl)pyridazin-4-yl)-4-(methoxymethyl)piperidine-4-carboxylate (573.6 mg, 1.38 mmol). An additional acidification step to pH 7 with 3M HCl was performed followed by evaporation to dryness to isolate the title compound as the free acid which was taken forward without any further purification. LCMS C₂₀H₂₅N₃O₅ requires 387.2, found [M+H]⁺.

### Intermediate 14

### (1R,4R)-4-(3-((RS)-2,6-DIOXOPIPERIDIN-3-YL)PHENOXY)CYCLOHEXANE-1-CARBALDEHYDE

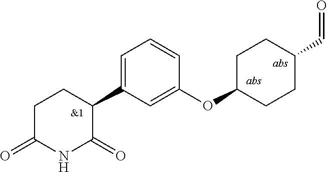 [SMILES: O=C[C@H]1CC[C@H](Oc2cccc([C@@H]3CCC(=O)NC3=O)c2)CC1]

### Step 1: Synthesis of 3-(2,6-bis(benzyloxy)pyridin-3-yl)phenol

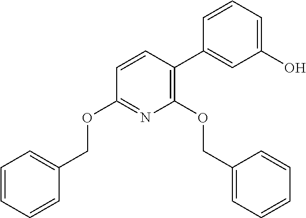 [SMILES: Oc1cccc(-c2ccc(OCc3ccccc3)nc2OCc2ccccc2)c1]

To a stirred solution of 3-hydroxyphenylboronic acid (15.00 g, 108.75 mmol, 1.00 equiv) and 2,6-bis(benzyloxy)-3-bromopyridine (52.34 g, 141.38 mmol, 1.30 equiv) in dioxane (50.00 mL)/H₂O (5.00 mL) were added K₂CO₃ (45.09 g, 326.25 mmol, 3.00 equiv) and Pd(dppf)Cl₂CH₂Cl₂ (17.72 g, 21.75 mmol, 0.20 equiv). The resulting mixture was stirred at 90° C. for 2 h under nitrogen atmosphere. The resulting mixture was concentrated under reduced pressure. The residue was purified by reversed-phase flash chromatography with the following conditions: column, C18; mobile phase, acetonitrile in water (0.5% HCOOH), 40% to 50% gradient in 10 min; detector, UV 254 nm to afford 3-[2,6-bis(benzyloxy)pyridin-3-yl]phenol (7.00 g, 16.79%) as a brown solid. MS (ESI) calc'd for (C₂₅H₂₁NO₃) [M+H]⁺, 384.1; found 384.1.

### Step 2: Synthesis of methyl (1r,4r)-4-(3-(2,6-bis(benzyloxy)pyridin-3-yl)phenoxy)cyclohexane-1-carboxylate

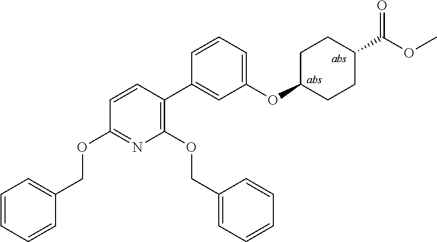 [SMILES: COC(=O)[C@H]1CC[C@H](Oc2cccc(-c3ccc(OCc4ccccc4)nc3OCc3ccccc3)c2)CC1]

To a stirred mixture of 3-[2,6-bis(benzyloxy)pyridin-3-yl]phenol (7.00 g, 18.25 mmol, 1.00 equiv) and methyl (1s,4s)-4-hydroxycyclohexane-1-carboxylate (9.90 mg, 0.06 mmol, 1.20 equiv) in THF (50.00 mL) were added PPh₃ (9.58 g, 36.51 mmol, 2.00 equiv) and DIAD (7.38 g, 36.51 mmol, 2.00 equiv) at 0° C. The resulting mixture was stirred at room temperature overnight under nitrogen atmosphere. The resulting mixture was concentrated under reduced pressure. The residue was purified by silica gel column chromatography, eluted with PE/EA=2/1 to afford methyl (1r,4r)-4-{3-[2,6-bis(benzyloxy)pyridin-3-yl]phenoxy}cyclohexane-1-carboxylate (3.00 g, 31.38%) as a brown oil. MS (ESI) calc'd for (C₃₃H₃₃NO₅) [M+H]⁺, 524.2; found 524.2.

### Step 3: Synthesis of ((1r,4r)-4-(3-(2,6-bis(benzyloxy)pyridin-3-yl)phenoxy)cyclohexyl)methanol

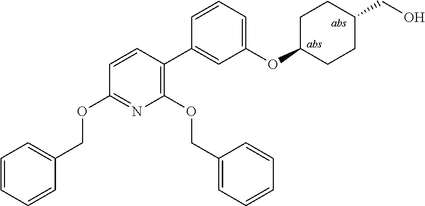 [SMILES: OC[C@H]1CC[C@H](Oc2cccc(-c3ccc(OCc4ccccc4)nc3OCc3ccccc3)c2)CC1]

To a stirred mixture of methyl (1r,4r)-4-{3-[2,6-bis(benzyloxy)pyridin-3-yl]phenoxy}cyclohexane-1-carboxylate (3.00 g, 5.73 mmol, 1.00 equiv) in THF (20 mL) was added LiAlH₄ (0.43 g, 11.46 mmol, 2.00 equiv). The resulting mixture was stirred at room temperature for 1.5 h under nitrogen atmosphere. The mixture was quenched with water. The resulting mixture was extracted with ethyl acetate. The organic layers were combined and concentrated under reduced pressure. The residue was purified by silica gel column chromatography, eluted with PE/EA=1/1 to afford [(1r,4r)-4-{3-[2,6-bis(benzyloxy)pyridin-3-yl]phenoxy}cyclohexyl]methanol (1.30 g, 45.78%) as a white solid. MS (ESI) calc'd for (C₃₂H₃₃NO₄) [M+H]⁺, 496.2; found 496.2.

### Step 4: Synthesis of (1r,4r)-4-(3-(2,6-bis(benzyloxy)pyridin-3-yl)phenoxy)cyclohexane-1-carbaldehyde

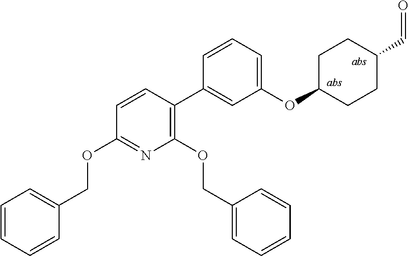 [SMILES: O=C[C@H]1CC[C@H](Oc2cccc(-c3ccc(OCc4ccccc4)nc3OCc3ccccc3)c2)CC1]

To a stirred mixture of [(1r,4r)-4-{3-[2,6-bis(benzyloxy)pyridin-3-yl]phenoxy}cyclohexyl]methanol (1.30 g, 2.62 mmol, 1.00 equiv) in DCM (20 mL) was added Dess-Martin (2.23 g, 5.25 mmol, 2.00 equiv). The resulting mixture was stirred at room temperature for 2 h. The resulting mixture was washed with water. The organic layer was concentrated under reduced pressure. The residue was purified by silica gel column chromatography, eluted with PE/EA=1/1 to afford (1r,4r)-4-{3-[2,6-bis(benzyloxy)pyridin-3-yl]phenoxy}cyclohexane-1-carbaldehyde (900 mg, 69.51%) as a yellow oil. MS (ESI) calc'd for (C₃₂H₃₁NO₄) [M+H]⁺, 494.2; found 494.2.

### Step 5: Synthesis of 2,6-bis(benzyloxy)-3-(3-(((1r,4r)-4-(dimethoxymethyl)cyclohexyl)oxy)phenyl)pyridine

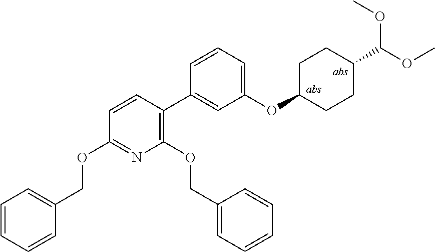 [SMILES: COC(OC)[C@H]1CC[C@H](Oc2cccc(-c3ccc(OCc4ccccc4)nc3OCc3ccccc3)c2)CC1]

To a stirred mixture of (1r,4r)-4-{3-[2,6-bis(benzyloxy)pyridin-3-yl]phenoxy}cyclohexane-1-carbaldehyde (900.00 mg, 1.82 mmol, 1.00 equiv) and trimethyl orthoformate (2.90 g, 27.35 mmol, 15.00 equiv) in MeOH (15 mL) was added H₂SO₄ (2 mL). The resulting mixture was stirred at room temperature for 1 h. The resulting mixture was concentrated under reduced pressure. The residue was purified by silica gel column chromatography, eluted with PE/EA=2/1 to afford 2,6-bis(benzyloxy)-3-(3-{[(1r,4r)-4-(dimethoxymethyl)cyclohexyl]oxy}phenyl)pyridine (800.00 mg, 81.30%) as a yellow solid. MS (ESI) calc'd for (C₃₄H₃₇NO₅) [M+H]⁺, 540.2; found 540.2.

### Step 6: Synthesis of (RS)-3-(3-(((1r,4R)-4-(dimethoxymethyl)cyclohexyl)oxy)phenyl)piperidine-2,6-dione

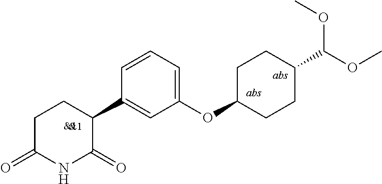 [SMILES: COC(OC)[C@H]1CC[C@H](Oc2cccc([C@@H]3CCC(=O)NC3=O)c2)CC1]

To a stirred solution of 2,6-bis(benzyloxy)-3-(3-{[(1r,4r)-4-(dimethoxymethyl)cyclohexyl]oxy}phenyl) pyridine (800.00 mg, 1.48 mmol, 1.00 equiv) in THF (10 mL) and Isopropanol (2 mL) was added Pd/C (10%, 800.00 mg) and Pd(OH)₂/C (160.00 mg). The resulting mixture was stirred at room temperature for 2 h under hydrogen atmosphere. The solids were filtered off. The filtrate was concentrated under vacuum to afford 3-(3-{[(1r,4r)-4-(dimethoxymethyl) cyclohexyl]oxy}phenyl)piperidine-2,6-dione (400.00 mg, 74.66%) as a white solid. MS (ESI) calc'd for (C₂₀H₂₇NO₅) [M+H]⁺, 362.1; found 362.1.

### Step 7: Synthesis of Title Compound

The resulting solution of 3-(3-{[(1r,4r)-4-(dimethoxymethyl)cyclohexyl]oxy}phenyl)piperidine-2,6-dione (400.00 mg, 1.11 mmol, 1.00 equiv) in FA (5 mL) was stirred at room temperature for 2 h. The resulting mixture was concentrated under reduced pressure to afford (1r,4r)-4-{3-[(3RS)-2,6-dioxopiperidin-3-yl]phenoxy}cyclohexane-1-carbaldehyde (258.10 mg, 70.25%) as a white solid. MS (ESI) calc'd for (C₁₈H₂₁NO₄) [M+1]⁺, 316.15; found, 316.15. ¹H NMR (400 MHz, DMSO-d₆) δ 10.81 (s, 1H), 9.62 (s, 1H), 7.34-7.13 (m, 1H), 6.95-6.57 (m, 3H), 4.38-4.17 (m, 1H), 3.95-3.65 (m, 1H), 2.73-2.60 (m, 1H), 2.47-2.42 (m, 1H), 2.39-2.30 (m, 1H), 2.27-2.16 (m, 1H), 2.12-1.93 (m, 5H), 1.52-1.32 (m, 4H).

### Intermediate 15

### 4-(1-ETHYL-1H-PYRAZOL-5-YL)-1-(6-(2-(METHOXYMETHOXY)PHENYL)PYRIDAZIN-4-YL)PIPERIDINE-4-CARBOXYLIC ACID

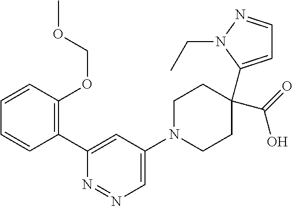 [SMILES: CCn1nccc1C1(C(=O)O)CCN(c2cnnc(-c3ccccc3OCOC)c2)CC1]

### Step 1: Synthesis of tert-butyl 4-cyano-4-(1-ethyl-1H-pyrazol-5-yl)piperidine-1-carboxylate

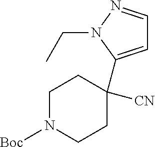 [SMILES: CCn1nccc1C1(C#N)CCN(C(=O)OC(C)(C)C)CC1]

To a solution of 2-(1-ethyl-1H-pyrazol-5-yl)acetonitrile (1.50 g, 11.1 mmol, 1.00 eq) in DMF (15.0 mL) was added NaH (1.11 g, 27.7 mmol, 60% purity, 2.50 eq) at 0° C. under N₂ atmosphere and stirred at 25° C. for 30 mins, then tert-butyl bis(2-chloroethyl)carbamate (3.22 g, 13.3 mmol, 1.20 eq) was added to the mixture, and the mixture was heated to 60° C. and stirred until complete as judged by LCMS. Once complete, the reaction mixture was quenched by addition saturated NH₄Cl solution (50.0 mL) at 0° C. under N₂, and then diluted with water (50 mL) and extracted with EtOAc (40 mL×3). The combined organic layers were washed with brine (50 mL×3), dried over anhydrous Na₂SO₄, filtered and concentrated to afford the title compound a yellow oil (3.38 g) which was taken forward crude without any further purification. LCMS C₁₆H₂₄N₄O₂ requires 304.1, found m/z=305.1 [M+H]⁺.

### Step 2: Synthesis of 4-(1-ethyl-1H-pyrazol-5-yl)piperidine-4-carbonitrile hydrochloride

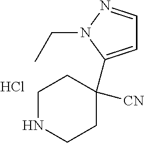

The title compound was prepared as described for Intermediate 1 Step 2, using tert-butyl 4-cyano-4-(1-ethyl-1H-pyrazol-5-yl)piperidine-1-carboxylate (3.38 g, 11.1 mmol), affording the title compound as a yellow solid (3.00 g) which was taken forward crude without any further purification. LCMS Cn H₁₆N₄ requires 204, found m/z=205.1 [M+H]⁺.

### Step 3: Synthesis of 1-(6-chloropyridazin-4-yl)-4-(1-ethyl-1H-pyrazol-5-yl)piperidine-4-carbonitrile

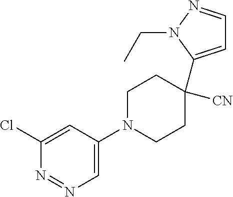 [SMILES: CCn1nccc1C1(C#N)CCN(c2cnnc(Cl)c2)CC1]

The title compound was prepared as described for Intermediate 1 Step 3, using 4-(1-ethyl-1H-pyrazol-5-yl)piperidine-4-carbonitrile hydrochloride (3.00 g, 12.5 mmol), affording the title compound (2.50 g, 77% yield over three steps) as a brown solid. LCMS C₁₅H₁₇ClN₆ requires 316.1, found m/z=317.1 [M+H]⁺.

### Step 4: Synthesis of 4-(1-ethyl-1H-pyrazol-5-yl)-1-(6-(2-(methoxymethoxy)phenyl)pyridazin-4-yl)piperidine-4-carbonitrile

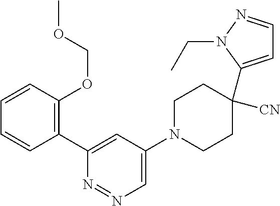 [SMILES: CCn1nccc1C1(C#N)CCN(c2cnnc(-c3ccccc3OCOC)c2)CC1]

The title compound was prepared as described for Intermediate 1 Step 4, using 1-(6-chloropyridazin-4-yl)-4-(1-ethyl-1H-pyrazol-5-yl)piperidine-4-carbonitrile (2.3 g, 7.26 mmol), affording the title compound (3.30 g, 97% yield) as a yellow oil. LCMS C₂₃H₂₆N₆O₂ requires 418.2, found m/z=419.2 [M+H]⁺.

### Step 5: Synthesis of the Title Compound

The title intermediate compound was prepared as described for Intermediate 1 Step 5, using 4-(1-ethyl-1H-pyrazol-5-yl)-1-(6-(2-(methoxymethoxy)phenyl)pyridazin-4-yl)piperidine-4-carbonitrile (3.30 g, 7.07 mmol). An additional adjustment of pH to ~7 with 2N HCl was performed, followed by concentration under reduced pressure. The resulting residue was purified by prep-HPLC (column: Kromasil Eternity XT 250*80 mm*10 um; mobile phase: [H₂O (0.05% NH₄OH)-ACN]; gradient:0%-25% B over 20.0 min) to afford the title compound (2.01 g, 65% yield) as a brown solid. LCMS C₂₃H₂₇N₅O₄ requires 437.2, found m/z=438.2 [M+H]⁺.

### Intermediate 16

### 4-(1,3-DIMETHYL-1H-PYRAZOL-5-YL)-1-(6-(2-(METHOXYMETHOXY)PHENYL)PYRIDAZIN-4-YL)PIPERIDINE-4-CARBOXYLIC ACID

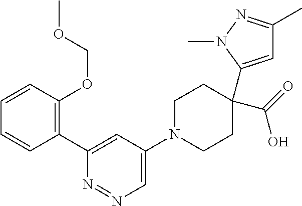 [SMILES: COCOc1ccccc1-c1cc(N2CCC(C(=O)O)(c3cc(C)nn3C)CC2)cnn1]

### Step 1: Synthesis of tert-butyl 4-cyano-4-(1,3-dimethyl-1H-pyrazol-5-yl)piperidine-1-carboxylate

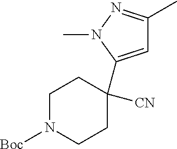 [SMILES: Cc1cc(C2(C#N)CCN(C(=O)OC(C)(C)C)CC2)n(C)n1]

The title compound was prepared as described for Intermediate 15 Step 1, using 2-(1,3-dimethyl-1H-pyrazol-5-yl)acetonitrile (1.50 g, 11.1 mmol), affording the title compound (3.38 g), as a yellow oil which was taken forward crude without further purification. LCMS C₁₆H₂₄N₄O₂ requires 304.2, found m/z=305.2 [M+H]⁺.

### Step 2: Synthesis of 4-(1,3-dimethyl-1H-pyrazol-5-yl)piperidine-4-carbonitrile hydrochloride

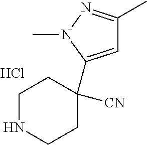

The title compound was prepared as described for the synthesis of Intermediate 1 Step 2, using tert-butyl 4-cyano-4-(1,3-dimethyl-1H-pyrazol-5-yl)piperidine-1-carboxylate (3.38 g, 11.1 mmol), affording the title compound (3.00 g) as a yellow solid which was taken forward crude without any further purification. LCMS C₁₁H₁₆N₄ requires 204.1, found m/z=205.1 [M+H]⁺.

### Step 3: Synthesis of 1-(6-chloropyridazin-4-yl)-4-(1,3-dimethyl-1H-pyrazol-5-yl)piperidine-4-carbonitrile

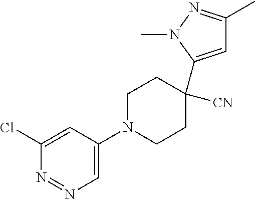 [SMILES: Cc1cc(C2(C#N)CCN(c3cnnc(Cl)c3)CC2)n(C)n1]

The title compound was prepared as described for Intermediate 1 Step 3, using 4-(1,3-dimethyl-1H-pyrazol-5-yl)piperidine-4-carbonitrile hydrochloride (3.00 g, 12.5 mmol), affording the title compound (2.00 g, 57% yield over three steps) as a brown solid. LCMS C₁₅H₁₇ClN₆ requires 316.1, found m/z=317.1 [M+H]⁺.

### Step 4: Synthesis of 4-(1,3-dimethyl-1H-pyrazol-5-yl)-1-(6-(2-(methoxymethoxy)phenyl)pyridazin-4-yl)piperidine-4-carbonitrile

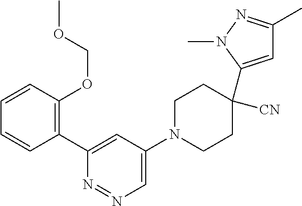 [SMILES: COCOc1ccccc1-c1cc(N2CCC(C#N)(c3cc(C)nn3C)CC2)cnn1]

The title compound was prepared as described for Intermediate 1 Step 4, using 1-(6-chloropyridazin-4-yl)-4-(1,3-dimethyl-1H-pyrazol-5-yl)piperidine-4-carbonitrile (1.80 g, 5.68 mmol), affording the title compound (2.40 g, 91% yield) as a yellow oil. LCMS C₂₃H₂₆N₆O₂ requires 418.2, found m/z=419.2 [M+H]⁺.

### Step 5: Synthesis of Title Compound

The title compound was prepared as described for Intermediate 1 Step 5, using 4-(1,3-dimethyl-1H-pyrazol-5-yl)-1-(6-(2-(methoxymethoxy)phenyl)pyridazin-4-yl)piperidine-4-carbonitrile (2.40 g, 5.16 mmol). An additional adjustment of pH to ~7 with 2N HCl was performed, followed by concentration under reduced pressure. The resulting residue was purified by prep-HPLC (column: Kromasil Eternity XT 250*80 mm*10 um; mobile phase: [H₂O (0.05% NH₄OH)-ACN]; gradient:0%-25% B over 20.0 min) to afford the title compound (1.52 g, 67% yield) as a brown solid. LCMS C₂₃H₂₇N₅O₄ requires 437.2, found m/z=438.2 [M+H]⁺.

### Intermediate 17

### 1-(6-(2-(METHOXYMETHOXY)PHENYL)PYRIDAZIN-4-YL)-4-(THIAZOL-4-YL)PIPERIDINE-4-CARBOXYLIC ACID

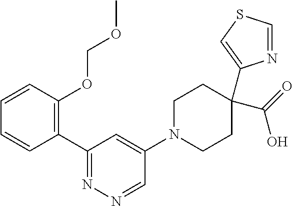 [SMILES: COCOc1ccccc1-c1cc(N2CCC(C(=O)O)(c3cscn3)CC2)cnn1]

### Step 1: Synthesis of 4-(thiazol-4-yl)piperidine-4-carbonitrile

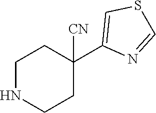 [SMILES: N#CC1(c2cscn2)CCNCC1]

To a solution of tert-butyl 4-cyano-4-(thiazol-4-yl)piperidine-1-carboxylate (CAS 1522499-80-4, Enamine) (1.86 g, 6.34 mmol, 1.00 eq) in HCl/EtOAc (2 M, 20.00 mL, 6.31 eq) was stirred at 25° C. for 2 hrs. The mixture was concentrated in vacuum. 4-(thiazol-4-yl)piperidine-4-carbonitrile (1.46 g, crude, HCl) was obtained as a brown oil. LCMS: C₉H₁₁N₃S requires 193.1, found: m/z=194.0 [M+H]⁺.

### Step 2: Synthesis of 1-(6-chloropyridazin-4-yl)-4-(thiazol-4-yl)piperidine-4-carbonitrile

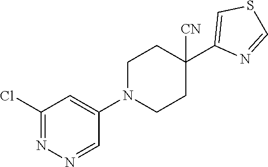 [SMILES: N#CC1(c2cscn2)CCN(c2cnnc(Cl)c2)CC1]

To a solution of 4-(thiazol-4-yl)piperidine-4-carbonitrile (1.46 g, 6.36 mmol, 1.00 eq, HCl) and 3,5-dichloropyridazine (1.42 g, 9.53 mmol, 1.50 eq) in IPA (120.00 mL) was added DIPEA (3.29 g, 25.40 mmol, 4.43 mL, 4.00 eq) under N₂ atmosphere, and the mixture was stirred at 70° C. for 12 hrs. The mixture was concentrated to remove most IPA in vacuum. The crude product was purified by reversed-phase HPLC (column: Kromasil Eternity XT 250*80 mm*10 um; mobile phase: [water (ammonia hydroxide v/v)-ACN]; gradient:10%-40% B over 20 min). 1-(6-chloropyridazin-4-yl)-4-(thiazol-4-yl)piperidine-4-carbonitrile (1.43 g, 4.12 mmol, 44.0% yield, 88.0% purity) was obtained as a brown solid. LCMS: C₁₃H₁₂ClN₅S requires 305.1, found: m/z=306.0 [M+H]⁺.

### Step 3: Synthesis of 1-(6-(2-(methoxymethoxy)phenyl)pyridazin-4-yl)-4-(thiazol-4-yl)piperidine-4-carbonitrile

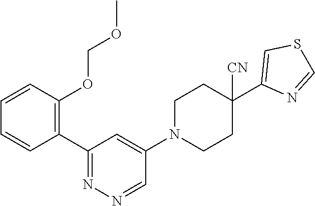 [SMILES: COCOc1ccccc1-c1cc(N2CCC(C#N)(c3cscn3)CC2)cnn1]

To a solution of 1-(6-chloropyridazin-4-yl)-4-(thiazol-4-yl)piperidine-4-carbonitrile (1.17 g, 3.37 mmol, 1.00 eq), (2-(methoxymethoxy)phenyl)boronic acid (1.23 g, 6.73 mmol, 2.00 eq) and K₂CO₃ (1.40 g, 10.10 mmol, 3.00 eq) in dioxane (50.00 mL) and H₂O (10.00 mL) was added Pd(dppf)Cl₂·CH₂Cl₂ (274.00 mg, 336.00 μmol, 0.10 eq) at 25° C. under N₂ atmosphere, and the mixture was stirred at 110° C. for 6 hrs. The reaction mixture was cooled to 40° C. and diluted with water (100 mL) and extracted with EtOAc, and the combined organic layers were washed with brine, dried over Na₂SO₄ and concentrated to obtain crude product under vacuum. The residue was triturated with MTBE/Ethyl acetate (10/1, 10.00 mL), at 25° C. for 20 mins, filtered and the filter cake was washed with MTBE (5.00 mL*2), dried in vacuum. 1-(6-(2-(methoxymethoxy)phenyl)pyridazin-4-yl)-4-(thiazol-4-yl)piperidine-4-carbonitrile (1.20 g, 2.64 mmol, 78.3% yield, 89.6% purity) was obtained as a black brown solid. LCMS: C₂₁H₂₁N₅O₂S requires 407.1, found: m/z=408.2 [M+H]⁺.

### Step 4: Synthesis of the Title Compound

To a solution of 1-(6-(2-(methoxymethoxy)phenyl)pyridazin-4-yl)-4-(thiazol-4-yl)piperidine-4-carbonitrile (1.20 g, 2.64 mmol, 1.00 eq) in MeOH (10.00 mL), THF (10.00 mL) and H₂O (10.00 mL) was added NaOH (1.58 g, 39.58 mmol, 15.00 eq) at 25° C. Then the mixture reaction was stirred at 80° C. for 12 hrs. The mixture was concentrated in vacuum, adjusted pH to about 7.00 with 2N HCl, concentrated in vacuum. The crude product was purified by reversed-phase HPLC (column: Waters Xbridge 150*25 mm*um; mobile phase: [water (NH3H₂O)-ACN]; gradient:0%-30% B over 11 min). 1-(6-(2-(methoxymethoxy)phenyl)pyridazin-4-yl)-4-(thiazol-4-yl)piperidine-4-carboxylic acid (803.00 mg, 1.88 mmol, 71.2% yield, 99.8% purity) was obtained as a red solid. LCMS: C₂₁H₂₂N₄O₄S requires 426.1, found: m/z=427.2 [M+H]⁺.

### Intermediate 18

### (R)-2-(1-(4-(2,6-DIOXOPIPERIDIN-3-YL)-3-FLUOROPHENYL)PIPERIDIN-4-YL)ACETALDEHYDE

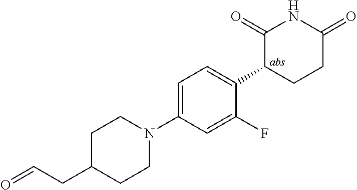 [SMILES: O=CCC1CCN(c2ccc([C@H]3CCC(=O)NC3=O)c(F)c2)CC1]

### Step 1: Synthesis of 2,6-bis(benzyloxy)-3-(4-bromo-2-fluorophenyl)pyridine

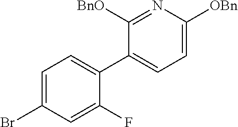 [SMILES: Fc1cc(Br)ccc1-c1ccc(OCc2ccccc2)nc1OCc1ccccc1]

To a solution of 4-bromo-2-fluoro-1-iodobenzene (200 g, 664 mmol, 1.0 eq) and 2,6-bis(benzyloxy)-3-(4,4,5,5-tetramethyl-1,3,2-dioxaborolan-2-yl)pyridine (305 g, 731 mmol, 1.1 eq) in 1,4-dioxane (2.0 L) and H₂O (200 mL) was added K₂CO₃ (275 g, 1.99 mol, 3.0 eq) and Pd(dppf)Cl₂-DCM (27.1 g, 33.2 mmol, 0.05 eq). The mixture was stirred at 80° C. for 4 h. The reaction was allowed to cooled to room temperature, added water (1.0 L) and extracted by EtOAc (2.0 L×2), the combined organic phase was washed with brine (1.5 L×2), dried over Na₂SO₄, filtered and concentrated in vacuo. The crude was purified by silica gel column chromatography (petroleum ether/EtOAc=50:1) to yield the product (220 g, yield 71.3%) as a white solid. ¹H NMR (300 MHz, DMSO-d₆): δ 7.72-7.56 (m, 2H), 7.51-7.22 (m, 12H), 6.56 (d, J=8.1 Hz, 1H), 3.32 (s, 4H). LCMS: 464.2, 466.0 ([M+H]+).

### Step 2: Synthesis of 2-(1-(4-(2,6-bis(benzyloxy)pyridin-3-yl)-3-fluorophenyl)piperidin-4-yl)ethan-1-ol

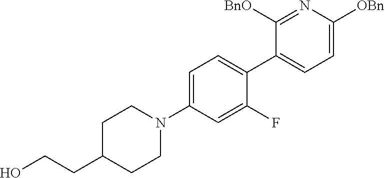 [SMILES: OCCC1CCN(c2ccc(-c3ccc(OCc4ccccc4)nc3OCc3ccccc3)c(F)c2)CC1]

To a solution of 2,6-bis(benzyloxy)-3-(4-bromo-2-fluorophenyl)pyridine (245 g, 527 mmol, 1.0 eq) and 2-(piperidin-4-yl)ethan-1-ol (75.0 g, 580 mmol, 1.1 eq) in Toluene (2.5 L) was added t-BuONa (101 g, 1.06 mol, 2.0 eq), BINAP (64.7 g, 104 mmol, 0.2 eq) and Pd(OAc)₂ (12.3 g, 54.9 mmol, 0.1 eq). The mixture was stirred at 100° C. for 4 h. The reaction was allowed to cooled to room temperature, added water (1.0 L) and extracted by EtOAc (2.0 L×2), the combined organic phase was washed with brine (1.5 L×2), dried over Na₂SO₄, filtered and concentrated in vacuo. The crude was purified by silica gel column chromatography (petroleum ether/EtOAc=10:1~3:1) to give the product (83.0 g, yield 30.6%) as a yellow solid. ¹H NMR (400 MHz, CDCl₃): δ 7.52 (dd, J=8.0, 0.8 Hz, 1H), 7.48-7.20 (m, 11H), 6.80-6.60 (m, 2H), 6.44 (d, J=8.0 Hz, 1H), 5.40 (s, 2H), 5.33 (s, 2H), 3.83-3.67 (m, 4H), 2.87-2.70 (m, 2H), 1.89-1.77 (m, 2H), 1.70-1.56 (m, 3H), 1.48-1.30 (m, 2H).

### Step 3: Synthesis of 3-(2-fluoro-4-(4-(2-hydroxyethyl)piperidin-1-yl)phenyl)piperidine-2,6-dione

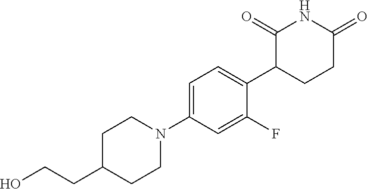 [SMILES: O=C1CCC(c2ccc(N3CCC(CCO)CC3)cc2F)C(=O)N1]

The reaction mixture of 2-(1-(4-(2,6-bis(benzyloxy)pyridin-3-yl)-3-fluorophenyl)piperidin-4-yl)ethan-1-ol (63.0 g, 37.2 mmol, 1.0 eq), Pd/C (15.7 g) and Pd(OH)₂ (15.7 g) in 1,4-dioxane (650 mL) was stirred under H₂ at 60° C. for 36 h. LCMS and TLC showed the reaction was completed. The mixture was filtered, and the filtrate was concentrated to give the crude, which was triturated with EtOAc (250 mL) at 25° C. for 15 min and filtered to give the product (25.0 g, yield 60.8%) as a grey solid. ¹H NMR (400 MHz, DMSO-d₆): δ 10.79 (brs, 1H), 7.03 (t, J=8.8 Hz, 1H), 6.77-6.62 (m, 2H), 4.35 (t, J=4.8 Hz, 1H), 3.87 (dd, J=12.3, 4.8 Hz, 1H), 3.79-3.60 (m, 2H), 3.50-3.40 (m, 2H), 2.80-2.60 (m, 3H), 2.22-2.08 (m, 1H), 2.07-1.90 (m, 1H), 1.80-1.63 (m, 2H), 1.62-1.48 (m, 1H), 1.38 (q, J=6.8 Hz, 2H), 1.30-1.10 (m, 2H). LCMS: 335.2 ([M+H]⁺).

### Step 4: Synthesis of the Title Compound

To a solution of 3-(2-fluoro-4-(4-(2-hydroxyethyl)piperidin-1-yl)phenyl)piperidine-2,6-dione (31.5 g, 94.2 mmol, 1.0 eq) in DMSO (315 mL) was added IBX (53.1 g, 188 mmol, 2.0 eq), then the reaction mixture was stirred at room temperature for 3 h. LCMS showed the reaction was completed. The reaction mixture was dropwise into saturated Na₂CO₃ aqueous solution (600 mL) at 0° C. and extracted with DCM (500 mL×2). The combined organic phases were washed with saturated Na₂S₂O₃ aqueous solution (300 mL×3), dried over Na₂SO₄ and concentrated to give a crude. The crude was added DCM (400 mL) and NaHSO₃ aqueous solution (200 mL, 1.2M), and then stirred at room temperature for 1 h. After that, the aqueous phase was extracted with DCM (200 mL×2). The aqueous phase was added Na₂CO₃ aqueous solution (600 mL, 0.6M) and extracted with DCM (500 mL×2), the combined organic phase was washed with brine (200 mL×3), dried over Na₂SO₄ and concentrated in vacuo to give the product (11.3 g, yield 36.1%) as a yellow solid. ¹H NMR (300 MHz, DMSO-d₆): δ 10.80 (brs, 1H), 9.69 (brs, 1H), 7.06 (t, J=8.7 Hz, 1H), 6.80-6.60 (m, 2H), 3.87 (dd, J=12.3, 4.8 Hz, 1H), 3.80-3.60 (m, 2H), 2.82-3.62 (m, 3H), 2.46-2.34 (m, 2H), 2.24-1.87 (m, 4H), 1.80-1.60 (m, 2H), 1.40-1.16 (m, 2H). LCMS: 333.2 ([M+H]+).

### Intermediate 19

### 2-(1-(4-(2,6-DIOXOPIPERIDIN-3-YL)-3,5-DIFLUOROPHENYL)PIPERIDIN-4-YL)ACETALDEHYDE

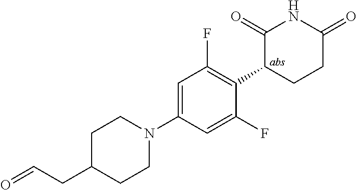 [SMILES: O=CCC1CCN(c2cc(F)c([C@H]3CCC(=O)NC3=O)c(F)c2)CC1]

### Step 1: Synthesis of 2,6-bis(benzyloxy)-3-(4-bromo-2,6-difluorophenyl)pyridine

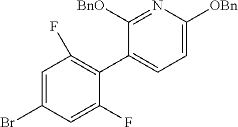 [SMILES: Fc1cc(Br)cc(F)c1-c1ccc(OCc2ccccc2)nc1OCc1ccccc1]

To a solution of 5-bromo-1,3-difluoro-2-iodobenzene (167 g, 525 mmol, 1.0 eq) in DMF (3344 mL) and H₂O (334 mL) was added 2,6-bis(benzyloxy)-3-(4,4,5,5-tetramethyl-1,3,2-dioxaborolan-2-yl)pyridine (225 g, 539.4 mmol, 1.03 eq), CsF (159 g, 1051.0 mmol, 2.0 eq) and Pd(dtbpf)Cl₂ (33.8 g, 52.5 mmol, 0.1 eq) at room temperature under N₂. The mixture was purged with N₂ three times. The reaction mixture was stirred at 85° C. for 12 h. HPLC showed the reaction was complete. After cooled to room temperature, water (3000 mL) was added. The mixture was extracted with EtOAc (2000 mL×3). The combined organic phases were washed with brine (1500 mL×5), dried over Na₂SO₄ and concentrated to give crude product. The residue was triturated with petroleum ether (450 mL) to afford Compound 2 (115.0 g, 45.5%) as a white solid. 1H NMR (300 MHz, CDCl3): δ 7.38-7.35 (m, 1H), 7.33-7.13 (m, 10H), 7.06-6.99 (m, 2H), 6.38 (d, J=8.1 Hz, 1H), 5.27-5.23 (m, 4H).

### Step 2: Synthesis of 2-(1-(4-(2,6-bis(benzyloxy)pyridin-3-yl)-3,5-difluorophenyl)piperidin-4-yl)ethan-1-ol

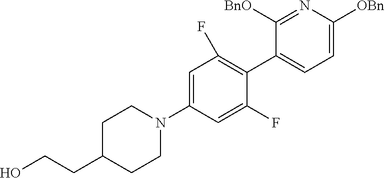 [SMILES: OCCC1CCN(c2cc(F)c(-c3ccc(OCc4ccccc4)nc3OCc3ccccc3)c(F)c2)CC1]

To a solution of 2,6-bis(benzyloxy)-3-(4-bromo-2,6-difluorophenyl)pyridine (46.0 g, 95.4 mmol, 1.0 eq) in toluene (460 mL) was added 2-(piperidin-4-yl)ethan-1-ol (13.6 g, 104.9 mmol, 1.1 eq), t-BuONa (18.3 g, 190 mmol, 2.0 eq), BINAP (11.9 g, 19.1 mmol, 0.2 eq) and Pd(OAc)₂ (2.1 g, 9.5 mmol, 0.1 eq) at room temperature under N₂. The mixture was purged with N₂ three times. The reaction mixture was stirred at 110° C. for 12 h. HPLC showed the reaction was complete. After cooled to room temperature, water (300 mL) was added. The mixture was extracted with EtOAc (300 mL×2). The combined organic phases were washed with brine (400 mL×2), dried over Na₂SO₄ and concentrated to give crude product, which was purified by silica gel column chromatography with (petroleum ether:EtOAc=20:1) to give Compound 3 (26.0 g, 51.3%) as a white oil. ¹H NMR (400 MHz, CDCl₃): δ 7.42-7.18 (m, 11H), 6.41-6.35 (m, 3H), 5.31 (s, 2H), 5.24 (s, 2H), 5.22 (s, 2H), 3.66-3.60 (m, 4H), 2.74-2.68 (m, 2H), 1.74-1.70 (m, 2H), 1.50-1.44 (m, 2H), 1.32-1.24 (m, 2H).

### Step 3: Synthesis of 3-(2,6-difluoro-4-(4-(2-hydroxyethyl)piperidin-1-yl)phenyl)piperidine-2,6-dione

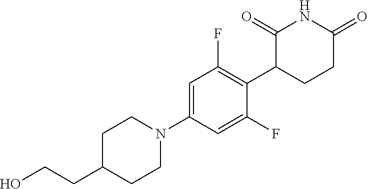 [SMILES: O=C1CCC(c2c(F)cc(N3CCC(CCO)CC3)cc2F)C(=O)N1]

To a solution of 2-(1-(4-(2,6-bis(benzyloxy)pyridin-3-yl)-3,5-difluorophenyl)piperidin-4-yl)ethan-1-ol (23.6 g, 44.5 mmol, 1.0 eq) in dioxane (236 mL) was added Pd/C (11.8 g) and Pd(OH)₂ (11.8 g) at room temperature. The mixture was purged with H₂ three times. The mixture was stirred at 40° C. for 24 h. HPLC showed the reaction was complete. The reaction mixture was filtered and the filtrate was concentrated to give crude product, which was triturated with MTBE (40 mL) to afford the desired product (8.8 g, 56.1%) as a blue solid. ¹H NMR (300 MHz, DMSO-d₆): δ 10.85 (s, 1H), 6.62 (d, J=12.8 Hz, 2H), 4.36 (t, J=5.1 Hz, 1H), 4.06-4.00 (m, 1H), 3.75-3.71 (m, 2H), 3.49-3.43 (m, 2H), 2.84-2.65 (m, 3H), 2.53-2.51 (m, 1H), 2.15-1.92 (m, 2H), 1.72-1.68 (m, 2H), 1.61-1.51 (m, 1H), 1.41-1.34 (m, 2H), 1.22-1.09 (m, 2H). LCMS: 353.2 ([M+H]⁺).

### Step 4: Synthesis of the Title Compound

To a solution of 3-(2,6-difluoro-4-(4-(2-hydroxyethyl)piperidin-1-yl)phenyl)piperidine-2,6-dione (8.8 g, 25.0 mmol, 1.0 eq) in DMSO (85 mL) was added IBX (10.6 g, 37.5 mmol, 1.5 eq). The reaction mixture was stirred at room temperature for 3 h. A saturated sodium thiosulfate solution (100 mL) was added, and the mixture was extracted with EtOAc (100 mL×2). The combined organic phases were washed with brine (100 mL×4), dried over Na₂SO₄ and concentrated to give crude product. The crude product was triturated with MTBE (30 mL) and filtered to give the desired product (6.0 g, 68.6%) as a yellow solid. ¹H NMR (300 MHz, DMSO-d₆): δ 10.85 (s, 1H), 9.70-9.69 (m, 1H), 6.63 (s, 1H), 6.59 (s, 1H), 4.06-4.01 (m, 1H), 3.75-3.71 (m, 2H), 2.75-2.72 (m, 3H), 2.54-2.53 (m, 1H), 2.41-2.38 (m, 2H), 2.11-1.92 (m, 3H), 1.71-1.67 (m, 2H), 1.30-1.16 (m, 2H). LCMS: 351.1 ([M+H]+).

### Intermediate 20

### 1-(1-(6-(2-HYDROXYPHENYL)PYRIDAZIN-4-YL)-4-(1-METHYL-1H-PYRAZOL-4-YL)PIPERIDIN-4-YL)ETHAN-1-ONE

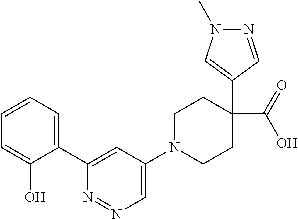 [SMILES: Cn1cc(C2(C(=O)O)CCN(c3cnnc(-c4ccccc4O)c3)CC2)cn1]

### Step 1: Synthesis of 4-(chloromethyl)-1-methyl-1H-pyrazole

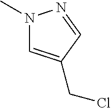

To a solution of (1-methyl-1H-pyrazol-4-yl)methanol (25.0 g, 222 mmol, 1.00 eq) in DCM (250 mL) was added SOCl₂ (66.3 g, 557 mmol, 40.4 mL) dropwise. The mixture was stirred at 25° C. for 12 hrs. The mixture was directly filtered and concentrated under vacuum to give the desired product (34.1 g, crude) as a white solid. m/z=131.2 (M+H)⁺

### Step 2: Synthesis of 4-(chloromethyl)-1-methyl-1H-pyrazole

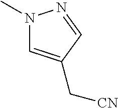

To a solution of 4-(chloromethyl)-1-methyl-1H-pyrazole (33.9 g, 259 mmol) in ACN (180 mL) and H₂O (37.0 mL) was added KCN (27.9 g, 428 mmol, 18.3 mL) at 25° C. The mixture was heated to 50° C. and stirred for 12 hrs. The mixture was then cooled down to 25° C. and adjusted the pH to 9 with saturated NaHCO₃ aqueous solution (800 mL). Then the mixture was extracted with EtOAc (900 mL*3). The organic layers were washed with brine (800 mL*2), dried over Na₂SO₄, filtered and concentrated to give the product. The residue was purified by column chromatography (SiO₂, petroleum ether/Ethyl acetate=20/1 to 0/1, R_{f}=0.20, petroleum ether/Ethyl acetate=5/1, PMA). The mixture was concentrated to give the desired product (23.0 g, 186 mmol, 71.9% yield) as yellow oil. m/z=122.2 (M+H)⁺

### Step 3: Synthesis of tert-butyl 4-cyano-4-(1-methyl-1H-pyrazol-4-yl)piperidine-1-carboxylate

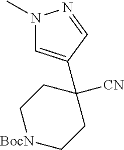 [SMILES: Cn1cc(C2(C#N)CCN(C(=O)OC(C)(C)C)CC2)cn1]

To a solution of 2-(1-methyl-1H-pyrazol-4-ylacetonitrile (23.0 g, 186 mmol) in DMF (460 mL) was added NaH (18.6 g, 467 mmol) at 0° C., the mixture was stirred at 0° C. for 0.5 hr. Then tert-butyl bis(2-chloroethyl)carbamate (49.7 g, 205 mmol, 1.10 eq) was added, the mixture was heated to 60° C. and stirred for 2 hrs. The mixture was cooled down to 25° C., and poured into a saturated NH₄Cl aqueous solution (1.00 L), and then extracted with EtOAc (800 mL*3). The combined organic phases were washed with brine (500 mL), dried over Na₂SO₄, filtered and concentrated under vacuum to give the product (52.1 g, 144 mmol, 77.2% yield) as a black brown oil. m/z=291.3 (M+H)⁺

### Step 4: Synthesis of 4-(1-methyl-1H-pyrazol-4-yl)piperidine-4-carbonitrile

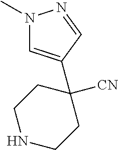

To a solution of tert-butyl 4-cyano-4-(1-methyl-1H-pyrazol-4-yl)piperidine-1-carboxylate (52.1 g, 128 mmol) in EtOAc (100 mL) was added HCl/EtOAc (2 M, 961 mL) at 25° C. and the mixture was stirred for 12 hrs. The mixture was filtered and the filtrate was concentrated under vacuum to give the desired product (22.5 g, 111 mmol, 78.3% yield) as a black brown solid. m/z=191.2 (M+H)⁺

### Step 5: Synthesis of 1-(6-chloropyridazin-4-yl)-4-(1-methyl-1H-pyrazol-4-yl)piperidine-4-carbonitrile

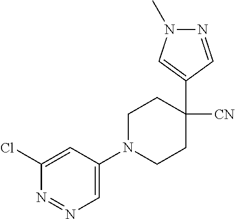 [SMILES: Cn1cc(C2(C#N)CCN(c3cnnc(Cl)c3)CC2)cn1]

To a solution of 4-(1-methyl-1H-pyrazol-4-yl)piperidine-4-carbonitrile (22.5 g, 93.4 mmol) and 3,5-dichloropyridazine (16.7 g, 112 mmol) in IPA (600 mL) was added DIEA (60.4 g, 467 mmol, 81.4 m) at 25° C. and the resulting mixture was stirred at 70° C. for 12 hrs. The mixture was poured into H₂O (2.00 L) and extracted with ethyl acetate (900 mL*3). The combined organic phase was washed with brine (2.00 L), dried over Na₂SO₄, filtered and concentrated under vacuum to give the desired product (28.5 g, 83.0 mmol, 88.8% yield) as a yellow solid. m/z=303.2 (M+H)⁺

### Step 6: 1-(6-(2-hydroxyphenyl)pyridazin-4-yl)-4-(1-methyl-1H-pyrazol-4-yl)piperidine-4-carbonitrile

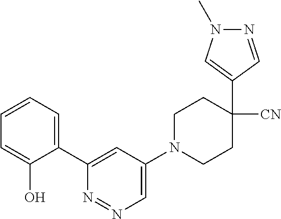 [SMILES: Cn1cc(C2(C#N)CCN(c3cnnc(-c4ccccc4O)c3)CC2)cn1]

To a solution of 1-(6-chloropyridazin-4-yl)-4-(1-methyl-1H-pyrazol-4-yl)piperidine-4-carbonitrile (8.50 g, 27.6 mmol), (2-hydroxyphenyl)boronic acid (7.64 g, 55.3 mmol), K₂CO₃ (11.4 g, 83.0 mmol) in dioxane (85.0 mL) and H₂O (17.0 mL) was added Pd(dppf)Cl₂ (4.05 g, 5.54 mmol) at 25° C., the mixture was stirred at 110° C. for 12 hrs. The mixture was poured into H₂O (100 mL) and extracted with ethyl acetate (100 mL*3). The combined organic phases were washed with brine (100 mL), dried over Na₂SO₄, the mixture was concentrated under vacuum. The residue was purified by column chromatography (SiO₂, Dichloromethane/Methanol=20/1 to 1/1, Dichloromethane/Methanol=20/1, R_{f}=0.15, PMA) to afford the desired compound (3.50 g, 9.56 mmol, 34.5% yield) as a white solid. m/z=361.1 (M+H)⁺

### Step 7: Synthesis of the Title Compound

To a solution of 1-(6-(2-hydroxyphenyl)pyridazin-4-yl)-4-(1-methyl-1H-pyrazol-4-yl)piperidine-4-carbonitrile (3.50 g, 9.24 mmol) in MeOH (20.0 mL), THF (20.0 mL), H₂O (20.0 mL) was added NaOH (3.70 g, 92.4 mmol) at 25° C. and then the resulting mixture was stirred at 80° C. for 12 hrs. Once complete, the mixture was concentrated under vacuum. Then the mixture was adjusted to pH=5 with 1M HCl and extracted with ethyl acetate (500 mL*3). The combined organic phases were washed with brine (300 mL), dried over Na₂SO₄, filtered and concentrated under vacuum. The residue was purified by prep-HPLC to afford the desired product (2.10 g, 5.51 mmol, 59.6% yield) as a yellow solid.

### Intermediate 21

### 1-{6-[2-(METHOXYMETHOXY)PHENYL]PYRIDAZIN-4-YL}-4-[2-(OXAN-4-YL)PYRAZOL-3-YL]PIPERIDINE-4-CARBOXYLIC ACID

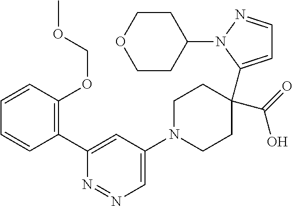 [SMILES: COCOc1ccccc1-c1cc(N2CCC(C(=O)O)(c3ccnn3C3CCOCC3)CC2)cnn1]

### Step 1: Synthesis of 1-(tetrahydro-2H-pyran-4-yl)-1H-pyrazole

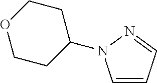 [SMILES: c1cnn(C2CCOCC2)c1]

To a solution of tetrahydro-2H-pyran-4-ol (20.0 g, 196 mmol, 19.6 mL, 1.00 eq) and pyrazole (13.3 g, 196 mmol, 1.00 eq) in Tol (200 mL) was added (cyanomethylene)tributylphosphorane (70.9 g, 294 mmol, 1.50 eq). The mixture was stirred at 115° C. for 12 hrs. The reaction mixture was cooled to 25° C. and washed with brine (500 mL*3), dried over Na₂SO₄, filtered and concentrated under reduced pressure. The crude was purified by prep-HPLC (column: Phenomenex luna c18 250 mm*100 mm*10 um; mobile phase: [H₂O (10 mM NH4HCO3)-ACN]; gradient:10%-30% B over 20.0 min), extracted with DCM (600 mL*2), dried over Na₂SO₄, filtered and concentrated under reduced pressure (below 40° C.) to get 1-(tetrahydro-2H-pyran-4-yl)-1H-pyrazole (9.50 g, 62.4 mmol, 31.9% yield, 100% purity) as an off-white solid. LCMS: C₈H₁₂N₂O requires 152.1, found: m/z=153.1 (M+H)⁺. ¹H NMR (400 MHz, CDCl₃) δ 7.50 (d, J=1.2 Hz, 1H), 7.42 (d, J=2.4 Hz, 1H), 6.25 (t, J=2.0 Hz, 1H), 4.41-4.29 (m, 1H), 4.19-4.03 (m, 2H), 3.62-3.46 (m, 2H), 2.19-2.01 (m, 4H)

### Step 2: Synthesis of 5-iodo-1-(tetrahydro-2H-pyran-4-yl)-1H-pyrazole

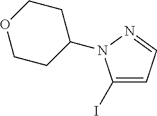 [SMILES: Ic1ccnn1C1CCOCC1]

To a solution of 1-(tetrahydro-2H-pyran-4-yl)-1H-pyrazole (8.50 g, 55.9 mmol, 1.00 eq) in THF (85.0 mL) was added n-BuLi (2.5 M, 33.5 mL, 1.50 eq) drop-wise at −70~−60° C. under N₂, then stirred at −70° C. for 1 hr. A solution of I₂ (21.3 g, 83.8 mmol, 1.50 eq) in THF (85.0 mL) was added drop-wise and stirred at −70~−60° C. for another 1 hr. The mixture was quenched by 10% Na₂SO₃ solution (300 mL) extracted with EtOAc (300 mL*2), the combined organic layer was washed with brine (300 mL), dried over Na₂SO₄, filtered and concentrated under reduced pressure. The crude was purified by prep-HPLC (column: Phenomenex luna c18 250 mm*100 mm*10 um; mobile phase: [H₂O (0.1% TFA)-ACN]; gradient:30%-50% B over 20.0 min), concentrated to remove most ACN, then extracted with EtOAc (300 mL*3), the combined organic layer was washed with brine (500 mL), dried over Na₂SO₄, filtered and concentrated to obtain 5-iodo-1-(tetrahydro-2H-pyran-4-yl)-1H-pyrazole (11.2 g, 65.9% yield) as a yellow solid. LCMS: C₈H₁₁IN₂O requires 278.0, found: m/z=279.0 (M+H)⁺. ¹H NMR: (400 MHz, CDCl₃) δ 7.50 (d, J=1.2 Hz, 1H), 7.42 (d, J=2.4 Hz, 1H), 6.25 (t, J=2.0 Hz, 1H), 4.41-4.29 (m, 1H), 4.19-4.03 (m, 2H), 3.62-3.46 (m, 2H), 2.19-2.01 (m, 4H)

### Step 3: Synthesis of 2-(1-(tetrahydro-2H-pyran-4-yl)-1H-pyrazol-5-yl)acetonitrile

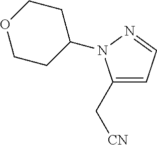 [SMILES: N#CCc1ccnn1C1CCOCC1]

Five batches were carried out in parallel.

To a mixture of 5-iodo-1-(tetrahydro-2H-pyran-4-yl)-1H-pyrazole (1.00 g, 3.60 mmol, 1.00 eq), 4-(4,4,5,5-tetramethyl-1,3,2-dioxaborolan-2-yl)isoxazole (1.05 g, 5.39 mmol, 1.50 eq) and KF (627 mg, 10.8 mmol, 3.00 eq) in dioxane (10.0 mL) and H₂O (2.50 mL) was added 4-ditert-butylphosphanyl-N,N-dimethyl-aniline; dichloropalladium (CAS: 26-37-60) (127 mg, 180 μmol, 127 μL, 0.05 eq) under N₂. The resulting mixture was degassed and purged with N₂ for 3 times and then the mixture was stirred at 80~90° C. for 12 hrs under N₂ atmosphere. The reaction mixture was cooled to 25° C. and concentrated under reduced pressure. The residue was purified by column chromatography (SiO₂, petroleum ether/Ethyl acetate=10/1 to 1/1, petroleum ether/Ethyl acetate=1/1, R_{f}=0.4), concentrated to get a brown oil, which was further purified by prep-HPLC (column: Welch Ultimate XB—CN 250*70 mm*10 um; mobile phase: [Heptane-EtOH (0.1% NH₃H₂O)]; gradient:5%-45% B over 15.0 min), concentrated under reduced pressure to get 2-(1-(tetrahydro-2H-pyran-4-yl)-1H-pyrazol-5-yl)acetonitrile (2.30 g, 11.9 mmol, 55.1% yield, 98.8% purity) as a yellow oil. LCMS: C₁₀H₁₃N₃O requires 191.1, found: m/z=192.1 (M+H)⁺. ¹H NMR: (400 MHz, CDCl₃) δ 7.52 (d, J=1.6 Hz, 1H), 6.28 (d, J=1.6 Hz, 1H), 4.27-4.10 (m, 3H), 3.81 (s, 2H), 3.60-3.50 (m, 2H), 2.40-2.30 (m, 2H), 1.96-1.83 (m, 2H)

### Step 4: Synthesis of tert-butyl 4-cyano-4-(1-(tetrahydro-2H-pyran-4-yl)-1H-pyrazol-5-yl)piperidine-1-carboxylate

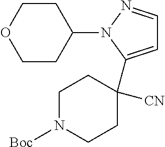 [SMILES: CC(C)(C)OC(=O)N1CCC(C#N)(c2ccnn2C2CCOCC2)CC1]

To a solution of 2-(1-(tetrahydro-2H-pyran-4-yl)-1H-pyrazol-5-yl)acetonitrile (2.30 g, 12.0 mmol, 1.00 eq) in DMF (20.0 mL) was added NaH (1.20 g, 30.1 mmol, 60% purity, 2.50 eq) at 0-10° C. under N₂ atmosphere and stirred at 25° C. for 30 mins, then a solution of tert-butyl bis(2-chloroethyl)carbamate (3.49 g, 14.4 mmol, 1.20 eq) in DMF (3.00 mL) was added to the mixture, and the mixture was heated to 60° C. and stirred at 60° C. for 2 hrs. The reaction mixture was quenched by addition saturated NH₄Cl solution (100 mL) at 0-10° C. under N₂, and extracted with EtOAc (50.0 mL*3). The combined organic layers were washed with brine (30.0 mL*3), dried over Na₂SO₄, filtered and concentrated under vacuum to get tert-butyl 4-cyano-4-(1-(tetrahydro-2H-pyran-4-yl)-1H-pyrazol-5-yl)piperidine-1-carboxylate (4.5 g, crude) as a yellow solid. LCMS: C₁₉H₂₈N₄O₃ requires 360.2, found: m/z=361.3 (M+H)⁺.

### Step 5: Synthesis of 4-(1-(tetrahydro-2H-pyran-4-yl)-1H-pyrazol-5-yl)piperidine-4-carbonitrile hydrochloride

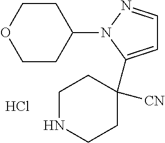 [SMILES: N#CC1(c2ccnn2C2CCOCC2)CCNCC1 ; Cl]

A mixture of tert-butyl 4-cyano-4-(1-(tetrahydro-2H-pyran-4-yl)-1H-pyrazol-5-yl)piperidine-1-carboxylate (4.50 g, 12.5 mmol, 1.00 eq) in HCl/EtOAc (2 M, 50.0 mL) was stirred at 25° C. for 1 hr. The mixture was concentrated under reduced pressure to get 4-(1-(tetrahydro-2H-pyran-4-yl)-1H-pyrazol-5-yl)piperidine-4-carbonitrile hydrochloride (3.70 g, crude, HCl) as a yellow solid. LCMS: C₁₄H₂₀N₄O requires 260.2, found: m/z=261.2 (M+H)⁺.

### Step 6: Synthesis of 1-(6-chloropyridazin-4-yl)-4-(1-(tetrahydro-2H-pyran-4-yl)-1H-pyrazol-5-yl)piperidine-4-carbonitrile

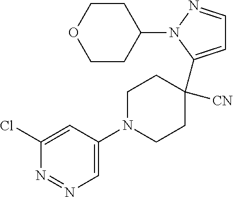 [SMILES: N#CC1(c2ccnn2C2CCOCC2)CCN(c2cnnc(Cl)c2)CC1]

To a solution of 4-(1-(tetrahydro-2H-pyran-4-yl)-1H-pyrazol-5-yl)piperidine-4-carbonitrile hydrochloride (3.70 g, 12.5 mmol, 1.00 eq, HCl) and 3,5-dichloropyridazine (1.86 g, 12.5 mmol, 1.00 eq) in IPA (20.0 mL) was added DIEA (6.44 g, 49.9 mmol, 8.69 mL, 4.00 eq) under N₂ atmosphere, and the mixture was stirred at 70-80° C. for 12 hrs. The reaction mixture was cooled to 25° C. and concentrated under reduced pressure. The crude was purified by pre-HPLC (column: Kromasil Eternity XT 250*80 mm*10 um; mobile phase: [H₂O (0.05% NH₃H₂O)-ACN]; gradient:10%-40% B over 20.0 min), concentrated to remove most ACN, then extracted with EtOAc (300 mL*3), the combined organic layer was washed with brine (500 mL), dried over Na₂SO₄, filtered and concentrated under reduced pressure to get 1-(6-chloropyridazin-4-yl)-4-(1-(tetrahydro-2H-pyran-4-yl)-1H-pyrazol-5-yl)piperidine-4-carbonitrile (3.00 g, 63.7% yield over three steps) as a brown solid. LCMS: C₁₁H₂₁ClN₆O requires 372.1, found: m/z=373.1 (M+H)⁺. ¹H NMR: (400 MHz, DMSO) δ 9.03 (d, J=2.0 Hz, 1H), 7.52 (s, 1H), 7.18 (d, J=1.6 Hz, 1H), 6.25 (s, 1H), 4.75-4.64 (m, 1H), 4.25 (br d, J=14.4 Hz, 2H), 3.97 (br dd, J=3.6, 11.6 Hz, 2H), 3.55 (br t, J=11.6 Hz, 2H), 3.28 (br t, J=13.2 Hz, 2H), 2.45 (br s, 2H), 2.15 (dq, J=4.4, 12.0 Hz, 2H), 2.08-1.99 (m, 2H), 1.80 (br d, J=10.4 Hz, 2H)

### Step 7: Synthesis of 1-(6-(2-(methoxymethoxy)phenyl)pyridazin-4-yl)-4-(1-(tetrahydro-2H-pyran-4-yl)-1H-pyrazol-5-yl)piperidine-4-carbonitrile

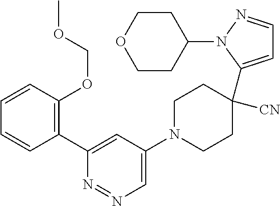 [SMILES: COCOc1ccccc1-c1cc(N2CCC(C#N)(c3ccnn3C3CCOCC3)CC2)cnn1]

To a solution of 1-(6-chloropyridazin-4-yl)-4-(1-(tetrahydro-2H-pyran-4-yl)-1H-pyrazol-5-yl)piperidine-4-carbonitrile (3.00 g, 8.05 mmol, 1.00 eq), (2-(methoxymethoxy)phenyl)boronic acid (2.93 g, 16.1 mmol, 2.00 eq) and K₂CO₃ (3.34 g, 24.1 mmol, 3.00 eq) in dioxane (30.0 mL) and H₂O (6.00 mL) was added Pd(dppf)Cl₂·CH₂Cl₂ (657 mg, 805 μmol, 0.100 eq) under N₂ atmosphere and the mixture was stirred at 110° C. for 12 hrs. The mixture was cooled to 25° C. and poured into H₂O (50.0 mL) extracted with EtOAc (30.0 mL*3), the combined organic layer was washed with brine (50.0 mL), dried over Na₂SO₄, filtered and concentrated under reduced pressure. The crude was purified by column chromatography (SiO₂, hexane/Ethyl acetate=1/1 to 0/1, petroleum ether/Ethyl acetate=0/1, R_{f}=0.1) to get 1-(6-(2-(methoxymethoxy)phenyl)pyridazin-4-yl)-4-(1-(tetrahydro-2H-pyran-4-yl)-1H-pyrazol-5-yl)piperidine-4-carbonitrile (2.50 g, 62.4% yield) as a light yellow solid. LCMS: C₂₆H₃₀N₆O₃ requires 474.2, found: m/z=475.2 (M+H). ¹H NMR: (400 MHz, DMSO) δ 9.04 (d, J=2.8 Hz, 1H), 7.63 (dd, J=1.6, 7.6 Hz, 1H), 7.52 (d, J=1.6 Hz, 1H), 7.46-7.39 (m, 1H), 7.31 (d, J=2.8 Hz, 1H), 7.24 (d, J=8.0 Hz, 1H), 7.13 (t, J=7.2 Hz, 1H), 6.27 (d, J=1.6 Hz, 1H), 5.21 (s, 2H), 4.74-4.63 (m, 1H), 4.22 (br d, J=14.0 Hz, 2H), 3.98 (br dd, J=4.0, 11.6 Hz, 2H), 3.55 (br t, J=11.6 Hz, 2H), 3.34-3.21 (m, 5H), 2.48 (br s, 2H), 2.22-2.00 (m, 4H), 1.81 (br d, J=10.4 Hz, 2H).

### Step 8: Synthesis of 1-(6-(2-(methoxymethoxy)phenyl)pyridazin-4-yl)-4-(1-(tetrahydro-2H-pyran-4-yl)-1H-pyrazol-5-yl)piperidine-4-carboxylic acid

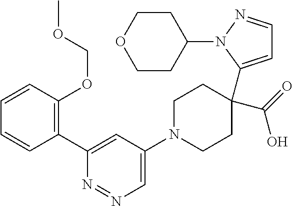 [SMILES: COCOc1ccccc1-c1cc(N2CCC(C(=O)O)(c3ccnn3C3CCOCC3)CC2)cnn1]

To a solution of 1-(6-(2-(methoxymethoxy)phenyl)pyridazin-4-yl)-4-(1-(tetrahydro-2H-pyran-4-yl)-1H-pyrazol-5-yl)piperidine-4-carbonitrile (2.50 g, 5.27 mmol, 1.00 eq) in THF (10.0 mL), MeOH (10.0 mL) and H₂O (10.0 mL) was added KOH (5.91 g, 105 mmol, 20.0 eq) at 25° C., then the mixture was stirred at 80° C. for 100 hrs. The mixture was concentrated in vacuum, adjusted pH to about 7 with 2N HCl (keep temperature below 25° C.), then concentrated under reduced pressure. The crude was purified by pre-HPLC (column: Kromasil Eternity XT 250*80 mm*10 um; mobile phase: [H₂O (0.05% NH₃H₂O)-ACN]; gradient:0%-25% B over 20.0 min) and lyophilization. 1-(6-(2-(methoxymethoxy)phenyl)pyridazin-4-yl)-4-(1-(tetrahydro-2H-pyran-4-yl)-1H-pyrazol-5-yl)piperidine-4-carboxylic acid (1.53 g, 57.9% yield) was obtained as a yellow solid. LCMS: C₂₆H₃₁N₅O₅ requires 493.2, found: m/z=494.3 (M+H)⁺. ¹H NMR: (400 MHz, DMSO) δ 9.04 (d, J=2.8 Hz, 1H), 7.63 (dd, J=1.6, 7.6 Hz, 1H), 7.52 (d, J=1.6 Hz, 1H), 7.46-7.39 (m, 1H), 7.31 (d, J=2.8 Hz, 1H), 7.24 (d, J=8.0 Hz, 1H), 7.13 (t, J=7.2 Hz, 1H), 6.27 (d, J=1.6 Hz, 1H), 5.21 (s, 2H), 4.74-4.63 (m, 1H), 4.22 (br d, J=14.0 Hz, 2H), 3.98 (br dd, J=4.0, 11.6 Hz, 2H), 3.55 (brt, J=11.6 Hz, 2H), 3.34-3.21 (m, 5H), 2.48 (br s, 2H), 2.22-2.00 (m, 4H), 1.81 (br d, J=10.4 Hz, 2H).

### Intermediate 22

### 1-(6-(2-(METHOXYMETHOXY)PHENYL)PYRIDAZIN-4-YL)-4-(1-METHYL-3-(TRIFLUOROMETHYL)-1H-PYRAZOL-5-YL)PIPERIDINE-4-CARBOXYLIC ACID

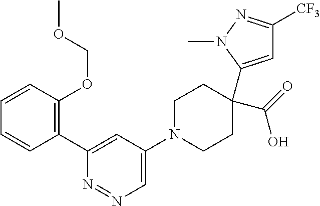 [SMILES: COCOc1ccccc1-c1cc(N2CCC(C(=O)O)(c3cc(C(F)(F)F)nn3C)CC2)cnn1]

The title compound was made as described for intermediate 21 beginning at step 3 and using 5-bromo-1-methyl-3-(trifluoromethyl)-1H-pyrazole to afford 1-(6-(2-(methoxymethoxy)phenyl)pyridazin-4-yl)-4-(1-methyl-3-(trifluoromethyl)-1H-pyrazol-5-yl)piperidine-4-carboxylic acid as a white solid. LCMS: C₂₃H₂₄F₃N₅O₄ requires 491.2, found: m/z=492.2 (M+H)⁺. ¹H NMR: (400 MHz, DMSO-d₆) δ 8.94 (br d, J=2.8 Hz, 1H), 7.62 (br d, J=7.6 Hz, 1H), 7.41 (brt, J=7.2 Hz, 1H), 7.26-7.17 (m, 2H), 7.11 (brt, J=7.6 Hz, 1H), 6.58 (s, 1H), 5.20 (s, 2H), 3.91 (s, 3H), 3.79 (br d, J=13.2 Hz, 2H), 3.40-3.34 (m, 2H), 3.31 (s, 3H), 2.42 (br d, J=13.2 Hz, 2H), 1.83 (brt, J=10.4 Hz, 2H).

### Intermediate 23

### 4-(1,4-DIMETHYL-1H-PYRAZOL-5-YL)-1-(6-(2-(METHOXYMETHOXY)PHENYL)PYRIDAZIN-4-YL)PIPERIDINE-4-CARBOXYLIC ACID

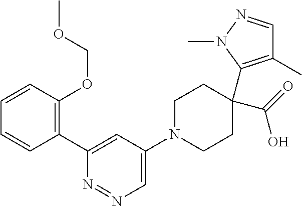 [SMILES: COCOc1ccccc1-c1cc(N2CCC(C(=O)O)(c3c(C)cnn3C)CC2)cnn1]

The title compound was made as described for intermediate 16 beginning at step 1 and using 2-(1,4-dimethyl-1H-pyrazol-5-yl)acetonitrile to afford 4-(1,4-dimethyl-1H-pyrazol-5-yl)-1-(6-(2-(methoxymethoxy)phenyl)pyridazin-4-yl)piperidine-4-carboxylic acid as a yellow solid. LCMS: C₂₃H₂₇N₅O₄ requires 437.2, found: m/z=438.2 (M+H)⁺. ¹H NMR: (400 MHz, DMSO) δ 8.92 (d, J=3.2 Hz, 1H), 7.62 (d, J=1.8, 7.6 Hz, 1H), 7.46-7.37 (m, 1H), 7.23 (dd, J=0.8, 8.4 Hz, 1H), 7.18 (d, J=3.2 Hz, 1H), 7.12 (d, J=1.2, 7.6 Hz, 1H), 6.98 (s, 1H), 5.21 (s, 2H), 3.88-3.81 (m, 5H), 3.32 (s, 3H), 3.32-3.24 (m, 2H), 2.48-2.44 (m, 1H), 2.07 (s, 3H), 1.89 (dt, J=4.0, 12.4 Hz, 2H)

### Intermediate 24

### 1-(6-(2-(METHOXYMETHOXY)PHENYL)PYRIDAZIN-4-YL)-4-(2-METHYL-2H-INDAZOL-3-YL)PIPERIDINE-4-CARBOXYLIC ACID

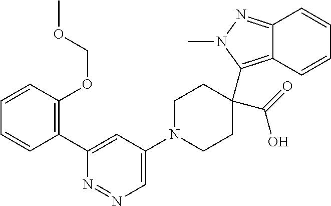 [SMILES: COCOc1ccccc1-c1cc(N2CCC(C(=O)O)(c3c4ccccc4nn3C)CC2)cnn1]

The title compound was made as described for intermediate 21 beginning at step 3 and using 3-bromo-2-methyl-2H-indazole to afford 1-(6-(2-(methoxymethoxy)phenyl)pyridazin-4-yl)-4-(2-methyl-2H-indazol-3-yl)piperidine-4-carboxylic acid as a white solid. LCMS: C₂₃H₂₇N₅O₄ requires 437.2, found: m/z=474.2 (M+H)⁺. ¹H NMR: (400 MHz, DMSO-d₆) δ 8.98 (d, J=3.2 Hz, 1H), 7.86 (d, J=8.8 Hz, 1H), 7.61 (dd, J=1.6, 7.6 Hz, 1H), 7.51 (d, J=8.7 Hz, 1H), 7.44-7.39 (m, 1H), 7.26-7.22 (m, 2H), 7.17 (dd, J=6.7, 8.6 Hz, 1H), 7.12 (dt, J=1.0, 7.5 Hz, 1H), 6.96 (ddd, J=0.8, 7.2, 8.2 Hz, 1H), 5.20 (s, 2H), 4.17 (s, 3H), 3.92 (br d, J=13.3 Hz, 2H), 3.39 (br s, 2H), 3.31 (s, 3H), 2.73 (br d, J=13.4 Hz, 2H), 2.42-2.33 (m, 2H).

### Intermediates 25-1 and 25-2

### (R)-3-METHOXY-1-(6-(2-(METHOXYMETHOXY)PHENYL)PYRIDAZIN-4-YL)PYRROLIDINE-3-CARBOXYLIC ACID (22-1) AND (S)-3-METHOXY-1-(6-(2-(METHOXYMETHOXY)PHENYL)PYRIDAZIN-4-YL)PYRROLIDINE-3-CARBOXYLIC ACID (22-2)

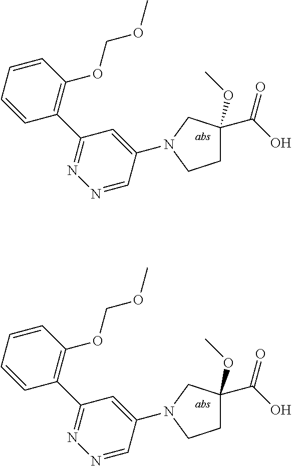 [SMILES: COCOc1ccccc1-c1cc(N2CC[C@](OC)(C(=O)O)C2)cnn1 ; COCOc1ccccc1-c1cc(N2CC[C@@](OC)(C(=O)O)C2)cnn1]

rac-(R)-3-methoxy-1-(6-(2-(methoxymethoxy)phenyl)pyridazin-4-yl)pyrrolidine-3-carboxylic acid (Intermediate 5) was subjected to SFC chromatography (Column: CELLULOSE-4 (30.0 mm*300 mmL; 5u) A: CO₂=75%, B: Cosolvent=0.1% AH in MeOH 25%). The separated isomers were collected and concentrated, affording Intermediate 25-1 (second-eluting isomer, RT=14.4 min, 950 mg, 45% yield) and Intermediate 25-2 (first-eluting isomer, RT=11.4 min, 950 mg, 45% yield) as white solids. Intermediate 25-1: LCMS C₁₈H₂₁N₃O₅ requires 359.2, found m/z=360.2 [M+H]⁺. Intermediate 25-2: LCMS C₁₈H₂₁N₃O₅ requires 359.2, found m/z=360.2 [M+H]⁺.

### Intermediate 26

### RAC-(R)-3-ETHOXY-1-(6-(2-(METHOXYMETHOXY)PHENYL)PYRIDAZIN-4-YL)PYRROLIDINE-3-CARBOXYLIC ACID, LITHIUM SALT

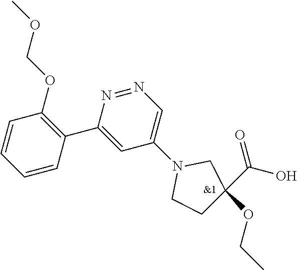 [SMILES: CCO[C@@]1(C(=O)O)CCN(c2cnnc(-c3ccccc3OCOC)c2)C1]

### Step 1: Synthesis of methyl 2-ethoxyacrylate

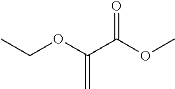

To a stirred solution of methyl 2-methoxyacrylate (3 g, 25.8 mmol) in EtOH (10 mL) were added CuCl₂ (195 mg, 1.45 mmol), NaH₂PO₄ (1.05 g, 8.76 mmol) and bis(benzonitrile)palladium(II) dichloride (496 mg, 1.29 mmol). The reaction was stirred at RT for 16 h. After completion of reaction (monitored by TLC), charcoal (2 g) was added and stirred at RT for 30 min. The reaction mixture was then filtered through a fritted funnel and washed with Et₂O (40 mL). The filtrate layer was concentrated under reduced pressure (below 40° C.). The crude material was diluted with water (20 mL) and extracted with Et₂O (2×50 mL). The combined organic layer was dried over anhydrous Na₂SO₄ and concentrated under reduced pressure (below 40° C.) to give the desired title compound as a colorless liquid (1.5 g, 45% yield). ¹H NMR (400 MHz, CDCl₃-d₆): δ 5.32 (d, J=2.0 Hz, 1H), 4.61 (d, J=2.0 Hz, 1H), 3.87-3.85 (m, 2H), 3.79 (s, 3H), 1.37 (t, J=7 Hz, 3H).

### Step 2: Synthesis of rac-methyl (R)-1-benzyl-3-ethoxypyrrolidine-3-carboxylate (4)

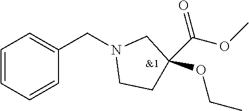 [SMILES: CCO[C@@]1(C(=O)OC)CCN(Cc2ccccc2)C1]

To a stirred solution of methyl 2-ethoxyacrylate (Step 1, 800 mg, 6.15 mmol) and N-benzyl-1-methoxy-N-((trimethylsilyl)methyl)methanamine (1.89 mL, 7.38 mmol) in DCM (10 mL) was added TFA (0.007 mL, 0.092 mmol) at 0° C. The resulting reaction mixture was stirred at RT for 16 h. After completion of reaction (monitored by TLC and LCMS). The reaction mixture was directly concentrated under reduced pressure. The crude was purified by flash column chromatography (5% EtOAc in heptane) to get the desired title compound as a colorless sticky gum (450 mg, 27% yield). LCMS C₁₅H₂₁NO₃ requires 263.2, found m/z=264.40 [M+H]⁺.

### Step 3: Synthesis of rac-methyl (R)-3-ethoxypyrrolidine-3-carboxylate

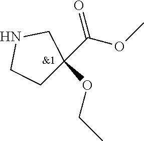

To a stirred solution of rac-methyl (R)-1-benzyl-3-ethoxypyrrolidine-3-carboxylate (450 mg, 1.71 mmol) in EtOH (10 mL) was added 10% Pd/C (225 mg, 50% w/w). The reaction mixture was degassed two times under H₂ atmosphere and stirred under hydrogen atmosphere for 16 h. After completion of the reaction (monitored by TLC and LCMS), the reaction mixture was filtered through a celite bed and washed with EtOH (50 mL). The filtrate was concentrated under reduced pressure to afford the title compound as a colorless viscous liquid (290 mg, 98% yield). LCMS C₈H₁₅NO₃ requires 173.1, found m/z=174.11 [M+H]⁺.

### Step 4: Synthesis of rac-methyl (R)-3-ethoxy-1-(6-(2-(methoxymethoxy)phenyl)pyridazin-4-yl)pyrrolidine-3-carboxylate

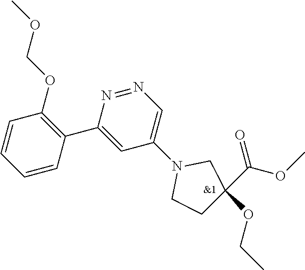 [SMILES: CCO[C@@]1(C(=O)OC)CCN(c2cnnc(-c3ccccc3OCOC)c2)C1]

To a stirred solution of methyl rac-methyl (R)-3-ethoxypyrrolidine-3-carboxylate (290 mg, 1.67 mmol) in DMSO (3 mL) was added DIPEA (0.877 mL, 5.02 mmol) and 5-chloro-3-(2-(methoxymethoxy)phenyl)pyridazine (630 mg, 2.51 mmol), the reaction mixture was stirred at 90° C. for 16 h. After completion of reaction (monitored by LCMS), the reaction mixture was diluted with water (50 mL) and extracted with EtOAc (2×100 mL). The combined organic layer was dried over anhydrous Na₂SO₄, filtered and concentrated under reduced pressure. The crude was purified by flash column chromatography (3% MeOH in DCM) to afford the title compound as a yellow sticky gum (300 mg, 46%). LCMS C₂₀H₂₅N₃O₅ requires 387.2, found m/z=388.4 [M+H]⁺.

### Step 5: Synthesis of the Title Compound (Intermediate 26)

To a stirred solution of rac-methyl (R)-3-ethoxy-1-(6-(2-(methoxymethoxy)phenyl)pyridazin-4-yl)pyrrolidine-3-carboxylate (300 mg, 0.774 mmol) in THF (10 mL) was added solution of lithium hydroxide (18.5 mg, 0.774 mmol) in water (5 mL). The resultant reaction mixture was stirred at RT for 16 h. After completion of reaction (monitored by TLC). Reaction mixture was concentrated under reduced pressure, diluted with 25% IPA in CHCl₃ (50 mL) and filtered through sintered funnel. Filtrate was concentrated under reduced pressure to afford the title compound as an off-white solid (263 mg, 91%).

LCMS C₁₉H₂₃N₃O₅ requires 373.2, found m/z=374.2 [M+H]⁺.

### Intermediate 27

### RAC-(R)-1-(6-(5-FLUORO-2-(METHOXYMETHOXY)PHENYL)PYRIDAZIN-4-YL)-3-METHOXYPYRROLIDINE-3-CARBOXYLIC ACID

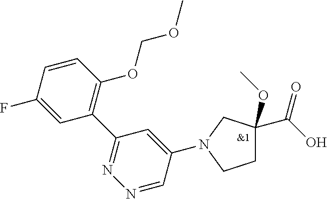 [SMILES: COCOc1ccc(F)cc1-c1cc(N2CC[C@@](OC)(C(=O)O)C2)cnn1]

### Step 1: Synthesis of rac-methyl (R)-1-(6-(5-fluoro-2-(methoxymethoxy)phenyl)pyridazin-4-yl)-3-methoxypyrrolidine-3-carboxylate

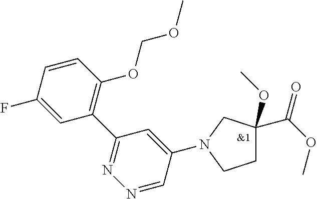 [SMILES: COCOc1ccc(F)cc1-c1cc(N2CC[C@@](OC)(C(=O)OC)C2)cnn1]

The title compound was prepared as described for Intermediate 1, Step 4, using rac-methyl (R)-1-(6-chloropyridazin-4-yl)-3-methoxypyrrolidine-3-carboxylate (Intermediate 5, Step 1) and (5-fluoro-2-(methoxymethoxy)phenyl)boronic acid to afford the title compound (407.3 mg, 52% yield) as a yellow foam. LCMS C₁₉H₂₂FN₃O₅ requires 391.2, found m/z=392.2 [M+H]⁺.

### Step 2: Synthesis of Title Compound (Intermediate 27)

The title compound was prepared as described for Intermediate 1, Step 5, using rac-methyl (R)-1-(6-(5-fluoro-2-(methoxymethoxy)phenyl)pyridazin-4-yl)-3-methoxypyrrolidine-3-carboxylate to afford the title compound. An additional acidification step to pH 7 with 3M HCl was performed followed by evaporation to dryness to isolate the title compound as the free acid in quantitative yield as a yellow oil. LCMS C₁₈H₂₀FN₃O₅ requires 377.2, found m/z=378.2. [M+H]⁺.

### Intermediate 28

### RAC-(R)-3-FLUORO-1-(6-(2-(METHOXYMETHOXY)PHENYL)PYRIDAZIN-4-YL)PYRROLIDINE-3-CARBOXYLIC ACID

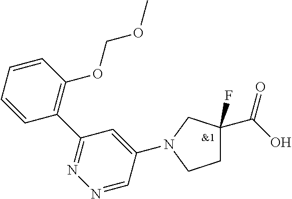 [SMILES: COCOc1ccccc1-c1cc(N2CC[C@@](F)(C(=O)O)C2)cnn1]

### Step 1: Synthesis of rac-methyl (R)-1-(6-chloropyridazin-4-yl)-3-fluoropyrrolidine-3-carboxylate

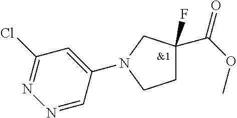 [SMILES: COC(=O)[C@]1(F)CCN(c2cnnc(Cl)c2)C1]

The title compound was synthesized as described for Intermediate 1, Step 3, using rac-methyl (R)-3-fluoropyrrolidine-3-carboxylate to afford the title compound (1.4 g, 40% yield) as a brown oil. LCMS C₁₀H₁₁ClFN₃O₂ requires 259.1, found m/z=260.2. [M+H]⁺.

### Step 2: Synthesis of the Title Compound (Intermediate 28)

rac-methyl (R)-1-(6-chloropyridazin-4-yl)-3-fluoropyrrolidine-3-carboxylate (1.4 g, 5.39 mmol) and potassium carbonate (5.39 mL, 2.2 g, 16.2 mmol) were combined and dissolved in 1,4 dioxane (21.60 mL), followed by sparging with N₂ for 10 minutes. Next, tetrakis(triphenylphosphine)palladium(O) (623 mg, 0.54 mmol) was added and the reaction mixture was placed in a pre-heated plate at 100° C. and stirred at this temperature until complete as judged by LCMS. After that, the reaction mixture was cooled down to room temperature and filtered to remove the precipitate formed upon cooling, with 10% MeOH in EA rinsing. The collected organic layer was washed with water (50 ml) and brine (50 ml), dried with anhydrous Na₂SO₄, filtered and then concentrated to a crude oil. The crude residue was purified via flash chromatography (0-100% EtOAc/Hex, then 40% MeOH in DCM) to afford the title compound (484.7 mg, 26% yield), as a yellow oil. LCMS C₁₇H₁₈FN₃O₄ requires 347.1, found m/z=348.2 [M+H]⁺.

### Intermediate 29

### RAC-(R)-2-(1-(4-(2,6-DIOXOPIPERIDIN-3-YL)PHENYL)-4-HYDROXYPIPERIDIN-4-YL)ACETALDEHYDE

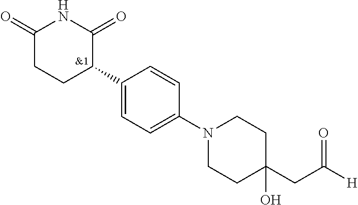 [SMILES: O=CCC1(O)CCN(c2ccc([C@@H]3CCC(=O)NC3=O)cc2)CC1]

### Step 1: Synthesis of 1-(4-nitrophenyl)piperidin-4-one

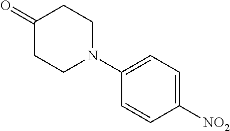 [SMILES: O=C1CCN(c2ccc([N+](=O)[O-])cc2)CC1]

To a solution of piperidin-4-one hydrochloride (100.0 g, 737.5 mmol) in DMF (500 mL) was added 4-Fluoronitrobenzene (83.3 g, 590.0 mmol) and DIPEA (286.0 g, 2212.5 mmol), then the reaction mixture was stirred at 80° C. for 5 h. The reaction mixture was then poured into water (1500 mL). The formed precipitate was collected by filtration, washed with water (800 mL) and dried to give a residue. The residue was triturated with Petroleum ether/EtOAc=5:1 (800 mL) and filtered to afford the title compound (110.0 g, 68% yield) as a yellow solid. LCMS C₁₁H₁₂N₂O requires 220.08, found m/z=221.1 [M+H]⁺.

### Step 2: Synthesis of tert-butyl 2-(4-hydroxy-1-(4-nitrophenyl)piperidin-4-yl)acetate

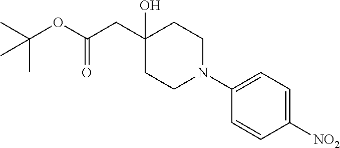 [SMILES: CC(C)(C)OC(=O)CC1(O)CCN(c2ccc([N+](=O)[O-])cc2)CC1]

To a solution of tert-butyl acetate (53.8 g, 463.2 mmol) in THF (640 mL) was added LDA (2M in THF) (386 mL, 771.9 mmol) dropwise at −78° C. And the reaction mixture was stirred at −78° C. for 30 min, then the mixture was added 1-(4-nitrophenyl)piperidin-4-one (Intermediate 30 Step 1, 85.0 g, 386.0 mmol) in THF (1280 mL) dropwise at −78° C., and the reaction mixture was stirred at −10° C. for 2 h. The reaction mixture was then quenched by the addition of saturated aqueous NH₄Cl solution (350 mL) and extracted with EtOAc (1000 mL×3). The combined organic phases were washed with brine (800 mL), dried over Na₂SO₄ and concentrated to afford the title compound (120.0 g, crude) as a brown solid, which was used for next step without any further purification. LCMS C₁₇H₂₄N₂O₅ requires 336.2, found m/z=337.2 [M+H]⁺.

### Step 3: Synthesis of tert-butyl 2-(1-(4-aminophenyl)-4-hydroxypiperidin-4-yl)acetate

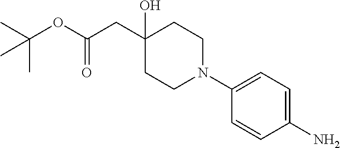 [SMILES: CC(C)(C)OC(=O)CC1(O)CCN(c2ccc(N)cc2)CC1]

To a solution of (tert-butyl 2-(4-hydroxy-1-(4-nitrophenyl)piperidin-4-yl)acetate (Intermediate 29 Step 2, 105.0 g, 312.1 mmol) in EtOH (2000 mL) and H₂O (400 mL) was added Fe powder (34.9 g, 624.0 mmol) and NH₄Cl (83.5 g, 1560.7 mmol) at room temperature. The mixture was stirred at 100° C. for 1 h. The mixture was then cooled to room temperature and filtered with EtOH rinsing, and the recovered filtrate was concentrated under reduced pressure to afford a crude residue. The residue was then dissolved in EtOAc (2000 mL) and washed with brine (500 mL), dried over Na₂SO₄ and filtered. The filtrate was concentrated in vacuo to give a residue. The residue was purified by silica gel column chromatography (Petroleum ether/EtOAc=2:1~1:1) to afford the title compound (70.0 g, 68% yield over two steps) as a yellow solid. LCMS C₁₇H₂₆N₂O₃ requires 306.2, found m/z=307.1 [M+H]⁺.

### Step 4: Synthesis of tert-butyl 2-(4-hydroxy-1-(4-iodophenyl)piperidin-4-yl)acetate

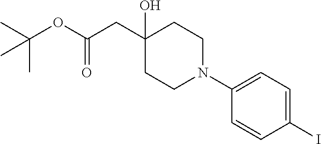 [SMILES: CC(C)(C)OC(=O)CC1(O)CCN(c2ccc(I)cc2)CC1]

To a solution of p-Toluenesulfonic acid monohydrate (117.4 g, 617.3 mmol) in t-BuOH (700 mL) was added tert-butyl 2-(1-(4-aminophenyl)-4-hydroxypiperidin-4-yl)acetate (Intermediate 30 Step 3, 70.0 g, 228.6 mmol) in ACN (350 mL) dropwise at 25° C. Then to the reaction mixture was added NaNO₂ (31.5 g, 456.9 mmol) and KI (94.9 g, 571.5 mmol) in H₂O (350 mL) dropwise at 25° C., and the reaction mixture was stirred at 60° C. for 4 h. To the mixture was added H₂O (5000 mL) and extracted with EtOAc (800 mL×3), the combined organic layers were washed with brine (500 mL), dried over Na₂SO₄ and filtered. The filtrate was concentrated in vacuo to give a residue. The residue was purified by silica gel column chromatography (Petroleum ether/EtOAc=10:1~5:1) to afford the title compound (54.2 g, 57% yield) as a yellow solid. LCMS C₁₇H₂₄INO₃ requires 417.1, found m/z=418.0 [M+H]⁺.

### Step 5: Synthesis of tert-butyl 2-(1-(4-(2,6-bis(benzyloxy)pyridin-3-yl)phenyl)-4-hydroxypiperidin-4-yl)acetate

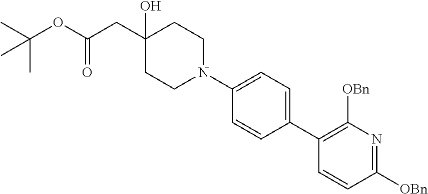 [SMILES: CC(C)(C)OC(=O)CC1(O)CCN(c2ccc(-c3ccc(OCc4ccccc4)nc3OCc3ccccc3)cc2)CC1]

To a solution of tert-butyl 2-(4-hydroxy-1-(4-iodophenyl)piperidin-4-yl)acetate (Intermediate 29, Step 4, 50.0 g, 119.8 mmol) in 1,2-dioxane (250 mL) and H₂O (50 mL) was added 2,6-bis(benzyloxy)-3-(4,4,5,5-tetramethyl-1,3,2-dioxaborolan-2-yl) pyridine (62.5 g, 149.8 mmol), Cs₂CO₃ (78.2 g, 239.9 mmol, and Pd(dppf)Cl₂ (2.5 g, 3.8 mmol, at room temperature. The mixture was stirred at 100° C. for 3 h. The mixture was then cooled to room temperature and filtered, the filtrate was concentrated to give a residue, then the residue was diluted with EtOAc (900 mL). The combined organic layers were washed with brine (400 mL), dried over Na₂SO₄ and filtered. The filtrate was concentrated in vacuo to give a residue. The residue was purified by silica gel column chromatography (Petroleum ether/EtOAc=8:1~1:1) afford the title compound (58.0 g, 84% yield) as a yellow solid. LCMS C₃₆H₄₀N₂O₅ requires 580.3, found m/z=581.1 [M+H]⁺.

### Step 6: Synthesis of 1-(4-(2,6-bis(benzyloxy)pyridin-3-yl)phenyl)-4-(2-hydroxyethyl)piperidin-4-ol

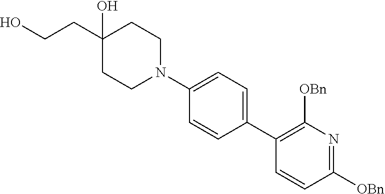 [SMILES: OCCC1(O)CCN(c2ccc(-c3ccc(OCc4ccccc4)nc3OCc3ccccc3)cc2)CC1]

To a solution of tert-butyl 2-(1-(4-(2,6-bis(benzyloxy)pyridin-3-yl)phenyl)-4-hydroxypiperidin-4-yl)acetate (Intermediate 29 Step 5, 30.0 g, 51.7 mmol) in THF (600 mL) was added LiAlH₄ (1M in THF) (103 mL, 103.3 mmol) dropwise at 0° under N₂, then the reaction mixture was stirred at room temperature for 12 h. Once complete, the reaction mixture was cooled to 0° C., and the reaction mixture was quenched via addition of H₂O (4 mL), followed by 15% aqueous NaOH (4 mL) dropwise. After quenching, the reaction was diluted with H₂O (12 mL), and the mixture was filtered through a celite pad. The filtrate was concentrated to dryness to give a residue. The residue was purified by silica gel column chromatography (Petroleum ether/EtOAc=1:1) to afford the title compound (21.0 g, 80% yield) as a white solid. LCMS C₃₂H₃₄N₂O₄ requires 510.3, found m/z=511.5 [M+H]⁺.

### Step 7: Synthesis of rac-(R)-3-(4-(4-hydroxy-4-(2-hydroxyethyl)piperidin-1-yl)phenyl)piperidine-2,6-dione

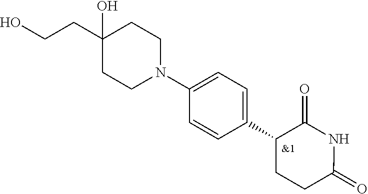 [SMILES: O=C1CC[C@H](c2ccc(N3CCC(O)(CCO)CC3)cc2)C(=O)N1]

A mixture of 1-(4-(2,6-bis(benzyloxy)pyridin-3-yl)phenyl)-4-(2-hydroxyethyl)piperidin-4-ol (Intermediate 29, Step 6, 19.0 g, 37.2 mmol) and Pd/C (7.6 g, 0.4 V) in DCM (285 mL) and MeOH (285 mL) was stirred under H₂ at room temperature for 12 h. Once complete, the mixture was filtered, and the filtrate was concentrated to afford the title compound (12.0 g, crude) as a white solid, which was used for next step without any further purification. LCMS C₁₈H₂₄N₂O₄ requires 332.2, found m/z=333.0 [M+H]⁺.

### Step 8: Synthesis of the Title Compound (Intermediate 29)

To a solution of rac-(R)-3-(4-(4-hydroxy-4-(2-hydroxyethyl)piperidin-1-yl)phenyl)piperidine-2,6-dione (Intermediate 29 Step 7, 10.5 g, 31.6 mmol) in DMSO (105 mL) was added IBX (17.9 g, 63.2 mmol), then the reaction mixture was stirred at room temperature for 2 h. The reaction mixture was added dropwise into saturated aqueous Na₂CO₃ solution (210 mL) at 0° C. and extracted with DCM (500 mL×4). The combined organic phases were washed with saturated aqueous Na₂S₂O₃ solution (300 mL×3), dried over Na₂SO₄ and concentrated to give a residue. The residue was triturated with EtOAc (40 mL) and filtered to afford the title compound (5.4 g, 50% yield over two steps) as a yellow solid. LCMS C₁₈H₂₂N₂O₄ requires 330.2, found m/z=331.0 [M+H]⁺.

### Intermediate 30

### RAC-(R)-3-(4-(2-oxo-7-AZASPIRO[3.5]NONAN-7-YL)PHENYL)PIPERIDINE-2,6-DIONE

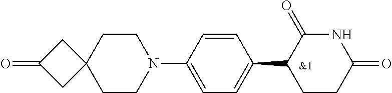

### Step 1: Synthesis of 2,6-bis(benzyloxy)-3-(4-bromophenyl)pyridine

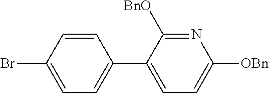 [SMILES: Brc1ccc(-c2ccc(OCc3ccccc3)nc2OCc2ccccc2)cc1]

To a mixture of 2,6-bis(benzyloxy)-3-(4,4,5,5-tetramethyl-1,3,2-dioxaborolan-2-yl)pyridine (6.00 g, 14.3 mmol), 1-bromo-4-iodobenzene (4.88 g, 17.2 mmol) in dioxane (60.0 mL) and H₂O (12.0 mL) was added K₂CO₃ (3.97 g, 28.7 mmol), Pd(dppf)Cl₂·CH₂Cl₂ (1.17 g, 1.44 mmol) at 15-25° C. under N₂. The resulting mixture was heated to 80° C. and stirred until complete as judged by LCMS. Once complete, mixture was poured into H₂O (150 mL), and extracted with ethyl acetate (50 mL×3). The combined organic layers were washed with brine (50.0 mL), dried with Na₂SO₄, filtered and concentrated in vacuo to a crude residue. The residue was purified by column chromatography (Petroleum ether/Ethyl acetate=70/1 to 10/1) to afford the title compound (4.70 g, 71% yield) as a white solid. LCMS C₂₅H₂₀BrNO₂ requires 445.1, found m/z=446.1 [M+H]⁺.

### Step 2: Synthesis of 7-(4-(2,6-bis(benzyloxy)pyridin-3-yl)phenyl)-7-azaspiro[3.5]nonan-2-one

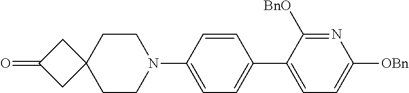 [SMILES: O=C1CC2(CCN(c3ccc(-c4ccc(OCc5ccccc5)nc4OCc4ccccc4)cc3)CC2)C1]

To a mixture of 2,6-bis(benzyloxy)-3-(4-bromophenyl)pyridine (4.30 g, 9.63 mmol), 7-azaspiro[3.5]nonan-2-one hydrochloride (2.20 g, 12.5 mmol) in 2-methyltetrahydrofuran (43.0 mL) was added Cs₂CO₃ (12.5 g, 38.5 mmol), DavePhos (758 mg, 1.93 mmol), Pd₂(dba)₃ (882 mg, 963 μmol) at 15-25° C. under N₂. The resulting mixture was stirred at 80° C. After 16 h, the mixture was cooled to 15-25° C., and more Pd₂(dba)₃ (1.32 g, 1.45 mmol), DavePhos (1.14 g, 2.89 mmol) was added under N₂. Then the resulting mixture was stirred at 80° C. until complete as judged by LCMS. Once complete, the reaction mixture was cooled to RT and the mixture was poured into H₂O (80.0 mL), and extracted with ethyl acetate (50 mL×3). The combined organic layers were washed with brine (50 mL), dried with Na₂SO₄, filtered and concentrated in vacuo to afford a crude residue. The residue was purified by column chromatography (SiO₂, Petroleum ether/Ethyl acetate=10/1 to 5/1). Then the purified residue was triturated with MeCN (15 mL) at 25° C. for 30 mins, then filtered with MeCN rinsing. The filtrate was concentrated under reduced pressure to afford a purified residue. The purified material was subjected to prep-HPLC (Phenomenex Luna C18 (250*70 mm, 10 um), H₂O/ACN with 0.1% FA, 70%-99%), and desired material was collected and concentrated under reduced pressure to remove MeCN. The remaining aqueous layer was adjusted pH to 7-8 with NaHCO₃ (solid), extracted with ethyl acetate (50.0 mL×3), and the combined organic layers were washed with brine (50.0 mL), dried with Na₂SO₄, filtered and concentrated in vacuum to afford the desired product (1.9 g, 38% yield) was obtained as a yellow solid. LCMS C₃₃H₃₂N₂O₃ requires 504.2, found m/z=505.2 [M+H]⁺.

### Step 3: Synthesis of the Title Compound (Intermediate 30)

To a mixture of 7-(4-(2,6-bis(benzyloxy)pyridin-3-yl)phenyl)-7-azaspiro[3.5]nonan-2-one (1.80 g, 3.57 mmol) in THF (45.0 mL) was added Pd/C (540 mg, 507 μmol, 10% purity), then Pd(OH)₂ (540 mg, 769 μmol, 20% purity) under N₂. The suspension was degassed and purged with H₂ 3 times. The mixture was stirred under H₂ (50 psi) at 25° C. until complete as judged by LCMS. The mixture was filtered through a pad of celite with cold THF rinsing and the combined filtrate was concentrated under reduced pressure to give a crude residue. The residue was purified by prep-HPLC (Waters Xbridge C18 150*50 mm*10 um, mobile phase: [water (NH4HCO3)-ACN]; gradient: 18%-48%) then lyophilized to afford the title compound (750 mg, 63% yield) as a white solid. LCMS C₁₉H₂₂N₂O₃ requires 326.2 found m/z=327.2 [M+H]⁺.

### Intermediate 31

### 4-(DIFLUOROMETHOXY)-1-(6-(2-(METHOXYMETHOXY)PHENYL)PYRIDAZIN-4-YL)PIPERIDINE-4-CARBOXYLIC ACID

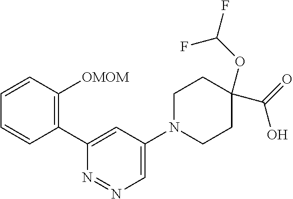

### Step 1: tert-butyl 4-((benzyloxy)methyl)-4-hydroxypiperidine-1-carboxylate

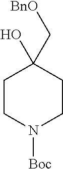 [SMILES: CC(C)(C)OC(=O)N1CCC(O)(COCc2ccccc2)CC1]

To a solution of BnOH (17.2 g, 159 mmol, 16.5 mL, 1.70 eq) in DMF (120 mL) was added NaH (7.50 g, 187 mmol, 60% purity, 2.00 eq) under N₂ at 0° C. and the reaction mixture was stirred at 25° C. for 0.5 hr. tert-butyl 1-oxa-6-azaspiro[2.5]octane-6-carboxylate (20.0 g, 93.7 mmol, 1.00 eq) in DMF (80.0 mL) was dropwise added to the above solution under N₂ at 0° C. and the reaction mixture was stirred at 25° C. for 2 hrs. The reaction mixture was quenched slowly by saturated NH₄Cl (150 mL) under N₂ at 0° C. The organic layers was extracted with ethyl acetate (150 mL*₂), washed with brine (200 mL), dried over Na₂SO₄ and concentrated. The residue was purified by column chromatography (SiO₂, Petroleum ether/Ethyl acetate=1/0~5/1, Petroleum ether/Ethyl acetate=3/1, R_{f}=0.30, PMA) and concentrated. Tert-butyl 4-((benzyloxy)methyl)-4-hydroxypiperidine-1-carboxylate (9.30 g, 24.6 mmol, 26.2% yield, 85.2% purity) was obtained as a yellow oil. LCMS: RT=0.548 min, m/z=322.3 (M+H)^{+ 1}H NMR (400 MHz, CDCl₃): δ 7.38-7.31 (m, 5H), 4.56 (s, 2H), 3.82 (s, 2H), 3.32 (s, 2H), 3.21-3.15 (m, 2H), 1.65-1.62 (m, 2H), 1.52-1.47 (m, 2H), 1.45 (s, 9H).

### Step 2: tert-butyl 4-((benzyloxy)methyl)-4-(difluoromethoxy)piperidine-1-carboxylate

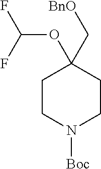 [SMILES: CC(C)(C)OC(=O)N1CCC(COCc2ccccc2)(OC(F)F)CC1]

To a solution of tert-butyl 4-((benzyloxy)methyl)-4-hydroxypiperidine-1-carboxylate (7.30 g, 22.7 mmol, 1.00 eq) and CuI (865 mg, 4.54 mmol, 0.20 eq) in ACN (70.0 mL) was dropwise added 2,2-difluoro-2-(fluorosulfonyl)acetic acid (16.1 g, 90.8 mmol, 9.39 mL, 4.00 eq) in ACN (20.0 mL) at 55° C. and the reaction mixture was stirred at 55° C. for 4 hrs. Additional 2,2-difluoro-2-(fluorosulfonyl)acetic acid (8.09 g, 45.4 mmol, 4.70 mL, 2.00 eq) was dropwise added to the above solution and the reaction mixture was stirred at 55° C. for 2 hrs. The reaction mixture was poured into saturated NaHCO₃ solution (200 mL) and extracted with EtOAc (150 mL*2). The organic layer was washed with brine (200 mL), dried over Na₂SO₄, filtered and concentrated. The residue was purified by column chromatography (SiO₂, Petroleum ether/Ethyl acetate=1/0~5/1, Petroleum ether/Ethyl acetate=3/1, R_{f}=0.43, PMA) and concentrated. The crude was purified by Prep-HPLC (column: Phenomenex luna C18 (250*70 mm, 10 um); mobile phase: [H₂O (0.05% HCl)-ACN]; gradient: 45%-85% B over 30.0 mins) and adjust to pH=8~9 with saturated NaHCO₃ solution. The organic layers were extracted with EtOAc (100 mL*2), washed with brine (150 mL), dried over Na₂SO₄, filtered and concentrated. Tert-butyl 4-((benzyloxy)methyl)-4-(difluoromethoxy)piperidine-1-carboxylate (1.10 g, 2.87 mmol, 12.6% yield, 96.8% purity) was obtained as yellow oil. LCMS: RT=0.631 min, m/z=272.1 (M-t-Bu+H)⁺. ¹H NMR (400 MHz, DMSO): δ 7.38-7.27 (m, 5H), 6.91 (t, J=75.6 Hz, 1H), 4.50 (s, 2H), 3.67-3.64 (m, 2H), 3.57 (s, 2H), 3.02 (s, 2H), 1.82-1.79 (m, 2H), 1.62-1.55 (m, 2H), 1.38 (s, 9H).

### Step 3: tert-butyl 4-((benzyloxy)methyl)-4-(difluoromethoxy)piperidine-1-carboxylate

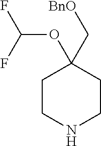 [SMILES: FC(F)OC1(COCc2ccccc2)CCNCC1]

To a solution of tert-butyl 4-((benzyloxy)methyl)-4-(difluoromethoxy)piperidine-1-carboxylate (1.10 g, 2.96 mmol, 1.00 eq) in DCM (10.0 mL) was added TFA (1.69 g, 14.8 mmol, 1.10 mL, 5.00 eq) and the reaction mixture was stirred at 25° C. for 3 hrs. The reaction mixture was adjusted to pH=8~9 with sat. NaHCO₃ solution and extracted with DCM (50.0 mL*2). The organic layer was washed with brine (80.0 mL), dried over Na₂SO₄, filtered and concentrated. Tert-butyl 4-((benzyloxy)methyl)-4-(difluoromethoxy)piperidine-1-carboxylate (800 mg, 2.95 mmol, 99.56% yield) was obtained as yellow oil. ¹H NMR (400 MHz, DMSO): δ 7.37-7.29 (m, 5H), 6.89 (t, J=75.6 Hz, 1H), 4.51 (s, 2H), 3.55 (s, 2H), 2.86-2.75 (m, 4H), 1.83-1.80 (m, 2H), 1.73-1.66 (m, 2H)

### Step 4: (4-(difluoromethoxy)piperidin-4-yl)methanol

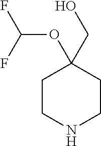 [SMILES: OCC1(OC(F)F)CCNCC1]

To a solution of Pd/C (0.50 g, 10% purity) in EtOH (50.0 mL) was added tert-butyl 4-((benzyloxy)methyl)-4-(difluoromethoxy)piperidine-1-carboxylate (1.00 g, 3.69 mmol, 1.00 eq) under N₂ atmosphere. The suspension was degassed and purged with H₂ for 3 times. The mixture was stirred under H₂ (50 Psi.) at 50° C. for 12 hrs. The reaction mixture was filtered and the filtrate was concentrated. (4-(difluoromethoxy)piperidin-4-yl)methanol (630 mg, 3.48 mmol, 94.3% yield) was obtained as yellow oil. ¹H NMR (400 MHz, DMSO): δ 6.91 (t, J=76.4 Hz, 1H), 3.49 (s, 2H), 2.85-2.77 (m, 4H), 1.74-1.64 (m, 4H).

### Step 5: (1-(6-chloropyridazin-4-yl)-4-(difluoromethoxy)piperidin-4-yl)methanol

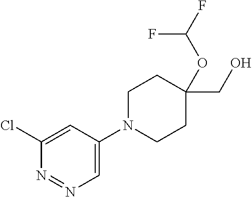 [SMILES: OCC1(OC(F)F)CCN(c2cnnc(Cl)c2)CC1]

To a solution of (4-(difluoromethoxy)piperidin-4-yl)methanol (630 mg, 3.48 mmol, 1.00 eq) in DMSO (6.00 mL) was added DIPEA (1.35 g, 10.4 mmol, 1.82 mL, 3.00 eq) and 3,5-dichloropyridazine (518 mg, 3.48 mmol, 1.00 eq) and the reaction mixture was stirred at 100° C. for 1 hr. The mixture was cooled to 25° C. The reaction mixture was poured into water (10.0 mL) and extracted with DCM (20.0 mL*2). The organic layer was washed with brine (20.0 mL), dried over Na₂SO₄, filtered and concentrated. The residue was purified by Prep-HPLC (column: Phenomenex luna C18 (250*70 mm, 10 um); mobile phase: [H₂O (0.05% HCl)-ACN]; gradient: 0%-40% B over 30.0 mins). The residue was concentrated and extracted with DCM (50.0 mL*2), washed with brine (70.0 mL), dried over Na₂SO₄, filtered and concentrated. (1-(6-chloropyridazin-4-yl)-4-(difluoromethoxy)piperidin-4-yl)methanol (640 mg, 1.53 mmol, 43.8% yield, 70% purity) was obtained as a brown solid. LCMS: RT=0.410 min, m/z=294.2 (M+H)⁺

### Step 6: (4-(difluoromethoxy)-1-(6-(2-(methoxymethoxy)phenyl)pyridazin-4-yl)piperidin-4-yl)methanol

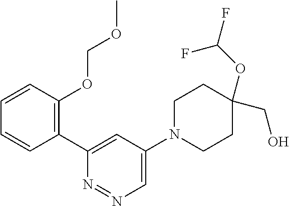 [SMILES: COCOc1ccccc1-c1cc(N2CCC(CO)(OC(F)F)CC2)cnn1]

To a solution of (1-(6-chloropyridazin-4-yl)-4-(difluoromethoxy)piperidin-4-yl)methanol (640 mg, 2.18 mmol, 1.00 eq) in dioxane (12.0 mL) and H₂O (3.00 mL) was added (2-(methoxymethoxy)phenyl)boronic acid (594 mg, 3.27 mmol, 1.50 eq), K₂CO₃ (602 mg, 4.36 mmol, 2.00 eq) and Pd(PPh₃)₄ (251 mg, 217 μmol, 0.10 eq) and the reaction mixture was stirred at 85° C. for 5 hrs. The mixture was poured into water (20.0 mL) and extracted with DCM (30.0 mL*2). The combined organic phase was washed with brine (20.0 mL), dried with anhydrous Na₂SO₄, filtered and concentrated in vacuum. The residue was purified by column chromatography (SiO₂, Petroleum ether/EtOAc=3/1~DCM/MeOH=5/1, DCM/MeOH=10/1, R_{f}=0.05, I₂) and concentrated in vacuum. (4-(difluoromethoxy)-1-(6-(2-(methoxymethoxy)phenyl)pyridazin-4-yl)piperidin-4-yl)methanol (700 mg, 1.50 mmol, 69.0% yield, 85% purity) was obtained as a yellow solid. LCMS: RT=0.493 min, m/z=396.3 (M+H)⁺. ¹H NMR (400 MHz, DMSO): δ 8.97 (s, 1H), 7.87-7.60 (m, 1H), 7.43-7.39 (m, 1H), 7.26-7.19 (m, 2H), 7.13-6.81 (m, 2H), 5.23-5.19 (m, 2H), 3.83-3.80 (m, 2H), 3.57-3.54 (m, 2H), 3.38-3.28 (m, 5H), 1.90-1.82 (m, 2H), 1.75-1.69 (m, 2H).

### Step 7: 4-(difluoromethoxy)-1-(6-(2-(methoxymethoxy)phenyl)pyridazin-4-yl)piperidine-4-carboxylic acid

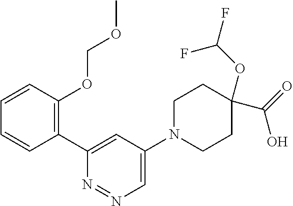 [SMILES: COCOc1ccccc1-c1cc(N2CCC(OC(F)F)(C(=O)O)CC2)cnn1]

To a solution of (4-(difluoromethoxy)-1-(6-(2-(methoxymethoxy)phenyl)pyridazin-4-yl)piperidin-4-yl)methanol (1.20 g, 3.03 mmol, 1.00 eq) in DCM (24.0 mL) was added DMP (5.15 g, 12.1 mmol, 3.76 mL, 4.00 eq) and the reaction mixture was stirred at 25° C. for 12 hrs. The reaction mixture was added sat.Na₂S₂O₃ solution (20.0 mL) and stirred for 1 hr, filtered and concentrated in vacuum. The crude was purified by Prep-HPLC (column: Kromasil Eternity XT 250*70 mm*10 um; mobile phase: [H₂O (10 mM NH₄HCO₃)-ACN]; gradient: 0%-20% B over 20.0 mins), filtered and concentrated in vacuum. The product was dissolved in water and freeze-dried directly. The crude was purified by Prep-HPLC (column: Kromasil Eternity XT 250*70 mm*10 um; mobile phase: [H₂O (0.05% NH₃H₂O)-ACN]; gradient: 0%-20% B over 20.0 mins). The product was dissolved in water and freeze-dried directly. 4-(difluoromethoxy)-1-(6-(2-(methoxymethoxy)phenyl)pyridazin-4-yl)piperidine-4-carboxylic acid (515 mg, 1.24 mmol, 40.7% yield, 98.1% purity) was obtained as a yellow solid. LCMS: RT=0.681 min, m/z=410.1 (M+H)⁺. ¹H NMR (400 MHz, DMSO): δ 8.98 (s, 1H), 7.63-7.61 (m, 1H), 7.43-7.39 (m, 1H), 7.26-7.22 (m, 2H), 7.14-6.77 (m, 2H), 5.20 (s, 2H), 3.88-3.85 (m, 2H), 3.31 (s, 3H), 3.29-3.23 (m, 2H), 2.11-2.03 (m, 2H), 1.90-1.86 (m, 2H)

### Intermediate 32

### 1-(6-(2-(METHOXYMETHOXY)PHENYL)PYRIDAZIN-4-YL)-4-METHYLPIPERIDINE-4-CARBOXYLIC ACID

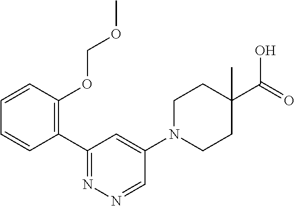 [SMILES: COCOc1ccccc1-c1cc(N2CCC(C)(C(=O)O)CC2)cnn1]

### Step 1: methyl 4-methylpiperidine-4-carboxylate hydrochloride

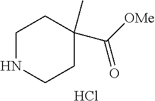

A mixture of 1-(tert-butyl) 4-methyl 4-methylpiperidine-1,4-dicarboxylate (1.00 g, 3.89 mmol, 1.00 eq) in HCl/EtOAc (15.0 mL, 2M) was stirred at 20° C. for 2 hrs. The mixture was concentrated to get methyl 4-methylpiperidine-4-carboxylate hydrochloride (750 mg, crude, HCl) as a light yellow solid. MS: m/z=158.2 (M+H)⁺

### Step 2: methyl 1-(6-chloropyridazin-4-yl)-4-methylpiperidine-4-carboxylate

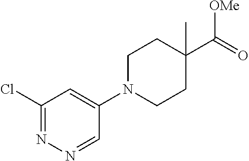 [SMILES: COC(=O)C1(C)CCN(c2cnnc(Cl)c2)CC1]

To a solution of methyl 4-methylpiperidine-4-carboxylate hydrochloride (550 mg, 2.84 mmol, 1.00 eq, HCl) and 3,5-dichloropyridazine (423 mg, 2.84 mmol, 1.00 eq) in IPA (5.00 mL) was added DIEA (1.47 g, 11.4 mmol, 1.98 mL, 4.00 eq) under N₂ atmosphere, and the mixture was stirred at 70° C. for 12 hrs. The mixture was diluted with MeOH (5.00 mL) and purified by prep-HPLC (column: Kromasil Eternity XT 250*70 mm*10 um; mobile phase: [H2O (0.05% NH3H2O)-ACN]; gradient:15%-45% B over 20.0 min), concentrated to remove most ACN, then extracted with EtOAc (100 mL*2), the combined organic layer was washed with brine (100 mL), dried over Na₂SO₄, filtered and concentrated. Methyl 1-(6-chloropyridazin-4-yl)-4-methylpiperidine-4-carboxylate (600 mg, 2.19 mmol, 77.0% yield over two steps, 98.3% purity) was obtained as a light yellow solid. LCMS: Rt=0.843 min, m/z=270.1 (M+H)⁺

### Step 3: methyl 1-(6-(2-(methoxymethoxy)phenyl)pyridazin-4-yl)-4-methylpiperidine-4-carboxylate

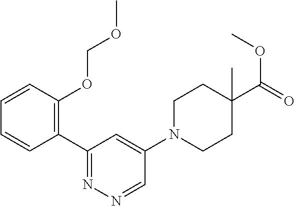 [SMILES: COCOc1ccccc1-c1cc(N2CCC(C)(C(=O)OC)CC2)cnn1]

To a solution of methyl 1-(6-chloropyridazin-4-yl)-4-methylpiperidine-4-carboxylate (600 mg, 2.22 mmol, 1.00 eq), (2-(methoxymethoxy)phenyl)boronic acid (810 mg, 4.45 mmol, 2.00 eq) and K₂CO₃ (922 mg, 6.67 mmol, 3.00 eq) in dioxane (6.00 mL) and H₂O (1.50 mL) was added Pd(dppf)Cl₂·CH₂Cl₂ (182 mg, 222 μmol, 0.100 eq) under N₂ atmosphere, and the mixture was stirred at 100° C. for 2 hrs. The mixture was cooled to 25° C. and poured into H₂O (20.0 mL), extracted with EtOAc (20.0 mL*3), the combined organic layer was washed with brine (30.0 mL), dried over Na₂SO₄, filtered and concentrated under reduced pressure. The residue was purified by column chromatography (SiO₂, Petroleum ether/Ethyl acetate=1/1 to 0/1, Petroleum ether/Ethyl acetate=0/1, R_{f}=0.2). methyl 1-(6-(2-(methoxymethoxy)phenyl)pyridazin-4-yl)-4-methylpiperidine-4-carboxylate (800 mg, 2.09 mmol, 94.0% yield, 97.1% purity) was obtained as a yellow oil. LCMS: Rt=0.499 min, m/z=372.2 (M+H)⁺

### Step 4: 1-(6-(2-(methoxymethoxy)phenyl)pyridazin-4-yl)-4-methylpiperidine-4-carboxylic acid

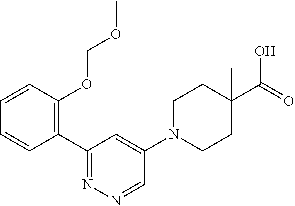 [SMILES: COCOc1ccccc1-c1cc(N2CCC(C)(C(=O)O)CC2)cnn1]

To a solution of methyl 1-(6-(2-(methoxymethoxy)phenyl)pyridazin-4-yl)-4-methylpiperidine-4-carboxylate (800 mg, 2.15 mmol, 1.00 eq) in MeOH (8.00 mL) and H₂O (4.00 mL) was added LiOH·H₂O (181 mg, 4.31 mmol, 2.00 eq), then the mixture was stirred at 60° C. for 12 hrs. The mixture was cooled to 30° C. and concentrated, adjusted pH to about 7 with 2N HCl (keep temperature below 25° C.), then concentrated under reduced pressure. The crude was purified by pre-HPLC (column: Kromasil Eternity XT 250*70 mm*10 um; mobile phase: [H2O (0.05% NH3H2O)-ACN]; gradient:0%-20% B over 20.0 min), concentrated to remove most ACN, then lyophilized. 1-(6-(2-(methoxymethoxy)phenyl)pyridazin-4-yl)-4-methylpiperidine-4-carboxylic acid (0.72 g, 2.00 mmol, 92.9% yield, 99.3% purity) was obtained as an off-white solid. LCMS: Rt=0.327 min, m/z=358.2 (M+H)⁺. ¹H NMR (400 MHz, DMSO): δ 8.88 (d, J=3.2 Hz, 1H), 7.61 (dd, J=1.6, 7.6 Hz, 1H), 7.44-7.35 (m, 1H), 7.21 (d, J=8.0 Hz, 1H), 7.17-7.07 (m, 2H), 5.20 (s, 2H), 3.72 (br d, J=13.2 Hz, 2H), 3.32 (s, 3H), 3.13 (br t, J=10.8 Hz, 2H), 2.06 (br d, J=13.2 Hz, 2H), 1.29-1.15 (m, 2H), 1.01 (s, 3H)

### Intermediate 33

### 1-(6-(2-(METHOXYMETHOXY)PHENYL)PYRIDAZIN-4-YL)-4-PROPYLPIPERIDINE-4-CARBOXYLIC ACID

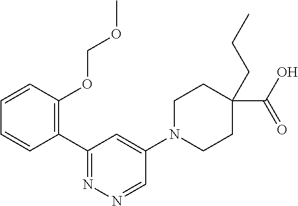 [SMILES: CCCC1(C(=O)O)CCN(c2cnnc(-c3ccccc3OCOC)c2)CC1]

### Step 1: 1-(tert-butyl) 4-methyl 4-propylpiperidine-1,4-dicarboxylate

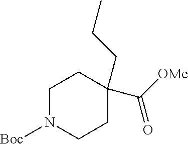 [SMILES: CCCC1(C(=O)OC)CCN(C(=O)OC(C)(C)C)CC1]

To a solution of 1-(tert-butyl) 4-methyl piperidine-1,4-dicarboxylate (4.00 g, 16.4 mmol, 1.00 eq) in THF (60.0 mL) was added LDA (2 M, 9.86 mL, 1.20 eq) at −65° C. under N₂ and stirred at −65° C. for 1 hr. Then 1-iodopropane (4.19 g, 24.6 mmol, 2.41 mL, 1.50 eq) in THF (20.0 mL) was added dropwise at −65° C. under N₂. The mixture was stirred at 25° C. for 12 hrs under N₂. The mixture was poured into saturated NH₄Cl (500 mL) aqueous solution slowly under N₂ at 0-10° C., extracted with EtOAc (200 mL*2). The combined organics were washed with brine (200 mL), dried over Na₂SO₄ and concentrated. The residue was used for next step without further purification. 1-(tert-butyl) 4-methyl 4-propylpiperidine-1,4-dicarboxylate (4.10 g, crude) was obtained as a yellow oil. ¹H NMR (400 MHz, CDCl₃): δ 3.80 (d, J=8.8 Hz, 2H), 3.63 (s, 3H), 2.78 (s, 2H), 2.09-1.98 (m, 2H), 1.45-1.35 (m, 11H), 1.34-1.22 (m, 2H), 1.18-1.10 (m, 2H), 0.80 (t, J=7.2 Hz, 3H)

### Step 2: methyl 4-propylpiperidine-4-carboxylate hydrochloride

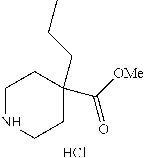

A solution of 1-(tert-butyl) 4-methyl 4-propylpiperidine-1,4-dicarboxylate (4.10 g, 14.3 mmol, 1.00 eq) in HCl/EtOAc (2 M, 100 mL) was stirred at 25° C. for 1 hr. The reaction mixture was concentrated under vacuum at 40° C. The residue was used for next step without further purification. Methyl 4-propylpiperidine-4-carboxylate hydrochloride (3.10 g, crude, HCl) was obtained as an off-white solid.

### Step 3: methyl 1-(6-chloropyridazin-4-yl)-4-propylpiperidine-4-carboxylate

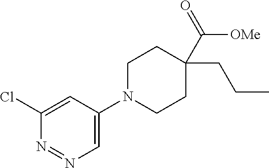 [SMILES: CCCC1(C(=O)OC)CCN(c2cnnc(Cl)c2)CC1]

To a solution of methyl 4-propylpiperidine-4-carboxylate hydrochloride (2.98 g, 13.4 mmol, 1.00 eq, HCl) and 3,5-dichloropyridazine (2.40 g, 16.1 mmol, 1.20 eq) in IPA (30.0 mL) was added DIEA (8.69 g, 67.2 mmol, 11.7 mL, 5.00 eq) under N₂ atmosphere, and the mixture was stirred at 70° C. for 12 hrs. The mixture was concentrated in vacuum at 40° C. The residue was purified by flash silica gel chromatography (SiO₂: Petroleum ether/Ethyl acetate=1/0 to 0/1, Commercial hexanes/Methanol=1/2, R_{f}=0.50, I₂). Methyl 1-(6-chloropyridazin-4-yl)-4-propylpiperidine-4-carboxylate (2.85 g, 9.55 mmol, 71.0% yield, 99.8% purity) was obtained as a brown solid. LCMS: RT=0.939 min, m/z=298.1 (M+H)⁺

¹HNMR (400 MHz, CDCl₃): δ 8.70 (d, J=2.8 Hz, 1H), 6.63 (d, J=2.8 Hz, 1H), 3.73 (s, 3H), 3.73-3.66 (m, 2H), 3.14-2.99 (m, 2H), 2.28 (d, J=13.4 Hz, 2H), 1.57-1.44 (m, 4H), 1.29-1.16 (m, 2H), 0.89 (t, J=7.2 Hz, 3H)

### Step 4: methyl 1-(6-(2-(methoxymethoxy)phenyl)pyridazin-4-yl)-4-propylpiperidine-4-carboxylate

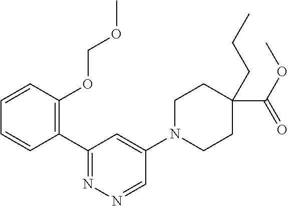 [SMILES: CCCC1(C(=O)OC)CCN(c2cnnc(-c3ccccc3OCOC)c2)CC1]

To a solution of methyl 1-(6-chloropyridazin-4-yl)-4-propylpiperidine-4-carboxylate (2.00 g, 6.72 mmol, 1.00 eq), (2-(methoxymethoxy)phenyl)boronic acid (2.44 g, 13.4 mmol, 2.00 eq) and K₂CO₃ (2.78 g, 20.1 mmol, 3.00 eq) in dioxane (20.0 mL) and H₂O (4.00 mL) was added Pd(dppf)Cl₂·CH₂Cl₂ (548 mg, 671 mol, 0.10 eq) at 25° C. under N₂ atmosphere, and the mixture was stirred at 100° C. for 12 hrs. The reaction mixture was cooled to 40° C. and diluted with water (200 mL) and extracted with EtOAc (100 mL*3), and the combined organic layers were washed with brine (200 mL), dried over Na₂SO₄ and concentrated to obtain crude product under vacuum. The residue was purified by column chromatography (SiO₂, Petroleum ether/Ethyl acetate=1/0 to 0/1, Dichloromethane/Methanol=0/1, R_{f}=0.30) to give methyl 1-(6-(2-(methoxymethoxy)phenyl)pyridazin-4-yl)-4-propylpiperidine-4-carboxylate (1.46 g, 3.60 mmol, 53.6% yield, 98.5% purity) was obtained as a black brown solid, which was confirmed by LCMS: RT=1.000 min, m/z=400.2 (M+H)⁺

### Step 5: 1-(6-(2-(methoxymethoxy)phenyl)pyridazin-4-yl)-4-propylpiperidine-4-carboxylic acid

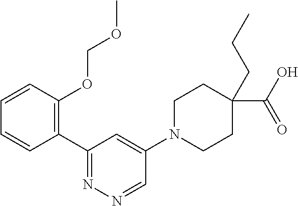 [SMILES: CCCC1(C(=O)O)CCN(c2cnnc(-c3ccccc3OCOC)c2)CC1]

To a solution of methyl 1-(6-(2-(methoxymethoxy)phenyl)pyridazin-4-yl)-4-propylpiperidine-4-carboxylate (1.00 g, 2.50 mmol, 1.00 eq) in MeOH (10.0 mL) was added a solution of LiOH·H₂O (315 mg, 7.51 mmol, 3.00 eq) in H₂O (5.00 mL). The mixture reaction was stirred at 60° C. for 12 hrs. The mixture was concentrated in vacuum, adjusted pH to about 7 with 2N HCl, concentrated in vacuum. The crude product was purified by reversed-phase HPLC (column: Kromasil Eternity XT 250*70 mm*10 um; mobile phase: [H2O (0.05% NH3H2O)-ACN]; gradient:0%-25% B over 20.0 min), concentrated to removed most ACN and lyophilization. 1-(6-(2-(methoxymethoxy)phenyl)pyridazin-4-yl)-4-propylpiperidine-4-carboxylic acid (0.70 g, 1.80 mmol, 71.8% yield, 99.0% purity) was obtained as a light yellow solid. LCMS: RT=0.696 min, m/z=386.2 (M+H)⁺. ¹H NMR (400 MHz, DMSO): δ 8.89 (d, J=2.8 Hz, 1H), 7.61 (d, J=1.6, 7.6 Hz, 1H), 7.44-7.36 (m, 1H), 7.22 (d, J=8.2 Hz, 1H), 7.18-7.07 (m, 2H), 5.20 (s, 2H), 3.80 (d, J=13.2 Hz, 2H), 3.32 (s, 3H), 3.06 (t, J=11.4 Hz, 2H), 2.08 (d, J=12.8 Hz, 2H), 1.38-1.17 (m, 6H), 0.80 (t, J=7.2 Hz, 3H)

### Intermediate 34

### 4-CYCLOPROPYL-1-(6-(2-(METHOXYMETHOXY)PHENYL)PYRIDAZIN-4-YL)PIPERIDINE-4-CARBOXYLIC ACID

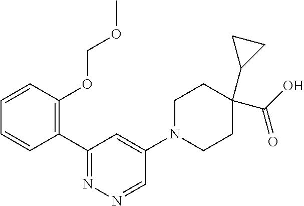 [SMILES: COCOc1ccccc1-c1cc(N2CCC(C(=O)O)(C3CC3)CC2)cnn1]

### Step 1: 1-(tert-butyl) 4-methyl 4-(1-hydroxyethyl)piperidine-1,4-dicarboxylate

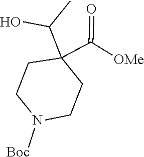 [SMILES: COC(=O)C1(C(C)O)CCN(C(=O)OC(C)(C)C)CC1]

To a solution of 1-(tert-butyl) 4-methyl piperidine-1,4-dicarboxylate (20.0 g, 82.2 mmol, 1.00 eq) in THF (200 mL) was added drop-wise LDA (2 M, 49.3 mL, 1.20 eq) at −65° C. under N₂ and stirred at −65° C. under N₂ for 1 hr. Then acetaldehyde (5.43 g, 123 mmol, 6.92 mL, 1.50 eq) in THF (40.0 mL) was added drop-wise at −65° C. under N₂. The mixture was warmed to 0° C. slowly and stirred at 0~25° C. for 2 hrs under N₂. The mixture was poured into saturated NH₄Cl (1.00 L) aqueous solution slowly under N₂ at 0~10° C., extracted with EtOAc (200 mL*2). The combined organics were washed with brine (500 mL), dried over Na₂SO₄ and concentrated. The crude was used for the next step directly. 1-(tert-butyl) 4-methyl 4-(1-hydroxyethyl)piperidine-1,4-dicarboxylate (23.6 g, crude) was obtained as a light yellow oil. ¹H NMR: (400 MHz, CDCl₃) δ 3.96 (s, 2H), 3.72 (s, 3H), 3.66-3.63 (m, 1H), 2.79-2.71 (m, 2H), 2.42 (d, J=7.2 Hz, 1H), 2.16-2.12 (m, 1H), 2.00-1.97 (m, 1H), 1.51-1.47 (m, 1H), 1.41 (s, 9H), 1.09 (d, J=6.8 Hz, 3H).

### Step 2: 1-(tert-butyl) 4-methyl 4-(1-(((trifluoromethyl)sulfonyl)oxy)ethyl)piperidine-1,4-dicarboxylate

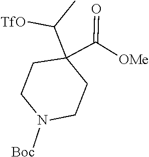 [SMILES: COC(=O)C1(C(C)OS(=O)(=O)C(F)(F)F)CCN(C(=O)OC(C)(C)C)CC1]

To a solution of 1-(tert-butyl) 4-methyl 4-(1-hydroxyethyl)piperidine-1,4-dicarboxylate (40.6 g, 141 mmol, 1.00 eq) in DCM (400 mL) was added dropwise Pyridine (44.7 g, 565 mmol, 45.6 mL, 4.00 eq) at 0° C. under N₂, and then Tf₂O (51.8 g, 184 mmol, 30.3 mL, 1.30 eq) was added dropwise at 0° C. under N₂. The resulting mixture was stirred at 25° C. for 12 hrs. The reaction mixture was used to the next step without work-up. 1-(tert-butyl) 4-methyl 4-(1-(((trifluoromethyl)sulfonyl)oxy)ethyl)piperidine-1,4-dicarboxylate (59.2 g, crude) was obtained as a black brown liquid.

### Step 3: 1-(tert-butyl) 4-methyl 4-vinylpiperidine-1,4-dicarboxylate

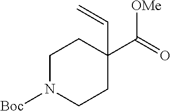 [SMILES: C=CC1(C(=O)OC)CCN(C(=O)OC(C)(C)C)CC1]

To a solution of 1-(tert-butyl) 4-methyl 4-(1-(((trifluoromethyl)sulfonyl)oxy)ethyl)piperidine-1,4-dicarboxylate (59.2 g, 141 mmol, 1.00 eq) in DCM (400 mL) was added DBU (64.5 g, 424 mmol, 63.9 mL, 3.00 eq) at 0° C. The mixture was stirred at 25° C. for 2 hrs. The reaction mixture was concentrated under reduced pressure to give a residue. The residue was purified by column chromatography (SiO₂, Commercial hexanes/Ethyl acetate=1/0 to 10/1; Rf=0.5, Commercial hexanes/Ethyl acetate=5:1, KMnO₄). Then the eluent was concentrated to give a residue. 1-(tert-butyl) 4-methyl 4-vinylpiperidine-1,4-dicarboxylate (2.11 g, 7.83 mmol, 5.54% yield) was obtained as a light yellow oil. ¹H NMR: (400 MHz, CDCl₃) δ 5.82-5.75 (m, 1H), 5.19-5.10 (m, 2H), 3.74-3.70 (m, 5H), 3.09-3.03 (m, 2H), 2.13 (d, J=13.6 Hz, 2H), 1.62-1.56 (m, 2H), 1.44 (s, 9H).

### Step 4: 1-(tert-butyl) 4-methyl 4-cyclopropylpiperidine-1,4-dicarboxylate

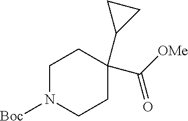 [SMILES: COC(=O)C1(C2CC2)CCN(C(=O)OC(C)(C)C)CC1]

Solution 1: 1-(tert-butyl) 4-methyl 4-vinylpiperidine-1,4-dicarboxylate, 1.00 eq, 2.11 g, 100%, 2.11 g in DCM, 42.0 mL

Solution 2: diazomethane, 6.00 eq, 1.9834 g, 0.75 M, 62.667 mL, 4.20%, 47.011 g (in 2-MeTHF)

Solution 3: Pd(OAc)₂, 0.01 eq, 0.0211 g, 0.01 M, 7.828 mL, 0.17%, 10.339 g (in DCM)

The solution 1 was pumped by Pump 1 S1, P1, 11.313 mL/min to flow reactor 1 FLR1, GL, CSTRs, 5.5 mL, 5° C.; flow reactor 2 FLR2, GL, CSTRs, 5.5 mL, 5° C.

The solution 2 was pumped by Pump 2 S2, P2, 16.187 mL/min to flow reactor 1 FLR1, GL, CSTRs, 5.5 mL, 5° C.; flow reactor 2 FLR2, GL, CSTRs, 5.5 mL, 5° C.

The solution 3 was pumped by Pump 3 S3, P3, 2.024 mL/min to flow reactor 2 FLR2, GL, CSTRs, 5.5 mL, 5° C.

The residence time of flow reactor 1 was FLR1, 0.2 min

The residence time of flow reactor 2 was FLR2, 0.186 min

The reaction mixture was filtered and the filtrate was concentrated under reduced pressure to give a residue. This crude was used directly in next step without further purification. 1-(tert-butyl) 4-methyl 4-cyclopropylpiperidine-1,4-dicarboxylate (1.55 g, crude) was obtained as a light yellow oil. ¹H NMR: δ 3.96 (s, 2H), 3.70 (s, 3H), 2.78-2.72 (m, 2H), 1.99 (d, J=12 Hz, 2H), 1.44 (s, 9H), 1.33-1.27 (m, 2H), 0.96-0.89 (m, 1H), 0.40-0.30 (m, 4H).

### Step 5: methyl 4-cyclopropylpiperidine-4-carboxylate hydrochloride

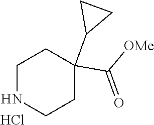 [SMILES: COC(=O)C1(C2CC2)CCNCC1 ; Cl]

A mixture of 1-(tert-butyl) 4-methyl 4-cyclopropylpiperidine-1,4-dicarboxylate (1.55 g, 5.47 mmol, 1.00 eq) and HCl/EtOAc (2 M, 50.0 mL) was stirred at 25° C. for 1 hr. The mixture was concentrated under reduced pressure to get a residue. The crude was used for the next step directly. Methyl 4-cyclopropylpiperidine-4-carboxylate hydrochloride (1.20 g, crude, HCl) was obtained as a yellow solid.

### Step 6: methyl 1-(6-chloropyridazin-4-yl)-4-cyclopropylpiperidine-4-carboxylate

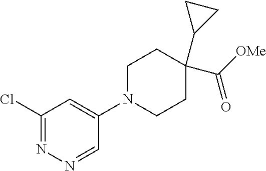 [SMILES: COC(=O)C1(C2CC2)CCN(c2cnnc(Cl)c2)CC1]

To a solution of methyl 4-cyclopropylpiperidine-4-carboxylate hydrochloride (1.20 g, 5.46 mmol, 1.00 eq, HCl) and 3,5-dichloropyridazine (813 mg, 5.46 mmol, 1.00 eq) in IPA (8.00 mL) was added DIEA (2.82 g, 21.8 mmol, 3.81 mL, 4.00 eq) under N₂ atmosphere, and the mixture was stirred at 70~80° C. for 12 hrs. The reaction mixture was cooled to 25° C. and concentrated under reduced pressure. The residue was purified by column chromatography (SiO₂, Commercial hexanes/Ethyl acetate=10/1 to 0/1; Rf=0.1, Commercial hexanes/Ethyl acetate=1/1). Then the eluent was concentrated under reduced pressure to give the product. Methyl 1-(6-chloropyridazin-4-yl)-4-cyclopropylpiperidine-4-carboxylate (1.33 g, 4.47 mmol, 81.8% yield, 99.4% purity) was obtained as a yellow solid. C₁₄H₁₈CN₃O₂ requires 295.1, found m/z=296.1 [M+H]⁺.

### Step 7: methyl 4-cyclopropyl-1-(6-(2-(methoxymethoxy)phenyl)pyridazin-4-yl)piperidine-4-carboxylate

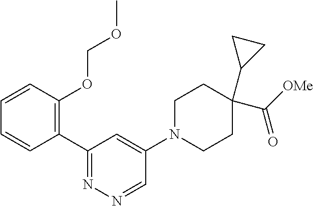 [SMILES: COCOc1ccccc1-c1cc(N2CCC(C(=O)OC)(C3CC3)CC2)cnn1]

To a solution of methyl 1-(6-chloropyridazin-4-yl)-4-cyclopropylpiperidine-4-carboxylate (1.33 g, 4.50 mmol, 1.00 eq), (2-(methoxymethoxy)phenyl)boronic acid (818 mg, 4.50 mmol, 1.00 eq) and K₂CO₃ (932 mg, 6.75 mmol, 1.50 eq) in dioxane (15.0 mL) and H₂O (3.00 mL) was added Pd(dppf)Cl₂·CH₂Cl₂ (184 mg, 225 μmol, 0.05 eq) under N₂ atmosphere and the mixture was stirred at 110° C. for 12 hrs. The mixture was cooled to 25° C. and concentrated under reduced pressure. The crude was purified by column chromatography (SiO₂, Hexane/Ethyl acetate=1/1 to 0/1, Petroleum ether/Ethyl acetate=0/1, Rf=0.1). Then the eluent was concentrated to get the product. Methyl 4-cyclopropyl-1-(6-(2-(methoxymethoxy)phenyl)pyridazin-4-yl)piperidine-4-carboxylate (1.30 g, 3.22 mmol, 71.5% yield, 98.3% purity) was obtained as a brown oil. LCMS C₂₂H₂₇N₃O₄ requires 397.2, found m/z=398.2 [M+H]⁺.

### Step 8: 4-cyclopropyl-1-(6-(2-(methoxymethoxy)phenyl)pyridazin-4-yl)piperidine-4-carboxylic acid

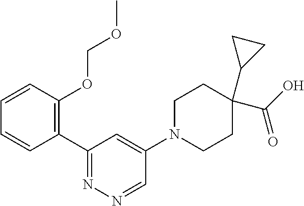 [SMILES: COCOc1ccccc1-c1cc(N2CCC(C(=O)O)(C3CC3)CC2)cnn1]

To a solution of methyl 4-cyclopropyl-1-(6-(2-(methoxymethoxy)phenyl)pyridazin-4-yl)piperidine-4-carboxylate (1.12 g, 2.82 mmol, 1.00 eq) in MeOH (10.0 mL) was added a solution of LiOH·H₂O (355 mg, 8.45 mmol, 3.00 eq) in H₂O (2.00 mL). The mixture was cooled to 25° C. and adjusted pH to about 7 with 1N HCl (keep temperature below 25° C.), then concentrated under reduced pressure. The crude was purified by pre-HPLC (basic condition: column: Kromasil Eternity XT 250*70 mm*10 um; mobile phase: [H₂O (0.05% NH₃H₂O)-ACN]; gradient: 0%-30% B over 20.0 min). Then the eluent was freeze-dried to get the product. 4-cyclopropyl-1-(6-(2-(methoxymethoxy)phenyl)pyridazin-4-yl)piperidine-4-carboxylic acid (510 mg, 1.30 mmol, 46.0% yield, 97.5% purity) was obtained as a yellow solid. LCMS: Rt=0.676 min, m/z=384.2 (M+H)⁺. ¹H NMR (400 MHz, DMSO): δ 8.91 (d, J=2.8 Hz, 1H), 7.62-7.60 (m, 1H), 7.42-7.38 (m, 1H), 7.22 (d, J=8.0 Hz, 1H), 7.17 (d, J=3.2 Hz, 1H), 7.13-7.09 (m, 1H), 5.20 (s, 2H), 3.93 (d, J=13.2 Hz, 2H), 3.32 (s, 3H), 2.95-2.89 (m, 2H), 1.99 (d, J=12.8 Hz, 2H), 1.33-1.26 (m, 2H), 0.93-0.86 (m, 1H), 0.33-0.24 (m, 4H).

### Intermediate 35

### 4-ETHYL-1-(6-(2-(METHOXYMETHOXY)PHENYL)PYRIDAZIN-4-YL)PIPERIDINE-4-CARBOXYLIC ACID

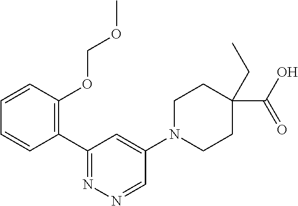 [SMILES: CCC1(C(=O)O)CCN(c2cnnc(-c3ccccc3OCOC)c2)CC1]

### Step 1: methyl 4-ethylpiperidine-4-carboxylate hydrochloride

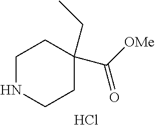

A mixture of 1-(tert-butyl) 4-methyl 4-ethylpiperidine-1,4-dicarboxylate (1.17 g, 4.31 mmol, 1.00 eq) in HCl/dioxane (2 M, 23.4 mL, 10.8 eq) was stirred at 25° C. for 12 hrs. The solvent was removed in vacuum. The crude methyl 4-ethylpiperidine-4-carboxylate hydrochloride (895 mg, 4.31 mmol, 99.9% yield, HCl) as a yellow solid was used into the next step without further purification.

### Step 2: methyl 1-(6-chloropyridazin-4-yl)-4-ethylpiperidine-4-carboxylate

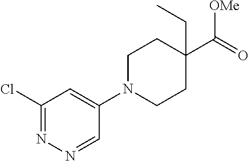 [SMILES: CCC1(C(=O)OC)CCN(c2cnnc(Cl)c2)CC1]

To a solution of methyl 4-ethylpiperidine-4-carboxylate hydrochloride (645 mg, 3.11 mmol, 1.00 eq, HCl) and 3,5-dichloropyridazine (462 mg, 3.11 mmol, 1.00 eq) in IPA (5.00 mL) was added DIEA (1.61 g, 12.4 mmol, 2.16 mL, 4.00 eq) under N₂ atmosphere, and the mixture was stirred at 70° C. for 5 hrs. The solvent was removed in vacuum. The residue was purified by prep-HPLC (column: Waters Xbridge C18 150*50 mm*10 um; mobile phase: [H2O (0.05% NH3H2O)-ACN]; gradient:23%-53% B over 12.0 min), the residue was extracted with DCM (50.0 mL*2), the combined organic layers were dried with Na₂SO₄, filtered and concentrated under vacuum. Compound methyl 1-(6-chloropyridazin-4-yl)-4-ethylpiperidine-4-carboxylate (735 mg, 2.59 mmol, 83.4% yield, 100% purity) was obtained as a light yellow solid. LCMS C₁₃H₁₈ClN₃O₂ requires 283.1, found m/z=284.1 [M+H]⁺.

### Step 3: methyl 4-ethyl-1-(6-(2-(methoxymethoxy)phenyl)pyridazin-4-yl)piperidine-4-carboxylate

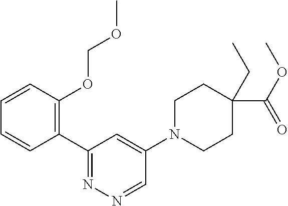 [SMILES: CCC1(C(=O)OC)CCN(c2cnnc(-c3ccccc3OCOC)c2)CC1]

To a solution of methyl 1-(6-chloropyridazin-4-yl)-4-ethylpiperidine-4-carboxylate (735 mg, 2.59 mmol, 1.00 eq), (2-(methoxymethoxy)phenyl)boronic acid (942 mg, 5.18 mmol, 2.00 eq), K₂CO₃ (1.07 g, 7.77 mmol, 3.00 eq) in dioxane (10.0 mL), H₂O (2.00 mL) was added Pd(dppf)Cl₂·CH₂Cl₂ (211 mg, 259 mol, 0.100 eq) under N₂ atmosphere, and the mixture was stirred at 100° C. for 12 hrs under N₂. The mixture was diluted with water (50.0 mL) and extracted with EtOAc (30.0 mL*2), and the combined organic layers were washed with brine (20.0 mL), dried over Na₂SO₄ and concentrated under vacuum. The residue was purified by column chromatography (SiO₂, Petroleum ether/Ethyl acetate=1/1 to 0/1, Petroleum ether/Ethyl acetate=0:1, R_{f}=0.08), concentrated in vacuum. Compound methyl 4-ethyl-1-(6-(2-(methoxymethoxy)phenyl)pyridazin-4-yl)piperidine-4-carboxylate (990 mg, 2.48 mmol, 95.68% yield, 96.5% purity) was obtained as a brown oil. LCMS C₂₁H₂₇N₃O₄ requires 385.2, found m/z=386.2 [M+H]⁺.

### Step 4: 4-ethyl-1-(6-(2-(methoxymethoxy)phenyl)pyridazin-4-yl)piperidine-4-carboxylic acid

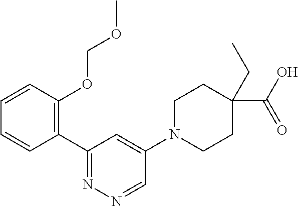 [SMILES: CCC1(C(=O)O)CCN(c2cnnc(-c3ccccc3OCOC)c2)CC1]

To a mixture of methyl 4-ethyl-1-(6-(2-(methoxymethoxy)phenyl)pyridazin-4-yl)piperidine-4-carboxylate (1.18 g, 3.06 mmol, 1.00 eq) in MeOH (5.00 mL) THF (5.00 mL) H₂O (1.50 mL) was added LiOH·H₂O (385 mg, 9.18 mmol, 3.00 eq) and then stirred at 60° C. for 12 hrs. The solvent was removed in vacuum, the residue was adjusted pH to about 6-7. The residue was purified by prep-HPLC (column: Kromasil Eternity XT 250*70 mm*10 um; mobile phase: [H2O (0.05% NH3H2O)-ACN]; gradient:0%-20% B over 20.0 min), lyophilized. Compound 4-ethyl-1-(6-(2-(methoxymethoxy)phenyl)pyridazin-4-yl)piperidine-4-carboxylic acid (0.88 g, 2.37 mmol, 77.3% yield, 99.9% purity) was obtained as a light yellow solid. LCMS C₂₀H₂₅N₃O₄ requires 371.2, found m/z=372.3 [M+H]⁺. ¹H NMR (400 MHz, DMSO): δ 8.92 (d, J=2.8 Hz, 1H), 7.62 (dd, J=1.2, 7.6 Hz, 1H), 7.43-7.37 (m, 1H), 7.22 (d, J=8.4 Hz, 1H), 7.18 (d, J=2.8 Hz, 1H), 7.11 (t, J=7.6 Hz, 1H), 5.20 (s, 2H), 3.84 (br d, J=13.2 Hz, 2H), 3.33 (s, 3H), 3.04 (br t, J=11.2 Hz, 2H), 2.06 (br d, J=13.2 Hz, 2H), 1.47 (q, J=7.2 Hz, 2H), 1.38-1.27 (m, 2H), 0.78 (t, J=7.2 Hz, 3H)

### Intermediate 36

### RAC-(R)-2-(1-(4-(2,6-DIOXOPIPERIDIN-3-YL)PHENYL)-4-METHOXYPIPERIDIN-4-YL)ACETALDEHYDE

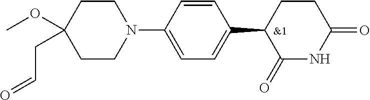 [SMILES: COC1(CC=O)CCN(c2ccc([C@H]3CCC(=O)NC3=O)cc2)CC1]

### Step 1: tert-butyl 4-(2-ethoxy-2-oxoethyl)-4-methoxypiperidine-1-carboxylate

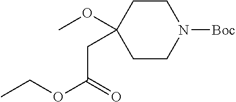 [SMILES: CCOC(=O)CC1(OC)CCN(C(=O)OC(C)(C)C)CC1]

To a solution of tert-butyl 4-(2-ethoxy-2-oxoethyl)-4-hydroxypiperidine-1-carboxylate (30.0 g, 104 mmol, 1.00 eq) in DMF (300 mL) was added Ag₂O (72.5 g, 313 mmol, 3.00 eq) at 20~25° C. under N₂. Then CH₃I (74.0 g, 522 mmol, 32.5 mL, 5.00 eq) was added into the mixture at 0~10° C. under N₂ and stirred at 0~10° C. under N₂ for 16 hrs. The mixture was poured into 20% AcOK solution (1.00 L) under N₂ atmosphere, the aqueous phase was extracted with MTBE (800 mL*2). The combined organic phase was washed with brine (500 mL), dried with anhydrous Na₂SO₄, filtered and concentrated in vacuum. The residue was purified by column chromatography (SiO₂, Petroleum ether/Ethyl acetate=100/1 to 10/1, Petroleum ether/Ethyl acetate=2/1, R_{f}=0.50). The eluent was concentrated under vacuum at 40° C. to get product. tert-butyl 4-(2-ethoxy-2-oxoethyl)-4-methoxypiperidine-1-carboxylate (25.1 g, 83.2 mmol, 79.7% yield) was obtained as a yellow oil. LCMS: RT=0.140 min, m/z=202.1 (M-BOC+H)^{+ 1}H NMR (400 MHz, CDCl₃): δ 4.15-4.10 (m, 2H), 3.68-3.66 (m, 2H), 3.24 (s, 3H), 3.06-3.04 (m, 2H), 2.47-2.44 (m, 2H), 1.86-1.83 (m, 2H), 1.59-1.53 (m, 2H), 1.43 (s, 9H), 1.28-1.23 (m, 3H)

### Step 2: tert-butyl 4-(2-hydroxyethyl)-4-methoxypiperidine-1-carboxylate

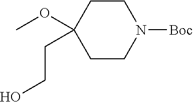 [SMILES: COC1(CCO)CCN(C(=O)OC(C)(C)C)CC1]

The solution of LAH (2.50 M, 33.3 mL, 1.00 eq) in THF (125 mL) was degassed and purged with N₂ for 3 times. Then the solution of tert-butyl 4-(2-ethoxy-2-oxoethyl)-4-methoxypiperidine-1-carboxylate (25.1 g, 83.2 mmol, 1.00 eq) in THF (125 mL) was drop-wise added into the mixture at 0° C. under N₂ flow and the reaction mixture was stirred at 0° C. under N₂ flow for 2 hrs. The reaction mixture was cool to −10~0° C., then dropwise added H₂O (3.16 mL) at 0° C. under N₂ and stirred for 0.5 hr. The mixture was dropwise added 15% NaOH solution (3.16 mL) and H₂O (9.48 mL) and stirred at 15~25° C. for 15 mins. Then Na₂SO₄ (30.0 g) was added into the mixture and stirred for 15 mins. The mixture was filtered and the cake was washed by THF (100 mL*2). The filtrate was combined and concentrated tert-butyl 4-(2-hydroxyethyl)-4-methoxypiperidine-1-carboxylate (21.6 g, crude) was obtained as a yellow oil. LCMS: RT=0.127 min, m/z=160.2 (M-BOC+H)^{+ 1}H NMR (400 MHz, CDCl₃): δ 3.74-3.69 (m, 4H), 3.17 (s, 3H), 3.09-3.04 (m, 2H), 2.65-2.63 (m, 1H), 1.83-1.70 (m, 4H), 1.44-1.42 (m, 2H), 1.41 (s, 9H)

### Step 3: 2-(4-methoxypiperidin-4-yl)ethan-1-ol hydrochloride

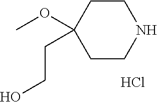

A solution of tert-butyl 4-(2-hydroxyethyl)-4-methoxypiperidine-1-carboxylate (21.6 g, 83.2 mmol, 1.00 eq) in HCl/dioxane (2.00 M, 216 mL, 5.19 eq) was stirred at 25° C. for 12 hrs. The reaction mixture was concentrated in vacuum at low temperature. 2-(4-methoxypiperidin-4-yl)ethan-1-ol hydrochloride (20.8 g, crude, HCl) was obtained as a yellow oil. LCMS: RT=0.110 min, m/z=160.2 (M+H)^{+ 1}H NMR: (400 MHz, DMSO-d₆): δ 9.21-9.08 (m, 2H), 3.44-3.40 (m, 2H), 3.06 (s, 3H), 3.01-2.98 (m, 2H), 2.87-2.78 (m, 2H), 1.85-1.82 (m, 2H), 1.74-1.61 (m, 4H)

### Step 4: 2-(1-(4-(2,6-bis(benzyloxy)pyridin-3-yl)phenyl)-4-methoxypiperidin-4-yl)ethan-1-ol

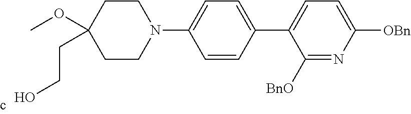 [SMILES: COC1(CCO)CCN(c2ccc(-c3ccc(OCc4ccccc4)nc3OCc3ccccc3)cc2)CC1]

To a solution of 2-(4-methoxypiperidin-4-yl)ethan-1-ol hydrochloride (31.5 g, 70.5 mmol, 1.00 eq), 2,6-bis(benzyloxy)-3-(4-bromophenyl)pyridine (20.7 g, 105 mmol, 1.50 eq, HCl) and Cs₂CO₃ (68.9 g, 211 mmol, 3.00 eq) in DMF (315 mL) was added Xphos Pd G4 (6.07 g, 7.06 mmol, 0.10 eq) at 25° C. under N₂. The reaction mixture was stirred at 80° C. for 12 hrs under N₂. The reaction mixture was cooled to 25° C. and poured into H₂O (1.00 L). The mixture was extracted with EtOAc (600 mL*3). The combined organic layer was washed with brine (500 mL*2), dried over Na₂SO₄ and filtered. The combined organic layer was concentrated under reduced pressure. The residue was purified by column chromatography (SiO₂, Petroleum ether: Ethyl acetate=2/1 to 1/3, Petroleum ether/Ethyl acetate=1/1, R_{f}=0.10). The eluent was concentrated under vacuum at 40° C. to yield 2-(1-(4-(2,6-bis(benzyloxy)pyridin-3-yl)phenyl)-4-methoxypiperidin-4-yl)ethan-1-ol (11.5 g, 18.0 mmol, 25.5% yield, 82.2% purity) as a yellow oil. LCMS: RT=0.622 min, m/z=525.3 (M+H)⁺

### Step 5: rac-(R)-3-(4-(4-(2-hydroxyethyl)-4-methoxypiperidin-1-yl)phenyl)piperidine-2,6-dione

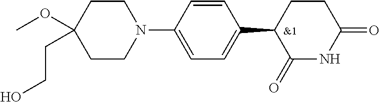 [SMILES: COC1(CCO)CCN(c2ccc([C@H]3CCC(=O)NC3=O)cc2)CC1]

A Parr bottle was purged with N₂, then added Pd/C (5.75 g, 5.40 mmol, 10% purity, 2.46^{e-1} eq) and Pd(OH)₂ (5.75 g, 20.4 mmol, 50% purity, 9.34^{e-1} eq) in the bottle under N₂, finally 2-(1-(4-(2,6-bis(benzyloxy)pyridin-3-yl)phenyl)-4-methoxypiperidin-4-yl)ethan-1-ol (11.5 g, 21.9 mmol, 1.00 eq) in THF (115 mL) and dioxane (115 mL) was added in the bottle. The suspension was degassed and purged with H₂ 3 times. The mixture was stirred under H₂ (50 psi) at 25° C. for 16 hrs. The mixture was filtered and washed with dioxane (500 mL). The filtrate was concentrated give the residue. The crude product was triturated with MTBE (100 mL) at 25° C. for 30 mins. The suspension was filtered and the filter cake was washed with MTBE (100 mL), dried under vacuum. rac-(R)-3-(4-(4-(2-hydroxyethyl)-4-methoxypiperidin-1-yl)phenyl)piperidine-2,6-dione (5.20 g, 13.3 mmol, 61.0% yield, 89.1% purity) was obtained as a gray solid.

LCMS: RT=0.407 min, m/z=347.1 (M+H)⁺

### Step 6: rac-(R)-2-(1-(4-(2,6-dioxopiperidin-3-yl)phenyl)-4-methoxypiperidin-4-yl)acetaldehyde

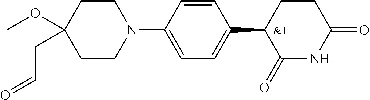 [SMILES: COC1(CC=O)CCN(c2ccc([C@H]3CCC(=O)NC3=O)cc2)CC1]

To a solution of rac-(R)-3-(4-(4-(2-hydroxyethyl)-4-methoxypiperidin-1-yl)phenyl)piperidine-2,6-dione (5.20 g, 15.0 mmol, 1.00 eq) in DMSO (52.0 mL) was added IBX (6.30 g, 22.5 mmol, 1.50 eq) at 20~30° C. under N₂. The mixture was stirred at 20-30° C. for 1 hr under N₂. The mixture was cooled to 15° C. and quenched with Na₂S₂O₃ (60.0 mL) under pH~5 (with 1 N citric acid aq.). The mixture was extracted by DCM (50.0 mL*5), the organic layer is washed by water (20.0 mL*1) and dry by Na₂SO₄. The residue was purified by prep-HPLC (column: Phenomenex luna Cis (250*70 mm, 10 um); mobile phase: [H₂O (0.05% HCl)-ACN]; gradient: 1%-20% B over 27.0 mins). The residue was adjusted to pH=7~8 with sat.NaHCO₃ solution and extracted with DCM (200 mL*2). The organic layers were washed with water (300 mL), dried over Na₂SO₄ and concentrated. rac-(R)-2-(1-(4-(2,6-dioxopiperidin-3-yl)phenyl)-4-methoxypiperidin-4-yl)acetaldehyde (1.70 g, 4.84 mmol, 32.2% yield, 98.0% purity) was obtained as a yellow solid. LCMS: RT=0.413 min, m/z=345.1 (M+H)⁺. ¹H NMR (400 MHz, DMSO-d₆): δ 10.77 (s, 1H), 9.75-9.74 (m, 1H), 7.05-7.02 (m, 2H), 6.09-6.88 (m, 2H), 3.73-3.69 (m, 1H), 3.35-3.34 (m, 1H), 3.33-3.32 (m, 1H), 3.18 (s, 3H), 3.00-2.93 (m, 2H), 2.64-2.59 (m, 3H), 2.49-2.43 (m, 1H), 2.17-2.07 (m, 1H), 2.03-1.99 (m, 1H), 1.97-1.90 (m, 2H), 1.76-1.69 (m, 2H).

### Intermediate 37

### RAC-(R)-2-(1-(4-(2,6-DIOXOPIPERIDIN-3-YL)PHENYL)-4-FLUOROPIPERIDIN-4-YL)ACETALDEHYDE

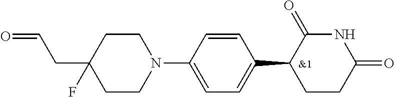 [SMILES: O=CCC1(F)CCN(c2ccc([C@@H]3CCC(=O)NC3=O)cc2)CC1]

### Step 1: tert-butyl 4-fluoro-4-(2-hydroxyethyl)piperidine-1-carboxylate

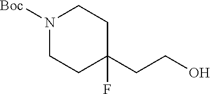 [SMILES: CC(C)(C)OC(=O)N1CCC(F)(CCO)CC1]

A mixture of tert-butyl 4-(2-ethoxy-2-oxoethyl)-4-fluoropiperidine-1-carboxylate (60.0 g, 207 mmol, 1.00 eq) in THF (600 mL) was degassed under vacuum and purged with N₂ 3 times. The mixture was cooled to −20~0° C., then LAH (2.50 M, 82.9 mL, 1.00 eq) was added to the mixture dropwise at −20~0° C. under N₂. The resulting mixture was stirred at −20~0° C. for 1 hr under N₂. The reaction mixture was quenched with water (8.00 mL) at 0° C. under N₂. The reaction mixture was added 8.00 mL NaOH (10%) solution dropwise, and diluted with 24.0 mL water at 0° C. under N₂. The reaction mixture was filtered to collect the filtrate. The filtrate was dried over Na₂SO₄, concentrated in vacuo. tert-butyl 4-fluoro-4-(2-hydroxyethyl)piperidine-1-carboxylate (51.2 g, 207 mmol, 99.8% yield) was obtained as yellow oil. MS: m/z=192.2 (M+H)⁺. ¹H NMR (400 MHz, DMSO-d₆): δ 4.47 (t, J=4.8 Hz, 1H), 3.75-3.72 (d, J=12.8 Hz, 2H), 3.55-3.51 (m, 2H), 2.96 (s, 2H), 1.82-1.50 (m, 6H), 1.39 (s, 9H)

### Step 2: 2-(4-fluoropiperidin-4-yl)ethan-1-ol hydrochloride

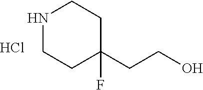

To a solution of tert-butyl 4-fluoro-4-(2-hydroxyethyl)piperidine-1-carboxylate (51.2 g, 207 mmol, 1.00 eq) in dioxane (50.0 mL) was added HCl/dioxane (2.00 M, 250 mL, 2.42 eq) and the reaction mixture was stirred for 12 hrs at 15-25° C. The reaction mixture was concentrated under reduced pressure. 2-(4-fluoropiperidin-4-yl)ethan-1-ol hydrochloride (35.0 g, 190 mmol, 92.0% yield, HCl) was obtained as a white solid. MS: m/z=148.2 (M+H)⁺. ¹H NMR: (400 MHz, DMSO-d₆): δ 9.10 (s, 2H), 4.72 (s, 1H), 3.56-3.52 (m, 2H), 3.18-3.15 (m, 2H), 2.95-2.93 (m, 2H), 2.01-1.90 (m, 4H), 1.87-1.76 (m, 2H).

### Step 3: 2-(1-(4-(2,6-bis(benzyloxy)pyridin-3-yl)phenyl)-4-fluoropiperidin-4-yl)ethan-1-ol

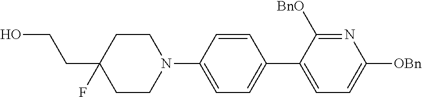 [SMILES: OCCC1(F)CCN(c2ccc(-c3ccc(OCc4ccccc4)nc3OCc3ccccc3)cc2)CC1]

Three batches in parallel.

To a solution of 2,6-bis(benzyloxy)-3-(4-bromophenyl)pyridine (14.5 g, 32.6 mmol, 1.20 eq) and 2-(4-fluoropiperidin-4-yl)ethan-1-ol hydrochloride (5.00 g, 27.2 mmol, 1.00 eq, HCl) in dioxane (50.0 mL) was added Cs₂CO₃ (26.6 g, 81.6 mmol, 3.00 eq) and XPhos Pd G3 (1.15 g, 1.36 mmol, 0.05 eq), and the reaction mixture was stirred for 20 hrs at 90° C. under nitrogen atmosphere. The reaction mixture was cooled to 30° C. and diluted with water (450 mL), extracted with ethyl acetate (120 mL*2). The combined organic phase was washed with brine (100 mL*2), dried over Na₂SO₄, concentrated in vacuo. The residue was purified by column chromatography (SiO₂, Petroleum ether/Ethyl acetate=10/1 to 1/2, Petroleum ether/Ethyl acetate=1/1, R_{f}=0.25). 2-(1-(4-(2,6-bis(benzyloxy)pyridin-3-yl)phenyl)-4-fluoropiperidin-4-yl)ethan-1-ol (12.0 g, 22.8 mmol, 27.9% yield, 97.4% purity) was obtained as brown oil. LCMS: Rt=0.586 min, m/z=513.3 (M+H)⁺. ¹H NMR (400 MHz, DMSO-d₆): δ 7.68-7.66 (m, 1H), 7.43-7.30 (m, 12H), 6.97-6.95 (m, 2H), 6.51-6.49 (m, 1H), 5.39-5.35 (d, J=18.4 Hz, 4H), 4.49 (t, J=4.8 Hz, 1H), 3.60-3.50 (m, 4H), 2.99-2.92 (m, 2H), 1.86-1.70 (m, 6H)

### Step 4: rac-(R)-3-(4-(4-fluoro-4-(2-hydroxyethyl)piperidin-1-yl)phenyl)piperidine-2,6-dione

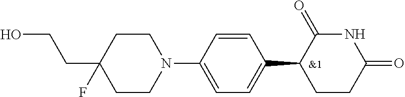 [SMILES: O=C1CC[C@@H](c2ccc(N3CCC(F)(CCO)CC3)cc2)C(=O)N1]

To a solution of 2-(1-(4-(2,6-bis(benzyloxy)pyridin-3-yl)phenyl)-4-fluoropiperidin-4-yl)ethan-1-ol (18.3 g, 35.7 mmol, 1.00 eq) in THF (183 mL) and dioxane (183 mL) was added Pd/C (9.15 g, 8.60 mmol, 10.0% purity, 2.41e-1.00 eq) and Pd(OH)₂ (9.15 g, 3.26 mmol, 5.00% purity, 0.09 eq) under N₂ atmosphere. The suspension was degassed and purged with H₂ for 3 times. The mixture was stirred under H₂ (15 Psi) at 15-25° C. for 4 hrs. The reaction mixture was filtered to collect the filtrate. The filtrate was concentrated in vacuo. rac-(R)-3-(4-(4-fluoro-4-(2-hydroxyethyl)piperidin-1-yl)phenyl)piperidine-2,6-dione (9.15 g, 25.9 mmol, 72.6% yield, 94.8% purity) was obtained as a gray solid. LCMS: Rt=0.354 min, m/z=335.2 (M+H)*. ¹H NMR (400 MHz, DMSO-d₆): δ 10.7 (s, 1H), 7.05-7.03 (m, 2H), 6.92-6.90 (m, 2H), 4.48 (t, J=4.8 Hz, 1H), 3.74-3.70 (m, 1H), 3.59-3.54 (m, 2H), 3.47-3.44 (m, 2H), 2.95-2.88 (m, 2H), 2.67-2.58 (m, 1H), 2.47-2.43 (m, 1H), 2.18-2.07 (m, 1H), 2.03-1.96 (m, 1H), 1.85-1.74 (m, 6H).

### Step 5: rac-(R)-2-(1-(4-(2,6-dioxopiperidin-3-yl)phenyl)-4-fluoropiperidin-4-yl)acetaldehyde

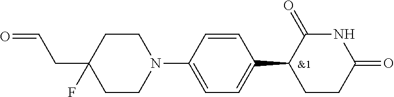 [SMILES: O=CCC1(F)CCN(c2ccc([C@@H]3CCC(=O)NC3=O)cc2)CC1]

To a mixture of rac-(R)-3-(4-(4-fluoro-4-(2-hydroxyethyl)piperidin-1-yl)phenyl)piperidine-2,6-dione (9.15 g, 27.3 mmol, 1.00 eq) in DMSO (91.0 mL) was added IBX (15.3 g, 54.7 mmol, 2.00 eq) at 15-25° C. and the resulting mixture was stirred at 15-25° C. for 2 hrs. The mixture was poured into H₂O (270 mL), extracted with DCM (100 mL*3), the combined organic layers were washed with Na₂S₂O₃ (80.0 mL), sat. NaHCO₃ (80.0 mL), brine (80.0 mL), dried with Na₂SO₄, filtered and concentrated in vacuum. The crude product was triturated with solvent 1 (Petroleum ether/Ethyl acetate=5/1, 12.0 mL) at 15-25° C. for 30 mins. rac-(R)-2-(1-(4-(2,6-dioxopiperidin-3-yl)phenyl)-4-fluoropiperidin-4-yl)acetaldehyde (9.03 g, 25.6 mmol, 93.4% yield, 94.1% purity) was obtained as a yellow solid. MS: m/z=333.1 (M+H)⁺. ¹H NMR (400 MHz, DMSO-d₆): δ 10.7 (s, 1H), 9.80-9.79 (m, 1H), 7.07-7.05 (m, 2H), 6.95-6.93 (m, 2H), 3.75-3.71 (m, 1H), 3.52-3.49 (m, 2H), 3.01-2.95 (m, 2H), 2.85-2.79 (m, 2H), 2.68-2.59 (, 1H), 2.48-2.44 (m, 1H), 2.19-2.08 (m, 1H), 2.04-1.96 (m, 4H), 1.93-1.83 (m, 1H).

### Intermediate 38

### RAC-(R)-2-(1-(4-(2,6-DIOXOPIPERIDIN-3-YL)-3-FLUOROPHENYL)-4-FLUOROPIPERIDIN-4-YL)ACETALDEHYDE

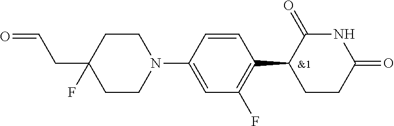 [SMILES: O=CCC1(F)CCN(c2ccc([C@@H]3CCC(=O)NC3=O)c(F)c2)CC1]

### Step 1: 2-(1-(4-bromo-3-fluorophenyl)-4-fluoropiperidin-4-yl)ethan-1-ol

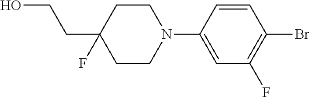 [SMILES: OCCC1(F)CCN(c2ccc(Br)c(F)c2)CC1]

To a solution of 1-bromo-2-fluoro-4-iodobenzene (25.0 g, 83.1 mmol, 1.00 eq) and 2-(4-fluoropiperidin-4-yl)ethan-1-ol hydrochloride (18.3 g, 99.7 mmol, 1.20 eq, HCl) in DMSO (250 mL) was added K₂CO₃ (23.0 g, 166 mmol, 2.00 eq), L-PROLINE (3.83 g, 33.2 mmol, 0.40 eq) and CuI (3.16 g, 16.6 mmol, 0.20 eq) under N₂ at 25° C. Then the mixture was heated to 80° C. and stirred at 80° C. for 12 hrs. The mixture was poured into H₂O (800 mL), extracted with EtOAc (300 mL*2), the combined organic layers were washed with brine (800 mL), dried with Na₂SO₄, filtered and concentrated in vacuum. The residue was purified by prep-HPLC (column: Phenomenex luna C18 (250*70 mm, 10 um); mobile phase: [H₂O (0.05% HCl)-ACN]; gradient: 40%-60% B over 25.0 min) and extracted with EtOAc (50.0 mL*2), the organic layer was washed with brine (100 mL), dried over anhydrous Na₂SO₄, filtered and concentrated. 2-(1-(4-bromo-3-fluorophenyl)-4-fluoropiperidin-4-yl)ethan-1-ol (3.60 g, 11.1 mmol, 34.5% yield, 98.9% purity) was obtained as yellow oil. LCMS: RT=0.528 min, m/z=320.1 (M+H)⁺. ¹H NMR (400 MHz, DMSO-d6): δ 7.42-7.38 (m, 1H), 6.96-6.92 (m, 2H), 6.73-6.37 (m, 1H), 4.48 (s, H), 3.62-3.49 (m, 4H), 3.02-2.95 (m, 2H), 1.84-1.69 (m, 6H).

### Step 2: 2-(1-(4-(2,6-bis(benzyloxy)pyridin-3-yl)-3-fluorophenyl)-4-fluoropiperidin-4-yl)ethan-1-ol

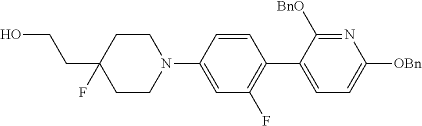 [SMILES: OCCC1(F)CCN(c2ccc(-c3ccc(OCc4ccccc4)nc3OCc3ccccc3)c(F)c2)CC1]

To a solution of 2-(1-(4-bromo-3-fluorophenyl)-4-fluoropiperidin-4-yl)ethan-1-ol (3.50 g, 10.9 mmol, 1.00 eq) and 2,6-dibenzyloxy-3-(4,4,5,5-tetramethyl-1,3,2-dioxaborolan-2-yl)pyridine (5.47 g, 13.1 mmol, 1.20 eq) in dioxane (35.0 mL) and H₂O (7.00 mL) was added Pd(PPh₃)₄ (1.26 g, 1.09 mmol, 0.1 eq) and Cs₂CO₃ (8.90 g, 27.3 mmol, 2.50 eq) under N₂. The mixture was stirred at 100° C. for 12 hrs under N₂. The reaction mixture was cooled to 25° C. and poured into H₂O (100 mL), extracted with EtOAc (150 mL*2). The organic layer was washed with brine (200 mL), dried over anhydrous Na₂SO₄, filtered and concentrated. The residue was purified by column chromatography (SiO₂, Petroleum ether/Ethyl acetate=1/0 to 50/1, Petroleum ether/Ethyl acetate=3/1, R_{f}—0.24), then the mixture was concentrated under vacuum. 2-(1-(4-(2,6-bis(benzyloxy)pyridin-3-yl)-3-fluorophenyl)-4-fluoropiperidin-4-yl)ethan-1-ol (5.00 g, 8.91 mmol, 81.5% yield, 94.6% purity) was obtained as a brown oil. LCMS: RT=0.647 min, m/z=531.3 (M+H)⁺. ¹H NMR (400 MHz, DMSO-d6): δ 7.57 (d, J=4.0 Hz 1H), 7.43-7.31 (m, 10H), 6.81 (t, J=6.8 Hz 1H), 6.50 (d, J=6.8 Hz 1H), 6.35 (d, J=6.8 Hz 4H), 4.45 (t, J=6.8 Hz 1H), 3.59-3.52 (m, 4H), 3.04-2.95 (m, 2H), 1.86-1.73 (m, 6H).

### Step 3: rac-(R)-3-(2-fluoro-4-(4-fluoro-4-(2-hydroxyethyl)piperidin-1-yl)phenyl)piperidine-2,6-dione

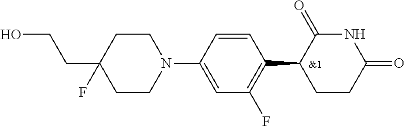 [SMILES: O=C1CC[C@@H](c2ccc(N3CCC(F)(CCO)CC3)cc2F)C(=O)N1]

To a solution of Pd/C (2.50 g, 2.35 mmol, 10.0% purity, 2.49e-1 eq) and Pd(OH)₂ (2.50 g, 8.90 mmol, 50.0% purity, 9.45e-1 eq) in dioxane (50.0 mL) was added 2-(1-(4-(2,6-bis(benzyloxy)pyridin-3-yl)-3-fluorophenyl)-4-fluoropiperidin-4-yl)ethan-1-ol (5.00 g, 9.42 mmol, 1.00 eq) in THF (50.0 mL) under N₂ at 25° C. The suspension was degassed and purged with H₂ for 3 times. The mixture was stirred under H₂ (50 Psi) at 25° C. for 12 hrs. The reaction mixture was filtered and the filtrate was concentrated. rac-(R)-3-(2-fluoro-4-(4-fluoro-4-(2-hydroxyethyl)piperidin-1-yl)phenyl)piperidine-2,6-dione (2.80 g, 7.68 mmol, 81.5% yield, 96.7% purity) was obtained as an off-white solid. LCMS: RT=0.409 min, m/z=353.2 (M+H)⁺. ¹H NMR (400 MHz, DMSO-d6): δ 10.80 (s, 1H), 7.07 (t, J=8.4 Hz 1H), 6.77-6.73 (m, 1H), 4.92 (t, J=5.2 Hz 1H), 3.72 (dd, J=4.8 11.8 Hz 4H), 3.57-3.51 (m, 4H), 3.00-2.82 (m, 2H), 2.70-2.67 (m, 1H), 2.57-2.53 (m, 1H), 2.25-2.18 (m, 1H), 2.07-1.96 (m, 1H), 1.85-1.69 (m, 6H).

### Step 4: rac-(R)-2-(1-(4-(2,6-dioxopiperidin-3-yl)-3-fluorophenyl)-4-fluoropiperidin-4-yl)acetaldehyde

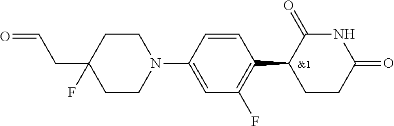 [SMILES: O=CCC1(F)CCN(c2ccc([C@@H]3CCC(=O)NC3=O)c(F)c2)CC1]

To a solution of rac-(R)-3-(2-fluoro-4-(4-fluoro-4-(2-hydroxyethyl)piperidin-1-yl)phenyl)piperidine-2,6-dione (2.80 g, 7.95 mmol, 1.00 eq) in DMSO (28.0 mL) was added IBX (3.34 g, 11.9 mmol, 1.50 eq) at 25° C. and the reaction mixture was stirred at 25° C. for 2 hrs. The mixture was quenched with 10% Na₂SO₃ solution (100 mL). The aqueous phase was extracted with EtOAc (60.0 mL*2). The combined organic phase was washed with brine (100 mL), dried with anhydrous Na₂SO₄, filtered and concentrated in vacuum. The residue was purified by prep-HPLC (column: Phenomenex luna C18 (250*70 mm, 10 um); mobile phase: [H₂O (0.05% HCl)-ACN]; gradient: 5%-40% B over 32.0 min) and extracted with EtOAc (100 mL*2), washed with brine (100 mL). The combined organic layers were dried over Na₂SO₄ and concentrated. rac-(R)-2-(1-(4-(2,6-dioxopiperidin-3-yl)-3-fluorophenyl)-4-fluoropiperidin-4-yl)acetaldehyde (1.23 g, 3.47 mmol, 43.7% yield, 98.9% purity) was obtained as a yellow solid. LCMS: RT=0.127 min, m/z=351.2 (M+H)⁺. ¹H NMR (400 MHz, DMSO-d6): δ 10.81 (s, 1H), 9.82 (s, 1H), 7.15-7.08 (m, 1H), 6.81-6.75 (m, 2H), 3.89 (dd, J=4.8 11.8 Hz 4H), 3.57 (d, J=12.8 Hz 1H), 3.06-2.99 (m, 2H), 2.82 (dd, J=1.60 12.8 Hz 2H), 2.71-2.68 (m, 1H), 2.56-2.53 (m, 1H), 2.28-2.12 (m, 1H), 2.07-1.96 (m, 4H), 1.85-1.77 (m, 1H).

### Intermediate 40

### RAC-(R)-2-(1-(1-(2,6-DIOXOPIPERIDIN-3-YL)-3-METHYL-2-OXO-2,3-DIHYDRO-1H-BENZO[D]IMIDAZOL-4-YL)PIPERIDIN-4-YL)ACETALDEHYDE

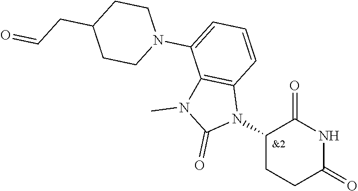 [SMILES: Cn1c(=O)n([C@H]2CCC(=O)NC2=O)c2cccc(N3CCC(CC=O)CC3)c21]

### Step 1: Synthesis of 3-(4-bromo-3-methyl-2-oxo-2,3-dihydro-1H-benzo[d]imidazol-1-yl)piperidine-2,6-dione

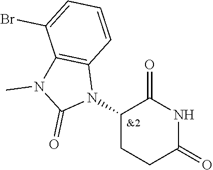 [SMILES: Cn1c(=O)n([C@H]2CCC(=O)NC2=O)c2cccc(Br)c21]

To a stirred solution of 7-bromo-1-methyl-1,3-dihydro-2H-benzo[d]imidazol-2-one (25 g, 110 mmol) in THF (100 mL) was added LiHMDS (1.5 M, 220 mL, 330 mmol) reaction mixture was stirred for 30 min. In another flask 3-bromopiperidine-2,6-dione (42.3 g, 220 mmol) in THF (80 mL) was taken and above solution was added slowly over a period of 20 minutes. Reaction mixture then stirred at 60° C. for 4 h. After completion of the reaction (monitored by TLC and LCMS), reaction mixture cooled down and quenched with aq. NH₄Cl solution (500 mL) and extracted with ethyl acetate (3×1000 mL). Combined organic layer was dried over anhydrous sodium sulphate, filtered and concentrated under reduced pressure to get crude product. The crude compound was diluted with IPA:heptane (1:1, 200 mL) and was heated to 50° C. for 30 min. Solid obtained was filtered and dried to get title compound as a grey solid (15 g, 40%). LCMS: m/z 337.90 [M+H]Purity: 87.36%.

### Step-2: Synthesis of 3-(4-(4-(2-((tert-butyldimethylsilyl)oxy)ethyl)piperidin-1-yl)-3-methyl-2-oxo-2,3-dihydro-1H-benzo[d]imidazol-1-yl)piperidine-2,6-dione

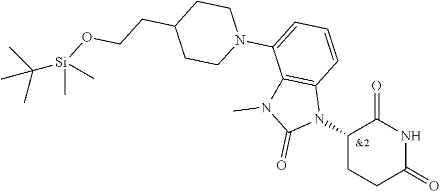 [SMILES: Cn1c(=O)n([C@H]2CCC(=O)NC2=O)c2cccc(N3CCC(CCO[Si](C)(C)C(C)(C)C)CC3)c21]

To a stirred solution of 3-(4-bromo-3-methyl-2-oxo-2,3-dihydro-1H-benzo[d]imidazol-1-yl)piperidine-2,6-dione (5 g, 14.8 mmol) and 4-(2-((tert-butyldimethylsilyl)oxy)ethyl)piperidine (3.6 g, 14.8 mmol) in Toluene (50 mL) was added LiHMDS solution (50 mL, 73.9 mmol) at RT, Nitrogen gas was purged into reaction mixture for 10 min. Ruphos (690 mg, 1.48 mmol) and RuPhos-Pd-G2 (1.15 g, 1.48 mmol) were added to reaction mixture and stirred at 100° C. for 3 h. After completion of the reaction (monitored by TLC and LCMS), Reaction mixture was concentrated under reduced pressure and crude obtained was purified by Flash column chromatography (60-80% EtOAc:heptane) to get title compound as brown solid (2.1 g, 20%). LCMS: m/z 501.20 [M+H]⁺; Purity: 68.39%

### Step-3: Synthesis of 3-(4-(4-(2-hydroxyethyl)piperidin-1-yl)-3-methyl-2-oxo-2,3-dihydro-1H-benzo[d]imidazol-1-yl)piperidine-2,6-dione

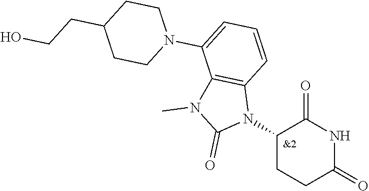 [SMILES: Cn1c(=O)n([C@H]2CCC(=O)NC2=O)c2cccc(N3CCC(CCO)CC3)c21]

To an ice cold stirred solution of 3-(4-(4-(2-((tert-butyldimethylsilyl)oxy)ethyl)piperidin-1-yl)-3-methyl-2-oxo-2,3-dihydro-1H-benzo[d]imidazol-1-yl)piperidine-2,6-dione (2.1 g, 4.19 mmol) in CH₃CN (16 mL) and water (4 mL) was added TFA (1.5 mL) and stirred at RT for 3 h. After completion of reaction (monitored by TLC and LCMS), reaction mixture was concentrated and quenched with water (20 mL) and extracted with DCM (2×25 mL). Combined organic layer was dried over anhydrous sodium sulphate, filtered and concentrated under reduced pressure to get crude product. The crude product was purified by flash column chromatography (3-5% MeOH in DCM) to get the title compound as white solid (700 mg, 44%). LCMS: m/z 387.05[M+H]⁺; Purity: 99.87%.

### Step-4: Synthesis of the Title Compound

To an ice-cold stirred solution of 3-(4-(4-(2-hydroxyethyl)piperidin-1-yl)-3-methyl-2-oxo-2,3-dihydro-1H-benzo[d]imidazol-1-yl)piperidine-2,6-dione (0.7 g, 1.55 mmol) in DCM (10 mL) were added DMP (1.15 g, 2.72 mmol) and NaHCO₃ (0.761 g, 9.06 mmol). The reaction mixture was stirred at 15° C. for 1 h. After completion of reaction (monitored by TLC and LCMS), the reaction mixture was quenched with water (20 mL) and extracted with 10% MeOH:DCM (2×100 mL). The combined organic layer was dried over anhydrous sodium sulphate, filtered and concentrated under reduced pressure to get the crude product. The crude product was purified by flash column chromatography (5-8% MeOH in DCM) to get the title compound as yellow solid (447 mg, 64%). LCMS: m/z 385.15 [M+H]⁺; Purity: 99.00% ¹H NMR (400 MHz, DMSO-d₆): δ 11.08 (s, 1H), 9.72-9.71 (m, 1H), 6.99-6.95 (m, 1H), 6.91-6.85 (m, 2H), 5.36-5.32 (m, 1H), 3.61 (s, 3H), 3.09-3.07 (m, 2H), 2.88-2.84 (m, 1H), 2.73-2.63 (m, 4H), 2.49-2.44 (m, 2H), 1.99-1.96 (m, 2H), 1.77-1.74 (m, 2H), 1.47-1.39 (m, 2H).

### Intermediate 41

### 3-FLUORO-1-(6-(2-(METHOXYMETHOXY)PHENYL)PYRIDAZIN-4-YL)PIPERIDINE-3-CARBOXYLIC ACID LITHIUM SALT

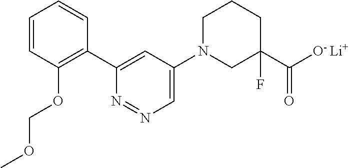 [SMILES: COCOc1ccccc1-c1cc(N2CCCC(F)(C(=O)[O-])C2)cnn1 ; [Li+]]

### Step-1: 1-(tert-butyl) 3-methyl 3-fluoropiperidine-1,3-dicarboxylate

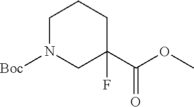 [SMILES: COC(=O)C1(F)CCCN(C(=O)OC(C)(C)C)C1]

To a stirred solution 1-(tert-butyl) 3-methyl piperidine-1,3-dicarboxylate (10 g, 41.1 mmol) in THF (0.1 L) was added LiHMDS solution (1M in THF, 61.7 mL, 61.7 mmol) at −78°. The reaction mixture was stirred at −78° C. to RT for 2 h. Then, NFSI (25.9 g, 82.2 mmol) was added at −78°. The mixture was stirred at RT for 4 h. The reaction mixture was quenched by the addition of saturated NH₄Cl solution (0.5 L) and extracted with EtOAc (2×0.5 L). The combined organic layer was washed with water (0.5 L), brine solution (0.5 L), dried over anhydrous Na₂SO₄, filtered concentrated under reduced pressure to get the crude compound. The crude compound was purified by flash column chromatography (10-20% EtOAc in heptane) to get 1-(tert-butyl) 3-methyl 3-fluoropiperidine-1,3-dicarboxylate as yellow sticky liquid (4.5 g, 42%). ¹H NMR (400 MHz, CDCl₃): δ 4.03-3.92 (m, 1H), 3.87-3.84 (m, 1H), 3.74 (s, 3H), 3.70-3.50 (m, 2H), 3.03-2.75 (m, 1H), 1.99-1.85 (m, 2H), 1.60-1.50 (m, 1H), and 1.38 (s, 9H).

### Step-2: methyl 3-fluoropiperidine-3-carboxylate

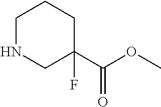

To an ice-cold solution of 1-(tert-butyl) 3-methyl 3-fluoropiperidine-1,3-dicarboxylate 2 (4.5 g, 17.2 mmol) in DCM (45 mL) was added TFA (15 mL). The reaction mixture was stirred at RT for 8 h. After completion of reaction (monitored by TLC and LCMS), the reaction mixture was concentrated under reduced pressure. The crude compound was triturated with Diethyl ether (50 mL) and Pentane (50 mL) and dried to get methyl 3-fluoropiperidine-3-carboxylate as an off white solid (3 g, Crude).

LCMS: m/z 162.30 [M+H]⁺; Purity: 80%.

### Step-3: methyl 1-(6-chloropyridazin-4-yl)-3-fluoropiperidine-3-carboxylate

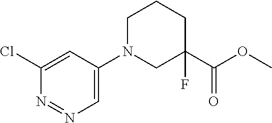 [SMILES: COC(=O)C1(F)CCCN(c2cnnc(Cl)c2)C1]

To a stirred solution of methyl 3-fluoropiperidine-3-carboxylate (3 g, 18.6 mmol) in CH₃CN (30 mL) was added 3,5-dichloropyridazine (3.05 g, 20.5 mmol) and DIPEA (7.22 mL, 55.8 mmol) at RT under inert atmosphere. The reaction mixture was stirred at 80° C. for 16 h. After completion of reaction (monitored by TLC and LCMS), the reaction mixture was concentrated under reduced pressure to get the crude compound. The crude compound was purified by flash column chromatography (5-10% MeOH in DCM) to get methyl 1-(6-chloropyridazin-4-yl)-3-fluoropiperidine-3-carboxylate as yellow sticky solid (2.7 g, 57% over two steps).

LCMS: m/z 274.10 [M+H]⁺; Purity: 99.47%.

### Step-4: methyl 3-fluoro-1-(6-(2-(methoxymethoxy)phenyl)pyridazin-4-yl)piperidine-3-carboxylate

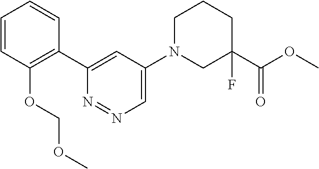 [SMILES: COCOc1ccccc1-c1cc(N2CCCC(F)(C(=O)OC)C2)cnn1]

To a stirred solution methyl 1-(6-chloropyridazin-4-yl)-3-fluoropiperidine-3-carboxylate (2.7 g, 9.86 mmol) and (2-(methoxymethoxy)phenyl)boronic acid (2.15 g, 11.8 mmol) in 1,4-Dioxane (40 mL) was added K₃PO₄ (3.14 g, 14.8 mmol). and the reaction mixture was purged with nitrogen for 15 minutes. XPhosPdG₃ (651 mg, 0.77 mmol) was then added to the reaction mixture and stirred at 70° C. for 4 h. After completion, the reaction mixture was filtered through celite pad. Filtrate was quenched with water (100 mL) and extracted with EtOAc (2×100 mL). The combined organic layer was washed with brine (100 mL), dried over anhydrous sodium sulphate, filtered and concentrated under reduced pressure. The crude was purified by flash column chromatography (80 to 90% EtOAc in heptane) to get methyl 3-fluoro-1-(6-(2-(methoxymethoxy)phenyl)pyridazin-4-yl)piperidine-3-carboxylate as brown sticky solid (2 g, 54%). LCMS: m/z 376.50 [M+H]⁺; Purity: 96.22%.

### Step-5: 3-fluoro-1-(6-(2-(methoxymethoxy)phenyl)pyridazin-4-yl)piperidine-3-carboxylic acid (lithium salt)

To an ice-cold solution of methyl 3-fluoro-1-(6-(2-(methoxymethoxy)phenyl)pyridazin-4-yl)piperidine-3-carboxylate (2 g, 5.33 mmol) in THF (10 mL) and Water (5 mL) was added LiOH·H2O (0.23 g, 5.33 mmol). The reaction mixture stirred at RT for 4 h. The reaction was concentrated under reduced pressure, diluted with 25% IPA in CHCl3 (100 mL) and filtered through a sintered glass funnel. Filtrate was concentrated under reduced pressure. The crude was triturated with Diethyl ether (30 mL) and Pentane (30 mL) to get the title compound as an off white solid (1.51 g, 77%). LCMS: m/z 362.30 [M+H]⁺; Purity: 97.03%. ¹H NMR (400 MHz, DMSO-d₆): δ 8.85 (d, J=2.8 Hz, 1H), 7.63 (dd, J=2.0 Hz, 7.6 Hz, 1H), 7.42-7.38 (m, 1H), 7.22 (d, J=7.9 Hz, 1H), 7.14-7.09 (m, 2H), 5.20 (s, 2H), 4.04-3.98 (m, 2H), 3.50-3.38 (m, 1H), 3.33 (s, 3H), 3.04-2.98 (m, 1H), 2.11-1.94 (m, 1H), 1.86-1.80 (m, 1H), 1.72-1.62 (m, 1H) and 1.58-1.54 (m, 1H).

### Intermediate 42

### 3-CYCLOBUTOXY-1-(6-(2-(METHOXYMETHOXY)PHENYL)PYRIDAZIN-4-YL)PYRROLIDINE-3-CARBOXYLIC ACID LITHIUM SALT

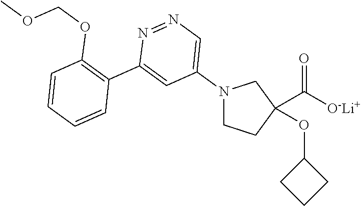

### Step-1: methyl 2-cyclobutoxyacrylate

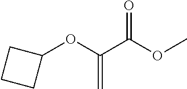

To a stirred solution of methyl 2-methoxyacrylate (5 g, 43.1 mmol) in Benzene (100 mL) were added cyclobutanol (6.45 ml, 64.6 mmol), activated powdered 4 Å MS (4 g) and NaH₂PO₄ (4.13 g, 34.4 mmol). The reaction mixture was degassed thrice with N₂ gas. Then CuCl₂ (347 mg, 2.58 mmol) and Bis(benzonitrile)palladium (II) Dichloride (1.65 g, 4.31 mmol) were added, and the reaction mixture was stirred at RT for 16 h. After completion, charcoal (2 g) was added to the reaction mixture and stirred at RT for another 30 min. Then the reaction mixture was filtered through sintered funnel and washed with Diethyl ether (100 mL). Filtrate was diluted with water (50 mL) and organic layer was separated. The combined organic layer was dried over anhydrous Na₂SO₄, filtered and concentrated under reduced pressure (below 30° C.) to get methyl 2-cyclobutoxyacrylate as colorless liquid (3.7 g, 55%). 1H NMR (400 MHz, CDCl₃): δ 5.33 (d, J=2.0 Hz, 1H), 4.50 (d, J=2.0 Hz, 1H), 4.47-4.40 (m, 1H), 3.82 (s, 3H), 2.23-2.13 (m, 4H) and 1.90-1.82 (m, 2H).

### Step-2: methyl 1-benzyl-3-cyclobutoxypyrrolidine-3-carboxylate

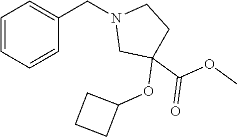 [SMILES: COC(=O)C1(OC2CCC2)CCN(Cc2ccccc2)C1]

To a stirred solution of methyl 2-cyclobutoxyacrylate (3.7 g, 23.7 mmol) and N-benzyl-1-methoxy-N-((trimethylsilyl)methyl)methanamine (11.2 g, 47.4 mmol) in Acetonitrile (50 mL) was added LiF (1.84 g, 71.1 mmol) at RT and the reaction mixture was stirred at 70° C. for 16 h. After completion, the reaction mixture was concentrated under reduced pressure to get a crude residue which was purified by flash column chromatography (5% EtOAc in heptane) to get methyl 1-benzyl-3-cyclobutoxypyrrolidine-3-carboxylate as pale yellow sticky gum (3 g, 44%). LCMS: m/z 290.00 [M+H]⁺; Purity: 76%.

### Step-3: methyl 3-cyclobutoxypyrrolidine-3-carboxylate

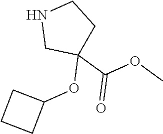

To a stirred solution of methyl 1-benzyl-3-cyclobutoxypyrrolidine-3-carboxylate (3 g, 10.4 mmol) in MeOH (30 mL) was added 10% Pd/C (1.5 g, w/w) followed by HCOONH₄ (3.27 g, 51.8 mmol) at RT and the reaction mixture was stirred at 65° C. for 5 h. After completion, the reaction mixture was filtered through celite pad and washed with 30% MeOH in DCM. The filtrate was concentrated under reduced pressure to get methyl 3-cyclobutoxypyrrolidine-3-carboxylate as colorless liquid (1.4 g, 68%). LCMS: m/z 200.18 M+H]⁺; Purity: 70%.

### Step-4: methyl 1-(6-chloropyridazin-4-yl)-3-cyclobutoxypyrrolidine-3-carboxylate

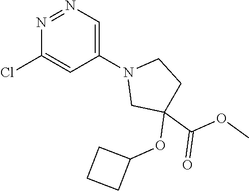 [SMILES: COC(=O)C1(OC2CCC2)CCN(c2cnnc(Cl)c2)C1]

To a stirred solution of methyl 3-cyclobutoxypyrrolidine-3-carboxylate (1.9 g, 6.48 mmol) and 3,5-dichloropyridazine (966 mg, 6.48 mmol) in Acetonitrile (20 mL) was added DIPEA (5.65 mL, 32.4 mmol) and the reaction mixture was stirred at 80° C. for 16 h. After completion of the reaction (monitored by LCMS and TLC), reaction mixture was concentrated under reduced pressure. The crude was purified by flash column chromatography (3% MeOH in DCM) to get methyl 1-(6-chloropyridazin-4-yl)-3-cyclobutoxypyrrolidine-3-carboxylate as white solid (1.1 g, 54%). LCMS: m/z 312.77 [M+H]⁺; Purity: 99%

### Step-5: methyl 3-cyclobutoxy-1-(6-(2-(methoxymethoxy)phenyl)pyridazin-4-yl)pyrrolidine-3-carboxylate

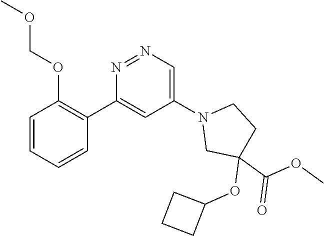

To a stirred solution of methyl 1-(6-chloropyridazin-4-yl)-3-cyclobutoxypyrrolidine-3-carboxylate (1 g, 3.21 mmol) and (2-(methoxymethoxy)phenyl)boronic acid (700 mg, 3.85 mmol) in 1,4-Dioxane:water (4:1, 10 mL) was added K₃PO₄ (1.02 g, 4.81 mmol) and the reaction mixture was purged with nitrogen gas for 15 minutes. Afterwards, XPhosPdG₃ (272 mg, 0.32 mmol) was then added to the reaction mixture and stirred at 70° C. for 4 h. After completion, the reaction mixture was filtered through celite bed, filtrate was diluted with water (50 mL) and extracted with EtOAc (2×50 mL). The combined organic layer was washed with brine (50 mL), dried over anhydrous sodium sulphate, filtered and concentrated under reduced pressure to get crude. The crude was purified by flash column chromatography (80 to 90% EtOAc in Heptane) to get methyl 3-cyclobutoxy-1-(6-(2-(methoxymethoxy)phenyl)pyridazin-4-yl)pyrrolidine-3-carboxylate as brown liquid (900 mg, 68%). LCMS: m/z 414.11 [M+H]⁺; Purity: 99%.

### Step-6: 3-cyclobutoxy-1-(6-(2-(methoxymethoxy)phenyl)pyridazin-4-yl)pyrrolidine-3-carboxylic acid (Lithium salt)

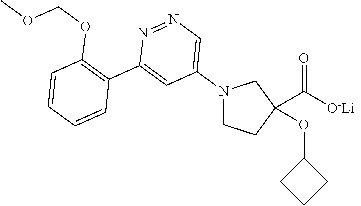

To a stirred solution of methyl 3-cyclobutoxy-1-(6-(2-(methoxymethoxy)phenyl)pyridazin-4-yl)pyrrolidine-3-carboxylate (900 mg, 2.18 mmol) in THF (6 mL) was added solution of LiOH·H₂O (110 mg, 2.61 mmol) in water (3 mL) and the resultant reaction mass was stirred at RT for 5 h. After completion of reaction (monitored by TLC and LCMS), reaction mixture was concentrated under reduced pressure, diluted with 25% IPA in CHCl₃ (50 mL) and filtered through sintered funnel. Filtrate was concentrated under reduced pressure to get 3-cyclobutoxy-1-(6-(2-(methoxymethoxy)phenyl)pyridazin-4-yl)pyrrolidine-3-carboxylic acid (Lithium salt) as an off-white solid (810 mg, 93%). LCMS: m/z 400.37 [M+H]+; Purity: 99.73%. ¹H NMR (400 MHz, DMSO-d₆): δ 8.51 (s, 1H), 7.64 (dd, J=1.6 Hz, 7.6 Hz, 1H), 7.41-7.37 (m, 1H), 7.22 (d, J=8.0 Hz, 1H), 7.12-7.09 (m, 1H), 6.80 (s, 1H), 5.22 (s, 2H), 4.24-4.16 (m, 1H), 3.61 (brs, 1H), 3.40 (brs, 1H), 3.33 (s, 3H), 3.31-3.22 (m, 2H), 2.33-2.25 (m, 1H), 2.13-2.07 (m, 2H), 1.98-1.94 (m, 1H), 1.86-1.78 (m, 1H), 1.76-1.64 (m, 1H), 1.50-1.43 (m, 1H) and 1.37-1.27 (m, 1H).

### EXAMPLES

### Example 1

### N-(1-(2-(1-(4-(2,6-DIOXOPIPERIDIN-3-YL)-3-FLUOROPHENYL)PIPERIDIN-4-YL)ETHYL)PIPERIDIN-4-YL)-1-(6-(2-HYDROXYPHENYL)PYRIDAZIN-4-YL)-N-ISOBUTYL-4-METHOXYPIPERIDINE-4-CARBOXAMIDE

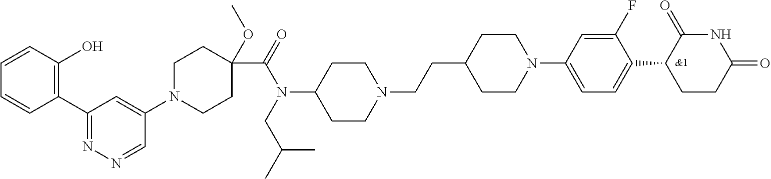 [SMILES: COC1(C(=O)N(CC(C)C)C2CCN(CCC3CCN(c4ccc([C@H]5CCC(=O)NC5=O)c(F)c4)CC3)CC2)CCN(c2cnnc(-c3ccccc3O)c2)CC1]

### Step 1: Synthesis of 1-(6-(2-hydroxyphenyl)pyridazin-4-yl)-N-isobutyl-4-methoxy-N-(piperidin-4-yl)piperidine-4-carboxamide

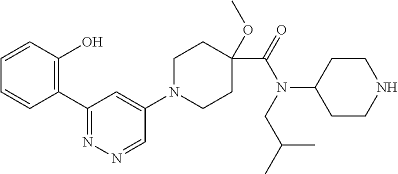 [SMILES: COC1(C(=O)N(CC(C)C)C2CCNCC2)CCN(c2cnnc(-c3ccccc3O)c2)CC1]

To a solution of lithio 4-methoxy-1-{6-[2-(methoxymethoxy)phenyl]pyridazin-4-yl}piperidine-4-carboxylate (50.0 mg, 0.13 mmol) and tert-butyl 4-[(2-methylpropyl)amino]piperidine-1-carboxylate (50.69 μL, 50.69 mg, 0.20 mmol) in DCM (1.00 mL) was added N,N-diisopropylethylamine (90.09 μL, 0.53 mmol). Then, bis(2-oxo-1,3-oxazolidin-3-yl)phosphinoyl chloride (100.66 mg, 0.39 mmol) was added and the reaction mixture was stirred at 40° C. overnight. Then, the reaction was quenched with water, extracted with DCM, dried, filtered and concentrated under reduced pressure. The crude material was dissolved in 2.00 mL of THF and 4M HCl in dioxane (1.00 mL) was added in one portion. The reaction was stirred at RT until complete by LCMS and then concentrated in vacuo to yield the desired product as a solid. LCMS: C₂₆H₃₇N₅O₃ requires: 467.3, found: m/z=468.4 [M+H]⁺.

### Step 2: Synthesis of the Title Compound

1-(6-(2-hydroxyphenyl)pyridazin-4-yl)-N-isobutyl-4-methoxy-N-(piperidin-4-yl)piperidine-4-carboxamide hydrochloride (30.0 mg, 0.06 mmol) was dissolved in DMA (1.00 mL) and rac-2-(1-{4-[(3R)-2,6-dioxopiperidin-3-yl]-3-fluorophenyl}piperidin-4-yl)acetaldehyde (23.74 mg, 0.07 mmol) was added in one portion. N,N-diisopropylethylamine (51.97 μL, 0.04 g, 0.29 mmol) was then added and the mixture was stirred for 30 min. Then, sodium triacetoxyborohydride (25.23 mg, 0.12 mmol) was added. The resulting reaction was stirred at RT until complete by LCMS. Once complete, the solution was injected onto RP—FC and purified with a gradient of 5-95% MeCN in H₂O to yield the title compound (23.40 mg, 41%) as a white solid. LCMS: C₄₄H₅₈FN₇O₅ requires 783.4, found: m/z=784.4 [M+H]⁺.

### Example 2

### (R)—N-(1-(2-(1-(4-(2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-4-METHOXY-N-(2,2,2-TRIFLUOROETHYL)PIPERIDINE-4-CARBOXAMIDE

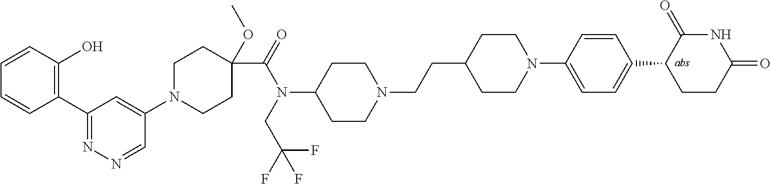 [SMILES: COC1(C(=O)N(CC(F)(F)F)C2CCN(CCC3CCN(c4ccc([C@H]5CCC(=O)NC5=O)cc4)CC3)CC2)CCN(c2cnnc(-c3ccccc3O)c2)CC1]

### Step 1: Synthesis of 1-(6-(2-hydroxyphenyl)pyridazin-4-yl)-4-methoxy-N-(piperidin-4-yl)-N-(2,2,2-trifluoroethyl)piperidine-4-carboxamide

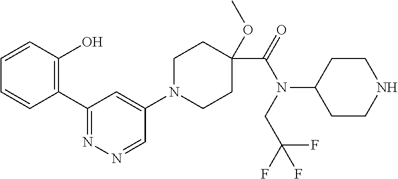 [SMILES: COC1(C(=O)N(CC(F)(F)F)C2CCNCC2)CCN(c2cnnc(-c3ccccc3O)c2)CC1]

Lithio 4-methoxy-1-{6-[2-(methoxymethoxy)phenyl]pyridazin-4-yl}piperidine-4-carboxylate (100.00 mg, 0.26 mmol) was dissolved in dry MeCN (4.00 mL). The flask was cooled to 0° C. and a drop of DMF was added. Then, oxalyl chloride (90.4 μL, 133.8 mg, 1.05 mmol) was added dropwise as a 100 μL/mL solution in MeCN. The reaction mixture was stirred at RT until complete by LCMS. Then, the reaction mixture was concentrated to approximately half of the initial volume. The acid chloride concentrated solution was then filtered through a syringe filter directly into an ice cold solution of tert-butyl 4-[(2,2,2-trifluoroethyl)amino]piperidine-1-carboxylate (111.63 mg, 0.39 mmol) and N,N-diisopropylethylamine (138.1 μL, 102.1 mg, 0.79 mmol) in MeCN (0.25 mL). The reaction was slowly warmed to RT and then warmed to 50° C. and stirred until complete by LCMS (4 h). Then, the reaction was quenched with water, washed with a saturated solution of K₂CO₃, extracted with DCM, dried, filtered and concentrated under reduced pressure. The crude material was dissolved in 1.00 mL of DCM and 4M HCl in dioxane (1.00 mL) was added in one portion. The reaction was stirred at RT until complete by LCMS and then concentrated in vacuo to yield the desired product as a yellow oil. LCMS: C₂₄H₃₀F₃N₅O₃ requires: 493.2, found: m/z=494.4 [M+H]⁺.

### Step 2: Synthesis of the Title Compound

1-(6-(2-hydroxyphenyl)pyridazin-4-yl)-4-methoxy-N-(piperidin-4-yl)-N-(2,2,2-trifluoroethyl)piperidine-4-carboxamide hydrochloride (14.00 mg, 0.03 mmol) was dissolved in DMA (0.50 mL) and 2-(1-{4-[(3R)-2,6-dioxopiperidin-3-yl]phenyl}piperidin-4-yl)acetaldehyde (8.30 mg, 0.03 mmol) was added in one portion. N,N-diisopropylethylamine (23.07 μL, 0.02 g, 0.13 mmol) was then added and the mixture was stirred for 30 min. Then, sodium triacetoxyborohydride (11.2 mg, 0.05 mmol) was added. The resulting reaction was stirred at RT until complete by LCMS. Once complete, the solution was injected onto RP—FC and purified with a gradient of 5-95% MeCN in H₂O to yield the title compound (5.10 mg, 23% yield) as a white solid LCMS: C₄₂H₅₂F₃N₇O₅ requires 791.4, found: m/z=792.4 [M+H]⁺.

### Example 3

### N-(1-((R)-2-(1-(4-((R)-2,6-DIOXOPIPERIDIN-3-YL)PHENYL)PIPERIDIN-4-YL)PROPYL)PIPERIDIN-4-YL)-N-ETHYL-1-(6-(2-HYDROXYPHENYL)PYRIDAZIN-4-YL)-4-METHOXYPIPERIDINE-4-CARBOXAMIDE

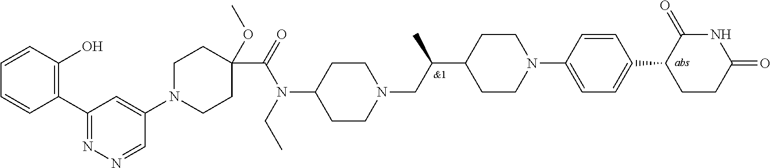 [SMILES: CCN(C(=O)C1(OC)CCN(c2cnnc(-c3ccccc3O)c2)CC1)C1CCN(C[C@@H](C)C2CCN(c3ccc([C@H]4CCC(=O)NC4=O)cc3)CC2)CC1]

### Step 1: Synthesis of N-ethyl-1-(6-(2-hydroxyphenyl)pyridazin-4-yl)-4-methoxy-N-(piperidin-4-yl)piperidine-4-carboxamide

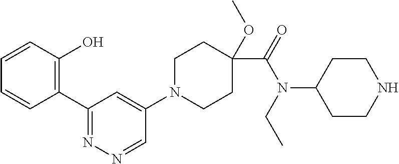 [SMILES: CCN(C(=O)C1(OC)CCN(c2cnnc(-c3ccccc3O)c2)CC1)C1CCNCC1]

To a solution of lithio 4-methoxy-1-{6-[2-(methoxymethoxy)phenyl]pyridazin-4-yl}piperidine-4-carboxylate (1.00 g, 2.63 mmol) and tert-butyl 4-(ethylamino)piperidine-1-carboxylate (0.90 g, 3.95 mmol) in DCM (15.00 mL) was added N,N-diisopropylethylamine (1.84 mL, 1.36 g, 10.5 mmol). Then bis(2-oxo-1,3-oxazolidin-3-yl)phosphinoyl chloride (2.01 g, 7.90 mmol) was added and the reaction mixture was stirred at 40° C. overnight. Then, the reaction was quenched with water, extracted with DCM 3×50 mL, dried, filtered and concentrated under reduced pressure. The crude material was purified via chromatography (MeOH:DCM, gradient) to yield the desired product as an orange foam. The material was dissolved in 10 mL methanol and 4M HCl in dioxane (10.0 mL) was added in one portion. The reaction was stirred at RT for 1 hour and then concentrated in vacuo to yield the desired product (1.25 g, 99%) as a white solid. LCMS: C₂₄H₃₃N₅O₃ requires: 439.2, found: m/z=440.2 [M+H]⁺

### Step 2: Synthesis of the Title Compound

N-ethyl-1-[6-(2-hydroxyphenyl)pyridazin-4-yl]-4-methoxy-N-(piperidin-4-yl)piperidine-4-carboxamide hydrochloride (30.0 mg, 0.063 mmol) was dissolved in DMA (1.00 mL) and 2-(1-{4-[(3R)-2,6-dioxopiperidin-3-yl]phenyl}piperidin-4-yl)propanal (20.7 mg, 0.063 mmol) was added in one portion. N,N-diisopropylethylamine (0.06 mL, 40.73 mg, 0.3151 mmol) was then added followed by sodium triacetoxyborohydride (40.1 mg, 0.189 mmol). The resulting reaction was stirred at RT for 1-2 hrs and monitored via LCMS. Once complete, the reaction was concentrated in vacuo. The residue was dissolved in DMF and purified via RP-HPLC to yield the title compound (10.2 mg, 18.8% yield) as a tan solid. LCMS: C₄₃H₅₇N₇O₅ requires: 751.4, found: m/z=752.3 [M+H]⁺

### Example 4

### N-(1-((RS)-1-(1-(4-((R)-2,6-DIOXOPIPERIDIN-3-YL)PHENYL)PIPERIDIN-4-YL)PROPAN-2-YL)PIPERIDIN-4-YL)-1-(6-(2-HYDROXYPHENYL)PYRIDAZIN-4-YL)-4-METHOXY-N-ETHYLPIPERIDINE-4-CARBOXAMIDE

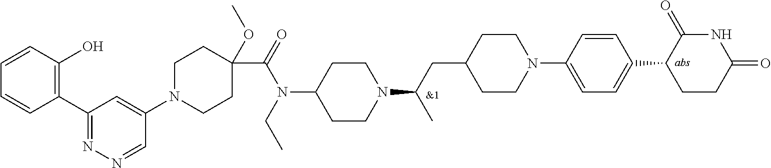 [SMILES: CCN(C(=O)C1(OC)CCN(c2cnnc(-c3ccccc3O)c2)CC1)C1CCN([C@H](C)CC2CCN(c3ccc([C@H]4CCC(=O)NC4=O)cc3)CC2)CC1]

### Step 1: Synthesis of the Title Compound

N-ethyl-1-[6-(2-hydroxyphenyl)pyridazin-4-yl]-4-methoxy-N-(piperidin-4-yl)piperidine-4-carboxamide hydrochloride (30.0 mg, 0.063 mmol) was dissolved in DMA (1.00 mL) and (3R)-3-{4-[4-(2-oxopropyl)piperidin-1-yl]phenyl}piperidine-2,6-dione (20.7 mg, 0.063 mmol) was added in one portion. N,N-diisopropylethylamine (0.06 mL, 40.7 mg, 0.315 mmol) was then added followed by sodium triacetoxyborohydride (40.1 mg, 0.189 mmol). The resulting reaction was stirred at RT for 1-2 hrs and monitored via LCMS. Once complete, the reaction was concentrated in vacuo. The residue was dissolved in DMF and purified via RP-HPLC to yield the title compound (4.7 mg, 8.1%) as a white solid. LCMS: C₄₃H₅₇N₇O₅ requires: 751.4, found: m/z=752.3 [M+H]⁺

### Example 5

### (R)—N-CYCLOBUTYL-N-(1-(2-(1-(4-(2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-4-METHOXYPIPERIDINE-4-CARBOXAMIDE

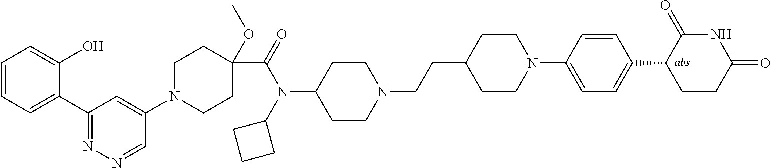 [SMILES: COC1(C(=O)N(C2CCC2)C2CCN(CCC3CCN(c4ccc([C@H]5CCC(=O)NC5=O)cc4)CC3)CC2)CCN(c2cnnc(-c3ccccc3O)c2)CC1]

### Step 1: Synthesis of N-cyclobutyl-1-(6-(2-hydroxyphenyl)pyridazin-4-yl)-4-methoxy-N-(piperidin-4-yl)piperidine-4-carboxamide hydrochloride

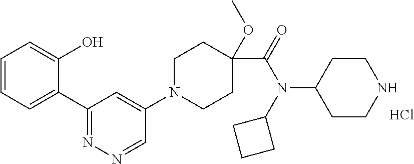 [SMILES: COC1(C(=O)N(C2CCC2)C2CCNCC2)CCN(c2cnnc(-c3ccccc3O)c2)CC1 ; Cl]

The title compound was prepared as described for Example 1 Step 1 using tert-butyl 4-(cyclobutylamino)piperidine-1-carboxylate (155 mg, 0.60 mmol) to afford the desired product as a yellow oil. LCMS C₃₃H₄₇N₅O₆ requires 465.3, found m/z 466.3 [M+H]⁺.

### Step 2: Synthesis of Title Compound

The title compound was prepared as described for Example 2, Step 2, using N-cyclobutyl-1-(6-(2-hydroxyphenyl)pyridazin-4-yl)-4-methoxy-N-(piperidin-4-yl)piperidine-4-carboxamide (32.6 mg, 0.07 mmol) to afford the title compound (13.1 mg, 22% yield) as a white solid. LCMS C₄₄H₅₇N₇O₅ requires 763.4, found m/z=764.4 [M+H]⁺.

### Example 6

### N-(1-(2-(1-(2-(2,6-DIOXOPIPERIDIN-3-YL)-1,3-DIOXOISOINDOLIN-5-YL)PIPERIDIN-4-YL)ETHYL)PIPERIDIN-4-YL)-1-(6-(2-HYDROXYPHENYL)PYRIDAZIN-4-YL)-4-METHOXY-N-METHYLPIPERIDINE-4-CARBOXAMIDE

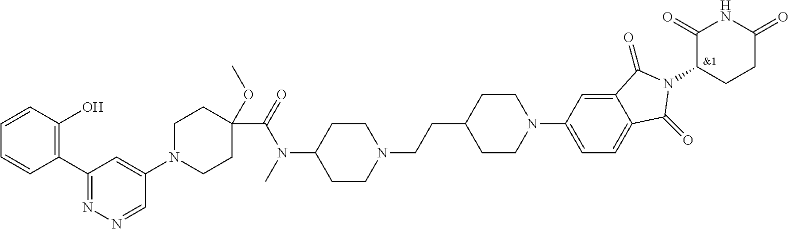 [SMILES: COC1(C(=O)N(C)C2CCN(CCC3CCN(c4ccc5c(c4)C(=O)N([C@H]4CCC(=O)NC4=O)C5=O)CC3)CC2)CCN(c2cnnc(-c3ccccc3O)c2)CC1]

### Step 1: Synthesis of 1-(6-(2-hydroxyphenyl)pyridazin-4-yl)-4-methoxy-N-methyl-N-(piperidin-4-yl)piperidine-4-carboxamide

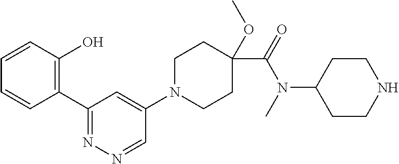 [SMILES: COC1(C(=O)N(C)C2CCNCC2)CCN(c2cnnc(-c3ccccc3O)c2)CC1]

To a solution of lithio 4-methoxy-1-{6-[2-(methoxymethoxy)phenyl]pyridazin-4-yl}piperidine-4-carboxylate (350.0 mg, 0.92 mmol) and tert-butyl 4-(methylamino)piperidine-1-carboxylate (247 μL, 247 mg, 1.15 mmol) in DMA (2.00 mL) was added N,N-diisopropylethylamine (483 μL, 0.36 g, 2.77 mmol). Then, [(dimethylamino)({[1,2,3]triazolo[4,5-b]pyridin-3-yloxy})methylidene]dimethylazanium; hexafluoro-lambda5-phosphanuide (526.24 mg, 1.38 mmol) was added and the reaction mixture was stirred at RT until complete by LCMS. Then, the solvent was removed under reduced pressure. The crude product was purified on silica gel with a gradient of EtOAc/Hex 0-100%, then 0-10% MeOH/DCM. Then, the pure material was dissolved in DCM (2.00 mL) and 3N HCl (1.00 mL) was added to the reaction mixture, and it was stirred at RT until complete by LCMS. Then, the solvent was removed under reduced pressure to yield the desired product. LCMS: C₂₃H₃₁N₅O₃ requires: 425.2, found: m/z=426.2 [M+H]⁺.

### Step 2: Synthesis of the Title Compound

The title compound was prepared as described for Example 1, Step 2, using 1-(6-(2-hydroxyphenyl)pyridazin-4-yl)-4-methoxy-N-methyl-N-(piperidin-4-yl)piperidine-4-carboxamide and 2-(1-(2-(2,6-dioxopiperidin-3-yl)-1,3-dioxoisoindolin-5-yl)piperidin-4-yl)acetaldehyde to afford rac-(R)—N-(1-(2-(1-(2-(2,6-dioxopiperidin-3-yl)-1,3-dioxoisoindolin-5-yl)piperidin-4-yl)ethyl)piperidin-4-yl)-1-(6-(2-hydroxyphenyl)pyridazin-4-yl)-4-methoxy-N-methylpiperidine-4-carboxamide (7.00 mg, 16% yield) as a yellow solid. LCMS: C₄₃H₅₂N₈O₇ requires 792.4, found: m/z=793.4 [M+H]⁺.

### Example 7

### N-(2-(DIMETHYLAMINO)ETHYL)-N-(1-((RS)-8-(4-((R)-2,6-DIOXOPIPERIDIN-3-YL)PHENYL)-1-OXA-8-AZASPIRO[4.5]DECAN-3-YL)PIPERIDIN-4-YL)-1-(6-(2-HYDROXYPHENYL)PYRIDAZIN-4-YL)-4-METHOXYPIPERIDINE-4-CARBOXAMIDE

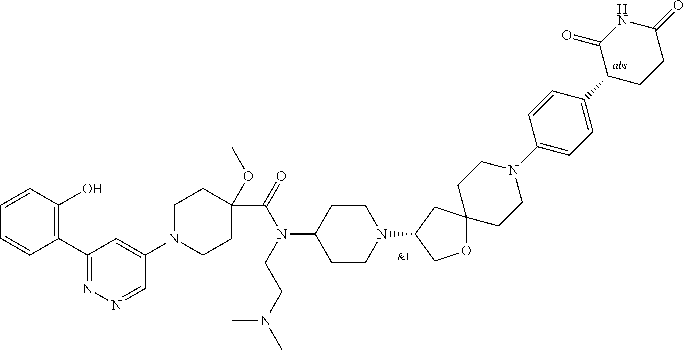 [SMILES: COC1(C(=O)N(CCN(C)C)C2CCN([C@H]3COC4(CCN(c5ccc([C@H]6CCC(=O)NC6=O)cc5)CC4)C3)CC2)CCN(c2cnnc(-c3ccccc3O)c2)CC1]

### Step 1: Synthesis of N-(2-(dimethylamino)ethyl)-1-(6-(2-hydroxyphenyl)pyridazin-4-yl)-4-methoxy-N-(piperidin-4-yl)piperidine-4-carboxamide

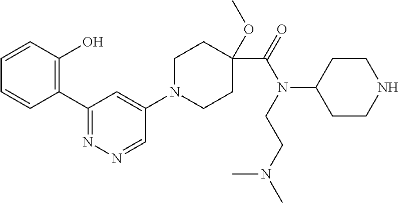 [SMILES: COC1(C(=O)N(CCN(C)C)C2CCNCC2)CCN(c2cnnc(-c3ccccc3O)c2)CC1]

N-(2-(dimethylamino)ethyl)-1-(6-(2-hydroxyphenyl)pyridazin-4-yl)-4-methoxy-N-(piperidin-4-yl)piperidine-4-carboxamide was prepared as described for Example 1, Step 1, using tert-butyl 4-((2-(dimethylamino)ethyl)amino)piperidine-1-carboxylate. LCMS: C₂₆H₃₈N₆O₃ requires: 482.3, found: m/z=483.4 [M+H]⁺.

### Step 2: Synthesis of the Title Compound

The title compound was prepared as described for Example 1, Step 2, using N-(2-(dimethylamino)ethyl)-1-(6-(2-hydroxyphenyl)pyridazin-4-yl)-4-methoxy-N-(piperidin-4-yl)piperidine-4-carboxamide and (R)-3-(4-(3-oxo-1-oxa-8-azaspiro[4.5]decan-8-yl)phenyl)piperidine-2,6-dione to afford N-(2-(dimethylamino)ethyl)-N-(1-((RS)-8-(4-((R)-2,6-dioxopiperidin-3-yl)phenyl)-1-oxa-8-azaspiro[4.5]decan-3-yl)piperidin-4-yl)-1-(6-(2-hydroxyphenyl)pyridazin-4-yl)-4-methoxypiperidine-4-carboxamide (10.20 mg, 24% yield) as a yellow solid. LCMS: C₄₅H₆₀N₈O₆ requires 780.4, found: m/z=781.1 [M+H]⁺.

### Example 8

### N-(1-((S)-8-(4-((R)-2,6-DIOXOPIPERIDIN-3-YL)PHENYL)-1-OXA-8-AZASPIRO[4.5]DECAN-3-YL)PIPERIDIN-4-YL)-N-ETHYL-1-(6-(2-HYDROXYPHENYL)PYRIDAZIN-4-YL)-4-METHOXYPIPERIDINE-4-CARBOXAMIDE

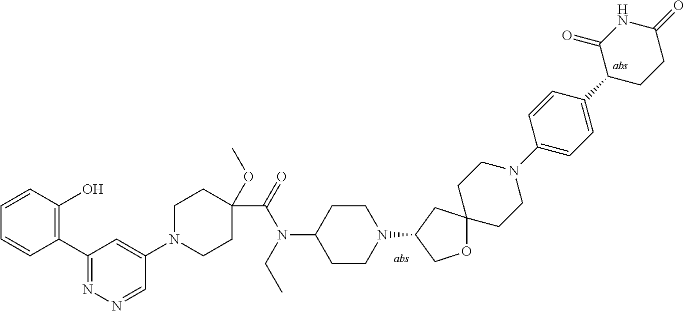 [SMILES: CCN(C(=O)C1(OC)CCN(c2cnnc(-c3ccccc3O)c2)CC1)C1CCN([C@H]2COC3(CCN(c4ccc([C@H]5CCC(=O)NC5=O)cc4)CC3)C2)CC1]

### Step 1: Synthesis of the Title Compound

N-ethyl-1-[6-(2-hydroxyphenyl)pyridazin-4-yl]-4-methoxy-N-(piperidin-4-yl)piperidine-4-carboxamide hydrochloride (200 mg, 0.421 mmol) was dissolved in DMA (1.00 mL) and (3S)-3-(4-{3-oxo-1-oxa-8-azaspiro[4.5]decan-8-yl}phenyl)piperidine-2,6-dione (172 mg, 0.504 mmol) was added in one portion. N,N-diisopropylethylamine (0.37 mL, 271 mg, 2.11 mmol) was then added followed by sodium triacetoxyborohydride (267 mg, 1.26 mmol). The resulting reaction was stirred at RT for 12 h and monitored via LCMS. Once complete, the reaction was concentrated in vacuo. The residue was dissolved in DMF and purified via RP-HPLC. The diastereomers were then separated via chiral SFC purification (RT=3.16 min, ChiralPak IH, MeOH+0.1% NH₄OH) to yield the product (44.8 mg, 13.9%) as a white solid. LCMS: C₄₃H₅₅N₇O₆ requires: 765.4, found: m/z=766.4 [M+H]⁺

### Example 9

### N-(1-((R)-8-(4-((R)-2,6-DIOXOPIPERIDIN-3-YL)PHENYL)-1-OXA-8-AZASPIRO[4.5]DECAN-3-YL)PIPERIDIN-4-YL)-N-ETHYL-1-(6-(2-HYDROXYPHENYL)PYRIDAZIN-4-YL)-4-METHOXYPIPERIDINE-4-CARBOXAMIDE

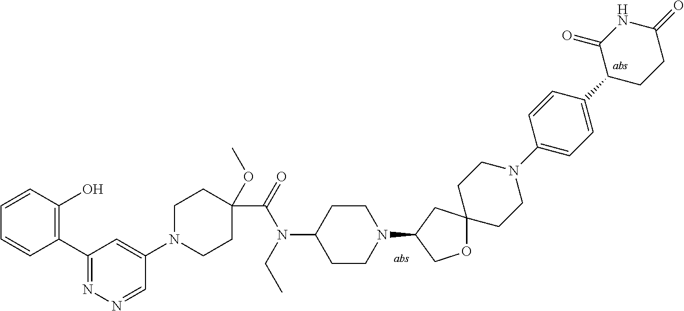 [SMILES: CCN(C(=O)C1(OC)CCN(c2cnnc(-c3ccccc3O)c2)CC1)C1CCN([C@@H]2COC3(CCN(c4ccc([C@H]5CCC(=O)NC5=O)cc4)CC3)C2)CC1]

N-ethyl-1-[6-(2-hydroxyphenyl)pyridazin-4-yl]-4-methoxy-N-(piperidin-4-yl)piperidine-4-carboxamide hydrochloride (200 mg, 0.421 mmol) was dissolved in DMA (1.00 mL) and (3S)-3-(4-{3-oxo-1-oxa-8-azaspiro[4.5]decan-8-yl}phenyl)piperidine-2,6-dione (172 mg, 0.504 mmol) was added in one portion. N,N-diisopropylethylamine (0.37 mL, 271 mg, 2.11 mmol) was then added followed by sodium triacetoxyborohydride (267 mg, 1.26 mmol). The resulting reaction was stirred at RT for 12 h and monitored via LCMS. Once complete, the reaction was concentrated in vacuo. The residue was dissolved in DMF and purified via RP-HPLC. The diastereomers were then separated via chiral SFC purification (RT=2.86 min, ChiralPak IH, MeOH+0.1% NH₄OH) to yield the product (44.9 mg, 13.9%) as a white solid. LCMS: C₄₃H₅₅N₇O₆ requires: 765.4, found: m/z=766.4 [M+H]⁺

### Example 10

### N-((1RS,5RS,6SR)-2-(2-(1-(4-((R)-2,6-DIOXOPIPERIDIN-3-YL)PHENYL)PIPERIDIN-4-YL)ETHYL)-2-AZABICYCLO[4.2.0]OCTAN-5-YL)-1-(6-(2-HYDROXYPHENYL)PYRIDAZIN-4-YL)-4-METHOXY-N-METHYLPIPERIDINE-4-CARBOXAMIDE

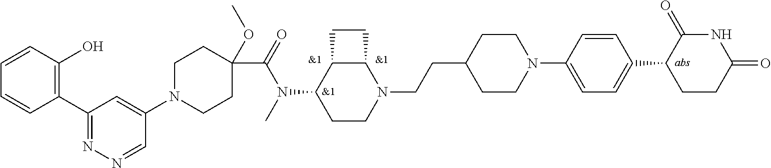 [SMILES: COC1(C(=O)N(C)[C@H]2CCN(CCC3CCN(c4ccc([C@H]5CCC(=O)NC5=O)cc4)CC3)[C@H]3CC[C@H]32)CCN(c2cnnc(-c3ccccc3O)c2)CC1]

### Step 1: Synthesis of rac-tert-butyl (1R,5R,6R)-5-(methylamino)-2-azabicyclo[4.2.0]octane-2-carboxylate

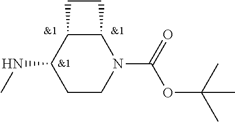 [SMILES: CN[C@H]1CCN(C(=O)OC(C)(C)C)[C@H]2CC[C@@H]12]

To a solution of rac-tert-butyl (1R,6S)-5-oxo-2-azabicyclo[4.2.0]octane-2-carboxylate (50.00 mg, 0.19 mmol) in DMA (0.50 mL) was added methylammonium chloride (52.89 mg, 0.78 mmol) and N,N-diisopropylethylamine (171.02 μL, 0.13 g, 0.98 mmol) and stirred for 30 min. Then, sodium triacetoxyborohydride (83.01 mg, 0.39 mmol) was added and stirred until complete by LCMS. The crude product was used in the next step without further purification. LCMS: C₁₃H₂₄N₂O₂ requires: 240.2, found: m/z=241.2 [M+H]⁺.

### Step 2: Synthesis of rac-N-((1R,5R,6S)-2-azabicyclo[4.2.0]octan-5-yl)-1-(6-(2-hydroxyphenyl)pyridazin-4-yl)-4-methoxy-N-methylpiperidine-4-carboxamide

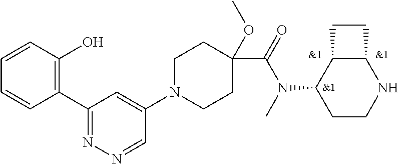 [SMILES: COC1(C(=O)N(C)[C@H]2CCN[C@H]3CC[C@H]32)CCN(c2cnnc(-c3ccccc3O)c2)CC1]

rac-N-((1R,5R,6S)-2-azabicyclo[4.2.0]octan-5-yl)-1-(6-(2-hydroxyphenyl)pyridazin-4-yl)-4-methoxy-N-methylpiperidine-4-carboxamide was prepared as described for Example 1, Step 1, using rac-tert-butyl (1R,5R,6R)-5-(methylamino)-2-azabicyclo[4.2.0]octane-2-carboxylate. LCMS: C₂₅H₃₃N₅O₃ requires: 451.2, found: m/z=452.4 [M+H]⁺.

### Step 3: Synthesis of the Title Compound

The title compound was prepared as described for Example 1, Step 2, using rac-N-((1R,5R,6S)-2-azabicyclo[4.2.0]octan-5-yl)-1-(6-(2-hydroxyphenyl)pyridazin-4-yl)-4-methoxy-N-methylpiperidine-4-carboxamide and (R)-2-(1-(4-(2,6-dioxopiperidin-3-yl)phenyl)piperidin-4-yl)acetaldehyde to afford N-((1RS,5RS,6SR)-2-(2-(1-(4-((R)-2,6-dioxopiperidin-3-yl)phenyl)piperidin-4-yl)ethyl)-2-azabicyclo[4.2.0]octan-5-yl)-1-(6-(2-hydroxyphenyl)pyridazin-4-yl)-4-methoxy-N-methylpiperidine-4-carboxamide (13.5 mg, 26% yield) as a white solid. LCMS: C₄₃H₅₅N₇O₅ requires 749.4, found: m/z=750.4 [M+H]⁺.

### Example 11

### (R)—N-(1-(2-(1-(4-(2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-N-ISOBUTYL-4-METHOXYPIPERIDINE-4-CARBOXAMIDE

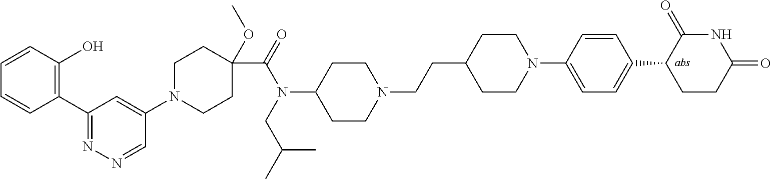 [SMILES: COC1(C(=O)N(CC(C)C)C2CCN(CCC3CCN(c4ccc([C@H]5CCC(=O)NC5=O)cc4)CC3)CC2)CCN(c2cnnc(-c3ccccc3O)c2)CC1]

### Step 1: Synthesis of the Title Compound

The title compound was prepared as described for Example 1, Step 2, using 1-(6-(2-hydroxyphenyl)pyridazin-4-yl)-N-isobutyl-4-methoxy-N-(piperidin-4-yl)piperidine-4-carboxamide and (R)-2-(1-(4-(2,6-dioxopiperidin-3-yl)phenyl)piperidin-4-yl)acetaldehyde to afford (R)—N-(1-(2-(1-(4-(2,6-dioxopiperidin-3-yl)phenyl)piperidin-4-yl)ethyl)piperidin-4-yl)-1-(6-(2-hydroxyphenyl)pyridazin-4-yl)-N-isobutyl-4-methoxypiperidine-4-carboxamide (22.4 mg, 44% yield) as white solid. LCMS: C₄₄H₅₉N₇O₅ requires 765.5, found: m/z=766.4 [M+H]⁺.

### Example 12

### N—((RS)-8-((1-(4-((R)-2,6-DIOXOPIPERIDIN-3-YL)PHENYL)PIPERIDIN-4-YL)METHYL)-1-OXA-8-AZASPIRO[4.5]DECAN-3-YL)-1-(6-(2-HYDROXYPHENYL)PYRIDAZIN-4-YL)-4-METHOXY-N-METHYLPIPERIDINE-4-CARBOXAMIDE

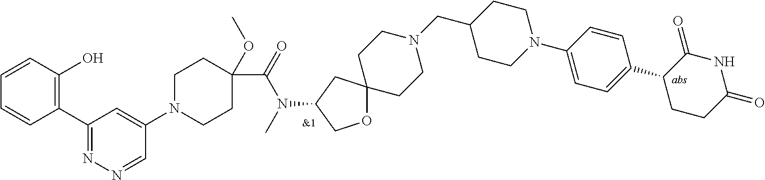 [SMILES: COC1(C(=O)N(C)[C@H]2COC3(CCN(CC4CCN(c5ccc([C@H]6CCC(=O)NC6=O)cc5)CC4)CC3)C2)CCN(c2cnnc(-c3ccccc3O)c2)CC1]

### Step 1: Synthesis of rac-(R)-1-(6-(2-hydroxyphenyl)pyridazin-4-yl)-4-methoxy-N-methyl-N-(1-oxa-8-azaspiro[4.5]decan-3-yl)piperidine-4-carboxamide

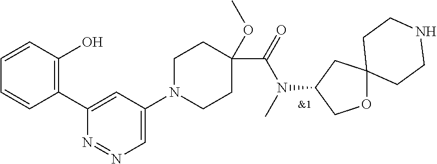 [SMILES: COC1(C(=O)N(C)[C@H]2COC3(CCNCC3)C2)CCN(c2cnnc(-c3ccccc3O)c2)CC1]

rac-(R)-1-(6-(2-hydroxyphenyl)pyridazin-4-yl)-4-methoxy-N-methyl-N-(1-oxa-8-azaspiro [4.5]decan-3-yl)piperidine-4-carboxamide was prepared as described for Example 1, Step 1, using rac-tert-butyl (R)-3-(methylamino)-1-oxa-8-azaspiro[4.5]decane-8-carboxylate. TFA in DCM was used for the cleavage of the Boc protecting group. LCMS: C₂₆H₃₅N₅O₄ requires: 481.2, found: m/z=482.4 [M+H]⁺.

### Step 2: Synthesis of the Title Compound

The title compound was prepared as described for Example 1, Step 2, using rac-(R)-1-(6-(2-hydroxyphenyl)pyridazin-4-yl)-4-methoxy-N-methyl-N-(1-oxa-8-azaspiro[4.5]decan-3-yl)piperidine-4-carboxamide and (R)-1-(4-(2,6-dioxopiperidin-3-yl)phenyl)piperidine-4-carbaldehyde to afford N—((RS)-8-((1-(4-((R)-2,6-dioxopiperidin-3-yl)phenyl)piperidin-4-yl)methyl)-1-oxa-8-azaspiro[4.5]decan-3-yl)-1-(6-(2-hydroxyphenyl)pyridazin-4-yl)-4-methoxy-N-methylpiperidine-4-carboxamide (29.4 mg, 68% yield) as an off-white solid. LCMS: C₄₃H₅₅N₇O₆ requires 765.4, found: m/z=766.4 [M+H]⁺.

### Example 13

### RAC-N-BENZYL-N-(1-((R)-8-(4-((S)-2,6-DIOXOPIPERIDIN-3-YL)PHENYL)-1-OXA-8-AZASPIRO[4.5]DECAN-3-YL)PIPERIDIN-4-YL)-1-(6-(2-HYDROXYPHENYL)PYRIDAZIN-4-YL)-4-METHOXYPIPERIDINE-4-CARBOXAMIDE

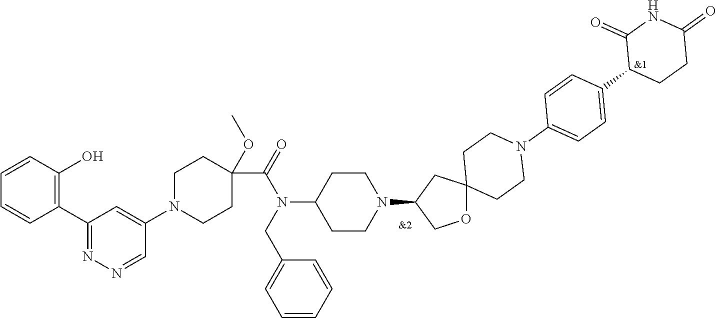 [SMILES: COC1(C(=O)N(Cc2ccccc2)C2CCN([C@@H]3COC4(CCN(c5ccc([C@H]6CCC(=O)NC6=O)cc5)CC4)C3)CC2)CCN(c2cnnc(-c3ccccc3O)c2)CC1]

### Step 1: Synthesis of N-benzyl-1-(6-(2-hydroxyphenyl)pyridazin-4-yl)-4-methoxy-N-(piperidin-4-yl)piperidine-4-carboxamide

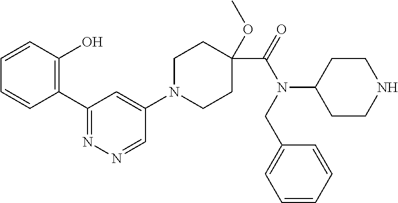 [SMILES: COC1(C(=O)N(Cc2ccccc2)C2CCNCC2)CCN(c2cnnc(-c3ccccc3O)c2)CC1]

To a solution of lithio 4-methoxy-1-{6-[2-(methoxymethoxy)phenyl]pyridazin-4-yl}piperidine-4-carboxylate (200 mg, 0.527 mmol) and tert-butyl 4-(benzylamino)piperidine-1-carboxylate (192 mg, 0.791 mmol) in DCM (5.00 mL) was added N,N-diisopropylethylamine (0.37 mL, 272 mg, 2.11 mmol). Then bis(2-oxo-1,3-oxazolidin-3-yl)phosphinoyl chloride (402 mg, 1.58 mmol) was added and the reaction mixture was stirred at 40° C. Then, the reaction was quenched with water, extracted with DCM 3×50 mL, dried, filtered and concentrated under reduced pressure. The crude material was purified via chromatography (MeOH:DCM, gradient) to yield the desired product as an orange foam. The material was dissolved in 1 mL methanol and 4M HCl in dioxane (1.0 mL) was added in one portion. The reaction was stirred at RT for 1 hour and then concentrated in vacuo to yield the desired product as a white solid. LCMS: C₂₉H₃₅N₅O₃ requires: 501.2, found: m/z=502.2 [M+H]⁺

### Step 2: Synthesis of the Title Compound

N-benzyl-1-[6-(2-hydroxyphenyl)pyridazin-4-yl]-4-methoxy-N-(piperidin-4-yl)piperidine-4-carboxamide hydrochloride (26.0 mg, 0.0483 mmol) was dissolved in DMA (1.00 mL) and (3S)-3-(4-{3-oxo-1-oxa-8-azaspiro[4.5]decan-8-yl}phenyl)piperidine-2,6-dione (16.5 mg, 0.0483 mmol) was added in one portion. N,N-diisopropylethylamine (31.23 mg, 0.241 mmol) was then added followed by sodium triacetoxyborohydride (30.7 mg, 0.145 mmol). The resulting reaction was stirred at RT overnight and monitored via LCMS. The resulting reaction was stirred at RT for 12 h and monitored via LCMS. Once complete, the reaction was concentrated in vacuo. The residue was dissolved in DMF and purified via RP-HPLC to yield the title compound (14.8 mg, 34.6%) as a white solid. LCMS: C₄₈H₅₇N₇O₆ requires: 827.4, found: m/z=828.4 [M+H]⁺

### Example 14

### RAC-N-(1-((R)-8-(4-((S)-2,6-DIOXOPIPERIDIN-3-YL)PHENYL)-1-OXA-8-AZASPIRO[4.5]DECAN-3-YL)PIPERIDIN-4-YL)-1-(6-(2-HYDROXYPHENYL)PYRIDAZIN-4-YL)-4-METHOXY-N-(2-METHOXYETHYL)PIPERIDINE-4-CARBOXAMIDE

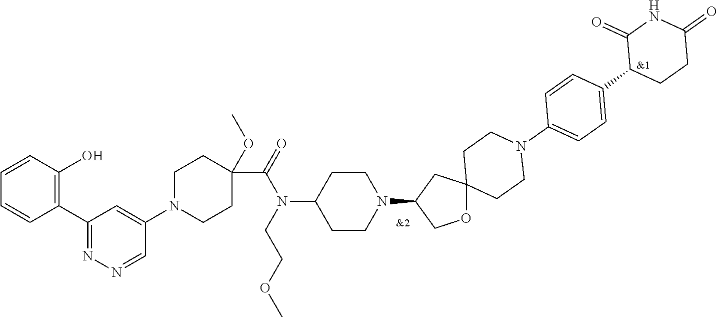 [SMILES: COCCN(C(=O)C1(OC)CCN(c2cnnc(-c3ccccc3O)c2)CC1)C1CCN([C@@H]2COC3(CCN(c4ccc([C@H]5CCC(=O)NC5=O)cc4)CC3)C2)CC1]

### Step 1: Synthesis of 1-(6-(2-hydroxyphenyl)pyridazin-4-yl)-4-methoxy-N-(2-methoxyethyl)-N-(piperidin-4-yl)piperidine-4-carboxamide

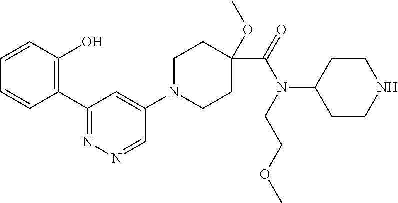 [SMILES: COCCN(C(=O)C1(OC)CCN(c2cnnc(-c3ccccc3O)c2)CC1)C1CCNCC1]

To a solution of lithio 4-methoxy-1-{6-[2-(methoxymethoxy)phenyl]pyridazin-4-yl}piperidine-4-carboxylate (200 mg, 0.527 mmol) and tert-butyl 4-[(2-methoxyethyl)amino]piperidine-1-carboxylate (191 mg, 0.791 mmol) in DCM (5.00 mL) was added N,N-diisopropylethylamine (0.37 mL, 272 mg, 2.11 mmol). Then bis(2-oxo-1,3-oxazolidin-3-yl)phosphinoyl chloride (402 mg, 1.58 mmol) was added and the reaction mixture was stirred at 40° C. Then, the reaction was quenched with water, extracted with DCM 3×50 mL, dried, filtered and concentrated under reduced pressure. The crude material was purified via chromatography (MeOH:DCM, gradient) to yield the desired product as an orange foam. The material was dissolved in 1 mL methanol and 4M HCl in dioxane (1.0 mL) was added in one portion. The reaction was stirred at RT for 1 hour and then concentrated in vacuo to yield the desired product as a white solid. LCMS: C₂₅H₃₅N₅O₄ requires: 469.2, found: m/z=470.3 [M+H]⁺

### Step 2: Synthesis of the Title Compound

1-[6-(2-hydroxyphenyl)pyridazin-4-yl]-4-methoxy-N-(2-methoxyethyl)-N-(piperidin-4-yl)piperidine-4-carboxamide hydrochloride (31.0 mg, 0.0613 mmol) was dissolved in DMA (1.00 mL) and (3S)-3-(4-{3-oxo-1-oxa-8-azaspiro[4.5]decan-8-yl}phenyl)piperidine-2,6-dione (20.9 mg, 0.0613 mmol) was added in one portion. N,N-diisopropylethylamine (39.6 mg, 0.306 mmol) was then added followed by sodium triacetoxyborohydride (38.9 mg, 0.183 mmol). The resulting reaction was stirred at RT overnight and monitored via LCMS. Once complete, the reaction was concentrated in vacuo. The residue was dissolved in DMF and purified via RP-HPLC to yield the title compound (25.3 mg, 47.3%) as a white solid. LCMS: C₄₄H₅₇N₇O₇ requires: 795.4, found: m/z=796.5 [M+H]⁺

### Example 15

### (R)—N-(1-(2-(1-(4-(2,6-DIOXOPIPERIDIN-3-YL)PHENYL)-4-FLUOROPIPERIDIN-4-YL)ETHYL)PIPERIDIN-4-YL)-N-ETHYL-1-(6-(2-HYDROXYPHENYL)PYRIDAZIN-4-YL)-4-METHOXYPIPERIDINE-4-CARBOXAMIDE

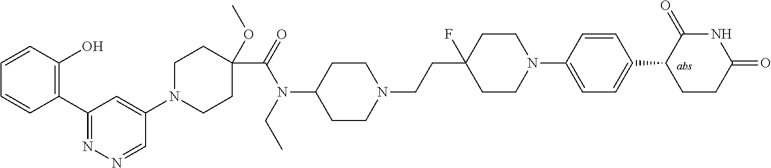 [SMILES: CCN(C(=O)C1(OC)CCN(c2cnnc(-c3ccccc3O)c2)CC1)C1CCN(CCC2(F)CCN(c3ccc([C@H]4CCC(=O)NC4=O)cc3)CC2)CC1]

### Step 1: Synthesis of the Title Compound

N-ethyl-1-[6-(2-hydroxyphenyl)pyridazin-4-yl]-4-methoxy-N-(piperidin-4-yl)piperidine-4-carboxamide hydrochloride (30.0 mg, 0.063 mmol) was dissolved in DMA (1.00 mL) and 2-(1-{4-[(3R)-2,6-dioxopiperidin-3-yl]phenyl}-4-fluoropiperidin-4-yl)acetaldehyde (20.7 mg, 0.063 mmol) was added in one portion. N,N-diisopropylethylamine (39.6 mg, 0.306 mmol) was then added followed by sodium triacetoxyborohydride (40.1 mg, 0.189 mmol). The resulting reaction was stirred at RT for 1-2 hrs and monitored via LCMS. Once complete, the reaction was concentrated in vacuo. The residue was dissolved in DMF and purified via RP-HPLC to yield the title compound (22.8 mg, 43.1%) as a tan solid. LCMS: C₄₂H₅₄FN₇O₅ requires: 755.4, found: m/z=756.5 [M+H]⁺

### Example 16

### RAC-(R)—N-(1-((1-(5-(2,6-DIOXOPIPERIDIN-3-YL)PYRIMIDIN-2-YL)PIPERIDIN-4-YL)METHYL)PIPERIDIN-4-YL)-N-ETHYL-1-(6-(2-HYDROXYPHENYL)PYRIDAZIN-4-YL)-4-METHOXYPIPERIDINE-4-CARBOXAMIDE

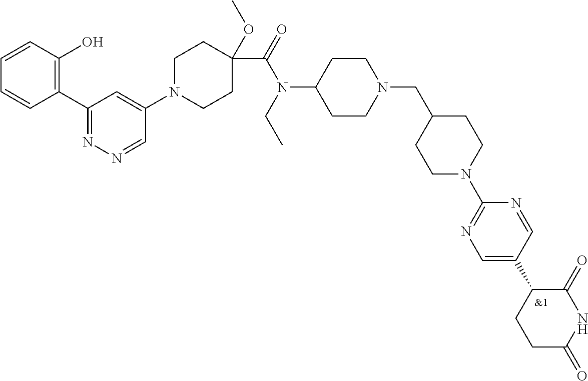 [SMILES: CCN(C(=O)C1(OC)CCN(c2cnnc(-c3ccccc3O)c2)CC1)C1CCN(CC2CCN(c3ncc([C@H]4CCC(=O)NC4=O)cn3)CC2)CC1]

### Step 1: Synthesis of the Title Compound

The title compound was prepared as described for Example 1, Step 2, using N-ethyl-1-(6-(2-hydroxyphenyl)pyridazin-4-yl)-4-methoxy-N-(piperidin-4-yl)piperidine-4-carboxamide and rac-(R)-1-(5-(2,6-dioxopiperidin-3-yl)pyrimidin-2-yl)piperidine-4-carbaldehyde to afford rac-(R)—N-(1-((1-(5-(2,6-dioxopiperidin-3-yl)pyrimidin-2-yl)piperidin-4-yl)methyl)piperidin-4-yl)-N-ethyl-1-(6-(2-hydroxyphenyl)pyridazin-4-yl)-4-methoxypiperidine-4-carboxamide (4.20 mg, 10% yield) as a white solid. LCMS: C₃₉H₅₁N₉O₅ requires 725.4, found: m/z=726.4 [M+H]⁺.

### Example 17

### (R)—N-(1-(2-(1-(4-(2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-4-METHOXY-N-(2-METHOXYETHYL)PIPERIDINE-4-CARBOXAMIDE

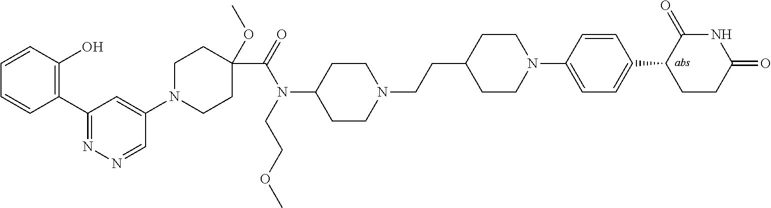 [SMILES: COCCN(C(=O)C1(OC)CCN(c2cnnc(-c3ccccc3O)c2)CC1)C1CCN(CCC2CCN(c3ccc([C@H]4CCC(=O)NC4=O)cc3)CC2)CC1]

### Step 1: Synthesis of the Title Compound

1-[6-(2-hydroxyphenyl)pyridazin-4-yl]-4-methoxy-N-(2-methoxyethyl)-N-(piperidin-4-yl)piperidine-4-carboxamide hydrochloride (31.0 mg, 0.061 mmol) was dissolved in DMA (1.00 mL) and 2-(1-{4-[(3R)-2,6-dioxopiperidin-3-yl]phenyl}piperidin-4-yl)acetaldehyde (23.1 mg, 0.073 mmol) was added in one portion. N,N-diisopropylethylamine (39.6 mg, 0.31 mmol) was then added followed by sodium triacetoxyborohydride (38.60 mg, 0.18 mmol). The resulting reaction was stirred at RT for 1-2 hrs and monitored via LCMS. Once complete, the reaction was concentrated in vacuo. The residue was dissolved in DMF and purified via RP-HPLC to yield the title compound (28.8 mg, 55.1%) as a yellow solid. LCMS: C₄₃H₅₇N₇O₆ requires: 767.4, found: m/z=767.4 [M+H]⁺

### Example 18

### RAC-(R)—N-(1-((1-(3-(2,6-DIOXOPIPERIDIN-3-YL)-4-FLUOROPHENYL)PIPERIDIN-4-YL)METHYL)PIPERIDIN-4-YL)-1-(6-(2-HYDROXYPHENYL)PYRIDAZIN-4-YL)-4-METHOXY-N-METHYLPIPERIDINE-4-CARBOXAMIDE

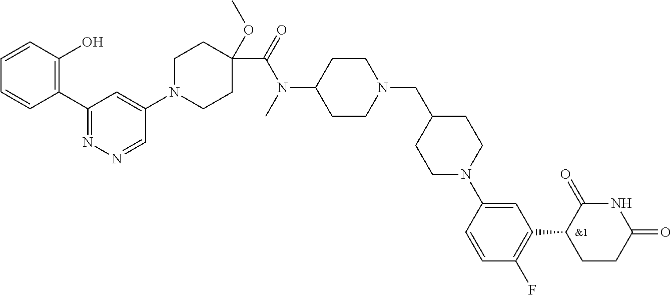 [SMILES: COC1(C(=O)N(C)C2CCN(CC3CCN(c4ccc(F)c([C@H]5CCC(=O)NC5=O)c4)CC3)CC2)CCN(c2cnnc(-c3ccccc3O)c2)CC1]

### Step 1: Synthesis of the Title Compound

The title compound was prepared as described for Example 1, Step 2, using rac-(R)-1-(3-(2,6-dioxopiperidin-3-yl)-4-fluorophenyl)piperidine-4-carbaldehyde and 1-(6-(2-hydroxyphenyl)pyridazin-4-yl)-4-methoxy-N-methyl-N-(piperidin-4-yl)piperidine-4-carboxamide to afford rac-(R)—N-(1-((1-(3-(2,6-dioxopiperidin-3-yl)-4-fluorophenyl)piperidin-4-yl)methyl)piperidin-4-yl)-1-(6-(2-hydroxyphenyl)pyridazin-4-yl)-4-methoxy-N-methylpiperidine-4-carboxamide (13.70 mg, 36% yield) as a white solid. LCMS: C₄₀H₅₀FN₇O₅ requires 727.4, found: m/z=728.3[M+H]⁺.

### Example 19

### N-(1-(((1R,4R)-4-(3-((RS)-2,6-DIOXOPIPERIDIN-3-YL)PHENOXY)CYCLOHEXYL)METHYL)PIPERIDIN-4-YL)-1-(6-(2-HYDROXYPHENYL)PYRIDAZIN-4-YL)-4-METHOXY-N-METHYLPIPERIDINE-4-CARBOXAMIDE

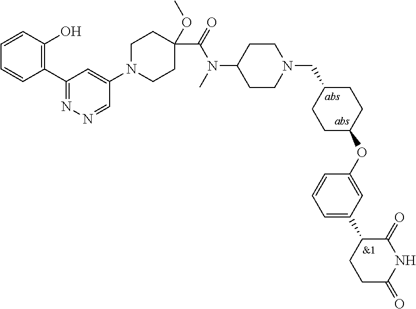 [SMILES: COC1(C(=O)N(C)C2CCN(C[C@H]3CC[C@H](Oc4cccc([C@H]5CCC(=O)NC5=O)c4)CC3)CC2)CCN(c2cnnc(-c3ccccc3O)c2)CC1]

### Step 1: Synthesis of the Title Compound

The title compound was prepared as described for Example 1, Step 2, using (1R,4r)-4-(3-((RS)-2,6-dioxopiperidin-3-yl)phenoxy)cyclohexane-1-carbaldehyde and 1-(6-(2-hydroxyphenyl)pyridazin-4-yl)-4-methoxy-N-methyl-N-(piperidin-4-yl)piperidine-4-carboxamide to afford N-(1-(((1R,4r)-4-(3-((RS)-2,6-dioxopiperidin-3-yl)phenoxy)cyclohexyl)methyl)piperidin-4-yl)-1-(6-(2-hydroxyphenyl)pyridazin-4-yl)-4-methoxy-N-methylpiperidine-4-carboxamide (10.70 mg, 29% yield) as a white solid. LCMS: C₄₁H₅₂N₆O₆ requires 724.4, found: m/z=725.4[M+H]⁺.

### Example 20

### (R)—N-(1-(2-(1-(4-(2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-N,4-DIMETHOXYPIPERIDINE-4-CARBOXAMIDE

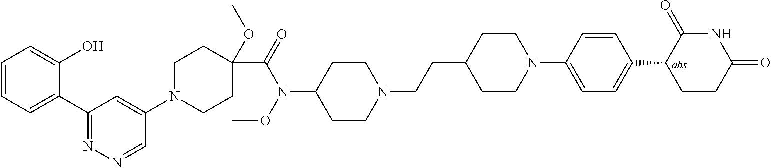 [SMILES: CON(C(=O)C1(OC)CCN(c2cnnc(-c3ccccc3O)c2)CC1)C1CCN(CCC2CCN(c3ccc([C@H]4CCC(=O)NC4=O)cc3)CC2)CC1]

### Step 1: Synthesis of 1-(6-(2-hydroxyphenyl)pyridazin-4-yl)-N,4-dimethoxy-N-(piperidin-4-yl)piperidine-4-carboxamide hydrochloride

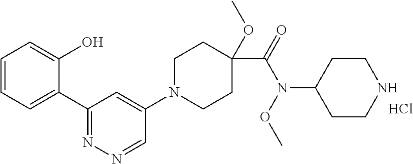 [SMILES: CON(C(=O)C1(OC)CCN(c2cnnc(-c3ccccc3O)c2)CC1)C1CCNCC1 ; Cl]

This compound was prepared as described for Example 1 Step 1, using tert-butyl 4-(methoxyamino)piperidine-1-carboxylate (91.4 mg, 0.40 mmol) to afford the desired compound as a yellow oil. LCMS C₂₃H₃₁N₅O₄ requires 441.3 found m/z=442.3 [M+H]⁺.

### Step 3: Synthesis of the Title Compound

The title compound was prepared as described for the synthesis of Example 2 Step 2, using 1-(6-(2-hydroxyphenyl)pyridazin-4-yl)-N,4-dimethoxy-N-(piperidin-4-yl)piperidine-4-carboxamide hydrochloride (21.6 mg, 0.05 mmol) to afford the title compound (2.50 mg, 6% yield) as a white solid. LCMS C₄₁H₅₃N₇O₆ requires 739.4, found m/z=740.4 [M+H]⁺.

### Example 21

### (R)-3-(4-((RS)-3-(8-(1-(6-(2-HYDROXYPHENYL)PYRIDAZIN-4-YL)-4-METHOXYPIPERIDINE-4-CARBONYL)-2,8-DIAZASPIRO[4.5]DECAN-2-YL)-1-OXA-8-AZASPIRO[4.5]DECAN-8-YL)PHENYL)PIPERIDINE-2,6-DIONE

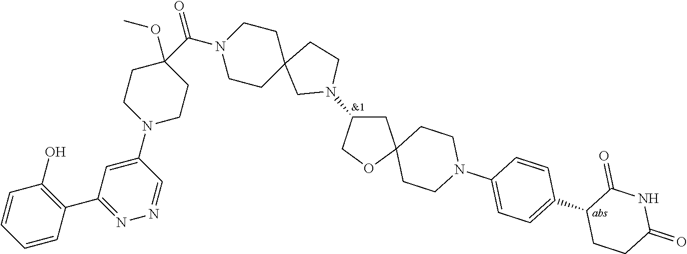 [SMILES: COC1(C(=O)N2CCC3(CC2)CCN([C@H]2COC4(CCN(c5ccc([C@H]6CCC(=O)NC6=O)cc5)CC4)C2)C3)CCN(c2cnnc(-c3ccccc3O)c2)CC1]

### Step 1: Synthesis of (1-(6-(2-hydroxyphenyl)pyridazin-4-yl)-4-methoxypiperidin-4-yl)(2,8-diazaspiro[4.5]decan-8-yl)methanone

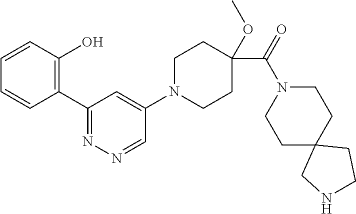 [SMILES: COC1(C(=O)N2CCC3(CCNC3)CC2)CCN(c2cnnc(-c3ccccc3O)c2)CC1]

(1-(6-(2-hydroxyphenyl)pyridazin-4-yl)-4-methoxypiperidin-4-yl)(2,8-diazaspiro[4.5]decan-8-yl)methanone was prepared as described for Example 1, Step 1, tert-butyl 2,8-diazaspiro[4.5]decane-2-carboxylate. LCMS: C₂₅H₃₃N₅O₃ requires: 451.2, found: m/z=452.4 [M+H]⁺.

### Step 2: Synthesis of the Title Compound

The title compound was prepared as described for Example 1, Step 2, using (1-(6-(2-hydroxyphenyl)pyridazin-4-yl)-4-methoxypiperidin-4-yl)(2,8-diazaspiro[4.5]decan-8-yl)methanone and (R)-3-(4-(3-oxo-1-oxa-8-azaspiro[4.5]decan-8-yl)phenyl)piperidine-2,6-dione to afford (R)-3-(4-((RS)-3-(8-(1-(6-(2-hydroxyphenyl)pyridazin-4-yl)-4-methoxypiperidine-4-carbonyl)-2,8-diazaspiro[4.5]decan-2-yl)-1-oxa-8-azaspiro[4.5]decan-8-yl)phenyl)piperidine-2,6-dione (11.30 mg, 22% yield) as a white solid. LCMS: C₄₁H₅₅N₇O₆ requires 777.4, found: m/z=778.4 [M+H]⁺.

### Example 22

### N-(1-((1-(4-(2,4-DIOXOTETRAHYDROPYRIMIDIN-1(2H)-YL)PHENYL)PIPERIDIN-4-YL)METHYL)PIPERIDIN-4-YL)-N-ETHYL-1-(6-(2-HYDROXYPHENYL)PYRIDAZIN-4-YL)-4-METHOXYPIPERIDINE-4-CARBOXAMIDE

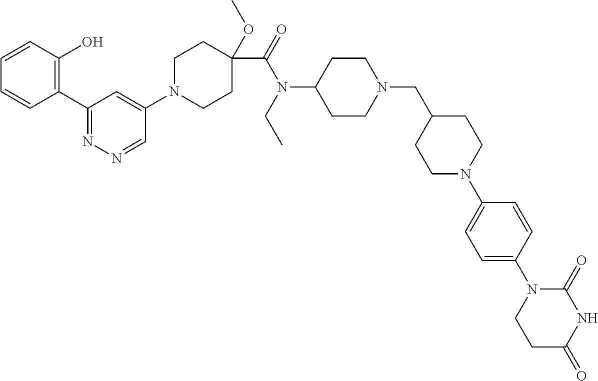 [SMILES: CCN(C(=O)C1(OC)CCN(c2cnnc(-c3ccccc3O)c2)CC1)C1CCN(CC2CCN(c3ccc(N4CCC(=O)NC4=O)cc3)CC2)CC1]

### Step 1: Synthesis of the Title Compound

The title compound was prepared as described for Example 1, Step 2, using 1-(4-(2,4-dioxotetrahydropyrimidin-1(2H)-yl)phenyl)piperidine-4-carbaldehyde and N-ethyl-1-(6-(2-hydroxyphenyl)pyridazin-4-yl)-4-methoxy-N-(piperidin-4-yl)piperidine-4-carboxamide to afford N-(1-((1-(4-(2,4-dioxotetrahydropyrimidin-1(2H)-yl)phenyl)piperidin-4-yl)methyl)piperidin-4-yl)-N-ethyl-1-(6-(2-hydroxyphenyl)pyridazin-4-yl)-4-methoxypiperidine-4-carboxamide (11.80 mg, 33% yield) as a white solid. LCMS: C₄₀H₅₂N₈O₅ requires 724.4, found: m/z=725.4 [M+H]⁺.

### Example 23

### RAC-(R)—N-(1-(2-(4-(4-(2,6-DIOXOPIPERIDIN-3-YL)PHENYL)-1-OXA-4,9-DIAZASPIRO[5.5]UNDECAN-9-YL)ACETYL)PIPERIDIN-4-YL)-N-ETHYL-1-(6-(2-HYDROXYPHENYL)PYRIDAZIN-4-YL)-4-METHOXYPIPERIDINE-4-CARBOXAMIDE

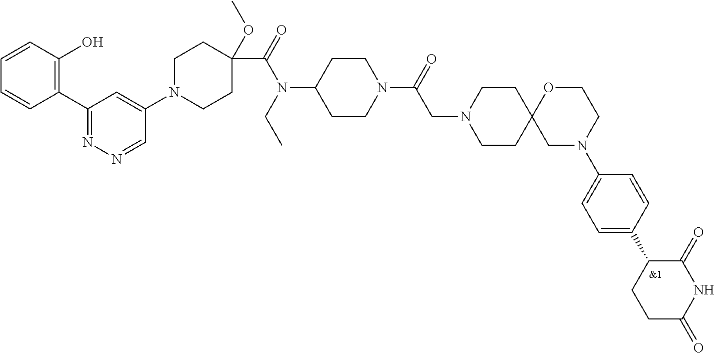 [SMILES: CCN(C(=O)C1(OC)CCN(c2cnnc(-c3ccccc3O)c2)CC1)C1CCN(C(=O)CN2CCC3(CC2)CN(c2ccc([C@H]4CCC(=O)NC4=O)cc2)CCO3)CC1]

### Step 1: Synthesis of the Title Compound

To a solution of N-ethyl-1-[6-(2-hydroxyphenyl)pyridazin-4-yl]-4-methoxy-N-(piperidin-4-yl)piperidine-4-carboxamide (20.01 mg, 0.05 mmol) and rac-(4-{4-[(3R)-2,6-dioxopiperidin-3-yl]phenyl}-1-oxa-4,9-diazaspiro[5.5]undecan-9-yl)acetic acid hydrochloride (25.92 mg, 0.06 mmol) in DMA (0.50 mL), was added N,N-diisopropylethylamine (39.75 μL, 0.03 g, 0.23 mmol) followed by [(dimethylamino)({[1,2,3]triazolo[4,5-b]pyridin-3-yloxy})methylidene]dimethylazanium; hexafluoro-lambda5-phosphanuide (20.77 mg, 0.05 mmol) and the reaction mixture was stirred at RT until complete by LCMS. Once complete, the solution was injected onto RP—FC and purified with a gradient of 5-95% MeCN in H₂O to yield the title compound (10.50 mg, 23% yield) as a gray solid. LCMS: C₄₅H₅₈N₈O₇ requires 822.4, found: m/z=823.4[M+H]⁺.

### Example 24

### (R)-3-(4-(4-(2-((3ARS,7ASR)-1-(1-(6-(2-HYDROXYPHENYL)PYRIDAZIN-4-YL)-4-METHOXYPIPERIDINE-4-CARBONYL)OCTAHYDRO-5H-PYRROLO[3,2-C]PYRIDIN-5-YL)ETHYL)PIPERIDIN-1-YL)PHENYL)PIPERIDINE-2,6-DIONE

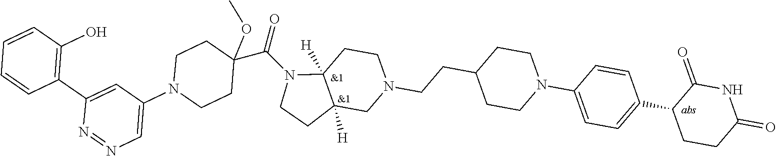 [SMILES: COC1(C(=O)N2CC[C@@H]3CN(CCC4CCN(c5ccc([C@H]6CCC(=O)NC6=O)cc5)CC4)CC[C@@H]32)CCN(c2cnnc(-c3ccccc3O)c2)CC1]

### Step 1: Synthesis of rac-(1-(6-(2-hydroxyphenyl)pyridazin-4-yl)-4-methoxypiperidin-4-yl)((3aR,7aS)-octahydro-1H-pyrrolo[3,2-c]pyridin-1-yl)methanone hydrochloride

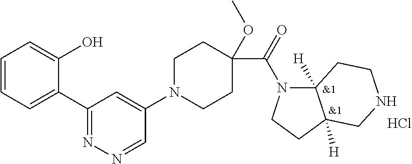 [SMILES: COC1(C(=O)N2CC[C@@H]3CNCC[C@@H]32)CCN(c2cnnc(-c3ccccc3O)c2)CC1 ; Cl]

This compound was prepare as described or Example 1 Step 1 using rac-tert-butyl (3aS,7aR)-octahydro-5H-pyrrolo[3,2-c]pyridine-5-carboxylate (32.1 mg, 0.08 mmol) to afford the desired product as an orange solid. LCMS C₂₄H₃₁N₅O₃ requires 437.2, found 438.3 [M+H]⁺.

### Step 3: Synthesis of Title Compound

The title compound was prepared as described for Example 2 Step 2, using rac-(1-(6-(2-hydroxyphenyl)pyridazin-4-yl)-4-methoxypiperidin-4-yl)((3aR,7aS)-octahydro-1H-pyrrolo[3,2-c]pyridin-1-yl)methanone (35.2 mg, 0.08 mmol) to afford the desired product (36.6 mg, 50% yield) as a bright orange solid. LCMS C₄₂H₅₃N₇O₅ requires 735.4, found m/z=736.4 [M+H]⁺.

### Example 25

### (R)-3-(4-((RS)-3-((3ARS,7ASR)-1-(1-(6-(2-HYDROXYPHENYL)PYRIDAZIN-4-YL)-4-METHOXYPIPERIDINE-4-CARBONYL)OCTAHYDRO-5H-PYRROLO[3,2-C]PYRIDIN-5-YL)-1-OXA-8-AZASPIRO[4.5]DECAN-8-YL)PHENYL)PIPERIDINE-2,6-DIONE

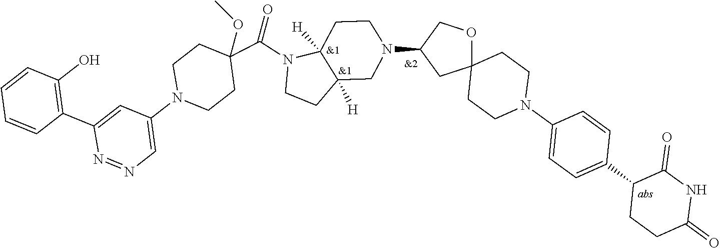 [SMILES: COC1(C(=O)N2CC[C@@H]3CN([C@H]4COC5(CCN(c6ccc([C@H]7CCC(=O)NC7=O)cc6)CC5)C4)CC[C@@H]32)CCN(c2cnnc(-c3ccccc3O)c2)CC1]

### Step 1: Synthesis of Title Compound

The title compound was prepared as described for Example 2 Step 2, using rac-(1-(6-(2-hydroxyphenyl)pyridazin-4-yl)-4-methoxypiperidin-4-yl)((3aR,7aS)-octahydro-1H-pyrrolo[3,2-c]pyridin-1-yl)methanone hydrochloride (25.3 mg, 0.06 mmol) and (R)-3-(4-(3-oxo-1-oxa-8-azaspiro[4.5]decan-8-yl)phenyl)piperidine-2,6-dione to afford the title compound (40.9 mg, 80% yield) as a white solid. LCMS C₄₃H₅₃N₇O₆ requires 763.4 found m/z=764.4 [M+H]⁺.

### Example 26

### RAC-N-(1-((R)-8-(4-((S)-2,6-DIOXOPIPERIDIN-3-YL)-3-FLUOROPHENYL)-1-OXA-8-AZASPIRO[4.5]DECAN-3-YL)PIPERIDIN-4-YL)-N-ETHYL-1-(6-(2-HYDROXYPHENYL)PYRIDAZIN-4-YL)-4-METHOXYPIPERIDINE-4-CARBOXAMIDE

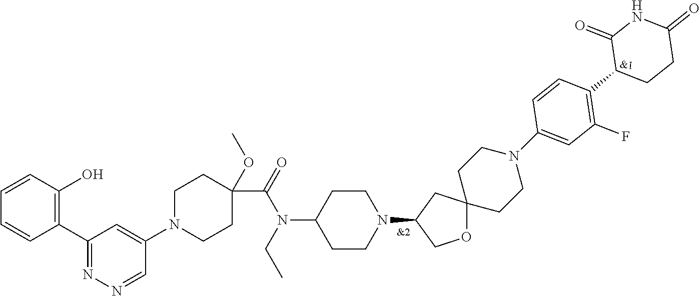 [SMILES: CCN(C(=O)C1(OC)CCN(c2cnnc(-c3ccccc3O)c2)CC1)C1CCN([C@@H]2COC3(CCN(c4ccc([C@H]5CCC(=O)NC5=O)c(F)c4)CC3)C2)CC1]

### Step 1: Synthesis of the Title Compound

N-ethyl-1-[6-(2-hydroxyphenyl)pyridazin-4-yl]-4-methoxy-N-(piperidin-4-yl)piperidine-4-carboxamide hydrochloride (30.0 mg, 0.063 mmol) was dissolved in DMA (1.00 mL) and (3S)-3-(2-fluoro-4-{3-oxo-1-oxa-8-azaspiro[4.5]decan-8-yl}phenyl)piperidine-2,6-dione (22.7 mg, 0.063 mmol) was added in one portion. N,N-diisopropylethylamine (40.7 mg, 0.315 mmol) was then added followed by sodium triacetoxyborohydride (40.1 mg, 0.189 mmol). The resulting reaction was stirred at RT overnight and monitored via LCMS. Once complete, the reaction was concentrated in vacuo. The residue was dissolved in DMF and purified via RP-HPLC to yield the title compound (23.3 mg, 44.1%) as a tan solid. LCMS: C₄₃H₅₄FN₇O₆ requires: 783.4, found: m/z=783.4 [M+H]⁺

### Example 27

### RAC-N-(1-((R)-8-(4-((S)-2,6-DIOXOPIPERIDIN-3-YL)-3,5-DIFLUOROPHENYL)-1-OXA-8-AZASPIRO[4.5]DECAN-3-YL)PIPERIDIN-4-YL)-N-ETHYL-1-(6-(2-HYDROXYPHENYL)PYRIDAZIN-4-YL)-4-METHOXYPIPERIDINE-4-CARBOXAMIDE

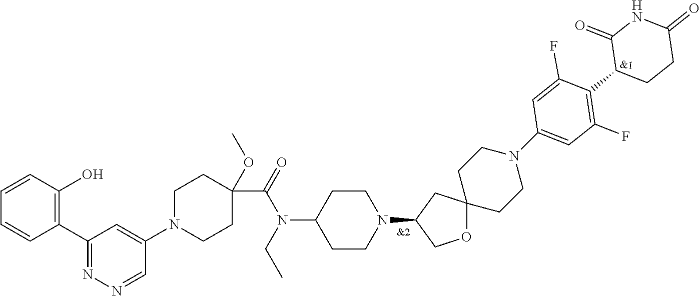 [SMILES: CCN(C(=O)C1(OC)CCN(c2cnnc(-c3ccccc3O)c2)CC1)C1CCN([C@@H]2COC3(CCN(c4cc(F)c([C@H]5CCC(=O)NC5=O)c(F)c4)CC3)C2)CC1]

### Step 1: Synthesis of the Title Compound

N-ethyl-1-[6-(2-hydroxyphenyl)pyridazin-4-yl]-4-methoxy-N-(piperidin-4-yl)piperidine-4-carboxamide hydrochloride (30.0 mg, 0.063 mmol) was dissolved in DMA (1.00 mL) and (3S)-3-(2,6-difluoro-4-{3-oxo-1-oxa-8-azaspiro[4.5]decan-8-yl}phenyl)piperidine-2,6-dione (23.8 mg, 0.063 mmol) was added in one portion. N,N-diisopropylethylamine (40.7 mg, 0.315 mmol) was then added followed by sodium triacetoxyborohydride (40.1 mg, 0.189 mmol). The resulting reaction was stirred at RT overnight and monitored via LCMS. Once complete, the reaction was concentrated in vacuo. The residue was dissolved in DMF and purified via RP-HPLC to yield the title compound (25.7 mg, 48.1%) as a white solid. LCMS: C₄₃H₅₃F₂N₇O₆ requires: 801.4, found: m/z=801.5 [M+H]⁺

### Example 28

### RAC-(R)—N-(1-(2-(1-(5-(2,6-DIOXOPIPERIDIN-3-YL)PYRIDIN-2-YL)PIPERIDIN-4-YL)ETHYL)PIPERIDIN-4-YL)-N-ETHYL-1-(6-(2-HYDROXYPHENYL)PYRIDAZIN-4-YL)-4-METHOXYPIPERIDINE-4-CARBOXAMIDE

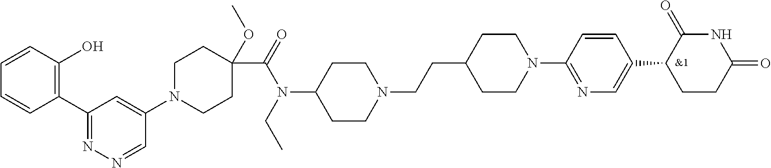 [SMILES: CCN(C(=O)C1(OC)CCN(c2cnnc(-c3ccccc3O)c2)CC1)C1CCN(CCC2CCN(c3ccc([C@H]4CCC(=O)NC4=O)cn3)CC2)CC1]

### Step 1: Synthesis of the Title Compound

The title compound was prepared as described for Example 1, Step 2, using N-ethyl-1-(6-(2-hydroxyphenyl)pyridazin-4-yl)-4-methoxy-N-(piperidin-4-yl)piperidine-4-carboxamide and rac-(R)-2-(1-(5-(2,6-dioxopiperidin-3-yl)pyridin-2-yl)piperidin-4-yl)acetaldehyde to afford rac-(R)—N-(1-(2-(1-(5-(2,6-dioxopiperidin-3-yl)pyridin-2-yl)piperidin-4-yl)ethyl)piperidin-4-yl)-N-ethyl-1-(6-(2-hydroxyphenyl)pyridazin-4-yl)-4-methoxypiperidine-4-carboxamide (18.8 mg, 27% yield). LCMS: C₄₁H₅₄N₈O₅ requires 738.4, found: m/z=739.4 [M+H]⁺.

### Example 29

### RAC-N-(1-((R)-8-(4-((R)-2,6-DIOXOPIPERIDIN-3-YL)PHENYL)-8-AZASPIRO[4.5]DECAN-2-YL)PIPERIDIN-4-YL)-N-ETHYL-1-(6-(2-HYDROXYPHENYL)PYRIDAZIN-4-YL)-4-METHOXYPIPERIDINE-4-CARBOXAMIDE

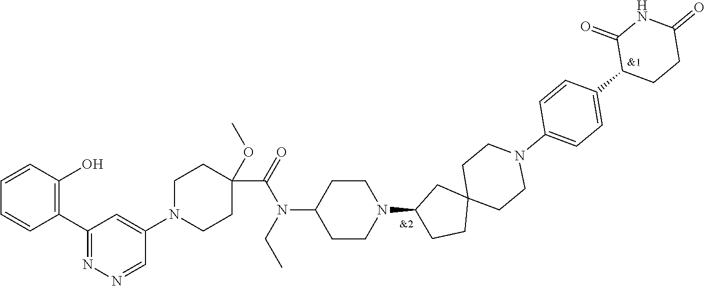 [SMILES: CCN(C(=O)C1(OC)CCN(c2cnnc(-c3ccccc3O)c2)CC1)C1CCN([C@@H]2CCC3(CCN(c4ccc([C@H]5CCC(=O)NC5=O)cc4)CC3)C2)CC1]

### Step 1: Synthesis of the Title Compound

N-ethyl-1-[6-(2-hydroxyphenyl)pyridazin-4-yl]-4-methoxy-N-(piperidin-4-yl)piperidine-4-carboxamide hydrochloride (30.0 mg, 0.063 mmol) was dissolved in DMA (1.00 mL) and (3S)-3-(4-{2-oxo-8-azaspiro[4.5]decan-8-yl}phenyl)piperidine-2,6-dione (21.4 mg, 0.063 mmol) was added in one portion. N,N-diisopropylethylamine (40.7 mg, 0.315 mmol) was then added followed by sodium triacetoxyborohydride (40.1 mg, 0.189 mmol). The resulting reaction was stirred at RT overnight and monitored via LCMS. Once complete, the reaction was concentrated in vacuo. The residue was dissolved in DMF and purified via RP-HPLC to yield the title compound (25.0 mg, 50.4%) as a yellow solid. LCMS: C₄₄H₅₇N₇O₅ requires: 763.4, found: m/z=764.2 [M+H]⁺

### Example 30

### (R)—N-(1-(2-(1-(4-(2,6-DIOXOPIPERIDIN-3-YL)-3-FLUOROPHENYL)PIPERIDIN-4-YL)ETHYL)PIPERIDIN-4-YL)-N-ETHYL-1-(6-(2-HYDROXYPHENYL)PYRIDAZIN-4-YL)-4-METHOXYPIPERIDINE-4-CARBOXAMIDE

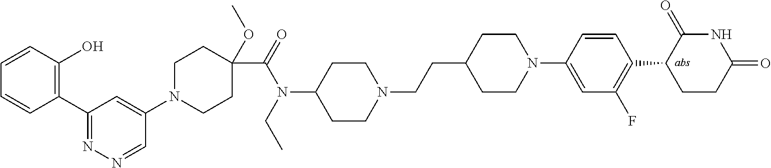 [SMILES: CCN(C(=O)C1(OC)CCN(c2cnnc(-c3ccccc3O)c2)CC1)C1CCN(CCC2CCN(c3ccc([C@H]4CCC(=O)NC4=O)c(F)c3)CC2)CC1]

### Step 1: Synthesis of the Title Compound

N-ethyl-1-[6-(2-hydroxyphenyl)pyridazin-4-yl]-4-methoxy-N-(piperidin-4-yl)piperidine-4-carboxamide hydrochloride (30.0 mg, 0.063 mmol) was dissolved in DMA (1.00 mL) and 2-(1-{4-[(3R)-2,6-dioxopiperidin-3-yl]-3-fluorophenyl}piperidin-4-yl)acetaldehyde (20.9 mg, 0.063 mmol) was added in one portion. N,N-diisopropylethylamine (0.06 mL, 40.7 mg, 0.315 mmol) was then added followed by sodium triacetoxyborohydride (40.1 mg, 0.189 mmol). The resulting reaction was stirred at RT for 1-2 hrs and monitored via LCMS. Once complete, the reaction was concentrated in vacuo. The residue was dissolved in DMF and purified via RP-HPLC to yield the title compound (26.3 mg, 54.3%) as a tan solid. LCMS: C₄₂H₅₄FN₇O₅ requires: 755.4, found: m/z=756.4 [M+H]⁺

### Example 31

### (R)—N-(1-(2-(1-(4-(2,6-DIOXOPIPERIDIN-3-YL)-3,5-DIFLUOROPHENYL)PIPERIDIN-4-YL)ETHYL)PIPERIDIN-4-YL)-N-ETHYL-1-(6-(2-HYDROXYPHENYL)PYRIDAZIN-4-YL)-4-METHOXYPIPERIDINE-4-CARBOXAMIDE

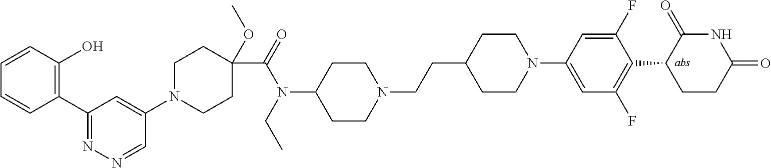 [SMILES: CCN(C(=O)C1(OC)CCN(c2cnnc(-c3ccccc3O)c2)CC1)C1CCN(CCC2CCN(c3cc(F)c([C@H]4CCC(=O)NC4=O)c(F)c3)CC2)CC1]

### Step 1: Synthesis of the Title Compound

N-ethyl-1-[6-(2-hydroxyphenyl)pyridazin-4-yl]-4-methoxy-N-(piperidin-4-yl)piperidine-4-carboxamide hydrochloride (30.0 mg, 0.063 mmol) was dissolved in DMA (1.00 mL) and 2-(1-{4-[(3R)-2,6-dioxopiperidin-3-yl]-3,5-difluorophenyl}piperidin-4-yl)acetaldehyde (22.1 mg, 0.063 mmol) was added in one portion. N,N-diisopropylethylamine (40.7 mg, 0.315 mmol) was then added followed by sodium triacetoxyborohydride (40.1 mg, 0.189 mmol). The resulting reaction was stirred at RT for 1-2 hrs and monitored via LCMS. Once complete, the reaction was concentrated in vacuo. The residue was dissolved in DMF and purified via RP-HPLC to yield the title compound (28.6 mg, 58.6%) as a white solid. LCMS: C₄₂H₅₃F₂N₇O₅ requires: 773.4, found: m/z=774.6 [M+H]⁺

### Example 32

### RAC-N-(1-((R)-8-(4-((S)-2,6-DIOXOPIPERIDIN-3-YL)PHENYL)-1-OXA-8-AZASPIRO[4.5]DECAN-3-YL)PIPERIDIN-4-YL)-1-(6-(5-FLUORO-2-HYDROXYPHENYL)PYRIDAZIN-4-YL)-4-METHOXY-N-METHYLPIPERIDINE-4-CARBOXAMIDE

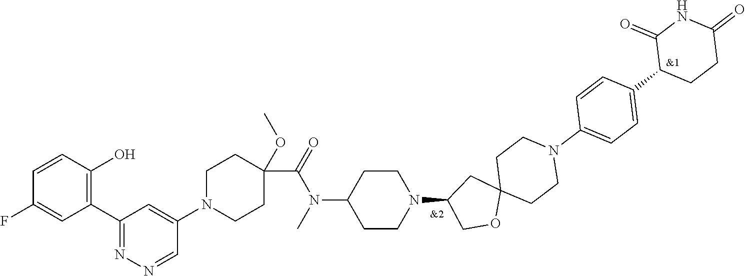 [SMILES: COC1(C(=O)N(C)C2CCN([C@@H]3COC4(CCN(c5ccc([C@H]6CCC(=O)NC6=O)cc5)CC4)C3)CC2)CCN(c2cnnc(-c3cc(F)ccc3O)c2)CC1]

### Step 1: Synthesis of 1-(6-(5-fluoro-2-hydroxyphenyl)pyridazin-4-yl)-4-methoxy-N-methyl-N-(piperidin-4-yl)piperidine-4-carboxamide

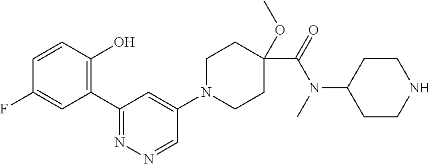 [SMILES: COC1(C(=O)N(C)C2CCNCC2)CCN(c2cnnc(-c3cc(F)ccc3O)c2)CC1]

dilithium(1+) 1-[6-(5-fluoro-2-oxidophenyl)pyridazin-4-yl]-4-methoxypiperidine-4-carboxylate (20.0 mg, 0.055 mmol) and tert-butyl 4-(methylamino)piperidine-1-carboxylate (14.9 mg, 0.069 mmol) were dissolved in 1.0 mL DMA. N,N-diisopropylethylamine (21.6 mg, 0.167 mmol) was added in one portion. [(dimethylamino)({[1,2,3]32olo[4,5-b]pyridin-3-yloxy})methylidene]dimethylazanium; hexafluoro-lambda5-phosphanuide (31.8 mg, 0.083 mmol) was added in one portion to the reaction mixture and the resulting solution was stirred and monitored by LCMS. Once complete, the reaction is diluted with water and extracted with EtOAc 3×5 mL. The organic phase is dried, filtered and concentrated in vacuo. The resulting residue is transferred was taken up in 1 mL DCM and 10% TFA in HFIP (1.0 mL) was added in one portion. The reaction was stirred for 1 h and then concentrated in vacuo to afford the desired product (31 mg, 129%) a brown solid. LCMS: C₂₃H₃₀FN₅O₃ requires: 443.2, found: m/z=444.2 [M+H]⁺

### Step 2: Synthesis of the Title Compound

1-[6-(5-fluoro-2-hydroxyphenyl)pyridazin-4-yl]-4-methoxy-N-methyl-N-(piperidin-4-yl)piperidine-4-carboxamide TFA (25.4 mg, 0.053 mmol) was dissolved in DMA (1.00 mL) and (3S)-3-(4-{3-oxo-1-oxa-8-azaspiro[4.5]decan-8-yl}phenyl)piperidine-2,6-dione (18.1 mg, 0.053 mmol) was added in one portion. N,N-diisopropylethylamine (34.2 mg, 0.265 mmol) was then added followed by sodium triacetoxyborohydride (33.7 mg, 0.159 mmol). The resulting reaction was stirred at RT overnight and monitored via LCMS. Once complete, the reaction was concentrated in vacuo. The residue was dissolved in DMF and purified via RP-HPLC to yield the title compound (10.0 mg, 21.6%) as a yellow solid. LCMS: C₄₂H₅₂FN₇O₆ requires: 769.4, found: m/z=770.5 [M+H]⁺

### Example 33

### RAC-N-CYCLOPROPYL-N-(1-((R)-8-(4-((S)-2,6-DIOXOPIPERIDIN-3-YL)PHENYL)-1-OXA-8-AZASPIRO[4.5]DECAN-3-YL)PIPERIDIN-4-YL)-1-(6-(2-HYDROXYPHENYL)PYRIDAZIN-4-YL)-METHOXYPIPERIDINE-4-CARBOXAMIDE

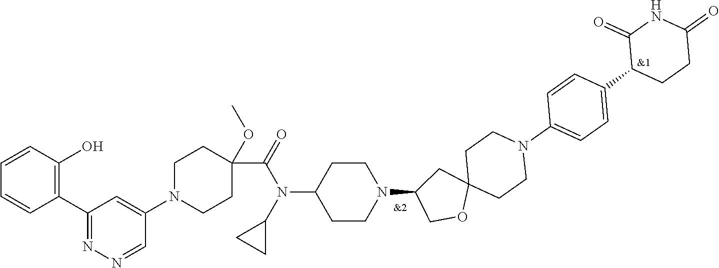 [SMILES: COC1(C(=O)N(C2CC2)C2CCN([C@@H]3COC4(CCN(c5ccc([C@H]6CCC(=O)NC6=O)cc5)CC4)C3)CC2)CCN(c2cnnc(-c3ccccc3O)c2)CC1]

### Step 1: Synthesis of N-cyclopropyl-1-(6-(2-hydroxyphenyl)pyridazin-4-yl)-4-methoxy-N-(piperidin-4-yl)piperidine-4-carboxamide

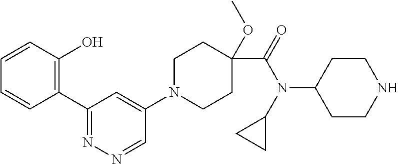 [SMILES: COC1(C(=O)N(C2CCNCC2)C2CC2)CCN(c2cnnc(-c3ccccc3O)c2)CC1]

To a solution of lithio 4-methoxy-1-{6-[2-(methoxymethoxy)phenyl]pyridazin-4-yl}piperidine-4-carboxylate (2.70 g, 7.1176 mmol) and tert-butyl 4-(cyclopropylamino)piperidine-1-carboxylate (2.57 g, 10.6764 mmol) in DCM (40.00 mL) was added N,N-diisopropylethylamine (4.97 mL, 3.68 g, 28.4705 mmol). Then bis(2-oxo-1,3-oxazolidin-3-yl)phosphinoyl chloride (5.44 g, 21.3529 mmol) was added and the reaction mixture was stirred at 40° C. Then, the reaction was quenched with water, extracted with DCM 3×50 mL, dried, filtered and concentrated under reduced pressure. The crude material was purified via chromatography (MeOH:DCM, gradient) to yield the desired product as an orange foam. The material was dissolved in 10 mL methanol and 4M HCl in dioxane (10 mL) was added in one portion. The reaction was stirred at RT for 1 hour and then concentrated in vacuo to yield the desired product (2.86 g, 86%) as a white solid. LCMS: C₂₅H₃₃N₅O₃ requires: 451.2, found: m/z=452.3 [M+H]⁺

### Step 2: Synthesis of the Title Compound

N-cyclopropyl-1-[6-(2-hydroxyphenyl)pyridazin-4-yl]-4-methoxy-N-(piperidin-4-yl)piperidine-4-carboxamide hydrochloride (23.00 mg, 0.0471 mmol) was dissolved in DMA (1.00 mL) and (3S)-3-(4-{3-oxo-1-oxa-8-azaspiro[4.5]decan-8-yl}phenyl)piperidine-2,6-dione (16.1 mg, 0.047 mmol) was added in one portion. N,N-diisopropylethylamine (30.5 mg, 0.235 mmol) was then added followed by sodium triacetoxyborohydride (29.9 mg, 0.141 mmol). The resulting reaction was stirred at RT overnight and monitored via LCMS. Once complete, the reaction was concentrated in vacuo. The residue was dissolved in DMF and purified via RP-HPLC to yield the title compound (12.6 mg, 33.3%) as a white solid. LCMS: C₄₄H₅₅N₇O₆ requires: 777.4, found: m/z=778.4 [M+H]⁺.

### Example 34

### (R)—N-(1-(2-(1-(4-(2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-4-METHOXY-N-PROPYLPIPERIDINE-4-CARBOXAMIDE

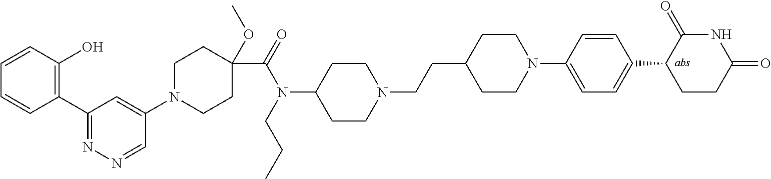 [SMILES: CCCN(C(=O)C1(OC)CCN(c2cnnc(-c3ccccc3O)c2)CC1)C1CCN(CCC2CCN(c3ccc([C@H]4CCC(=O)NC4=O)cc3)CC2)CC1]

### Step 1: Synthesis of 1-(6-(2-hydroxyphenyl)pyridazin-4-yl)-4-methoxy-N-(piperidin-4-yl)-N-propylpiperidine-4-carboxamide

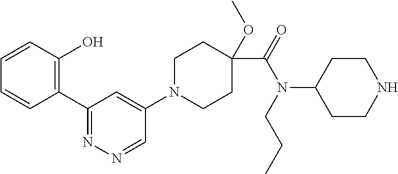 [SMILES: CCCN(C(=O)C1(OC)CCN(c2cnnc(-c3ccccc3O)c2)CC1)C1CCNCC1]

To a solution of lithio 4-methoxy-1-{6-[2-(methoxymethoxy)phenyl]pyridazin-4-yl}piperidine-4-carboxylate (500 mg, 1.31 mmol) and tert-butyl 4-(propylamino)piperidine-1-carboxylate (479 mg, 1.97 mmol) in DCM (10.00 mL) was added N,N-diisopropylethylamine (0.92 mL, 681 mg, 5.27 mmol). Then bis(2-oxo-1,3-oxazolidin-3-yl)phosphinoyl chloride (1006 mg, 3.95 mmol) was added and the reaction mixture was stirred at 40° C. Then, the reaction was quenched with water, extracted with DCM 3×50 mL, dried, filtered and concentrated under reduced pressure. The crude material was purified via chromatography (MeOH:DCM, gradient) to yield the desired product as an orange foam. The material was dissolved in 5 mL methanol and 4M HCl in dioxane (5 mL) was added in one portion. The reaction was stirred at RT for 1 hour and then concentrated in vacuo to yield the desired product (645 mg, 89%) as a white solid. LCMS: C₂₅H₃₅N₅O₃ requires: 453.2, found: m/z=454.2 [M+H]⁺

### Step 2: Synthesis of the Title Compound

1-[6-(2-hydroxyphenyl)pyridazin-4-yl]-4-methoxy-N-(piperidin-4-yl)-N-propylpiperidine-4-carboxamide hydrochloride (250 mg, 0.510 mmol) was dissolved in MeCN (2.50 mL) and 2-(1-{4-[(3R)-2,6-dioxopiperidin-3-yl]phenyl}piperidin-4-yl)acetaldehyde (176 mg, 0.561 mmol) was added in one portion. The flask was agitated and then MeOH (0.75 mL) was slowly added until all the solids have dissolved. Sodium triacetoxyborohydride (324 mg, 1.53 mmol) was then added in one portion and the reaction was stirred at RT for 30 mins. After 30 mins, the reaction was dissolved in 10 ml DCM. Sat. NaHCO₃ was then added and the reaction was extracted with DCM 3×10 mL. The organic phases were dried, concentrated in vacuo and then purified by SFC chiral separation to yield the title compound (256 mg, 66.7%) as a yellow solid. LCMS: C₄₃H₅₇N₇O₅ requires: 751.4, found: m/z=752.4 [M+H]⁺

### Example 35

### (R)—N-CYCLOPROPYL-N-(1-(2-(1-(4-(2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-4-METHOXYPIPERIDINE-4-CARBOXAMIDE

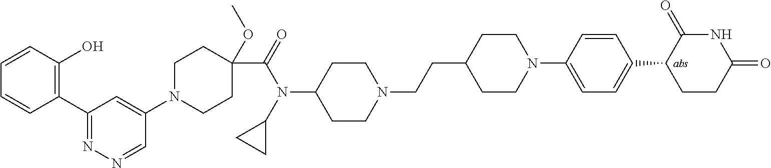 [SMILES: COC1(C(=O)N(C2CC2)C2CCN(CCC3CCN(c4ccc([C@H]5CCC(=O)NC5=O)cc4)CC3)CC2)CCN(c2cnnc(-c3ccccc3O)c2)CC1]

### Step 1: Synthesis of the Title Compound

N-cyclopropyl-1-[6-(2-hydroxyphenyl)pyridazin-4-yl]-4-methoxy-N-(piperidin-4-yl)piperidine-4-carboxamide hydrochloride (2.00 g, 4.09 mmol) was dissolved in MeCN (20.00 mL) and 2-(1-{4-[(3R)-2,6-dioxopiperidin-3-yl]phenyl}piperidin-4-yl)acetaldehyde (1.42 g, 4.51 mmol) was added in one portion. The flask was agitated and then MeOH (6.00 mL) was slowly added until all the solids have dissolved. Sodium triacetoxyborohydride (2.61 g, 12.3 mmol) was then added in one portion and the reaction was stirred at RT for 30 mins. After 30 mins, the reaction was dissolved in 50 ml DCM. water (30.0 mL) was then added and the layers were separated. The water layer was extracted with DCM 2×50 mL and the combined organic fractions were dried, concentrated in vacuo and then purified by SFC chiral separation to yield the title compound (1.67 g, 53.0%) as a white/yellow solid. LCMS: C₄₃H₅₅N₇O₅ requires: 749.4, found: m/z=750.3 [M+H]⁺

### Example 36

### (S)—N-(1-(2-(1-(4-(2,6-DIOXOPIPERIDIN-3-YL)PHENYL)PIPERIDIN-4-YL)ETHYL)PIPERIDIN-4-YL)-N-ETHYL-1-(6-(2-HYDROXYPHENYL)PYRIDAZIN-4-YL)-4-METHOXYPIPERIDINE-4-CARBOXAMIDE

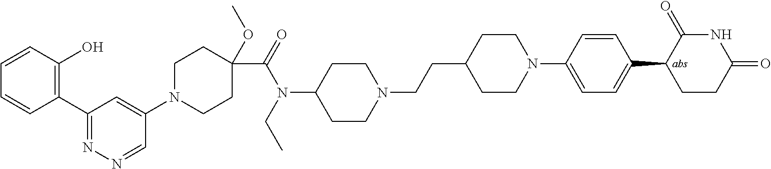 [SMILES: CCN(C(=O)C1(OC)CCN(c2cnnc(-c3ccccc3O)c2)CC1)C1CCN(CCC2CCN(c3ccc([C@@H]4CCC(=O)NC4=O)cc3)CC2)CC1]

### Step 1: Synthesis of the Title Compound

N-ethyl-1-[6-(2-hydroxyphenyl)pyridazin-4-yl]-4-methoxy-N-(piperidin-4-yl)piperidine-4-carboxamide hydrochloride (1.83 g, 3.84 mmol) was dissolved in MeCN (20.00 mL) and 2-(1-{4-[(3R)-2,6-dioxopiperidin-3-yl]phenyl}piperidin-4-yl)acetaldehyde (1.33 g, 4.22 mmol) was added in one portion. The flask was agitated and then MeOH (6.00 mL) was slowly added until all the solids have dissolved. Sodium triacetoxyborohydride (2.44 g, 11.5 mmol) was then added in one portion and the reaction was stirred at RT for 30 mins. After 30 mins, the reaction was dissolved in 50 ml DCM. Water (30.0 mL) was then added and the layers were separated. The water layer was extracted with DCM 2×50 mL and the combined organic fractions were dried, concentrated in vacuo and then purified by SFC chiral separation to yield the title compound (285 mg, 10.0%) as a white solid. LCMS: C₄₂H₅₅N₇O₅ requires: 737.4, found: m/z=738.4 [M+H]⁺.

### Example 37

### (R)—N-(1-(2-(1-(4-(2,6-DIOXOPIPERIDIN-3-YL)PHENYL)PIPERIDIN-4-YL)ETHYL)PIPERIDIN-4-YL)-1-(6-(3-FLUORO-2-HYDROXYPHENYL)PYRIDAZIN-4-YL)-4-METHOXYPIPERIDINE-4-CARBOXAMIDE

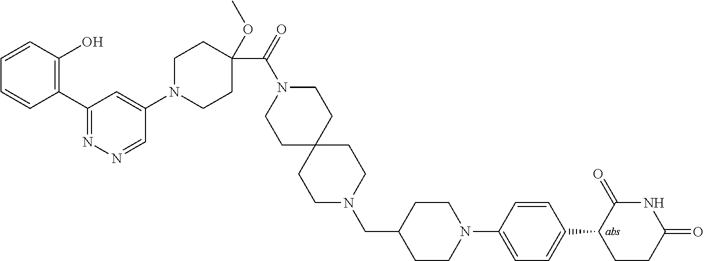 [SMILES: COC1(C(=O)N2CCC3(CCN(CC4CCN(c5ccc([C@H]6CCC(=O)NC6=O)cc5)CC4)CC3)CC2)CCN(c2cnnc(-c3ccccc3O)c2)CC1]

### Step 1: Synthesis of (1-(6-(2-hydroxyphenyl)pyridazin-4-yl)-4-methoxypiperidin-4-yl)(3,9-diazaspiro[5.5]undecan-3-yl)methanone

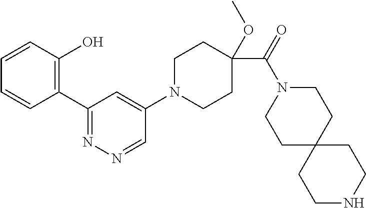 [SMILES: COC1(C(=O)N2CCC3(CCNCC3)CC2)CCN(c2cnnc(-c3ccccc3O)c2)CC1]

4-cyclopropoxy-1-[6-(2-hydroxyphenyl)pyridazin-4-yl]piperidine-4-carboxylic acid (30.0 mg, 0.084 mmol) and tert-butyl 3,9-diazaspiro[5.5]undecane-3-carboxylate (21.4 mg, 0.084 mmol) were dissolved in 1.0 mL DMA. N,N-diisopropylethylamine (0.04 mL, 32.7 mg, 0.253 mmol) was added in one portion. [(dimethylamino)({[1,2,3]triazolo[4,5-b]pyridin-3-yloxy})methylidene]dimethylazanium; hexafluoro-lambda5-phosphanuide (48.1 mg, 0.126 mmol) was added in one portion to the reaction mixture and the resulting solution was stirred and monitored by LCMS. Once reaction is complete, the reaction is diluted with water and extracted 3× with EtOAc. The organic phase is collected and concentrated in vacuo. The material was dissolved in 1 mL methanol and 4M HCl in dioxane (1.0 mL) was added in one portion. The reaction was stirred at RT for 1 hour and then concentrated in vacuo to yield the desired product (50.0 mg, 99%) as a white solid. LCMS: C₂₆H₃₅N₅O₃ requires: 465.2, found: m/z=466.2 [M+H]⁺

### Step 2: Synthesis of the Title Compound

2-[5-(4-{3,9-diazaspiro[5.5]undecane-3-carbonyl}-4-methoxypiperidin-1-yl)pyridazin-3-yl]phenol; trifluoroacetic acid (52.8 mg, 0.091 mmol) was dissolved in DMA (1.00 mL) and 1-{4-[(3R)-2,6-dioxopiperidin-3-yl]phenyl}piperidine-4-carbaldehyde (27.3 mg, 0.091 mmol) was added in one portion. N,N-diisopropylethylamine (0.08 mL, 58.8 mg, 0.455 mmol) was then added followed by sodium triacetoxyborohydride (57.9 mg, 0.273 mmol). The resulting reaction was stirred at RT for 1-2 h and monitored via LCMS. Once complete, the reaction was concentrated in vacuo. The residue was dissolved in DMF and purified via RP-HPLC to yield the title compound (11.4 mg, 56.6%) as a tan solid. LCMS: C₄₃H₅₅N₇O₅ requires: 749.4, found: m/z=750.4 [M+H]⁺.

### Example 38

### (R)—N-(1-(2-(1-(4-(2,6-DIOXOPIPERIDIN-3-YL)PHENYL)PIPERIDIN-4-YL)ETHYL)PIPERIDIN-4-YL)-1-(6-(3-FLUORO-2-HYDROXYPHENYL)PYRIDAZIN-4-YL)-4-METHOXYPIPERIDINE-4-CARBOXAMIDE

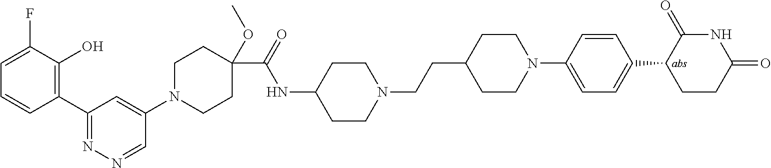 [SMILES: COC1(C(=O)NC2CCN(CCC3CCN(c4ccc([C@H]5CCC(=O)NC5=O)cc4)CC3)CC2)CCN(c2cnnc(-c3cccc(F)c3O)c2)CC1]

### Step 1: Synthesis of 1-(6-(3-fluoro-2-hydroxyphenyl)pyridazin-4-yl)-4-methoxy-N-(piperidin-4-yl)piperidine-4-carboxamide

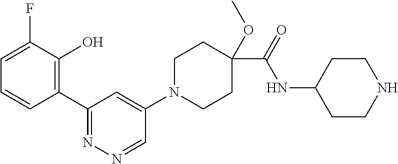 [SMILES: COC1(C(=O)NC2CCNCC2)CCN(c2cnnc(-c3cccc(F)c3O)c2)CC1]

dilithium(1+) 1-[6-(3-fluoro-2-oxidophenyl)pyridazin-4-yl]-4-methoxypiperidine-4-carboxylate (20.0 mg, 0.055 mmol) and tert-butyl 4-aminopiperidine-1-carboxylate (14.9 mg, 0.069 mmol) were dissolved in 1.0 mL DMA. N,N-diisopropylethylamine (21.6 mg, 0.167 mmol) was added in one portion. [(dimethylamino)({[1,2,3]triazolo[4,5-b]pyridin-3-yloxy})methylidene]dimethylazanium; hexafluoro-lambda5-phosphanuide (31.8 mg, 0.083 mmol) was added in one portion to the reaction mixture and the resulting solution was stirred and monitored by LCMS. Once complete, the reaction is diluted with water and extracted with EtOAc 3×5 mL. The organic phase is dried, filtered and concentrated in vacuo. The resulting residue is transferred was taken up in 1 mL DCM and 10% TFA in HFIP (1.0 mL) was added in one portion. The reaction was stirred for 1 h and then concentrated in vacuo to afford the desired product (32 mg, 130%) as a yellow solid. LCMS: C₂₂H₂₈FN₅O₃ requires: 429.2, found: m/z=430.2 [M+H]⁺

### Step 2: Synthesis of the Title Compound

1-[6-(3-fluoro-2-hydroxyphenyl)pyridazin-4-yl]-4-methoxy-N-(piperidin-4-yl)piperidine-4-carboxamide (11.9 mg, 0.026 mmol) was dissolved in DMA (1.00 mL) and 2-(1-{4-[(3R)-2,6-dioxopiperidin-3-yl]phenyl}piperidin-4-yl)acetaldehyde (9.81 mg, 0.031 mmol) was added in one portion. N,N-diisopropylethylamine (0.02 mL, 16.8 mg, 0.130 mmol) was then added. The solution was stirred for 30 mins before adding sodium triacetoxyborohydride (16.5 mg, 0.078 mmol). The resulting reaction was stirred at RT for 1-2 h and monitored via LCMS. Once complete, the reaction was concentrated in vacuo. The residue was dissolved in DMF and purified via RP-HPLC to yield the title compound (11.4 mg, 56.6%) as a tan solid. LCMS: C₄₁H₅₂FN₇O₅ requires: 727.3, found: m/z=727.4 [M+H]⁺

### Example 39

### (R)—N-(1-(2-(1-(4-(2,6-DIOXOPIPERIDIN-3-YL)PHENYL)PIPERIDIN-4-YL)ETHYL)PIPERIDIN-4-YL)-1-(6-(5-FLUORO-2-HYDROXYPHENYL)PYRIDAZIN-4-YL)-4-METHOXY-N-METHYLPIPERIDINE-4-CARBOXAMIDE

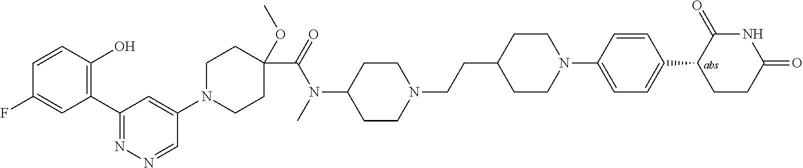 [SMILES: COC1(C(=O)N(C)C2CCN(CCC3CCN(c4ccc([C@H]5CCC(=O)NC5=O)cc4)CC3)CC2)CCN(c2cnnc(-c3cc(F)ccc3O)c2)CC1]

### Step 1: Synthesis of the Title Compound

1-[6-(5-fluoro-2-hydroxyphenyl)pyridazin-4-yl]-4-methoxy-N-methyl-N-(piperidin-4-yl)piperidine-4-carboxamide hydrochloride (25.4 mg, 0.053 mmol) was dissolved in DMA (1.00 mL) and 2-(1-{4-[(3R)-2,6-dioxopiperidin-3-yl]phenyl}piperidin-4-yl)acetaldehyde (19.9 mg, 0.063 mmol) was added in one portion. N,N-diisopropylethylamine (0.05 mL, 34.2 mg, 0.265 mmol) was then added followed by sodium triacetoxyborohydride (33.7 mg, 0.159 mmol). The resulting reaction was stirred at RT for 1-2 h and monitored via LCMS. Once complete, the reaction was concentrated in vacuo. The residue was dissolved in DMF and purified via RP-HPLC to yield the title compound (10.6 mg, 23.9%) as a white solid. LCMS: C₄₁H₅₂FN₇O₅ requires: 741.4, found: m/z=742.4 [M+H]⁺

### Example 40

### (S)—N-(1-(2-(1-(4-(2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-4-METHOXY-N-METHYLPIPERIDINE-4-CARBOXAMIDE

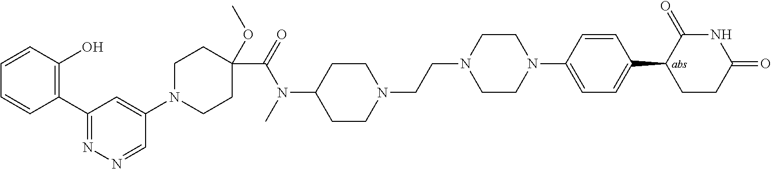 [SMILES: COC1(C(=O)N(C)C2CCN(CCN3CCN(c4ccc([C@@H]5CCC(=O)NC5=O)cc4)CC3)CC2)CCN(c2cnnc(-c3ccccc3O)c2)CC1]

### Step 1: Synthesis of the Title Compound

1-[6-(2-hydroxyphenyl)pyridazin-4-yl]-4-methoxy-N-methyl-N-(piperidin-4-yl)piperidine-4-carboxamide; trifluoroacetic acid (81.9 mg, 0.151 mmol) was dissolved in DMA (1.00 mL) and 2-(1-{4-[(3S)-2,6-dioxopiperidin-3-yl]phenyl}piperidin-4-yl)acetaldehyde (57.2 mg, 0.182 mmol) was added in one portion. N,N-diisopropylethylamine (0.13 mL, 98.1 mg, 0.759 mmol) was then added followed by sodium triacetoxyborohydride (96.5 mg, 0.455 mmol). The resulting reaction was stirred at RT for 1-2 hrs and monitored via LCMS. Once complete, the reaction was concentrated in vacuo. The residue was dissolved in DMF and purified via RP-HPLC to yield the title compound (28.9 mg, 23.9%) as a white solid. LCMS: C₄₁H₅₃N₇O₅ requires: 723.4, found: m/z=724.7 [M+H]⁺

### Example 41

### RAC-N-(1-((R)-8-(4-((S)-2,6-DIOXOPIPERIDIN-3-YL)PHENYL)-1-OXA-8-AZASPIRO[4.5]DECAN-3-YL)PIPERIDIN-4-YL)-1-(6-(2-HYDROXYPHENYL)PYRIDAZIN-4-YL)-4-METHOXYPIPERIDINE-4-CARBOXAMIDE

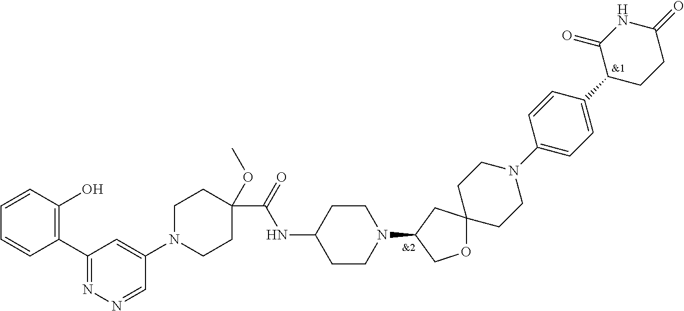 [SMILES: COC1(C(=O)NC2CCN([C@@H]3COC4(CCN(c5ccc([C@H]6CCC(=O)NC6=O)cc5)CC4)C3)CC2)CCN(c2cnnc(-c3ccccc3O)c2)CC1]

### Step 1: Synthesis of the Title Compound

1-[6-(2-hydroxyphenyl)pyridazin-4-yl]-4-methoxy-N-(piperidin-4-yl)piperidine-4-carboxamide; trifluoroacetic acid (13.0 mg, 0.023 mmol) was dissolved in DMA (1.00 mL) and rac-(3R)-3-(4-{3-oxo-1-oxa-8-azaspiro[4.5]decan-8-yl}phenyl)piperidine-2,6-dione (9.37 mg, 0.028 mmol) was added in one portion. N,N-diisopropylethylamine (0.02 mL, 15.1 mg, 0.117 mmol) was then added followed by sodium triacetoxyborohydride (14.9 mg, 0.071 mmol). The resulting reaction was stirred at RT for 1-2 hrs and monitored via LCMS. Once complete, the reaction was concentrated in vacuo. The residue was dissolved in DMF and purified via RP-HPLC to yield the title compound (2.9 mg, 15.5%) as a white solid. LCMS: C₄₁H₅₁N₇O₆ requires: 737.3, found: m/z=738.4 [M+H]⁺

### Example 42

### RAC-(R)—N-(1-(2-(4-(4-(2,6-DIOXOPIPERIDIN-3-YL)PHENYL)PIPERIDIN-1-YL)ETHYL)PIPERIDIN-4-YL)-1-(6-(2-HYDROXYPHENYL)PYRIDAZIN-4-YL)-4-METHOXY-N-METHYLPIPERIDINE-4-CARBOXAMIDE

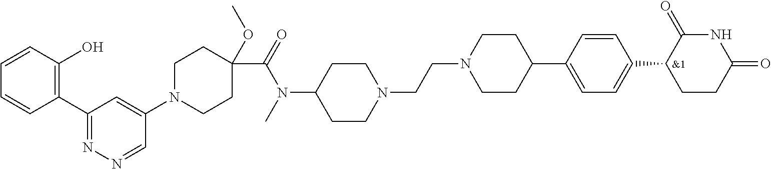 [SMILES: COC1(C(=O)N(C)C2CCN(CCN3CCC(c4ccc([C@H]5CCC(=O)NC5=O)cc4)CC3)CC2)CCN(c2cnnc(-c3ccccc3O)c2)CC1]

### Step 1: Synthesis of rac-(R)-3-(4-(1-(2-chloroethyl)piperidin-4-yl)phenyl)piperidine-2,6-dione

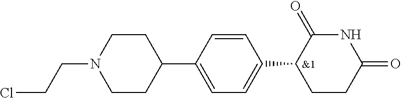 [SMILES: O=C1CC[C@H](c2ccc(C3CCN(CCCl)CC3)cc2)C(=O)N1]

To a solution of 3-[4-(piperidin-4-yl)phenyl]piperidine-2,6-dione hydrochloride (30.00 mg, 0.10 mmol) in DMF (1.00 mL) and N,N-diisopropylethylamine (50.90 μL, 0.04 g, 0.29 mmol), was added 1-bromo-2-chloroethane (81.00 μL, 139.32 mg, 0.97 mmol) and the reaction mixture was stirred at 45° C. Solvent was removed under reduced pressure and the crude product was used in the next step without further purification. LCMS: C₁₈H₂₃ClN₂O₂ requires 334.8, found: m/z=335.4 [M+H]⁺.

### Step 2: Synthesis of the Title Compound

To a vial containing 1-[6-(2-hydroxyphenyl)pyridazin-4-yl]-4-methoxy-N-methyl-N-(piperidin-4-yl)piperidine-4-carboxamide; trifluoroacetaldehyde (10.00 mg, 0.02 mmol) and rac-(3R)-3-{4-[l-(2-chloroethyl)piperidin-4-yl]phenyl}piperidine-2,6-dione (12.79 mg, 0.04 mmol) in DMA (0.50 mL) was added N,N-diisopropylethylamine (16.68 μL, 0.01 g, 0.10 mmol) followed by the addition of potassium iodide (3.20 mg, 0.02 mmol) and stirred at 70° C. until complete by LCMS. Once complete, the solution was injected onto RP—FC and purified with a gradient of 5-95% MeCN in H₂O to afford the title compound (1.40 mg, 9% yield) as an off-white solid. LCMS: C₄₁H₅₃N₇O₅ requires 723.4, found: m/z=724.3 [M+H]⁺.

### Example 43

### RAC-N-(1-((R)-8-(4-((S)-2,6-DIOXOPIPERIDIN-3-YL)PHENYL)-1-OXA-8-AZASPIRO[4.5]DECAN-3-YL)PIPERIDIN-4-YL)-1-(6-(2-HYDROXYPHENYL)PYRIDAZIN-4-YL)-4-METHOXY-N-METHYLPIPERIDINE-4-CARBOXAMIDE

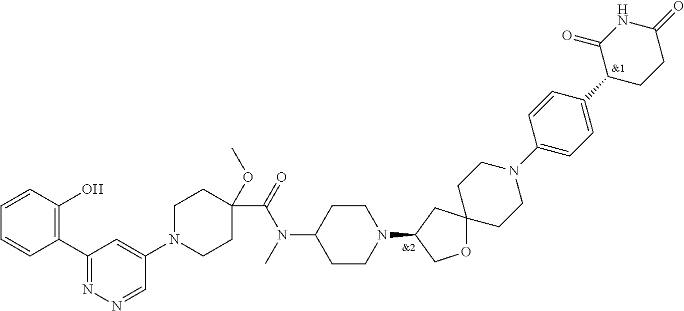 [SMILES: COC1(C(=O)N(C)C2CCN([C@@H]3COC4(CCN(c5ccc([C@H]6CCC(=O)NC6=O)cc5)CC4)C3)CC2)CCN(c2cnnc(-c3ccccc3O)c2)CC1]

### Step 1: Synthesis of the Title Compound

1-[6-(2-hydroxyphenyl)pyridazin-4-yl]-4-methoxy-N-methyl-N-(piperidin-4-yl)piperidine-4-carboxamide; trifluoroacetic acid (23.0 mg, 0.043 mmol) was dissolved in DMA (1.00 mL) and rac-(3R)-3-(4-{3-oxo-1-oxa-8-azaspiro[4.5]decan-8-yl}phenyl)piperidine-2,6-dione (17.5 mg, 0.051 mmol) was added in one portion. N,N-diisopropylethylamine (27.5 mg, 0.213 mmol) was then added followed by sodium triacetoxyborohydride (27.1 mg, 0.128 mmol). The resulting reaction was stirred at RT for 1-2 hrs and monitored via LCMS. Once complete, the reaction was concentrated in vacuo. The residue was dissolved in DMF and purified via RP-HPLC to yield the title compound (5.3 mg, 14.4%) as a tan solid. LCMS: C₄₂H₅₃N₇O₆ requires: 751.4, found: m/z=752.5 [M+H]⁺

### Example 44

### RAC-N-(1-((R)-8-(4-((S)-2,6-DIOXOPIPERIDIN-3-YL)PHENYL)-1-OXA-8-AZASPIRO[4.5]DECAN-3-YL)PIPERIDIN-4-YL)-1-(6-(2-HYDROXYPHENYL)PYRIDAZIN-4-YL)-4-CYCLOBUTOXY-N-METHYLPIPERIDINE-4-CARBOXAMIDE

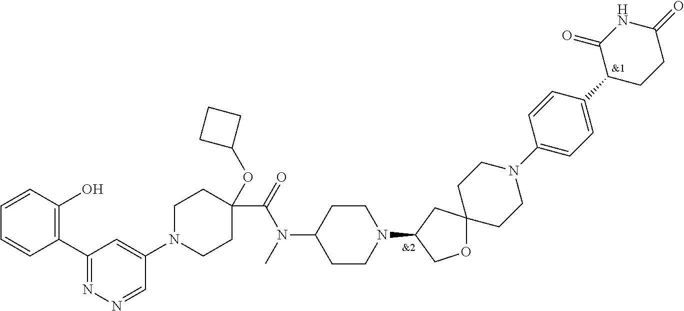 [SMILES: CN(C(=O)C1(OC2CCC2)CCN(c2cnnc(-c3ccccc3O)c2)CC1)C1CCN([C@@H]2COC3(CCN(c4ccc([C@H]5CCC(=O)NC5=O)cc4)CC3)C2)CC1]

The title compound was prepared according to Example 43 using 1-[6-(2-hydroxyphenyl)pyridazin-4-yl]-4-cyclobutoxy-N-methyl-N-(piperidin-4-yl)piperidine-4-carboxamide.

### Example 45

### RAC-N-(1-((R)-8-(4-((S)-2,6-DIOXOPIPERIDIN-3-YL)PHENYL)-1-OXA-8-AZASPIRO[4.5]DECAN-3-YL)PIPERIDIN-4-YL)-4-ETHOXY-1-(6-(2-HYDROXYPHENYL)PYRIDAZIN-4-YL)-N-METHYLPIPERIDINE-4-CARBOXAMIDE

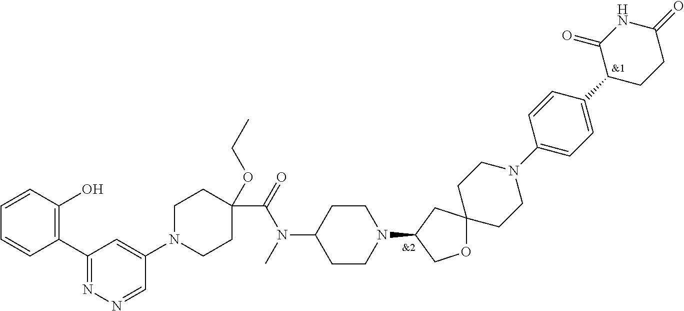 [SMILES: CCOC1(C(=O)N(C)C2CCN([C@@H]3COC4(CCN(c5ccc([C@H]6CCC(=O)NC6=O)cc5)CC4)C3)CC2)CCN(c2cnnc(-c3ccccc3O)c2)CC1]

### Step 1: Synthesis of the Title Compound

4-ethoxy-1-[6-(2-hydroxyphenyl)pyridazin-4-yl]-N-methyl-N-(piperidin-4-yl)piperidine-4-carboxamide; trifluoroacetic acid (13.0 mg, 0.023 mmol) was dissolved in DMA (1.00 mL) and rac-(3R)-3-(4-{3-oxo-1-oxa-8-azaspiro[4.5]decan-8-yl}phenyl)piperidine-2,6-dione (9.37 mg, 0.028 mmol) was added in one portion. N,N-diisopropylethylamine (0.02 mL, 15.2 mg, 0.117 mmol) was then added followed by sodium triacetoxyborohydride (14.9 mg, 0.071 mmol). The resulting reaction was stirred at RT for 1-2 h and monitored via LCMS. Once complete, the reaction was concentrated in vacuo. The residue was dissolved in DMF and purified via RP-HPLC to yield the title compound (2.2 mg, 10.4%) as a white solid. LCMS: C₄₃H₅₅N₇O₆ requires: 765.4, found: m/z=766.4 [M+H]⁺

### Example 46

### RAC-(R)—N-(1-(2-(4-(4-(2,6-DIOXOPIPERIDIN-3-YL)PHENYL)PIPERAZIN-1-YL)ETHYL)PIPERIDIN-4-YL)-1-(6-(2-HYDROXYPHENYL)PYRIDAZIN-4-YL)-4-METHOXY-N-METHYLPIPERIDINE-4-CARBOXAMIDE

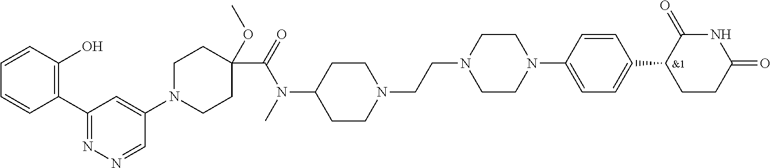 [SMILES: COC1(C(=O)N(C)C2CCN(CCN3CCN(c4ccc([C@H]5CCC(=O)NC5=O)cc4)CC3)CC2)CCN(c2cnnc(-c3ccccc3O)c2)CC1]

### Step 1: Synthesis of rac-(R)-3-(4-(4-(2-chloroethyl)piperazin-1-yl)phenyl)piperidine-2,6-dione

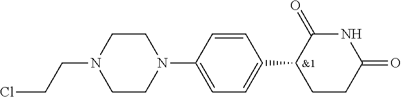 [SMILES: O=C1CC[C@H](c2ccc(N3CCN(CCCl)CC3)cc2)C(=O)N1]

To a solution of 3-[4-(piperazin-1-yl)phenyl]piperidine-2,6-dione; trifluoroacetic acid (50.00 mg, 0.13 mmol) in DMF (1.00 mL) and N,N-diisopropylethylamine (67.63 μL, 0.05 g, 0.39 mmol), was added 1-bromo-2-chloroethane (107.62 μL, 185.11 mg, 1.29 mmol) and the reaction mixture was stirred at 45° C. Solvent was removed under reduced pressure and the crude product was used in the next step without further purification. LCMS: C₁₈H₂₃ClN₂O₂ requires 335.1, found: m/z=336.2 [M+H]⁺.

### Step 2: Synthesis of the Title Compound

The title compound was prepared as described for Example 42, Step 2, using rac-(R)-3-(4-(4-(2-chloroethyl)piperazin-1-yl)phenyl)piperidine-2,6-dione and 1-(6-(2-hydroxyphenyl)pyridazin-4-yl)-4-methoxy-N-methyl-N-(piperidin-4-yl)piperidine-4-carboxamide to afford rac-(R)—N-(1-(2-(4-(4-(2,6-dioxopiperidin-3-yl)phenyl)piperazin-1-yl)ethyl)piperidin-4-yl)-1-(6-(2-hydroxyphenyl)pyridazin-4-yl)-4-methoxy-N-methylpiperidine-4-carboxamide (1.00 mg, 5% yield) as a white solid. LCMS: C₄₀H₅₂N₈O₅ requires 724.4, found: m/z=725.4 [M+H]⁺.

### Example 47

### (R)—N-(1-((1-(4-(2,6-DIOXOPIPERIDIN-3-YL)PHENYL)PIPERIDIN-4-YL)METHYL)PIPERIDIN-4-YL)-1-(6-(2-HYDROXYPHENYL)PYRIDAZIN-4-YL)-4-METHOXYPIPERIDINE-4-CARBOXAMIDE

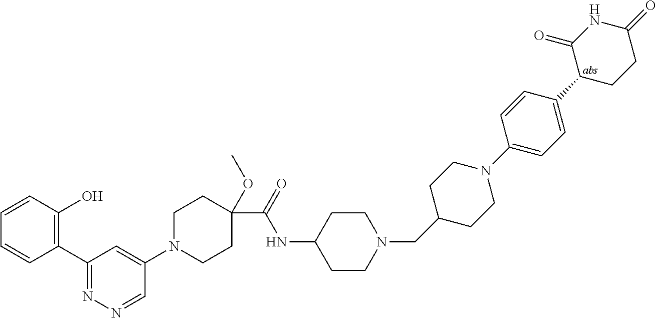 [SMILES: COC1(C(=O)NC2CCN(CC3CCN(c4ccc([C@H]5CCC(=O)NC5=O)cc4)CC3)CC2)CCN(c2cnnc(-c3ccccc3O)c2)CC1]

### Step 1: Synthesis of the Title Compound

1-[6-(2-hydroxyphenyl)pyridazin-4-yl]-4-methoxy-N-(piperidin-4-yl)piperidine-4-carboxamide; trifluoroacetic acid (13.0 mg, 0.023 mmol) was dissolved in DMA (1.00 mL) and 1-{4-[(3R)-2,6-dioxopiperidin-3-yl]phenyl}piperidine-4-carbaldehyde (9.37 mg, 0.028 mmol) was added in one portion. N,N-diisopropylethylamine (0.02 mL, 15.2 mg, 0.117 mmol) was then added followed by sodium triacetoxyborohydride (14.9 mg, 0.071 mmol). The resulting reaction was stirred at RT for 1-2 hrs and monitored via LCMS. Once complete, the reaction was concentrated in vacuo. The residue was dissolved in DMF and purified via RP-HPLC to yield the title compound (7.0 mg, 39.5%) as a white solid. LCMS: C₃₉H₄₉N₇O₅ requires: 695.3, found: m/z=696.4 [M+H]⁺

### Example 48

### RAC-N-(1-((R)-8-(4-((S)-2,6-DIOXOPIPERIDIN-3-YL)PHENYL)-1-OXA-8-AZASPIRO[4.5]DECAN-3-YL)PIPERIDIN-4-YL)-4-ETHOXY-1-(6-(2-HYDROXYPHENYL)PYRIDAZIN-4-YL)-N-METHYLPIPERIDINE-4-CARBOXAMIDE

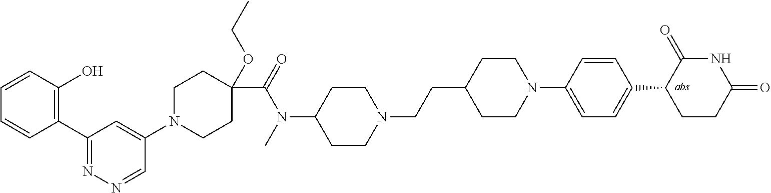 [SMILES: CCOC1(C(=O)N(C)C2CCN(CCC3CCN(c4ccc([C@H]5CCC(=O)NC5=O)cc4)CC3)CC2)CCN(c2cnnc(-c3ccccc3O)c2)CC1]

### Step 1: Synthesis of the Title Compound

4-ethoxy-1-[6-(2-hydroxyphenyl)pyridazin-4-yl]-N-methyl-N-(piperidin-4-yl)piperidine-4-carboxamide; trifluoroacetic acid (13.3 mg, 0.028 mmol) was dissolved in DMA (1.00 mL) and 2-(1-{4-[(3R)-2,6-dioxopiperidin-3-yl]phenyl}piperidin-4-yl)acetaldehyde (10.6 mg, 0.033 mmol) was added in one portion. N,N-diisopropylethylamine (0.02 mL, 18.1 mg, 0.140 mmol) was then added followed by sodium triacetoxyborohydride (17.8 mg, 0.084 mmol). The resulting reaction was stirred at RT for 1-2 h and monitored via LCMS. Once complete, the reaction was concentrated in vacuo. The residue was dissolved in DMF and purified via RP-HPLC to yield the title compound (16.3 mg, 71.0%) as a white solid. LCMS: C₄₂H₅₅N₇O₅ requires: 737.4, found: m/z=738.4 [M+H]⁺

### Example 49

### (R)—N-(1-(2-(1-(4-(2,6-DIOXOPIPERIDIN-3-YL)PHENYL)PIPERIDIN-4-YL)ETHYL)PIPERIDIN-4-YL)-N-ETHYL-1-(6-(2-HYDROXYPHENYL)PYRIDAZIN-4-YL)-4-METHOXYPIPERIDINE-4-CARBOXAMIDE

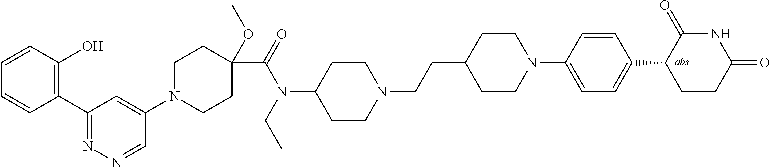 [SMILES: CCN(C(=O)C1(OC)CCN(c2cnnc(-c3ccccc3O)c2)CC1)C1CCN(CCC2CCN(c3ccc([C@H]4CCC(=O)NC4=O)cc3)CC2)CC1]

### Step 1: Synthesis of the Title Compound

N-ethyl-1-[6-(2-hydroxyphenyl)pyridazin-4-yl]-4-methoxy-N-(piperidin-4-yl)piperidine-4-carboxamide hydrochloride (1.83 g, 3.84 mmol) was dissolved in MeCN (20.0 mL) and 2-(1-{4-[(3R)-2,6-dioxopiperidin-3-yl]phenyl}piperidin-4-yl)acetaldehyde (1.33 g, 4.22 mmol) was added in one portion. The flask was agitated and then MeOH (6.00 mL) was slowly added until all the solids have dissolved. Sodium triacetoxyborohydride (2.44 g, 11.5 mmol) was then added in one portion and the reaction was stirred at RT for 30 mins. After 30 mins, the reaction was dissolved in 50 ml DCM. Water (30.0 mL) was then added and the layers were separated. The water layer was extracted with DCM 2×50 mL and the combined organic fractions were dried, concentrated in vacuo and then purified by SFC chiral separation to yield the title compound (2.41 g, 84.6%) as a tan solid. LCMS: C₄₂H₅₅N₇O₅ requires: 737.4, found: m/z=738.4 [M+H]⁺

### Example 50

### (R)-3-(4-(4-(2-(4-(1-(6-(2-HYDROXYPHENYL)PYRIDAZIN-4-YL)-4-METHOXYPIPERIDINE-4-CARBONYL)PIPERAZIN-1-YL)ETHYL)PIPERIDIN-1-YL)PHENYL)PIPERIDINE-2,6-DIONE

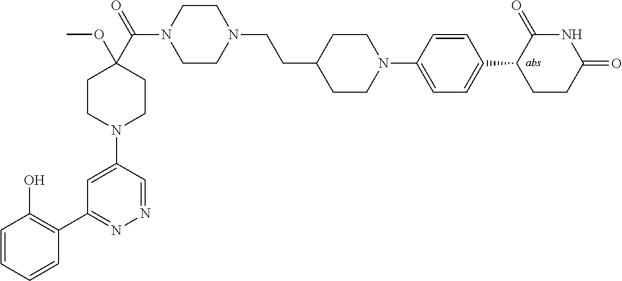 [SMILES: COC1(C(=O)N2CCN(CCC3CCN(c4ccc([C@H]5CCC(=O)NC5=O)cc4)CC3)CC2)CCN(c2cnnc(-c3ccccc3O)c2)CC1]

### Step 1: Synthesis of the Title Compound

2-{5-[4-methoxy-4-(piperazine-1-carbonyl)piperidin-1-yl]pyridazin-3-yl}phenol; trifluoroacetaldehyde (22.3 mg, 0.045 mmol) was dissolved in DMA (0.50 mL) and 2-(1-{4-[(3R)-2,6-dioxopiperidin-3-yl]phenyl}piperidin-4-yl)acetaldehyde (18.4 mg, 0.058 mmol) was added in one portion. N,N-diisopropylethylamine (0.04 mL, 29.1 mg, 0.225 mmol) was then added followed by sodium triacetoxyborohydride (28.6 mg, 0.135 mmol). The reaction was stirred at RT for 1-2 hrs and monitored via LCMS. Once complete, the reaction was concentrated in vacuo. The residue was dissolved in DMF and purified via RP-HPLC to yield the title compound (8.9 mg, 25.1%) as a white solid. LCMS: C₃₉H₄₉N₇O₅ requires: 695.3, found: m/z=696.4 [M+H]⁺

### Example 51

### (R)—N-(1-(2-(1-(3-(2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-4-METHOXY-N-METHYLPIPERIDINE-4-CARBOXAMIDE

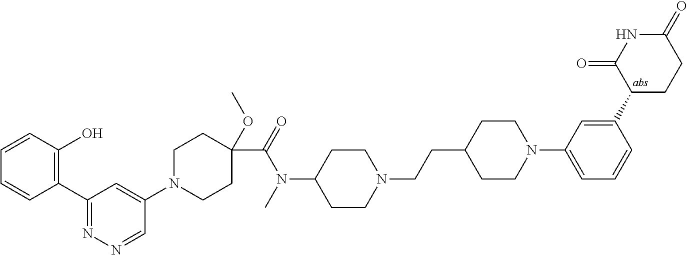 [SMILES: COC1(C(=O)N(C)C2CCN(CCC3CCN(c4cccc([C@H]5CCC(=O)NC5=O)c4)CC3)CC2)CCN(c2cnnc(-c3ccccc3O)c2)CC1]

### Step 1: Synthesis of the Title Compound

1-[6-(2-hydroxyphenyl)pyridazin-4-yl]-4-methoxy-N-methyl-N-(piperidin-4-yl)piperidine-4-carboxamide hydrochloride (15.0 mg, 0.032 mmol) and rac-2-(1-{3-[(3R)-2,6-dioxopiperidin-3-yl]phenyl}piperidin-4-yl)acetaldehyde (10.8 mg, 0.034 mmol) were dissolved in DMA (0.50 mL) and N,N-diisopropylethylamine (0.03 mL, 20.9 mg, 0.162 mmol) was added in one portion. Sodium triacetoxyborohydride (20.6 mg, 0.097 mmol) was added to the reaction mixture and the solution was stirred at RT overnight. Once complete, the reaction was concentrated in vacuo. The residue was dissolved in DMF and purified via RP-HPLC to yield the title compound (3.9 mg, 14.1%) as a yellow solid. LCMS: C₄₁H₅₃N₇O₅ requires: 723.4, found: m/z=724.4 [M+H]⁺

### Example 52

### (R)—N-(1-(2-(1-(3-(2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-4-METHOXY-N-METHYLPIPERIDINE-4-CARBOXAMIDE

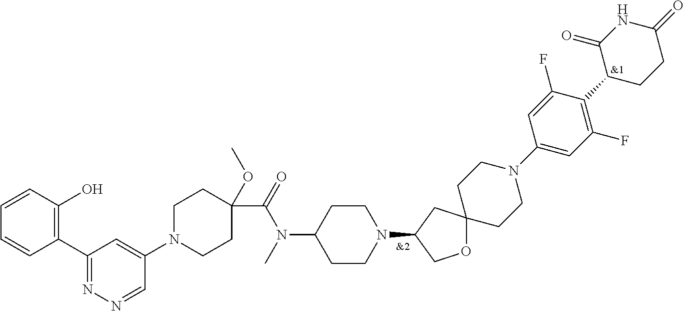 [SMILES: COC1(C(=O)N(C)C2CCN([C@@H]3COC4(CCN(c5cc(F)c([C@H]6CCC(=O)NC6=O)c(F)c5)CC4)C3)CC2)CCN(c2cnnc(-c3ccccc3O)c2)CC1]

### Step 1: Synthesis of the Title Compound

1-[6-(2-hydroxyphenyl)pyridazin-4-yl]-4-methoxy-N-methyl-N-(piperidin-4-yl)piperidine-4-carboxamide hydrochloride (15.0 mg, 0.032 mmol) and rac-(3R)-3-(2,6-difluoro-4-{3-oxo-1-oxa-8-azaspiro[4.5]decan-8-yl}phenyl)piperidine-2,6-dione (12.4 mg, 0.034 mmol) were dissolved in DMA (0.50 mL) and N,N-diisopropylethylamine (0.03 mL, 20.9 mg, 0.162 mmol) was added in one portion. sodium triacetoxyborohydride (20.6 mg, 0.097 mmol) was added to the reaction mixture and the solution was stirred at RT overnight. Once complete, the reaction was concentrated in vacuo. The residue was dissolved in DMF and purified via RP-HPLC to yield the title compound (3.1 mg, 11.3%) as a white solid. LCMS: C₄₂H₅₁F₂N₇O₆ requires: 787.3, found: m/z=788.4 [M+H]⁺

### Example 53

### RAC-(R)—N-(1-(2-(1-(5-(2,6-DIOXOPIPERIDIN-3-YL)PYRIDIN-2-YL)PIPERIDIN-4-YL)ETHYL)PIPERIDIN-4-YL)-1-(6-(2-HYDROXYPHENYL)PYRIDAZIN-4-YL)-4-METHOXY-N-METHYLPIPERIDINE-4-CARBOXAMIDE

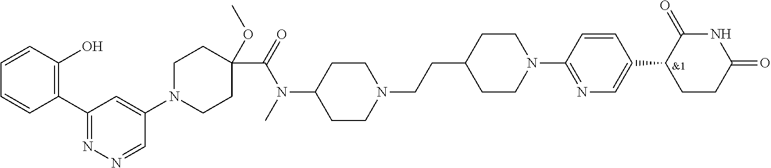 [SMILES: COC1(C(=O)N(C)C2CCN(CCC3CCN(c4ccc([C@H]5CCC(=O)NC5=O)cn4)CC3)CC2)CCN(c2cnnc(-c3ccccc3O)c2)CC1]

### Step 1: Synthesis of the Title Compound

The title compound was prepared as described for Example 1, Step 2, using 1-(6-(2-hydroxyphenyl)pyridazin-4-yl)-4-methoxy-N-methyl-N-(piperidin-4-yl)piperidine-4-carboxamide and rac-(R)-2-(1-(5-(2,6-dioxopiperidin-3-yl)pyridin-2-yl)piperidin-4-yl)acetaldehyde to afford rac-(R)—N-(1-(2-(1-(5-(2,6-dioxopiperidin-3-yl)pyridin-2-yl)piperidin-4-yl)ethyl)piperidin-4-yl)-N-ethyl-1-(6-(2-hydroxyphenyl)pyridazin-4-yl)-4-methoxypiperidine-4-carboxamide (8.50 mg, 33% yield) as a white solid. LCMS: C₄₀H₅₂N₈O₅ requires 724.4, found: m/z=725.2 [M+H]⁺.

### Example 54

### (RS)—N-(1-(2-(1-(4-((R)-2,6-DIOXOPIPERIDIN-3-YL)PHENYL)PIPERIDIN-4-YL)ETHYL)PIPERIDIN-4-YL)-N-ETHYL-1-(6-(2-HYDROXYPHENYL)PYRIDAZIN-4-YL)-3-METHOXYPYRROLIDINE-3-CARBOXAMIDE

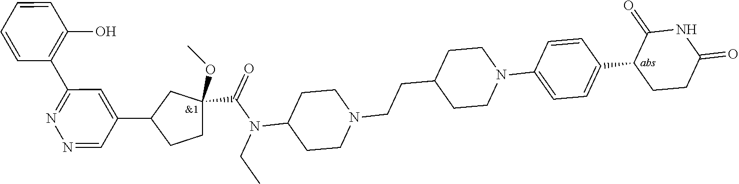 [SMILES: CCN(C(=O)[C@]1(OC)CCC(c2cnnc(-c3ccccc3O)c2)C1)C1CCN(CCC2CCN(c3ccc([C@H]4CCC(=O)NC4=O)cc3)CC2)CC1]

### Step 1: Synthesis of rac-(R)—N-ethyl-1-(6-(2-hydroxyphenyl)pyridazin-4-yl)-3-methoxy-N-(piperidin-4-yl)pyrrolidine-3-carboxamide hydrochloride

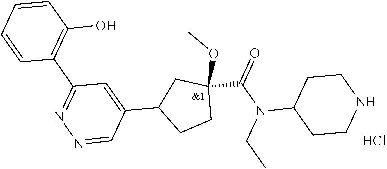 [SMILES: CCN(C(=O)[C@]1(OC)CCC(c2cnnc(-c3ccccc3O)c2)C1)C1CCNCC1 ; Cl]

This compound was prepare as escribe or e syn thesis of Example Step 1, using rac-(R)-3-methoxy-1-(6-(2-(methoxymethoxy)phenyl)pyridazin-4-yl)pyrrolidine-3-carboxylic acid (69.3 mg, 0.20 mmol) and tert-butyl 4-(ethylamino)piperidine-1-carboxylate to afford the desired compound as a yellow oil. LCMS C₂₃H₃₁N₅O₃ requires 425.2, found m/z=426.2 [M+H]⁺.

### Step 3: Synthesis of the Title Compound

The title compound was prepared as described for the synthesis of Example 2 Step 2, using rac-(R)—N-ethyl-1-(6-(2-hydroxyphenyl)pyridazin-4-yl)-3-methoxy-N-(piperidin-4-yl)pyrrolidine-3-carboxamide hydrochloride (58.0 mg, 0.14 mmol) to afford the title compound (32.5 mg, 29% yield) as a white solid. LCMS C₄₁H₅₃N₇O₅ requires 723.4, found m/z=724.4 [M+H]⁺.

### Example 55

### (RS)—N-(1-(2-(1-(4-((R)-2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-3-METHOXY-N-METHYLPYRROLIDINE-3-CARBOXAMIDE

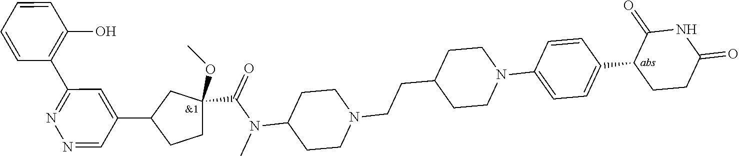 [SMILES: CO[C@@]1(C(=O)N(C)C2CCN(CCC3CCN(c4ccc([C@H]5CCC(=O)NC5=O)cc4)CC3)CC2)CCC(c2cnnc(-c3ccccc3O)c2)C1]

### Step 1: Synthesis of rac-(R)-1-(6-(2-hydroxyphenyl)pyridazin-4-yl)-3-methoxy-N-methyl-N-(piperidin-4-yl)pyrrolidine-3-carboxamide hydrochloride

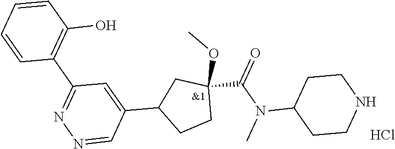 [SMILES: CO[C@@]1(C(=O)N(C)C2CCNCC2)CCC(c2cnnc(-c3ccccc3O)c2)C1 ; Cl]

This compound was prepared as described for the synthesis of Example 1 Step 1, using rac-(R)-3-methoxy-1-(6-(2-(methoxymethoxy)phenyl)pyridazin-4-yl)pyrrolidine-3-carboxylic acid (70.2 mgs, 0.20 mmol) and tert-butyl 4-(methylamino)piperidine-1-carboxylate to afford the desired compound as a yellow solid. LCMS C₂₂H₂₉N₅O₃ requires 411.2, found m/z=412.2 [M+H]⁺.

### Step 2: Synthesis of Title Compound

The title compound was prepared as described for the synthesis of Example 2 Step 2, using rac-(R)-1-(6-(2-hydroxyphenyl)pyridazin-4-yl)-3-methoxy-N-methyl-N-(piperidin-4-yl)pyrrolidine-3-carboxamide hydrochloride (14.8 mg, 0.04 mmol) to afford the title compound (15.3 mg, 51% yield) as a white solid. LCMS C₄₀H₅₁N₇O₅ requires 709.4, found m/z=710.4 [M+H]⁺.

### Example 56

### (R)—N-(1-(2-(1-(3-(2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-4-METHOXY-N-METHYLPIPERIDINE-4-CARBOXAMIDE

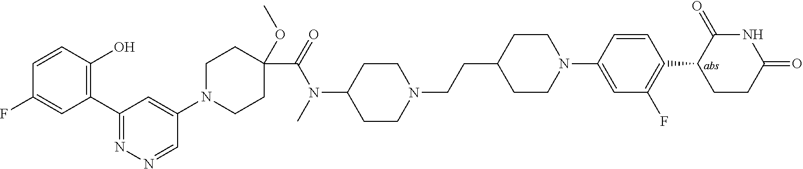 [SMILES: COC1(C(=O)N(C)C2CCN(CCC3CCN(c4ccc([C@H]5CCC(=O)NC5=O)c(F)c4)CC3)CC2)CCN(c2cnnc(-c3cc(F)ccc3O)c2)CC1]

### Step 1: Synthesis of the Title Compound

1-[6-(5-fluoro-2-hydroxyphenyl)pyridazin-4-yl]-4-methoxy-N-methyl-N-(piperidin-4-yl)piperidine-4-carboxamide hydrochloride (25.4 mg, 0.053 mmol) was dissolved in DMA (1.00 mL) and 2-(1-{4-[(3R)-2,6-dioxopiperidin-3-yl]-3-fluorophenyl}piperidin-4-yl)acetaldehyde (19.9 mg, 0.063 mmol) was added in one portion. N,N-diisopropylethylamine (34.25 mg, 0.2650 mmol) was then added followed by sodium triacetoxyborohydride (33.70 mg, 0.1590 mmol). The resulting reaction was stirred at RT for 1-2 hrs and monitored via LCMS. Once complete, the reaction was concentrated in vacuo. The residue was dissolved in DMF and purified via RP-HPLC to yield the title compound (14.0 mg, 31.9%). LCMS: C₄₁H₅₁F₂N₇O₅ requires: 759.4, found: m/z=760.5 [M+H]⁺

### Example 57

### (R)—N-(1-(2-(1-(4-(2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-4-(METHOXYMETHYL)-N-METHYLPIPERIDINE-4-CARBOXAMIDE

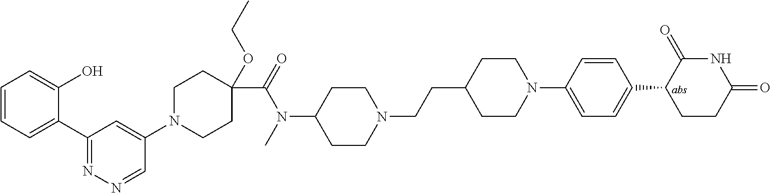 [SMILES: CCOC1(C(=O)N(C)C2CCN(CCC3CCN(c4ccc([C@H]5CCC(=O)NC5=O)cc4)CC3)CC2)CCN(c2cnnc(-c3ccccc3O)c2)CC1]

### Step 1: Synthesis of 1-(6-(2-hydroxyphenyl)pyridazin-4-yl)-4-(methoxymethyl)-N-methyl-N-(piperidin-4-yl)piperidine-4-carboxamide hydrochloride

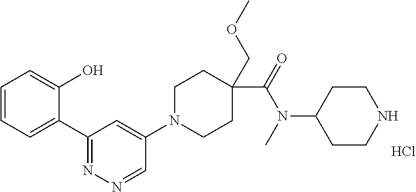 [SMILES: COCC1(C(=O)N(C)C2CCNCC2)CCN(c2cnnc(-c3ccccc3O)c2)CC1 ; Cl]

Synthesis of this compound was performed as described for Example 1 Step 1 using 1-(6-(2-(methoxymethoxy)phenyl)pyridazin-4-yl)-4-(methoxymethyl)piperidine-4-carboxylic acid (107 mg, 0.27 mmol) and tert-butyl 4-(methylamino)piperidine-1-carboxylate. LCMS C₂₄H₃₃N₅O₃ requires 439.3, found m/z 5=440.3 [M+H]⁺.

### Step 2: Synthesis of Title Compound

The title compound was prepared as described for Example 2 Step 2, using 1-(6-(2-hydroxyphenyl)pyridazin-4-yl)-4-(methoxymethyl)-N-methyl-N-(piperidin-4-yl)piperidine-4-carboxamide hydrochloride (21.2 mg, 0.048 mmol) to afford title compound (24.2 mg, 62% yield) as a white solid. LCMS C₄₂H₅₅N₇O₅ requires 737.4, found m/z=738.4 [M+H]⁺.

### Example 58

### (R)—N-(1-(2-(1-(4-(2,6-DIOXOPIPERIDIN-3-YL)PHENYL)PIPERIDIN-4-YL)ETHYL)PIPERIDIN-4-YL)-N-ETHYL-1-(6-(2-HYDROXYPHENYL)PYRIDAZIN-4-YL)-4-(METHOXYMETHYL)PIPERIDINE-4-CARBOXAMIDE

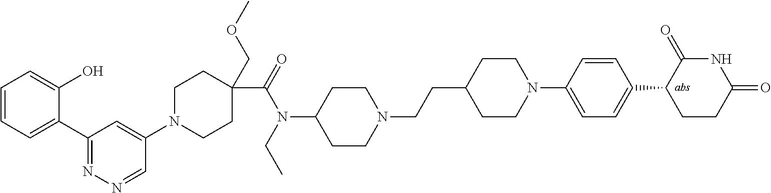 [SMILES: CCN(C(=O)C1(COC)CCN(c2cnnc(-c3ccccc3O)c2)CC1)C1CCN(CCC2CCN(c3ccc([C@H]4CCC(=O)NC4=O)cc3)CC2)CC1]

### Step 1: Synthesis of N-ethyl-1-(6-(2-hydroxyphenyl)pyridazin-4-yl)-4-(methoxymethyl)-N-(piperidin-4-yl)piperidine-4-carboxamide hydrochloride

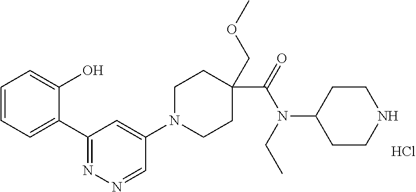 [SMILES: CCN(C(=O)C1(COC)CCN(c2cnnc(-c3ccccc3O)c2)CC1)C1CCNCC1 ; Cl]

The synthesis of this compound was performed as described for Example 2 Step 1, using 1-(6-(2-(methoxymethoxy)phenyl)pyridazin-4-yl)-4-(methoxymethyl)piperidine-4-carboxylic acid (111 mg, 0.32 mmol) and tert-butyl 4-(ethylamino)piperidine-1-carboxylate. LCMS C₂₅H₃₅N₅O₃ requires 453.3, found m/z=454.3 [M+H]⁺.

### Step 2: Synthesis of Title Compound

The title compound was prepared as described for Example 2 Step 2, using N-ethyl-1-(6-(2-hydroxyphenyl)pyridazin-4-yl)-4-(methoxymethyl)-N-(piperidin-4-yl)piperidine-4-carboxamide hydrochloride (42.9 mg, 0.10 mmol) to afford the title compound (5.1 mg, 6% yield) as a white solid. LCMS C₄₃H₅₇N₇O₅ requires 751.4, found m/z=752.4 [M+H]⁺.

### Example 59

### (R)—N-CYCLOPROPYL-N-(1-(2-(1-(4-(2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-4-(METHOXYMETHYL)PIPERIDINE-4-CARBOXAMIDE

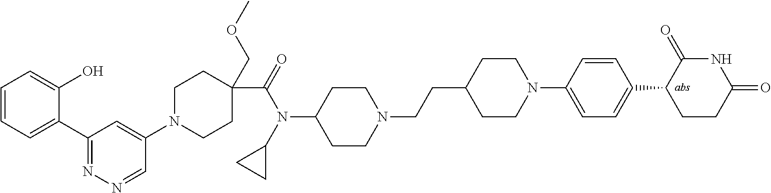 [SMILES: COCC1(C(=O)N(C2CC2)C2CCN(CCC3CCN(c4ccc([C@H]5CCC(=O)NC5=O)cc4)CC3)CC2)CCN(c2cnnc(-c3ccccc3O)c2)CC1]

### Step 1: Synthesis of N-cyclopropyl-1-(6-(2-hydroxyphenyl)pyridazin-4-yl)-4-(methoxymethyl)-N-(piperidin-4-yl)piperidine-4-carboxamide hydrochloride

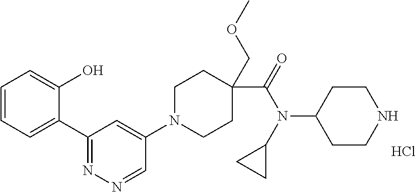 [SMILES: COCC1(C(=O)N(C2CCNCC2)C2CC2)CCN(c2cnnc(-c3ccccc3O)c2)CC1 ; Cl]

The title compound was prepared as described for the synthesis of Example 2 Step 1 using 1-(6-(2-(methoxymethoxy)phenyl)pyridazin-4-yl)-4-(methoxymethyl)piperidine-4-carboxylic acid (128 mg, 0.37 mmol) and tert-butyl 4-(cyclopropylamino)piperidine-1-carboxylate. LCMS C₂₆H₃₅N₅O₃ requires 565.3, found m/z=564.3 [M+H]⁺.

### Step 2: Synthesis of Title Compound

The title compound was prepared as described for the synthesis of Example 2 Step 2, using N-cyclopropyl-1-(6-(2-hydroxyphenyl)pyridazin-4-yl)-4-(methoxymethyl)-N-(piperidin-4-yl)piperidine-4-carboxamide hydrochloride (79.1 mg, 0.14 mmol) to afford the title compound (8.7 mg, 8% yield) as a white solid.

### Example 60

### N-{1-[2-(1-{4-[(3R)-2,6-DIOXOPIPERIDIN-3-YL]PHENYL}PIPERIDIN-4-YL)ETHYL]PIPERIDIN-4-YL}-1-[6-(2-HYDROXYPHENYL)PYRIDAZIN-4-YL]-N-METHYL-4-(1-METHYL-1H-PYRAZOL-5-YL)PIPERIDINE-4-CARBOXAMIDE

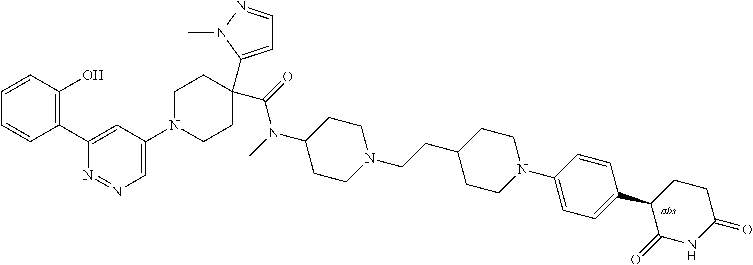 [SMILES: CN(C(=O)C1(c2ccnn2C)CCN(c2cnnc(-c3ccccc3O)c2)CC1)C1CCN(CCC2CCN(c3ccc([C@H]4CCC(=O)NC4=O)cc3)CC2)CC1]

### Step 1: Synthesis of 1-[6-(2-hydroxyphenyl)pyridazin-4-yl]-N-methyl-4-(2-methylpyrazol-3-yl)-N-(piperidin-4-yl)piperidine-4-carboxamide

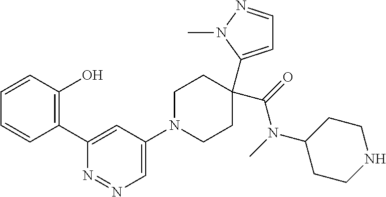 [SMILES: CN(C(=O)C1(c2ccnn2C)CCN(c2cnnc(-c3ccccc3O)c2)CC1)C1CCNCC1]

1-[6-(2-hydroxyphenyl)pyridazin-4-yl]-N-methyl-4-(2-methylpyrazol-3-yl)-N-(piperidin-4-yl)piperidine-4-carboxamide was prepared as described for Example 32, Step 1, using 1-[6-(2-hydroxyphenyl)pyridazin-4-yl]-4-(1-methyl-1H-pyrazol-5-yl)piperidine-4-carboxylic acid. LCMS: C₂₆H₃₃N₇O₂ requires: 475.3, found: m/z=476.4 [M+H]⁺.

### Step 2: Synthesis of the Title Compound

The title compound was prepared as described for Example 1, Step 2, using 1-[6-(2-hydroxyphenyl)pyridazin-4-yl]-N-methyl-4-(2-methylpyrazol-3-yl)-N-(piperidin-4-yl)piperidine-4-carboxamide; trifluoroacetic acid and 2-(1-{4-[(3R)-2,6-dioxopiperidin-3-yl]phenyl}piperidin-4-yl)acetaldehyde to afford N-{1-[2-(1-{4-[(3R)-2,6-dioxopiperidin-3-yl]phenyl}piperidin-4-yl)ethyl]piperidin-4-yl}-1-[6-(2-hydroxyphenyl)pyridazin-4-yl]-N-methyl-4-(1-methyl-1H-pyrazol-5-yl)piperidine-4-carboxamide (2.40 mg, 7.31% yield) as a pale yellow solid. LCMS: C₄₄H₅₅N₉O₄ requires 773.4, found: m/z=774.6 [M+H]⁺.

### Example 61

### N-{1-[2-(1-{4-[(3R)-2,6-DIOXOPIPERIDIN-3-YL]PHENYL}PIPERIDIN-4-YL)ETHYL]PIPERIDIN-4-YL}-4-(1-ETHYL-1H-PYRAZOL-5-YL)-1-[6-(2-HYDROXYPHENYL)PYRIDAZIN-4-YL]-N-METHYLPIPERIDINE-4-CARBOXAMIDE

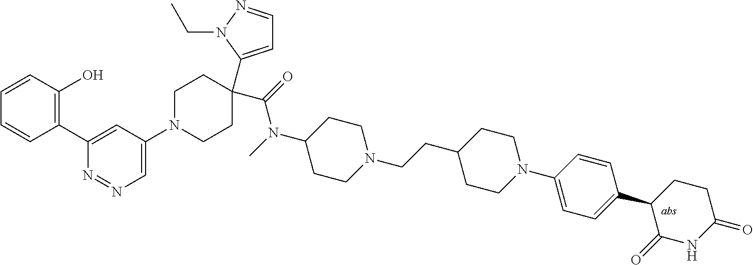 [SMILES: CCn1nccc1C1(C(=O)N(C)C2CCN(CCC3CCN(c4ccc([C@H]5CCC(=O)NC5=O)cc4)CC3)CC2)CCN(c2cnnc(-c3ccccc3O)c2)CC1]

### Step 1: Synthesis of 4-(2-ethylpyrazol-3-yl)-1-[6-(2-hydroxyphenyl)pyridazin-4-yl]-N-methyl-N-(piperidin-4-yl)piperidine-4-carboxamide

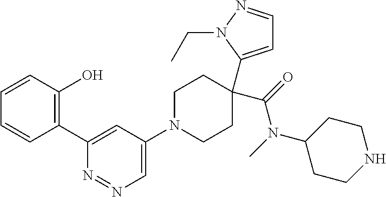 [SMILES: CCn1nccc1C1(C(=O)N(C)C2CCNCC2)CCN(c2cnnc(-c3ccccc3O)c2)CC1]

4-(2-ethylpyrazol-3-yl)-1-[6-(2-hydroxyphenyl)pyridazin-4-yl]-N-methyl-N-(piperidin-4-yl)piperidine-4-carboxamide was prepared as described for Example 32, Step 1, using 4-(1-ethyl-1H-pyrazol-5-yl)-1-(6-(2-(methoxymethoxy)phenyl)pyridazin-4-yl)piperidine-4-carboxylic acid. LCMS: C₂₇H₃₅N₇O₂ requires: 489.3, found: m/z=490.4 [M+H]⁺.

### Step 2: Synthesis of the Title Compound

The title compound was prepared as described for Example 1, Step 2, using 4-(2-ethylpyrazol-3-yl)-1-[6-(2-hydroxyphenyl)pyridazin-4-yl]-N-methyl-N-(piperidin-4-yl)piperidine-4-carboxamide hydrochloride and 2-(1-{4-[(3R)-2,6-dioxopiperidin-3-yl]phenyl}piperidin-4-yl)acetaldehyde to afford N-{1-[2-(1-{4-[(3R)-2,6-dioxopiperidin-3-yl]phenyl}piperidin-4-yl)ethyl]piperidin-4-yl}-4-(1-ethyl-1H-pyrazol-5-yl)-1-[6-(2-hydroxyphenyl)pyridazin-4-yl]-N-methylpiperidine-4-carboxamide (14.7 mg, 60.7% yield) as a yellow solid. LCMS: C₄₅H₅₇N₉O₄ requires 787.5, found: m/z=788.4 [M+H]⁺.

### Example 62

### 4-(1,3-DIMETHYL-1H-PYRAZOL-5-YL)-N-{1-[2-(1-{4-[(3R)-2,6-DIOXOPIPERIDIN-3-YL]PH ENYL}PIPERIDIN-4-YL)ETHYL]PIPERIDIN-4-YL}-1-[6-(2-HYDROXYPHENYL)PYRIDAZIN-4-YL]-N-METHYLPIPERIDINE-4-CARBOXAMIDE

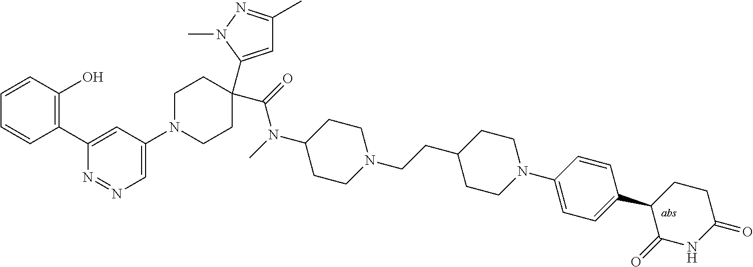 [SMILES: Cc1cc(C2(C(=O)N(C)C3CCN(CCC4CCN(c5ccc([C@H]6CCC(=O)NC6=O)cc5)CC4)CC3)CCN(c3cnnc(-c4ccccc4O)c3)CC2)n(C)n1]

### Step 1: Synthesis of 4-(2,5-dimethylpyrazol-3-yl)-1-[6-(2-hydroxyphenyl)pyridazin-4-yl]-N-methyl-N-(piperidin-4-yl)piperidine-4-carboxamide

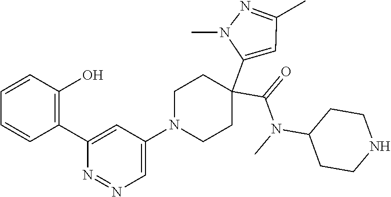 [SMILES: Cc1cc(C2(C(=O)N(C)C3CCNCC3)CCN(c3cnnc(-c4ccccc4O)c3)CC2)n(C)n1]

4-(2,5-dimethylpyrazol-3-yl)-1-[6-(2-hydroxyphenyl)pyridazin-4-yl]-N-methyl-N-(piperidin-4-yl)piperidine-4-carboxamide was prepared as described for Example 32, Step 1, using 4-(1,3-dimethyl-1H-pyrazol-5-yl)-1-(6-(2-(methoxymethoxy)phenyl)pyridazin-4-yl)piperidine-4-carboxylic acid. LCMS: C₂₇H₃₅N₇O₂ requires: 489.3, found: m/z=490.4 [M+H]⁺.

### Step 2: Synthesis of the Title Compound

The title compound was prepared as described for Example 1, Step 2, using 4-(2,5-dimethylpyrazol-3-yl)-1-[6-(2-hydroxyphenyl)pyridazin-4-yl]-N-methyl-N-(piperidin-4-yl)piperidine-4-carboxamide and 2-(1-{4-[(3R)-2,6-dioxopiperidin-3-yl]phenyl}piperidin-4-yl)acetaldehyde to afford 4-(1,3-dimethyl-1H-pyrazol-5-yl)-N-{1-[2-(1-{4-[(3R)-2,6-dioxopiperidin-3-yl]phenyl}piperidin-4-yl)ethyl]piperidin-4-yl}-1-[6-(2-hydroxyphenyl)pyridazin-4-yl]-N-methylpiperidine-4-carboxamide (14.3 mg, 59.1% yield) as a yellow solid. LCMS: C₄₅H₅₇N₉O₄ requires 787.5, found: m/z=788.4 [M+H]⁺.

### Example 63

### N-{1-[(3R)-8-{4-[(3R)-2,6-DIOXOPIPERIDIN-3-YL]PHENYL}-1-OXA-8-AZASPIRO[4.5]DECAN-3-YL]PIPERIDIN-4-YL}-1-[6-(2-HYDROXYPHENYL)PYRIDAZIN-4-YL]-N-METHYL-4-(1-METHYL-1H-PYRAZOL-3-YL)PIPERIDINE-4-CARBOXAMIDE

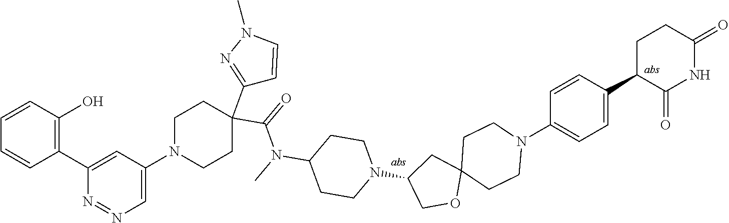 [SMILES: CN(C(=O)C1(c2ccn(C)n2)CCN(c2cnnc(-c3ccccc3O)c2)CC1)C1CCN([C@H]2COC3(CCN(c4ccc([C@H]5CCC(=O)NC5=O)cc4)CC3)C2)CC1]

### Step 1: 1-[6-(2-hydroxyphenyl)pyridazin-4-yl]-N-methyl-4-(1-methylpyrazol-3-yl)-N-(piperidin-4-yl)piperidine-4-carboxamide

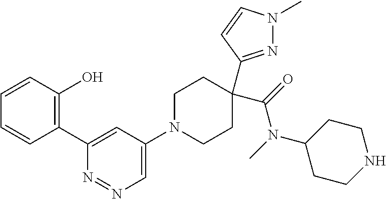 [SMILES: CN(C(=O)C1(c2ccn(C)n2)CCN(c2cnnc(-c3ccccc3O)c2)CC1)C1CCNCC1]

1-[6-(2-hydroxyphenyl)pyridazin-4-yl]-N-methyl-4-(1-methylpyrazol-3-yl)-N-(piperidin-4-yl)piperidine-4-carboxamide was prepared as described for Example 32, Step 1, using 1-(6-(2-hydroxyphenyl)pyridazin-4-yl)-4-(1-methyl-1H-pyrazol-3-yl)piperidine-4-carboxylic acid. LCMS: C₂₆H₃₃N₇O₂ requires: 475.3, found: m/z=476.2 [M+H]⁺.

### Step 2: Synthesis of the Title Compound

The title compound was prepared as described for Example 1, Step 2, using 1-[6-(2-hydroxyphenyl)pyridazin-4-yl]-N-methyl-4-(1-methylpyrazol-3-yl)-N-(piperidin-4-yl)piperidine-4-carboxamide hydrochloride and (3R)-3-(4-{3-oxo-1-oxa-8-azaspiro[4.5]decan-8-yl}phenyl)piperidine-2,6-dione to afford the mixture of isomers. The two isomers were separated by chiral SFC (ChiralPak IH, MeOH+0.1% NH₄OH) and the stereochemistry is arbitrarily assigned. Peak 1 is assigned as N-{1-[(3R)-8-{4-[(3R)-2,6-dioxopiperidin-3-yl]phenyl}-1-oxa-8-azaspiro[4.5]decan-3-yl]piperidin-4-yl}-1-[6-(2-hydroxyphenyl)pyridazin-4-yl]-N-methyl-4-(1-methylpyrazol-3-yl)piperidine-4-carboxamide (31.6 mg, 19.2% yield). LCMS: C₄₅H₅₅N₉O₅ requires 801.4, found: m/z=802.3 [M+H]⁺.

### Example 64

### N-{1-[(3S)-8-{4-[(3R)-2,6-DIOXOPIPERIDIN-3-YL]PHENYL}-1-OXA-8-AZASPIRO[4.5]DECAN-3-YL]PIPERIDIN-4-YL}-1-[6-(2-HYDROXYPHENYL)PYRIDAZIN-4-YL]-N-METHYL-4-(1-METHYL-1H-PYRAZOL-3-YL)PIPERIDINE-4-CARBOXAMIDE

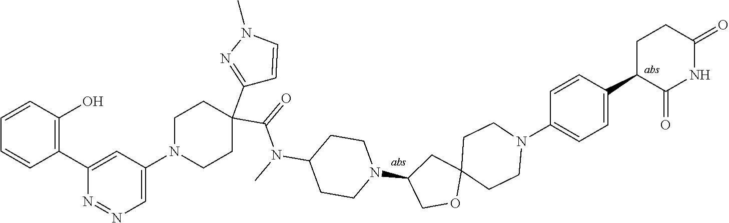 [SMILES: CN(C(=O)C1(c2ccn(C)n2)CCN(c2cnnc(-c3ccccc3O)c2)CC1)C1CCN([C@@H]2COC3(CCN(c4ccc([C@H]5CCC(=O)NC5=O)cc4)CC3)C2)CC1]

The title compound was isolated from Example 63 as peak 2 and was assigned as N-{1-[(3S)-8-{4-[(3R)-2,6-dioxopiperidin-3-yl]phenyl}-1-oxa-8-azaspiro[4.5]decan-3-yl]piperidin-4-yl}-1-[6-(2-hydroxyphenyl)pyridazin-4-yl]-N-methyl-4-(1-methyl-1H-pyrazol-3-yl)piperidine-4-carboxamide (30.8 mg, 18.7% yield). LCMS: C₄₅H₅₅N₉O₅ requires 801.4, found: m/z=802.4 [M+H]⁺

### Example 65

### N-(1-((R)-8-(4-((R)-2,6-DIOXOPIPERIDIN-3-YL)PHENYL)-1-OXA-8-AZASPIRO[4.5]DECAN-3-YL)PIPERIDIN-4-YL)-1-(6-(2-HYDROXYPHENYL)PYRIDAZIN-4-YL)-N-METHYL-4-(THIAZOL-4-YL)PIPERIDINE-4-CARBOXAMIDE

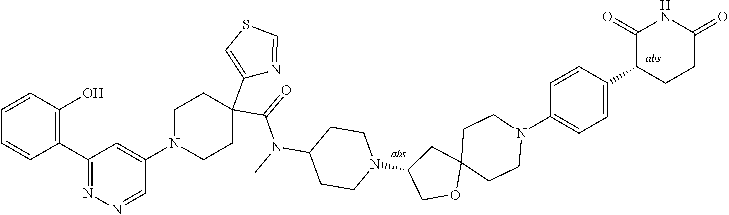 [SMILES: CN(C(=O)C1(c2cscn2)CCN(c2cnnc(-c3ccccc3O)c2)CC1)C1CCN([C@H]2COC3(CCN(c4ccc([C@H]5CCC(=O)NC5=O)cc4)CC3)C2)CC1]

### Step 1: Synthesis of 1-(6-(2-hydroxyphenyl)pyridazin-4-yl)-N-methyl-N-(piperidin-4-yl)-4-(thiazol-4-yl)piperidine-4-carboxamide

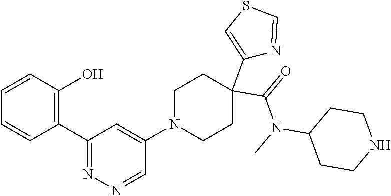 [SMILES: CN(C(=O)C1(c2cscn2)CCN(c2cnnc(-c3ccccc3O)c2)CC1)C1CCNCC1]

1-(6-(2-hydroxyphenyl)pyridazin-4-yl)-N-methyl-N-(piperidin-4-yl)-4-(thiazol-4-yl)piperidine-4-carboxamide was prepared as described for Example 6, Step 1, using 1-(6-(2-(methoxymethoxy)phenyl)pyridazin-4-yl)-4-(thiazol-4-yl)piperidine-4-carboxylic acid and tert-butyl 4-(methylamino)piperidine-1-carboxylate. LCMS: C₂₅H₃₀N₆O₂S requires: 478.2, found: m/z=479.2 [M+H]⁺.

### Step 2: Synthesis of the Title Compound

The title compound was prepared as described for Example 1, Step 2, using 1-(6-(2-hydroxyphenyl)pyridazin-4-yl)-N-methyl-N-(piperidin-4-yl)-4-(thiazol-4-yl)piperidine-4-carboxamide and (R)-3-(4-(3-oxo-1-oxa-8-azaspiro[4.5]decan-8-yl)phenyl)piperidine-2,6-dione to afford N-(1-((RS)-8-(4-((R)-2,6-dioxopiperidin-3-yl)phenyl)-1-oxa-8-azaspiro[4.5]decan-3-yl)piperidin-4-yl)-1-(6-(2-hydroxyphenyl)pyridazin-4-yl)-N-methyl-4-(thiazol-4-yl)piperidine-4-carboxamide. The diastereomers were then separated via chiral SFC purification yield the product (6.10 mg, 7% yield) (Chiralpak IH, peak 2, RT 3.71 min, 99% ee) as a white solid. LCMS: C₄₄H₅₂N₈O₅S requires 804.4, found: m/z=805.3 [M+H]⁺.

### Example 66

### N-(1-((S)-8-(4-((R)-2,6-DIOXOPIPERIDIN-3-YL)PHENYL)-1-OXA-8-AZASPIRO[4.5]DECAN-3-YL)PIPERIDIN-4-YL)-1-(6-(2-HYDROXYPHENYL)PYRIDAZIN-4-YL)-N-METHYL-4-(THIAZOL-4-YL)PIPERIDINE-4-CARBOXAMIDE

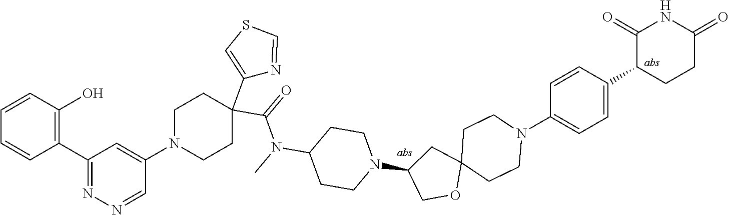 [SMILES: CN(C(=O)C1(c2cscn2)CCN(c2cnnc(-c3ccccc3O)c2)CC1)C1CCN([C@@H]2COC3(CCN(c4ccc([C@H]5CCC(=O)NC5=O)cc4)CC3)C2)CC1]

### Step 1: Synthesis of the Title Compound

The title compound was prepared as described for Example 1, Step 2, using 1-(6-(2-hydroxyphenyl)pyridazin-4-yl)-N-methyl-N-(piperidin-4-yl)-4-(thiazol-4-yl)piperidine-4-carboxamide and (R)-3-(4-(3-oxo-1-oxa-8-azaspiro[4.5]decan-8-yl)phenyl)piperidine-2,6-dione to afford N-(1-((RS)-8-(4-((R)-2,6-dioxopiperidin-3-yl)phenyl)-1-oxa-8-azaspiro[4.5]decan-3-yl)piperidin-4-yl)-1-(6-(2-hydroxyphenyl)pyridazin-4-yl)-N-methyl-4-(thiazol-4-yl)piperidine-4-carboxamide. The diastereomers were then separated via chiral SFC purification yield the product (6.40 mg, 7% yield) (Chiralpak IH, peak 1, RT 3.89 min, 99% ee) to as a white solid. LCMS: C₄₄H₅₂N₈O₅S requires 804.4, found: m/z=805.3 [M+H]⁺.

### Example 67

### N-{1-[(3S)-8-{4-[(3R)-2,6-DIOXOPIPERIDIN-3-YL]PHENYL}-1-OXA-8-AZASPIRO[4.5]DECAN-3-YL]PIPERIDIN-4-YL}-1-[6-(2-HYDROXYPHENYL)PYRIDAZIN-4-YL]-N-METHYL-4-(1-METHYL-1H-PYRAZOL-5-YL)PIPERIDINE-4-CARBOXAMIDE

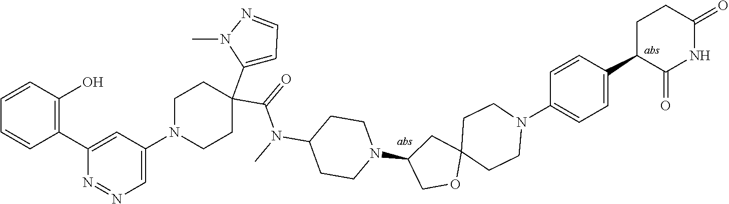 [SMILES: CN(C(=O)C1(c2ccnn2C)CCN(c2cnnc(-c3ccccc3O)c2)CC1)C1CCN([C@@H]2COC3(CCN(c4ccc([C@H]5CCC(=O)NC5=O)cc4)CC3)C2)CC1]

The title compound was prepared as described for Example 1, Step 2, using 1-[6-(2-hydroxyphenyl)pyridazin-4-yl]-N-methyl-4-(2-methylpyrazol-3-yl)-N-(piperidin-4-yl)piperidine-4-carboxamide; trifluoroacetic acid and (3R)-3-(4-{3-oxo-1-oxa-8-azaspiro[4.5]decan-8-yl}phenyl)piperidine-2,6-dione to afford N-{1-[(3RS)-8-{4-[(3R)-2,6-dioxopiperidin-3-yl]phenyl}-1-oxa-8-azaspiro[4.5]decan-3-yl]piperidin-4-yl}-1-[6-(2-hydroxyphenyl)pyridazin-4-yl]-N-methyl-4-(2-methylpyrazol-3-yl)piperidine-4-carboxamide (0.0511 g, 61.71%). The two isomers were separated by chiral SFC (ChiralPak IH, MeOH+0.1% NH₄OH) and the stereochemistry is arbitrarily assigned. Peak 1 is assigned as N-{1-[(3S)-8-{4-[(3R)-2,6-dioxopiperidin-3-yl]phenyl}-1-oxa-8-azaspiro[4.5]decan-3-yl]piperidin-4-yl}-1-[6-(2-hydroxyphenyl)pyridazin-4-yl]-N-methyl-4-(1-methyl-1H-pyrazol-5-yl)piperidine-4-carboxamide (19.1 mg, 35.0% yield). LCMS: C₄₅H₅₅N₉O₅ requires 801.4, found: m/z=802.4 [M+H]⁺.

### Example 68

### N-{1-[(3R)-8-{4-[(3R)-2,6-DIOXOPIPERIDIN-3-YL]PHENYL}-1-OXA-8-AZASPIRO[4.5]DECAN-3-YL]PIPERIDIN-4-YL}-1-[6-(2-HYDROXYPHENYL)PYRIDAZIN-4-YL]-N-METHYL-4-(1-METHYL-1H-PYRAZOL-5-YL)PIPERIDINE-4-CARBOXAMIDE

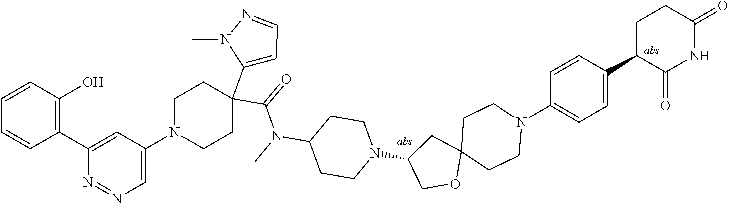 [SMILES: CN(C(=O)C1(c2ccnn2C)CCN(c2cnnc(-c3ccccc3O)c2)CC1)C1CCN([C@H]2COC3(CCN(c4ccc([C@H]5CCC(=O)NC5=O)cc4)CC3)C2)CC1]

The title compound was isolated from Example 67 as peak 2 and was assigned as N-{1-[(3R)-8-{4-[(3R)-2,6-dioxopiperidin-3-yl]phenyl}-1-oxa-8-azaspiro[4.5]decan-3-yl]piperidin-4-yl}-1-[6-(2-hydroxyphenyl)pyridazin-4-yl]-N-methyl-4-(1-methyl-1H-pyrazol-5-yl)piperidine-4-carboxamide (18.7 mg, 34.6% yield). LCMS: C₄₅H₅₅N₉O₅ requires 801.4, found: m/z=802.4 [M+H]⁺.

### Example 69

### RAC-(R)—N-(1-(2-(1-(4-(2,6-DIOXOPIPERIDIN-3-YL)-3-FLUOROPHENYL)PIPERIDIN-4-YL)ETHYL)PIPERIDIN-4-YL)-1-(6-(2-HYDROXYPHENYL)PYRIDAZIN-4-YL)-N-METHYL-4-(THIAZOL-5-YL)PIPERIDINE-4-CARBOXAMIDE

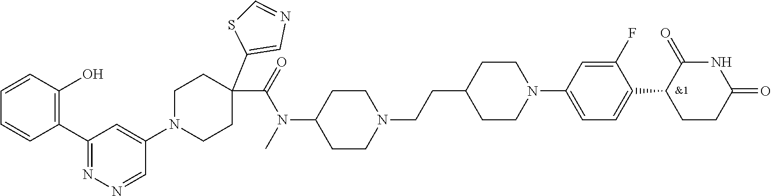 [SMILES: CN(C(=O)C1(c2cncs2)CCN(c2cnnc(-c3ccccc3O)c2)CC1)C1CCN(CCC2CCN(c3ccc([C@H]4CCC(=O)NC4=O)c(F)c3)CC2)CC1]

### Step 1: Synthesis of 1-(6-(2-hydroxyphenyl)pyridazin-4-yl)-N-methyl-N-(piperidin-4-yl)-4-(thiazol-5-yl)piperidine-4-carboxamide hydrochloride

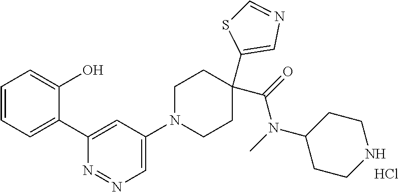 [SMILES: CN(C(=O)C1(c2cncs2)CCN(c2cnnc(-c3ccccc3O)c2)CC1)C1CCNCC1 ; Cl]

This compound was prepared as described for Example 1 Step 1, using lithium 1-(6-(2-hydroxyphenyl)pyridazin-4-yl)-4-(thiazol-5-yl)piperidine-4-carboxylate (53.0 mg, 0.12 mmol) and tert-butyl 4-(methylamino)piperidine-1-carboxylate to afford the desired product as a yellow oil. LCMS C₂₅H₃₀N₆O₂S requires 622.3, found 624.3 [M+H]⁺.

### Step 2: Synthesis of Title Compound

The title compound was prepared as described for Example 1 Step 2, using 1-(6-(2-hydroxyphenyl)pyridazin-4-yl)-N-methyl-N-(piperidin-4-yl)-4-(thiazol-5-yl)piperidine-4-carboxamide hydrochloride (14.2 mg, 0.03 mmol) to afford the title compound (10.3 mg, 41% yield) as a white solid. LCMS C₄₃H₅₁FN₈O₄S requires 794.3, found m/z=795.3 [M+H]⁺.

### Example 70

### (R)—N-(CYCLOPROPYLMETHYL)-N-(1-(2-(1-(4-(2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-4-METHOXYPIPERIDINE-4-CARBOXAMIDE

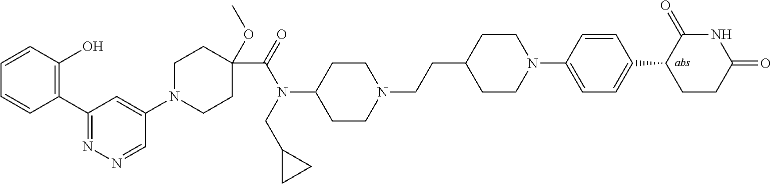 [SMILES: COC1(C(=O)N(CC2CC2)C2CCN(CCC3CCN(c4ccc([C@H]5CCC(=O)NC5=O)cc4)CC3)CC2)CCN(c2cnnc(-c3ccccc3O)c2)CC1]

### Step 1: Synthesis of N-(cyclopropylmethyl)-1-(6-(2-hydroxyphenyl)pyridazin-4-yl)-4-methoxy-N-(piperidin-4-yl)piperidine-4-carboxamide

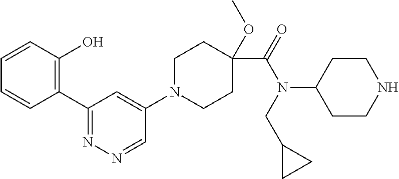 [SMILES: COC1(C(=O)N(CC2CC2)C2CCNCC2)CCN(c2cnnc(-c3ccccc3O)c2)CC1]

To a solution of lithio 4-methoxy-1-{6-[2-(methoxymethoxy)phenyl]pyridazin-4-yl}piperidine-4-carboxylate (200 mg, 0.527 mmol) and tert-butyl 4-[(cyclopropylmethyl)amino]piperidine-1-carboxylate (201 mg, 0.791 mmol) in DCM (2.00 mL) was added N,N-diisopropylethylamine (0.37 mL, 272 mg, 2.11 mmol). Then bis(2-oxo-1,3-oxazolidin-3-yl)phosphinoyl chloride (402 mg, 1.58 mmol) was added and the reaction mixture was stirred at 40° C. Then, the reaction was quenched with water, extracted with DCM, dried over Na₂SO₄, filtered and the solvent removed under reduced pressure. The crude material was purified via chromatography (MeOH:DCM, gradient) to yield the desired product as an orange foam. The material was dissolved in 10 mL methanol and 4M HCl in dioxane (10.0 mL) was added in one portion. The reaction was stirred at RT for 1 hour and then concentrated in vacuo to yield the desired product (220 mg, 74%) as a white solid. LCMS: C₂₆H₃₅N₅O₃ requires: 465.2, found: m/z=466.2 [M+H]⁺.

### Step 2: Synthesis of the Title Compound

N-(cyclopropylmethyl)-1-[6-(2-hydroxyphenyl)pyridazin-4-yl]-4-methoxy-N-(piperidin-4-yl)piperidine-4-carboxamide hydrochloride (50.0 mg, 0.099 mmol) was dissolved in MeCN (0.45 mL) and 2-(1-{4-[(3R)-2,6-dioxopiperidin-3-yl]phenyl}piperidin-4-yl)acetaldehyde (34.4 mg, 0.109 mmol) was added in one portion. The flask was agitated and then MeOH (0.13 mL) was slowly added until all the solids have dissolved. Sodium triacetoxyborohydride (63.32 mg, 0.2988 mmol) was then added in one portion and the reaction was stirred at RT for 30 mins. After 30 mins, the reaction was checked by LCMS and the reaction mixture was dissolved in DCM and water was added. The reaction was extracted 3× with DCM and concentrated in vacuo. The residue was then taken up in 1:1 ACN:H₂O and purified via RP-HPLC to afford the desired product (44.0 mg, 52.1%) as a white solid. LCMS: C₄₄H₅₇N₇O₅ requires: 763.4, found: m/z=764.4 [M+H]⁺.

### Example 71

### N-{1-[(3RS)-8-{4-[(3R)-2,6-DIOXOPIPERIDIN-3-YL]PHENYL}-1-OXA-8-AZASPIRO[4.5]DECAN-3-YL]PIPERIDIN-4-YL}-4-(1-ETHYL-1H-PYRAZOL-5-YL)-1-[6-(2-HYDROXYPHENYL)PYRIDAZIN-4-YL]-N-METHYLPIPERIDINE-4-CARBOXAMIDE

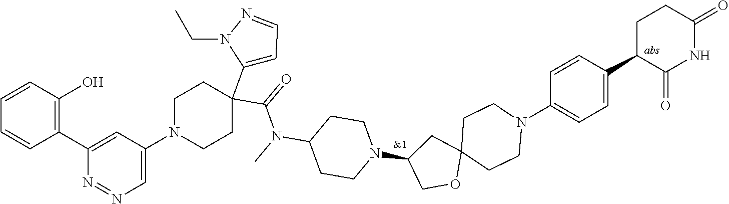 [SMILES: CCn1nccc1C1(C(=O)N(C)C2CCN([C@@H]3COC4(CCN(c5ccc([C@H]6CCC(=O)NC6=O)cc5)CC4)C3)CC2)CCN(c2cnnc(-c3ccccc3O)c2)CC1]

The title compound was prepared as described for Example 1, Step 2, using 4-(2-ethylpyrazol-3-yl)-1-[6-(2-hydroxyphenyl)pyridazin-4-yl]-N-methyl-N-(piperidin-4-yl)piperidine-4-carboxamide hydrochloride and (3R)-3-(4-{3-oxo-1-oxa-8-azaspiro[4.5]decan-8-yl}phenyl)piperidine-2,6-dione to afford N-{1-[(3RS)-8-{4-[(3R)-2,6-dioxopiperidin-3-yl]phenyl}-1-oxa-8-azaspiro[4.5]decan-3-yl]piperidin-4-yl}-4-(1-ethyl-1H-pyrazol-5-yl)-1-[6-(2-hydroxyphenyl)pyridazin-4-yl]-N-methylpiperidine-4-carboxamide (15.3 mg, 53.9%). LCMS: C₄₆H₅₇N₉O₅ requires 815.4, found: m/z=816.3 [M+H]⁺.

### Example 72

### 4-(1,3-DIMETHYL-1H-PYRAZOL-5-YL)-N-{1-[(3RS)-8-{4-[(3R)-2,6-DIOXOPIPERIDIN-3-YL]PHENYL}-1-OXA-8-AZASPIRO[4.5]DECAN-3-YL]PIPERIDIN-4-YL}-1-[6-(2-HYDROXYPHENYL)PYRIDAZIN-4-YL]-N-METHYLPIPERIDINE-4-CARBOXAMIDE

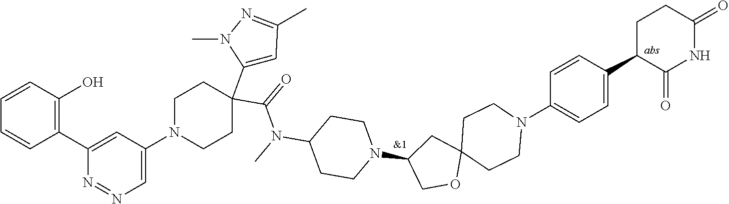 [SMILES: Cc1cc(C2(C(=O)N(C)C3CCN([C@@H]4COC5(CCN(c6ccc([C@H]7CCC(=O)NC7=O)cc6)CC5)C4)CC3)CCN(c3cnnc(-c4ccccc4O)c3)CC2)n(C)n1]

The title compound was prepared as described for Example 1, Step 2, using 4-(2,5-dimethylpyrazol-3-yl)-1-[6-(2-hydroxyphenyl)pyridazin-4-yl]-N-methyl-N-(piperidin-4-yl)piperidine-4-carboxamide hydrochloride and (3R)-3-(4-{3-oxo-1-oxa-8-azaspiro[4.5]decan-8-yl}phenyl)piperidine-2,6-dione to afford 4-(1,3-dimethyl-1H-pyrazol-5-yl)-N-{1-[(3RS)-8-{4-[(3R)-2,6-dioxopiperidin-3-yl]phenyl}-1-oxa-8-azaspiro[4.5]decan-3-yl]piperidin-4-yl}-1-[6-(2-hydroxyphenyl)pyridazin-4-yl]-N-methylpiperidine-4-carboxamide (14.6 mg, 51.6%). LCMS: C₄₆H₅₇N₉O₅ requires 815.4, found: m/z=816.3 [M+H]⁺.

### Example 73

### N-{1-[(3RS)-8-{4-[(3R)-2,6-DIOXOPIPERIDIN-3-YL]PHENYL}-1-OXA-8-AZASPIRO[4.5]DECAN-3-YL]PIPERIDIN-4-YL}-1-[6-(2-HYDROXYPHENYL)PYRIDAZIN-4-YL]-N-METHYL-4-[1-(OXAN-4-YL)-1H-PYRAZOL-5-YL]PIPERIDINE-4-CARBOXAMIDE

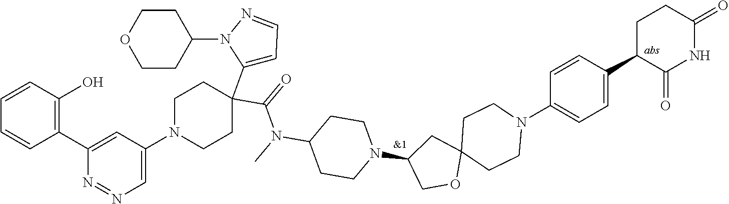 [SMILES: CN(C(=O)C1(c2ccnn2C2CCOCC2)CCN(c2cnnc(-c3ccccc3O)c2)CC1)C1CCN([C@@H]2COC3(CCN(c4ccc([C@H]5CCC(=O)NC5=O)cc4)CC3)C2)CC1]

### Step 1: 1-[6-(2-hydroxyphenyl)pyridazin-4-yl]-N-methyl-4-[2-(oxan-4-yl)pyrazol-3-yl]-N-(piperidin-4-yl)piperidine-4-carboxamide

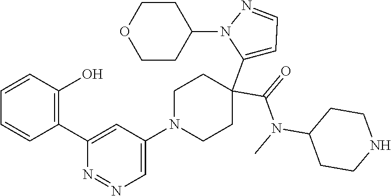 [SMILES: CN(C(=O)C1(c2ccnn2C2CCOCC2)CCN(c2cnnc(-c3ccccc3O)c2)CC1)C1CCNCC1]

1-[6-(2-hydroxyphenyl)pyridazin-4-yl]-N-methyl-4-[2-(oxan-4-yl)pyrazol-3-yl]-N-(piperidin-4-yl)piperidine-4-carboxamide was prepared as described for Example 32, Step 1, using 1-(6-(2-(methoxymethoxy)phenyl)pyridazin-4-yl)-4-(1-(tetrahydro-2H-pyran-4-yl)-1H-pyrazol-5-yl)piperidine-4-carboxylic acid. LCMS: C₃₀H₃₉N₇O₃ requires: 545.3, found: m/z=546.4 [M+H]⁺.

### Step 2: Synthesis of the Title Compound

The title compound was prepared as described for Example 1, Step 2, using 1-[6-(2-hydroxyphenyl)pyridazin-4-yl]-N-methyl-4-[2-(oxan-4-yl)pyrazol-3-yl]-N-(piperidin-4-yl)piperidine-4-carboxamide hydrochloride and (3R)-3-(4-{3-oxo-1-oxa-8-azaspiro[4.5]decan-8-yl}phenyl)piperidine-2,6-dione to afford N-{1-[(3RS)-8-{4-[(3R)-2,6-dioxopiperidin-3-yl]phenyl}-1-oxa-8-azaspiro[4.5]decan-3-yl]piperidin-4-yl}-1-[6-(2-hydroxyphenyl)pyridazin-4-yl]-N-methyl-4-[2-(oxan-4-yl)pyrazol-3-yl]piperidine-4-carboxamide (11.2 mg, 44.1%). LCMS: C₄₉H₆₁N₉O₆ requires 871.5, found: m/z=872.4 [M+H]⁺.

### Example 74

### N-{1-[(3RS)-8-{4-[(3R)-2,6-DIOXOPIPERIDIN-3-YL]PHENYL}-1-OXA-8-AZASPIRO[4.5]DECAN-3-YL]PIPERIDIN-4-YL}-1-[6-(2-HYDROXYPHENYL)PYRIDAZIN-4-YL]-N-METHYL-4-[1-METHYL-3-(TRIFLUOROMETHYL)-1H-PYRAZOL-5-YL]PIPERIDINE-4-CARBOXAMIDE

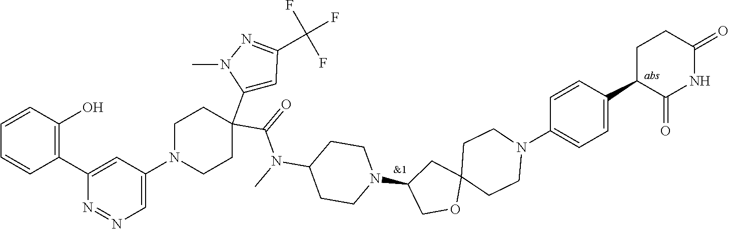 [SMILES: CN(C(=O)C1(c2cc(C(F)(F)F)nn2C)CCN(c2cnnc(-c3ccccc3O)c2)CC1)C1CCN([C@@H]2COC3(CCN(c4ccc([C@H]5CCC(=O)NC5=O)cc4)CC3)C2)CC1]

### Step 1: 1-[6-(2-hydroxyphenyl)pyridazin-4-yl]-N-methyl-4-[2-methyl-5-(trifluoromethyl)pyrazol-3-yl]-N-(piperidin-4-yl)piperidine-4-carboxamide

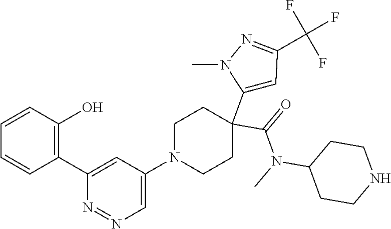 [SMILES: CN(C(=O)C1(c2cc(C(F)(F)F)nn2C)CCN(c2cnnc(-c3ccccc3O)c2)CC1)C1CCNCC1]

1-[6-(2-hydroxyphenyl)pyridazin-4-yl]-N-methyl-4-[2-methyl-5-(trifluoromethyl)pyrazol-3-yl]-N-(piperidin-4-yl)piperidine-4-carboxamide was prepared as described for Example 32, Step 1, using 1-(6-(2-(methoxymethoxy)phenyl)pyridazin-4-yl)-4-(1-methyl-3-(trifluoromethyl)-1H-pyrazol-5-yl)piperidine-4-carboxylic acid. LCMS: C₂₇H₃₂F₃N₇O₂ requires: 543.3, found: m/z=544.4 [M+H]⁺.

### Step 2: Synthesis of the Title Compound

The title compound was prepared as described for Example 1, Step 2, using 1-[6-(2-hydroxyphenyl)pyridazin-4-yl]-N-methyl-4-[2-methyl-5-(trifluoromethyl)pyrazol-3-yl]-N-(piperidin-4-yl)piperidine-4-carboxamide hydrochloride and (3R)-3-(4-{3-oxo-1-oxa-8-azaspiro[4.5]decan-8-yl}phenyl)piperidine-2,6-dione to afford N-{1-[(3RS)-8-{4-[(3R)-2,6-dioxopiperidin-3-yl]phenyl}-1-oxa-8-azaspiro[4.5]decan-3-yl]piperidin-4-yl}-1-[6-(2-hydroxyphenyl)pyridazin-4-yl]-N-methyl-4-[2-methyl-5-(trifluoromethyl)pyrazol-3-yl]piperidine-4-carboxamide (9.6 mg, 37.3%). LCMS: C₄₆H₅₄F₃N₉O₅ requires 869.4, found: m/z=870.3 [M+H]⁺.

### Example 75

### 4-(1,4-DIMETHYL-1H-PYRAZOL-5-YL)-N-{1-[(3RS)-8-{4-[(3R)-2,6-DIOXOPIPERIDIN-3-YL]PHENYL}-1-OXA-8-AZASPIRO[4.5]DECAN-3-YL]PIPERIDIN-4-YL}-1-[6-(2-HYDROXYPHENYL)PYRIDAZIN-4-YL]-N-METHYLPIPERIDINE-4-CARBOXAMIDE

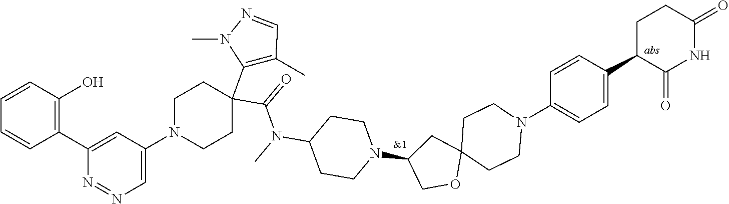 [SMILES: Cc1cnn(C)c1C1(C(=O)N(C)C2CCN([C@@H]3COC4(CCN(c5ccc([C@H]6CCC(=O)NC6=O)cc5)CC4)C3)CC2)CCN(c2cnnc(-c3ccccc3O)c2)CC1]

### Step 1: 4-(2,4-dimethylpyrazol-3-yl)-1-[6-(2-hydroxyphenyl)pyridazin-4-yl]-N-methyl-N-(piperidin-4-yl)piperidine-4-carboxamide

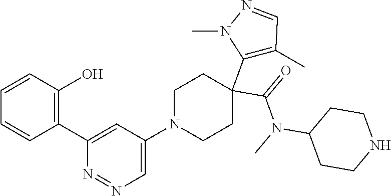 [SMILES: Cc1cnn(C)c1C1(C(=O)N(C)C2CCNCC2)CCN(c2cnnc(-c3ccccc3O)c2)CC1]

4-(2,4-dimethylpyrazol-3-yl)-1-[6-(2-hydroxyphenyl)pyridazin-4-yl]-N-methyl-N-(piperidin-4-yl)piperidine-4-carboxamide was prepared as described for Example 32, Step 1, using 4-(1,4-dimethyl-1H-pyrazol-5-yl)-1-(6-(2-(methoxymethoxy)phenyl)pyridazin-4-yl)piperidine-4-carboxylic acid. LCMS: C₂₇H₃₅N₇O₂ requires: 489.3, found: m/z=490.4 [M+H]⁺.

### Step 2: Synthesis of the Title Compound

The title compound was prepared as described for Example 1, Step 2, using 4-(2,4-dimethylpyrazol-3-yl)-1-[6-(2-hydroxyphenyl)pyridazin-4-yl]-N-methyl-N-(piperidin-4-yl)piperidine-4-carboxamide hydrochloride and (3R)-3-(4-{3-oxo-1-oxa-8-azaspiro[4.5]decan-8-yl}phenyl)piperidine-2,6-dione to afford 4-(2,4-dimethylpyrazol-3-yl)-N-{1-[(3RS)-8-{4-[(3R)-2,6-dioxopiperidin-3-yl]phenyl}-1-oxa-8-azaspiro[4.5]decan-3-yl]piperidin-4-yl}-1-[6-(2-hydroxyphenyl)pyridazin-4-yl]-N-methylpiperidine-4-carboxamide (7.4 mg, 30.5%). LCMS: C₄₆H₅₇N₉O₅ requires 815.4, found: m/z=816.4 [M+H]⁺.

### Example 76

### N-{1-[(3RS)-8-{4-[(3R)-2,6-DIOXOPIPERIDIN-3-YL]PHENYL}-1-OXA-8-AZASPIRO[4.5]DECAN-3-YL]PIPERIDIN-4-YL}-1-[6-(2-HYDROXYPHENYL)PYRIDAZIN-4-YL]-N-METHYL-4-(2-METHYL-2H-INDAZOL-3-YL)PIPERIDINE-4-CARBOXAMIDE

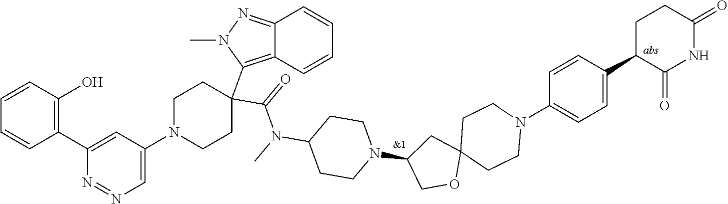 [SMILES: CN(C(=O)C1(c2c3ccccc3nn2C)CCN(c2cnnc(-c3ccccc3O)c2)CC1)C1CCN([C@@H]2COC3(CCN(c4ccc([C@H]5CCC(=O)NC5=O)cc4)CC3)C2)CC1]

### Step 1: 1-[6-(2-hydroxyphenyl)pyridazin-4-yl]-N-methyl-4-(2-methylindazol-3-yl)-N-(piperidin-4-yl)piperidine-4-carboxamide

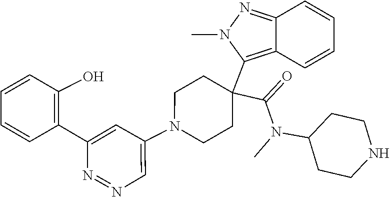 [SMILES: CN(C(=O)C1(c2c3ccccc3nn2C)CCN(c2cnnc(-c3ccccc3O)c2)CC1)C1CCNCC1]

1-[6-(2-hydroxyphenyl)pyridazin-4-yl]-N-methyl-4-(2-methylindazol-3-yl)-N-(piperidin-4-yl)piperidine-4-carboxamide was prepared as described for Example 32, Step 1, using 1-(6-(2-(methoxymethoxy)phenyl)pyridazin-4-yl)-4-(2-methyl-2H-indazol-3-yl)piperidine-4-carboxylic acid. LCMS: C₃₀H₃₅N₇O₂ requires: 525.3, found: m/z=526.4 [M+H]⁺.

### Step 2: Synthesis of the Title Compound

The title compound was prepared as described for Example 1, Step 2, using 1-[6-(2-hydroxyphenyl)pyridazin-4-yl]-N-methyl-4-(2-methylindazol-3-yl)-N-(piperidin-4-yl)piperidine-4-carboxamide hydrochloride and (3R)-3-(4-{3-oxo-1-oxa-8-azaspiro[4.5]decan-8-yl}phenyl)piperidine-2,6-dione to afford N-{1-[(3RS)-8-{4-[(3R)-2,6-dioxopiperidin-3-yl]phenyl}-1-oxa-8-azaspiro[4.5]decan-3-yl]piperidin-4-yl}-1-[6-(2-hydroxyphenyl)pyridazin-4-yl]-N-methyl-4-(2-methylindazol-3-yl)piperidine-4-carboxamide (12.5 mg, 51.6%). LCMS: C₄₉H₅₇N₉O₅ requires 851.4, found: m/z=852.4 [M+H]⁺.

### Example 77A AND 77B

### (S)—N-(1-(2-(1-(4-((R)-2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-3-METHOXY-N-METHYLPYRROLIDINE-3-CARBOXAMIDE AND (R)—N-(1-(2-(1-(4-((S)-2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-3-METHOXY-N-METHYLPYRROLIDINE-3-CARBOXAMIDE

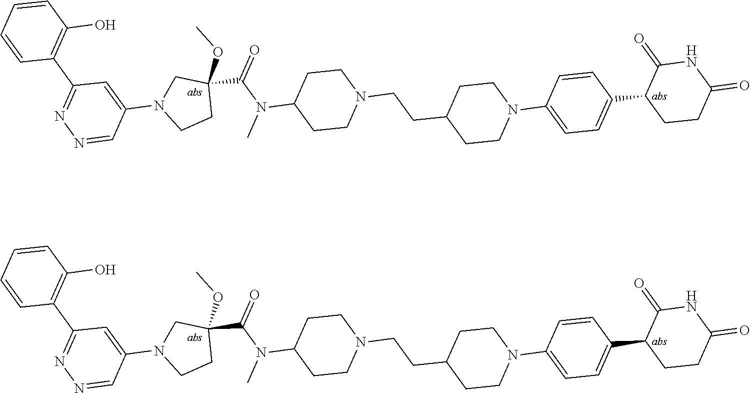 [SMILES: CO[C@@]1(C(=O)N(C)C2CCN(CCC3CCN(c4ccc([C@H]5CCC(=O)NC5=O)cc4)CC3)CC2)CCN(c2cnnc(-c3ccccc3O)c2)C1 ; CO[C@]1(C(=O)N(C)C2CCN(CCC3CCN(c4ccc([C@@H]5CCC(=O)NC5=O)cc4)CC3)CC2)CCN(c2cnnc(-c3ccccc3O)c2)C1]

(RS)—N-(1-(2-(1-(4-((R)-2,6-dioxopiperidin-3-yl)phenyl)piperidin-4-yl)ethyl)piperidin-4-yl)-1-(6-(2-hydroxyphenyl)pyridazin-4-yl)-3-methoxy-N-methylpyrrolidine-3-carboxamide (Example 55) was subjected to two stages of SFC chromatography (Chiralpak IH, EtOH+60ACN+0.1% NH₄OH followed by Whelk-O1 (R,R), EtOH+60ACN+0.1% NH₄OH) to afford Example 77A (second peak in second SFC stage, RT=2.47 min, 36.7 mg, 28% yield) and Example 77B (second peak in second SFC stage, RT=3.28 min, 33.6 mg, 26% yield) as white solids. Example 77A LCMS C₄₀H₅₁N₇O₅ requires: 709.4, found: m/z=710.4 [M+H]⁺. Example 77B LCMS C₄₀H₅₁N₇O₅ requires: 709.4, found: m/z=710.4 [M+H]⁺. Assignment of absolute stereochemistry for both compounds is arbitrary. Two additional isomers were also isolated following a similar process.

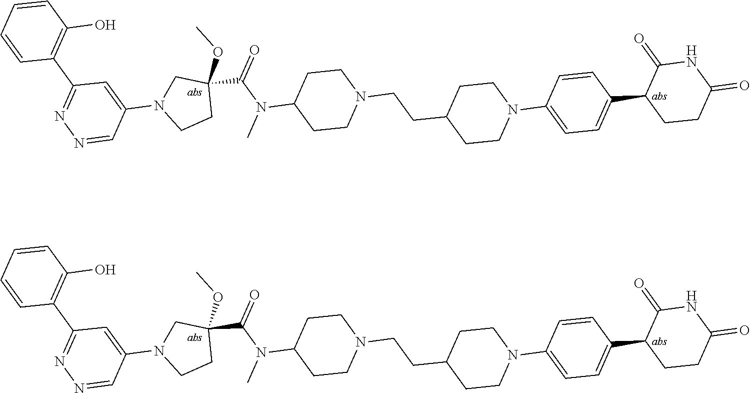 [SMILES: CO[C@@]1(C(=O)N(C)C2CCN(CCC3CCN(c4ccc([C@@H]5CCC(=O)NC5=O)cc4)CC3)CC2)CCN(c2cnnc(-c3ccccc3O)c2)C1 ; CO[C@]1(C(=O)N(C)C2CCN(CCC3CCN(c4ccc([C@@H]5CCC(=O)NC5=O)cc4)CC3)CC2)CCN(c2cnnc(-c3ccccc3O)c2)C1]

### Example 78

### (R)-3-(4-(4-(2-(9-((RS)-1-(6-(2-HYDROXYPHENYL)PYRIDAZIN-4-YL)-3-METHOXYPYRROLIDINE-3-CARBONYL)-3,9-DIAZASPIRO[5.5]UNDECAN-3-YL)ETHYL)PIPERIDIN-1-YL)PHENYL)PIPERIDINE-2,6-DIONE

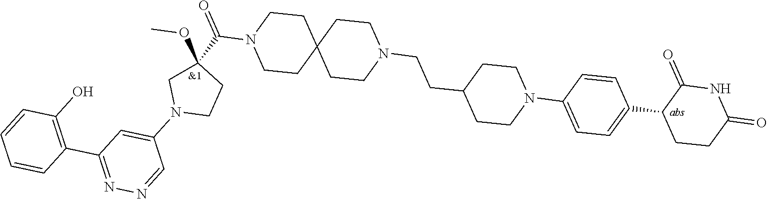 [SMILES: CO[C@@]1(C(=O)N2CCC3(CCN(CCC4CCN(c5ccc([C@H]6CCC(=O)NC6=O)cc5)CC4)CC3)CC2)CCN(c2cnnc(-c3ccccc3O)c2)C1]

### Step 1: rac-(R)-(1-(6-(2-hydroxyphenyl)pyridazin-4-yl)-3-methoxypyrrolidin-3-yl)(3,9-diazaspiro[5.5]undecan-3-yl)methanone hydrochloride

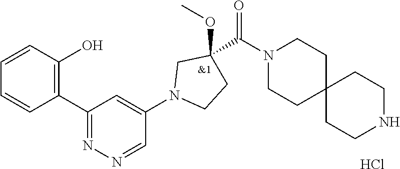 [SMILES: CO[C@@]1(C(=O)N2CCC3(CCNCC3)CC2)CCN(c2cnnc(-c3ccccc3O)c2)C1 ; Cl]

The title compound was prepared as described for Example 1, Step 1 using rac-(R)-3-methoxy-1-(6-(2-(methoxymethoxy)phenyl)pyridazin-4-yl)pyrrolidine-3-carboxylic acid (Intermediate 5) and tert-butyl 3,9-diazaspiro[5.5]undecane-3-carboxylate to afford the product as a yellow oil. LCMS C₂₅H₃₃N₅O₃ requires 451.3, found m/z=452.3 [M+H]⁺.

### Step 2: Synthesis of Title Compound

The title compound was prepared as described for Example 1, Step 2, using rac-(R)-(1-(6-(2-hydroxyphenyl)pyridazin-4-yl)-3-methoxypyrrolidin-3-yl)(3,9-diazaspiro[5.5]undecan-3-yl)methanone hydrochloride to afford the title compound (24.9 mgs, 24% yield) as a white solid. LCMS: C₄₃H₅₅N₇O₅ requires: 749.4, found: m/z=750.4 [M+H]⁺.

### Example 79

### RAC-(R)-3-(2-FLUORO-4-(4-(2-(2-((S)-1-(6-(2-HYDROXYPHENYL)PYRIDAZIN-4-YL)-3-METHOXYPYRROLIDINE-3-CARBONYL)-2,7-DIAZASPIRO[3.5]NONAN-7-YL)ETHYL)PIPERIDIN-1-YL)PHENYL)PIPERIDINE-2,6-DIONE

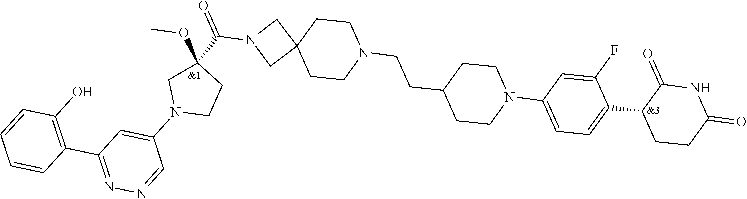 [SMILES: CO[C@@]1(C(=O)N2CC3(CCN(CCC4CCN(c5ccc([C@H]6CCC(=O)NC6=O)c(F)c5)CC4)CC3)C2)CCN(c2cnnc(-c3ccccc3O)c2)C1]

### Step 1: Synthesis of rac-(R)-(1-(6-(2-hydroxyphenyl)pyridazin-4-yl)-3-methoxypyrrolidin-3-yl)(2,7-diazaspiro[3.5]nonan-2-yl)methanone (TFA salt)

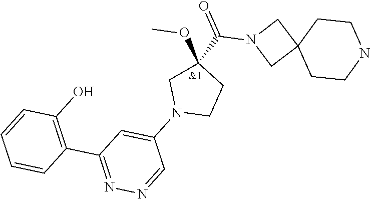 [SMILES: CO[C@@]1(C(=O)N2CC3(CCNCC3)C2)CCN(c2cnnc(-c3ccccc3O)c2)C1]

The title compound was prepared as described for Example 1, Step 1, using rac-(R)-3-methoxy-1-(6-(2-(methoxymethoxy)phenyl)pyridazin-4-yl)pyrrolidine-3-carboxylic acid (Intermediate 5) and tert-butyl 2,7-diazaspiro[3.5]nonane-7-carboxylate. TFA in DCM was used for the cleavage of the BOC protecting group. The product was afforded as yellow oil. LCMS C₂₃H₂₉N₅O₃ requires 423.2, found m/z=424.2. [M+H]⁺.

### Step 2: Synthesis of Title Compound

The title compound was prepared as described for Example 1, Step 2, using rac-(R)-(1-(6-(2-hydroxyphenyl)pyridazin-4-yl)-3-methoxypyrrolidin-3-yl)(2,7-diazaspiro[3.5]nonan-2-yl)methanone 2,2,2-trifluoroacetate to afford the title compound (22.3 mg, 18% yield) as a white solid. LCMS: C₄₁H₅₀FN₇O₅ requires: 739.4, found: m/z=740.4 [M+H]⁺.

### Example 80A

### (R)—N-(1-(2-(1-(4-((R)-2,6-DIOXOPIPERIDIN-3-YL)PHENYL)PIPERIDIN-4-YL)ETHYL)PIPERIDIN-4-YL)-3-ETHOXY-1-(6-(2-HYDROXYPHENYL)PYRIDAZIN-4-YL)-N-METHYLPYRROLIDINE-3-CARBOXAMIDE

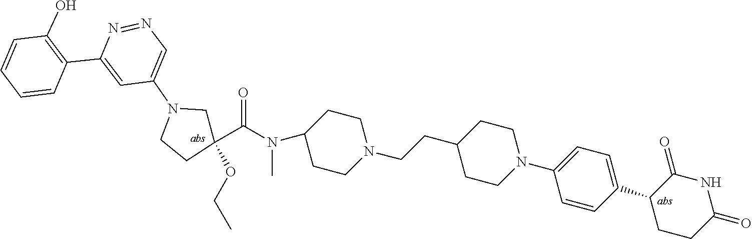 [SMILES: CCO[C@]1(C(=O)N(C)C2CCN(CCC3CCN(c4ccc([C@H]5CCC(=O)NC5=O)cc4)CC3)CC2)CCN(c2cnnc(-c3ccccc3O)c2)C1]

### Step 1: Synthesis of rac-(R)-3-ethoxy-1-(6-(2-hydroxyphenyl)pyridazin-4-yl)-N-methyl-N-(piperidin-4-yl)pyrrolidine-3-carboxamide (TFA salt)

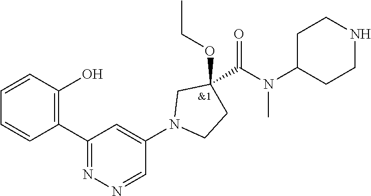 [SMILES: CCO[C@@]1(C(=O)N(C)C2CCNCC2)CCN(c2cnnc(-c3ccccc3O)c2)C1]

The title compound was prepared as described for Example 2, Step 2 using rac-(R)-3-ethoxy-1-(6-(2-(methoxymethoxy)phenyl)pyridazin-4-yl)pyrrolidine-3-carboxylic acid (Intermediate 26) and tert-butyl 4-(methylamino)piperidine-1-carboxylate. TFA in DCM was used for cleavage of the BOC protecting group. The desired product was afforded as a yellow oil. LCMS C₂₃H₃₁N₅O₃ requires 425.2, found m/z=426.2 [M+H]⁺.

### Step 2: Synthesis of the Title Compound

The title compound was synthesized as prepared for Example 1, Step 2, using rac-(R)-3-ethoxy-1-(6-(2-hydroxyphenyl)pyridazin-4-yl)-N-methyl-N-(piperidin-4-yl)pyrrolidine-3-carboxamide 2,2,2-trifluoroacetate. After RP-HPLC, this compound was subjected to 2 stages of chiral SFC purification (Whelk-O1 (R,R), EtOH+60ACN+0.1% NH₄OH followed by Chiralpak IBN, EtOH+60ACN+0.1% NH₄OH). The peak was collected and concentrated to afford the title compound as a white solid (Peak 1 after second SFC stage, RT=7.33 min, 26.4 mg, 38% yield). Assignment of absolute stereochemistry is arbitrary. LCMS C₄₁H₅₃N₇O₅ requires 723.4, found m/z=724.4 [M+H]⁺.

### Example 80B

### (S)—N-(1-(2-(1-(4-((R)-2,6-DIOXOPIPERIDIN-3-YL)PHENYL)PIPERIDIN-4-YL)ETHYL)PIPERIDIN-4-YL)-3-ETHOXY-1-(6-(2-HYDROXYPHENYL)PYRIDAZIN-4-YL)-N-METHYLPYRROLIDINE-3-CARBOXAMIDE

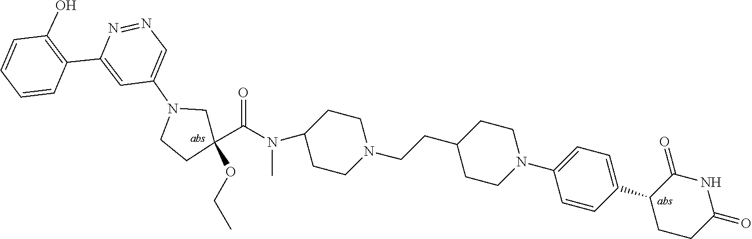 [SMILES: CCO[C@@]1(C(=O)N(C)C2CCN(CCC3CCN(c4ccc([C@H]5CCC(=O)NC5=O)cc4)CC3)CC2)CCN(c2cnnc(-c3ccccc3O)c2)C1]

### Step 1: Synthesis of the Title Compound

The title compound was synthesized as prepared for Example 1, Step 2, using rac-(R)-3-ethoxy-1-(6-(2-hydroxyphenyl)pyridazin-4-yl)-N-methyl-N-(piperidin-4-yl)pyrrolidine-3-carboxamide 2,2,2-trifluoroacetate. After RP-HPLC, this compound was subjected to 2 stages of chiral SFC purification (Whelk-O1 (R,R), EtOH+60ACN+0.1% NH₄OH followed by Chiralpak IBN, EtOH+60ACN+0.1% NH₄OH). The peak was collected and concentrated to afford the title compound as a white solid (Peak 2 after second SFC stage, RT=7.55 min, 22.1 mg, 31.6% yield). Assignment of absolute stereochemistry is arbitrary. LCMS C₄₁H₅₃N₇O₅ requires 723.4, found m/z=724.4 [M+H]⁺.

### Example 81

### (R)—N-(1-(2-(1-(4-((S)-2,6-DIOXOPIPERIDIN-3-YL)-3-FLUOROPHENYL)PIPERIDIN-4-YL)ETHYL)PIPERIDIN-4-YL)-1-(6-(2-HYDROXYPHENYL)PYRIDAZIN-4-YL)-3-METHOXY-N-METHYLPYRROLIDINE-3-CARBOXAMIDE

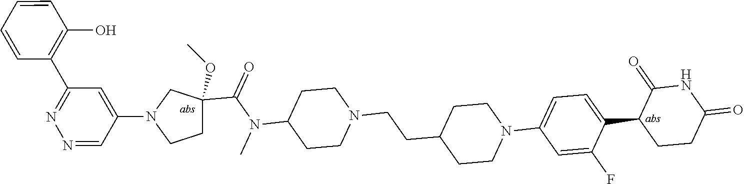 [SMILES: CO[C@]1(C(=O)N(C)C2CCN(CCC3CCN(c4ccc([C@@H]5CCC(=O)NC5=O)c(F)c4)CC3)CC2)CCN(c2cnnc(-c3ccccc3O)c2)C1]

### Step 1: Synthesis of Title Compound

The title compound was prepared as described for Example 1, Step 2, using rac-(R)-1-(6-(2-hydroxyphenyl)pyridazin-4-yl)-3-methoxy-N-methyl-N-(piperidin-4-yl)pyrrolidine-3-carboxamide hydrochloride (Example 55, Step 1) and rac-(R)-2-(1-(4-(2,6-dioxopiperidin-3-yl)-3-fluorophenyl)piperidin-4-yl)acetaldehyde. After RP-HPLC, this compound was subjected to 2 stages of chiral SFC purification (Chiralpak IH, EtOH+60ACN+0.1% NH₄OH followed by Whelk-O1 (R,R), EtOH+60ACN+0.1% NH₄OH). The peak was collected and concentrated to afford the title compound as a white solid (Peak 2 after second SFC stage, RT=7.52 min, 21.4 mg, 110% yield). Assignment of absolute stereochemistry is arbitrary. LCMS C₄₀H₅₀FN₇O₅ requires, 727.4, found m/z=728.4 [M+H]⁺.

### Example 82

### (R)—N-(1-(2-(1-(4-((S)-2,6-DIOXOPIPERIDIN-3-YL)-3,5-DIFLUOROPHENYL)PIPERIDIN-4-YL)ETHYL)PIPERIDIN-4-YL)-1-(6-(2-HYDROXYPHENYL)PYRIDAZIN-4-YL)-3-METHOXY-N-METHYLPYRROLIDINE-3-CARBOXAMIDE

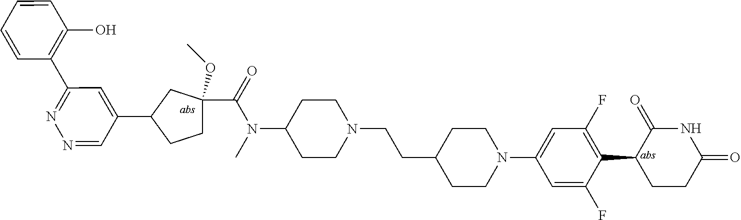 [SMILES: CO[C@]1(C(=O)N(C)C2CCN(CCC3CCN(c4cc(F)c([C@@H]5CCC(=O)NC5=O)c(F)c4)CC3)CC2)CCC(c2cnnc(-c3ccccc3O)c2)C1]

### Step 1: Synthesis of Title Compound

The title compound was prepared as described for Example 2, Step 2, using rac-(R)-1-(6-(2-hydroxyphenyl)pyridazin-4-yl)-3-methoxy-N-methyl-N-(piperidin-4-yl)pyrrolidine-3-carboxamide hydrochloride (Example 55, Step 1) and rac-(R)-2-(1-(4-(2,6-dioxopiperidin-3-yl)-3,5-difluorophenyl)piperidin-4-yl)acetaldehyde. After RP-HPLC, this compound was subjected to 2 stages of chiral SFC purification (Chiralpak IH, EtOH+60ACN+0.1% NH₄OH followed by Whelk-O1 (R,R), EtOH+60ACN+0.10% NH₄OH). The peak was collected and concentrated to afford the title compound as a white solid (Peak 2 after second SFC stage, RT=7.69 min, 27.4 mg, 11% yield). Assignment of absolute stereochemistry is arbitrary. LCMS C₄₀H₄₉F₂N₇O₅ requires 745.4, found m/z=746.4 [M+H]⁺.

### Example 83

### RAC-(R)—N-(1-(2-(1-(4-((S)-2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-3-ISOPROPOXY-N-METHYLPYRROLIDINE-3-CARBOXAMIDE

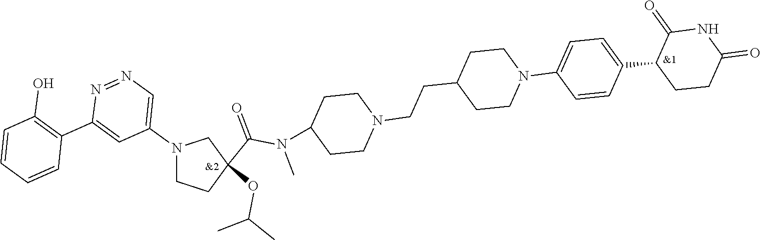 [SMILES: CC(C)O[C@@]1(C(=O)N(C)C2CCN(CCC3CCN(c4ccc([C@H]5CCC(=O)NC5=O)cc4)CC3)CC2)CCN(c2cnnc(-c3ccccc3O)c2)C1]

### Step 1: Synthesis of methyl 2-isopropoxyacrylate

To a stirred solution of methyl 2-methoxyacrylate (13 g, 111 mmol) in ⁱPrOH (150 mL) was added CuCl₂ (903 mg, 6.7 mmol), NaH₂PO₄ (9.40 g, 78 mmol) and Bis(benzonitrile)palladium (II) dichloride (4.29 g, 11.1 mmol) and the reaction mixture was stirred at RT for 48 h. After completion of reaction (monitored by TLC), charcoal (10 g) was added to the reaction mixture and the solution was stirred at RT for another 30 min. The reaction mixture was then filtered through a fritted funnel and washed with diethyl ether (250 mL). The filtrate was diluted with water (200 mL) and the organic layer was separated. The aqueous layer was further extracted with diethyl ether (2×250 mL). The combined organic layers were dried over anhydrous Na₂SO₄ and concentrated under reduced pressure (below 30° C.) to get the desired compound as colorless liquid (6.5 g, 40%). ¹H NMR (400 MHz, CDCl₃-d₆): δ 5.41 (d, J=2.0 Hz, 1H), 4.61 (d, J=2.0 Hz, 1H), 4.32-4.26 (m, 1H), 3.82 (s, 3H), 1.35-1.32 (m, 6H).

### Step 2: Synthesis of methyl 1-benzyl-3-isopropoxypyrrolidine-3-carboxylate

To a stirred solution of methyl 2-isopropoxyacrylate (2.5 g, 17 mmol) and N-benzyl-1-methoxy-N-((trimethylsilyl)methyl)methanamine (16.46 g, 68 mmol) in acetonitrile (30 mL) was added LiF (1.35 g, 52 mmol) at RT and the resulting reaction mixture was stirred at 70° C. for 16 h. After completion of reaction (monitored by TLC and LCMS), reaction mixture was concentrated under reduced pressure to get a crude residue which was purified by flash column chromatography (5% EtOAc in heptane) to get the desired compound as pale yellow sticky gum (1.5 g, 31%).

### Step 3: Synthesis of methyl 1-benzyl-3-isopropoxypyrrolidine-3-carboxylate

To a stirred solution of methyl 1-benzyl-3-isopropoxypyrrolidine-3-carboxylate (750 mg, 2.7 mmol) in EtOH (10 mL) was added 10% Pd/C (375 mg) and HOONH₄ (852 mg, 13 mmol) at RT under inert atmosphere and the resulting mixture was stirred at 70° C. for 2 h. After completion of the reaction (monitored by TLC and LCMS), the reaction mixture was filtered through a celite bed and washed with EtOH (50 mL). The filtrate was concentrated under reduced pressure to get the desired compound as colorless viscous liquid (430 mg, 84%).

### Step 4: Synthesis of methyl 3-isopropoxy-1-(6-(2-(methoxymethoxy)phenyl)pyridazin-4-yl)pyrrolidine-3-carboxylate

To a stirred solution of methyl 3-isopropoxypyrrolidine-3-carboxylate 6 (400 mg, 2.1 mmol) and 5-chloro-3-(2-(methoxymethoxy)phenyl)pyridazine 7 (535 mg, 2.1 mmol) in DMA (10 mL) was added DIPEA (1.14 mL, 6.4 mmol) and the reaction mixture was stirred at 100° C. for 8 h. After completion of reaction (monitored by LCMS), the reaction mixture was diluted with cold water (50 mL) and extracted with EtOAc (2×50 mL). The combined organic layers were dried over anhydrous Na₂SO₄, filtered and concentrated under reduced pressure. The crude residue was purified by flash column chromatography (3% MeOH in DCM) to get desired compound as yellow gummy solid (430 mg, 50%).

### Step 5: Synthesis of 3-isopropoxy-1-(6-(2-(methoxymethoxy)phenyl)pyridazin-4-yl)pyrrolidine-3-carboxylic acid

To a stirred solution of methyl 3-isopropoxy-1-(6-(2-(methoxymethoxy)phenyl)pyridazin-4-yl)pyrrolidine-3-carboxylate (430 mg, 1.07 mmol) in THF (5 mL) was added a solution of LiOH·H₂O (45 mg, 1.07 mmol) in water (2.5 mL) and the resulting solution was stirred at RT for 16 h. After completion of reaction (monitored by TLC), the mixture was concentrated under reduced pressure, diluted with 25% IPA in CHCl₃ (50 mL) and filtered through a fritted funnel. The filtrate was concentrated under reduced pressure to get the desired compound as a grey solid (400 mg, 96%). ¹H NMR (400 MHz, DMSO-d₆): δ 8.51 (d, J=2.8 Hz, 1H), 7.64 (dd, J=1.6 Hz, 7.6 Hz, 1H), 7.41-7.36 (m, 1H), 7.21 (d, J=8.0 Hz, 1H), 7.12-7.08 (m, 1H), 6.79 (d, J=2.8 Hz, 1H), 5.21 (s, 2H), 3.92-3.83 (m, 1H), 3.68 (brs, 1H), 3.41-3.37 (m, 3H), 3.33 (s, 3H), 2.32-2.25 (m, 1H), 1.99-1.93 (m, 1H), 1.07 (d, J=6.0 Hz, 3H) and 0.98 (d, J=6.0 Hz, 3H).

### Step 6: Synthesis of 3-isopropoxy-1-(6-(2-(methoxymethoxy)phenyl)pyridazin-4-yl)pyrrolidine-3-carboxylic acid

To an ice-cold stirred solution of 3-isopropoxy-1-(6-(2-(methoxymethoxy)phenyl)pyridazin-4-yl)pyrrolidine-3-carboxylic acid Lithium salt (200 mg, 0.51 mmol) and tert-butyl 4-(methylamino)-1-piperidinecarboxylate (165 mg, 0.77 mmol) in DMF (3 mL) was added DIPEA (0.45 mL, 2.5 mmol) under inert atmosphere followed by HATU (215 mg, 0.56 mmol) and the reaction mixture was stirred at RT for 2 h. After completion of the reaction (monitored by TLC and LCMS), the reaction mixture was diluted with water (20 mL) and extracted with 5% MeOH in DCM (2×50 mL). The combined organic layers were dried over anhydrous Na₂SO₄, filtered and concentrated under reduced pressure. The crude residue was purified by flash column chromatography (eluent 2% MeOH in DCM) to get the desired compound as a green sticky solid (200 mg, 67%).

### Step 7: Synthesis of 1-(6-(2-hydroxyphenyl)pyridazin-4-yl)-3-isopropoxy-N-methyl-N-(piperidin-4-yl) pyrrolidine-3-carboxamide

To an ice-cold stirred solution of tert-butyl 4-(3-isopropoxy-1-(6-(2-(methoxymethoxy)phenyl)pyridazin-4-yl)-N-methylpyrrolidine-3-carboxamido) piperidine-1-carboxylate (200 mg, 0.34 mmol) in DCM (5 mL) was added TFA (1 mL) under inert atmosphere and the reaction mixture was stirred at RT for 3 h. After completion of reaction (monitored by TLC and LCMS), the reaction mixture was concentrated under reduced pressure. The crude residue was triturated with diethyl ether (10 mL) and Pentane (10 mL) to get the desired compound as an off-white solid (140 mg, 92%).

### Step 8: Synthesis of N-(1-(2-(1-(4-((R)-2,6-dioxopiperidin-3-yl)phenyl)piperidin-4-yl)ethyl)piperidin-4-yl)-1-(6-(2-hydroxyphenyl)pyridazin-4-yl)-3-isopropoxy-N-methylpyrrolidine-3-carboxamide

To an ice-cold stirred solution of 1-(6-(2-hydroxyphenyl)pyridazin-4-yl)-3-isopropoxy-N-methyl-N-(piperidin-4-yl)pyrrolidine-3-carboxamide (140 mg, 0.31 mmol) and (R)-2-(1-(4-(2,6-dioxopiperidin-3-yl)phenyl)piperidin-4-yl)acetaldehyde (100 mg, 0.31 mmol) in MeOH (1.5 mL) and ACN (4.5 mL) was added DIPEA (205 mg, 1.5 mmol) and the reaction mixture was stirred at RT for 30 min. Reaction mixture was then cooled to 0° C., and sodium triacetoxyborohydride (202 mg, 0.95 mmol) was added and the resulting mixture was stirred at RT for another 2 h. After completion of reaction (monitored by TLC & LCMS), the mixture was quenched with water (20 mL) and extracted with 10% MeOH in DCM (2×30 mL). The combined organic layers were dried over anhydrous Na₂SO₄, filtered and concentrated under reduced pressure. The crude product was purified by RP-HPLC purification using Column: Atlantis T3 C-18 (19×250) mm, 5p) Mobile phase: A:—0.1% FA in Water B:—100% ACN and Flow rate: 20 ml/min., to get the title compound as an off white solid (30 mg, 12%). ¹H NMR (400 MHz, DMSO-d₆): δ 14.79 (brs, 1H), 10.76 (s, 1H), 8.66 (s, 1H), 8.10-8.08 (m, 1H), 7.35-7.27 (m, 2H), 7.04-7.01 (m, 2H), 6.94-6.87 (m, 4H), 4.35-4.28 (m, 1H), 3.98-3.87 (m, 2H), 3.83-3.78 (m, 1H), 3.77-3.72 (m, 1H), 3.69-3.63 (m, 2H), 3.57-3.51 (m, 2H), 3.12 (brs, 4H), 2.75 (s, 1H), 2.67-2.58 (m, 4H), 2.44-2.42 (m, 4H), 2.24 (brs, 1H), 2.11-2.07 (m, 1H), 2.03-1.99 (m, 1H), 1.87-1.65 (m, 5H), 1.58 (brs, 2H), 1.46 (brs, 3H), 1.29-1.21 (m, 2H) and 1.12-1.03 (m, 6H).

### Example 84

### (S)—N-(1-((1-(2-((RS)-2,6-DIOXOPIPERIDIN-3-YL)-1-OXO-1,2-DIHYDROISOQUINOLIN-6-YL)PIPERIDIN-4-YL)METHYL)PIPERIDIN-4-YL)-1-(6-(2-HYDROXYPHENYL)PYRIDAZIN-4-YL)-3-METHOXY-N-METHYLPYRROLIDINE-3-CARBOXAMIDE

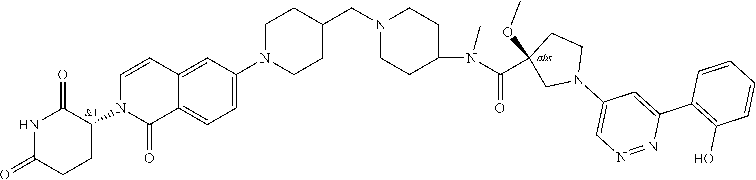 [SMILES: CO[C@@]1(C(=O)N(C)C2CCN(CC3CCN(c4ccc5c(=O)n([C@@H]6CCC(=O)NC6=O)ccc5c4)CC3)CC2)CCN(c2cnnc(-c3ccccc3O)c2)C1]

### Step 1: Synthesis of (S)-1-(6-(2-hydroxyphenyl)pyridazin-4-yl)-3-methoxy-N-methyl-N-(piperidin-4-yl)pyrrolidine-3-carboxamide hydrochloride

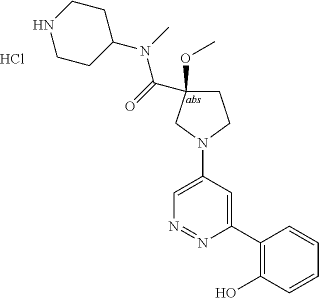 [SMILES: Cl ; CO[C@@]1(C(=O)N(C)C2CCNCC2)CCN(c2cnnc(-c3ccccc3O)c2)C1]

The title compound was prepared as described for Example 1, Step 1, using (S)-3-methoxy-1-(6-(2-(methoxymethoxy)phenyl)pyridazin-4-yl)pyrrolidine-3-carboxylic acid (Intermediate 25-1) and tert-butyl 4-(methylamino)piperidine-1-carboxylate to afford the title compound as a yellow oil. LCMS C₂₂H₂₉N₅O₃ requires 411.2, found m/z=412.2 [M+H]⁺.

### Step 2: Synthesis of the Title Compound

The title compound was prepared as described for Example 2, Step 2, using (S)-1-(6-(2-hydroxyphenyl)pyridazin-4-yl)-3-methoxy-N-methyl-N-(piperidin-4-yl)pyrrolidine-3-carboxamide hydrochloride (Example 84, Step 1) and rac-(R)-1-(2-(2,6-dioxopiperidin-3-yl)-1-oxo-1,2-dihydroisoquinolin-6-yl)piperidine-4-carbaldehyde (WO2021168197 A1) to afford the title compound (4.6 mgs, 49% yield) as a white solid. LCMS C₄₂H₅₀N₈O₆ requires: 762.4, found: m/z=763.4 [M+H]⁺.

### Example 85

### (S)—N-(1-(2-(1-(4-((RS)-2,6-DIOXOPIPERIDIN-3-YL)PHENYL)-4-HYDROXYPIPERIDIN-4-YL)ETHYL)PIPERIDIN-4-YL)-1-(6-(2-HYDROXYPHENYL)PYRIDAZIN-4-YL)-3-METHOXY-N-METHYLPYRROLIDINE-3-CARBOXAMIDE

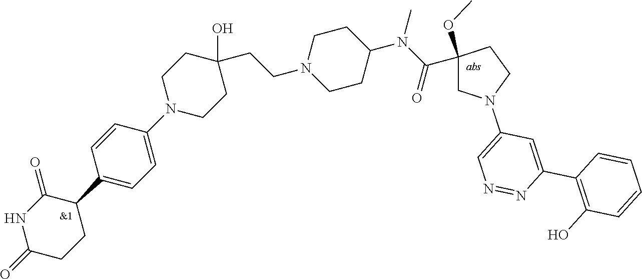 [SMILES: CO[C@@]1(C(=O)N(C)C2CCN(CCC3(O)CCN(c4ccc([C@H]5CCC(=O)NC5=O)cc4)CC3)CC2)CCN(c2cnnc(-c3ccccc3O)c2)C1]

### Step 1: Synthesis of Title Compound

The title compound was prepared as described for Example 1, Step 2, using (S)-1-(6-(2-hydroxyphenyl)pyridazin-4-yl)-3-methoxy-N-methyl-N-(piperidin-4-yl)pyrrolidine-3-carboxamide hydrochloride (Example 84, Step 1) and rac-(R)-2-(1-(4-(2,6-dioxopiperidin-3-yl)phenyl)-4-hydroxypiperidin-4-yl)acetaldehyde to afford the title compound (8.5 mg, 57% yield) as a white solid. LCMS C₄₀H₅₁N₇O₆ requires: 725.4, found: m/z=726.3 [M+H]⁺.

### Example 86

### (S)—N-(1-((1-(4-((R)-2,6-DIOXOPIPERIDIN-3-YL)-3-FLUOROPHENYL)PIPERIDIN-4-YL)METHYL)PIPERIDIN-4-YL)-1-(6-(2-HYDROXYPHENYL)PYRIDAZIN-4-YL)-3-METHOXY-N-METHYLPYRROLIDINE-3-CARBOXAMIDE

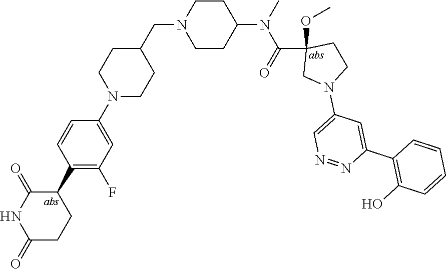 [SMILES: CO[C@@]1(C(=O)N(C)C2CCN(CC3CCN(c4ccc([C@H]5CCC(=O)NC5=O)c(F)c4)CC3)CC2)CCN(c2cnnc(-c3ccccc3O)c2)C1]

### Step 1: Synthesis of the Title Compound

The title compound was prepared as described for Example 1, Step 2 using (S)-1-(6-(2-hydroxyphenyl)pyridazin-4-yl)-3-methoxy-N-methyl-N-(piperidin-4-yl)pyrrolidine-3-carboxamide hydrochloride (Example 84, Step 1) and (R)-1-(4-(2,6-dioxopiperidin-3-yl)-3-fluorophenyl)piperidine-4-carbaldehyde to afford the title compounds (3.8 mgs, 42% yield) as a white solid. LCMS C₃₉H₄₈FN₇O₅ requires: 713.4, found: m/z=714.4 [M+H]⁺.

### Example 87

### (S)—N-(1-(2-(1-(5-((RS)-2,6-DIOXOPIPERIDIN-3-YL)PYRIDIN-2-YL)PIPERIDIN-4-YL)ETHYL)PIPERIDIN-4-YL)-1-(6-(2-HYDROXYPHENYL)PYRIDAZIN-4-YL)-3-METHOXY-N-METHYLPYRROLIDINE-3-CARBOXAMIDE

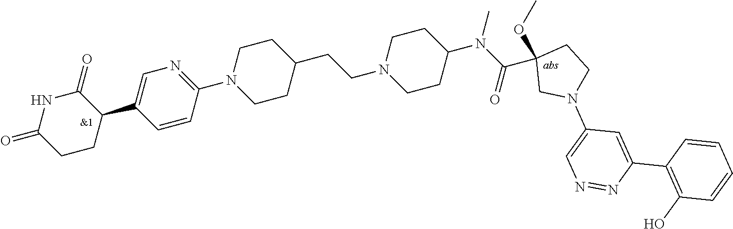 [SMILES: CO[C@@]1(C(=O)N(C)C2CCN(CCC3CCN(c4ccc([C@H]5CCC(=O)NC5=O)cn4)CC3)CC2)CCN(c2cnnc(-c3ccccc3O)c2)C1]

### Step 1: Synthesis of the Title Compound

The title compound was prepared as described for Example 1, Step 2, using (S)-1-(6-(2-hydroxyphenyl)pyridazin-4-yl)-3-methoxy-N-methyl-N-(piperidin-4-yl)pyrrolidine-3-carboxamide hydrochloride (Example 84, Step 1) and rac-(R)-2-(1-(5-(2,6-dioxopiperidin-3-yl)pyridin-2-yl)piperidin-4-yl)acetaldehyde to afford the title compound (6.9 mgs, 57% yield) as a white solid. LCMS: C₃₉H₅₀N₈O₅ requires: 710.4, found: m/z=711.3 [M+H]⁺.

### Example 88

### (S)—N-(1-(7-(4-((RS)-2,6-DIOXOPIPERIDIN-3-YL)PHENYL)-7-AZASPIRO[3.5]NONAN-2-YL)PIPERIDIN-4-YL)-1-(6-(2-HYDROXYPHENYL)PYRIDAZIN-4-YL)-3-METHOXY-N-METHYLPYRROLIDINE-3-CARBOXAMIDE

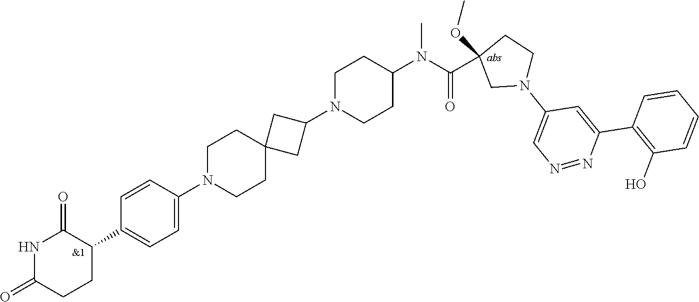 [SMILES: CO[C@@]1(C(=O)N(C)C2CCN(C3CC4(CCN(c5ccc([C@@H]6CCC(=O)NC6=O)cc5)CC4)C3)CC2)CCN(c2cnnc(-c3ccccc3O)c2)C1]

### Step 1: Synthesis of the Title Compound

The title compound was prepared as described for the synthesis of Example 1, Step 2, using (S)-1-(6-(2-hydroxyphenyl)pyridazin-4-yl)-3-methoxy-N-methyl-N-(piperidin-4-yl)pyrrolidine-3-carboxamide hydrochloride (Example 84, Step 1) and rac-(R)-3-(4-(2-oxo-7-azaspiro[3.5]nonan-7-yl)phenyl)piperidine-2,6-dione to afford the title compound (5.5 mgs, 52% yield) as a white solid. LCMS: C₄₁H₅₁N₇O₅ requires: 721.4, found: m/z=722.3 [M+H]⁺.

### Example 89

### (R)—N-(2,2-DIFLUOROETHYL)-N-(1-(2-(1-(4-(2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-4-METHOXYPIPERIDINE-4-CARBOXAMIDE

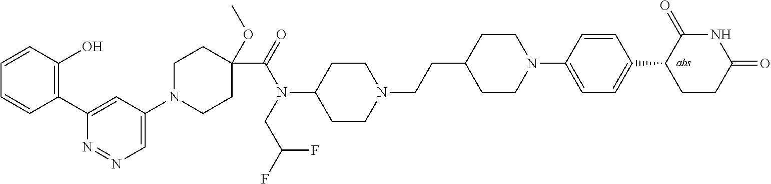 [SMILES: COC1(C(=O)N(CC(F)F)C2CCN(CCC3CCN(c4ccc([C@H]5CCC(=O)NC5=O)cc4)CC3)CC2)CCN(c2cnnc(-c3ccccc3O)c2)CC1]

### Step 1: Synthesis of N-(2,2-difluoroethyl)-1-(6-(2-hydroxyphenyl)pyridazin-4-yl)-4-methoxy-N-(piperidin-4-yl)piperidine-4-carboxamide

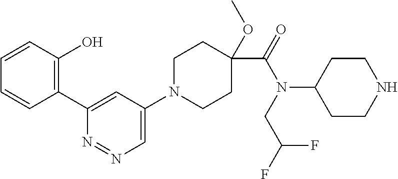 [SMILES: COC1(C(=O)N(CC(F)F)C2CCNCC2)CCN(c2cnnc(-c3ccccc3O)c2)CC1]

Lithio 4-methoxy-1-{6-[2-(methoxymethoxy)phenyl]pyridazin-4-yl}piperidine-4-carboxylate (300.00 mg, 0.79 mmol) was dissolved in dry acetonitrile (12.00 mL). The flask was cooled to 0° C. and a drop of DMF was added. Then, oxalyl chloride (271.28 μL, 3.16 mmol) was added dropwise as a solution in 2 mL of acetonitrile. The reaction mixture was stirred at room temperature until complete by LCMS. Then, the reaction mixture was concentrated to approximately half of the initial volume. The acid chloride concentrated solution was then filtered through a syringe filter directly into an ice cold solution of tert-butyl 4-[(2,2-difluoroethyl)amino]piperidine-1-carboxylate (313.55 mg, 1.19 mmol) and N,N-diisopropylethylamine (414.38 μL, 306.64 mg, 2.37 mmol) in acetonitrile (0.25 mL). The reaction was slowly warmed to room temperature and then warmed to 50° C. and stirred until complete by LCMS. Then, the reaction was quenched with water, washed with a saturated solution of K₂CO₃, extracted with DCM, dried, filtered and concentrated under reduced pressure. The crude product was purified on silica gel with a gradient of EtOAc/Hex 0-100%, then 0-10% EtOAc/MeOH to afford the pure product as a yellow oil (195 mg, 40%). The pure material was dissolved in 2.00 mL of DCM and 4M HCl in Dioxane (1.00 mL) was added in one portion. The reaction was stirred at room temperature until complete by LCMS and then concentrated in vacuo to yield the desired product as a yellow foam. LCMS: C₂₄H₃₁F₂N₅O₃ requires 475.2, found: m/z=476.2 [M+H]⁺.

### Step 2: Synthesis of the Title Compound

The title compound was prepared as described for Example 1, Step 2, using N-(2,2-difluoroethyl)-1-(6-(2-hydroxyphenyl)pyridazin-4-yl)-4-methoxy-N-(piperidin-4-yl)piperidine-4-carboxamide and (R)-2-(1-(4-(2,6-dioxopiperidin-3-yl)phenyl)piperidin-4-yl)acetaldehyde to afford the title compound (22 mg, 41%). ¹H NMR (700 MHz, DMSO) δ 10.78 (s, 1H), 8.94 (d, J=2.8 Hz, 1H), 8.12-8.10 (m, 1H), 7.56 (d, J=2.9 Hz, 1H), 7.33 (t, J=7.6 Hz, 1H), 7.02 (d, J=8.4 Hz, 2H), 6.92 (d, J=7.8 Hz, 2H), 6.88 (d, J=8.5 Hz, 2H), 6.14 (tt, J=57.3, 4.5 Hz, 1H), 4.67-4.60 (m, 1H), 3.94 (d, J=13.8 Hz, 2H), 3.71 (dd, J=11.4, 5.2 Hz, 1H), 3.64 (d, J=12.1 Hz, 2H), 3.59 (td, J=13.7, 4.0 Hz, 2H), 3.47-3.32 (m, 3H), 3.23 (s, 3H), 2.99 (d, J=11.1 Hz, 2H), 2.67-2.58 (m, 3H), 2.45 (dt, J=17.1, 4.4 Hz, 1H), 2.36 (t, J=7.1 Hz, 2H), 2.12 (dd, J=12.0, 3.9 Hz, 1H), 2.06-1.92 (m, 7H), 1.76 (dd, J=24.3, 11.5 Hz, 4H), 1.59 (d, J=11.9 Hz, 2H), 1.40 (t, J=6.6 Hz, 3H), 1.28-1.19 (m, 2H). LCMS: C₄₂H₅₃F₂N₇O₅ requires 773.4, found: m/z=774.3 [M+H]⁺.

### Example 90

### (R)—N-(1-(2-(1-(4-(2,6-DIOXOPIPERIDIN-3-YL)PHENYL)PIPERIDIN-4-YL)ETHYL)PIPERIDIN-4-YL)-N-ETHYL-1-(6-(5-FLUORO-2-HYDROXYPHENYL)PYRIDAZIN-4-YL)-4-METHOXYPIPERIDINE-4-CARBOXAMIDE

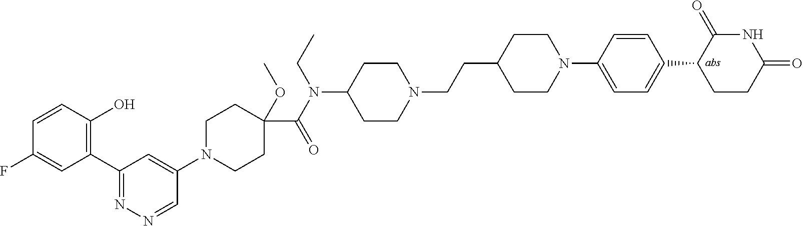 [SMILES: CCN(C(=O)C1(OC)CCN(c2cnnc(-c3cc(F)ccc3O)c2)CC1)C1CCN(CCC2CCN(c3ccc([C@H]4CCC(=O)NC4=O)cc3)CC2)CC1]

### Step 1: Synthesis of N-ethyl-1-(6-(5-fluoro-2-hydroxyphenyl)pyridazin-4-yl)-4-methoxy-N-(piperidin-4-yl)piperidine-4-carboxamide

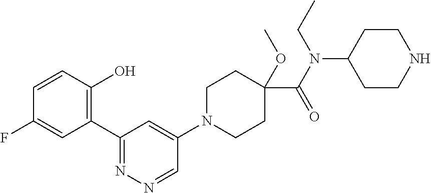 [SMILES: CCN(C(=O)C1(OC)CCN(c2cnnc(-c3cc(F)ccc3O)c2)CC1)C1CCNCC1]

To a solution of lithium 1-(6-(5-fluoro-2-(methoxymethoxy)phenyl)pyridazin-4-yl)-4-methoxypiperidine-4-carboxylate (1.00 g, 2.52 mmol) and tert-butyl 4-(ethylamino)piperidine-1-carboxylate (0.90 g, 3.95 mmol) in DCM (15.00 mL) was added N,N-diisopropylethylamine (1.84 mL, 1.36 g, 10.5 mmol). Then bis(2-oxo-1,3-oxazolidin-3-yl)phosphinoyl chloride (2.01 g, 7.90 mmol) was added and the reaction mixture was stirred at 40° C. overnight. Once complete, the reaction was quenched with water, extracted with DCM (3×50 mL), dried with Na₂SO₄, filtered and concentrated under reduced pressure. The crude material was purified via chromatography (MeOH:DCM, gradient) to yield the desired product as an orange foam. The obtained material was dissolved in 10 mL methanol and 4M HCl in Dioxane (10.0 mL) was added in one portion. The reaction was stirred at RT for 1 hour and then concentrated in vacuo to yield the desired product (1.22 g, 98%) as a white solid. LCMS: C₂₄H₃₂N₅O₃F requires: 457.2, found: m/z=457.3 [M+H]⁺.

### Step 2: Synthesis of the Title Compound

N-ethyl-1-[6-(5-fluoro-2-hydroxyphenyl)pyridazin-4-yl]-4-methoxy-N-(piperidin-4-yl)piperidine-4-carboxamide hydrochloride (300.00 mg, 0.6073 mmol) was dissolved in MeCN (4.50 mL) and 2-(1-{4-[(3R)-2,6-dioxopiperidin-3-yl]phenyl}piperidin-4-yl)acetaldehyde (210.01 mg, 0.6680 mmol) was added in one portion. The flask was agitated and then MeOH (1.30 mL) was slowly added until all the solids have dissolved. Sodium triacetoxyborohydride (386.12 mg, 1.8218 mmol) was then added in one portion and the reaction was stirred at RT for 30 mins. After 30 mins, the reaction mixture was dissolved in DCM (10 mL) and water (20 mL) was added. The organic phase was separated and the aqueous layer was further extracted with DCM (2×10 mL). The combined organic phases were dried over sodium sulfate, filtered and concentrated in vacuo. The residue was purified by RP-FC to yield the title compound (275 mg, 59.5%) as a white solid. LCMS: C₄₂H₅₄FN₇O₅ requires: 755.4, found: m/z=756.3 [M+H]⁺.

### Example 91

### (R)—N-(1-(2-(1-(4-(2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-N-ISOPROPOXY-4-METHOXYPIPERIDINE-4-CARBOXAMIDE

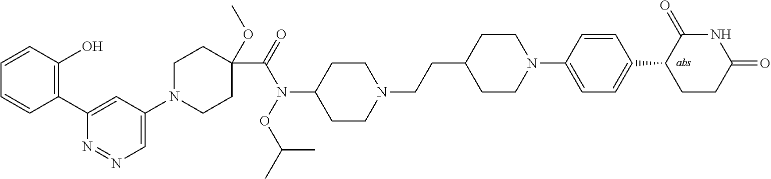 [SMILES: COC1(C(=O)N(OC(C)C)C2CCN(CCC3CCN(c4ccc([C@H]5CCC(=O)NC5=O)cc4)CC3)CC2)CCN(c2cnnc(-c3ccccc3O)c2)CC1]

### Step 1: Synthesis of 1-(6-(2-hydroxyphenyl)pyridazin-4-yl)-N-isopropoxy-4-methoxy-N-(piperidin-4-yl)piperidine-4-carboxamide

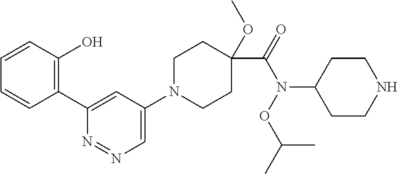 [SMILES: COC1(C(=O)N(OC(C)C)C2CCNCC2)CCN(c2cnnc(-c3ccccc3O)c2)CC1]

The title compound was prepared as described for Example 89, Step 1, using tert-butyl 4-(isopropoxyamino)piperidine-1-carboxylate to afford the desired product as a white solid. LCMS: C₂₅H₃₅N₅O₄ requires 469.3, found: m/z=470.2 [M+H]⁺.

### Step 2: Synthesis of the Title Compound

The title compound was prepared as described for Example 1, Step 2, using 1-(6-(2-hydroxyphenyl)pyridazin-4-yl)-N-isopropoxy-4-methoxy-N-(piperidin-4-yl)piperidine-4-carboxamide and (R)-2-(1-(4-(2,6-dioxopiperidin-3-yl)phenyl)piperidin-4-yl)acetaldehyde to afford the title compound (2.1 mg, 6%). LCMS: C₄₃H₅₇N₇O₆ requires 767.4, found: m/z=768.4 [M+H]⁺.

### Example 92

### RAC-(1R,5S,8S)—N-(1-(2-(1-(4-((R)-2,6-DIOXOPIPERIDIN-3-YL)-3-FLUOROPHENYL)PIPERIDIN-4-YL)ETHYL)PIPERIDIN-4-YL)-3-(6-(2-HYDROXYPHENYL)PYRIDAZIN-4-YL)-N-(THIAZOL-5-YLMETHYL)-3-AZABICYCLO[3.2.1]OCTANE-8-CARBOXAMIDE

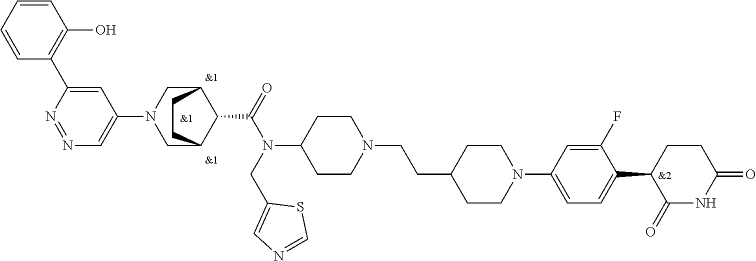 [SMILES: O=C1CC[C@H](c2ccc(N3CCC(CCN4CCC(N(Cc5cncs5)C(=O)[C@H]5[C@@H]6CC[C@H]5CN(c5cnnc(-c7ccccc7O)c5)C6)CC4)CC3)cc2F)C(=O)N1]

### Step 1: Synthesis of (1R,5S,8s)-3-(6-(2-hydroxyphenyl)pyridazin-4-yl)-N-(piperidin-4-yl)-N-(thiazol-5-ylmethyl)-3-azabicyclo[3.2.1]octane-8-carboxamide

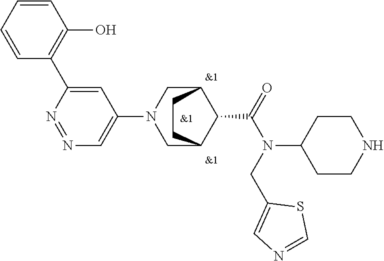 [SMILES: O=C([C@H]1[C@@H]2CC[C@H]1CN(c1cnnc(-c3ccccc3O)c1)C2)N(Cc1cncs1)C1CCNCC1]

To a solution of (1R,5S,8S)-3-{6-[2-(methoxymethoxy)phenyl]pyridazin-4-yl}-3-azabicyclo[3.2.1]octane-8-carboxylic acid (45.00 mg, 0.12 mmol) and tert-butyl 4-[(1,3-thiazol-5-ylmethyl)amino]piperidine-1-carboxylate (43.48 mg, 0.15 mmol) in DCM (1.00 mL) was added N,N-diisopropylethylamine (85.10 μL, 0.06 g, 0.49 mmol). Then, bis(2-oxo-1,3-oxazolidin-3-yl)phosphinoyl chloride (93.03 mg, 0.37 mmol) was added and the reaction mixture was stirred at 40° C. until complete by LCMS. Then, the reaction mixture was filtered and the solvent removed under reduced pressure. The crude product was purified on silica gel with a gradient of EtOAc/Hex 0-100%, then 0-10% EtOAc/MeOH to afford the pure product as a yellow oil (77 mg, 99%). The pure material was dissolved in 1.5 mL of DCM and 4M HCl in Dioxane (1.00 mL) was added in one portion. The reaction was stirred at room temperature until complete by LCMS and then concentrated in vacuo to yield the desired product as a yellow foam. LCMS: C₂₇H₃₂N₆O₂S requires 504.2, found: m/z=505.2 [M+H]⁺.

### Step 2: Synthesis of the Title Compound

The title compound was prepared as described for Example 1, Step 2, using (1R,5S,8s)-3-(6-(2-hydroxyphenyl)pyridazin-4-yl)-N-(piperidin-4-yl)-N-(thiazol-5-ylmethyl)-3-azabicyclo[3.2.1]octane-8-carboxamide and rac-(R)-2-(1-(4-(2,6-dioxopiperidin-3-yl)-3-fluorophenyl)piperidin-4-yl)acetaldehyde to afford the title compound (9.2 mg, 49%). LCMS: C₄₅H₅₃FN₈O₄ requires 820.4, found: m/z=821.4 [M+H]⁺.

### Example 93

### RAC-(R)—N-(1-(2-(1-(4-(2,6-DIOXOPIPERIDIN-3-YL)-3-FLUOROPHENYL)PIPERIDIN-4-YL)ETHYL)PIPERIDIN-4-YL)-1-(6-(2-HYDROXYPHENYL)PYRIDAZIN-4-YL)-4-METHOXY-N-(2,2,2-TRIFLUOROETHYL)PIPERIDINE-4-CARBOXAMIDE

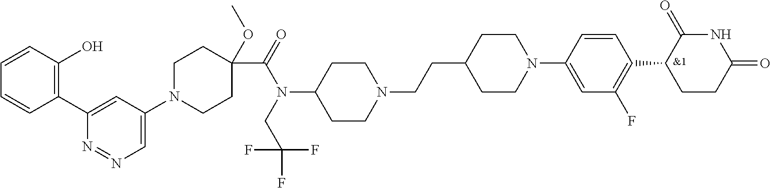 [SMILES: COC1(C(=O)N(CC(F)(F)F)C2CCN(CCC3CCN(c4ccc([C@H]5CCC(=O)NC5=O)c(F)c4)CC3)CC2)CCN(c2cnnc(-c3ccccc3O)c2)CC1]

### Step 2: Synthesis of the Title Compound

The title compound was prepared as described for Example 1, Step 2, using 1-(6-(2-hydroxyphenyl)pyridazin-4-yl)-4-methoxy-N-(piperidin-4-yl)-N-(2,2,2-trifluoroethyl)piperidine-4-carboxamide and rac-(R)-2-(1-(4-(2,6-dioxopiperidin-3-yl)-3-fluorophenyl)piperidin-4-yl)acetaldehyde to afford the title compound (12.4 mg, 76%). LCMS: C₄₂H₅₁F₄N₇O₅ requires 809.4, found: m/z=810.3 [M+H]⁺.

### Example 94

### RAC-(1R,5S,8S)—N-(1-(2-(1-(4-((R)-2,6-DIOXOPIPERIDIN-3-YL)-3,5-DIFLUOROPHENYL)PIPERIDIN-4-YL)ETHYL)PIPERIDIN-4-YL)-N-((1-HYDROXYCYCLOPROPYL)METHYL)-3-(6-(2-HYDROXYPHENYL)PYRIDAZIN-4-YL)-3-AZABICYCLO[3.2.1]OCTANE-8-CARBOXAMIDE

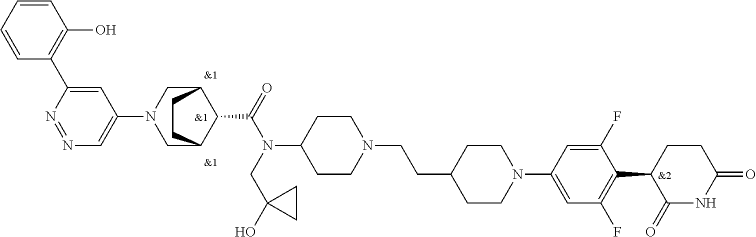 [SMILES: O=C1CC[C@H](c2c(F)cc(N3CCC(CCN4CCC(N(CC5(O)CC5)C(=O)[C@H]5[C@@H]6CC[C@H]5CN(c5cnnc(-c7ccccc7O)c5)C6)CC4)CC3)cc2F)C(=O)N1]

### Step 1: Synthesis of tert-butyl 4-(((1-methoxycyclopropyl)methyl)amino)piperidine-1-carboxylate

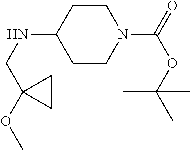 [SMILES: COC1(CNC2CCN(C(=O)OC(C)(C)C)CC2)CC1]

To a vial containing tert-butyl 4-oxopiperidine-1-carboxylate (500.00 mg, 2.51 mmol) dissolved in 1,2-dichloroethane (2.00 mL) was added 1-(1-methoxycyclopropyl)methanamine hydrochloride (414.38 mg, 3.01 mmol), N,N-diisopropylethylamine (876.60 μL, 0.65 g, 5.02 mmol) and the reaction was allowed to stir at room temperature. After 30 minutes, sodium triacetoxyborohydride (797.77 mg, 3.76 mmol) was added in one portion and the reaction was allowed to stir at room temperature overnight. The reaction mixture was dissolved with CH₂Cl₂, quenched with a saturated solution of NaHCO₃, extracted with CH₂Cl₂, dried over Na₂SO₄, filtered and the solvent was removed under reduced pressure. The crude product was purified on silica gel with a gradient of MeOH/CH₂Cl₂ 0-20% to afford the title compound (331 mg, 46%) as a colorless oil. LCMS: C₁₅H₂₈N₂O₃ requires 284.2, found: m/z=285.2 [M+H]⁺.

### Step 2: Synthesis of (1R,5S,8S)—N-((1-hydroxycyclopropyl)methyl)-3-(6-(2-hydroxyphenyl)pyridazin-4-yl)-N-(piperidin-4-yl)-3-azabicyclo[3.2.1]octane-8-carboxamide

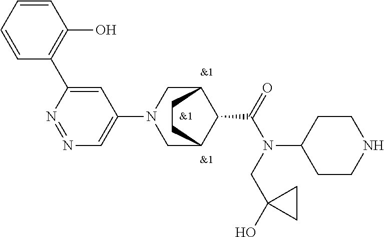 [SMILES: O=C([C@H]1[C@@H]2CC[C@H]1CN(c1cnnc(-c3ccccc3O)c1)C2)N(CC1(O)CC1)C1CCNCC1]

The title compound was prepare as described or Example 6, Step 1, using tert-butyl 4-(((1-methoxycyclopropyl)methyl)amino)piperidine-1-carboxylate to afford the desired product as a beige foam. TFA in CH₂Cl₂ was used for the cleavage of the BOC protecting group. LCMS: C₂₇H₃₅N₅O₃ requires 477.3, found: m/z=478.2 [M+H]⁺.

### Step 3: Synthesis of the Title Compound

The title compound was prepared as described for Example 1, Step 2, using (1R,5S,8S)—N-((1-hydroxycyclopropyl)methyl)-3-(6-(2-hydroxyphenyl)pyridazin-4-yl)-N-(piperidin-4-yl)-3-azabicyclo[3.2.1]octane-8-carboxamide and rac-(R)-2-(1-(4-(2,6-dioxopiperidin-3-yl)-3,5-difluorophenyl)piperidin-4-yl)acetaldehyde to afford the title compound (10.3 mg, 62%). LCMS: C₄₅H₅₇N₇O₅ requires 775.4, found: m/z=776.4 [M+H]⁺.

### Example 95

### N-(1-(2-(1-(4-((R)-2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-4-METHOXY-N—((R)-1-METHOXYPROPAN-2-YL)PIPERIDINE-4-CARBOXAMIDE

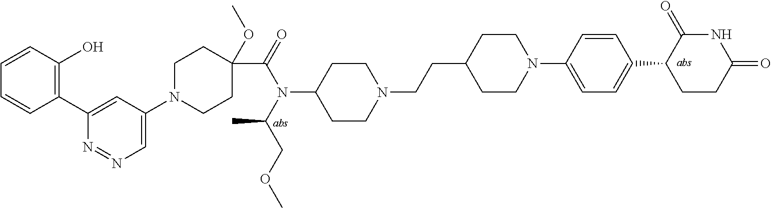 [SMILES: COC[C@@H](C)N(C(=O)C1(OC)CCN(c2cnnc(-c3ccccc3O)c2)CC1)C1CCN(CCC2CCN(c3ccc([C@H]4CCC(=O)NC4=O)cc3)CC2)CC1]

### Step 1: Synthesis of tert-butyl (R)-4-((1-methoxypropan-2-yl)amino)piperidine-1-carboxylate

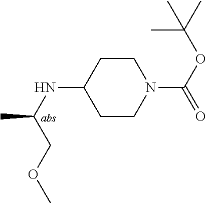 [SMILES: COC[C@@H](C)NC1CCN(C(=O)OC(C)(C)C)CC1]

The title compound was prepared as described for Example 94, Step 1, using (2R)-1-methoxypropan-2-amine hydrochloride (367.71 μL, 268.43 mg, 3.01 mmol) to afford the desired product as a colorless oil. LCMS: C₁₄H₂₈N₂O₃ requires 272.2, found: m/z=273.2 [M+H]⁺.

### Step 2: Synthesis of (R)-1-(6-(2-hydroxyphenyl)pyridazin-4-yl)-4-methoxy-N-(1-methoxypropan-2-yl)-N-(piperidin-4-yl)piperidine-4-carboxamide

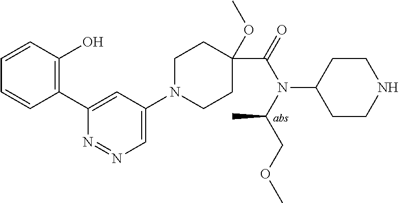 [SMILES: COC[C@@H](C)N(C(=O)C1(OC)CCN(c2cnnc(-c3ccccc3O)c2)CC1)C1CCNCC1]

The title compound was prepared as described for Example 89, Step 1, using tert-butyl (R)-4-((1-methoxypropan-2-yl)amino)piperidine-1-carboxylate to afford the desired product as a brown oil. LCMS: C₂₆H₃₇N₅O₄ requires 483.3, found: m/z=484.2 [M+H]⁺.

### Step 3: Synthesis of the Title Compound

The title compound was prepared as described for Example 1, Step 2, using (R)-1-(6-(2-hydroxyphenyl)pyridazin-4-yl)-4-methoxy-N-(1-methoxypropan-2-yl)-N-(piperidin-4-yl)piperidine-4-carboxamide and (R)-2-(1-(4-(2,6-dioxopiperidin-3-yl)phenyl)piperidin-4-yl)acetaldehyde to afford the title compound (20.9 mg, 27%). LCMS: C₄₄H₅₉N₇O₆ requires 781.5, found: m/z=782.4 [M+H]⁺.

### Example 96

### (R)—N-(2-CYANOETHYL)-N-(1-(2-(1-(4-(2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-4-METHOXYPIPERIDINE-4-CARBOXAMIDE

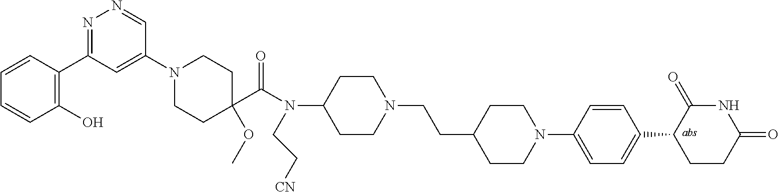 [SMILES: COC1(C(=O)N(CCC#N)C2CCN(CCC3CCN(c4ccc([C@H]5CCC(=O)NC5=O)cc4)CC3)CC2)CCN(c2cnnc(-c3ccccc3O)c2)CC1]

### Step 1: Synthesis of N-(2-cyanoethyl)-1-(6-(2-hydroxyphenyl)pyridazin-4-yl)-4-methoxy-N-(piperidin-4-yl)piperidine-4-carboxamide

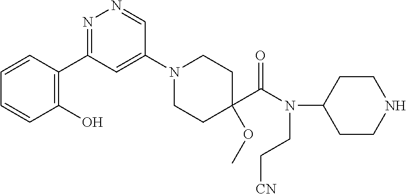 [SMILES: COC1(C(=O)N(CCC#N)C2CCNCC2)CCN(c2cnnc(-c3ccccc3O)c2)CC1]

To a solution of lithio 4-methoxy-1-{6-[2-(methoxymethoxy)phenyl]pyridazin-4-yl}piperidine-4-carboxylate (367.00 mg, 0.9675 mmol) and tert-butyl 4-[(2-cyanoethyl)amino]piperidine-1-carboxylate (367.66 mg, 1.4512 mmol) in DCM (5.00 mL) was added N,N-diisopropylethylamine (0.68 mL, 500.17 mg, 3.8699 mmol). Then bis(2-oxo-1,3-oxazolidin-3-yl)phosphinoyl chloride (738.84 mg, 2.9024 mmol) was added and the reaction mixture was stirred at 40° C. overnight. Once complete, the reaction was quenched with water (5 mL), extracted with DCM (3×10 mL), dried over Na₂SO₄, filtered and concentrated in vacuo. The crude material was purified via chromatography (MeOH:DCM gradient) to yield the desired product. The obtained material was dissolved in 10 mL methanol and 4M HCl in Dioxane (10.0 mL) was added in one portion. The reaction was stirred at RT for 1 hour and then concentrated in vacuo to yield the desired product (1.22 g) as a white solid. LCMS: C₃₂H₄₄N₆O₆ requires 464.2, found m/z=465.2 [M+H]⁺.

### Step 2: Synthesis of the Title Compound

N-(2-cyanoethyl)-1-[6-(2-hydroxyphenyl)pyridazin-4-yl]-4-methoxy-N-(piperidin-4-yl)piperidine-4-carboxamide hydrochloride (30.00 mg, 0.0607 mmol) was dissolved in MeCN (0.45 mL) and 2-(1-{4-[(3R)-2,6-dioxopiperidin-3-yl]phenyl}piperidin-4-yl)acetaldehyde (21.00 mg, 0.0668 mmol) was added in one portion. The flask was agitated and then MeOH (0.13 mL) was slowly added until all the solids have dissolved. Sodium triacetoxyborohydride (38.61 mg, 0.1822 mmol) was then added in one portion and the reaction was stirred at RT for 30 mins. After 30 mins, the reaction was checked by LCMS and the reaction mixture was dissolved in DCM and water was added. The reaction was extracted 3× with DCM and concentrated in vacuo. The residue was then purified by RP—FC to yield the title compound (9.4 mg, 17.6%) as a white solid. LCMS: C₄₃H₅₄N₈O₅ requires: 762.4, found: m/z=763.4 [M+H]⁺.

### Example 97

### (R)—N—(CYANOMETHYL)-N-(1-(2-(1-(4-(2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-4-METHOXYPIPERIDINE-4-CARBOXAMIDE

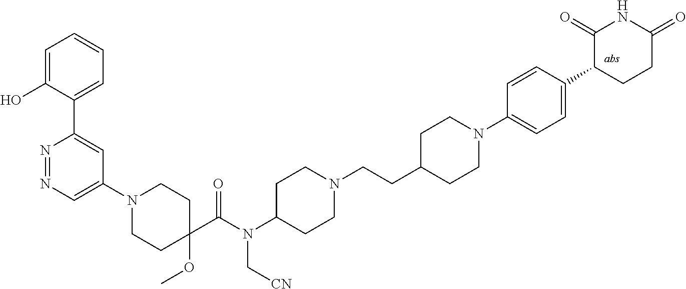 [SMILES: COC1(C(=O)N(CC#N)C2CCN(CCC3CCN(c4ccc([C@H]5CCC(=O)NC5=O)cc4)CC3)CC2)CCN(c2cnnc(-c3ccccc3O)c2)CC1]

### Step 1: Synthesis of the Title Compound

N-(cyanomethyl)-1-[6-(2-hydroxyphenyl)pyridazin-4-yl]-4-methoxy-N-(piperidin-4-yl)piperidine-4-carboxamide hydrochloride (20.00 mg, 0.0411 mmol) was dissolved in MeCN (0.45 mL) and 2-(1-{4-[(3R)-2,6-dioxopiperidin-3-yl]phenyl}piperidin-4-yl)acetaldehyde (14.20 mg, 0.0452 mmol) was added in one portion. The flask was agitated and then MeOH (0.13 mL) was slowly added until all the solids have dissolved. Sodium triacetoxyborohydride (26.11 mg, 0.1232 mmol) was then added in one portion and the reaction was stirred at RT for 30 mins. After 30 mins, the reaction was checked by LCMS and the reaction mixture was dissolved in DCM and water was added. The reaction was extracted 3× with DCM and concentrated in vacuo. The residue was then purified via RP—FC to afford the title compound (4.1 mg, 10.27%) as the formate salt. LCMS: C₄₂H₅₂N₈O₅ requires: 748.4, found: m/z=749.3 [M+H]⁺.

### Example 98

### (1RS,5SR,8SR)—N-(1-(2-(1-(4-((RS)-2,6-DIOXOPIPERIDIN-3-YL)-3,5-DIFLUOROPHENYL)PIPERIDIN-4-YL)ETHYL)PIPERIDIN-4-YL)-3-(6-(2-HYDROXYPHENYL)PYRIDAZIN-4-YL)-N—((R)-2-METHOXYPROPYL)-3-AZABICYCLO[3.2.1]OCTANE-8-CARBOXAMIDE

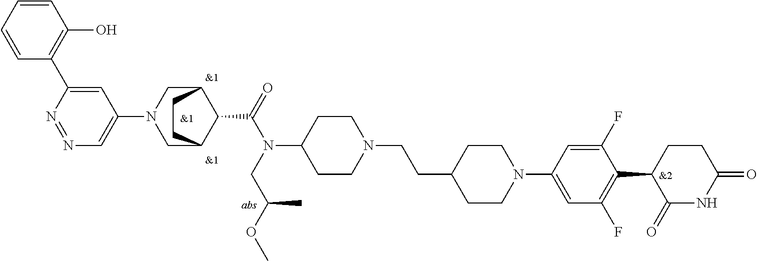 [SMILES: CO[C@H](C)CN(C(=O)[C@H]1[C@@H]2CC[C@H]1CN(c1cnnc(-c3ccccc3O)c1)C2)C1CCN(CCC2CCN(c3cc(F)c([C@H]4CCC(=O)NC4=O)c(F)c3)CC2)CC1]

### Step 1: Synthesis of tert-butyl (R)-4-((2-methoxypropyl)amino)piperidine-1-carboxylate

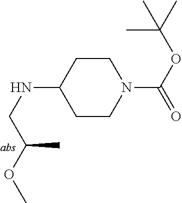 [SMILES: CO[C@H](C)CNC1CCN(C(=O)OC(C)(C)C)CC1]

The title compound was prepared as described for Example 94, Step 1, using tert-butyl 4-oxopiperidine-1-carboxylate (500.01 mg, 2.51 mmol) to afford the desired product as a colorless oil. LCMS: C₁₄H₂₈N₂O₃ requires 272.2, found: m/z=273.2 [M+H]⁺.

### Step 2: Synthesis of (1RS,5SR,8SR)-3-(6-(2-hydroxyphenyl)pyridazin-4-yl)-N—((R)-2-methoxypropyl)-N-(piperidin-4-yl)-3-azabicyclo[3.2.1]octane-8-carboxamide

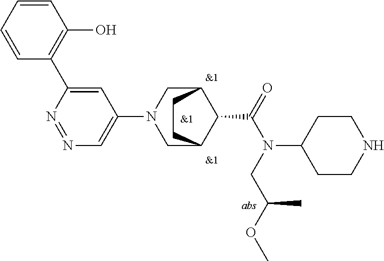 [SMILES: CO[C@H](C)CN(C(=O)[C@H]1[C@@H]2CC[C@H]1CN(c1cnnc(-c3ccccc3O)c1)C2)C1CCNCC1]

The title compound was prepared as described for Example 6, Step 1, using tert-butyl (R)-4-((2-methoxypropyl)amino)piperidine-1-carboxylate to afford the desired product as a yellow foam. TFA in CH₂Cl₂ was used for the cleavage of the BOC protecting group. LCMS: C₂₇H₃₇N₅O₃ requires 479.3, found: m/z=480.2 [M+H]⁺.

### Step 3: Synthesis of the Title Compound

The title compound was prepared as described for Example 1, Step 2, using (1RS,5SR,8SR)-3-(6-(2-hydroxyphenyl)pyridazin-4-yl)-N—((R)-2-methoxypropyl)-N-(piperidin-4-yl)-3-azabicyclo[3.2.1]octane-8-carboxamide and rac-(R)-2-(1-(4-(2,6-dioxopiperidin-3-yl)-3,5-difluorophenyl)piperidin-4-yl)acetaldehyde to afford the title compound (8.3 mg, 47%). LCMS: C₄₅H₅₇F₂N₇O₅ requires 813.4, found: m/z=814.5 [M+H]⁺.

### Example 99

### RAC-(R)—N-(1-(2-(1-(1-(2,6-DIOXOPIPERIDIN-3-YL)-3-METHYL-2-oxo-2,3-DIHYDRO-1H-BENZO[D]IMIDAZOL-4-YL)PIPERIDIN-4-YL)ETHYL)PIPERIDIN-4-YL)-N-ETHYL-1-(6-(2-HYDROXYPHENYL)PYRIDAZIN-4-YL)-4-METHOXYPIPERIDINE-4-CARBOXAMIDE

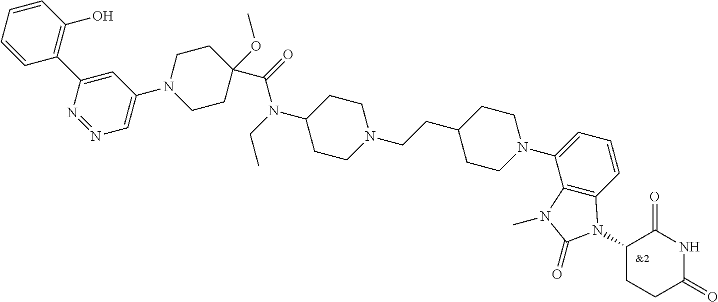 [SMILES: CCN(C(=O)C1(OC)CCN(c2cnnc(-c3ccccc3O)c2)CC1)C1CCN(CCC2CCN(c3cccc4c3n(C)c(=O)n4[C@H]3CCC(=O)NC3=O)CC2)CC1]

### Step 1: Synthesis of the Title Compound

The title compound was prepared as described for Example 1, Step 2, using N-ethyl-1-(6-(2-hydroxyphenyl)pyridazin-4-yl)-4-methoxy-N-(piperidin-4-yl)piperidine-4-carboxamide and rac-(R)-2-(1-(1-(2,6-dioxopiperidin-3-yl)-3-methyl-2-oxo-2,3-dihydro-1H-benzo[d]imidazol-4-yl)piperidin-4-yl)acetaldehyde to afford the title compound (10.1 mg, 28%). LCMS: C₄₄H₅₇N₉O₆ requires 807.4, found: m/z=808.4 [M+H]⁺.

### Example 100

### (1RS&,5SR&,8RS&)—N-{1-[2-(1-{1-[(3RS)-2,6-DIOXOPIPERIDIN-3-YL]-3-METHYL-2-OXO-2,3-DIHYDRO-1H-1,3-BENZODIAZOL-4-YL}PIPERIDIN-4-YL)ETHYL]PIPERIDIN-4-YL}-3-[6-(2-HYDROXYPHENYL)PYRIDAZIN-4-YL]-N—(PROPAN-2-YLOXY)-3-AZABICYCLO[3.2.1]OCTANE-8-CARBOXAMIDE

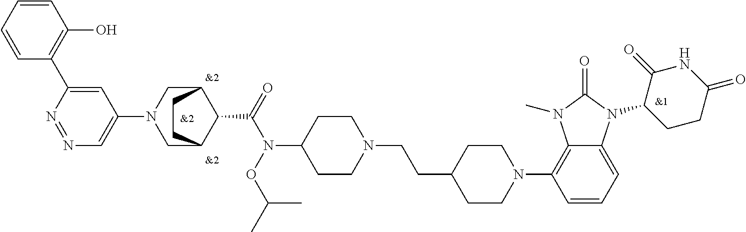 [SMILES: CC(C)ON(C(=O)[C@H]1[C@@H]2CC[C@H]1CN(c1cnnc(-c3ccccc3O)c1)C2)C1CCN(CCC2CCN(c3cccc4c3n(C)c(=O)n4[C@H]3CCC(=O)NC3=O)CC2)CC1]

### Step 1: tert-butyl 4-[N-isopropoxy(1R,5S,8S)-3-{6-[2-(methoxymethoxy)phenyl]pyridazin-4-yl}-3-azabicyclo[3.2.1]octane-8-amido]piperidine-1-carboxylate

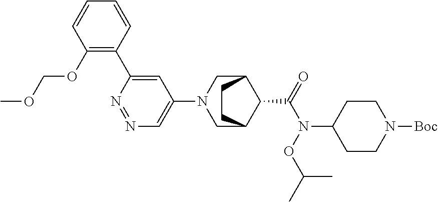 [SMILES: COCOc1ccccc1-c1cc(N2C[C@H]3CC[C@@H](C2)[C@H]3C(=O)N(OC(C)C)C2CCN(C(=O)OC(C)(C)C)CC2)cnn1]

To a solution of (1R,5S,8S)-3-{6-[2-(methoxymethoxy)phenyl]pyridazin-4-yl}-3-azabicyclo[3.2.1]octane-8-carboxylic acid (45.01 mg, 0.1218 mmol) in DMA (1.00 mL) was added tert-butyl 4-(isopropoxyamino)piperidine-1-carboxylate (37.77 mg, 0.1462 mmol) and N,N-diisopropylethylamine (106.11 μL, 0.08 g, 0.6092 mmol). [(dimethylamino)({[1,2,3]triazolo[4,5-b]pyridin-3-yloxy})methylidene]dimethylazanium; hexafluoro-lambda5-phosphanuide (55.59 mg, 0.1462 mmol) was added and the reaction was stirred at rt overnight. Added more HATU (55 mg) and continued stirring at 45° C. overnight. Purified directly by RP-HPLC to yield tert-butyl 4-[N-isopropoxy(1R,5S,8S)-3-{6-[2-(methoxymethoxy)phenyl]pyridazin-4-yl}-3-azabicyclo[3.2.1]octane-8-amido]piperidine-1-carboxylate (0.0304 g, 34.47%). LCMS: C₃₃H₄₇N₅O₆ requires: 609.4, found: m/z=610.2 [M+H]⁺.

### Step 2: (1R,5S,8S)-3-[6-(2-hydroxyphenyl)pyridazin-4-yl]-N-isopropoxy-N-(piperidin-4-yl)-3-azabicyclo[3.2.1]octane-8-carboxamide

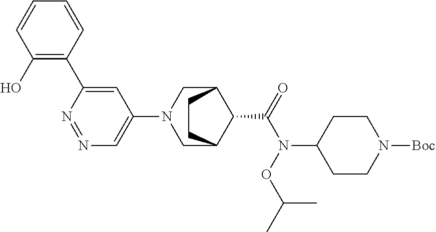 [SMILES: CC(C)ON(C(=O)[C@H]1[C@@H]2CC[C@H]1CN(c1cnnc(-c3ccccc3O)c1)C2)C1CCN(C(=O)OC(C)(C)C)CC1]

(1R,5S,8S)-3-[6-(2-hydroxyphenyl)pyridazin-4-yl]-N-isopropoxy-N-(piperidin-4-yl)-3-azabicyclo[3.2.1]octane-8-carboxamide was prepared as described for Example 107 step 2. LCMS: C₂₆H₃₅N₅O₃ requires: 465.3, found: m/z=466.2 [M+H]⁺.

### Step: 3 Synthesis of the Title Compound

The title compound was synthesized as described for Example 153 step 5, using (1R,5S,8S)-3-[6-(2-hydroxyphenyl)pyridazin-4-yl]-N-isopropoxy-N-(piperidin-4-yl)-3-azabicyclo[3.2.1]octane-8-carboxamide hydrochloride and rac-2-(1-{1-[(3R)-2,6-dioxopiperidin-3-yl]-3-methyl-2-oxo-1,3-benzodiazol-4-yl}piperidin-4-yl)acetaldehyde to yield (1RS&,5SR&,8RS&)—N-{1-[2-(1-{1-[(3RS)-2,6-dioxopiperidin-3-yl]-3-methyl-2-oxo-1,3-benzodiazol-4-yl}piperidin-4-yl)ethyl]piperidin-4-yl}-3-[6-(2-hydroxyphenyl)pyridazin-4-yl]-N-isopropoxy-3-azabicyclo[3.2.1]octane-8-carboxamide (0.0064 g, 55.76%). LCMS: C₄₆H₅₉N₉O₆ requires: 833.5, found: m/z=834.3 [M+H]⁺.

### Example 101

### (R)—N-(1-(2-(1-(4-(2,6-DIOXOPIPERIDIN-3-YL)-3,5-DIFLUOROPHENYL)PIPERIDIN-4-YL)ETHYL)PIPERIDIN-4-YL)-N-ETHYL-1-(6-(5-FLUORO-2-HYDROXYPHENYL)PYRIDAZIN-4-YL)-4-METHOXYPIPERIDINE-4-CARBOXAMIDE

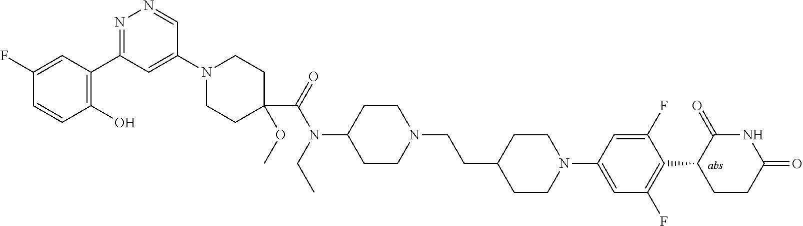 [SMILES: CCN(C(=O)C1(OC)CCN(c2cnnc(-c3cc(F)ccc3O)c2)CC1)C1CCN(CCC2CCN(c3cc(F)c([C@H]4CCC(=O)NC4=O)c(F)c3)CC2)CC1]

### Step 1: Synthesis of the Title Compound

N-ethyl-1-[6-(5-fluoro-2-hydroxyphenyl)pyridazin-4-yl]-4-methoxy-N-(piperidin-4-yl)piperidine-4-carboxamide hydrochloride (13.00 mg, 0.0263 mmol) was dissolved in DMA (1.00 mL) and 2-(1-{4-[(3R)-2,6-dioxopiperidin-3-yl]-3,5-difluorophenyl}piperidin-4-yl)acetaldehyde (8.75 mg, 0.0263 mmol) was added in one portion. N,N-diisopropylethylamine (0.02 mL, 17.01 mg, 0.1316 mmol) was then added followed by sodium triacetoxyborohydride (16.73 mg, 0.0789 mmol). The resulting reaction was stirred at RT for 1-2 hrs and monitored via LCMS. Once complete, the reaction was concentrated in purified by RP—FC to afford the title compound (11.7 mg, 53.1%) as a formate salt. LCMS: C₄₂H₅₂F₃N₇O₅ requires: 791.4, found: m/z=792.3 [M+H]⁺.

### Example 102

### (R)—N-(1-(2-(1-(4-(2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-4-METHOXY-N—(OXAZOL-4-YLMETHYL)PIPERIDINE-4-CARBOXAMIDE

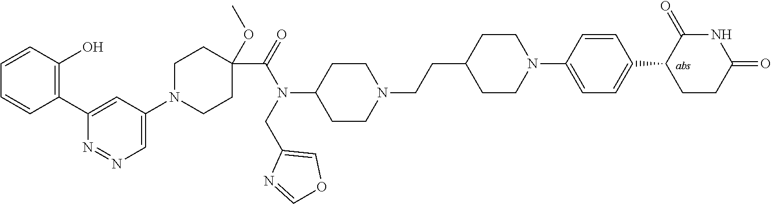 [SMILES: COC1(C(=O)N(Cc2cocn2)C2CCN(CCC3CCN(c4ccc([C@H]5CCC(=O)NC5=O)cc4)CC3)CC2)CCN(c2cnnc(-c3ccccc3O)c2)CC1]

### Step 1: Synthesis of 1-(6-(2-hydroxyphenyl)pyridazin-4-yl)-4-methoxy-N-(oxazol-4-ylmethyl)-N-(piperidin-4-yl)piperidine-4-carboxamide

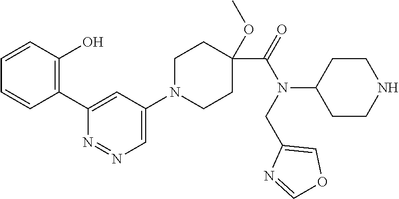 [SMILES: COC1(C(=O)N(Cc2cocn2)C2CCNCC2)CCN(c2cnnc(-c3ccccc3O)c2)CC1]

The title compound was prepared as described for Example 92, Step 1, using tert-butyl 4-((oxazol-4-ylmethyl)amino)piperidine-1-carboxylate to afford the desired product as a yellow foam. LCMS: C₂₆H₃₂N₆O₄ requires 492.3, found: m/z=493.2 [M+H]⁺.

### Step 2: Synthesis of the Title Compound

The title compound was prepared as described for Example 1, Step 2, using 1-(6-(2-hydroxyphenyl)pyridazin-4-yl)-4-methoxy-N-(oxazol-4-ylmethyl)-N-(piperidin-4-yl)piperidine-4-carboxamide and (R)-2-(1-(4-(2,6-dioxopiperidin-3-yl)phenyl)piperidin-4-yl)acetaldehyde to afford the title compound (35.8 mg, 81%). LCMS: C₄₄H₅₄N₈O₆ requires 790.4, found: m/z=791.3 [M+H]⁺.

### Example 103

### (R)—N-(1-(2-(1-(4-(2,6-DIOXOPIPERIDIN-3-YL)-3-FLUOROPHENYL)PIPERIDIN-4-YL)ETHYL)PIPERIDIN-4-YL)-1-(6-(5-FLUORO-2-HYDROXYPHENYL)PYRIDAZIN-4-YL)-4-METHOXY-N-PROPYLPIPERIDINE-4-CARBOXAMIDE

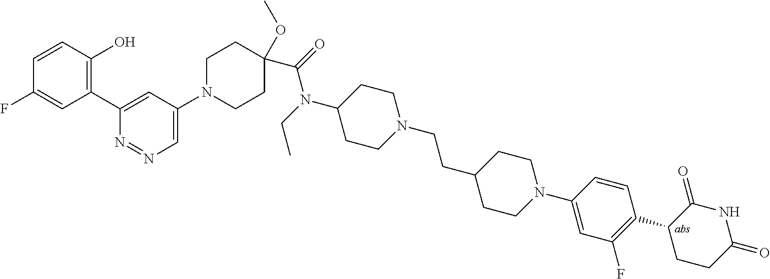 [SMILES: CCN(C(=O)C1(OC)CCN(c2cnnc(-c3cc(F)ccc3O)c2)CC1)C1CCN(CCC2CCN(c3ccc([C@H]4CCC(=O)NC4=O)c(F)c3)CC2)CC1]

### Step 1: Synthesis of 1-(6-(5-fluoro-2-hydroxyphenyl)pyridazin-4-yl)-4-methoxy-N-(piperidin-4-yl)-N-propylpiperidine-4-carboxamide

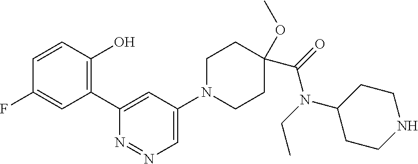 [SMILES: CCN(C(=O)C1(OC)CCN(c2cnnc(-c3cc(F)ccc3O)c2)CC1)C1CCNCC1]

To a solution of lithio 1-{6-[5-fluoro-2-(methoxymethoxy)phenyl]pyridazin-4-yl}-4-methoxypiperidine-4-carboxylate (300.00 mg, 0.7550 mmol) and tert-butyl 4-(propylamino)piperidine-1-carboxylate (274.49 mg, 1.1326 mmol) in DCM (4.00 mL) was added N,N-diisopropylethylamine (390.35 mg, 3.0202 mmol). Then bis(2-oxo-1,3-oxazolidin-3-yl)phosphinoyl chloride (576.61 mg, 2.2651 mmol) was added and the reaction mixture was stirred at 40 C. Once complete, the reaction was quenched with water (5 mL), extracted with DCM (3×10 mL), dried over Na₂SO₄, filtered and concentrated in vacuo. The crude material was purified via chromatography (MeOH:DCM gradient) to yield the desired product. The obtained material was dissolved in 1 mL methanol and 4M HCl in Dioxane (1.0 mL) was added in one portion. The reaction was stirred at RT for 1 hour and then concentrated in vacuo to yield the desired product (292 mg, 62.1%) as an orange solid. LCMS: C₂₅H₃₄FN₅O₃ requires 471.2, found m/z=472.5 [M+H]⁺.

### Step 2: Synthesis of the Title Compound

1-[6-(5-fluoro-2-hydroxyphenyl)pyridazin-4-yl]-4-methoxy-N-(piperidin-4-yl)-N-propylpiperidine-4-carboxamide hydrochloride (11.00 mg, 0.0217 mmol) was dissolved in DMA (1.00 mL) and 2-(1-{4-[(3R)-2,6-dioxopiperidin-3-yl]-3-fluorophenyl}piperidin-4-yl)acetaldehyde (7.20 mg, 0.0217 mmol) was added in one portion. N,N-diisopropylethylamine (0.02 mL, 13.99 mg, 0.1083 mmol) was then added followed by sodium triacetoxyborohydride (13.77 mg, 0.0650 mmol). The resulting reaction was stirred at RT for 1-2 hrs and monitored via LCMS. Once complete, the reaction was concentrated in vacuo and purified by RP—FC to yield the title compound (13.1 mg, 71.1%) as a formate salt. LCMS: C₄₃H₅₅F₂N₇O₅ requires: 787.4, found: m/z=788.3 [M+H]⁺.

### Example 104

### (1RS&,5SR&,8RS&)—N-(2,2-DIFLUOROETHYL)-N-{1-[2-(1-{1-[(3RS)-2,6-DIOXOPIPERIDIN-3-YL]-3-METHYL-2-OXO-2,3-DIHYDRO-1H-1,3-BENZODIAZOL-4-YL}PIPERIDIN-4-YL)ETHYL]PIPERIDIN-4-YL}-3-[6-(2-HYDROXYPHENYL)PYRIDAZIN-4-YL]-3-AZABICYCLO[3.2.1]OCTANE-8-CARBOXAMIDE

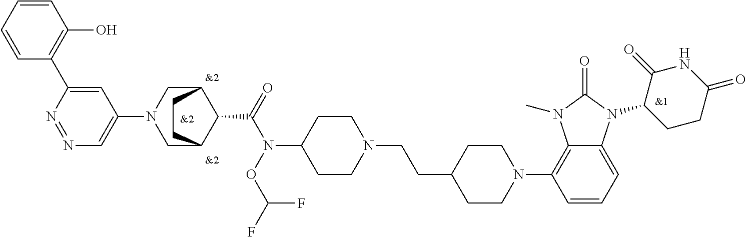 [SMILES: Cn1c(=O)n([C@H]2CCC(=O)NC2=O)c2cccc(N3CCC(CCN4CCC(N(OC(F)F)C(=O)[C@H]5[C@@H]6CC[C@H]5CN(c5cnnc(-c7ccccc7O)c5)C6)CC4)CC3)c21]

### Step 1: tert-butyl 4-[N-(2,2-difluoroethyl)(1R,5S,8S)-3-{6-[2-(methoxymethoxy)phenyl]pyridazin-4-yl}-3-azabicyclo[3.2.1]octane-8-amido]piperidine-1-carboxylate

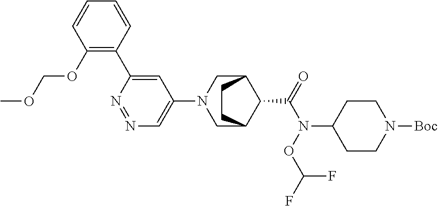 [SMILES: COCOc1ccccc1-c1cc(N2C[C@H]3CC[C@@H](C2)[C@H]3C(=O)N(OC(F)F)C2CCN(C(=O)OC(C)(C)C)CC2)cnn1]

To a solution of (1R,5S,8S)-3-{6-[2-(methoxymethoxy)phenyl]pyridazin-4-yl}-3-azabicyclo[3.2.1]octane-8-carboxylic acid (45.01 mg, 0.1218 mmol) in DMA (1.00 mL) was added tert-butyl 4-[(2,2-difluoroethyl)amino]piperidine-1-carboxylate (48.31 mg, 0.1828 mmol) and N,N-diisopropylethylamine (63.67 μL, 0.05 g, 0.3655 mmol). [(dimethylamino)({[1,2,3]triazolo[4,5-b]pyridin-3-yloxy})methylidene]dimethylazanium; hexafluoro-lambda5-phosphanuide (55.59 mg, 0.1462 mmol) was added. Stirred at rt for 2 h, then added more N,N-diisopropylethylamine (63.67 μL, 0.05 g, 0.3655 mmol) and HATU (55 mg) and stirred overnight. Added more HATU (55 mg) and continued stirring at 40° C. overnight, then added more HATU (50 mg) and then more amine (48 mg) and stirred at 40° C. Purified directly by RP-HPLC to yield tert-butyl 4-[N-(2,2-difluoroethyl)(1R,5S,8S)-3-{6-[2-(methoxymethoxy)phenyl]pyridazin-4-yl}-3-azabicyclo[3.2.1]octane-8-amido]piperidine-1-carboxylate (0.0386 g, 43.41%). LCMS: C₃₂H₄₃F₂N₅O₅ requires: 615.3, found: m/z=616.2 [M+H]⁺.

### Step 2: (1R,5S,8S)—N-(2,2-difluoroethyl)-3-[6-(2-hydroxyphenyl)pyridazin-4-yl]-N-(piperidin-4-yl)-3-azabicyclo[3.2.1]octane-8-carboxamide

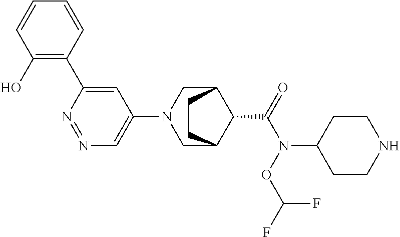 [SMILES: O=C([C@H]1[C@@H]2CC[C@H]1CN(c1cnnc(-c3ccccc3O)c1)C2)N(OC(F)F)C1CCNCC1]

(1R,5S,8S)—N-(2,2-difluoroethyl)-3-[6-(2-hydroxyphenyl)pyridazin-4-yl]-N-(piperidin-4-yl)-3-azabicyclo[3.2.1]octane-8-carboxamide was prepared as described for Example 107 step 2. LCMS: C₂₅H₃₁F₂N₅O₂ requires: 471.2, found: m/z=472.2 [M+H]⁺.

### Step: 3 Synthesis of the Title Compound

The title compound was synthesized as described for Example 153 step 5, using (1R,5S,8S)—N-(2,2-difluoroethyl)-3-[6-(2-hydroxyphenyl)pyridazin-4-yl]-N-(piperidin-4-yl)-3-azabicyclo[3.2.1]octane-8-carboxamide hydrochloride and rac-2-(1-{1-[(3R)-2,6-dioxopiperidin-3-yl]-3-methyl-2-oxo-1,3-benzodiazol-4-yl}piperidin-4-yl)acetaldehyde to yield (1RS&,5SR&,8RS&)—N-(2,2-difluoroethyl)-N-{1-[2-(1-{1-[(3RS)-2,6-dioxopiperidin-3-yl]-3-methyl-2-oxo-1,3-benzodiazol-4-yl}piperidin-4-yl)ethyl]piperidin-4-yl}-3-[6-(2-hydroxyphenyl)pyridazin-4-yl]-3-azabicyclo[3.2.1]octane-8-carboxamide (0.0074 g, 52.90%). LCMS: C₄₅H₅₅F₂N₉O₅ requires: 839.4, found: m/z=840.3 [M+H]⁺.

### Example 105

### (R)—N-(1-(2-(1-(4-(2,6-DIOXOPIPERIDIN-3-YL)PHENYL)PIPERIDIN-4-YL)ETHYL)PIPERIDIN-4-YL)-1-(6-(5-FLUORO-2-HYDROXYPHENYL)PYRIDAZIN-4-YL)-4-METHOXY-N-PROPYLPIPERIDINE-4-CARBOXAMIDE

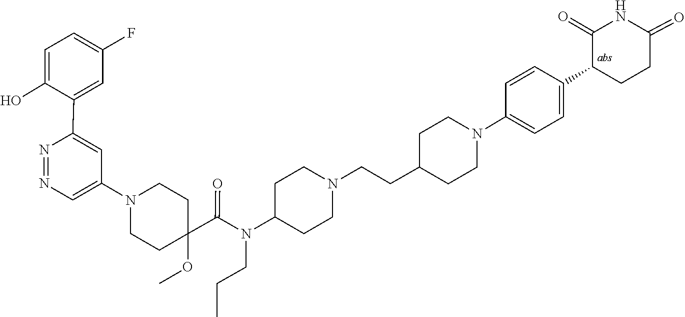 [SMILES: CCCN(C(=O)C1(OC)CCN(c2cnnc(-c3cc(F)ccc3O)c2)CC1)C1CCN(CCC2CCN(c3ccc([C@H]4CCC(=O)NC4=O)cc3)CC2)CC1]

### Step 1: Synthesis of the Title Compound

1-[6-(5-fluoro-2-hydroxyphenyl)pyridazin-4-yl]-4-methoxy-N-(piperidin-4-yl)-N-propylpiperidine-4-carboxamide hydrochloride (30.00 mg, 0.0607 mmol) was dissolved in MeCN (0.45 mL) and 2-(1-{4-[(3R)-2,6-dioxopiperidin-3-yl]phenyl}piperidin-4-yl)acetaldehyde (21.00 mg, 0.0668 mmol) was added in one portion. The flask was agitated and then MeOH (0.13 mL) was slowly added until all the solids have dissolved. Sodium triacetoxyborohydride (38.61 mg, 0.1822 mmol) was then added in one portion and the reaction was stirred at RT for 30 mins. After 30 mins, the reaction was checked by LCMS and the reaction mixture was dissolved in DCM and water was added. The reaction was extracted 3× with DCM and concentrated in vacuo. The residue was then purified by RP—FC to yield the title compound (29.2 mg, 57.57%) as a formate salt. LCMS: C₄₃H₅₆FN₇O₅ requires: 769.4, found: m/z=770.3 [M+H]⁺.

### Example 106

### (1RS,5SR,8RS)—N-(2,2-DIFLUOROETHYL)-N-{1-[2-(1-{4-[(3R)-2,6-DIOXOPIPERIDIN-3-YL]PHENYL}PIPERIDIN-4-YL)ETHYL]PIPERIDIN-4-YL}-3-[6-(2-HYDROXYPHENYL)PYRIDAZIN-4-YL]-3-AZABICYCLO[3.2.1]OCTANE-8-CARBOXAMIDE

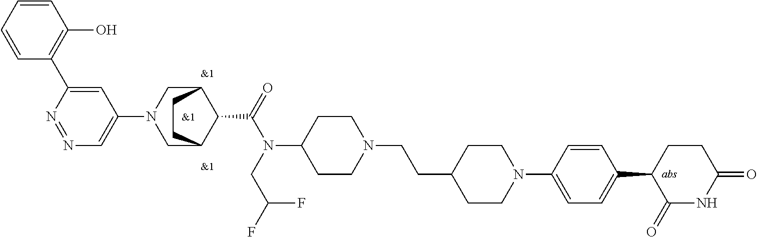 [SMILES: O=C1CC[C@H](c2ccc(N3CCC(CCN4CCC(N(CC(F)F)C(=O)[C@H]5[C@@H]6CC[C@H]5CN(c5cnnc(-c7ccccc7O)c5)C6)CC4)CC3)cc2)C(=O)N1]

Combined (1R,5S,8S)—N-(2,2-difluoroethyl)-3-[6-(2-hydroxyphenyl)pyridazin-4-yl]-N-(piperidin-4-yl)-3-azabicyclo[3.2.1]octane-8-carboxamide hydrochloride (7.90 mg, 0.0156 mmol) and 2-(1-{4-[(3R)-2,6-dioxopiperidin-3-yl]phenyl}piperidin-4-yl)acetaldehyde (5.13 mg, 0.0163 mmol) in DMA (0.40 mL) and then added sodium triacetoxyborohydride (5.93 mg, 0.0280 mmol) and stirred for 1h. Purified by RP-HPLC to yield (1RS,5SR,8RS)—N-(2,2-difluoroethyl)-N-{1-[2-(1-{4-[(3R)-2,6-dioxopiperidin-3-yl]phenyl}piperidin-4-yl)ethyl]piperidin-4-yl}-3-[6-(2-hydroxyphenyl)pyridazin-4-yl]-3-azabicyclo[3.2.1]octane-8-carboxamide (0.0065 g, 50.97%). LCMS: C₄₃H₅₃F₂N₇O₄ requires: 769.4, found: m/z=770.3 [M+H]⁺.

### Example 107

### 4-(DIFLUOROMETHOXY)—N-{1-[2-(1-{4-[(3R)-2,6-DIOXOPIPERIDIN-3-YL]PHENYL}PIPERIDIN-4-YL)ETHYL]PIPERIDIN-4-YL}-N-ETHYL-1-[6-(2-HYDROXYPHENYL)PYRIDAZIN-4-YL]PIPERIDINE-4-CARBOXAMIDE

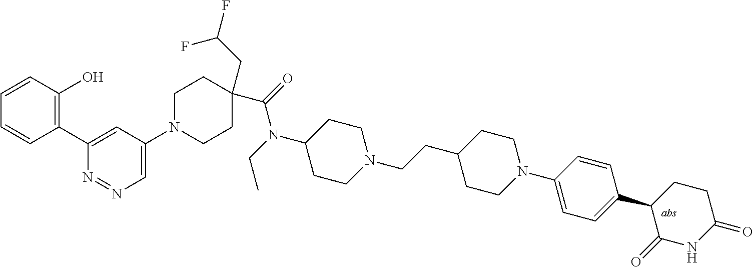 [SMILES: CCN(C(=O)C1(CC(F)F)CCN(c2cnnc(-c3ccccc3O)c2)CC1)C1CCN(CCC2CCN(c3ccc([C@H]4CCC(=O)NC4=O)cc3)CC2)CC1]

### Step 1: tert-butyl 4-[N-ethyl-4-(difluoromethoxy)-1-{6-[2-(methoxymethoxy)phenyl]pyridazin-4-yl}piperidine-4-amido]piperidine-1-carboxylate

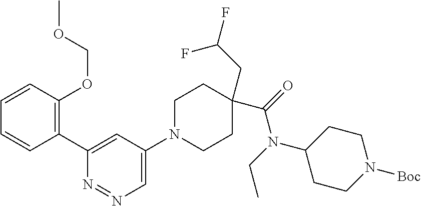 [SMILES: CCN(C(=O)C1(CC(F)F)CCN(c2cnnc(-c3ccccc3OCOC)c2)CC1)C1CCN(C(=O)OC(C)(C)C)CC1]

To a solution of 4-(difluoromethoxy)-1-{6-[2-)(methoxymethoxy)phenyl]pyridazin-4-yl}piperidine-4-carboxylic acid (38.00 mg, 0.0928 mmol) in DMA (0.50 mL) was added tert-butyl 4-(ethylamino)piperidine-1-carboxylate (42.39 mg, 0.1856 mmol) and N,N-diisopropylethylamine (56.59 μL, 0.04 g, 0.3249 mmol). Then, bis(2-oxo-1,3-oxazolidin-3-yl)phosphinoyl chloride (47.26 mg, 0.1856 mmol) was added and the reaction was stirred at rt for 3d. More amine (1 eq, 21 μL) and BOP—Cl (1 eq, 24 mg) were added and the reaction was stirred overnight, then it was purified directly by RP-HPLC (0.1% TFA in water/ACN) to yield tert-butyl 4-[N-ethyl-4-(difluoromethoxy)-1-{6-[2-(methoxymethoxy)phenyl]pyridazin-4-yl}piperidine-4-amido]piperidine-1-carboxylate (0.028 g, 48.68%). LCMS: C₃₁H₄₃F₂N₅O₆ requires: 619.3, found: m/z=620.3 [M+H]⁺.

### Step 2: 4-(difluoromethoxy)-N-ethyl-1-[6-(2-hydroxyphenyl)pyridazin-4-yl]-N-(piperidin-4-yl)piperidine-4-carboxamide

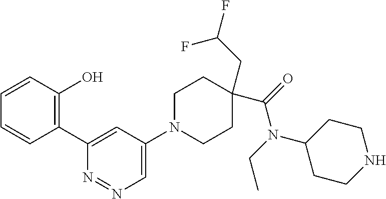 [SMILES: CCN(C(=O)C1(CC(F)F)CCN(c2cnnc(-c3ccccc3O)c2)CC1)C1CCNCC1]

Tert-butyl 4-[N-ethyl-4-(difluoromethoxy)-1-{6-[2-(methoxymethoxy)phenyl]pyridazin-4-yl}piperidine-4-amido]piperidine-1-carboxylate (28.00 mg, 0.0452 mmol) was dissolved in 1 M HCl in dioxane (2.00 mL) and methanol (0.40 mL). After 1h, the reaction was concentrated under reduced pressure and the residue was lyophilized from 1:1 ACN/water to yield tert-butyl 4-[N-ethyl-4-(difluoromethoxy)-1-{6-[2-(methoxymethoxy)phenyl]pyridazin-4-yl}piperidine-4-amido]piperidine-1-carboxylate (28.00 mg, 0.0452 mmol). LCMS: C₂₄H₃₁F₂N₅O₃ requires: 475.2, found: m/z=476.2 [M+H]⁺.

### Step 3: Synthesis of the Title Compound

Combined 4-(difluoromethoxy)-N-ethyl-1-[6-(2-hydroxyphenyl)pyridazin-4-yl]-N-(piperidin-4-yl)piperidine-4-carboxamide hydrochloride (12.00 mg, 0.0234 mmol) and 2-(1-{4-[(3R)-2,6-dioxopiperidin-3-yl]phenyl}piperidin-4-yl)acetaldehyde (8.11 mg, 0.0258 mmol) in DMA (0.30 mL) and then added N,N-diisopropylethylamine (12.28 μL, 0.01 g, 0.0703 mmol) and sodium triacetoxyborohydride (7.45 mg, 0.0352 mmol) and stirred for 2 h. The reaction was purified directly by RP-HPLC to yield 4-(difluoromethoxy)-N-{1-[2-(1-{4-[(3R)-2,6-dioxopiperidin-3-yl]phenyl}piperidin-4-yl)ethyl]piperidin-4-yl}-N-ethyl-1-[6-(2-hydroxyphenyl)pyridazin-4-yl]piperidine-4-carboxamide (0.0086 g, 44.75%). LCMS: C₄₂H₅₃F₂N₇O₅ requires: 773.4, found: m/z=774.3 [M+H]⁺.

### Example 108

### (1RS&,5SR&,8RS&)—N-{1-[2-(1-{4-[(3RS)-2,6-DIOXOPIPERIDIN-3-YL]-3,5-DIFLUOROPHENYL}PIPERIDIN-4-YL)ETHYL]PIPERIDIN-4-YL}-3-[6-(2-HYDROXYPHENYL)PYRIDAZIN-4-YL]-N—(PROPAN-2-YL)-3-AZABICYCLO[3.2.1]OCTANE-8-CARBOXAMIDE

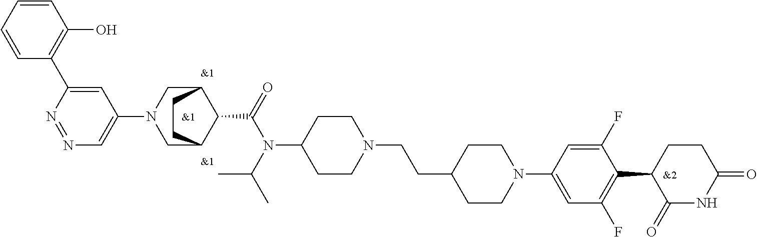 [SMILES: CC(C)N(C(=O)[C@H]1[C@@H]2CC[C@H]1CN(c1cnnc(-c3ccccc3O)c1)C2)C1CCN(CCC2CCN(c3cc(F)c([C@H]4CCC(=O)NC4=O)c(F)c3)CC2)CC1]

### Step 1: tert-butyl 4-[N-isopropyl(1R,5S,8S)-3-{6-[2-(methoxymethoxy)phenyl]pyridazin-4-yl}-3-azabicyclo[3.2.1]octane-8-amido]piperidine-1-carboxylate

To a solution of (1R,5S,8S)-3-{6-[2-(methoxymethoxy)phenyl]pyridazin-4-yl}-3-azabicyclo[3.2.1]octane-8-carboxylic acid (45.00 mg, 0.1218 mmol) in DMA (1.00 mL) was added tert-butyl 4-(isopropylamino)piperidine-1-carboxylate (44.28 mg, 0.1827 mmol) and N,N-diisopropylethylamine (63.65 μL, 0.05 g, 0.3654 mmol). [(dimethylamino)({[1,2,3]triazolo[4,5-b]pyridin-3-yloxy})methylidene]dimethylazanium; hexafluoro-lambda5-phosphanuide (55.58 mg, 0.1462 mmol) was added. Stirred at rt for 2 h, then added more N,N-diisopropylethylamine (63.65 μL, 0.05 g, 0.3654 mmol) and HATU (55 mg) and stirred overnight. Added more HATU (55 mg) and continued stirring at 40° C. overnight. Added more HATU, then added more amine (133 mg) and heated to 40° C. Purified directly by RP-HPLC to yield tert-butyl 4-[N-isopropyl(1R,5S,8S)-3-{6-[2-(methoxymethoxy)phenyl]pyridazin-4-yl}-3-azabicyclo[3.2.1]octane-8-amido]piperidine-1-carboxylate (0.023 g, 26.68%). LCMS: C₃₃H₄₇N₅O₅ requires: 593.4, found: m/z=594.2 [M+H]⁺.

### Step 2: (1R,5S,8S)-3-[6-(2-hydroxyphenyl)pyridazin-4-yl]-N-isopropyl-N-(piperidin-4-yl)-3-azabicyclo[3.2.1]octane-8-carboxamide

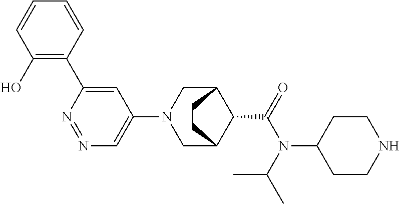 [SMILES: CC(C)N(C(=O)[C@H]1[C@@H]2CC[C@H]1CN(c1cnnc(-c3ccccc3O)c1)C2)C1CCNCC1]

(1R,5S,8S)-3-[6-(2-hydroxyphenyl)pyridazin-4-yl]-N-isopropyl-N-(piperidin-4-yl)-3-azabicyclo[3.2.1]octane-8-carboxamide was prepared as described for Example 107 step 2. LCMS: C₂₆H₃₅N₅O₂ requires: 449.3, found: m/z=450.2 [M+H]⁺.

### Step: 3 Synthesis of the Title Compound

The title compound was synthesized as described for Example 153 step 5, using (1R,5S,8S)-3-[6-(2-hydroxyphenyl)pyridazin-4-yl]-N-isopropyl-N-(piperidin-4-yl)-3-azabicyclo[3.2.1]octane-8-carboxamide hydrochloride and rac-2-(1-{4-[(3R)-2,6-dioxopiperidin-3-yl]-3,5-difluorophenyl}piperidin-4-yl)acetaldehyde to yield (1RS&,5SR&,8RS&)—N-{1-[2-(1-{4-[(3RS)-2,6-dioxopiperidin-3-yl]-3,5-difluorophenyl}piperidin-4-yl)ethyl]piperidin-4-yl}-3-[6-(2-hydroxyphenyl)pyridazin-4-yl]-N-isopropyl-3-azabicyclo[3.2.1]octane-8-carboxamide (0.0041 g, 37.04%). LCMS: C₄₄H₅₅F₂N₇O₄ requires: 783.4, found: m/z=784.3 [M+H]⁺.

### Example 109

### RAC-N-{1-[2-(1-{4-[(3R)-2,6-DIOXOPIPERIDIN-3-YL]-3-FLUOROPHENYL}PIPERIDIN-4-YL)ETHYL]PIPERIDIN-4-YL}-4-ETHOXY-N-ETHYL-1-[6-(2-HYDROXYPHENYL)PYRIDAZIN-4-YL]PIPERIDINE-4-CARBOXAMIDE

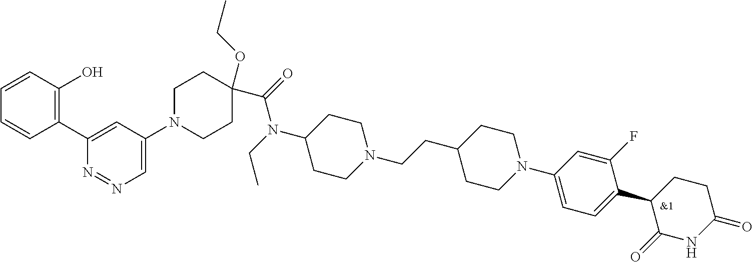 [SMILES: CCOC1(C(=O)N(CC)C2CCN(CCC3CCN(c4ccc([C@H]5CCC(=O)NC5=O)c(F)c4)CC3)CC2)CCN(c2cnnc(-c3ccccc3O)c2)CC1]

### Step 1: tert-butyl 4-(N-ethyl-4-ethoxy-1-{6-[2-(methoxymethoxy)phenyl]pyridazin-4-yl}piperidine-4-amido)piperidine-1-carboxylate

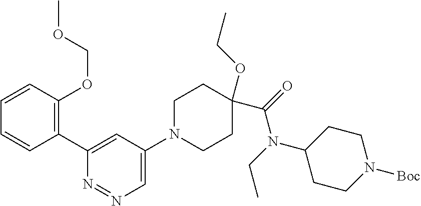 [SMILES: CCOC1(C(=O)N(CC)C2CCN(C(=O)OC(C)(C)C)CC2)CCN(c2cnnc(-c3ccccc3OCOC)c2)CC1]

Tert-butyl 4-(N-ethyl-4-ethoxy-1-{6-[2-(methoxymethoxy)phenyl]pyridazin-4-yl}piperidine-4-amido)piperidine-1-carboxylate was prepared as described for EXAMPLE 107 step 1, using 4-ethoxy-1-{6-[2-(methoxymethoxy)phenyl]pyridazin-4-yl}piperidine-4-carboxylic acid to yield tert-butyl 4-(N-ethyl-4-ethoxy-1-{6-[2-(methoxymethoxy)phenyl]pyridazin-4-yl}piperidine-4-amido)piperidine-1-carboxylate (0.053 g, 34.35%). LCMS: C₃₂H₄₇N₅O₆ requires: 597.4, found: m/z=598.4 [M+H]⁺.

### Step 2: 4-ethoxy-N-ethyl-1-[6-(2-hydroxyphenyl)pyridazin-4-yl]-N-(piperidin-4-yl)piperidine-4-carboxamide

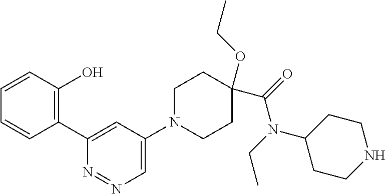 [SMILES: CCOC1(C(=O)N(CC)C2CCNCC2)CCN(c2cnnc(-c3ccccc3O)c2)CC1]

4-ethoxy-N-ethyl-1-[6-(2-hydroxyphenyl)pyridazin-4-yl]-N-(piperidin-4-yl)piperidine-4-carboxamide was prepared as described for Example 107 step 2. LCMS: C₂₅H₃₅N₅O₃ requires: 453.3, found: m/z=454.4 [M+H]⁺.

### Step 3: Synthesis of the Title Compound

Combined 4-ethoxy-N-ethyl-1-[6-(2-hydroxyphenyl)pyridazin-4-yl]-N-(piperidin-4-yl)piperidine-4-carboxamide hydrochloride (5.00 mg, 0.0102 mmol) and rac-2-(1-{4-[(3R)-2,6-dioxopiperidin-3-yl]-3-fluorophenyl}piperidin-4-yl)acetaldehyde (3.73 mg, 0.0112 mmol) in DMA (0.30 mL) and then added sodium triacetoxyborohydride (3.24 mg, 0.0153 mmol) and stirred for 2 h, then purified directly by HTP to yield rac-N-{1-[2-(1-{4-[(3R)-2,6-dioxopiperidin-3-yl]-3-fluorophenyl}piperidin-4-yl)ethyl]piperidin-4-yl}-4-ethoxy-N-ethyl-1-[6-(2-hydroxyphenyl)pyridazin-4-yl]piperidine-4-carboxamide (0.0046 g, 54.59%). LCMS: C₄₃H₅₆FN₇O₅ requires: 769.4, found: m/z=770.3 [M+H]⁺.

### Example 110

### RAC-4-(DIFLUOROMETHOXY)—N-{1-[2-(1-{4-[(3R)-2,6-DIOXOPIPERIDIN-3-YL]-3-FLUOROPHENYL}PIPERIDIN-4-YL)ETHYL]PIPERIDIN-4-YL}-N-ETHYL-1-[6-(2-HYDROXYPHENYL)PYRIDAZIN-4-YL]PIPERIDINE-4-CARBOXAMIDE

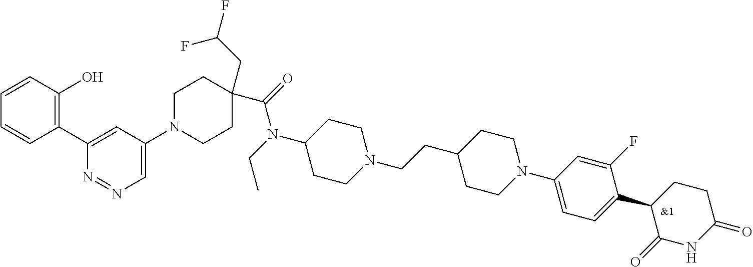 [SMILES: CCN(C(=O)C1(CC(F)F)CCN(c2cnnc(-c3ccccc3O)c2)CC1)C1CCN(CCC2CCN(c3ccc([C@H]4CCC(=O)NC4=O)c(F)c3)CC2)CC1]

The title compound was synthesized as described for Example 107 step 3, using rac-2-(1-{4-[(3R)-2,6-dioxopiperidin-3-yl]-3-fluorophenyl}piperidin-4-yl)acetaldehyde to yield rac-4-(difluoromethoxy)-N-{1-[2-(1-{4-[(3R)-2,6-dioxopiperidin-3-yl]-3-fluorophenyl}piperidin-4-yl)ethyl]piperidin-4-yl}-N-ethyl-1-[6-(2-hydroxyphenyl)pyridazin-4-yl]piperidine-4-carboxamide (0.0026 g, 47.81%). LCMS: C₄₂H₅₂F₃N₇O₅ requires: 791.4, found: m/z=792.3 [M+H]⁺.

### Example 111

### RAC-N-{1-[2-(1-{4-[(3R)-2,6-DIOXOPIPERIDIN-3-YL]PHENYL}-4-METHOXYPIPERIDIN-4-YL)ETHYL]PIPERIDIN-4-YL}-N-ETHYL-1-[6-(2-HYDROXYPHENYL)PYRIDAZIN-4-YL]-4-METHOXYPIPERIDINE-4-CARBOXAMIDE

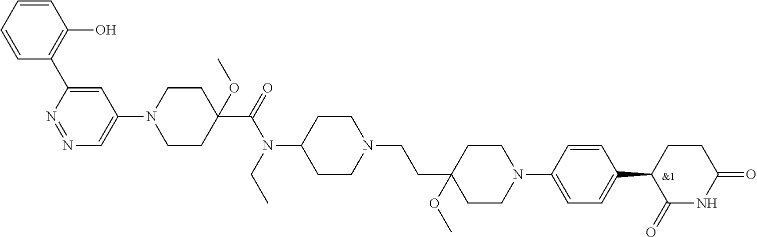 [SMILES: CCN(C(=O)C1(OC)CCN(c2cnnc(-c3ccccc3O)c2)CC1)C1CCN(CCC2(OC)CCN(c3ccc([C@H]4CCC(=O)NC4=O)cc3)CC2)CC1]

The title compound was synthesized as described for Example 107 step 3, using rac-2-(1-{4-[(3R)-2,6-dioxopiperidin-3-yl]phenyl}-4-methoxypiperidin-4-yl)acetaldehyde to yield rac-N-{1-[2-(1-{4-[(3R)-2,6-dioxopiperidin-3-yl]phenyl}-4-methoxypiperidin-4-yl)ethyl]piperidin-4-yl}-N-ethyl-1-[6-(2-hydroxyphenyl)pyridazin-4-yl]-4-methoxypiperidine-4-carboxamide (0.0113 g, 39.49%). LCMS: C₄₃H₅₇N₇O₆ requires: 767.4, found: m/z=768.2 [M+H]⁺.

### Example 112

### RAC-N-{1-[2-(1-{4-[(3R)-2,6-DIOXOPIPERIDIN-3-YL]-3-FLUOROPHENYL}-4-FLUOROPIPERIDIN-4-YL)ETHYL]PIPERIDIN-4-YL}-N-ETHYL-1-[6-(2-HYDROXYPHENYL)PYRIDAZIN-4-YL]-4-METHOXYPIPERIDINE-4-CARBOXAMIDE

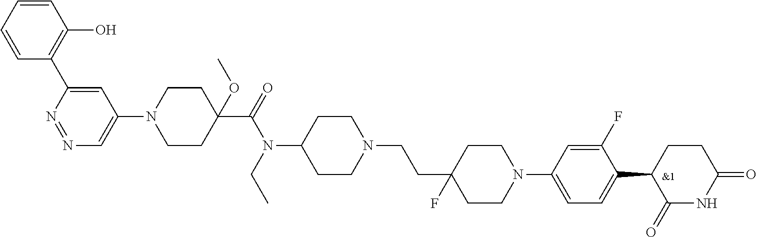 [SMILES: CCN(C(=O)C1(OC)CCN(c2cnnc(-c3ccccc3O)c2)CC1)C1CCN(CCC2(F)CCN(c3ccc([C@H]4CCC(=O)NC4=O)c(F)c3)CC2)CC1]

The title compound was synthesized as described for Example 107 step 3, using rac-2-(1-{4-[(3R)-2,6-dioxopiperidin-3-yl]-3-fluorophenyl}-4-fluoropiperidin-4-yl)acetaldehyde to yield rac-N-{1-[2-(1-{4-[(3R)-2,6-dioxopiperidin-3-yl]-3-fluorophenyl}-4-fluoropiperidin-4-yl)ethyl]piperidin-4-yl}-N-ethyl-1-[6-(2-hydroxyphenyl)pyridazin-4-yl]-4-methoxypiperidine-4-carboxamide (0.0063 g, 24.38%). LCMS: C₄₂H₅₃F₂N₇O₅ requires: 773.4, found: m/z=774.4 [M+H]⁺.

### Example 113

### (1RS&,5SR&,8RS&)—N-{1-[2-(1-{1-[(3RS)-2,6-DIOXOPIPERIDIN-3-YL]-3-METHYL-2-OXO-2,3-DIHYDRO-1H-1,3-BENZODIAZOL-4-YL}PIPERIDIN-4-YL)ETHYL]PIPERIDIN-4-YL}-3-[6-(2-HYDROXYPHENYL)PYRIDAZIN-4-YL]-N-(2-METHYLPROPYL)-3-AZABICYCLO[3.2.1]OCTANE-8-CARBOXAMIDE

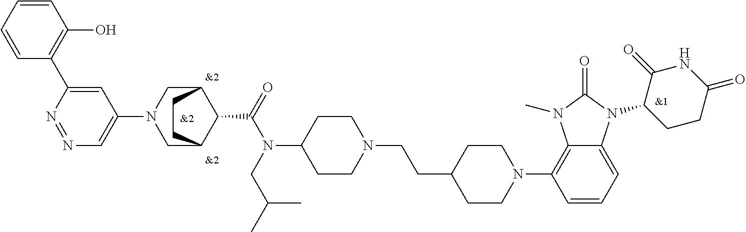 [SMILES: CC(C)CN(C(=O)[C@H]1[C@@H]2CC[C@H]1CN(c1cnnc(-c3ccccc3O)c1)C2)C1CCN(CCC2CCN(c3cccc4c3n(C)c(=O)n4[C@H]3CCC(=O)NC3=O)CC2)CC1]

### Step 1: tert-butyl 4-[N-(2-methylpropyl)(1R,5S,8S)-3-{6-[2-(methoxymethoxy)phenyl]pyridazin-4-yl}-3-azabicyclo[3.2.1]octane-8-amido]piperidine-1-carboxylate

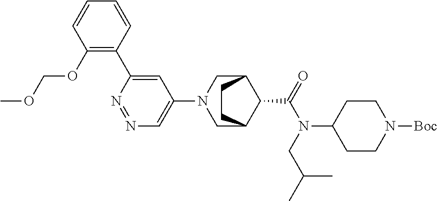 [SMILES: COCOc1ccccc1-c1cc(N2C[C@H]3CC[C@@H](C2)[C@H]3C(=O)N(CC(C)C)C2CCN(C(=O)OC(C)(C)C)CC2)cnn1]

To a solution of (1R,5S,8S)-3-{6-[2-(methoxymethoxy)phenyl]pyridazin-4-yl}-3-azabicyclo[3.2.1]octane-8-carboxylic acid (45.00 mg, 0.1218 mmol) in DMA (1.00 mL) was added tert-butyl 4-[(2-methylpropyl)amino]piperidine-1-carboxylate (34.35 mg, 0.1340 mmol) and N,N-diisopropylethylamine (106.09 μL, 0.08 g, 0.6091 mmol). [(dimethylamino)({[1,2,3]triazolo[4,5-b]pyridin-3-yloxy})methylidene]dimethylazanium; hexafluoro-lambda5-phosphanuide (55.58 mg, 0.1462 mmol) was added. Stirred at rt for 2 h, then added more HATU (55 mg) and stirred overnight. Purified directly by RP-HPLC to yield tert-butyl 4-[N-(2-methylpropyl)(1R,5S,8S)-3-{6-[2-(methoxymethoxy)phenyl]pyridazin-4-yl}-3-azabicyclo[3.2.1]octane-8-amido]piperidine-1-carboxylate (0.0496 g, 56.41%). LCMS: C₃₄H₄₉N₅O₅ requires: 607.4, found: m/z=608.4 [M+H]⁺.

### Step 2: (1R,5S,8S)-3-[6-(2-hydroxyphenyl)pyridazin-4-yl]-N-(2-methylpropyl)-N-(piperidin-4-yl)-3-azabicyclo[3.2.1]octane-8-carboxamide

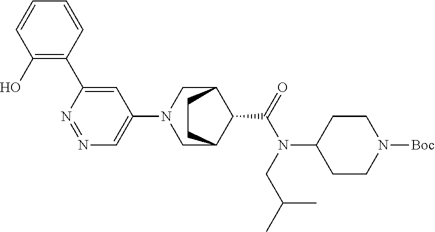 [SMILES: CC(C)CN(C(=O)[C@H]1[C@@H]2CC[C@H]1CN(c1cnnc(-c3ccccc3O)c1)C2)C1CCN(C(=O)OC(C)(C)C)CC1]

(1R,5S,8S)-3-[6-(2-hydroxyphenyl)pyridazin-4-yl]-N-(2-methylpropyl)-N-(piperidin-4-yl)-3-azabicyclo[3.2.1]octane-8-carboxamide was prepared as described for Example 107 step 2. LCMS: C₂₇H₃₇N₅O₂ requires: 463.3, found: m/z=464.2 [M+H]⁺.

### Step: 3 Synthesis of the Title Compound

The title compound was synthesized as described for Example 153 step 5, using (1R,5S,8S)-3-[6-(2-hydroxyphenyl)pyridazin-4-yl]-N-(2-methylpropyl)-N-(piperidin-4-yl)-3-azabicyclo[3.2.1]octane-8-carboxamide hydrochloride and rac-2-(1-{1-[(3R)-2,6-dioxopiperidin-3-yl]-3-methyl-2-oxo-1,3-benzodiazol-4-yl}piperidin-4-yl)acetaldehyde to yield (1RS&,5SR&,8RS&)—N-{1-[2-(1-{1-[(3RS)-2,6-dioxopiperidin-3-yl]-3-methyl-2-oxo-1,3-benzodiazol-4-yl}piperidin-4-yl)ethyl]piperidin-4-yl}-3-[6-(2-hydroxyphenyl)pyridazin-4-yl]-N-(2-methylpropyl)-3-azabicyclo[3.2.1]octane-8-carboxamide (0.0101 g, 54.73%). LCMS: C₄₇H₁₁N₉O₅ requires: 831.5, found: m/z=832.4 [M+H]⁺.

### Example 114

### N—((RS)-1-((SR)-8-(4-((R)-2,6-DIOXOPIPERIDIN-3-YL)PHENYL)-1-OXA-8-AZASPIRO[4.5]DECAN-3-YL)-3,3-DIFLUOROPIPERIDIN-4-YL)-N-ETHYL-1-(6-(2-HYDROXYPHENYL)PYRIDAZIN-4-YL)-4-METHOXYPIPERIDINE-4-CARBOXAMIDE

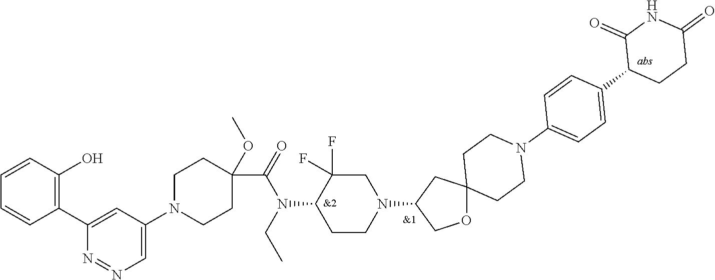 [SMILES: CCN(C(=O)C1(OC)CCN(c2cnnc(-c3ccccc3O)c2)CC1)[C@H]1CCN([C@H]2COC3(CCN(c4ccc([C@H]5CCC(=O)NC5=O)cc4)CC3)C2)CC1(F)F]

### Step 1: Synthesis of rac-(R)—N-(3,3-difluoropiperidin-4-yl)-N-ethyl-1-(6-(2-hydroxyphenyl)pyridazin-4-yl)-4-methoxypiperidine-4-carboxamide

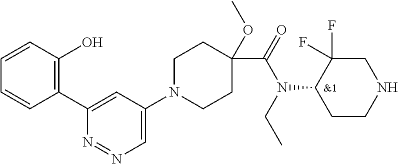 [SMILES: CCN(C(=O)C1(OC)CCN(c2cnnc(-c3ccccc3O)c2)CC1)[C@H]1CCNCC1(F)F]

The title compound was prepared as described for Example 89, Step 1, using tert-butyl 4-(ethylamino)-3,3-difluoropiperidine-1-carboxylate to afford the desired product as a yellow solid. LCMS: C₂₄H₃₁F₂N₅O₃ requires 475.2, found: m/z=476.4 [M+H]⁺.

### Step 2: Synthesis of the Title Compound

The title compound was prepared as described for Example 1, Step 2, using rac-(R)—N-(3,3-difluoropiperidin-4-yl)-N-ethyl-1-(6-(2-hydroxyphenyl)pyridazin-4-yl)-4-methoxypiperidine-4-carboxamide and (R)-3-(4-(3-oxo-1-oxa-8-azaspiro[4.5]decan-8-yl)phenyl)piperidine-2,6-dione to afford the title compound (7.2 mg, 20%). LCMS: C₄₃H₅₃F₂N₇O₆ requires 801.4, found: m/z=802.4 [M+H]⁺.

### Example 115

### RAC-N-(1-(((1R,4R)-4-(4-((R)-2,6-DIOXOPIPERIDIN-3-YL)PHENOXY)CYCLOHEXYL)METHYL)PIPERIDIN-4-YL)-N-ETHYL-1-(6-(2-HYDROXYPHENYL)PYRIDAZIN-4-YL)-4-METHOXYPIPERIDINE-4-CARBOXAMIDE

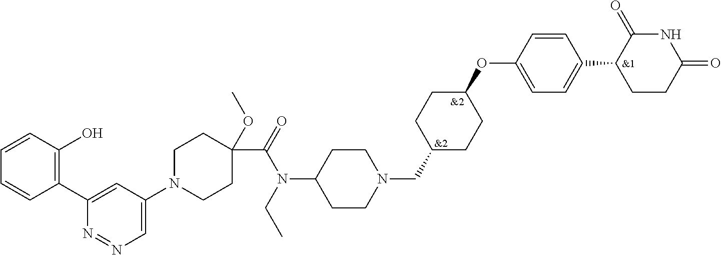 [SMILES: CCN(C(=O)C1(OC)CCN(c2cnnc(-c3ccccc3O)c2)CC1)C1CCN(C[C@H]2CC[C@H](Oc3ccc([C@H]4CCC(=O)NC4=O)cc3)CC2)CC1]

### Step 1: Synthesis of the Title Compound

The title compound was prepared as described for Example 1, Step 2, using N-ethyl-1-(6-(2-hydroxyphenyl)pyridazin-4-yl)-4-methoxy-N-(piperidin-4-yl)piperidine-4-carboxamide and rac-(1R,4r)-4-(4-((R)-2,6-dioxopiperidin-3-yl)phenoxy)cyclohexane-1-carbaldehyde (synthesized as described in WO2024254532 A1 2024-12-12) to afford the title compound (6.5 mg, 18%). LCMS: C₄₂H₅₄N₆O₆ requires 738.4, found: m/z=739.4 [M+H]⁺.

### Example 116

### (R)—N-(2,2-DIFLUOROETHYL)-N-(1-(2-(1-(4-(2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-4-(METHOXYMETHYL)PIPERIDINE-4-CARBOXAMIDE

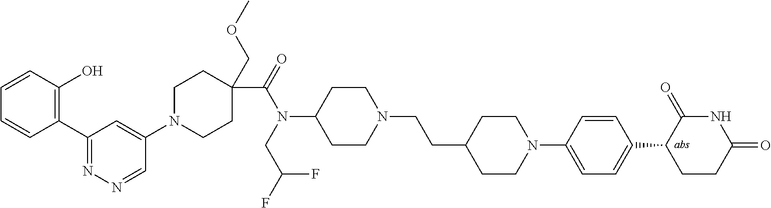 [SMILES: COCC1(C(=O)N(CC(F)F)C2CCN(CCC3CCN(c4ccc([C@H]5CCC(=O)NC5=O)cc4)CC3)CC2)CCN(c2cnnc(-c3ccccc3O)c2)CC1]

### Step 1: Synthesis of N-(2,2-difluoroethyl)-1-(6-(2-hydroxyphenyl)pyridazin-4-yl)-4-(methoxymethyl)-N-(piperidin-4-yl)piperidine-4-carboxamide hydrochloride

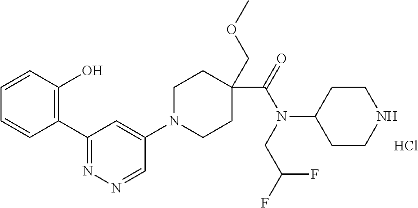 [SMILES: COCC1(C(=O)N(CC(F)F)C2CCNCC2)CCN(c2cnnc(-c3ccccc3O)c2)CC1 ; Cl]

The title compound was prepared as described for Example 2, Step 1 using 1-(6-(2-(methoxymethoxy)phenyl)pyridazin-4-yl)-4-(methoxymethyl)piperidine-4-carboxylic acid (Intermediate 13) and tert-butyl 4-((2,2-difluoroethyl)amino)piperidine-1-carboxylate to afford the title compound as a yellow oil. LCMS C₂₅H₃₃F₂N₅O₃ requires: 489.3, found m/z=490.3 [M+H]⁺.

### Step 2: Synthesis of the Title Compound

The title compound was prepared as described for Example 1, Step 2, using N-(2,2-difluoroethyl)-1-(6-(2-hydroxyphenyl)pyridazin-4-yl)-4-(methoxymethyl)-N-(piperidin-4-yl)piperidine-4-carboxamide hydrochloride (Example 116, Step 1) and (R)-2-(1-(4-(2,6-dioxopiperidin-3-yl)phenyl)piperidin-4-yl)acetaldehyde to afford the title compound (33.5 mgs, 78% yield) as a white solid. LCMS: C₄₃H₅₅F₂N₇O₅ requires: 787.4, found: m/z=788.3 [M+H]⁺.

### Example 117

### (1RS&,5SR&,8RS&)—N-{1-[2-(1-{1-[(3RS)-2,6-DIOXOPIPERIDIN-3-YL]-3-METHYL-2-OXO-2,3-DIHYDRO-1H-1,3-BENZODIAZOL-4-YL}PIPERIDIN-4-YL)ETHYL]PIPERIDIN-4-YL}-3-[6-(2-HYDROXYPHENYL)PYRIDAZIN-4-YL]-N-(3,3,3-TRIFLUOROPROPYL)-3-AZABICYCLO[3.2.1]OCTANE-8-CARBOXAMIDE

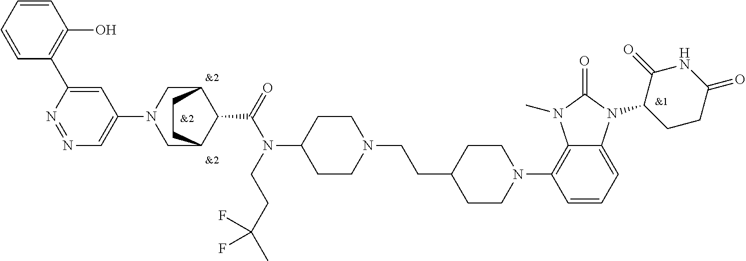 [SMILES: Cn1c(=O)n([C@H]2CCC(=O)NC2=O)c2cccc(N3CCC(CCN4CCC(N(CCC(C)(F)F)C(=O)[C@H]5[C@@H]6CC[C@H]5CN(c5cnnc(-c7ccccc7O)c5)C6)CC4)CC3)c21]

### Step 1: tert-butyl 4-[N-(3,3,3-trifluoropropyl)(1R,5S,8S)-3-{6-[2-(methoxymethoxy)phenyl]pyridazin-4-yl}-3-azabicyclo[3.2.1]octane-8-amido]piperidine-1-carboxylate

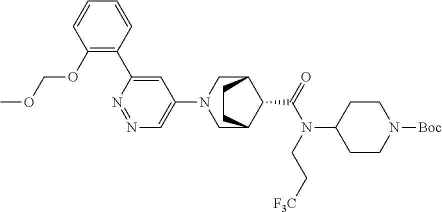 [SMILES: COCOc1ccccc1-c1cc(N2C[C@H]3CC[C@@H](C2)[C@H]3C(=O)N(CCC(F)(F)F)C2CCN(C(=O)OC(C)(C)C)CC2)cnn1]

To a solution of (1R,5S,8S)-3-{6-[2-(methoxymethoxy)phenyl]pyridazin-4-yl}-3-azabicyclo[3.2.1]octane-8-carboxylic acid (45.01 mg, 0.1218 mmol) in DMA (1.00 mL) was added tert-butyl 4-[(3,3,3-trifluoropropyl)amino]piperidine-1-carboxylate (39.72 mg, 0.1340 mmol) and N,N-diisopropylethylamine (63.67 μL, 0.05 g, 0.3655 mmol). [(dimethylamino)({[1,2,3]triazolo[4,5-b]pyridin-3-yloxy})methylidene]dimethylazanium; hexafluoro-lambda5-phosphanuide (55.59 mg, 0.1462 mmol) was added. Stirred at rt for 2 h, then added more N,N-diisopropylethylamine (63.67 μL, 0.05 g, 0.3655 mmol) and HATU (55 mg) and stirred overnight. Added more HATU (55 mg) and continued stirring at rt for 3 h, then at 40° C. overnight. Purified directly by RP-HPLC to yield tert-butyl 4-[N-(3,3,3-trifluoropropyl)(1R,5S,8S)-3-{6-[2-(methoxymethoxy)phenyl]pyridazin-4-yl}-3-azabicyclo[3.2.1]octane-8-amido]piperidine-1-carboxylate (0.049 g, 52.79%). LCMS: C₃₃H₄₄F₃N₅O₅ requires: 647.3, found: m/z=648.2 [M+H]⁺.

### Step 2: (1R,5S,8S)-3-[6-(2-hydroxyphenyl)pyridazin-4-yl]-N-(piperidin-4-yl)-N-(3,3,3-trifluoropropyl)-3-azabicyclo[3.2.1]octane-8-carboxamide

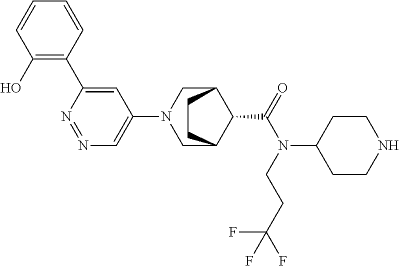 [SMILES: O=C([C@H]1[C@@H]2CC[C@H]1CN(c1cnnc(-c3ccccc3O)c1)C2)N(CCC(F)(F)F)C1CCNCC1]

(1R,5S,8S)-3-[6-(2-hydroxyphenyl)pyridazin-4-yl]-N-(piperidin-4-yl)-N-(3,3,3-trifluoropropyl)-3-azabicyclo[3.2.1]octane-8-carboxamide was prepared as described for Example 107 step 2. LCMS: C₂₆H₃₂F₃N₅O₂ requires: 503.3, found: m/z=504.2 [M+H]⁺.

### Step: 3 Synthesis of the Title Compound

The title compound was synthesized as described for Example 153 step 5, using (1R,5S,8S)-3-[6-(2-hydroxyphenyl)pyridazin-4-yl]-N-(piperidin-4-yl)-N-(3,3,3-trifluoropropyl)-3-azabicyclo[3.2.1]octane-8-carboxamide hydrochloride and rac-2-(1-{1-[(3R)-2,6-dioxopiperidin-3-yl]-3-methyl-2-oxo-1,3-benzodiazol-4-yl}piperidin-4-yl)acetaldehyde to yield (1RS&,5SR&,8RS&)—N-{1-[2-(1-{1-[(3RS)-2,6-dioxopiperidin-3-yl]-3-methyl-2-oxo-1,3-benzodiazol-4-yl}piperidin-4-yl)ethyl]piperidin-4-yl}-3-[6-(2-hydroxyphenyl)pyridazin-4-yl]-N-(3,3,3-trifluoropropyl)-3-azabicyclo[3.2.1]octane-8-carboxamide (0.0104 g, 61.18%). LCMS: C₄₆H₅₆F₃N₉O₅ requires: 871.4, found: m/z=872.3 [M+H]⁺.

### Example 118

### (1RS&,5SR&,8RS&)—N-{1-[2-(1-{4-[(3RS)-2,6-DIOXOPIPERIDIN-3-YL]-3,5-DIFLUOROPHENYL}PIPERIDIN-4-YL)ETHYL]PIPERIDIN-4-YL}-3-[6-(2-HYDROXYPHENYL)PYRIDAZIN-4-YL]-N-(3,3,3-TRIFLUOROPROPYL)-3-AZABICYCLO[3.2.1]OCTANE-8-CARBOXAMIDE

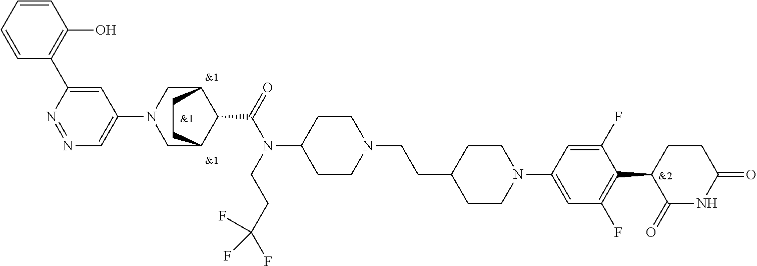 [SMILES: O=C1CC[C@H](c2c(F)cc(N3CCC(CCN4CCC(N(CCC(F)(F)F)C(=O)[C@H]5[C@@H]6CC[C@H]5CN(c5cnnc(-c7ccccc7O)c5)C6)CC4)CC3)cc2F)C(=O)N1]

The title compound was synthesized as described for Example 153 step 5, using (1R,5S,8S)-3-[6-(2-hydroxyphenyl)pyridazin-4-yl]-N-(piperidin-4-yl)-N-(3,3,3-trifluoropropyl)-3-azabicyclo[3.2.1]octane-8-carboxamide hydrochloride and rac-2-(1-{4-[(3R)-2,6-dioxopiperidin-3-yl]-3,5-difluorophenyl}piperidin-4-yl)acetaldehyde to yield (1RS&,5SR&,8RS&)—N-{1-[2-(1-{4-[(3RS)-2,6-dioxopiperidin-3-yl]-3,5-difluorophenyl}piperidin-4-yl)ethyl]piperidin-4-yl}-3-[6-(2-hydroxyphenyl)pyridazin-4-yl]-N-(3,3,3-trifluoropropyl)-3-azabicyclo[3.2.1]octane-8-carboxamide (0.0103 g, 62.92%). LCMS: C₄₄H₅₂F₅N₇O₄ requires: 837.4, found: m/z=838.3 [M+H]⁺.

### Example 119

### (S)—N-CYCLOPROPYL-N-(1-(2-(1-(4-(2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-4-(METHOXYMETHYL)PIPERIDINE-4-CARBOXAMIDE

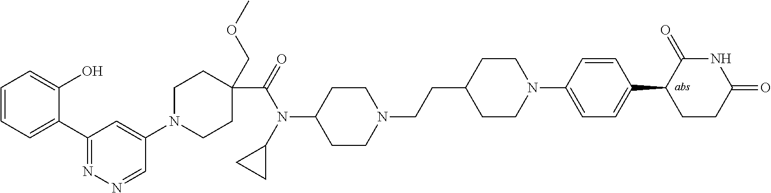 [SMILES: COCC1(C(=O)N(C2CC2)C2CCN(CCC3CCN(c4ccc([C@@H]5CCC(=O)NC5=O)cc4)CC3)CC2)CCN(c2cnnc(-c3ccccc3O)c2)CC1]

The title compound was synthesized as described for Example 59.

### Example 120

### (1RS,5SR,8RS)—N-{1-[2-(1-{4-[(3R)-2,6-DIOXOPIPERIDIN-3-YL]-3-FLUOROPHENYL}PIPERIDIN-4-YL)ETHYL]PIPERIDIN-4-YL}-3-[6-(2-HYDROXYPHENYL)PYRIDAZIN-4-YL]-N-(3,3,3-TRIFLUOROPROPYL)-3-AZABICYCLO[3.2.1]OCTANE-8-CARBOXAMIDE

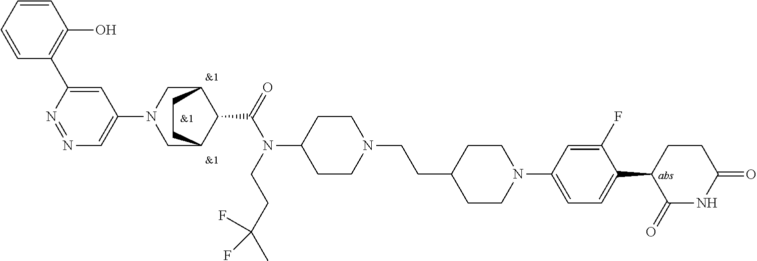 [SMILES: CC(F)(F)CCN(C(=O)[C@H]1[C@@H]2CC[C@H]1CN(c1cnnc(-c3ccccc3O)c1)C2)C1CCN(CCC2CCN(c3ccc([C@H]4CCC(=O)NC4=O)c(F)c3)CC2)CC1]

The title compound was synthesized as described for Example 153 step 5, using (1R,5S,8S)-3-[6-(2-hydroxyphenyl)pyridazin-4-yl]-N-(piperidin-4-yl)-N-(3,3,3-trifluoropropyl)-3-azabicyclo[3.2.1]octane-8-carboxamide hydrochloride and 2-(1-{4-[(3R)-2,6-dioxopiperidin-3-yl]-3-fluorophenyl}piperidin-4-yl)acetaldehyde to yield (1RS,5SR,8RS)—N-{1-[2-(1-{4-[(3R)-2,6-dioxopiperidin-3-yl]-3-fluorophenyl}piperidin-4-yl)ethyl]piperidin-4-yl}-3-[6-(2-hydroxyphenyl)pyridazin-4-yl]-N-(3,3,3-trifluoropropyl)-3-azabicyclo[3.2.1]octane-8-carboxamide (0.0105 g, 65.41%). LCMS: C₄₄H₅₃F₄N₇O₄ requires: 819.4, found: m/z=820.2 [M+H]⁺.

### Example 121

### RAC-N-(CYCLOPROPYLMETHYL)-N-{1-[2-(1-{5-[(3R)-2,6-DIOXOPIPERIDIN-3-YL]PYRIDIN-2-YL}PIPERIDIN-4-YL)ETHYL]PIPERIDIN-4-YL}-1-[6-(2-HYDROXYPHENYL)PYRIDAZIN-4-YL]-4-(METHOXYMETHYL)PIPERIDINE-4-CARBOXAMIDE

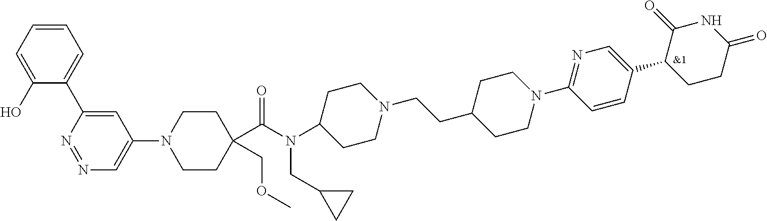 [SMILES: COCC1(C(=O)N(CC2CC2)C2CCN(CCC3CCN(c4ccc([C@H]5CCC(=O)NC5=O)cn4)CC3)CC2)CCN(c2cnnc(-c3ccccc3O)c2)CC1]

### Step 1: Synthesis of benzyl 4-[(cyclopropylmethyl)amino]piperidine-1-carboxylate

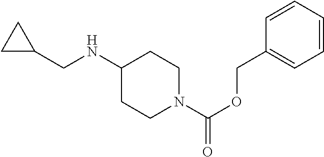 [SMILES: O=C(OCc1ccccc1)N1CCC(NCC2CC2)CC1]

To a mixture of benzyl 4-oxopiperidine-1-carboxylate (500 mg, 2.14 mmol) and 1-cyclopropylmethanamine (152 mg, 2.14 mmol) in DCM (5.00 mL) was added sodium triacetoxyborohydride (1.36 g, 6.43 mmol). After 2 hours, water was added. The mixture was extracted twice with DCM. The combined organic layers were concentrated. The crude residue was purified by flash chromatography on a 40 g column eluted with 0 to 20% MeOH/DCM to provide benzyl 4-[(cyclopropylmethyl)amino]piperidine-1-carboxylate (0.617 g, 100%). LCMS: C₁₇H₂₄N₂O₂ requires: 288.2, found: m/z=289.2 [M+H]⁺.

### Step 2: Synthesis of tert-butyl 4-({1-[(benzyloxy)carbonyl]piperidin-4-yl}(cyclopropylmethyl)carbamoyl)-4-(methoxymethyl)piperidine-1-carboxylate

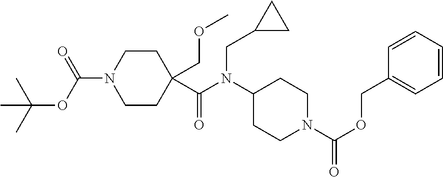 [SMILES: COCC1(C(=O)N(CC2CC2)C2CCN(C(=O)OCc3ccccc3)CC2)CCN(C(=O)OC(C)(C)C)CC1]

To a vial with 1-(tert-butoxycarbonyl)-4-(methoxymethyl)piperidine-4-carboxylic acid (250 mg, 0.915 mmol) in DCM (3.00 mL) was added benzyl 4-[(cyclopropylmethyl)amino]piperidine-1-carboxylate (340 mg, 1.18 mmol) and N,N-diisopropylethylamine (0.64 mL, 0.47 g, 3.66 mmol) followed by bis(2-oxo-1,3-oxazolidin-3-yl)phosphinoyl chloride (699 mg, 2.74 mmol). The mixture was heated at 40° C. for 90 minutes then overnight at room temp. The mixture was diluted with water and extracted 3×DCM. The combined organic layers were dried over Na₂SO₄ and concentrated. The crude residue was purified by flash chromatography on a 24 g column eluted with gradient elution to 70% EtOAc/heptane to provide tert-butyl 4-({1-[(benzyloxy)carbonyl]piperidin-4-yl}(cyclopropylmethyl)carbamoyl)-4-(methoxymethyl)piperidine-1-carboxylate (0.243 g, 49%). LCMS: C₃₀H₄₅N₃O₆ requires: 543.3, found: m/z=566.4 [M+Na]⁺.

### Step 3: Synthesis of benzyl 4-[N-(cyclopropylmethyl)-4-(methoxymethyl)piperidine-4-amido]piperidine-1-carboxylate

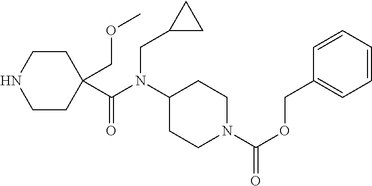 [SMILES: COCC1(C(=O)N(CC2CC2)C2CCN(C(=O)OCc3ccccc3)CC2)CCNCC1]

tert-butyl 4-({1-[(benzyloxy)carbonyl]piperidin-4-yl}(cyclopropylmethyl)carbamoyl)-4-(methoxymethyl)piperidine-1-carboxylate (243 mg, 0.447 mmol) was stirred in MeOH (2.00 mL) and 4M hydrogen chloride in dioxane solution (2.00 mL, 0.29 g, 8.00 mmol) for 40 minutes then was concentrated to provide benzyl 4-[N-(cyclopropylmethyl)-4-(methoxymethyl)piperidine-4-amido]piperidine-1-carboxylate (0.198 g, 100%). LCMS: C₂₅H₃₇N₃O₄ requires: 443.3, found: m/z=444.2 [M+H]⁺.

### Step 4: Synthesis of benzyl 4-[N-(cyclopropylmethyl)-1-(6-chloropyridazin-4-yl)-4-(methoxymethyl)piperidine-4-amido]piperidine-1-carboxylate

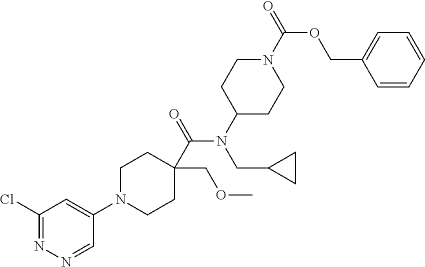 [SMILES: COCC1(C(=O)N(CC2CC2)C2CCN(C(=O)OCc3ccccc3)CC2)CCN(c2cnnc(Cl)c2)CC1]

A mixture of benzyl 4-[N-(cyclopropylmethyl)-4-(methoxymethyl)piperidine-4-amido]piperidine-1-carboxylate (198 mg, 0.447 mmol), 3,5-dichloropyridazine (76.2 mg, 0.512 mmol) and N,N-diisopropylethylamine (0.45 mL, 0.33 g, 2.56 mmol) in DMSO (1.50 mL) was heated in a 100° C. heating block for 30 minutes. 13 mL water was added. The mixture was extracted 3× ethyl acetate. The combined organic layers were dried over Na₂SO₄ and concentrated. The crude residue was purified by flash chromatography on a 24 g column eluted with gradient elution with 0 to 10% MeOH/EtOAc to provide benzyl 4-[N-(cyclopropylmethyl)-1-(6-chloropyridazin-4-yl)-4-(methoxymethyl)piperidine-4-amido]piperidine-1-carboxylate (0.185 g, 74%). LCMS: C₂₉H₃₈ClN₅O₄ requires: 555.3, found: m/z=556.2 [M+H]⁺.

### Step 5: Synthesis of benzyl 4-[N-(cyclopropylmethyl)-1-[6-(2-hydroxyphenyl)pyridazin-4-yl]-4-(methoxymethyl)piperidine-4-amido]piperidine-1-carboxylate

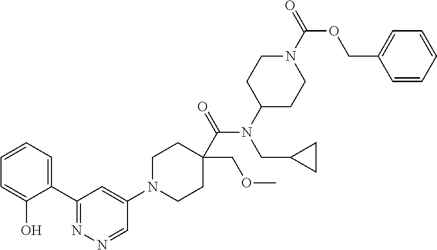 [SMILES: COCC1(C(=O)N(CC2CC2)C2CCN(C(=O)OCc3ccccc3)CC2)CCN(c2cnnc(-c3ccccc3O)c2)CC1]

To a vial with benzyl 4-[N-(cyclopropylmethyl)-1-(6-chloropyridazin-4-yl)-4-(methoxymethyl)piperidine-4-amido]piperidine-1-carboxylate (183 mg, 0.329 mmol), 2-hydroxyphenylboronic acid (59.0 mg, 0.428 mmol), and potassium carbonate (136 mg, 0.987 mmol) was added dioxane (4.50 mL) and water (1.00 mL). A vacuum was pulled until the mixture bubbled then the headspace was backfilled with argon for 5 cycles. tetrakis(triphenylphosphine)palladium(O) (38.0 mg, 0.0329 mmol) was added. A vacuum was pulled until the mixture bubbled then the headspace was backfilled with argon for 5 cycles. The mixture was heated in a 100° C. heating block overnight. Water was added and the mixture was extracted 3×EtOAc. The combined organic layers were dried over Na₂SO₄ and concentrated. The crude residue was purified by flash chromatography on a 24 g column eluted with 0 to 100% EtOAc/heptane to provide benzyl 4-[N-(cyclopropylmethyl)-1-[6-(2-hydroxyphenyl)pyridazin-4-yl]-4-(methoxymethyl)piperidine-4-amido]piperidine-1-carboxylate (0.147 g, 73%). LCMS: C₃₅H₄₃N₅O₅ requires: 613.3, found: m/z=556.2 [M+H]⁺.

### Step 6: Synthesis of N-(cyclopropylmethyl)-1-[6-(2-hydroxyphenyl)pyridazin-4-yl]-4-(methoxymethyl)-N-(piperidin-4-yl)piperidine-4-carboxamide

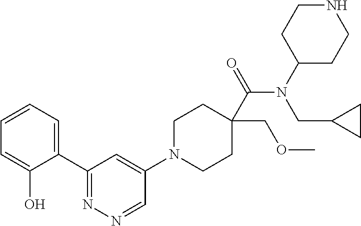 [SMILES: COCC1(C(=O)N(CC2CC2)C2CCNCC2)CCN(c2cnnc(-c3ccccc3O)c2)CC1]

To a mixture of benzyl 4-[N-(cyclopropylmethyl)-1-[6-(2-hydroxyphenyl)pyridazin-4-yl]-4-(methoxymethyl)piperidine-4-amido]piperidine-1-carboxylate (147 mg, 0.240 mmol) in THF (2.00 mL) and EtOH (2.00 mL) was added 10% Pd/C (15.00 mg). The mixture was stirred under a balloon of H₂ overnight. The mixture was diluted with THF and filtered through a pad of celite then concentrated to provide N-(cyclopropylmethyl)-1-[6-(2-hydroxyphenyl)pyridazin-4-yl]-4-(methoxymethyl)-N-(piperidin-4-yl)piperidine-4-carboxamide (0.115 g, 100%). LCMS: C₂₇H₃₇N₅O₃ requires: 479.3, found: m/z=480.4 [M+H]⁺.

### Step 7: Synthesis of the Title Compound

To a vial with N-(cyclopropylmethyl)-1-[6-(2-hydroxyphenyl)pyridazin-4-yl]-4-(methoxymethyl)-N-(piperidin-4-yl)piperidine-4-carboxamide (36.0 mg, 0.0751 mmol) and rac-2-(1-{5-[(3R)-2,6-dioxopiperidin-3-yl]pyridin-2-yl}piperidin-4-yl)acetaldehyde (23.7 mg, 0.0751 mmol) in DMA (1.00 mL) was added sodium triacetoxyborohydride (19.1 mg, 0.0901 mmol). After 1 hour, the mixture was filtered with a syringe filter then purified by reverse phase HPLC to provide the title compound (0.0039 g, 6%). LCMS: C₄₄H₅₈N₈O₅ requires: 778.5, found: m/z=779.3 [M+H]⁺.

### Example 122

### RAC-N-(CYCLOPROPYLMETHYL)-N-{1-[2-(1-{4-[(3R)-2,6-DIOXOPIPERIDIN-3-YL]-3-FLUOROPHENYL}PIPERIDIN-4-YL)ETHYL]PIPERIDIN-4-YL}-1-[6-(2-HYDROXYPHENYL)PYRIDAZIN-4-YL]-4-(METHOXYMETHYL)PIPERIDINE-4-CARBOXAMIDE

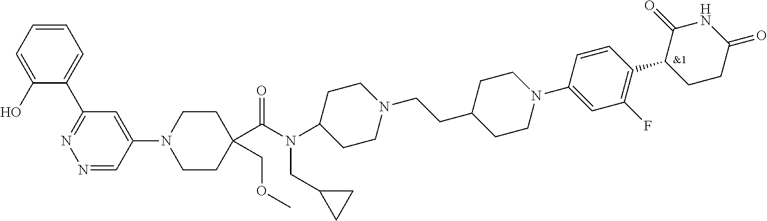 [SMILES: COCC1(C(=O)N(CC2CC2)C2CCN(CCC3CCN(c4ccc([C@H]5CCC(=O)NC5=O)c(F)c4)CC3)CC2)CCN(c2cnnc(-c3ccccc3O)c2)CC1]

The title compound was prepared as described for Example 121, Step 7, using rac-2-(1-{4-[(3R)-2,6-dioxopiperidin-3-yl]-3-fluorophenyl}piperidin-4-yl)acetaldehyde (24.3 mg, 0.0730 mmol) to afford the title compound (7.4 mg, 12% yield). LCMS: C₄₅H₅₈FN₇O₅ requires 795.5, found m/z=796.3 [M+H]⁺.

### Example 123

### RAC-N-CYCLOPROPYL-N-{1-[2-(1-{4-[(3R)-2,6-DIOXOPIPERIDIN-3-YL]PHENYL}PIPERIDIN-4-YL)ETHYL]PIPERIDIN-4-YL}-1-[6-(2-HYDROXYPHENYL)PYRIDAZIN-4-YL]-4-(METHOXYMETHYL)PIPERIDINE-4-CARBOXAMIDE

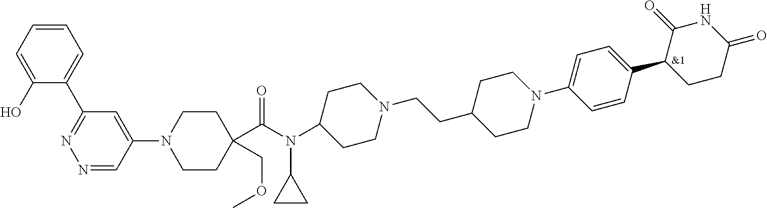 [SMILES: COCC1(C(=O)N(C2CC2)C2CCN(CCC3CCN(c4ccc([C@@H]5CCC(=O)NC5=O)cc4)CC3)CC2)CCN(c2cnnc(-c3ccccc3O)c2)CC1]

### Step 1: Synthesis of benzyl 4-(cyclopropylamino)piperidine-1-carboxylate

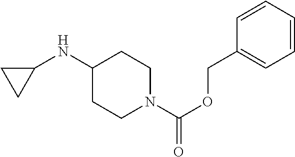 [SMILES: O=C(OCc1ccccc1)N1CCC(NC2CC2)CC1]

The title compound was prepared as described for Example 121 Step 1 using aminocyclopropane (269 mg, 4.72 mmol) to afford benzyl 4-(cyclopropylamino)piperidine-1-carboxylate (1.12 g, 95%). LCMS: C₁₆H₂₂N₂O₂ requires 274.2, found m/z=275.2 [M+H]⁺.

### Step 2: Synthesis of tert-butyl 4-({1-[(benzyloxy)carbonyl]piperidin-4-yl}(cyclopropyl)carbamoyl)-4-(methoxymethyl)piperidine-1-carboxylate

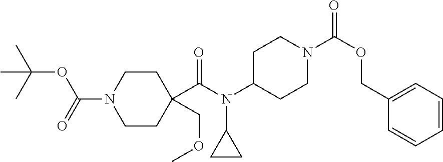 [SMILES: COCC1(C(=O)N(C2CC2)C2CCN(C(=O)OCc3ccccc3)CC2)CCN(C(=O)OC(C)(C)C)CC1]

The title compound was prepared as described for Example 121 Step 2 using benzyl 4-(cyclopropylamino)piperidine-1-carboxylate (1.21 g, 4.41 mmol) to afford tert-butyl 4-({1-[(benzyloxy)carbonyl]piperidin-4-yl}(cyclopropyl)carbamoyl)-4-(methoxymethyl)piperidine-1-carboxylate (0.899 g, 52%). LCMS: C₂₉H₄₃N₃O₆ requires 529.3, found m/z=552.4 [M+Na]+.

### Step 3: Synthesis of benzyl 4-[N-cyclopropyl-4-(methoxymethyl)piperidine-4-amido]piperidine-1-carboxylate

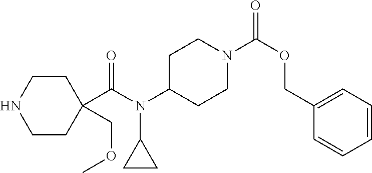 [SMILES: COCC1(C(=O)N(C2CC2)C2CCN(C(=O)OCc3ccccc3)CC2)CCNCC1]

The title compound was prepared as described for Example 121 Step 3 using tert-butyl 4-({1-[(benzyloxy)carbonyl]piperidin-4-yl}(cyclopropyl)carbamoyl)-4-(methoxymethyl)piperidine-1-carboxylate (899 mg, 1.70 mmol) to afford benzyl 4-[N-cyclopropyl-4-(methoxymethyl)piperidine-4-amido]piperidine-1-carboxylate (0.729 g, 100%). LCMS: C₂₄H₃₅N₃O₄ requires 429.3, found m/z=430.4 [M+H]⁺.

### Step 4: Synthesis of benzyl 4-[N-cyclopropyl-1-(6-chloropyridazin-4-yl)-4-(methoxymethyl)piperidine-4-amido]piperidine-1-carboxylate

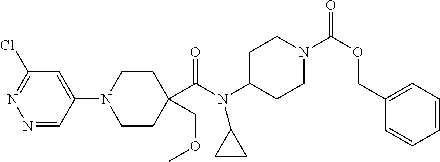 [SMILES: COCC1(C(=O)N(C2CC2)C2CCN(C(=O)OCc3ccccc3)CC2)CCN(c2cnnc(Cl)c2)CC1]

The title compound was prepared as described for Example 121 Step 4 using benzyl 4-[N-cyclopropyl-4-(methoxymethyl)piperidine-4-amido]piperidine-1-carboxylate (729 mg, 1.70 mmol) to afford benzyl 4-[N-cyclopropyl-1-(6-chloropyridazin-4-yl)-4-(methoxymethyl)piperidine-4-amido]piperidine-1-carboxylate (0.760 g, 82%). LCMS: C₂₈H₃₆ClN₅O₄ requires 541.3, found m/z=542.4 [M+H]⁺.

### Step 5: Synthesis of benzyl 4-{N-cyclopropyl-1-[6-(2-hydroxyphenyl)pyridazin-4-yl]-4-(methoxymethyl)piperidine-4-amido}piperidine-1-carboxylate

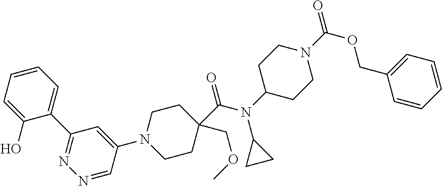 [SMILES: COCC1(C(=O)N(C2CC2)C2CCN(C(=O)OCc3ccccc3)CC2)CCN(c2cnnc(-c3ccccc3O)c2)CC1]

The title compound was prepared as described for Example 121 Step 5 using benzyl 4-[N-cyclopropyl-1-(6-chloropyridazin-4-yl)-4-(methoxymethyl)piperidine-4-amido]piperidine-1-carboxylate (749 mg, 1.38 mmol) to afford benzyl 4-{N-cyclopropyl-1-[6-(2-hydroxyphenyl)pyridazin-4-yl]-4-(methoxymethyl)piperidine-4-amido}piperidine-1-carboxylate (0.580 g, 70%). LCMS: C₃₄H₄₁N₅O₅ requires 599.3, found m/z=600.4 [M+H]⁺.

### Step 6: Synthesis of N-cyclopropyl-1-[6-(2-hydroxyphenyl)pyridazin-4-yl]-4-(methoxymethyl)-N-(piperidin-4-yl)piperidine-4-carboxamide

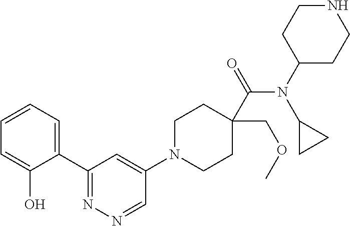 [SMILES: COCC1(C(=O)N(C2CCNCC2)C2CC2)CCN(c2cnnc(-c3ccccc3O)c2)CC1]

The title compound was prepared as described for Example 121 Step 6 using benzyl 4-{N-cyclopropyl-1-[6-(2-hydroxyphenyl)pyridazin-4-yl]-4-(methoxymethyl)piperidine-4-amido}piperidine-1-carboxylate (561 mg, 0.935 mmol) to afford N-cyclopropyl-1-[6-(2-hydroxyphenyl)pyridazin-4-yl]-4-(methoxymethyl)-N-(piperidin-4-yl)piperidine-4-carboxamide (0.435 g, 100%). LCMS: C₂₆H₃₅N₅O₃ requires 465.3, found m/z=466.4 [M+H]⁺.

### Step 7: Synthesis of the Title Compound

To a vial with N-cyclopropyl-1-[6-(2-hydroxyphenyl)pyridazin-4-yl]-4-(methoxymethyl)-N-(piperidin-4-yl)piperidine-4-carboxamide (82 mg, 0.176 mmol) and rac-2-(1-{4-[(3R)-2,6-dioxopiperidin-3-yl]phenyl}piperidin-4-yl)acetaldehyde (55.4 mg, 0.176 mmol) in acetonitrile (1.50 mL) and MeOH (0.50 mL) was added sodium triacetoxyborohydride (112 mg, 0.528 mmol). After 30 minutes, water was added and the mixture was extracted 3×DCM. The combined organic layers were concentrated then purified by prep TLC eluted with 10% MeOH/DCM to provide rac-N-cyclopropyl-N-{1-[2-(1-{4-[(3R)-2,6-dioxopiperidin-3-yl]phenyl}piperidin-4-yl)ethyl]piperidin-4-yl}-1-[6-(2-hydroxyphenyl)pyridazin-4-yl]-4-(methoxymethyl)piperidine-4-carboxamide (0.0644 g, 48%). ¹H NMR (500 MHz, DMSO) δ 14.66 (s, 1H), 10.77 (s, 1H), 8.88 (d, J=3.0 Hz, 1H), 8.10 (dd, J=8.4, 1.7 Hz, 1H), 7.48 (d, J=2.9 Hz, 1H), 7.33 (ddd, J=8.5, 7.1, 1.5 Hz, 1H), 7.06-7.00 (m, 2H), 6.96-6.82 (m, 4H), 3.99-3.82 (m, 3H), 3.72 (dd, J=11.0, 4.9 Hz, 1H), 3.64 (d, J=12.2 Hz, 2H), 3.49 (s, 2H), 3.26 (s, 3H), 3.15 (t, J=11.6 Hz, 2H), 2.96 (s, 2H), 2.63 (ddd, J=12.4, 9.1, 6.7 Hz, 3H), 2.45-2.21 (m, 6H), 2.22-1.96 (m, 5H), 1.94-1.83 (m, 1H), 1.81-1.61 (m, 4H), 1.55 (d, J=11.4 Hz, 2H), 1.47-1.30 (m, 3H), 1.30-1.16 (m, 2H), 0.81-0.73 (m, 2H), 0.55-0.42 (m, 2H). LCMS: C₄₄H₅₇N₇O₅ requires: 763.4, found: m/z=764.4 [M+H]⁺.

### Example 124

### N-{1-[2-(1-{4-[(3R)-2,6-DIOXOPIPERIDIN-3-YL]PHENYL}PIPERIDIN-4-YL)ETHYL]PIPERIDIN-4-YL}-1-[6-(2-HYDROXYPHENYL)PYRIDAZIN-4-YL]-4-(METHOXYMETHYL)-N-(2-METHYLPROPYL)PIPERIDINE-4-CARBOXAMIDE

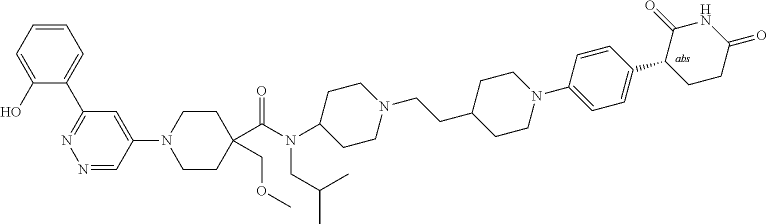 [SMILES: COCC1(C(=O)N(CC(C)C)C2CCN(CCC3CCN(c4ccc([C@H]5CCC(=O)NC5=O)cc4)CC3)CC2)CCN(c2cnnc(-c3ccccc3O)c2)CC1]

### Step 1: Synthesis of benzyl 4-[(2-methylpropyl)amino]piperidine-1-carboxylate

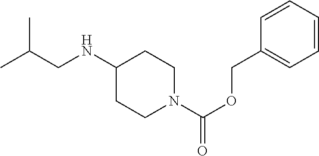 [SMILES: CC(C)CNC1CCN(C(=O)OCc2ccccc2)CC1]

The title compound was prepared as described for Example 121 Step 1 using isobutylamine (157 mg, 2.14 mmol) to afford benzyl 4-[(2-methylpropyl)amino]piperidine-1-carboxylate (0.479 g, 77%). LCMS: C₁₇H₂₆N₂O₂ requires: 290.2, found: m/z=291.2 [M+H]⁺.

### Step 2: Synthesis of tert-butyl 4-({1-[(benzyloxy)carbonyl]piperidin-4-yl}(2-methylpropyl)carbamoyl)-4-(methoxymethyl)piperidine-1-carboxylate

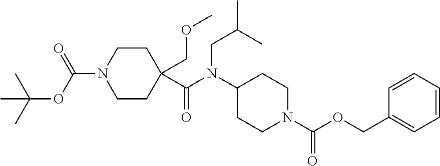 [SMILES: COCC1(C(=O)N(CC(C)C)C2CCN(C(=O)OCc3ccccc3)CC2)CCN(C(=O)OC(C)(C)C)CC1]

The title compound was prepared as described for Example 121 Step 2 using benzyl 4-[(2-methylpropyl)amino]piperidine-1-carboxylate (479 mg, 1.65 mmol) to afford tert-butyl 4-({1-[(benzyloxy)carbonyl]piperidin-4-yl}(2-methylpropyl)carbamoyl)-4-(methoxymethyl)piperidine-1-carboxylate (0.505 g, 72%). LCMS: C₃₀H₄₇N₃O₆ requires: 545.4, found: m/z=568.4 [M+Na]⁺.

### Step 3: Synthesis of benzyl 4-[N-(2-methylpropyl)-4-(methoxymethyl)piperidine-4-amido]piperidine-1-carboxylate

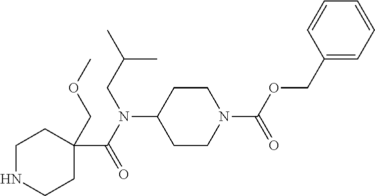 [SMILES: COCC1(C(=O)N(CC(C)C)C2CCN(C(=O)OCc3ccccc3)CC2)CCNCC1]

The title compound was prepared as described for Example 121 Step 3 using tert-butyl 4-({1-[(benzyloxy)carbonyl]piperidin-4-yl}(2-methylpropyl)carbamoyl)-4-(methoxymethyl)piperidine-1-carboxylate (505.00 mg, 0.9254 mmol) to afford benzyl 4-[N-(2-methylpropyl)-4-(methoxymethyl)piperidine-4-amido]piperidine-1-carboxylate (0.412 g, 100%). LCMS: C₂₅H₃₉N₃O₄ requires: 445.3, found: m/z=446.4 [M+H]⁺.

### Step 4: Synthesis of benzyl 4-[N-(2-methylpropyl)-1-(6-chloropyridazin-4-yl)-4-(methoxymethyl)piperidine-4-amido]piperidine-1-carboxylate

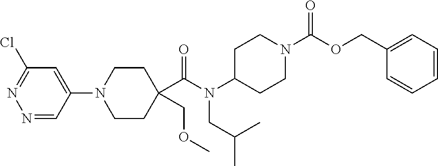 [SMILES: COCC1(C(=O)N(CC(C)C)C2CCN(C(=O)OCc3ccccc3)CC2)CCN(c2cnnc(Cl)c2)CC1]

The title compound was prepared as described for Example 121 Step 4 using benzyl 4-[N-(2-methylpropyl)-4-(methoxymethyl)piperidine-4-amido]piperidine-1-carboxylate (412 mg, 0.926 mmol) to afford benzyl 4-[N-(2-methylpropyl)-1-(6-chloropyridazin-4-yl)-4-(methoxymethyl)piperidine-4-amido]piperidine-1-carboxylate (0.277 g, 54%). LCMS: C₂₉H_{40° C.}lN₅O₄ requires: 557.3, found: m/z=558.4 [M+H]⁺.

### Step 5: Synthesis of benzyl 4-[N-(2-methylpropyl)-1-[6-(2-hydroxyphenyl)pyridazin-4-yl]-4-(methoxymethyl)piperidine-4-amido]piperidine-1-carboxylate

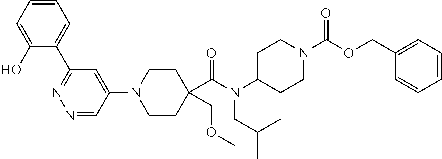 [SMILES: COCC1(C(=O)N(CC(C)C)C2CCN(C(=O)OCc3ccccc3)CC2)CCN(c2cnnc(-c3ccccc3O)c2)CC1]

The title compound was prepared as described for Example 121 Step 5 using benzyl 4-[N-(2-methylpropyl)-1-(6-chloropyridazin-4-yl)-4-(methoxymethyl)piperidine-4-amido]piperidine-1-carboxylate (267 mg, 0.478 mmol) to afford benzyl 4-[N-(2-methylpropyl)-1-[6-(2-hydroxyphenyl)pyridazin-4-yl]-4-(methoxymethyl)piperidine-4-amido]piperidine-1-carboxylate (0.243 g, 82%). LCMS: C₃₅H₄₅N₅O₅ requires: 615.3, found: m/z=616.4 [M+H]⁺.

### Step 6: Synthesis of 1-[6-(2-hydroxyphenyl)pyridazin-4-yl]-4-(methoxymethyl)-N-(2-methylpropyl)-N-(piperidin-4-yl)piperidine-4-carboxamide

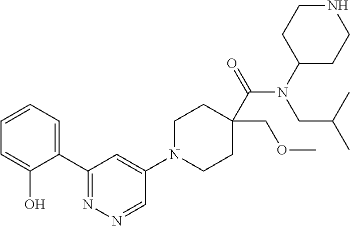 [SMILES: COCC1(C(=O)N(CC(C)C)C2CCNCC2)CCN(c2cnnc(-c3ccccc3O)c2)CC1]

The title compound was prepared as described for Example 121 Step 6 using benzyl 4-[N-(2-methylpropyl)-1-[6-(2-hydroxyphenyl)pyridazin-4-yl]-4-(methoxymethyl)piperidine-4-amido]piperidine-1-carboxylate (243 mg, 0.395 mmol) to afford 1-[6-(2-hydroxyphenyl)pyridazin-4-yl]-4-(methoxymethyl)-N-(2-methylpropyl)-N-(piperidin-4-yl)piperidine-4-carboxamide (0.190 g, 100%). LCMS: C₂₇H₃₉N₅O₃ requires: 481.3, found: m/z=482.3 [M+H]⁺.

### Step 7: Synthesis of the Title Compound

The title compound was prepared as described for Example 123 Step 7 using 1-[6-(2-hydroxyphenyl)pyridazin-4-yl]-4-(methoxymethyl)-N-(2-methylpropyl)-N-(piperidin-4-yl)piperidine-4-carboxamide (66.0 mg, 0.137 mmol) and 2-(1-{4-[(3R)-2,6-dioxopiperidin-3-yl]phenyl}piperidin-4-yl)acetaldehyde (43.1 mg, 0.137 mmol) to afford N-{1-[2-(1-{4-[(3R)-2,6-dioxopiperidin-3-yl]phenyl}piperidin-4-yl)ethyl]piperidin-4-yl}-1-[6-(2-hydroxyphenyl)pyridazin-4-yl]-4-(methoxymethyl)-N-(2-methylpropyl)piperidine-4-carboxamide (0.0112 g, 9%). ¹H NMR (500 MHz, DMSO) δ 14.64 (s, 1H), 10.78 (s, 1H), 9.02 (s, 1H), 8.88 (d, J=2.9 Hz, 1H), 8.19-7.97 (m, 1H), 7.47 (d, J=2.9 Hz, 1H), 7.34 (td, J=7.6, 1.6 Hz, 1H), 7.14-6.96 (m, 2H), 6.96-6.79 (m, 4H), 4.20 (s, 1H), 3.87 (d, J=13.9 Hz, 2H), 3.80-3.63 (m, 3H), 3.63-3.45 (m, 4H), 3.28-3.02 (m, 8H), 3.00-2.80 (m, 2H), 2.72-2.56 (m, 3H), 2.49-2.41 (m, 1H), 2.27 (d, J=13.8 Hz, 2H), 2.19-1.89 (m, 5H), 1.88-1.67 (m, 7H), 1.62 (s, 2H), 1.48 (s, 1H), 1.39-1.15 (m, 2H), 0.81 (d, J=6.7 Hz, 6H). LCMS: C₄₅H₆₁N₇O₅ requires: 779.5, found: m/z=780.3 [M+H]⁺.

### Example 125

### RAC-(1R,5S,8S)—N-(CYCLOPROPYLMETHYL)-N-(1-(2-(1-(4-((R)-2,6-DIOXOPIPERIDIN-3-YL)-3,5-DIFLUOROPHENYL)PIPERIDIN-4-YL)ETHYL)PIPERIDIN-4-YL)-3-(6-(2-HYDROXYPHENYL)PYRIDAZIN-4-YL)-3-AZABICYCLO[3.2.1]OCTANE-8-CARBOXAMIDE

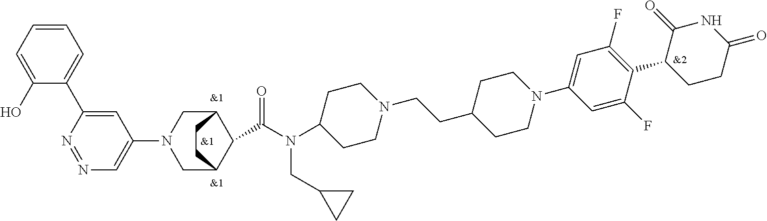 [SMILES: O=C1CC[C@H](c2c(F)cc(N3CCC(CCN4CCC(N(CC5CC5)C(=O)[C@H]5[C@@H]6CC[C@H]5CN(c5cnnc(-c7ccccc7O)c5)C6)CC4)CC3)cc2F)C(=O)N1]

### Step 1: Synthesis of the Title Compound

N-(cyclopropylmethyl)-3-[6-(2-hydroxyphenyl)pyridazin-4-yl]-N-(piperidin-4-yl)-3-azabicyclo[3.2.1]octane-8-carboxamide hydrochloride (8.00 mg, 0.0150 mmol) and 2-(1-{4-[(3R)-2,6-dioxopiperidin-3-yl]-3,5-difluorophenyl}piperidin-4-yl)acetaldehyde (5.18 mg, 0.0165 mmol) were added to a vial and DMA (0.50 mL) was added. Sodium triacetoxyborohydride (9.52 mg, 0.0449 mmol) was then added the reaction was stirred at RT for 30 mins and checked by LCMS. Once complete, the reaction was directly purified by RP—FC to afford the title compound (2.5 mg, 19.5%) as a formate salt. LCMS: C₄₅H₅₅F₂N₇O₄ requires: 795.4, found: m/z=796.3 [M+H]⁺.

### Example 126

### RAC-N-(2,2-DIMETHYLPROPYL)-N-{1-[2-(1-{4-[(3R)-2,6-DIOXOPIPERIDIN-3-YL]-3-FLUOROPHENYL}PIPERIDIN-4-YL)ETHYL]PIPERIDIN-4-YL}-1-[6-(2-HYDROXYPHENYL)PYRIDAZIN-4-YL]-4-(METHOXYMETHYL)PIPERIDINE-4-CARBOXAMIDE

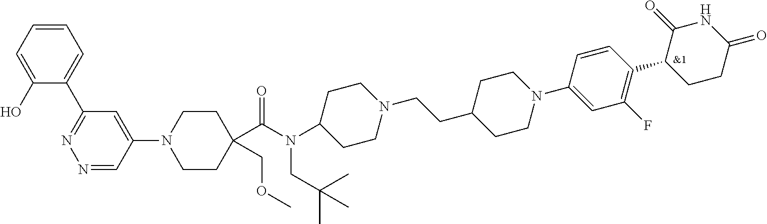 [SMILES: COCC1(C(=O)N(CC(C)(C)C)C2CCN(CCC3CCN(c4ccc([C@H]5CCC(=O)NC5=O)c(F)c4)CC3)CC2)CCN(c2cnnc(-c3ccccc3O)c2)CC1]

### Step 1: Synthesis of benzyl 4-[(2,2-dimethylpropyl)amino]piperidine-1-carboxylate

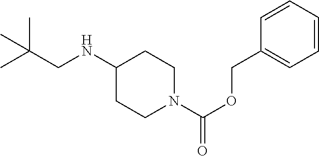 [SMILES: CC(C)(C)CNC1CCN(C(=O)OCc2ccccc2)CC1]

The title compound was prepared as described for Example 121 Step 1 using neopentylamine (187 mg, 2.14 mmol) to afford benzyl 4-[(2,2-dimethylpropyl)amino]piperidine-1-carboxylate (0.652 g, 100%). LCMS: C₁₈H₂₈N₂O₂ requires: 304.2, found: m/z=305.2 [M+H]⁺.

### Step 2: Synthesis of tert-butyl 4-({1-[(benzyloxy)carbonyl]piperidin-4-yl}(2,2-dimethylpropyl)carbamoyl)-4-(methoxymethyl)piperidine-1-carboxylate

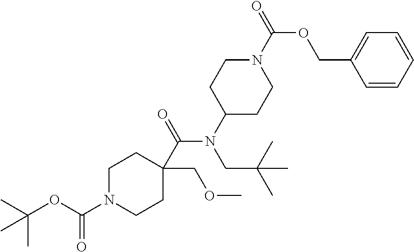 [SMILES: COCC1(C(=O)N(CC(C)(C)C)C2CCN(C(=O)OCc3ccccc3)CC2)CCN(C(=O)OC(C)(C)C)CC1]

The title compound was prepared as described for Example 121 Step 2 using benzyl 4-[(2,2-dimethylpropyl)amino]piperidine-1-carboxylate (652 mg, 2.14 mmol) to afford tert-butyl 4-({1-[(benzyloxy)carbonyl]piperidin-4-yl}(2,2-dimethylpropyl)carbamoyl)-4-(methoxymethyl)piperidine-1-carboxylate (0.510 g, 55%). LCMS: C₃₁H₄₉N₃O₆ requires: 559.4, found: m/z=582.4 [M+Na]⁺.

### Step 3: Synthesis of benzyl 4-[N-(2,2-dimethylpropyl)-4-(methoxymethyl)piperidine-4-amido]piperidine-1-carboxylate

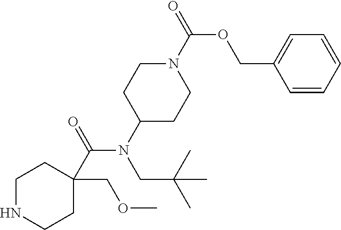 [SMILES: COCC1(C(=O)N(CC(C)(C)C)C2CCN(C(=O)OCc3ccccc3)CC2)CCNCC1]

The title compound was prepared as described for Example 121 Step 3 using tert-butyl 4-({1-[(benzyloxy)carbonyl]piperidin-4-yl}(2,2-dimethylpropyl)carbamoyl)-4-(methoxymethyl)piperidine-1-carboxylate (510 mg, 0.911 mmol) to afford benzyl 4-[N-(2,2-dimethylpropyl)-4-(methoxymethyl)piperidine-4-amido]piperidine-1-carboxylate (0.419 g, 100%). LCMS: C₂₆H₄₁N₃O₄ requires: 459.3, found: m/z=460.4 [M+H]⁺.

### Step 4: Synthesis of benzyl 4-[N-(2,2-dimethylpropyl)-1-(6-chloropyridazin-4-yl)-4-(methoxymethyl)piperidine-4-amido]piperidine-1-carboxylate

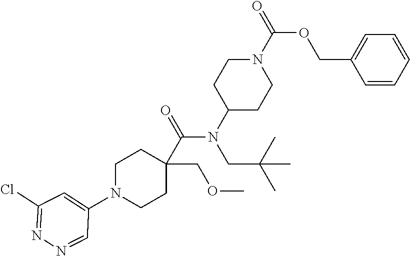 [SMILES: COCC1(C(=O)N(CC(C)(C)C)C2CCN(C(=O)OCc3ccccc3)CC2)CCN(c2cnnc(Cl)c2)CC1]

The title compound was prepare as described for Example 121 Step 4 using benzyl 4-[N-(2,2-dimethylpropyl)-4-(methoxymethyl)piperidine-4-amido]piperidine-1-carboxylate (419 mg, 0.911 mmol) to afford benzyl 4-[N-(2,2-dimethylpropyl)-1-(6-chloropyridazin-4-yl)-4-(methoxymethyl)piperidine-4-amido]piperidine-1-carboxylate (0.141 g, 15%). LCMS: C₃₀H₄₂ClN₅O₄ requires: 571.3, found: m/z=572.4 [M+H]⁺.

### Step 5: Synthesis of 4-[N-(2,2-dimethylpropyl)-1-[6-(2-hydroxyphenyl)pyridazin-4-yl]-4-(methoxymethyl)piperidine-4-amido]piperidine-1-carboxylate

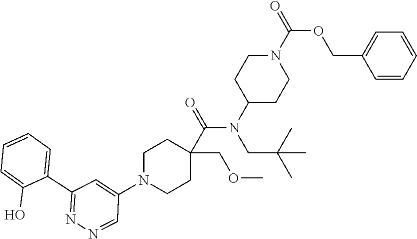 [SMILES: COCC1(C(=O)N(CC(C)(C)C)C2CCN(C(=O)OCc3ccccc3)CC2)CCN(c2cnnc(-c3ccccc3O)c2)CC1]

The title compound was prepared as described for Example 121 Step 5 using benzyl 4-[N-(2,2-dimethylpropyl)-1-(6-chloropyridazin-4-yl)-4-(methoxymethyl)piperidine-4-amido]piperidine-1-carboxylate (141 mg, 0.246 mmol) to afford 4-[N-(2,2-dimethylpropyl)-1-[6-(2-hydroxyphenyl)pyridazin-4-yl]-4-(methoxymethyl)piperidine-4-amido]piperidine-1-carboxylate (0.111 g, 72%). LCMS: C₃₆H₄₇N₅O₅ requires: 629.4, found: m/z=630.4 [M+H]⁺.

### Step 6: Synthesis of N-(2,2-dimethylpropyl)-1-[6-(2-hydroxyphenyl)pyridazin-4-yl]-4-(methoxymethyl)-N-(piperidin-4-yl)piperidine-4-carboxamide

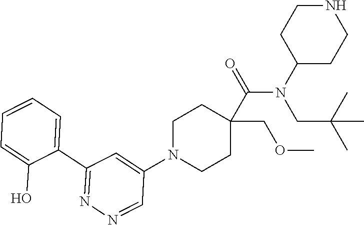 [SMILES: COCC1(C(=O)N(CC(C)(C)C)C2CCNCC2)CCN(c2cnnc(-c3ccccc3O)c2)CC1]

The title compound was prepared as described for Example 121 Step 6 using benzyl 4-[N-(2,2-dimethylpropyl)-1-[6-(2-hydroxyphenyl)pyridazin-4-yl]-4-(methoxymethyl)piperidine-4-amido]piperidine-1-carboxylate (110 mg, 0.175 mmol) to afford N-(2,2-dimethylpropyl)-1-[6-(2-hydroxyphenyl)pyridazin-4-yl]-4-(methoxymethyl)-N-(piperidin-4-yl)piperidine-4-carboxamide (0.087 g, 100%). LCMS: C₂₈H₄₁N₅O₃ requires: 495.3, found: m/z=496.4 [M+H]⁺.

### Step 7: Synthesis of the Title Compound

The title compound was prepared as described for Example 123 Step 7 using N-(2,2-dimethylpropyl)-1-[6-(2-hydroxyphenyl)pyridazin-4-yl]-4-(methoxymethyl)-N-(piperidin-4-yl)piperidine-4-carboxamide (33.0 mg, 0.0666 mmol) and rac-2-(1-{4-[(3R)-2,6-dioxopiperidin-3-yl]-3-fluorophenyl}piperidin-4-yl)acetaldehyde (22.1 mg, 0.0666 mmol) to provide rac-N-(2,2-dimethylpropyl)-N-{1-[2-(1-{4-[(3R)-2,6-dioxopiperidin-3-yl]-3-fluorophenyl}piperidin-4-yl)ethyl]piperidin-4-yl}-1-[6-(2-hydroxyphenyl)pyridazin-4-yl]-4-(methoxymethyl)piperidine-4-carboxamide (0.0088 g, 16%). ¹H NMR (500 MHz, DMSO) δ 14.67 (s, 1H), 10.80 (s, 1H), 8.89 (d, J=2.8 Hz, 1H), 8.16-8.05 (m, 1H), 7.48 (d, J=2.9 Hz, 1H), 7.33 (td, J=7.7, 1.6 Hz, 1H), 7.07 (t, J=8.7 Hz, 1H), 6.99-6.89 (m, 2H), 6.77-6.63 (m, 2H), 3.88 (dd, J=12.4, 4.9 Hz, 1H), 3.85-3.76 (m, 2H), 3.70 (d, J=12.4 Hz, 2H), 3.54 (s, 2H), 3.45 (t, J=10.4 Hz, 2H), 3.25 (s, 3H), 3.18-2.79 (m, 3H), 2.76-2.60 (m, 3H), 2.45-2.07 (m, 6H), 2.07-1.90 (m, 2H), 1.90-1.66 (m, 6H), 1.59-1.48 (m, 2H), 1.48-1.32 (m, 3H), 1.32-1.14 (m, 5H), 0.83 (s, 9H). LCMS: C₄₆H₆₂FN₇O₅ requires: 811.5, found: m/z=812.4 [M+H]⁺.

### Example 127

### N-[(2R)-BUTAN-2-YL]-N-{1-[2-(1-{4-[(3R)-2,6-DIOXOPIPERIDIN-3-YL]PHENYL}PIPERIDIN-4-YL)ETHYL]PIPERIDIN-4-YL}-1-[6-(2-HYDROXYPHENYL)PYRIDAZIN-4-YL]-4-(METHOXYMETHYL)PIPERIDINE-4-CARBOXAMIDE

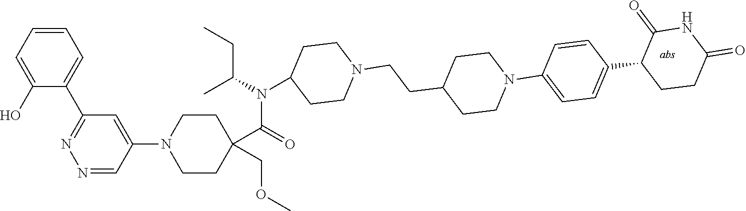 [SMILES: CC[C@@H](C)N(C(=O)C1(COC)CCN(c2cnnc(-c3ccccc3O)c2)CC1)C1CCN(CCC2CCN(c3ccc([C@H]4CCC(=O)NC4=O)cc3)CC2)CC1]

### Step 1: Synthesis of benzyl 4-[(2R)-butan-2-ylamino]piperidine-1-carboxylate

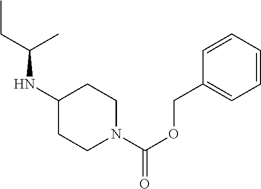 [SMILES: CC[C@@H](C)NC1CCN(C(=O)OCc2ccccc2)CC1]

The title compound was prepared as described for Example 121 Step 1 using (R)-2-butanamine (157 mg, 2.14 mmol) to afford benzyl 4-[(2R)-butan-2-ylamino]piperidine-1-carboxylate (0.616 g, 99%). LCMS: C₁₇H₂₆N₂O₂ requires: 290.2, found: m/z=291.2 [M+H]⁺.

### Step 2: Synthesis of tert-butyl 4-({1-[(benzyloxy)carbonyl]piperidin-4-yl}[(2R)-butan-2-yl]carbamoyl)-4-(methoxymethyl)piperidine-1-carboxylate

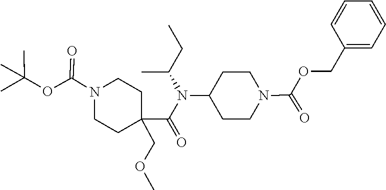 [SMILES: CC[C@@H](C)N(C(=O)C1(COC)CCN(C(=O)OC(C)(C)C)CC1)C1CCN(C(=O)OCc2ccccc2)CC1]

The title compound was prepared as described for Example 121 Step 2 using benzyl 4-[(2R)-butan-2-ylamino]piperidine-1-carboxylate (500 mg, 1.72 mmol) to afford tert-butyl 4-({1-[(benzyloxy)carbonyl]piperidin-4-yl}[(2R)-butan-2-yl]carbamoyl)-4-(methoxymethyl)piperidine-1-carboxylate (0.546 g, 58%). LCMS: C₃₀H₄₇N₃O₆ requires: 545.4, found: m/z=568.4 [M+Na]⁺.

### Step 3: Synthesis of benzyl 4-{N-[(2R)-butan-2-yl]-4-(methoxymethyl)piperidine-4-amido}piperidine-1-carboxylate

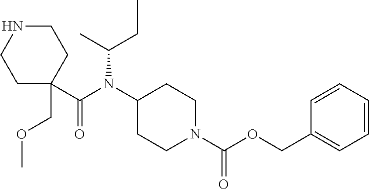 [SMILES: CC[C@@H](C)N(C(=O)C1(COC)CCNCC1)C1CCN(C(=O)OCc2ccccc2)CC1]

The title compound was prepared as described for Example 121 Step 3 using tert-butyl 4-({1-[(benzyloxy)carbonyl]piperidin-4-yl}[(2R)-butan-2-yl]carbamoyl)-4-(methoxymethyl)piperidine-1-carboxylate (546 mg, 1.00 mmol) to afford benzyl 4-{N-[(2R)-butan-2-yl]-4-(methoxymethyl)piperidine-4-amido}piperidine-1-carboxylate (0.445 g, 100%). LCMS: C₂₅H₃₉N₃O₄ requires: 445.3, found: m/z=446.4 [M+H]⁺.

### Step 4: Synthesis of benzyl 4-{N-[(2R)-butan-2-yl]-1-(6-chloropyridazin-4-yl)-4-(methoxymethyl)piperidine-4-amido}piperidine-1-carboxylate

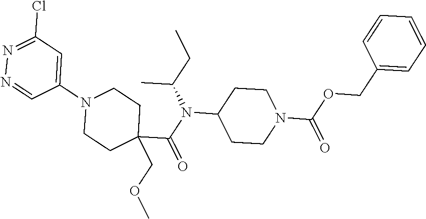 [SMILES: CC[C@@H](C)N(C(=O)C1(COC)CCN(c2cnnc(Cl)c2)CC1)C1CCN(C(=O)OCc2ccccc2)CC1]

The title compound was prepared as described for Example 121 Step 4 using benzyl 4-{N-[(2R)-butan-2-yl]-4-(methoxymethyl)piperidine-4-amido}piperidine-1-carboxylate (445 mg, 1.00 mmol) to afford benzyl 4-{N-[(2R)-butan-2-yl]-1-(6-chloropyridazin-4-yl)-4-(methoxymethyl)piperidine-4-amido}piperidine-1-carboxylate (0.120 g, 21%). LCMS: C₂₉H_{40° C.}N₅O₄ requires: 557.3, found: m/z=558.4 [M+H]⁺.

### Step 5: Synthesis of benzyl 4-{N-[(2R)-butan-2-yl]-1-[6-(2-hydroxyphenyl)pyridazin-4-yl]-4-(methoxymethyl)piperidine-4-amido}piperidine-1-carboxylate

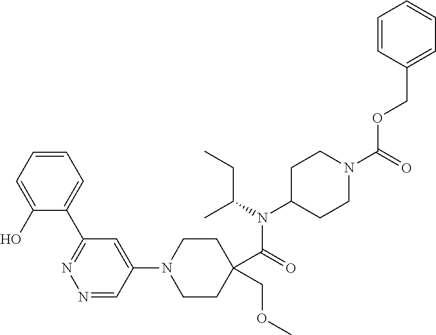 [SMILES: CC[C@@H](C)N(C(=O)C1(COC)CCN(c2cnnc(-c3ccccc3O)c2)CC1)C1CCN(C(=O)OCc2ccccc2)CC1]

The title compound was prepared as described for Example 121 Step 5 using benzyl 4-{N-[(2R)-butan-2-yl]-1-(6-chloropyridazin-4-yl)-4-(methoxymethyl)piperidine-4-amido}piperidine-1-carboxylate (118 mg, 0.211 mmol) to afford benzyl 4-{N-[(2R)-butan-2-yl]-1-[6-(2-hydroxyphenyl)pyridazin-4-yl]-4-(methoxymethyl)piperidine-4-amido}piperidine-1-carboxylate (0.096 g, 74%). LCMS: C₃₅H₄₅N₅O₅ requires: 615.3, found: m/z=616.4 [M+H]⁺.

### Step 6: Synthesis of N-[(2R)-butan-2-yl]-1-[6-(2-hydroxyphenyl)pyridazin-4-yl]-4-(methoxymethyl)-N-(piperidin-4-yl)piperidine-4-carboxamide

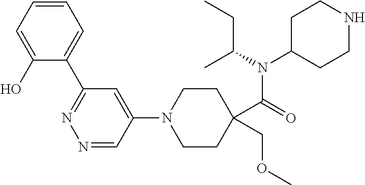 [SMILES: CC[C@@H](C)N(C(=O)C1(COC)CCN(c2cnnc(-c3ccccc3O)c2)CC1)C1CCNCC1]

The title compound was prepared as described for Example 121 Step 6 using benzyl 4-{N-[(2R)-butan-2-yl]-1-[6-(2-hydroxyphenyl)pyridazin-4-yl]-4-(methoxymethyl)piperidine-4-amido}piperidine-1-carboxylate (96.0 mg, 0.156 mmol) to afford N-[(2R)-butan-2-yl]-1-[6-(2-hydroxyphenyl)pyridazin-4-yl]-4-(methoxymethyl)-N-(piperidin-4-yl)piperidine-4-carboxamide (0.075 g, 100%). LCMS: C₂₇H₃₉N₅O₃ requires: 481.3, found: m/z=482.4 [M+H]⁺.

### Step 7: Synthesis of the Title Compound

The title compound was prepared as described for Example 123 Step 7 using N-[(2R)-butan-2-yl]-1-[6-(2-hydroxyphenyl)pyridazin-4-yl]-4-(methoxymethyl)-N-(piperidin-4-yl)piperidine-4-carboxamide (45.0 mg, 0.0934 mmol) and 2-(1-{4-[(3R)-2,6-dioxopiperidin-3-yl]phenyl}piperidin-4-yl)acetaldehyde (29.4 mg, 0.0934 mmol) to afford N-[(2R)-butan-2-yl]-N-{1-[2-(1-{4-[(3R)-2,6-dioxopiperidin-3-yl]phenyl}piperidin-4-yl)ethyl]piperidin-4-yl}-1-[6-(2-hydroxyphenyl)pyridazin-4-yl]-4-(methoxymethyl)piperidine-4-carboxamide (0.0184 g, 24%). ¹H NMR (500 MHz, DMSO) δ 14.69 (s, 1H), 10.79 (s, 1H), 8.88 (d, J=3.0 Hz, 1H), 8.21-8.01 (m, 1H), 7.48 (d, J=3.3 Hz, 1H), 7.33 (td, J=7.6, 1.6 Hz, 1H), 7.03 (d, J=8.2 Hz, 2H), 6.97-6.82 (m, 4H), 4.08-3.79 (m, 3H), 3.72 (dd, J=11.0, 4.9 Hz, 1H), 3.64 (d, J=11.9 Hz, 2H), 3.50 (s, 2H), 3.26 (s, 3H), 2.98 (d, J=11.4 Hz, 3H), 2.87-2.68 (m, 1H), 2.68-2.56 (m, 3H), 2.48-2.41 (m, 1H), 2.41-2.31 (m, 2H), 2.31-2.06 (m, 4H), 2.07-1.90 (m, 2H), 1.87-1.63 (m, 7H), 1.63-1.49 (m, 2H), 1.48-1.33 (m, 4H), 1.31-1.10 (m, 6H), 0.98-0.89 (m, 1H), 0.78 (t, J=7.5 Hz, 2H). LCMS: C₄₅H₆₁N₇O₅ requires: 779.5, found: m/z=780.2 [M+H]⁺.

### Example 128

### (R)—N-(1-(2-(1-(4-(2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-N—ISOPROPOXY-4-(METHOXYMETHYL)PIPERIDINE-4-CARBOXAMIDE

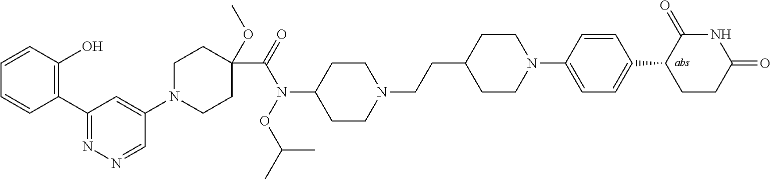 [SMILES: COC1(C(=O)N(OC(C)C)C2CCN(CCC3CCN(c4ccc([C@H]5CCC(=O)NC5=O)cc4)CC3)CC2)CCN(c2cnnc(-c3ccccc3O)c2)CC1]

### Step 1: Synthesis of benzyl 4-((1-(tert-butoxycarbonyl)piperidin-4-yl)(isopropoxy)carbamoyl)-4-(methoxymethyl)piperidine-1-carboxylate

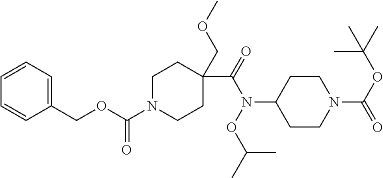 [SMILES: COCC1(C(=O)N(OC(C)C)C2CCN(C(=O)OC(C)(C)C)CC2)CCN(C(=O)OCc2ccccc2)CC1]

To a solution of 1-[(benzyloxy)carbonyl]-4-(methoxymethyl)piperidine-4-carboxylic acid (400.00 mg, 1.30 mmol) and tert-butyl 4-(isopropoxyamino)piperidine-1-carboxylate (420.31 mg, 1.63 mmol) in CH₂Cl₂ (3.00 mL) was added N,N-diisopropylethylamine (909.25 μL, 0.67 g, 5.21 mmol). Then, bis(2-oxo-1,3-oxazolidin-3-yl)phosphinoyl chloride (993.90 mg, 3.90 mmol) was added and the reaction mixture was stirred at 40° C. until complete by LCMS. Then, the reaction was filtered and the solvent removed under reduced pressure. The crude product was purified on silica gel with a gradient of EtOAc/Hex 0-100% to afford the desired product as a colorless oil. LCMS: C₂₉H₄₅N₃O₇ requires 547.3, found: m/z=448.2 [M-BOC]⁺.

### Step 2: Synthesis of tert-butyl 4-(N-isopropoxy-4-(methoxymethyl)piperidine-4-carboxamido)piperidine-1-carboxylate

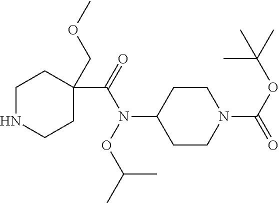 [SMILES: COCC1(C(=O)N(OC(C)C)C2CCN(C(=O)OC(C)(C)C)CC2)CCNCC1]

To a round bottom flask containing palladium (194.31 mg, 0.18 mmol) and EtOH (1.50 mL) was added benzyl 4-{[1-(tert-butoxycarbonyl)piperidin-4-yl](isopropoxy)carbamoyl}-4-(methoxymethyl)piperidine-1-carboxylate (200.00 mg, 0.36 mmol) dissolved in THF (1.5 mL). The flask was fitted with a three-way adapter and a hydrogen balloon. The atmosphere was changed to hydrogen, and the reaction was stirred until complete by LCMS. Then, the crude product was filtered through a plug of celite, and the solvent was removed under reduced pressure to afford the desired product as a dark brown oil (130 mg, 86%). LCMS: C₂₁H₃₉N₃O₅ requires 413.3, found: m/z=414.4 [M+H]⁺.

### Step 3: Synthesis of tert-butyl 4-(N-isopropoxy-1-(6-(2-(methoxymethoxy)phenyl)pyridazin-4-yl)-4-(methoxymethyl)piperidine-4-carboxamido)piperidine-1-carboxylate

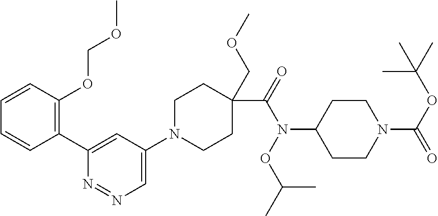 [SMILES: COCOc1ccccc1-c1cc(N2CCC(COC)(C(=O)N(OC(C)C)C3CCN(C(=O)OC(C)(C)C)CC3)CC2)cnn1]

A mixture of tert-butyl 4-[N-isopropoxy-4-(methoxymethyl)piperidine-4-amido]piperidine-1-carboxylate (130.00 mg, 0.31 mmol), 5-chloro-3-[2-(methoxymethoxy)phenyl]pyridazine (78.80 mg, 0.31 mmol) and N,N-diisopropylethylamine (274.51 μL, 0.20 g, 1.57 mmol) was dissolved in DMSO (1.20 mL). Then, the flask was heated to 100° C. and allowed to react until complete by LCMS. Then, it was quenched with water, extracted with ethyl acetate, washed with brine, dried over Na₂SO₄, filtered and the solvent removed under reduced pressure. The crude oil was purified via flash chromatography in silica with a gradient of 0-20% MeOH/CH₂Cl₂ to afford the desired product as a yellow oil. LCMS: C₃₃H₄₉N₅O₇ requires 627.4, found: m/z=628.4 [M+H]⁺.

### Step 4: Synthesis of 1-(6-(2-hydroxyphenyl)pyridazin-4-yl)-N-isopropoxy-4-(methoxymethyl)-N-(piperidin-4-yl)piperidine-4-carboxamide

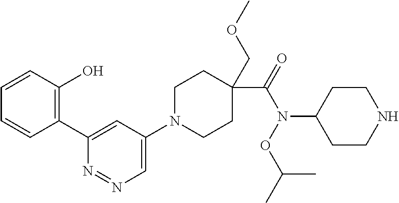 [SMILES: COCC1(C(=O)N(OC(C)C)C2CCNCC2)CCN(c2cnnc(-c3ccccc3O)c2)CC1]

tert-butyl 4-(N-isopropoxy-1-(6-(2-(methoxymethoxy)phenyl)pyridazin-4-yl)-4-(methoxymethyl)piperidine-4-carboxamido)piperidine-1-carboxylate was dissolved in 2.00 mL of THF and 4M HCl in Dioxane (1.00 mL) was added in one portion. The reaction was stirred at room temperature until completed by LCMS and then concentrated in vacuo to yield the desired product as a yellow solid. LCMS: C₂₆H₃₇N₅O₄ requires: 483.3, found: m/z=484.4 [M+H]⁺.

### Step 5: Synthesis of the Title Compound

The title compound was prepared as described for Example 1, Step 2, using 1-(6-(2-hydroxyphenyl)pyridazin-4-yl)-N-isopropoxy-4-(methoxymethyl)-N-(piperidin-4-yl)piperidine-4-carboxamide and (R)-2-(1-(4-(2,6-dioxopiperidin-3-yl)phenyl)piperidin-4-yl)acetaldehyde to afford the title compound (7.2 mg, 18%). LCMS: C₄₄H₅₉N₇O₆ requires 781.5, found: m/z=782.4 [M+H]⁺.

### Example 129

### N-{1-[2-(1-{4-[(3R)-2,6-DIOXOPIPERIDIN-3-YL]PHENYL}PIPERIDIN-4-YL)ETHYL]PIPERIDIN-4-YL}-1-[6-(2-HYDROXYPHENYL)PYRIDAZIN-4-YL]-N,4-DIMETHYLPIPERIDINE-4-CARBOXAMIDE

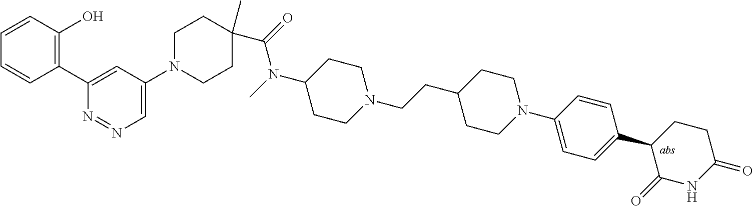 [SMILES: CN(C(=O)C1(C)CCN(c2cnnc(-c3ccccc3O)c2)CC1)C1CCN(CCC2CCN(c3ccc([C@H]4CCC(=O)NC4=O)cc3)CC2)CC1]

### Step 1: tert-butyl 4-(N-methyl-1-{6-[2-(methoxymethoxy)phenyl]pyridazin-4-yl}-4-methylpiperidine-4-amido)piperidine-1-carboxylate

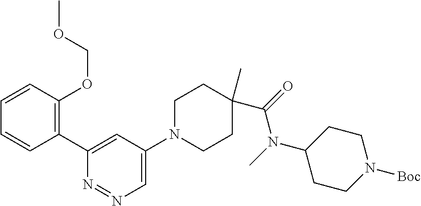 [SMILES: COCOc1ccccc1-c1cc(N2CCC(C)(C(=O)N(C)C3CCN(C(=O)OC(C)(C)C)CC3)CC2)cnn1]

To a solution of 1-{6-[2-(methoxymethoxy)phenyl]pyridazin-4-yl}-4-methylpiperidine-4-carboxylic acid (50.00 mg, 0.1399 mmol) in DMA (0.30 mL) was added tert-butyl 4-(methylamino)piperidine-1-carboxylate (44.97 mg, 0.2098 mmol) and N,N-diisopropylethylamine (73.10 μL, 0.05 g, 0.4197 mmol). [(dimethylamino)({[1,2,3]triazolo[4,5-b]pyridin-3-yloxy})methylidene]dimethylazanium; hexafluoro-lambda5-phosphanuide (63.83 mg, 0.1679 mmol) was added and the reaction was stirred at rt for 2 days. More amine (255 mg, 2 eq), DIPEA (0.276 mL, 2 eq) and HATU (226 mg, 2 eq) were added and after stirring overnight, the reaction was partitioned between DCM and water, washed DCM layer with sat NH4Cl and passed through a phase separator. The solvent was removed by rotovap to yield crude tert-butyl 4-(N-methyl-1-{6-[2-(methoxymethoxy)phenyl]pyridazin-4-yl}-4-methylpiperidine-4-amido)piperidine-1-carboxylate. LCMS: C₃₀H₄₃N₅O₅ requires: 553.4, found: m/z=554.4 [M+H]⁺.

### Step 2: 1-[6-(2-hydroxyphenyl)pyridazin-4-yl]-N,4-dimethyl-N-(piperidin-4-yl)piperidine-4-carboxamide

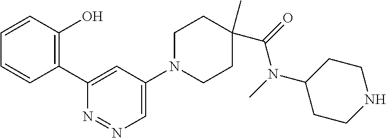 [SMILES: CN(C(=O)C1(C)CCN(c2cnnc(-c3ccccc3O)c2)CC1)C1CCNCC1]

1-[6-(2-hydroxyphenyl)pyridazin-4-yl]-N,4-dimethyl-N-(piperidin-4-yl)piperidine-4-carboxamide was prepared as described for Example 107 step 2. LCMS: C₂₃H₃₁N₅O₂ requires: 409.2, found: m/z=410.2 [M+H]⁺.

### Step 3: Synthesis of the Title Compound

The title compound was synthesized as described for Example 107 step 3, using 2-(1-{4-[(3R)-2,6-dioxopiperidin-3-yl]phenyl}piperidin-4-yl)acetaldehyde to yield N-{1-[2-(1-{4-[(3R)-2,6-dioxopiperidin-3-yl]phenyl}piperidin-4-yl)ethyl]piperidin-4-yl}-1-[6-(2-hydroxyphenyl)pyridazin-4-yl]-N,4-dimethylpiperidine-4-carboxamide (0.0064 g, 20.42%). LCMS: C₄₁H₅₃N₇O₄ requires: 707.4, found: m/z=708.4 [M+H]⁺.

### Example 130

### N-{1-[2-(1-{4-[(3R)-2,6-DIOXOPIPERIDIN-3-YL]PHENYL}PIPERIDIN-4-YL)ETHYL]PIPERIDIN-4-YL}-1-[6-(2-HYDROXYPHENYL)PYRIDAZIN-4-YL]-N-METHYL-4-PROPYLPIPERIDINE-4-CARBOXAMIDE

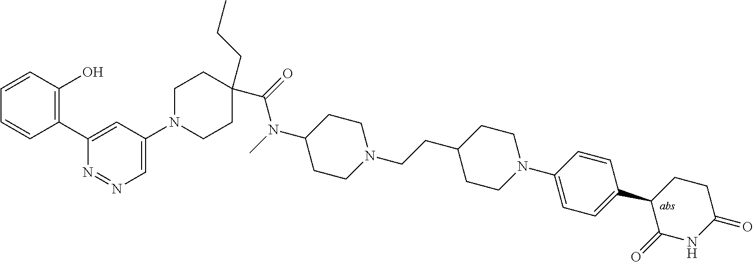 [SMILES: CCCC1(C(=O)N(C)C2CCN(CCC3CCN(c4ccc([C@H]5CCC(=O)NC5=O)cc4)CC3)CC2)CCN(c2cnnc(-c3ccccc3O)c2)CC1]

### Step 1: tert-butyl 4-(N-methyl-1-{6-[2-(methoxymethoxy)phenyl]pyridazin-4-yl}-4-propylpiperidine-4-amido)piperidine-1-carboxylate

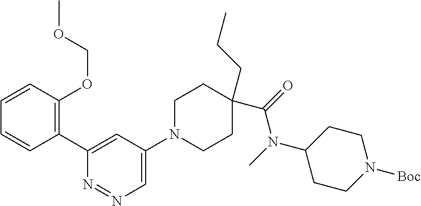 [SMILES: CCCC1(C(=O)N(C)C2CCN(C(=O)OC(C)(C)C)CC2)CCN(c2cnnc(-c3ccccc3OCOC)c2)CC1]

Tert-butyl 4-(N-methyl-1-{6-[2-(methoxymethoxy)phenyl]pyridazin-4-yl}-4-propylpiperidine-4-amido)piperidine-1-carboxylate was synthesized as described for Example 129, step 1 using 1-{6-[2-(methoxymethoxy)phenyl]pyridazin-4-yl}-4-propylpiperidine-4-carboxylic acid to yield tert-butyl 4-(N-methyl-1-{6-[2-(methoxymethoxy)phenyl]pyridazin-4-yl}-4-propylpiperidine-4-amido)piperidine-1-carboxylate (quantitative yield). LCMS: C₃₀H₄₃N₅O₅ requires: 581.4, found: m/z=582.4 [M+H]⁺.

### Step 2: 1-[6-(2-hydroxyphenyl)pyridazin-4-yl]-N-methyl-N-(piperidin-4-yl)-4-propylpiperidine-4-carboxamide

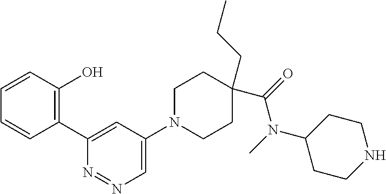 [SMILES: CCCC1(C(=O)N(C)C2CCNCC2)CCN(c2cnnc(-c3ccccc3O)c2)CC1]

1-[6-(2-hydroxyphenyl)pyridazin-4-yl]-N-methyl-N-(piperidin-4-yl)-4-propylpiperidine-4-carboxamide was prepared as described for Example 107 step 2. LCMS: C₂₅H₃₅N₅O₂ requires: 437.3, found: m/z=438.2 [M+H]⁺.

### Step 3: Synthesis of the Title Compound

The title compound was synthesized as described for Example 107 step 3, using 1-[6-(2-hydroxyphenyl)pyridazin-4-yl]-N-methyl-N-(piperidin-4-yl)-4-propylpiperidine-4-carboxamide hydrochloride and 2-(1-{4-[(3R)-2,6-dioxopiperidin-3-yl]phenyl}piperidin-4-yl)acetaldehyde to yield N-{1-[2-(1-{4-[(3R)-2,6-dioxopiperidin-3-yl]phenyl}piperidin-4-yl)ethyl]piperidin-4-yl}-1-[6-(2-hydroxyphenyl)pyridazin-4-yl]-N-methyl-4-propylpiperidine-4-carboxamide (0.0076 g, 21.28%). LCMS: C₄₃H₅₇N₇O₄ requires: 735.4, found: m/z=736.4 [M+H]⁺.

### Example 131

### (RS)—N-(1-(2-(1-(4-((R)-2,6-DIOXOPIPERIDIN-3-YL)PHENYL)PIPERIDIN-4-YL)ETHYL)PIPERIDIN-4-YL)-1-(6-(5-FLUORO-2-HYDROXYPHENYL)PYRIDAZIN-4-YL)-3-METHOXY-N-METHYLPYRROLIDINE-3-CARBOXAMIDE

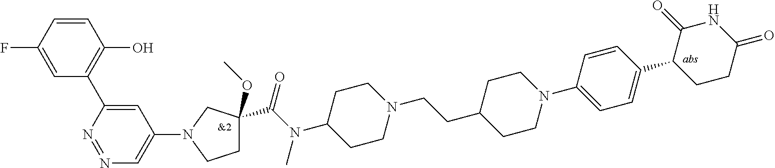 [SMILES: CO[C@@]1(C(=O)N(C)C2CCN(CCC3CCN(c4ccc([C@H]5CCC(=O)NC5=O)cc4)CC3)CC2)CCN(c2cnnc(-c3cc(F)ccc3O)c2)C1]

### Step 1: Synthesis of rac-(R)-1-(6-(5-fluoro-2-hydroxyphenyl)pyridazin-4-yl)-3-methoxy-N-methyl-N-(piperidin-4-yl)pyrrolidine-3-carboxamide hydrochloride

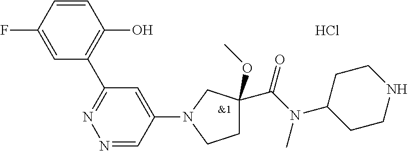 [SMILES: CO[C@@]1(C(=O)N(C)C2CCNCC2)CCN(c2cnnc(-c3cc(F)ccc3O)c2)C1 ; Cl]

The title compound was prepared as described for the synthesis of Example 1, Step 1, using rac-(R)-1-(6-(5-fluoro-2-(methoxymethoxy)phenyl)pyridazin-4-yl)-3-methoxypyrrolidine-3-carboxylic acid (Intermediate 27) and tert-butyl 4-(methylamino)piperidine-1-carboxylate to afford the title compound as a yellow oil. LCMS C₂₂H₂₈FN₅O₃ requires 429.2, found m/z=430.2 [M+H]⁺.

### Step 2: Synthesis of the Title Compound

The title compound was prepared as described for the synthesis of Example 2, Step 2, using rac-(R)-1-(6-(5-fluoro-2-hydroxyphenyl)pyridazin-4-yl)-3-methoxy-N-methyl-N-(piperidin-4-yl)pyrrolidine-3-carboxamide hydrochloride, to afford the title compound (18.3 mgs, 40% yield) as a white solid. LCMS: C₄₀H₅₀FN₇O₅ requires: 727.4, found: m/z=728.4 [M+H]⁺.

### Example 132

### N-{1-[2-(1-{4-[(3R)-2,6-DIOXOPIPERIDIN-3-YL]PHENYL}PIPERIDIN-4-YL)ETHYL]PIPERIDIN-4-YL}-N-ETHYL-1-[6-(2-HYDROXYPHENYL)PYRIDAZIN-4-YL]-4-METHYLPIPERIDINE-4-CARBOXAMIDE

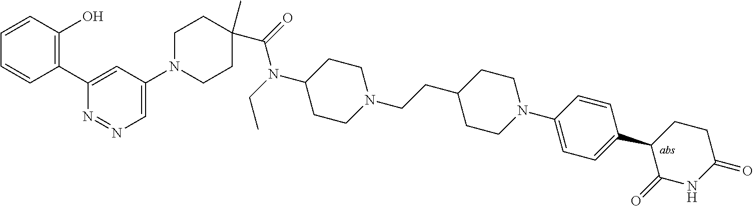 [SMILES: CCN(C(=O)C1(C)CCN(c2cnnc(-c3ccccc3O)c2)CC1)C1CCN(CCC2CCN(c3ccc([C@H]4CCC(=O)NC4=O)cc3)CC2)CC1]

### Step 1: benzyl 4-{[1-(tert-butoxycarbonyl)piperidin-4-yl](ethyl)carbamoyl}-4-methylpiperidine-1-carboxylate

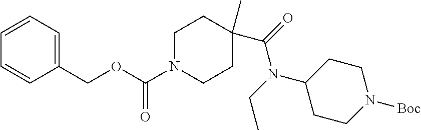 [SMILES: CCN(C(=O)C1(C)CCN(C(=O)OCc2ccccc2)CC1)C1CCN(C(=O)OC(C)(C)C)CC1]

To a solution of 1-[(benzyloxy)carbonyl]-4-methylpiperidine-4-carboxylic acid (222.00 mg, 0.8005 mmol) in DMA (2.00 mL) was added tert-butyl 4-(ethylamino)piperidine-1-carboxylate (219.34 mg, 0.9606 mmol) and N,N-diisopropylethylamine (327.68 μL, 0.24 g, 1.8812 mmol). Added bis(2-oxo-1,3-oxazolidin-3-yl)phosphinoyl chloride (244.54 mg, 0.9606 mmol) at rt and then placed the reaction in a 40° C. heating block. After 1 h, the reaction was purified directly by RP-HPLC to yield benzyl 4-{[1-(tert-butoxycarbonyl)piperidin-4-yl](ethyl)carbamoyl}-4-methylpiperidine-1-carboxylate (0.074 g, 18.96%). LCMS: C₂₇H₄₁N₃O₅ requires: 487.3, found: m/z=510.2 [M+Na]⁺.

### Step 2: tert-butyl 4-(N-ethyl-4-methylpiperidine-4-amido)piperidine-1-carboxylate

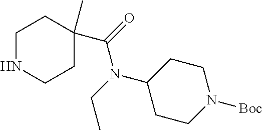 [SMILES: CCN(C(=O)C1(C)CCNCC1)C1CCN(C(=O)OC(C)(C)C)CC1]

Benzyl 4-{[1-(tert-butoxycarbonyl)piperidin-4-yl](ethyl)carbamoyl}-4-methylpiperidine-1-carboxylate (74.00 mg, 0.1518 mmol) was dissolved in ethanol (2.00 mL) and purged with nitrogen. Pd/C was added and the reaction was stirred under a hydrogen atmosphere (balloon). After a few hours, the reaction was filtered and concentrated down to an oil to yield tert-butyl 4-(N-ethyl-4-methylpiperidine-4-amido)piperidine-1-carboxylate (0.053 g, 98.80%). LCMS: C₁₉H₃₅N₃O₃ requires: 353.3, found: m/z=354.2 [M+H]⁺.

### Step 3: tert-butyl 4-[N-ethyl-1-(6-chloropyridazin-4-yl)-4-methylpiperidine-4-amido]piperidine-1-carboxylate

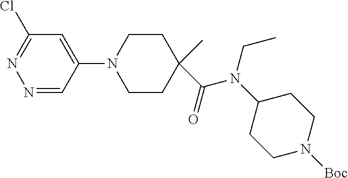 [SMILES: CCN(C(=O)C1(C)CCN(c2cnnc(Cl)c2)CC1)C1CCN(C(=O)OC(C)(C)C)CC1]

Tert-butyl 4-(N-ethyl-4-methylpiperidine-4-amino)piperidine-1-carboxylate (54.58 mg, 0.1544 mmol), 3,5-dichloropyridazine (23.00 mg, 0.1544 mmol) and N-methylmorpholine (37.34 μL, 0.03 g, 0.3397 mmol) were dissolved in DMSO (0.30 mL) and stirred in an 80° C. heating block for 30 min. After cooling, the reaction was diluted with 3 mL water and the product extracted with EtOAc (3×2 mL). The combined organic layers were dried with a phase separator and concentrated by rotary evaporation to yield tert-butyl 4-[N-ethyl-1-(6-chloropyridazin-4-yl)-4-methylpiperidine-4-amido]piperidine-1-carboxylate (0.0487 g, 67.69%). LCMS: C₂₃H₃₆ClN₅O₃ requires: 465.3, found: m/z=466.2 [M+H]⁺.

### Step 4: tert-butyl 4-{N-ethyl-1-[6-(2-hydroxyphenyl)pyridazin-4-yl]-4-methylpiperidine-4-amido}piperidine-1-carboxylate

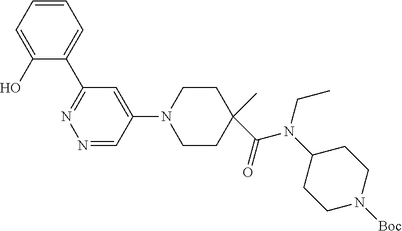 [SMILES: CCN(C(=O)C1(C)CCN(c2cnnc(-c3ccccc3O)c2)CC1)C1CCN(C(=O)OC(C)(C)C)CC1]

Tert-butyl 4-[N-ethyl-1-(6-chloropyridazin-4-yl)-4-methylpiperidine-4-amido]piperidine-1-carboxylate (48.70 mg, 0.1045 mmol) was dissolved in 1,4-dioxane (2.00 mL) and water (0.30 mL) along with 2-hydroxyphenylboronic acid (28.83 mg, 0.2090 mmol) and potassium carbonate (43.33 mg, 0.3135 mmol) and Pd(dppf)Cl2 (7.65 mg, 0.0105 mmol). The reaction was sealed under nitrogen and stirred in a 90° C. heating block. After 90 min, added more catalyst (15 mg) and continued heating for 60 min, then left at rt overnight. Added more boronic acid (15 mg, 1 eq), K-carb (14 mg, 1 eq) and catalyst (7.7 mg, 0.1 eq). Heated for another 90 min. Purified by RP-HPLC to yield tert-butyl 4-{N-ethyl-1-[6-(2-hydroxyphenyl)pyridazin-4-yl]-4-methylpiperidine-4-amido}piperidine-1-carboxylate (0.041 g, 74.92%). LCMS: C₂₉H₄₁N₅O₄ requires: 523.3, found: m/z=524.3 [M+H]⁺.

### Step 5: N-ethyl-1-[6-(2-hydroxyphenyl)pyridazin-4-yl]-4-methyl-N-(piperidin-4-yl)piperidine-4-carboxamide

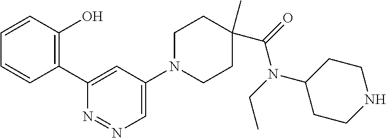 [SMILES: CCN(C(=O)C1(C)CCN(c2cnnc(-c3ccccc3O)c2)CC1)C1CCNCC1]

N-ethyl-1-[6-(2-hydroxyphenyl)pyridazin-4-yl]-4-methyl-N-(piperidin-4-yl)piperidine-4-carboxamide was prepared as described for Example 107 step 2. LCMS: C₂₄H₃₃N₅O₂ requires: 423.3, found: m/z=424.2 [M+H]⁺.

### Step 6: Synthesis of the Title Compound

The title compound was synthesized as described for Example 109 step 3, using N-ethyl-1-[6-(2-hydroxyphenyl)pyridazin-4-yl]-4-methyl-N-(piperidin-4-yl)piperidine-4-carboxamide hydrochloride and 2-(1-{4-[(3R)-2,6-dioxopiperidin-3-yl]phenyl}piperidin-4-yl)acetaldehyde to yield N-{1-[2-(1-{4-[(3R)-2,6-dioxopiperidin-3-yl]phenyl}piperidin-4-yl)ethyl]piperidin-4-yl}-N-ethyl-1-[6-(2-hydroxyphenyl)pyridazin-4-yl]-4-methylpiperidine-4-carboxamide (0.011 g, 43.03%). LCMS: C₄₂H₅₅N₇O₄ requires: 721.4, found: m/z=722.3 [M+H]⁺.

### Example 133

### 4-CYCLOPROPYL-N-{1-[2-(1-{4-[(3R)-2,6-DIOXOPIPERIDIN-3-YL]PHENYL}PIPERIDIN-4-YL)ETHYL]PIPERIDIN-4-YL}-1-[6-(2-HYDROXYPHENYL)PYRIDAZIN-4-YL]-N-METHYLPIPERIDINE-4-CARBOXAMIDE

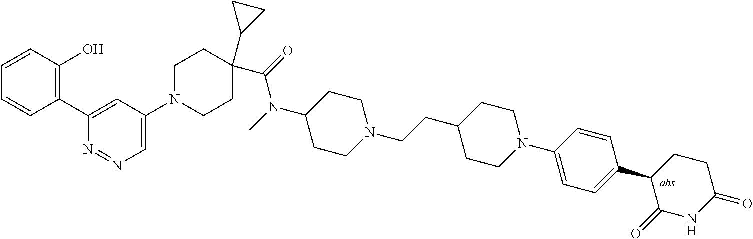 [SMILES: CN(C(=O)C1(C2CC2)CCN(c2cnnc(-c3ccccc3O)c2)CC1)C1CCN(CCC2CCN(c3ccc([C@H]4CCC(=O)NC4=O)cc3)CC2)CC1]

### Step 1: tert-butyl 4-(N-methyl-4-cyclopropyl-1-{6-[2-(methoxymethoxy)phenyl]pyridazin-4-yl}piperidine-4-amido)piperidine-1-carboxylate

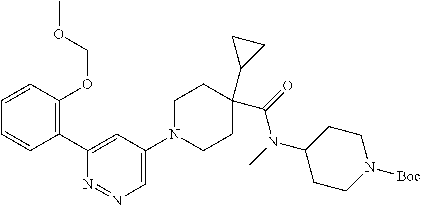 [SMILES: COCOc1ccccc1-c1cc(N2CCC(C(=O)N(C)C3CCN(C(=O)OC(C)(C)C)CC3)(C3CC3)CC2)cnn1]

Tert-butyl 4-(N-methyl-4-cyclopropyl-1-{6-[2-(methoxymethoxy)phenyl]pyridazin-4-yl}piperidine-4-amido)piperidine-1-carboxylate was synthesized as described for Example 129 step 1, using 4-cyclopropyl-1-{6-[2-(methoxymethoxy)phenyl]pyridazin-4-yl}piperidine-4-carboxylic acid and tert-butyl 4-(methylamino)piperidine-1-carboxylate to yield tert-butyl 4-(N-methyl-4-cyclopropyl-1-{6-[2-(methoxymethoxy)phenyl]pyridazin-4-yl}piperidine-4-amido)piperidine-1-carboxylate (0.086 g, 111.53%). LCMS: C₃₂H₄₅N₅O₅ requires: 579.3, found: m/z=580.4 [M+H]⁺.

### Step 2: 4-cyclopropyl-1-[6-(2-hydroxyphenyl)pyridazin-4-yl]-N-methyl-N-(piperidin-4-yl)piperidine-4-carboxamide

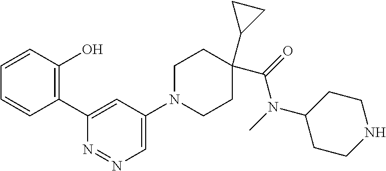 [SMILES: CN(C(=O)C1(C2CC2)CCN(c2cnnc(-c3ccccc3O)c2)CC1)C1CCNCC1]

4-cyclopropyl-1-[6-(2-hydroxyphenyl)pyridazin-4-yl]-N-methyl-N-(piperidin-4-yl)piperidine-4-carboxamide was prepared as described for Example 107 step 2. LCMS: C₂₅H₃₃N₅O₂ requires: 435.3, found: m/z=436.2 [M+H]⁺.

### Step 3: Synthesis of the Title Compound

The title compound was synthesized as described for Example 109 step 3, using 4-cyclopropyl-1-[6-(2-hydroxyphenyl)pyridazin-4-yl]-N-methyl-N-(piperidin-4-yl)piperidine-4-carboxamide hydrochloride and 2-(1-{4-[(3R)-2,6-dioxopiperidin-3-yl]phenyl}piperidin-4-yl)acetaldehyde to yield 4-cyclopropyl-N-{1-[2-(1-{4-[(3R)-2,6-dioxopiperidin-3-yl]phenyl}piperidin-4-yl)ethyl]piperidin-4-yl}-1-[6-(2-hydroxyphenyl)pyridazin-4-yl]-N-methylpiperidine-4-carboxamide (0.014 g, 60.52%). LCMS: C₄₃H₅₅N₇O₄ requires: 733.4, found: m/z=734.2 [M+H]⁺.

### Example 134

### RAC-N-{1-[2-(1-{4-[(3R)-2,6-DIOXOPIPERIDIN-3-YL]PHENYL}-4-FLUOROPIPERIDIN-4-YL)ETHYL]PIPERIDIN-4-YL}-4-ETHYL-1-[6-(2-HYDROXYPHENYL)PYRIDAZIN-4-YL]-N-METHYLPIPERIDINE-4-CARBOXAMIDE

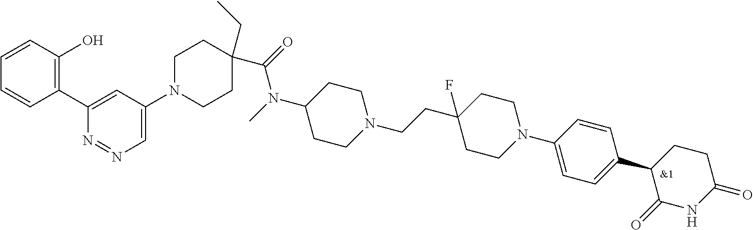 [SMILES: CCC1(C(=O)N(C)C2CCN(CCC3(F)CCN(c4ccc([C@H]5CCC(=O)NC5=O)cc4)CC3)CC2)CCN(c2cnnc(-c3ccccc3O)c2)CC1]

### Step 1: tert-butyl 4-(N-methyl-4-ethyl-1-{6-[2-(methoxymethoxy)phenyl]pyridazin-4-yl}piperidine-4-amido)piperidine-1-carboxylate

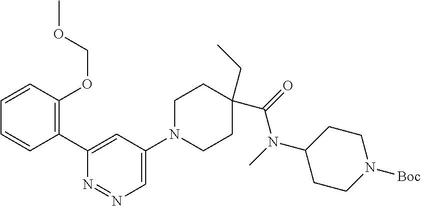 [SMILES: CCC1(C(=O)N(C)C2CCN(C(=O)OC(C)(C)C)CC2)CCN(c2cnnc(-c3ccccc3OCOC)c2)CC1]

Tert-butyl4-(N-methyl-4-ethyl-1-{6-[2-(methoxymethoxy)phenyl]pyridazin-4-yl}piperidine-4-amido)piperidine-1-carboxylate was synthesized as described for Example 129 step 1, using 4-ethyl-1-{6-[2-(methoxymethoxy)phenyl]pyridazin-4-yl}piperidine-4-carboxylic acid and tert-butyl 4-(methylamino)piperidine-1-carboxylate to yield tert-butyl 4-(N-methyl-4-ethyl-1-{6-[2-(methoxymethoxy)phenyl]pyridazin-4-yl}piperidine-4-amido)piperidine-1-carboxylate (quantitative yield). LCMS: C₃₁H₄₅N₅O₅ requires: 567.3, found: m/z=568.4 [M+H]⁺.

### Step 2: 4-ethyl-1-[6-(2-hydroxyphenyl)pyridazin-4-yl]-N-methyl-N-(piperidin-4-yl)piperidine-4-carboxamide

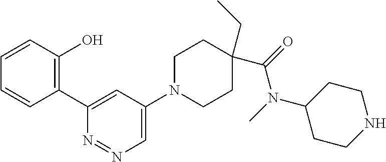 [SMILES: CCC1(C(=O)N(C)C2CCNCC2)CCN(c2cnnc(-c3ccccc3O)c2)CC1]

4-ethyl-1-[6-(2-hydroxyphenyl)pyridazin-4-yl]-N-methyl-N-(piperidin-4-yl)piperidine-4-carboxamide was prepared as described for Example 107 step 2. LCMS: C₂₄H₃₃N₅O₂ requires: 423.3, found: m/z=424.2 [M+H]⁺.

### Step 3: Synthesis of the Title Compound

Combined 4-ethyl-1-[6-(2-hydroxyphenyl)pyridazin-4-yl]-N-methyl-N-(piperidin-4-yl)piperidine-4-carboxamide hydrochloride (15.50 mg, 0.0337 mmol) and rac-2-(1-{4-[(3R)-2,6-dioxopiperidin-3-yl]phenyl}-4-fluoropiperidin-4-yl)acetaldehyde (12.32 mg, 0.0371 mmol) in DMA (0.30 mL) and then added sodium triacetoxyborohydride (10.71 mg, 0.0505 mmol) and stirred for 2 h, then purified directly by HTP to yield rac-N-{1-[2-(1-{4-[(3R)-2,6-dioxopiperidin-3-yl]phenyl}-4-fluoropiperidin-4-yl)ethyl]piperidin-4-yl}-4-ethyl-1-[6-(2-hydroxyphenyl)pyridazin-4-yl]-N-methylpiperidine-4-carboxamide (0.0034 g, 12.52%). LCMS: C₄₂H₅₄FN₇O₄ requires: 739.4, found: m/z=740.3 [M+H]⁺.

### Example 135

### RAC-N-{1-[2-(1-{4-[(3R)-2,6-DIOXOPIPERIDIN-3-YL]-3-FLUOROPHENYL}PIPERIDIN-4-YL)ETHYL]PIPERIDIN-4-YL}-1-[6-(2-HYDROXYPHENYL)PYRIDAZIN-4-YL]-N,4-DIMETHYLPIPERIDINE-4-CARBOXAMIDE

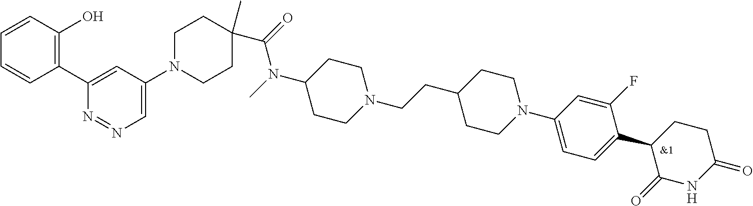 [SMILES: CN(C(=O)C1(C)CCN(c2cnnc(-c3ccccc3O)c2)CC1)C1CCN(CCC2CCN(c3ccc([C@H]4CCC(=O)NC4=O)c(F)c3)CC2)CC1]

The title compound was synthesized as described for Example 109 step 3, using 1-[6-(2-hydroxyphenyl)pyridazin-4-yl]-N,4-dimethyl-N-(piperidin-4-yl)piperidine-4-carboxamide hydrochloride and rac-2-(1-{4-[(3R)-2,6-dioxopiperidin-3-yl]-3-fluorophenyl}piperidin-4-yl)acetaldehyde to yield rac-N-{1-[2-(1-{4-[(3R)-2,6-dioxopiperidin-3-yl]-3-fluorophenyl}piperidin-4-yl)ethyl]piperidin-4-yl}-1-[6-(2-hydroxyphenyl)pyridazin-4-yl]-N,4-dimethylpiperidine-4-carboxamide (0.0068 g, 25.67%). LCMS: C₄₁H₅₂FN₇O₄ requires: 725.4, found: m/z=726.3 [M+H]⁺.

### Example 136

### RAC-N-{1-[2-(1-{4-[(3R)-2,6-DIOXOPIPERIDIN-3-YL]-3-FLUOROPHENYL}PIPERIDIN-4-YL)ETHYL]PIPERIDIN-4-YL}-4-ETHYL-1-[6-(2-HYDROXYPHENYL)PYRIDAZIN-4-YL]-N-METHYLPIPERIDINE-4-CARBOXAMIDE

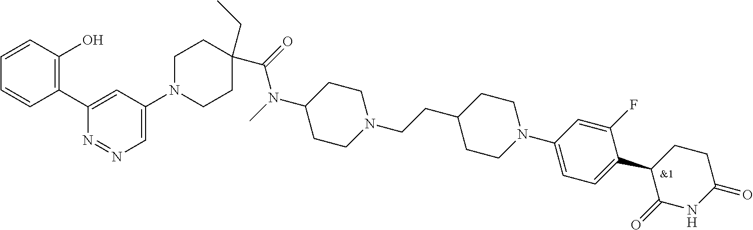 [SMILES: CCC1(C(=O)N(C)C2CCN(CCC3CCN(c4ccc([C@H]5CCC(=O)NC5=O)c(F)c4)CC3)CC2)CCN(c2cnnc(-c3ccccc3O)c2)CC1]

The title compound was synthesized as described for Example 109 step 3, using 4-ethyl-1-[6-(2-hydroxyphenyl)pyridazin-4-yl]-N-methyl-N-(piperidin-4-yl)piperidine-4-carboxamide hydrochloride and rac-2-(1-{4-[(3R)-2,6-dioxopiperidin-3-yl]-3-fluorophenyl}piperidin-4-yl)acetaldehyde to yield rac-N-{1-[2-(1-{4-[(3R)-2,6-dioxopiperidin-3-yl]-3-fluorophenyl}piperidin-4-yl)ethyl]piperidin-4-yl}-4-ethyl-1-[6-(2-hydroxyphenyl)pyridazin-4-yl]-N-methylpiperidine-4-carboxamide (0.0046 g, 17.37%). LCMS: C₄₂H₅₄FN₇O₄ requires: 739.4, found: m/z=740.3 [M+H]⁺.

### Example 137

### RAC-N-{1-[2-(1-{4-[(3R)-2,6-DIOXOPIPERIDIN-3-YL]-3-FLUOROPHENYL}PIPERIDIN-4-YL)ETHYL]PIPERIDIN-4-YL}-1-[6-(2-HYDROXYPHENYL)PYRIDAZIN-4-YL]-N-METHYL-4-PROPYLPIPERIDINE-4-CARBOXAMIDE

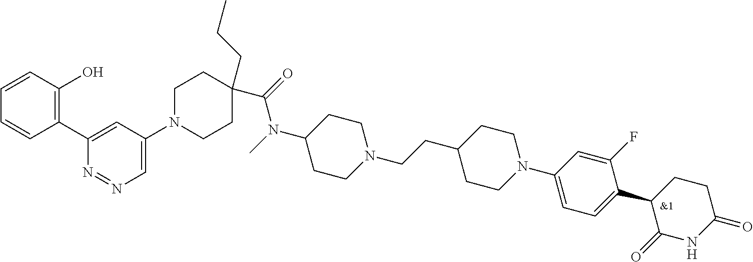 [SMILES: CCCC1(C(=O)N(C)C2CCN(CCC3CCN(c4ccc([C@H]5CCC(=O)NC5=O)c(F)c4)CC3)CC2)CCN(c2cnnc(-c3ccccc3O)c2)CC1]

The title compound was synthesized as described for Example 109 step 3, using 1-[6-(2-hydroxyphenyl)pyridazin-4-yl]-N-methyl-N-(piperidin-4-yl)-4-propylpiperidine-4-carboxamide hydrochloride and rac-2-(1-{4-[(3R)-2,6-dioxopiperidin-3-yl]-3-fluorophenyl}piperidin-4-yl)acetaldehyde to yield rac-N-{1-[2-(1-{4-[(3R)-2,6-dioxopiperidin-3-yl]-3-fluorophenyl}piperidin-4-yl)ethyl]piperidin-4-yl}-1-[6-(2-hydroxyphenyl)pyridazin-4-yl]-N-methyl-4-propylpiperidine-4-carboxamide (0.0053 g, 19.63%). LCMS: C₄₃H₅₆FN₇O₄ requires: 753.4, found: m/z=754.3 [M+H]⁺.

### Example 138

### RAC-N-{1-[2-(1-{4-[(3R)-2,6-DIOXOPIPERIDIN-3-YL]-3,5-DIFLUOROPHENYL}PIPERIDIN-4-YL)ETHYL]PIPERIDIN-4-YL}-4-ETHYL-1-[6-(2-HYDROXYPHENYL)PYRIDAZIN-4-YL]-N-METHYLPIPERIDINE-4-CARBOXAMIDE

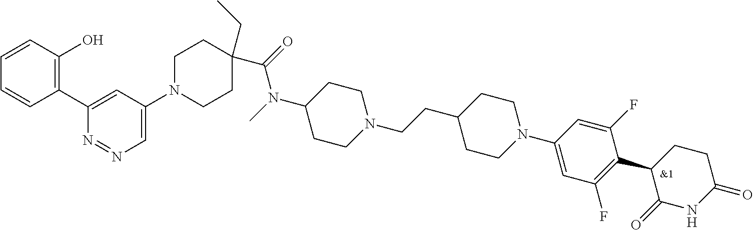 [SMILES: CCC1(C(=O)N(C)C2CCN(CCC3CCN(c4cc(F)c([C@H]5CCC(=O)NC5=O)c(F)c4)CC3)CC2)CCN(c2cnnc(-c3ccccc3O)c2)CC1]

The title compound was synthesized as described for Example 109 step 3, using 4-ethyl-1-[6-(2-hydroxyphenyl)pyridazin-4-yl]-N-methyl-N-(piperidin-4-yl)piperidine-4-carboxamide hydrochloride and rac-2-(1-{4-[(3R)-2,6-dioxopiperidin-3-yl]-3,5-difluorophenyl}piperidin-4-yl)acetaldehyde to yield rac-N-{1-[2-(1-{4-[(3R)-2,6-dioxopiperidin-3-yl]-3,5-difluorophenyl}piperidin-4-yl)ethyl]piperidin-4-yl}-4-ethyl-1-[6-(2-hydroxyphenyl)pyridazin-4-yl]-N-methylpiperidine-4-carboxamide (0.0042 g, 15.50%). LCMS: C₄₂H₅₃F₂N₇O₄ requires: 757.4, found: m/z=758.6 [M+H]⁺.

### Example 139

### RAC-N-{1-[2-(1-{4-[(3R)-2,6-DIOXOPIPERIDIN-3-YL]-3-FLUOROPHENYL}PIPERIDIN-4-YL)ETHYL]PIPERIDIN-4-YL}-1-[6-(2-HYDROXYPHENYL)PYRIDAZIN-4-YL]-N-(2-METHOXYETHYL)-4-METHYLPIPERIDINE-4-CARBOXAMIDE

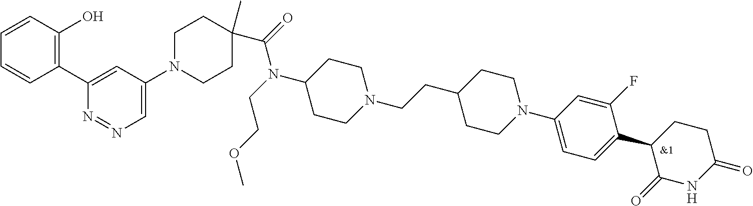 [SMILES: COCCN(C(=O)C1(C)CCN(c2cnnc(-c3ccccc3O)c2)CC1)C1CCN(CCC2CCN(c3ccc([C@H]4CCC(=O)NC4=O)c(F)c3)CC2)CC1]

### Step 1: benzyl 4-{[1-(tert-butoxycarbonyl)piperidin-4-yl](2-methoxyethyl)carbamoyl}-4-methylpiperidine-1-carboxylate

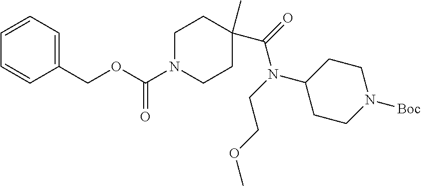 [SMILES: COCCN(C(=O)C1(C)CCN(C(=O)OCc2ccccc2)CC1)C1CCN(C(=O)OC(C)(C)C)CC1]

Benzyl 4-{[1-(tert-butoxycarbonyl)piperidin-4-yl](2-methoxyethyl)carbamoyl}-4-methylpiperidine-1-carboxylate was synthesized as described for Example 132 step 1 using 1-[(benzyloxy)carbonyl]-4-methylpiperidine-4-carboxylic acid and tert-butyl 4-[(2-methoxyethyl)amino]piperidine-1-carboxylate to yield benzyl 4-{[1-(tert-butoxycarbonyl)piperidin-4-yl](2-methoxyethyl)carbamoyl}-4-methylpiperidine-1-carboxylate (0.138 g, 18.76%). LCMS: C₂₈H₄₃N₃O₆ requires: 517.3, found: m/z=540.4 [M+Na]⁺.

### Step 2: tert-butyl 4-[N-(2-methoxyethyl)-4-methylpiperidine-4-amido]piperidine-1-carboxylate

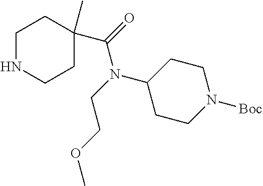 [SMILES: COCCN(C(=O)C1(C)CCNCC1)C1CCN(C(=O)OC(C)(C)C)CC1]

Tert-butyl 4-[N-(2-methoxyethyl)-4-methylpiperidine-4-amido]piperidine-1-carboxylate was synthesized as described for Example 132 step 2 (quantitative yield). LCMS: C₂₀H₃₇N₃O₄ requires: 383.3, found: m/z=384.2 [M+H]⁺.

### Step 3: tert-butyl 4-[N-(2-methoxyethyl)-1-[6-(2-hydroxyphenyl)pyridazin-4-yl]-4-methylpiperidine-4-amido]piperidine-1-carboxylate

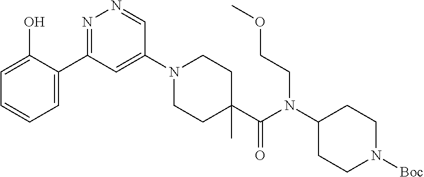 [SMILES: COCCN(C(=O)C1(C)CCN(c2cnnc(-c3ccccc3O)c2)CC1)C1CCN(C(=O)OC(C)(C)C)CC1]

Tert-butyl 4-[N-(2-methoxyethyl)-4-methylpiperidine-4-amido]piperidine-1-carboxylate (93.55 mg, 0.2439 mmol), 2-(5-chloropyridazin-3-yl)phenol (42.00 mg, 0.2033 mmol) and N,N-diisopropylethylamine (141.62 μL, 0.11 g, 0.8130 mmol) were dissolved in DMSO (1.00 mL) and stirred in an 100° C. heating block. After 3 h, the reaction was cooled and directly purified by RP-HPLC to yield tert-butyl 4-[N-(2-methoxyethyl)-1-[6-(2-hydroxyphenyl)pyridazin-4-yl]-4-methylpiperidine-4-amido]piperidine-1-carboxylate (0.108 g, 95.96%). LCMS: C₃₀H₄₃N₅O₅ requires: 553.3, found: m/z=554.4 [M+H]⁺.

### Step 4: 1-[6-(2-hydroxyphenyl)pyridazin-4-yl]-N-(2-methoxyethyl)-4-methyl-N-(piperidin-4-yl)piperidine-4-carboxamide

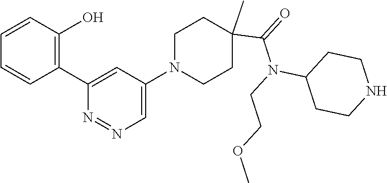 [SMILES: COCCN(C(=O)C1(C)CCN(c2cnnc(-c3ccccc3O)c2)CC1)C1CCNCC1]

1-[6-(2-hydroxyphenyl)pyridazin-4-yl]-N-(2-methoxyethyl)-4-methyl-N-(piperidin-4-yl)piperidine-4-carboxamide was prepared as described for Example 107 step 2. LCMS: C₂₅H₃₅N₅O₃ requires: 453.3, found: m/z=454.2 [M+H]⁺.

### Step 5: Synthesis of the Title Compound

Combined 1-[6-(2-hydroxyphenyl)pyridazin-4-yl]-N-(2-methoxyethyl)-4-methyl-N-(piperidin-4-yl)piperidine-4-carboxamide hydrochloride (15.00 mg, 0.0306 mmol) and rac-2-(1-{4-[(3R)-2,6-dioxopiperidin-3-yl]-3-fluorophenyl}piperidin-4-yl)acetaldehyde (11.19 mg, 0.0337 mmol) in DMA (0.30 mL) and then added sodium triacetoxyborohydride (9.73 mg, 0.0459 mmol) and stirred for 2 h, then purified directly by RP-HPLC to yield rac-N-{1-[2-(1-{4-[(3R)-2,6-dioxopiperidin-3-yl]-3-fluorophenyl}piperidin-4-yl)ethyl]piperidin-4-yl}-1-[6-(2-hydroxyphenyl)pyridazin-4-yl]-N-(2-methoxyethyl)-4-methylpiperidine-4-carboxamide (0.0079 g, 33.05%). LCMS: C₄₃H₅₆FN₇O₅ requires: 769.4, found: m/z=770.4 [M+H]⁺.

### Example 140

### N-CYCLOPROPYL-N-{1-[2-(1-{4-[(3R)-2,6-DIOXOPIPERIDIN-3-YL]PHENYL}PIPERIDIN-4-YL)ETHYL]PIPERIDIN-4-YL}-1-[6-(2-HYDROXYPHENYL)PYRIDAZIN-4-YL]-4-METHYLPIPERIDINE-4-CARBOXAMIDE

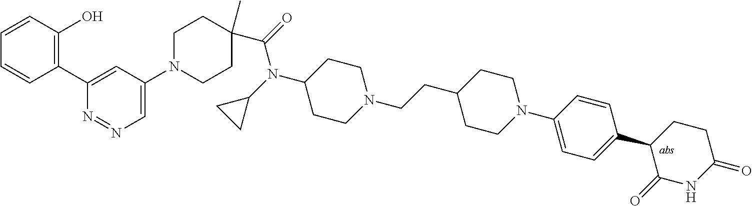 [SMILES: CC1(C(=O)N(C2CC2)C2CCN(CCC3CCN(c4ccc([C@H]5CCC(=O)NC5=O)cc4)CC3)CC2)CCN(c2cnnc(-c3ccccc3O)c2)CC1]

### Step 1: benzyl 4-{[1-(tert-butoxycarbonyl)piperidin-4-yl](cyclopropyl)carbamoyl}-4-methylpiperidine-1-carboxylate

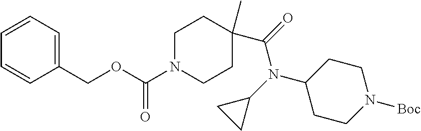 [SMILES: CC(C)(C)OC(=O)N1CCC(N(C(=O)C2(C)CCN(C(=O)OCc3ccccc3)CC2)C2CC2)CC1]

benzyl 4-{[1-(tert-butoxycarbonyl)piperidin-4-yl](cyclopropyl)carbamoyl}-4-methylpiperidine-1-carboxylate was synthesized as described for Example 132 step 1, using 1-[(benzyloxy)carbonyl]-4-methylpiperidine-4-carboxylic acid and tert-butyl 4-(cyclopropylamino)piperidine-1-carboxylate to yield benzyl 4-{[1-(tert-butoxycarbonyl)piperidin-4-yl](cyclopropyl)carbamoyl}-4-methylpiperidine-1-carboxylate (0.126 g, 18.02%). LCMS: C₂₈H₄₁N₃O₅ requires: 499.3, found: m/z=522.4 [M+Na]⁺.

### Step 2: tert-butyl 4-(N-cyclopropyl-4-methylpiperidine-4-amido)piperidine-1-carboxylate

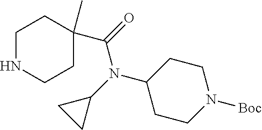 [SMILES: CC(C)(C)OC(=O)N1CCC(N(C(=O)C2(C)CCNCC2)C2CC2)CC1]

Tert-butyl 4-(N-cyclopropyl-4-methylpiperidine-4-amido)piperidine-1-carboxylate was synthesized as described for Example 132 step 2 (quantitative yield). LCMS: C₂₀H₃₅N₃O₃ requires: 365.3, found: m/z=366.4 [M+H]⁺.

### Step 3: tert-butyl 4-{N-cyclopropyl-1-[6-(2-hydroxyphenyl)pyridazin-4-yl]-4-methylpiperidine-4-amido}piperidine-1-carboxylate

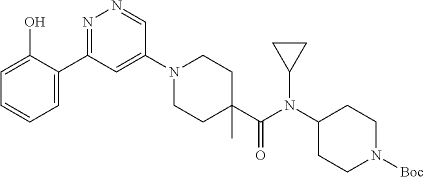 [SMILES: CC(C)(C)OC(=O)N1CCC(N(C(=O)C2(C)CCN(c3cnnc(-c4ccccc4O)c3)CC2)C2CC2)CC1]

tert-butyl 4-{N-cyclopropyl-1-[6-(2-hydroxyphenyl)pyridazin-4-yl]-4-methylpiperidine-4-amido}piperidine-1-carboxylate was synthesized as described for Example 139 step 3, using tert-butyl 4-(N-cyclopropyl-4-methylpiperidine-4-amido)piperidine-1-carboxylate to yield tert-butyl 4-{N-cyclopropyl-1-[6-(2-hydroxyphenyl)pyridazin-4-yl]-4-methylpiperidine-4-amido}piperidine-1-carboxylate (0.111 g, 101.94%). LCMS: C₃₀H₄₁N₅O₄ requires: 535.3, found: m/z=536.4 [M+H]⁺.

### Step 4: N-cyclopropyl-1-[6-(2-hydroxyphenyl)pyridazin-4-yl]-4-methyl-N-(piperidin-4-yl)piperidine-4-carboxamide

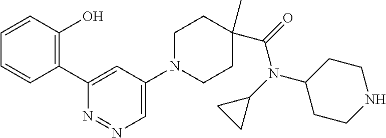 [SMILES: CC1(C(=O)N(C2CCNCC2)C2CC2)CCN(c2cnnc(-c3ccccc3O)c2)CC1]

N-cyclopropyl-1-[6-(2-hydroxyphenyl)pyridazin-4-yl]-4-methyl-N-(piperidin-4-yl)piperidine-4-carboxamide was prepared as described for Example 107 step 2. LCMS: C₂₅H₃₃N₅O₂ requires: 435.3, found: m/z=436.4 [M+H]⁺.

### Step 5: Synthesis of the Title Compound

The title compound was synthesized as described for Example 109 step 3, using N-cyclopropyl-1-[6-(2-hydroxyphenyl)pyridazin-4-yl]-4-methyl-N-(piperidin-4-yl)piperidine-4-carboxamide hydrochloride and 2-(1-{4-[(3R)-2,6-dioxopiperidin-3-yl]phenyl}piperidin-4-yl)acetaldehyde to yield N-cyclopropyl-N-{1-[2-(1-{4-[(3R)-2,6-dioxopiperidin-3-yl]phenyl}piperidin-4-yl)ethyl]piperidin-4-yl}-1-[6-(2-hydroxyphenyl)pyridazin-4-yl]-4-methylpiperidine-4-carboxamide (0.014 g, 59.99%). LCMS: C₄₃H₅₅N₇O₄ requires: 733.4, found: m/z=734.3 [M+H]⁺.

### Example 141

### RAC-N-CYCLOPROPYL-N-{1-[2-(1-{4-[(3R)-2,6-DIOXOPIPERIDIN-3-YL]PHENYL}-4-FLUOROPIPERIDIN-4-YL)ETHYL]PIPERIDIN-4-YL}-1-[6-(2-HYDROXYPHENYL)PYRIDAZIN-4-YL]-4-METHYLPIPERIDINE-4-CARBOXAMIDE

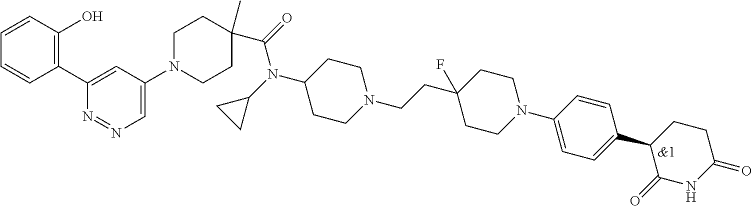 [SMILES: CC1(C(=O)N(C2CC2)C2CCN(CCC3(F)CCN(c4ccc([C@H]5CCC(=O)NC5=O)cc4)CC3)CC2)CCN(c2cnnc(-c3ccccc3O)c2)CC1]

The title compound was synthesized as described for Example 109 step 3, using N-cyclopropyl-1-[6-(2-hydroxyphenyl)pyridazin-4-yl]-4-methyl-N-(piperidin-4-yl)piperidine-4-carboxamide hydrochloride and rac-2-(1-{4-[(3R)-2,6-dioxopiperidin-3-yl]phenyl}-4-fluoropiperidin-4-yl)acetaldehyde to yield rac-N-cyclopropyl-N-{1-[2-(1-{4-[(3R)-2,6-dioxopiperidin-3-yl]phenyl}-4-fluoropiperidin-4-yl)ethyl]piperidin-4-yl}-1-[6-(2-hydroxyphenyl)pyridazin-4-yl]-4-methylpiperidine-4-carboxamide (0.0091 g, 35.49%). LCMS: C₄₃H₅₄FN₇O₄ requires: 751.4, found: m/z=752.3 [M+H]⁺.

### Example 142

### N-{1-[2-(1-{4-[(3R)-2,6-DIOXOPIPERIDIN-3-YL]PHENYL}PIPERIDIN-4-YL)ETHYL]PIPERIDIN-4-YL}-1-[6-(2-HYDROXYPHENYL)PYRIDAZIN-4-YL]-N-(2-METHOXYETHYL)-4-METHYLPIPERIDINE-4-CARBOXAMIDE

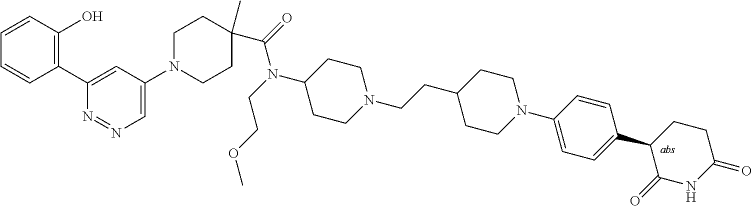 [SMILES: COCCN(C(=O)C1(C)CCN(c2cnnc(-c3ccccc3O)c2)CC1)C1CCN(CCC2CCN(c3ccc([C@H]4CCC(=O)NC4=O)cc3)CC2)CC1]

The title compound was synthesized as described for Example 109 step 3, using 1-[6-(2-hydroxyphenyl)pyridazin-4-yl]-N-(2-methoxyethyl)-4-methyl-N-(piperidin-4-yl)piperidine-4-carboxamide hydrochloride and 2-(1-{4-[(3R)-2,6-dioxopiperidin-3-yl]phenyl}piperidin-4-yl)acetaldehyde to yield N-{1-[2-(1-{4-[(3R)-2,6-dioxopiperidin-3-yl]phenyl}piperidin-4-yl)ethyl]piperidin-4-yl}-1-[6-(2-hydroxyphenyl)pyridazin-4-yl]-N-(2-methoxyethyl)-4-methylpiperidine-4-carboxamide (0.0039 g, 15.36%). LCMS: C₄₃H₅₇N₇O₅ requires: 751.4, found: m/z=752.3 [M+H]⁺.

### Example 143

### RAC-(1R,5S,8S)—N-(CYCLOPROPYLMETHYL)-N-(1-(2-(1-(4-((R)-2,6-DIOXOPIPERIDIN-3-YL)-3-FLUOROPHENYL)PIPERIDIN-4-YL)ETHYL)PIPERIDIN-4-YL)-3-(6-(2-HYDROXYPHENYL)PYRIDAZIN-4-YL)-3-AZABICYCLO[3.2.1]OCTANE-8-CARBOXAMIDE

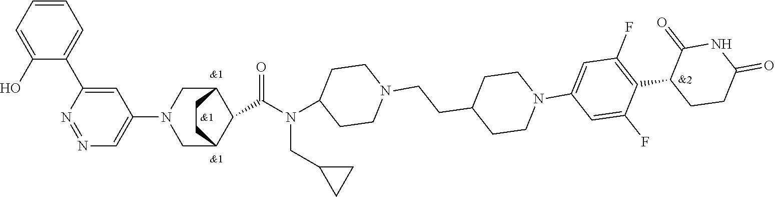 [SMILES: O=C1CC[C@H](c2c(F)cc(N3CCC(CCN4CCC(N(CC5CC5)C(=O)[C@H]5[C@@H]6CC[C@H]5CN(c5cnnc(-c7ccccc7O)c5)C6)CC4)CC3)cc2F)C(=O)N1]

### Step 1: Synthesis of the Title Compound

N-(cyclopropylmethyl)-3-[6-(2-hydroxyphenyl)pyridazin-4-yl]-N-(piperidin-4-yl)-3-azabicyclo[3.2.1]octane-8-carboxamide hydrochloride (8.00 mg, 0.0150 mmol) and 2-(1-{4-[(3R)-2,6-dioxopiperidin-3-yl]-3-fluorophenyl}piperidin-4-yl)acetaldehyde (5.18 mg, 0.0165 mmol) were added to a vial and DMA (0.50 mL) was added. Sodium triacetoxyborohydride (9.52 mg, 0.0449 mmol) was then added the reaction was stirred at RT for 30 mins and checked by LCMS. Once complete, the reaction was directly purified by RP—FC to afford the title compound (9.1 mg, 72.3%) as a formate salt. LCMS: C₄₅H₅₆FN₇O₄ requires: 777.4, found: m/z=778.3 [M+H]⁺.

### Example 144

### (RS)—N-(1-(2-(1-(4-((R)-2,6-DIOXOPIPERIDIN-3-YL)PHENYL)PIPERIDIN-4-YL)ETHYL)PIPERIDIN-4-YL)-3-FLUORO-1-(6-(2-HYDROXYPHENYL)PYRIDAZIN-4-YL)-N-METHYLPYRROLIDINE-3-CARBOXAMIDE

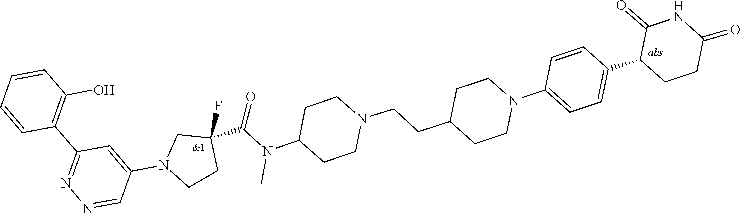 [SMILES: CN(C(=O)[C@]1(F)CCN(c2cnnc(-c3ccccc3O)c2)C1)C1CCN(CCC2CCN(c3ccc([C@H]4CCC(=O)NC4=O)cc3)CC2)CC1]

### Step 1: Synthesis of rac-(R)-3-fluoro-1-(6-(2-hydroxyphenyl)pyridazin-4-yl)-N-methyl-N-(piperidin-4-yl)pyrrolidine-3-carboxamide hydrochloride

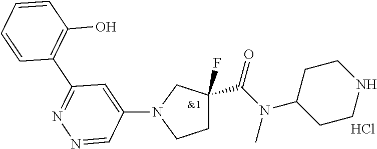 [SMILES: CN(C(=O)[C@]1(F)CCN(c2cnnc(-c3ccccc3O)c2)C1)C1CCNCC1 ; Cl]

The title compound was prepared as described for the synthesis of Example 1, Step 1, using rac-(R)-3-fluoro-1-(6-(2-(methoxymethoxy)phenyl)pyridazin-4-yl)pyrrolidine-3-carboxylic acid (Intermediate 28) and tert-butyl 4-(methylamino)piperidine-1-carboxylate to afford the title compound as a yellow oil. LCMS C₂₁H₂₆FN₅O₂ requires 399.2, found m/z=400.2 [M+H]⁺.

### Step 2: Synthesis of the Title Compound

The title compound was prepared as described for the synthesis of Example 2, Step 2 using rac-(R)-3-fluoro-1-(6-(2-hydroxyphenyl)pyridazin-4-yl)-N-methyl-N-(piperidin-4-yl)pyrrolidine-3-carboxamide hydrochloride to afford the title compound (36.1 mgs, 42% yield) as a white solid. LCMS: C₃₉H₄₈FN₇O₄ requires: 697.4, found: m/z=698.3 [M+H]⁺.

### Example 145

### (3RS)—N-{1-[2-(1-{4-[(3R)-2,6-DIOXOPIPERIDIN-3-YL]PHENYL}PIPERIDIN-4-YL)ETHYL]PIPERIDIN-4-YL}-1-[6-(2-HYDROXYPHENYL)PYRIDAZIN-4-YL]-N,3-DIMETHYLPYRROLIDINE-3-CARBOXAMIDE

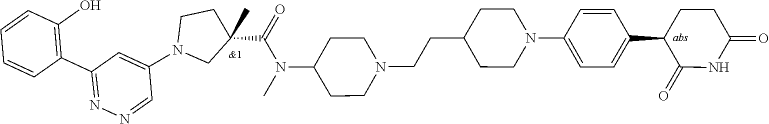 [SMILES: CN(C(=O)[C@@]1(C)CCN(c2cnnc(-c3ccccc3O)c2)C1)C1CCN(CCC2CCN(c3ccc([C@H]4CCC(=O)NC4=O)cc3)CC2)CC1]

### Step 1: rac-methyl (3R)-1-{6-[2-(methoxymethoxy)phenyl]pyridazin-4-yl}-3-methylpyrrolidine-3-carboxylate

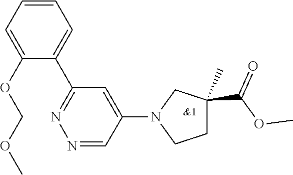

rac-methyl (3R)-3-methylpyrrolidine-3-carboxylate (158.66 μL, 169.77 mg, 1.1857 mmol), 5-chloro-3-[2-(methoxymethoxy)phenyl]pyridazine (270.20 mg, 1.0779 mmol) and N,N-diisopropylethylamine (413.05 μL, 0.31 g, 2.3713 mmol) were dissolved in DMSO (2.00 mL) and stirred in an 80° C. heating block overnight. Added more DIPEA (0.2 mL) and amine (53 μL) and heated to 100° C. Purified by RP-HPLC to yield rac-methyl (3R)-1-{6-[2-(methoxymethoxy)phenyl]pyridazin-4-yl}-3-methylpyrrolidine-3-carboxylate (0.404 g, 79.51%). LCMS: C₁₉H₂₃N₃O₄ requires: 357.2, found: m/z=358.2 [M+H]⁺.

### Step 2: rac-(3R)-1-{6-[2-(methoxymethoxy)phenyl]pyridazin-4-yl}-3-methylpyrrolidine-3-carboxylic acid

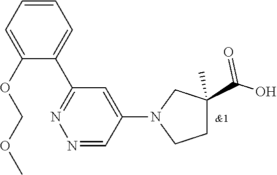

rac-methyl (3R)-1-{6-[2-(methoxymethoxy)phenyl]pyridazin-4-yl}-3-methylpyrrolidine-3-carboxylate (404.00 mg, 1.1304 mmol) was dissolved in THF (5.00 mL), then lithium hydroxide monohydrate (5.71 mL, 47.90 mg, 1.1417 mmol) was added and the reaction was stirred at rt. After 1h, added another 3 mg of LiOH and stirred overnight. THF was removed by rotovap and the remaining solution was lyophilized from 1:1 ACN/water to give the Li salt. Quant yield. LCMS: C₁₉H₂₁N₃O₄ requires: 343.2, found: m/z=344.2 [M+H]⁺.

### Step 3: rac-tert-butyl 4-[N-methyl(3R)-1-{6-[2-(methoxymethoxy)phenyl]pyridazin-4-yl}-3-methylpyrrolidine-3-amido]piperidine-1-carboxylate

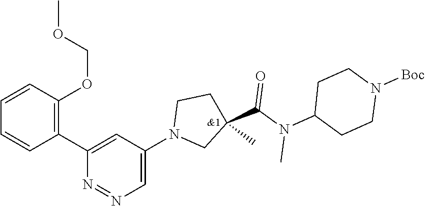 [SMILES: COCOc1ccccc1-c1cc(N2CC[C@@](C)(C(=O)N(C)C3CCN(C(=O)OC(C)(C)C)CC3)C2)cnn1]

Rac-tert-butyl 4-[N-methyl(3R)-1-{6-[2-(methoxymethoxy)phenyl]pyridazin-4-yl}-3-methylpyrrolidine-3-amido]piperidine-1-carboxylate was prepared as described for Example 107 step 1, using rac-(3R)-1-{6-[2-(methoxymethoxy)phenyl]pyridazin-4-yl}-3-methylpyrrolidine-3-carboxylic acid and tert-butyl 4-(methylamino)piperidine-1-carboxylate to yield rac-tert-butyl 4-[N-methyl(3R)-1-{6-[2-(methoxymethoxy)phenyl]pyridazin-4-yl}-3-methylpyrrolidine-3-amido]piperidine-1-carboxylate (0.0737 g, 58.62%). LCMS: C₂₉H₄₁N₅O₅ requires: 539.3, found: m/z=540.4 [M+H]⁺.

### Step 4: rac-(3R)-1-[6-(2-hydroxyphenyl)pyridazin-4-yl]-N,3-dimethyl-N-(piperidin-4-yl)pyrrolidine-3-carboxamide

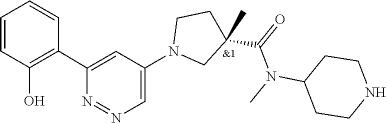 [SMILES: CN(C(=O)[C@@]1(C)CCN(c2cnnc(-c3ccccc3O)c2)C1)C1CCNCC1]

rac-(3R)-1-[6-(2-hydroxyphenyl)pyridazin-4-yl]-N,3-dimethyl-N-(piperidin-4-yl)pyrrolidine-3-carboxamide was prepared as described for Example 107 step 2. LCMS: C₂₂H₂₉N₅O₂ requires: 395.2, found: m/z=396.2 [M+H]⁺.

### Step 5: Synthesis of the Title Compound

The title compound was synthesized as described for Example 109 step 3, using rac-(3R)-1-[6-(2-hydroxyphenyl)pyridazin-4-yl]-N,3-dimethyl-N-(piperidin-4-yl)pyrrolidine-3-carboxamide hydrochloride and 2-(1-{4-[(3R)-2,6-dioxopiperidin-3-yl]phenyl}piperidin-4-yl)acetaldehyde to yield (3RS)—N-{1-[2-(1-{4-[(3R)-2,6-dioxopiperidin-3-yl]phenyl}piperidin-4-yl)ethyl]piperidin-4-yl}-1-[6-(2-hydroxyphenyl)pyridazin-4-yl]-N,3-dimethylpyrrolidine-3-carboxamide (0.0097 g, 54.54%). LCMS: C₄₀H₅₁N₇O₄ requires: 693.4, found: m/z=694.4 [M+H]⁺.

### Example 146

### (1RS&,5SR&,8RS&)—N-{1-[2-(1-{4-[(3RS)-2,6-DIOXOPIPERIDIN-3-YL]-3,5-DIFLUOROPHENYL}PIPERIDIN-4-YL)ETHYL]PIPERIDIN-4-YL}-3-[6-(2-HYDROXYPHENYL)PYRIDAZIN-4-YL]-N-(3-METHOXYPROPYL)-3-AZABICYCLO[3.2.1]OCTANE-8-CARBOXAMIDE

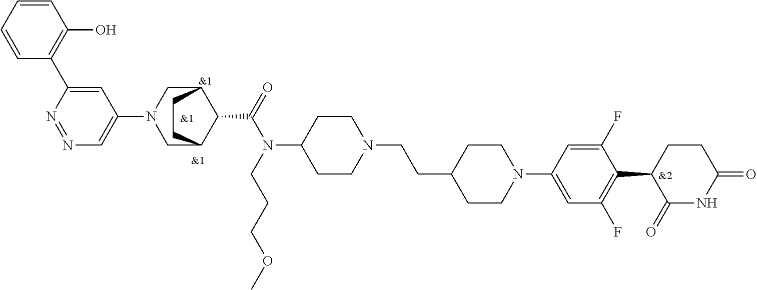 [SMILES: COCCCN(C(=O)[C@H]1[C@@H]2CC[C@H]1CN(c1cnnc(-c3ccccc3O)c1)C2)C1CCN(CCC2CCN(c3cc(F)c([C@H]4CCC(=O)NC4=O)c(F)c3)CC2)CC1]

### Step 1: tert-butyl 4-[N-(3-methoxypropyl)(1R,5S,8S)-3-{6-[2-(methoxymethoxy)phenyl]pyridazin-4-yl}-3-azabicyclo[3.2.1]octane-8-amido]piperidine-1-carboxylate

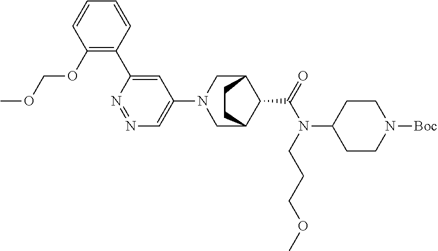 [SMILES: COCCCN(C(=O)[C@H]1[C@@H]2CC[C@H]1CN(c1cnnc(-c3ccccc3OCOC)c1)C2)C1CCN(C(=O)OC(C)(C)C)CC1]

To a solution of (1R,5S,8S)-3-{6-[2-(methoxymethoxy)phenyl]pyridazin-4-yl}-3-azabicyclo[3.2.1]octane-8-carboxylic acid (45.01 mg, 0.1218 mmol) in DMA (1.00 mL) was added tert-butyl 4-[(3-methoxypropyl)amino]piperidine-1-carboxylate (36.51 mg, 0.1340 mmol) and N,N-diisopropylethylamine (63.67 μL, 0.05 g, 0.3655 mmol). [(dimethylamino)({[1,2,3]triazolo[4,5-b]pyridin-3-yloxy})methylidene]dimethylazanium; hexafluoro-lambda5-phosphanuide (55.59 mg, 0.1462 mmol) was added. Stirred at rt for 2 h, then added more HATU (55 mg) and stirred overnight. Purified directly by RP-HPLC to yield tert-butyl 4-[N-(3-methoxypropyl)(1R,5S,8S)-3-{6-[2-(methoxymethoxy)phenyl]pyridazin-4-yl}-3-azabicyclo[3.2.1]octane-8-amido]piperidine-1-carboxylate (0.068 g, 75.64%). LCMS: C₃₄H₄₉N₅O₆ requires: 623.4, found: m/z=624.4 [M+H]⁺.

### Step 2: (1R,5S,8S)-3-[6-(2-hydroxyphenyl)pyridazin-4-yl]-N-(3-methoxypropyl)-N-(piperidin-4-yl)-3-azabicyclo[3.2.1]octane-8-carboxamide

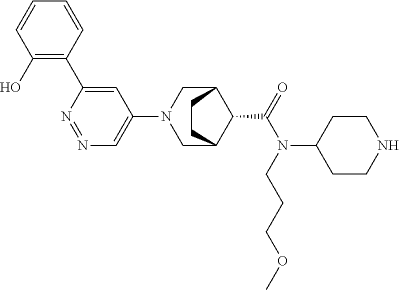 [SMILES: COCCCN(C(=O)[C@H]1[C@@H]2CC[C@H]1CN(c1cnnc(-c3ccccc3O)c1)C2)C1CCNCC1]

Tert-butyl 4-[N-(3-methoxypropyl)(1R,5S,8S)-3-{6-[2-(methoxymethoxy)phenyl]pyridazin-4-yl}-3-azabicyclo[3.2.1]octane-8-amido]piperidine-1-carboxylate (68.00 mg, 0.1090 mmol) was dissolved in TFA (4.00 mL). After 90 min the reaction was concentrated down to an oil, then lyophilized from 1:1 water/ACN. Tris TFA salt based on a quantitative yield. LCMS: C₂₇H₃₇N₅O₃ requires: 479.3, found: m/z=480.2 [M+H]⁺.

### Step: 3 Synthesis of the Title Compound

Combined (1R,5S,8S)-3-[6-(2-hydroxyphenyl)pyridazin-4-yl]-N-(3-methoxypropyl)-N-(piperidin-4-yl)-3-azabicyclo[3.2.1]octane-8-carboxamide; tris(trifluoroacetic acid) (15.10 mg, 0.0184 mmol) and rac-2-(1-{4-[(3R)-2,6-dioxopiperidin-3-yl]-3,5-difluorophenyl}piperidin-4-yl)acetaldehyde (6.76 mg, 0.0193 mmol) in DMA (0.40 mL) with N,N-diisopropylethylamine (9.63 μL, 0.01 g, 0.0551 mmol) and then added sodium triacetoxyborohydride (7.01 mg, 0.0331 mmol) and stirred for 1h. Purified the reaction directly by RP-HPLC to yield (1RS&,5SR&,8RS&)—N-{1-[2-(1-{4-[(3RS)-2,6-dioxopiperidin-3-yl]-3,5-difluorophenyl}piperidin-4-yl)ethyl]piperidin-4-yl}-3-[6-(2-hydroxyphenyl)pyridazin-4-yl]-N-(3-methoxypropyl)-3-azabicyclo[3.2.1]octane-8-carboxamide (0.0082 g, 50.38%). LCMS: C₄₅H₅₇F₂N₇O₅ requires: 813.4, found: m/z=814.4 [M+H]⁺.

### Example 147

### N-{1-[2-(1-{4-[(3R)-2,6-DIOXOPIPERIDIN-3-YL]-3-FLUOROPHENYL}PIPERIDIN-4-YL)ETHYL]PIPERIDIN-4-YL}-1-[6-(2-HYDROXYPHENYL)PYRIDAZIN-4-YL]-N-(2-METHOXYETHYL)-4-METHYLPIPERIDINE-4-CARBOXAMIDE

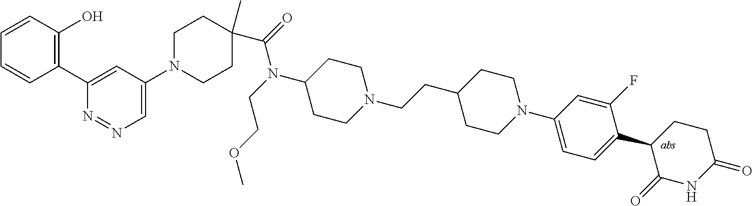 [SMILES: COCCN(C(=O)C1(C)CCN(c2cnnc(-c3ccccc3O)c2)CC1)C1CCN(CCC2CCN(c3ccc([C@H]4CCC(=O)NC4=O)c(F)c3)CC2)CC1]

4.8 mg of Example 139 was subjected to SFC chiral separation. Peak 2 is Example 147.

| Name | Yield(mg) | Purity(%) | Column Used |
|---|---|---|---|
| Ex 139 pk 1 | 2.1 | 96.4 | Whelk-O1(R,R) |
| Ex. 139 pk 2 | 2.1 | 97.6 | Whelk-O1(R,R) |

LCMS: C₄₃H₅₆FN₇O₅ requires: 769.4, found: m/z=770.3 [M+H]⁺.

### Example 148

### (R)—N-CYCLOPROPYL-N-(1-(2-(1-(4-(2,6-DIOXOPIPERIDIN-3-YL)PHENYL)PIPERIDIN-4-YL)ETHYL)PIPERIDIN-4-YL)-1-(6-(5-FLUORO-2-HYDROXYPHENYL)PYRIDAZIN-4-YL)-4-(METHOXYMETHYL)PIPERIDINE-4-CARBOXAMIDE

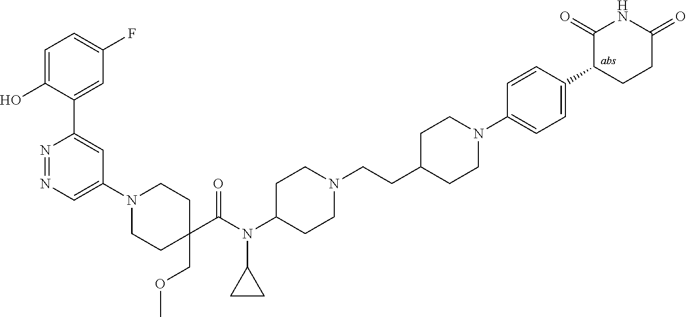 [SMILES: COCC1(C(=O)N(C2CC2)C2CCN(CCC3CCN(c4ccc([C@H]5CCC(=O)NC5=O)cc4)CC3)CC2)CCN(c2cnnc(-c3cc(F)ccc3O)c2)CC1]

### Step 1: Synthesis of tert-butyl 4-(1-(6-chloropyridazin-4-yl)-N-cyclopropyl-4-(methoxymethyl)piperidine-4-carboxamido)piperidine-1-carboxylate

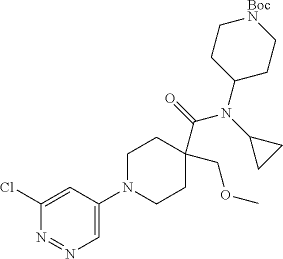 [SMILES: COCC1(C(=O)N(C2CC2)C2CCN(C(=O)OC(C)(C)C)CC2)CCN(c2cnnc(Cl)c2)CC1]

tert-butyl 4-[N-cyclopropyl-4-(methoxymethyl)piperidine-4-amido]piperidine-1-carboxylate (220.00 mg, 0.5562 mmol) and 3,5-dichloropyridazine (82.86 mg, 0.5562 mmol) were dissolved in 3 mL DMSO under argon atmosphere. N,N-diisopropylethylamine (0.48 mL, 359.43 mg, 2.7810 mmol) was then added and the resulting solution was stirred at 100° C. overnight. The reaction was monitored via LCMS. Once complete, the reaction was concentrated to remove excess DIPEA and then quenched with H₂O and extracted with EtOAc. The organic phases were combined, dried over sodium sulfate and concentrated in vacuo. The resulting residue was purified on silica gel (EtOAc/hexanes gradient) to afford the desired product (188 mg, 66.5%) as a pink solid.

### Step 2: Synthesis of N-cyclopropyl-1-(6-(5-fluoro-2-hydroxyphenyl)pyridazin-4-yl)-4-(methoxymethyl)-N-(piperidin-4-yl)piperidine-4-carboxamide

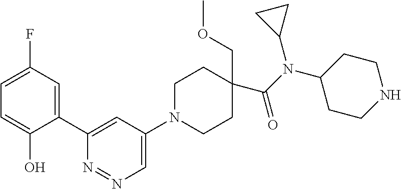 [SMILES: COCC1(C(=O)N(C2CCNCC2)C2CC2)CCN(c2cnnc(-c3cc(F)ccc3O)c2)CC1]

A flask was charged with tert-butyl 4-[N-cyclopropyl-1-(6-chloropyridazin-4-yl)-4-(methoxymethyl)piperidine-4-amido]piperidine-1-carboxylate (188.00 mg, 0.3700 mmol), potassium carbonate (153.42 mg, 1.1101 mmol), and 4-fluoro-2-hydroxyphenylboronic acid (57.70 mg, 0.3700 mmol). 1,4-Dioxane (3.75 mL) and Water (1.25 mL) were sequentially added to the flask. The resulting solution was degassed with an Argon balloon for 5 minutes and then tetrakis(triphenylphosphine)palladium(O) (42.76 mg, 0.0370 mmol) was added in one portion. The flask was sealed, and the resulting mixture was stirred at 100° C. for 18 h. Upon completion, the flask was diluted in DCM and sat. NH4Cl was added. The mixture was extracted 3× with DCM and the organic phases were dried over sodium sulfate, concentrated in vacuo and purified on silica gel to afford the BOC-protected product as a yellow solid. The obtained material was then dissolved in MeOH (1.0 mL) and 4M HCl in dioxane (1.0 mL) was added in one portion. The resulting solution was stirred at RT for 1 hour and monitored via LCMS. Once complete, the reaction was concentrated in vacuo to afford the desired product (75 mg, 45.1%) as a tan solid. LCMS: C₂₆H₃₄FN₅O₃ requires: 483.2, found: m/z=484.2 [M+H]⁺.

### Step 3: Synthesis of the Title Compound

N-cyclopropyl-1-[6-(5-fluoro-2-hydroxyphenyl)pyridazin-4-yl]-4-(methoxymethyl)-N-(piperidin-4-yl)piperidine-4-carboxamide hydrochloride (20.00 mg, 0.0385 mmol) was dissolved in MeCN (0.45 mL) and 2-(1-{4-[(3R)-2,6-dioxopiperidin-3-yl]phenyl}piperidin-4-yl)acetaldehyde (13.30 mg, 0.0423 mmol) was added in one portion. The flask was agitated and then MeOH (0.13 mL) was slowly added until all the solids have dissolved. Sodium triacetoxyborohydride (24.45 mg, 0.1154 mmol) was then added in one portion and the reaction was stirred at RT for 30 mins. After 30 mins, the reaction was checked by LCMS and the reaction mixture was dissolved in DCM and water was added. The reaction was extracted 3× with DCM and concentrated in vacuo. The residue was then purified by RP—FC to yield the title compound (29.2 mg, 57.57%) as a formate salt. LCMS: C₄₄H₅₆FN₇O₅ requires: 781.4, found: m/z=782.3 [M+H]⁺.

### Example 149

### ((R)—N-(1-(2-(1-(4-(2,6-DIOXOPIPERIDIN-3-YL)PHENYL)PIPERIDIN-4-YL)ETHYL)PIPERIDIN-4-YL)-N-ETHYL-1-(6-(5-FLUORO-2-HYDROXYPHENYL)PYRIDAZIN-4-YL)-4-(METHOXYMETHYL)PIPERIDINE-4-CARBOXAMIDE

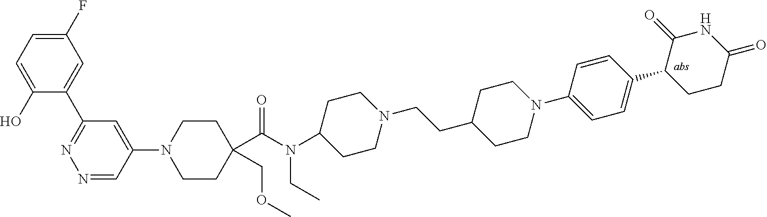 [SMILES: CCN(C(=O)C1(COC)CCN(c2cnnc(-c3cc(F)ccc3O)c2)CC1)C1CCN(CCC2CCN(c3ccc([C@H]4CCC(=O)NC4=O)cc3)CC2)CC1]

### Step 1: Synthesis of tert-butyl 4-(1-(6-chloropyridazin-4-yl)-N-ethyl-4-(methoxymethyl)piperidine-4-carboxamido)piperidine-1-carboxylate

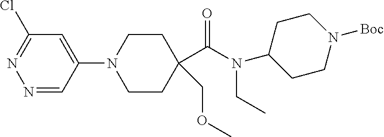 [SMILES: CCN(C(=O)C1(COC)CCN(c2cnnc(Cl)c2)CC1)C1CCN(C(=O)OC(C)(C)C)CC1]

tert-butyl 4-[N-ethyl-4-(methoxymethyl)piperidine-4-amido]piperidine-1-carboxylate (260.00 mg, 0.6779 mmol) and 3,5-dichloropyridazine (100.99 mg, 0.6779 mmol) were dissolved in 0.5M DMSO under argon atmosphere. N,N-diisopropylethylamine (0.59 mL, 438.09 mg, 3.3895 mmol) was then added and the resulting solution was stirred at 100° C. overnight. The reaction was monitored via LCMS. Once complete, the reaction was concentrated to remove excess DIPEA and then quenched with H₂O and extracted with EtOAc. The organic phases were combined, dried over sodium sulfate and concentrated in vacuo. The resulting residue was purified on silica gel (EtOAc/hexanes gradient) to afford the desired product (217 mg, 64.5%) as an orange solid.

### Step 2: Synthesis of N-ethyl-1-(6-(5-fluoro-2-hydroxyphenyl)pyridazin-4-yl)-4-(methoxymethyl)-N-(piperidin-4-yl)piperidine-4-carboxamide

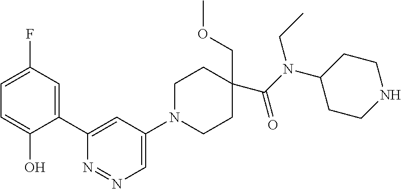 [SMILES: CCN(C(=O)C1(COC)CCN(c2cnnc(-c3cc(F)ccc3O)c2)CC1)C1CCNCC1]

A flask was charged with tert-butyl 4-[N-ethyl-1-(6-chloropyridazin-4-yl)-4-(methoxymethyl)piperidine-4-amido]piperidine-1-carboxylate (110.00 mg, 0.2218 mmol), potassium carbonate (91.94 mg, 0.6653 mmol), and 4-fluoro-2-hydroxyphenylboronic acid (34.58 mg, 0.2218 mmol). 1,4-Dioxane (2.50 mL) and Water (1.00 mL) were sequentially added to the flask. The resulting solution was degassed with an Argon balloon for 5 mins and then tetrakis(triphenylphosphine)palladium(O) (25.63 mg, 0.0222 mmol) was added in one portion. The flask was sealed, and the resulting mixture was stirred at 100° C. for 18 h. Upon completion, the flask was diluted in DCM and sat. NH4Cl was added. The mixture was extracted 3× with DCM and the organic phases were dried over sodium sulfate, concentrated in vacuo and purified on silica gel to afford the BOC-protected product as a yellow solid. The obtained material was then dissolved in MeOH (1.0 mL) and 4M HCl in dioxane (1.0 mL) was added in one portion. The resulting solution was stirred at RT for 1 hour and monitored via LCMS. Once complete, the reaction was concentrated in vacuo to afford the desired product (66 mg) as a tan solid. LCMS: C₂₅H₃₄FN₅O₃ requires: 471.2, found: m/z=472.3 [M+H]⁺.

### Step 3: Synthesis of the Title Compound

N-ethyl-1-[6-(5-fluoro-2-hydroxyphenyl)pyridazin-4-yl]-4-(methoxymethyl)-N-(piperidin-4-yl)piperidine-4-carboxamide hydrochloride (20.00 mg, 0.0394 mmol) was dissolved in MeCN (0.45 mL) and 2-(1-{4-[(3R)-2,6-dioxopiperidin-3-yl]phenyl}piperidin-4-yl)acetaldehyde (13.61 mg, 0.0433 mmol) was added in one portion. The flask was agitated and then MeOH (0.13 mL) was slowly added until all the solids have dissolved. Sodium triacetoxyborohydride (25.03 mg, 0.1181 mmol) was then added in one portion and the reaction was stirred at RT for 30 mins. After 30 mins, the reaction was checked by LCMS and the reaction mixture was dissolved in DCM and water was added. The reaction was extracted 3× with DCM and concentrated in vacuo. The residue was then purified by RP—FC to yield the title compound (9.5 mg, 29.5%) as a formate salt. LCMS: C₄₃H₅₆FN₇O₅ requires: 769.4, found: m/z=770.3 [M+H]⁺.

### Example 150

### (1RS,5SR,8RS)—N-{1-[2-(1-{4-[(3R)-2,6-DIOXOPIPERIDIN-3-YL]-3-FLUOROPHENYL}PIPERIDIN-4-YL)ETHYL]PIPERIDIN-4-YL}-3-[6-(2-HYDROXYPHENYL)PYRIDAZIN-4-YL]-N-(3-METHOXYPROPYL)-3-AZABICYCLO[3.2.1]OCTANE-8-CARBOXAMIDE

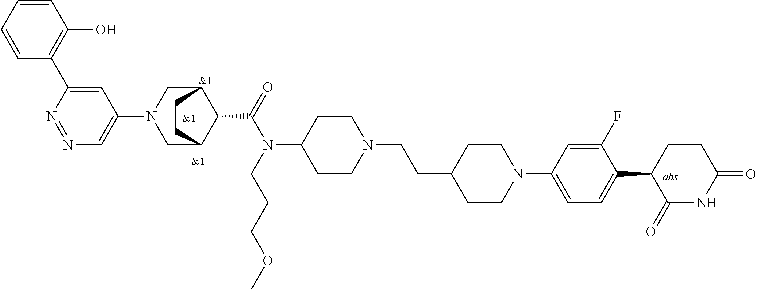 [SMILES: COCCCN(C(=O)[C@H]1[C@@H]2CC[C@H]1CN(c1cnnc(-c3ccccc3O)c1)C2)C1CCN(CCC2CCN(c3ccc([C@H]4CCC(=O)NC4=O)c(F)c3)CC2)CC1]

Combined (1R,5S,8S)-3-[6-(2-hydroxyphenyl)pyridazin-4-yl]-N-(3-methoxypropyl)-N-(piperidin-4-yl)-3-azabicyclo[3.2.1]octane-8-carboxamide; tris(trifluoroacetic acid) (15.10 mg, 0.0184 mmol) and 2-(1-{4-[(3R)-2,6-dioxopiperidin-3-yl]-3-fluorophenyl}piperidin-4-yl)acetaldehyde (6.41 mg, 0.0193 mmol) in DMA (0.40 mL) with N,N-diisopropylethylamine (9.63 μL, 0.01 g, 0.0551 mmol) and then added sodium triacetoxyborohydride (7.01 mg, 0.0331 mmol) and stirred for 1h. Purified the reaction directly by RP-HPLC (1RS,5SR,8RS)—N-{1-[2-(1-{4-[(3R)-2,6-dioxopiperidin-3-yl]-3-fluorophenyl}piperidin-4-yl)ethyl]piperidin-4-yl}-3-[6-(2-hydroxyphenyl)pyridazin-4-yl]-N-(3-methoxypropyl)-3-azabicyclo[3.2.1]octane-8-carboxamide (0.0086 g, 53.41%). LCMS: C₄₅H₅₈FN₇O₅ requires: 795.4, found: m/z=796.3 [M+H]⁺.

### Example 151

### (R)—N-(1-(2-(1-(5-(2,6-DIOXOPIPERIDIN-3-YL)PYRIDIN-2-YL)PIPERIDIN-4-YL)ETHYL)PIPERIDIN-4-YL)-N-ETHYL-1-(6-(5-FLUORO-2-HYDROXYPHENYL)PYRIDAZIN-4-YL)-4-(METHOXYMETHYL)PIPERIDINE-4-CARBOXAMIDE

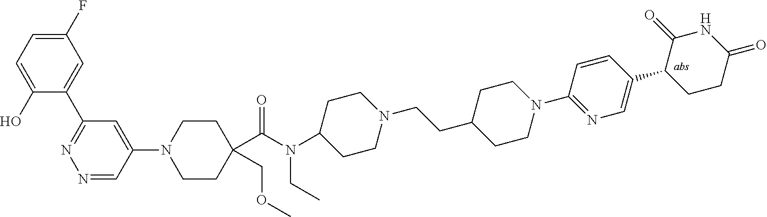 [SMILES: CCN(C(=O)C1(COC)CCN(c2cnnc(-c3cc(F)ccc3O)c2)CC1)C1CCN(CCC2CCN(c3ccc([C@H]4CCC(=O)NC4=O)cn3)CC2)CC1]

### Step 1: Synthesis of the Title Compound

N-ethyl-1-[6-(5-fluoro-2-hydroxyphenyl)pyridazin-4-yl]-4-(methoxymethyl)-N-(piperidin-4-yl)piperidine-4-carboxamide hydrochloride (10.00 mg, 0.0192 mmol) was dissolved in DMA (1.00 mL) and rac-2-(1-{5-[(3R)-2,6-dioxopiperidin-3-yl]pyridin-2-yl}piperidin-4-yl)acetaldehyde (6.39 mg, 0.0192 mmol) was added in one portion. N,N-diisopropylethylamine (12.43 mg, 0.0961 mmol) was then added followed by sodium triacetoxyborohydride (12.23 mg, 0.0577 mmol). The resulting reaction was stirred at RT for 1 hour and monitored via LCMS. Once complete, the reaction was concentrated in vacuo and purified via RP—FC to afford the title compound (2.3 mg, 14.3%) as a formate salt. LCMS: C₄₂H₅₅FN₈O₅ requires: 770.4, found: m/z=771.4 [M+H]⁺.

### Example 152

### (R)—N-(1-(2-(1-(4-((R)-2,6-DIOXOPIPERIDIN-3-YL)PHENYL)PIPERIDIN-4-YL)ETHYL)PIPERIDIN-4-YL)-3-FLUORO-1-(6-(2-HYDROXYPHENYL)PYRIDAZIN-4-YL)-N-METHYLPYRROLIDINE-3-CARBOXAMIDE

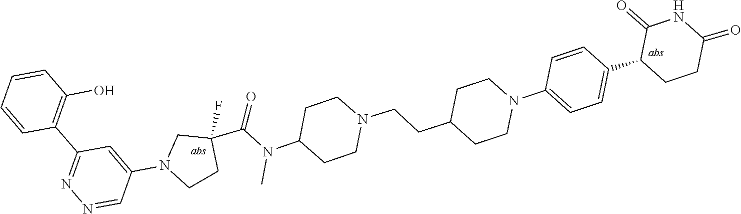 [SMILES: CN(C(=O)[C@@]1(F)CCN(c2cnnc(-c3ccccc3O)c2)C1)C1CCN(CCC2CCN(c3ccc([C@H]4CCC(=O)NC4=O)cc3)CC2)CC1]

### Step 1: Synthesis of the Title Compound

(RS)—N-(1-(2-(1-(4-((R)-2,6-dioxopiperidin-3-yl)phenyl)piperidin-4-yl)ethyl)piperidin-4-yl)-3-fluoro-1-(6-(2-hydroxyphenyl)pyridazin-4-yl)-N-methylpyrrolidine-3-carboxamide (Example 144, 36.1 mgs, 0.052 mmol) was subjected to SFC chromatography (first-eluting isomer, RT=3.32 min, ChiralPak IH, EtOH+60ACN+0.1% NH₄OH) to afford the title compound (15.1 mgs, 42% yield) as a white solid. LCMS: C₃₉H₄₈FN₇O₄ requires: 697.4, found: m/z=698.3 [M+H]⁺.

### Example 153

### (1R,5S,8R)—N-{1-[2-(1-{4-[(3R)-2,6-DIOXOPIPERIDIN-3-YL]PHENYL}PIPERIDIN-4-YL)ETHYL]PIPERIDIN-4-YL}-N-ETHYL-3-[6-(2-HYDROXYPHENYL)PYRIDAZIN-4-YL]-3-AZABICYCLO[3.2.1]OCTANE-8-CARBOXAMIDE

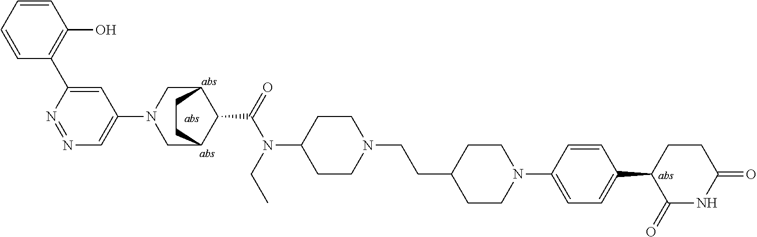 [SMILES: CCN(C(=O)[C@H]1[C@@H]2CC[C@H]1CN(c1cnnc(-c3ccccc3O)c1)C2)C1CCN(CCC2CCN(c3ccc([C@H]4CCC(=O)NC4=O)cc3)CC2)CC1]

### Step 1: methyl (1R,5S,8S)-3-{6-[2-(methoxymethoxy)phenyl]pyridazin-4-yl}-3-azabicyclo[3.2.1]octane-8-carboxylate

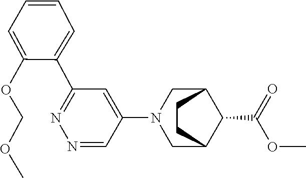 [SMILES: COCOc1ccccc1-c1cc(N2C[C@H]3CC[C@@H](C2)[C@H]3C(=O)OC)cnn1]

Methyl (1R,5S,8R)-3-azabicyclo[3.2.1]octane-8-carboxylate hydrochloride (492.29 mg, 2.3935 mmol), 5-chloro-3-[2-(methoxymethoxy)phenyl]pyridazine (400.00 mg, 1.5957 mmol) and N,N-diisopropylethylamine (833.83 μL, 0.62 g, 4.7870 mmol) were dissolved in DMSO (5.00 mL) and stirred in an 80° C. heating block overnight. Added more DIPEA (0.2 mL) and amine (53 μL) and heated to 100° C. and then purified by RP-HPLC to yield methyl (1R,5S,8S)-3-{6-[2-(methoxymethoxy)phenyl]pyridazin-4-yl}-3-azabicyclo[3.2.1]octane-8-carboxylate (0.482 g, 60.72%). LCMS: C₂₁H₂₅N₃O₄ requires: 383.2, found: m/z=384.2 [M+H]⁺.

### Step 2: (1R,5S,8S)-3-{6-[2-(methoxymethoxy)phenyl]pyridazin-4-yl}-3-azabicyclo[3.2.1]octane-8-carboxylic acid

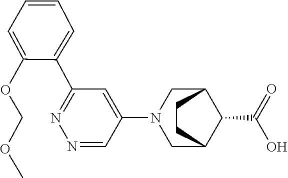 [SMILES: COCOc1ccccc1-c1cc(N2C[C@H]3CC[C@@H](C2)[C@H]3C(=O)O)cnn1]

methyl (1R,5S,8S)-3-{6-[2-(methoxymethoxy)phenyl]pyridazin-4-yl}-3-azabicyclo[3.2.1]octane-8-carboxylate (482.00 mg, 1.2570 mmol) was dissolved in THF (6.00 mL) and 0.2 M lithium hydroxide monohydrate (6.29 mL, 52.74 mg, 1.2570 mmol). After an hour more 0.2 M lithium hydroxide monohydrate (6.29 mL, 52.74 mg, 1.2570 mmol) was added. After another hour, the THF was removed and the solution was frozen and lyo'd. Quant yield. LCMS: C₂₀H₂₃N₃O₄ requires: 369.2, found: m/z=370.2 [M+H]⁺.

### Step 3: tert-butyl 4-[N-ethyl(1R,5S,8S)-3-{6-[2-(methoxymethoxy)phenyl]pyridazin-4-yl}-3-azabicyclo[3.2.1]octane-8-amido]piperidine-1-carboxylate

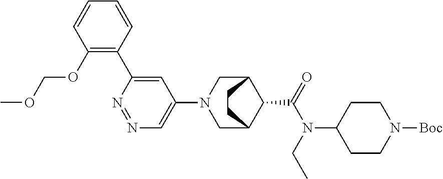 [SMILES: CCN(C(=O)[C@H]1[C@@H]2CC[C@H]1CN(c1cnnc(-c3ccccc3OCOC)c1)C2)C1CCN(C(=O)OC(C)(C)C)CC1]

To a solution of (1R,5S,8S)-3-{6-[2-(methoxymethoxy)phenyl]pyridazin-4-yl}-3-azabicyclo[3.2.1]octane-8-carboxylic acid (150.50 mg, 0.4074 mmol) in DMA (1.00 mL) was added tert-butyl 4-(ethylamino)piperidine-1-carboxylate (139.53 mg, 0.6111 mmol) and N,N-diisopropylethylamine (212.89 μL, 0.16 g, 1.2222 mmol). [(dimethylamino)({[1,2,3]triazolo[4,5-b]pyridin-3-yloxy})methylidene]dimethylazanium; hexafluoro-lambda5-phosphanuide (185.89 mg, 0.4889 mmol) was added and the reaction was stirred at rt for 2h. Purified directly by RP-HPLC to yield tert-butyl 4-[N-ethyl(1R,5S,8S)-3-{6-[2-(methoxymethoxy)phenyl]pyridazin-4-yl}-3-azabicyclo[3.2.1]octane-8-amido]piperidine-1-carboxylate (0.127 g, 44.93%). LCMS: C₃₂H₄₅N₅O₅ requires: 579.3, found: m/z=580.4 [M+H]⁺.

### Step 4: (1R,5S,8S)—N-ethyl-3-[6-(2-hydroxyphenyl)pyridazin-4-yl]-N-(piperidin-4-yl)-3-azabicyclo[3.2.1]octane-8-carboxamide

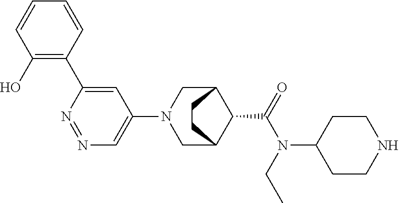 [SMILES: CCN(C(=O)[C@H]1[C@@H]2CC[C@H]1CN(c1cnnc(-c3ccccc3O)c1)C2)C1CCNCC1]

(1R,5S,8S)—N-ethyl-3-[6-(2-hydroxyphenyl)pyridazin-4-yl]-N-(piperidin-4-yl)-3-azabicyclo[3.2.1]octane-8-carboxamide was prepared as described for Example 107 step 2. LCMS: C₂₅H₃₃N₅O₂ requires: 435.3, found: m/z=436.2 [M+H]⁺.

### Step 5: Synthesis of the Title Compound

Combined (1R,5S,8S)—N-ethyl-3-[6-(2-hydroxyphenyl)pyridazin-4-yl]-N-(piperidin-4-yl)-3-azabicyclo[3.2.1]octane-8-carboxamide hydrochloride (12.00 mg, 0.0254 mmol) and 2-(1-{4-[(3R)-2,6-dioxopiperidin-3-yl]phenyl}piperidin-4-yl)acetaldehyde (8.79 mg, 0.0280 mmol) in DMA (0.30 mL) and then added N,N-diisopropylethylamine (13.32 μL, 0.01 g, 0.0763 mmol) and sodium triacetoxyborohydride (8.08 mg, 0.0381 mmol) and stirred for 2 h, then purified directly by RP-HPLC to yield (1R,5S,8R)—N-{1-[2-(1-{4-[(3R)-2,6-dioxopiperidin-3-yl]phenyl}piperidin-4-yl)ethyl]piperidin-4-yl}-N-ethyl-3-[6-(2-hydroxyphenyl)pyridazin-4-yl]-3-azabicyclo[3.2.1]octane-8-carboxamide (0.014 g, 70.18%). LCMS: C₄₃H₅₅N₇O₄ requires: 733.4, found: m/z=734.2 [M+H]⁺.

### Example 154

### (1R,5S,8R)—N-{1-[2-(1-{4-[(3RS)-2,6-DIOXOPIPERIDIN-3-YL]-3-FLUOROPHENYL}PIPERIDIN-4-YL)ETHYL]PIPERIDIN-4-YL}-N-ETHYL-3-[6-(2-HYDROXYPHENYL)PYRIDAZIN-4-YL]-3-AZABICYCLO[3.2.1]OCTANE-8-CARBOXAMIDE

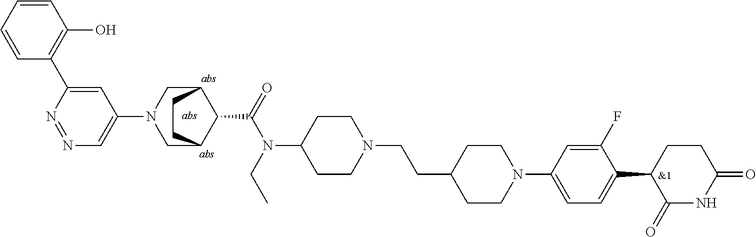 [SMILES: CCN(C(=O)[C@H]1[C@@H]2CC[C@H]1CN(c1cnnc(-c3ccccc3O)c1)C2)C1CCN(CCC2CCN(c3ccc([C@H]4CCC(=O)NC4=O)c(F)c3)CC2)CC1]

The title compound was synthesized as described for Example 153 step 5, using rac-2-(1-{4-[(3R)-2,6-dioxopiperidin-3-yl]-3-fluorophenyl}piperidin-4-yl)acetaldehyde to yield (1R,5S,8R)—N-{1-[2-(1-{4-[(3RS)-2,6-dioxopiperidin-3-yl]-3-fluorophenyl}piperidin-4-yl)ethyl]piperidin-4-yl}-N-ethyl-3-[6-(2-hydroxyphenyl)pyridazin-4-yl]-3-azabicyclo[3.2.1]octane-8-carboxamide (0.0146 g, 70.96%). LCMS: C₄₃H₅₄FN₇O₄ requires: 751.4, found: m/z=752.2 [M+H]⁺.

### Example 155

### (S)—N-(1-(2-(1-(4-((R)-2,6-DIOXOPIPERIDIN-3-YL)-3-FLUOROPHENYL)PIPERIDIN-4-YL)ETHYL)PIPERIDIN-4-YL)-3-FLUORO-1-(6-(2-HYDROXYPHENYL)PYRIDAZIN-4-YL)-N-METHYLPYRROLIDINE-3-CARBOXAMIDE

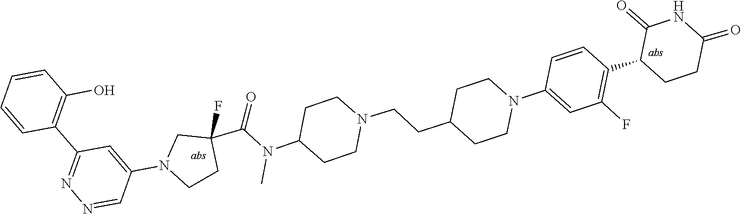 [SMILES: CN(C(=O)[C@]1(F)CCN(c2cnnc(-c3ccccc3O)c2)C1)C1CCN(CCC2CCN(c3ccc([C@H]4CCC(=O)NC4=O)c(F)c3)CC2)CC1]

### Step 1: Synthesis of the Title Compound

The title compound was prepared as described for the synthesis of Example 1, Step 2, using rac-(R)-3-fluoro-1-(6-(2-hydroxyphenyl)pyridazin-4-yl)-N-methyl-N-(piperidin-4-yl)pyrrolidine-3-carboxamide hydrochloride (Example 144, Step 1). After RP-HPLC, the compound was subjected to two stages of SFC purification (Whelk-O1 (R,R), EtOH+60ACN+0.1% NH₄OH followed by Chiralpak IBN, EtOH+60ACN+0.1% NH₄OH) to afford the title compound (Peak 1 after Stage 2 SFC, RT=3.92 min, 11.6 mgs, 10% yield) as a white solid. Assignment of absolute stereochemistry is arbitrary. LCMS C₃₉H₄₇F₂N₇O₄ requires 715.4, found m/z=716.4 [M+H]⁺.

### Example 156

### (1R,5S,8R)—N-{1-[2-(1-{4-[(3R)-2,6-DIOXOPIPERIDIN-3-YL]PHENYL}PIPERIDIN-4-YL)ETHYL]PIPERIDIN-4-YL}-3-[6-(2-HYDROXYPHENYL)PYRIDAZIN-4-YL]-N-METHYL-3-AZABICYCLO[3.2.1]OCTANE-8-CARBOXAMIDE

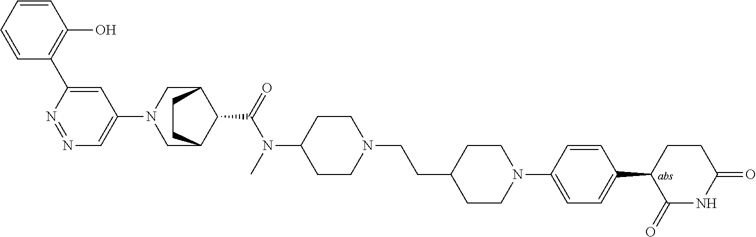 [SMILES: CN(C(=O)[C@H]1[C@@H]2CC[C@H]1CN(c1cnnc(-c3ccccc3O)c1)C2)C1CCN(CCC2CCN(c3ccc([C@H]4CCC(=O)NC4=O)cc3)CC2)CC1]

### Step 1: tert-butyl 4-[N-methyl(1R,5S,8S)-3-{6-[2-(methoxymethoxy)phenyl]pyridazin-4-yl}-3-azabicyclo[3.2.1]octane-8-amido]piperidine-1-carboxylate

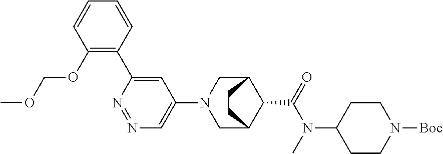 [SMILES: COCOc1ccccc1-c1cc(N2C[C@H]3CC[C@@H](C2)[C@H]3C(=O)N(C)C2CCN(C(=O)OC(C)(C)C)CC2)cnn1]

Tert-butyl 4-[N-methyl(1R,5S,8S)-3-{6-[2-(methoxymethoxy)phenyl]pyridazin-4-yl}-3-azabicyclo[3.2.1]octane-8-amido]piperidine-1-carboxylate was synthesized as described for Example 153 step 3, using (1R,5S,8S)-3-{6-[2-(methoxymethoxy)phenyl]pyridazin-4-yl}-3-azabicyclo[3.2.1]octane-8-carboxylic acid and tert-butyl 4-(methylamino)piperidine-1-carboxylate to yield tert-butyl 4-[N-methyl(1R,5S,8S)-3-{6-[2-(methoxymethoxy)phenyl]pyridazin-4-yl}-3-azabicyclo[3.2.1]octane-8-amido]piperidine-1-carboxylate (0.0509 g, 55.40%). LCMS: C₃₁H₄₃N₅O₅ requires: 565.3, found: m/z=566.4 [M+H]⁺.

### Step 2: (1R,5S,8S)-3-[6-(2-hydroxyphenyl)pyridazin-4-yl]-N-methyl-N-(piperidin-4-yl)-3-azabicyclo[3.2.1]octane-8-carboxamide

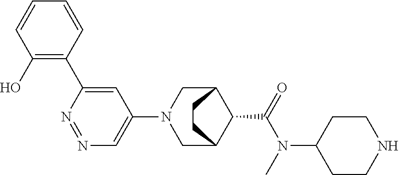 [SMILES: CN(C(=O)[C@H]1[C@@H]2CC[C@H]1CN(c1cnnc(-c3ccccc3O)c1)C2)C1CCNCC1]

(1R,5S,8S)-3-[6-(2-hydroxyphenyl)pyridazin-4-yl]-N-methyl-N-(piperidin-4-yl)-3-azabicyclo[3.2.1]octane-8-carboxamide was prepared as described for Example 107 step 2. LCMS: C₂₄H₃₁N₅O₂ requires: 421.2, found: m/z=422.2 [M+H]⁺.

### Step 3: Synthesis of the Title Compound

The title compound was synthesized as described for Example 153 step 5, using (1R,5S,8S)-3-[6-(2-hydroxyphenyl)pyridazin-4-yl]-N-methyl-N-(piperidin-4-yl)-3-azabicyclo[3.2.1]octane-8-carboxamide hydrochloride and 2-(1-{4-[(3R)-2,6-dioxopiperidin-3-yl]phenyl}piperidin-4-yl)acetaldehyde to yield (1R,5S,8R)—N-{1-[2-(1-{4-[(3R)-2,6-dioxopiperidin-3-yl]phenyl}piperidin-4-yl)ethyl]piperidin-4-yl}-3-[6-(2-hydroxyphenyl)pyridazin-4-yl]-N-methyl-3-azabicyclo[3.2.1]octane-8-carboxamide (0.0064 g, 38.27%). LCMS: C₄₂H₅₃N₇O₄ requires: 719.4, found: m/z=720.2 [M+H]⁺.

### Example 157

### (R)—N-(1-(2-(1-(4-((R)-2,6-DIOXOPIPERIDIN-3-YL)-3-FLUOROPHENYL)PIPERIDIN-4-YL)ETHYL)PIPERIDIN-4-YL)-3-FLUORO-1-(6-(2-HYDROXYPHENYL)PYRIDAZIN-4-YL)-N-METHYLPYRROLIDINE-3-CARBOXAMIDE

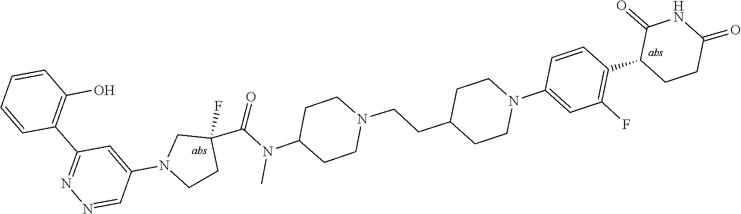 [SMILES: CN(C(=O)[C@@]1(F)CCN(c2cnnc(-c3ccccc3O)c2)C1)C1CCN(CCC2CCN(c3ccc([C@H]4CCC(=O)NC4=O)c(F)c3)CC2)CC1]

The title compound was prepared as described for the synthesis of Example 1, Step 2, using rac-(R)-3-fluoro-1-(6-(2-hydroxyphenyl)pyridazin-4-yl)-N-methyl-N-(piperidin-4-yl)pyrrolidine-3-carboxamide hydrochloride (Example 144, Step 1). After RP-HPLC, the compound was subjected to two stages of SFC purification (Whelk-O1 (R,R), EtOH+60ACN+0.1% NH₄OH followed by Chiralpak IBN, EtOH+60ACN+0.1% NH₄OH) to afford the title compound (Peak 1 after Stage 2 SFC, RT=3.99 min, 10.8 mgs, 9% yield) as a white solid. Assignment of absolute stereochemistry is arbitrary. LCMS C₃₉H₄₇F₂N₇O₄ requires 715.4, found m/z=716.4 [M+H]⁺.

### Example 158

### (1R,5S,8R)—N-{1-[2-(1-{4-[(3RS)-2,6-DIOXOPIPERIDIN-3-YL]-3-FLUOROPHENYL}PIPERIDIN-4-YL)ETHYL]PIPERIDIN-4-YL}-3-[6-(2-HYDROXYPHENYL)PYRIDAZIN-4-YL]-N-METHYL-3-AZABICYCLO[3.2.1]OCTANE-8-CARBOXAMIDE

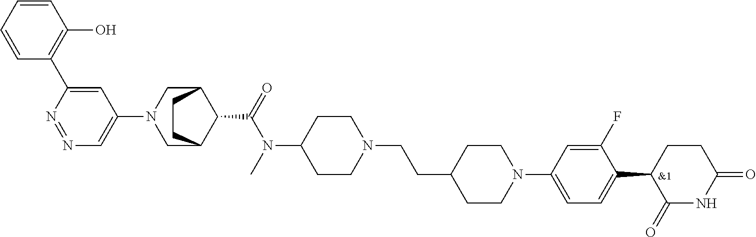 [SMILES: CN(C(=O)[C@H]1[C@@H]2CC[C@H]1CN(c1cnnc(-c3ccccc3O)c1)C2)C1CCN(CCC2CCN(c3ccc([C@H]4CCC(=O)NC4=O)c(F)c3)CC2)CC1]

The title compound was synthesized as described for Example 153 step 5, using (1R,5S,8S)-3-[6-(2-hydroxyphenyl)pyridazin-4-yl]-N-methyl-N-(piperidin-4-yl)-3-azabicyclo[3.2.1]octane-8-carboxamide hydrochloride and rac-2-(1-{4-[(3R)-2,6-dioxopiperidin-3-yl]-3-fluorophenyl}piperidin-4-yl)acetaldehyde to yield (1R,5S,8R)—N-{1-[2-(1-{4-[(3RS)-2,6-dioxopiperidin-3-yl]-3-fluorophenyl}piperidin-4-yl)ethyl]piperidin-4-yl}-3-[6-(2-hydroxyphenyl)pyridazin-4-yl]-N-methyl-3-azabicyclo[3.2.1]octane-8-carboxamide (0.0118 g, 67.70%). LCMS: C₄₂H₅₂FN₇O₄ requires: 737.4, found: m/z=738.2 [M+H]⁺.

### Example 159

### (S)—N-(1-(2-(1-(4-((S)-2,6-DIOXOPIPERIDIN-3-YL)-3,5-DIFLUOROPHENYL)PIPERIDIN-4-YL)ETHYL)PIPERIDIN-4-YL)-3-FLUORO-1-(6-(2-HYDROXYPHENYL)PYRIDAZIN-4-YL)-N-METHYLPYRROLIDINE-3-CARBOXAMIDE

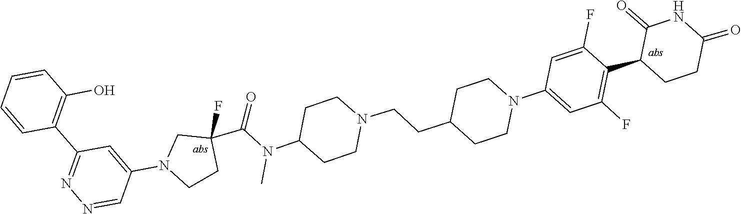 [SMILES: CN(C(=O)[C@]1(F)CCN(c2cnnc(-c3ccccc3O)c2)C1)C1CCN(CCC2CCN(c3cc(F)c([C@@H]4CCC(=O)NC4=O)c(F)c3)CC2)CC1]

### Step 1: Synthesis of the Title Compound

The title compound was prepared as described for the synthesis of Example 1, Step 2, using rac-(R)-3-fluoro-1-(6-(2-hydroxyphenyl)pyridazin-4-yl)-N-methyl-N-(piperidin-4-yl)pyrrolidine-3-carboxamide hydrochloride (Example 144, Step 1) and rac-(R)-2-(1-(4-(2,6-dioxopiperidin-3-yl)-3,5-difluorophenyl)piperidin-4-yl)acetaldehyde. After RP-HPLC, the compound was subjected to two stages of SFC purification (Chiralpak IH, EtOH+60ACN+0.1% NH₄OH followed by Chiralpak IBN, EtOH+60ACN+0.1% NH₄OH) to afford the title compound (Peak 1 after Stage 2 SFC, RT=2.98 min, 10.9 mgs, 9% yield) as a white solid. Assignment of absolute stereochemistry is arbitrary. LCMS C₃₉H₄₆F₃N₇O₄ requires 733.4, found m/z=734.4 [M+H]⁺.

### Example 160

### (R)—N-(1-(2-(1-(4-((S)-2,6-DIOXOPIPERIDIN-3-YL)-3,5-DIFLUOROPHENYL)PIPERIDIN-4-YL)ETHYL)PIPERIDIN-4-YL)-3-FLUORO-1-(6-(2-HYDROXYPHENYL)PYRIDAZIN-4-YL)-N-METHYLPYRROLIDINE-3-CARBOXAMIDE

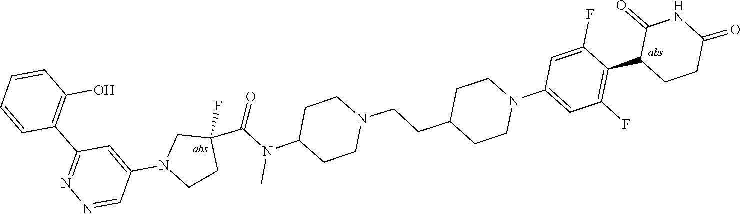

### Step 1: Synthesis of the Title Compound

The title compound was prepared as described for the synthesis of Example 1, Step 2, using rac-(R)-3-fluoro-1-(6-(2-hydroxyphenyl)pyridazin-4-yl)-N-methyl-N-(piperidin-4-yl)pyrrolidine-3-carboxamide hydrochloride (Example 144, Step 1) and rac-(R)-2-(1-(4-(2,6-dioxopiperidin-3-yl)-3,5-difluorophenyl)piperidin-4-yl)acetaldehyde. After RP-HPLC, the compound was subjected to SFC purification (Chiralpak IH, EtOH+60ACN+0.1% NH₄OH to afford the title compound (third peak after SFC, RT=6.37 min, 11.5 mgs, 9% yield) as a white solid. Assignment of absolute stereochemistry is arbitrary. LCMS C₃₉H₄₆F₃N₇O₄ requires 733.4, found m/z=734.4 [M+H]⁺.

### Example 161

### (RS)—N-CYCLOPROPYL-N-(1-(2-(1-(4-((R)-2,6-DIOXOPIPERIDIN-3-YL)PHENYL)PIPERIDIN-4-YL)ETHYL)PIPERIDIN-4-YL)-3-FLUORO-1-(6-(2-HYDROXYPHENYL)PYRIDAZIN-4-YL)PYRROLIDINE-3-CARBOXAMIDE

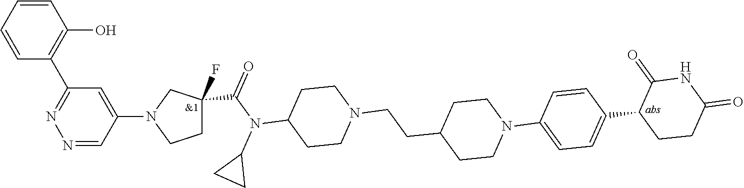

### Step 1: Synthesis of rac-(R)—N-cyclopropyl-3-fluoro-1-(6-(2-hydroxyphenyl)pyridazin-4-yl)-N-(piperidin-4-yl)pyrrolidine-3-carboxamide hydrochloride

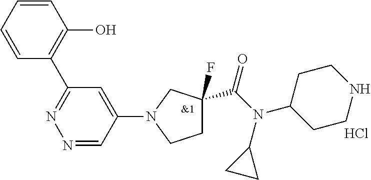 [SMILES: O=C(N(C1CCNCC1)C1CC1)[C@]1(F)CCN(c2cnnc(-c3ccccc3O)c2)C1 ; Cl]

The title compound was prepared as described for the synthesis of Example 1, Step 1, using rac-(R)-3-fluoro-1-(6-(2-(methoxymethoxy)phenyl)pyridazin-4-yl)pyrrolidine-3-carboxylic acid (Intermediate 28) and tert-butyl 4-(cyclopropylamino)piperidine-1-carboxylate to afford the title compound as a yellow oil. LCMS C₂₃H₂₈FN₅O₂ requires 425.2, found m/z=426.2 [M+H]⁺.

### Step 2: Synthesis of the Title Compound

The title compound was prepared as described for the synthesis of Example 2, Step 2, using Synthesis of rac-(R)—N-cyclopropyl-3-fluoro-1-(6-(2-hydroxyphenyl)pyridazin-4-yl)-N-(piperidin-4-yl)pyrrolidine-3-carboxamide hydrochloride (Example 161, Step 1) to afford the title compound (6.5 mgs, 7% yield), as a white solid. LCMS: C₄₁H₅₀FN₇O₄ requires: 723.4, found: m/z=724.4 [M+H]⁺.

### Example 162

### (1R,5S,8R)—N-{1-[2-(1-{4-[(3RS)-2,6-DIOXOPIPERIDIN-3-YL]-3-FLUOROPHENYL}PIPERIDIN-4-YL)ETHYL]PIPERIDIN-4-YL}-3-[6-(2-HYDROXYPHENYL)PYRIDAZIN-4-YL]-N-(2-METHOXYETHYL)-3-AZABICYCLO[3.2.1]OCTANE-8-CARBOXAMIDE

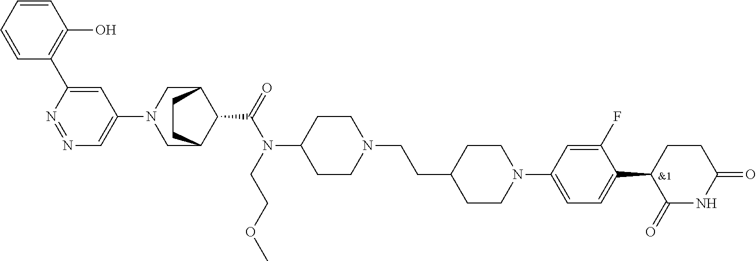 [SMILES: COCCN(C(=O)[C@H]1[C@@H]2CC[C@H]1CN(c1cnnc(-c3ccccc3O)c1)C2)C1CCN(CCC2CCN(c3ccc([C@H]4CCC(=O)NC4=O)c(F)c3)CC2)CC1]

### Step 1: tert-butyl 4-[N-(2-methoxyethyl)(1R,5S,8S)-3-{6-[2-(methoxymethoxy)phenyl]pyridazin-4-yl}-3-azabicyclo[3.2.1]octane-8-amido]piperidine-1-carboxylate

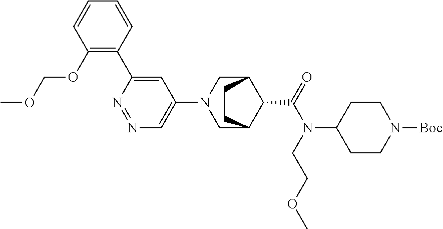 [SMILES: COCCN(C(=O)[C@H]1[C@@H]2CC[C@H]1CN(c1cnnc(-c3ccccc3OCOC)c1)C2)C1CCN(C(=O)OC(C)(C)C)CC1]

tert-butyl 4-[N-(2-methoxyethyl)(1R,5S,8S)-3-{6-[2-(methoxymethoxy)phenyl]pyridazin-4-yl}-3-azabicyclo[3.2.1]octane-8-amido]piperidine-1-carboxylate was synthesized as described for Example 153 step 3, using (1R,5S,8S)-3-{6-[2-(methoxymethoxy)phenyl]pyridazin-4-yl}-3-azabicyclo[3.2.1]octane-8-carboxylic acid and tert-butyl 4-[(2-methoxyethyl)amino]piperidine-1-carboxylate to yield tert-butyl 4-[N-(2-methoxyethyl)(1R,5S,8S)-3-{6-[2-(methoxymethoxy)phenyl]pyridazin-4-yl}-3-azabicyclo[3.2.1]octane-8-amido]piperidine-1-carboxylate (0.0253 g, 32.28%). LCMS: C₃₃H₄₇N₅O₆ requires: 609.4, found: m/z=610.2 [M+H]⁺.

### Step 2: (1R,5S,8S)-3-[6-(2-hydroxyphenyl)pyridazin-4-yl]-N-(2-methoxyethyl)-N-(piperidin-4-yl)-3-azabicyclo[3.2.1]octane-8-carboxamide

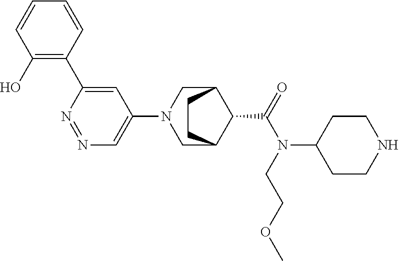 [SMILES: COCCN(C(=O)[C@H]1[C@@H]2CC[C@H]1CN(c1cnnc(-c3ccccc3O)c1)C2)C1CCNCC1]

(1R,5S,8S)-3-[6-(2-hydroxyphenyl)pyridazin-4-yl]-N-(2-methoxyethyl)-N-(piperidin-4-yl)-3-azabicyclo[3.2.1]octane-8-carboxamide was prepared as described for Example 107 step 2. LCMS: C₂₆H₃₅N₅O₃ requires: 465.3, found: m/z=466.2 [M+H]⁺.

### Step 3: Synthesis of the Title Compound

The title compound was synthesized as described for Example 153 step 5, using (1R,5S,8S)-3-[6-(2-hydroxyphenyl)pyridazin-4-yl]-N-(2-methoxyethyl)-N-(piperidin-4-yl)-3-azabicyclo[3.2.1]octane-8-carboxamide hydrochloride and rac-2-(1-{4-[(3R)-2,6-dioxopiperidin-3-yl]-3-fluorophenyl}piperidin-4-yl)acetaldehyde to yield (1R,5S,8R)—N-{1-[2-(1-{4-[(3RS)-2,6-dioxopiperidin-3-yl]-3-fluorophenyl}piperidin-4-yl)ethyl]piperidin-4-yl}-3-[6-(2-hydroxyphenyl)pyridazin-4-yl]-N-(2-methoxyethyl)-3-azabicyclo[3.2.1]octane-8-carboxamide (0.0055 g, 36.50%). LCMS: C₄₄H₅₆FN₇O₅ requires: 781.4, found: m/z=782.2 [M+H]⁺.

### Example 163

### (1R,5S,6S)—N-{1-[2-(1-{1-[(3RS)-2,6-DIOXOPIPERIDIN-3-YL]-3-METHYL-2-OXO-2,3-DIHYDRO-1H-1,3-BENZODIAZOL-4-YL}PIPERIDIN-4-YL)ETHYL]PIPERIDIN-4-YL}-N-ETHYL-3-[6-(2-HYDROXYPHENYL)PYRIDAZIN-4-YL]-3-AZABICYCLO[3.1.1]HEPTANE-6-CARBOXAMIDE

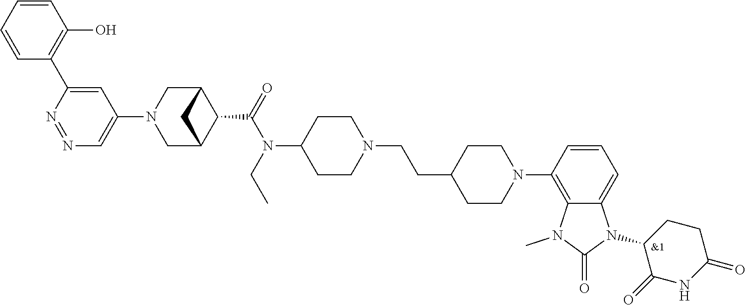 [SMILES: CCN(C(=O)[C@H]1[C@@H]2C[C@H]1CN(c1cnnc(-c3ccccc3O)c1)C2)C1CCN(CCC2CCN(c3cccc4c3n(C)c(=O)n4[C@@H]3CCC(=O)NC3=O)CC2)CC1]

### Step 1: methyl 3-{6-[2-(methoxymethoxy)phenyl]pyridazin-4-yl}-3-azabicyclo[3.1.1]heptane-6-carboxylate

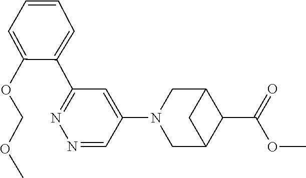 [SMILES: COCOc1ccccc1-c1cc(N2CC3CC(C2)C3C(=O)OC)cnn1]

Methyl 3-azabicyclo[3.1.1]heptane-6-carboxylate hydrochloride (481.67 mg, 2.5132 mmol), 5-chloro-3-[2-(methoxymethoxy)phenyl]pyridazine (420.00 mg, 1.6754 mmol) and N,N-diisopropylethylamine (875.52 μL, 0.65 g, 5.0263 mmol) were dissolved in DMSO (5.00 mL) and stirred in an 80° C. heating block overnight. Added more DIPEA (0.2 mL) and amine (53 μL) and heated to 100° C. Purified by RP-HPLC to yield methyl 3-{6-[2-(methoxymethoxy)phenyl]pyridazin-4-yl}-3-azabicyclo[3.1.1]heptane-6-carboxylate (0.589 g, 72.72%). C₂₀H₂₃N₃O₄ requires: 369.2, found: m/z=370.2 [M+H]⁺.

### Step 2: 3-{6-[2-(methoxymethoxy)phenyl]pyridazin-4-yl}-3-azabicyclo[3.1.1]heptane-6-carboxylic acid

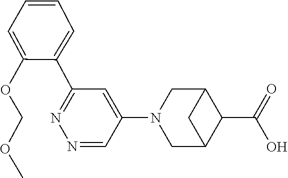 [SMILES: COCOc1ccccc1-c1cc(N2CC3CC(C2)C3C(=O)O)cnn1]

Dissolved methyl 3-{6-[2-(methoxymethoxy)phenyl]pyridazin-4-yl}-3-azabicyclo[3.1.1]heptane-6-carboxylate (589.00 mg, 1.5944 mmol) in THF (8.00 mL) and added 0.2 M LiOH (8.77 mL, 73.59 mg, 1.7538 mmol). Added more LiOH 0.2 M LiOH (8.77 mL, 73.59 mg, 1.7538 mmol). After another hour, the THF was removed and the solution was frozen and lyophilized (quantitative yield). LCMS: C₁₉H₂₁N₃O₄ requires: 355.2, found: m/z=356.2 [M+H]⁺.

### Step 3: tert-butyl 4-((1R,5S,6S)—N-ethyl-3-(6-(2-(methoxymethoxy)phenyl)pyridazin-4-yl)-3-azabicyclo[3.1.1]heptane-6-carboxamido)piperidine-1-carboxylate

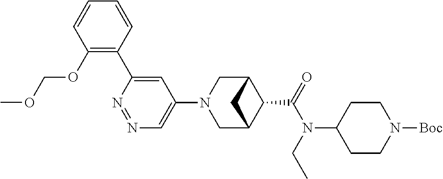 [SMILES: CCN(C(=O)[C@H]1[C@@H]2C[C@H]1CN(c1cnnc(-c3ccccc3OCOC)c1)C2)C1CCN(C(=O)OC(C)(C)C)CC1]

To a solution of 3-{6-[2-(methoxymethoxy)phenyl]pyridazin-4-yl}-3-azabicyclo[3.1.1]heptane-6-carboxylic acid (201.00 mg, 0.5656 mmol) in DMA (1.00 mL) was added tert-butyl 4-(ethylamino)piperidine-1-carboxylate (193.71 mg, 0.8484 mmol) and N,N-diisopropylethylamine (295.55 μL, 0.22 g, 1.6967 mmol). [(dimethylamino)({[1,2,3]triazolo[4,5-b]pyridin-3-yloxy})methylidene]dimethylazanium; hexafluoro-lambda5-phosphanuide (258.06 mg, 0.6787 mmol) was added and the reaction was stirred at rt for 2h. Purified directly by RP-HPLC followed by chiral SFC separation to yield the later eluting peak tert-butyl 4-((1R,5S,6S)—N-ethyl-3-(6-(2-(methoxymethoxy)phenyl)pyridazin-4-yl)-3-azabicyclo[3.1.1]heptane-6-carboxamido)piperidine-1-carboxylate (0.052 g, 13.53%). LCMS: C₃₁H₄₃N₅O₅ requires: 565.3, found: m/z=566.4 [M+H]⁺. The later eluting peak was carried forward to the next step and stereochemistry was assigned arbitrarily.

### Step 4: (1R,5S,6S)—N-ethyl-3-[6-(2-hydroxyphenyl)pyridazin-4-yl]-N-(piperidin-4-yl)-3-azabicyclo[3.1.1]heptane-6-carboxamide

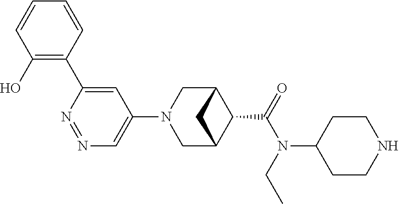 [SMILES: CCN(C(=O)[C@H]1[C@@H]2C[C@H]1CN(c1cnnc(-c3ccccc3O)c1)C2)C1CCNCC1]

(1R,5S,6S)—N-ethyl-3-[6-(2-hydroxyphenyl)pyridazin-4-yl]-N-(piperidin-4-yl)-3-azabicyclo[3.1.1]heptane-6-carboxamide was prepared as described for Example 107 step 2. LCMS: C₂₄H₃₁N₅O₂ requires: 421.2, found: m/z=422.2 [M+H]⁺.

### Step 5: Synthesis of the Title Compound

The title compound was synthesized as described for Example 153 step 5, using (1R,5S,6S)—N-ethyl-3-[6-(2-hydroxyphenyl)pyridazin-4-yl]-N-(piperidin-4-yl)-3-azabicyclo[3.1.1]heptane-6-carboxamide hydrochloride and rac-2-(1-{1-[(3R)-2,6-dioxopiperidin-3-yl]-3-methyl-2-oxo-1,3-benzodiazol-4-yl}piperidin-4-yl)acetaldehyde to yield (1R,5S,6S)—N-{1-[2-(1-{1-[(3RS)-2,6-dioxopiperidin-3-yl]-3-methyl-2-oxo-1,3-benzodiazol-4-yl}piperidin-4-yl)ethyl]piperidin-4-yl}-N-ethyl-3-[6-(2-hydroxyphenyl)pyridazin-4-yl]-3-azabicyclo[3.1.1]heptane-6-carboxamide (0.0041 g, 65.87%). LCMS: C₄₄H₅₅N₉O₅ requires: 789.4, found: m/z=790.2 [M+H]⁺.

### Example 164

### RAC-(3R)-3-(2,6-DIFLUORO-4-(4-(2-(4-(3-(6-(2-HYDROXYPHENYL)PYRIDAZIN-4-YL)-3,8-DIAZABICYCLO[3.2.1]OCTANE-8-CARBONYL)PIPERIDIN-1-YL)ETHYL)PIPERIDIN-1-YL)PHENYL)PIPERIDINE-2,6-DIONE

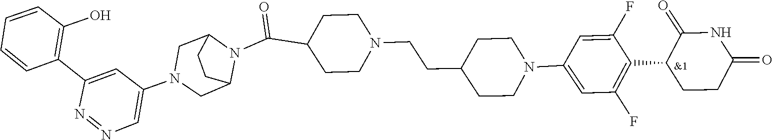 [SMILES: O=C1CC[C@H](c2c(F)cc(N3CCC(CCN4CCC(C(=O)N5C6CCC5CN(c5cnnc(-c7ccccc7O)c5)C6)CC4)CC3)cc2F)C(=O)N1]

### Step 1: Synthesis of 3-(6-(2-(methoxymethoxy)phenyl)pyridazin-4-yl)-3,8-diazabicyclo[3.2.1]octane

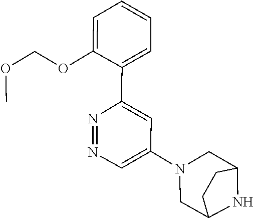 [SMILES: COCOc1ccccc1-c1cc(N2CC3CCC(C2)N3)cnn1]

tert-butyl 3,8-diazabicyclo[3.2.1]octane-8-carboxylate (400.00 mg, 1.8842 mmol) and 5-chloro-3-[2-(methoxymethoxy)phenyl]pyridazine (472.33 mg, 1.8842 mmol) were dissolved in 6 mL DMSO under argon atmosphere. N,N-diisopropylethylamine (1.65 mL, 1217.63 mg, 9.4209 mmol) was then added and the resulting solution was stirred at 100° C. overnight. The reaction was monitored via LCMS. Once complete, the reaction was concentrated to remove excess DIPEA and then quenched with H₂O and extracted with DCM. The organic phases were combined, dried over sodium sulfate and concentrated in vacuo. The resulting residue was purified on silica gel (MeOH:DCM gradient) to afford the desired BOC-protected product (267 mg) as a yellow foam. The product was then dissolved in 2 mL MeOH and 4M HCl in dioxane (2 mL) was added in one portion. The reaction was monitored via LCMS and once complete, the mixture was concentrated in vacuo to afford the desired compound (220 mg, 35.5%) as a red oil.

### Step 2: Synthesis of (3-(6-(2-(methoxymethoxy)phenyl)pyridazin-4-yl)-3,8-diazabicyclo[3.2.1]octan-8-yl)(piperidin-4-yl)methanone

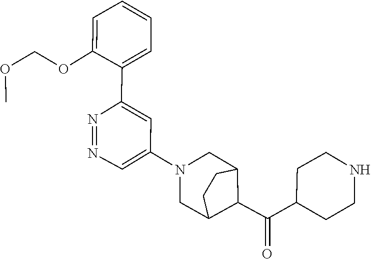 [SMILES: COCOc1ccccc1-c1cc(N2CC3CCC(C2)C3C(=O)C2CCNCC2)cnn1]

2-(5-{3,8-diazabicyclo[3.2.1]octan-3-yl}pyridazin-3-yl)phenol (220.00 mg, 0.7792 mmol) was dissolved in DMA (2.00 mL) and 1-(tert-butoxycarbonyl)piperidine-4-carboxylic acid (178.65 mg, 0.7792 mmol) and N,N-diisopropylethylamine (0.41 mL, 302.12 mg, 2.3375 mmol) were added sequentially. [(dimethylamino)({[1,2,3]triazolo[4,5-b]pyridin-3-yloxy})methylidene]dimethylazanium; hexafluoro-lambda5-phosphanuide (444.41 mg, 1.1688 mmol) was then added and the reaction was stirred at RT and monitored via LCMS. Once complete, the reaction was diluted with water and extracted with EtOAc 3×. The organic phases were dried over sodium sulfate, filtered and concentrated in vacuo. The resulting residue was purified on silica gel to afford the desired product (172 mg) as an orange oil. The material was then dissolved in 2.0 mL MeOH and 4M HCl in dioxane (2.0 mL) was added in one portion. The resulting solution was stirred at RT for 1 hour and monitored via LCMS. Once complete, the reaction was concentrated in vacuo to afford the desired product (240 mg) as a brown solid.

### Step 3: Synthesis of the Title Compound

2-{5-[8-(piperidine-4-carbonyl)-3,8-diazabicyclo[3.2.1]octan-3-yl]pyridazin-3-yl}phenol (8.00 mg, 0.0203 mmol) was dissolved in DMA (1.00 mL) and rac-2-(1-{4-[(3R)-2,6-dioxopiperidin-3-yl]-3,5-difluorophenyl}piperidin-4-yl)acetaldehyde (6.11 mg, 0.0203 mmol) was added in one portion. N,N-diisopropylethylamine (0.02 mL, 13.14 mg, 0.1017 mmol) was then added followed by sodium triacetoxyborohydride (12.93 mg, 0.0610 mmol). The resulting reaction was stirred at RT for 1 hour and monitored via LCMS. Once complete, the reaction was concentrated in vacuo and purified via RP—FC to afford the title compound (2.3 mg, 14.3%) as a trifluoroacetate salt. LCMS: C₄₀H₄₇F₂N₇O₄ requires: 727.4, found: m/z=728.1 [M+H]⁺.

### Example 165

### (R)—N-(1-(2-(1-(4-(2,6-DIOXOPIPERIDIN-3-YL)PHENYL)PIPERIDIN-4-YL)ETHYL)PIPERIDIN-4-YL)-N-ETHYL-4-FLUORO-1-(6-(2-HYDROXYPHENYL)PYRIDAZIN-4-YL)PIPERIDINE-4-CARBOXAMIDE

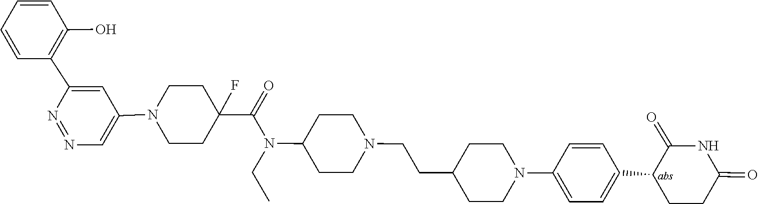 [SMILES: CCN(C(=O)C1(F)CCN(c2cnnc(-c3ccccc3O)c2)CC1)C1CCN(CCC2CCN(c3ccc([C@H]4CCC(=O)NC4=O)cc3)CC2)CC1]

### Step 1: Synthesis of benzyl 4-(N-ethyl-4-fluoropiperidine-4-carboxamido)piperidine-1-carboxylate

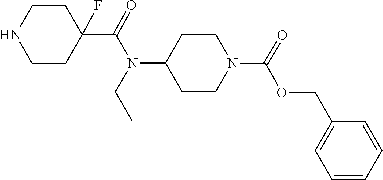 [SMILES: CCN(C(=O)C1(F)CCNCC1)C1CCN(C(=O)OCc2ccccc2)CC1]

The title compound was prepared as described for Example 92, Step 1, using 1-(tert-butoxycarbonyl)-4-fluoropiperidine-4-carboxylic acid and benzyl 4-(ethylamino)piperidine-1-carboxylate to afford the desired product as a colorless oil. LCMS: C₂₁H₃₀FN₃O₃ requires 391.2, found: m/z=392.2 [M+H]⁺.

### Step 2: Synthesis of benzyl 4-(N-ethyl-4-fluoro-1-(6-(2-hydroxyphenyl)pyridazin-4-yl)piperidine-4-carboxamido)piperidine-1-carboxylate

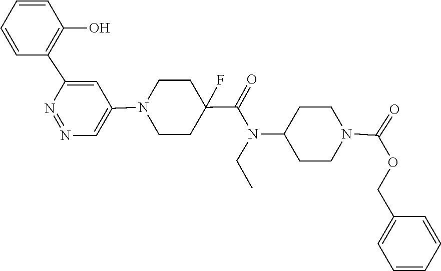 [SMILES: CCN(C(=O)C1(F)CCN(c2cnnc(-c3ccccc3O)c2)CC1)C1CCN(C(=O)OCc2ccccc2)CC1]

The title compound was prepared as described for Example 128, Step 3, using 5-chloro-3-(2-(methoxymethoxy)phenyl)pyridazine and benzyl 4-(N-ethyl-4-fluoropiperidine-4-carboxamido)piperidine-1-carboxylate to afford the desired product as a yellow foam. LCMS: C₃₁H₃₆FN₅O₄ requires 561.3, found: m/z=562.2 [M+H]⁺.

### Step 3: Synthesis of N-ethyl-4-fluoro-1-(6-(2-hydroxyphenyl)pyridazin-4-yl)-N-(piperidin-4-yl)piperidine-4-carboxamide

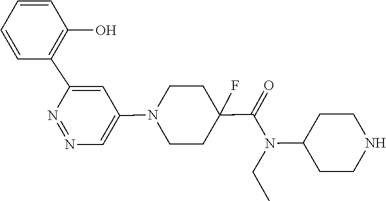 [SMILES: CCN(C(=O)C1(F)CCN(c2cnnc(-c3ccccc3O)c2)CC1)C1CCNCC1]

To a round bottom flask containing palladium (301.21 mg, 0.28 mmol) and EtOH (4.00 mL) was added benzyl 4-{N-ethyl-4-fluoro-1-[6-(2-hydroxyphenyl)pyridazin-4-yl]piperidine-4-amido}piperidine-1-carboxylate (310.00 mg, 0.57 mmol) dissolved in THF (4.00 mL). The flask was fitted with a three-way adapter and a hydrogen balloon. The atmosphere was changed to hydrogen, and the reaction was stirred at room temperature until complete by LCMS. Then, the crude product was filtered through a plug of celite, and the solvent was removed under reduced pressure to afford the desired product as a yellow oil (206 mg, 85%). LCMS: C₂₃H₃₀FN₅O₂ requires 427.2, found: m/z=428.2 [M+H]⁺.

### Step 4: Synthesis of the Title Compound

The title compound was prepared as described for Example 1, Step 2, using N-ethyl-4-fluoro-1-(6-(2-hydroxyphenyl)pyridazin-4-yl)-N-(piperidin-4-yl)piperidine-4-carboxamide and (R)-2-(1-(4-(2,6-dioxopiperidin-3-yl)phenyl)piperidin-4-yl)acetaldehyde to afford the title compound (19.7 mg, 53%). LCMS: C₄₁H₅₂FN₇O₄ requires 725.4, found: m/z=726.4 [M+H]⁺.

### Example 166

### RAC-(1R,5S,8S)—N-(1-(2-(1-(1-((R)-2,6-DIOXOPIPERIDIN-3-YL)-3-METHYL-2-oxo-2,3-DIHYDRO-1H-BENZO[D]IMIDAZOL-4-YL)PIPERIDIN-4-YL)ETHYL)PIPERIDIN-4-YL)-3-(6-(2-HYDROXYPHENYL)PYRIDAZIN-4-YL)-N-(2,2,2-TRIFLUOROETHYL)-3-AZABICYCLO[3.2.1]OCTANE-8-CARBOXAMIDE

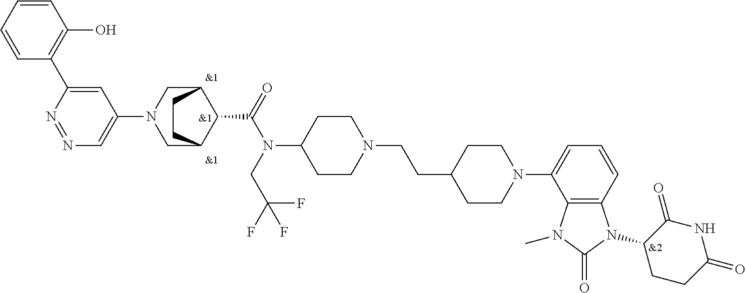 [SMILES: Cn1c(=O)n([C@H]2CCC(=O)NC2=O)c2cccc(N3CCC(CCN4CCC(N(CC(F)(F)F)C(=O)[C@H]5[C@@H]6CC[C@H]5CN(c5cnnc(-c7ccccc7O)c5)C6)CC4)CC3)c21]

### Step 1: Synthesis of (1R,5S,8s)-3-(6-(2-hydroxyphenyl)pyridazin-4-yl)-N-(piperidin-4-yl)-N-(2,2,2-trifluoroethyl)-3-azabicyclo[3.2.1]octane-8-carboxamide

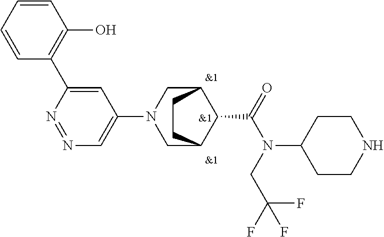 [SMILES: O=C([C@H]1[C@@H]2CC[C@H]1CN(c1cnnc(-c3ccccc3O)c1)C2)N(CC(F)(F)F)C1CCNCC1]

The title compound was prepared as described for Example 89, Step 1, using tert-butyl (R)-4-(sec-butylamino)piperidine-1-carboxylate to afford the desired product as a dark yellow solid. LCMS: C₂₅H₃₀F₃N₅O₂ requires 489.2, found: m/z=490.2 [M+H]⁺.

### Step 3: Synthesis of the Title Compound

The title compound was prepared as described for Example 1, Step 2, using (1R,5S,8s)-3-(6-(2-hydroxyphenyl)pyridazin-4-yl)-N-(piperidin-4-yl)-N-(2,2,2-trifluoroethyl)-3-azabicyclo[3.2.1]octane-8-carboxamide and rac-(R)-2-(1-(1-(2,6-dioxopiperidin-3-yl)-3-methyl-2-oxo-2,3-dihydro-1H-benzo[d]imidazol-4-yl)piperidin-4-yl)acetaldehyde to afford the title compound (7.4 mg, 28%). LCMS: C₄₅H₅₄F₃N₉O₅ requires 857.4, found: m/z=858.3 [M+H]⁺.

### Example 167A

### (3RS)-3-CYCLOBUTOXY-N-{1-[2-(1-{4-[(3R)-2,6-DIOXOPIPERIDIN-3-YL]PHENYL}PIPERIDIN-4-YL)ETHYL]PIPERIDIN-4-YL}-1-[6-(2-HYDROXYPHENYL)PYRIDAZIN-4-YL]-N-METHYLPYRROLIDINE-3-CARBOXAMIDE

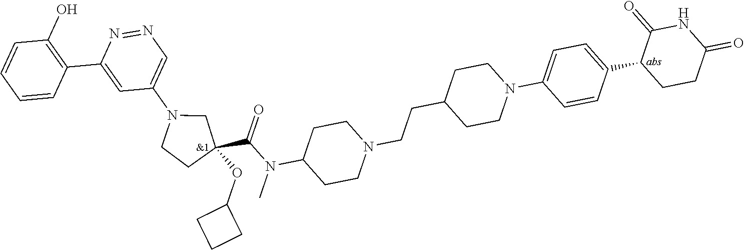 [SMILES: CN(C(=O)[C@@]1(OC2CCC2)CCN(c2cnnc(-c3ccccc3O)c2)C1)C1CCN(CCC2CCN(c3ccc([C@H]4CCC(=O)NC4=O)cc3)CC2)CC1]

### Step-1: Synthesis of tert-butyl 4-(3-cyclobutoxy-1-(6-(2-(methoxymethoxy)phenyl)pyridazin-4-yl)-N-methylpyrrolidine-3-carboxamido)piperidine-1-carboxylate

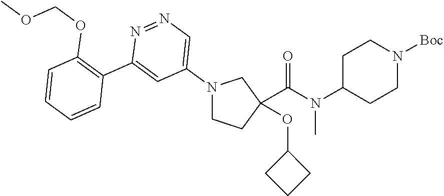 [SMILES: COCOc1ccccc1-c1cc(N2CCC(OC3CCC3)(C(=O)N(C)C3CCN(C(=O)OC(C)(C)C)CC3)C2)cnn1]

To an ice-cold stirred solution of 3-cyclobutoxy-1-(6-(2-(methoxymethoxy)phenyl)pyridazin-4-yl)pyrrolidine-3-carboxylic acid (250 mg, 0.626 mmol) in DMF (2 mL) was added DIPEA (0.32 mL, 1.88 mmol) under inert atmosphere. Then tert-butyl 4-(methylamino)piperidine-1-carboxylate 2 (201 mg, 0.939 mmol) and HATU (262 mg, 0.688 mmol) were added to the reaction mixture. The resulting reaction mixture was stirred at RT for 6 h. After completion of reaction (monitored by TLC and LCMS), the reaction mixture was quenched with ice cold water (30 mL) and extracted with EtOAc (3×50 mL). The combined organic layer was separated, dried over anhydrous Na₂SO₄, filtered and concentrated under reduced pressure to dryness. The crude obtained was purified by flash column chromatography (5-8% MeOH:DCM) to get tert-butyl 4-(3-cyclobutoxy-1-(6-(2-(methoxymethoxy)phenyl)pyridazin-4-yl)-N-methylpyrrolidine-3-carboxamido)piperidine-1-carboxylate (220 mg, 59%) as yellow solid. LCMS: m/z 596.31 [M+H]³⁰; Purity: 97%.

### Step-2: Synthesis of 3-cyclobutoxy-1-(6-(2-hydroxyphenyl)pyridazin-4-yl)-N-methyl-N-(piperidin-4-yl)pyrrolidine-3-carboxamide

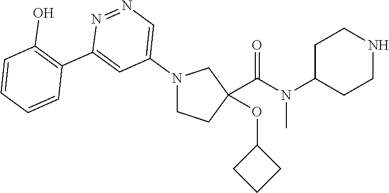 [SMILES: CN(C(=O)C1(OC2CCC2)CCN(c2cnnc(-c3ccccc3O)c2)C1)C1CCNCC1]

To an ice-cold stirred solution of tert-butyl 4-(3-cyclobutoxy-1-(6-(2-(methoxymethoxy)phenyl)pyridazin-4-yl)-N-methylpyrrolidine-3-carboxamido)piperidine-1-carboxylate (180 mg, 0.302 mmol) in DCM (4 mL) was added TFA (1 mL) and the reaction was stirred at room temperature for 6 h. After completion of reaction (monitored by TLC and LCMS), the reaction mixture was concentrated under reduced pressure to get crude. The crude compound was triturated with Diethyl ether: Pentane (1:1, 10 mL) to get 3-cyclobutoxy-1-(6-(2-hydroxyphenyl)pyridazin-4-yl)-N-methyl-N-(piperidin-4-yl)pyrrolidine-3-carboxamide as an off-white solid (130 mg, crude). LCMS: m/z 452.33 [M+H]⁺; Purity: 99%.

### Step-3: Synthesis of the Title Compound

To an ice-cold stirred solution of 3-cyclobutoxy-1-(6-(2-hydroxyphenyl)pyridazin-4-yl)-N-methyl-N-(piperidin-4-yl)pyrrolidine-3-carboxamide (130 mg, 0.288 mmol) in ACN:MeOH (4 mL, 3:1) were added (R)-2-(1-(4-(2,6-dioxopiperidin-3-yl)phenyl)piperidin-4-yl)acetaldehyde 5 (72.4 mg, 0.230 mmol) and DIPEA (0.24 mL, 1.44 mmol) and the reaction mixture was stirred for 30 min. Afterwards, STAB (183 mg, 0.864 mmol) was added to the reaction mixture at 0° C. and gradually warmed to RT for 2 h. After completion, the reaction was quenched with water (10 mL) and extracted with 10% MeOH in DCM (2×20 mL). The combined organic layer was dried over anhydrous Na₂SO₄, filtered and concentrated under reduced pressure. The crude product was purified by RP-HPLC purification using Column: X Select fluorophenyl (250×19), 5 μm Mobile Phase A: 0.1% Formic Acid in Water Mobile Phase B: Acetonitrile with flow rate 18 mL/min to get 3-cyclobutoxy-N-(1-(2-(1-(4-((R)-2,6-dioxopiperidin-3-yl)phenyl)piperidin-4-yl)ethyl)piperidin-4-yl)-1-(6-(2-hydroxyphenyl)pyridazin-4-yl)-N-methylpyrrolidine-3-carboxamide as an off white solid (59 mg, 26% over 2 steps). LCMS: m/z 750.59 [M+H]⁺; Purity: 98.57%. ¹H NMR (400 MHz, DMSO-d6): δ 14.76 (brs, 1H), 10.76 (s, 1H), 8.64 (s, 1H), 8.09 (d, J=7.4 Hz, 1H), 7.34 (t, J=8 Hz, 1H), 7.25 (brs, 1H),), 7.02 (d, J=8.6 Hz, 2H), 6.94-6.86 (m, 4H), 4.37-4.22 (m, 1H), 3.92-3.79 (m, 3H), 3.73-3.62 (m, 4H), 3.56-3.51 (m, 1H), 3.07-2.76 (m, 5H), 2.63-2.58 (m, 3H), 2.45-2.42 (m, 4H), 2.16-1.80 (m, 9H), 1.75-1.72 (m, 3H), 1.64-1.50 (m, 3H), 1.40 (brs, 4H) and 1.29-1.19 (m, 2H).

### Example 167B

### (S)-3-CYCLOBUTOXY-N-(1-(2-(1-(4-((R)-2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-N-METHYLPYRROLIDINE-3-CARBOXAMIDE

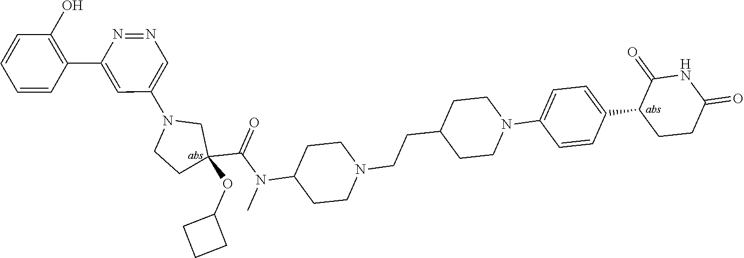 [SMILES: CN(C(=O)[C@]1(OC2CCC2)CCN(c2cnnc(-c3ccccc3O)c2)C1)C1CCN(CCC2CCN(c3ccc([C@H]4CCC(=O)NC4=O)cc3)CC2)CC1]

### Step 1: SFC Separation of Title Compound

The racemic compound was separated using SFC conditions (Stage 1 column: IBN, Stage 2 column: IH, EtOH+60% CAN+0.1% NH₄OH). Peak 1 was collected to yield the title compound (15.5 mg) as a white solid.

### Example 168

### (1R,5S,8R)—N-{1-[2-(1-{4-[(3R)-2,6-DIOXOPIPERIDIN-3-YL]PHENYL}PIPERIDIN-4-YL)ETHYL]PIPERIDIN-4-YL}-3-[6-(2-HYDROXYPHENYL)PYRIDAZIN-4-YL]-N-(2-METHOXYETHYL)-3-AZABICYCLO[3.2.1]OCTANE-8-CARBOXAMIDE

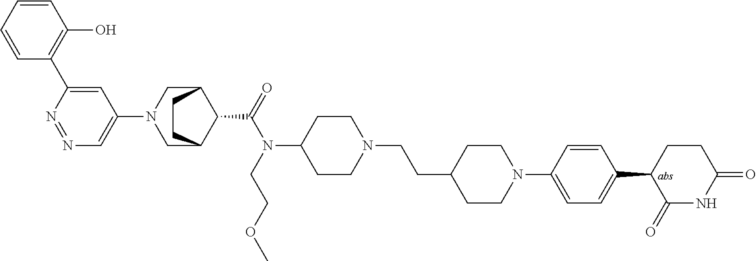 [SMILES: COCCN(C(=O)[C@H]1[C@@H]2CC[C@H]1CN(c1cnnc(-c3ccccc3O)c1)C2)C1CCN(CCC2CCN(c3ccc([C@H]4CCC(=O)NC4=O)cc3)CC2)CC1]

The title compound was synthesized as described for Example 153 step 5, using (1R,5S,8S)-3-[6-(2-hydroxyphenyl)pyridazin-4-yl]-N-(2-methoxyethyl)-N-(piperidin-4-yl)-3-azabicyclo[3.2.1]octane-8-carboxamide hydrochloride and 2-(1-{4-[(3R)-2,6-dioxopiperidin-3-yl]phenyl}piperidin-4-yl)acetaldehyde to yield (1R,5S,8R)—N-{1-[2-(1-{4-[(3R)-2,6-dioxopiperidin-3-yl]phenyl}piperidin-4-yl)ethyl]piperidin-4-yl}-3-[6-(2-hydroxyphenyl)pyridazin-4-yl]-N-(2-methoxyethyl)-3-azabicyclo[3.2.1]octane-8-carboxamide (0.0054 g, 39.43%). LCMS: C₄₄H₅₇N₇O₅ requires: 763.4, found: m/z=764.4 [M+H]⁺.

### Example 169

### (1R,5S,8R)—N-{1-[2-(1-{4-[(3R)-2,6-DIOXOPIPERIDIN-3-YL]-3-FLUOROPHENYL}PIPERIDIN-4-YL)ETHYL]PIPERIDIN-4-YL}-3-[6-(2-HYDROXYPHENYL)PYRIDAZIN-4-YL]-N-(2-METHOXYETHYL)-3-AZABICYCLO[3.2.1]OCTANE-8-CARBOXAMIDE

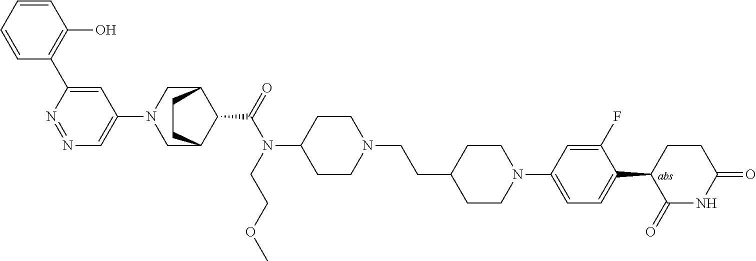 [SMILES: COCCN(C(=O)[C@H]1[C@@H]2CC[C@H]1CN(c1cnnc(-c3ccccc3O)c1)C2)C1CCN(CCC2CCN(c3ccc([C@H]4CCC(=O)NC4=O)c(F)c3)CC2)CC1]

Combined (1R,5S,8S)-3-[6-(2-hydroxyphenyl)pyridazin-4-yl]-N-(2-methoxyethyl)-N-(piperidin-4-yl)-3-azabicyclo[3.2.1]octane-8-carboxamide hydrochloride (19.00 mg, 0.0378 mmol) and 2-(1-{4-[(3R)-2,6-dioxopiperidin-3-yl]-3-fluorophenyl}piperidin-4-yl)acetaldehyde (13.21 mg, 0.0397 mmol) in ACN (0.40 mL) and methanol (0.20 mL) and then added sodium triacetoxyborohydride (12.03 mg, 0.0568 mmol) and stirred for 2h. Concentrated down to an oil, then redissolved in DCM and purified directly by MPLC (DCM/methanol) to yield (1R,5S,8R)—N-{1-[2-(1-{4-[(3R)-2,6-dioxopiperidin-3-yl]-3-fluorophenyl}piperidin-4-yl)ethyl]piperidin-4-yl}-3-[6-(2-hydroxyphenyl)pyridazin-4-yl]-N-(2-methoxyethyl)-3-azabicyclo[3.2.1]octane-8-carboxamide (0.0078 g, 23.46%). LCMS: C₄₄H₅₆FN₇O₅ requires: 781.4, found: m/z=782.2 [M+H]⁺.

### Example 170

### (1R,5S,8R)—N-{1-[2-(1-{4-[(3S)-2,6-DIOXOPIPERIDIN-3-YL]-3-FLUOROPHENYL}PIPERIDIN-4-YL)ETHYL]PIPERIDIN-4-YL}-3-[6-(2-HYDROXYPHENYL)PYRIDAZIN-4-YL]-N-(2-METHOXYETHYL)-3-AZABICYCLO[3.2.1]OCTANE-8-CARBOXAMIDE

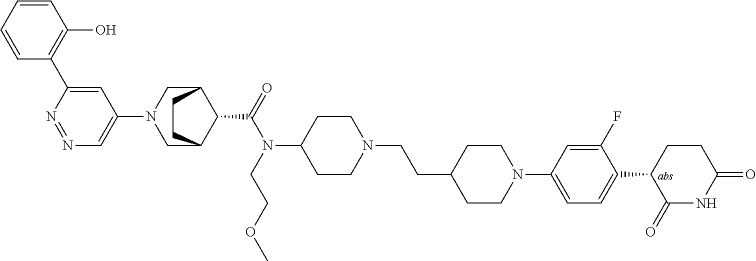 [SMILES: COCCN(C(=O)[C@H]1[C@@H]2CC[C@H]1CN(c1cnnc(-c3ccccc3O)c1)C2)C1CCN(CCC2CCN(c3ccc([C@@H]4CCC(=O)NC4=O)c(F)c3)CC2)CC1]

Combined (1R,5S,8S)-3-[6-(2-hydroxyphenyl)pyridazin-4-yl]-N-(2-methoxyethyl)-N-(piperidin-4-yl)-3-azabicyclo[3.2.1]octane-8-carboxamide hydrochloride (10.60 mg, 0.0211 mmol) and 2-(1-{4-[(3S)-2,6-dioxopiperidin-3-yl]-3-fluorophenyl}piperidin-4-yl)acetaldehyde (7.72 mg, 0.0232 mmol) in ACN (0.40 mL) and then added sodium triacetoxyborohydride (6.71 mg, 0.0317 mmol) and stirred for 2h. Concentrated down to an oil, then dissolved in DCM and purified by MPLC (DCM/methanol) to yield (1R,5S,8R)—N-{1-[2-(1-{4-[(3S)-2,6-dioxopiperidin-3-yl]-3-fluorophenyl}piperidin-4-yl)ethyl]piperidin-4-yl}-3-[6-(2-hydroxyphenyl)pyridazin-4-yl]-N-(2-methoxyethyl)-3-azabicyclo[3.2.1]octane-8-carboxamide (0.0058 g, 30.39%). LCMS: C₄₄H₅₆FN₇O₅ requires: 781.4, found: m/z=782.4 [M+H]⁺.

### Example 171

### (1R,5S,8R)—N-{1-[2-(1-{4-[(3R)-2,6-DIOXOPIPERIDIN-3-YL]PHENYL}PIPERIDIN-4-YL)ETHYL]PIPERIDIN-4-YL}-3-[6-(2-HYDROXYPHENYL)PYRIDAZIN-4-YL]-N-PROPYL-3-AZABICYCLO[3.2.1]OCTANE-8-CARBOXAMIDE

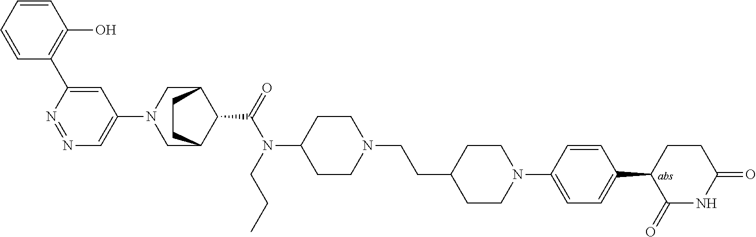 [SMILES: CCCN(C(=O)[C@H]1[C@@H]2CC[C@H]1CN(c1cnnc(-c3ccccc3O)c1)C2)C1CCN(CCC2CCN(c3ccc([C@H]4CCC(=O)NC4=O)cc3)CC2)CC1]

### Step 1: tert-butyl 4-[N-propyl(1R,5S,8S)-3-{6-[2-(methoxymethoxy)phenyl]pyridazin-4-yl}-3-azabicyclo[3.2.1]octane-8-amido]piperidine-1-carboxylate

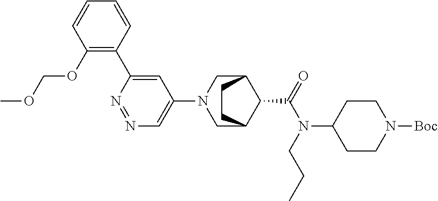 [SMILES: CCCN(C(=O)[C@H]1[C@@H]2CC[C@H]1CN(c1cnnc(-c3ccccc3OCOC)c1)C2)C1CCN(C(=O)OC(C)(C)C)CC1]

tert-butyl 4-[N-propyl(1R,5S,8S)-3-{6-[2-(methoxymethoxy)phenyl]pyridazin-4-yl}-3-azabicyclo[3.2.1]octane-8-amido]piperidine-1-carboxylate was synthesized as described for Example 153 step 3, using (1R,5S,8S)-3-{6-[2-(methoxymethoxy)phenyl]pyridazin-4-yl}-3-azabicyclo[3.2.1]octane-8-carboxylic acid and tert-butyl 4-(propylamino)piperidine-1-carboxylate to yield tert-butyl 4-[N-propyl(1R,5S,8S)-3-{6-[2-(methoxymethoxy)phenyl]pyridazin-4-yl}-3-azabicyclo[3.2.1]octane-8-amido]piperidine-1-carboxylate (0.045 g, 47.54%). LCMS: C₃₃H₄₇N₅O₅ requires: 593.4, found: m/z=594.2 [M+H]⁺.

### Step 2: (1R,5S,8S)-3-[6-(2-hydroxyphenyl)pyridazin-4-yl]-N-(piperidin-4-yl)-N-propyl-3-azabicyclo[3.2.1]octane-8-carboxamide

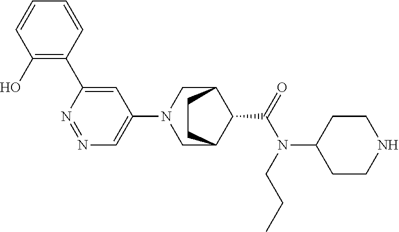 [SMILES: CCCN(C(=O)[C@H]1[C@@H]2CC[C@H]1CN(c1cnnc(-c3ccccc3O)c1)C2)C1CCNCC1]

(1R,5S,8S)-3-[6-(2-hydroxyphenyl)pyridazin-4-yl]-N-(piperidin-4-yl)-N-propyl-3-azabicyclo[3.2.1]octane-8-carboxamide was prepared as described for Example 107 step 2. LCMS: C₂₆H₃₅N₅O₂ requires: 449.3, found: m/z=450.2 [M+H]⁺.

### Step 3: Synthesis of the Title Compound

Combined (1R,5S,8S)-3-[6-(2-hydroxyphenyl)pyridazin-4-yl]-N-(piperidin-4-yl)-N-propyl-3-azabicyclo[3.2.1]octane-8-carboxamide hydrochloride (16.60 mg, 0.0342 mmol) and 2-(1-{4-[(3R)-2,6-dioxopiperidin-3-yl]phenyl}piperidin-4-yl)acetaldehyde (11.27 mg, 0.0359 mmol) in ACN (0.40 mL) and methanol (0.20 mL) and then added sodium triacetoxyborohydride (13.03 mg, 0.0615 mmol) and stirred for 2h. Concentrated down to an oil, then dissolved in DCM and purified on MPLC (DCM/methanol) to yield (1R,5S,8R)—N-{1-[2-(1-{4-[(3R)-2,6-dioxopiperidin-3-yl]phenyl}piperidin-4-yl)ethyl]piperidin-4-yl}-3-[6-(2-hydroxyphenyl)pyridazin-4-yl]-N-propyl-3-azabicyclo[3.2.1]octane-8-carboxamide (0.0149 g, 56.52%). LCMS: C₄₄H₅₇N₇O₄ requires: 747.4, found: m/z=748.4 [M+H]⁺.

### Example 172

### N-(1-(2-(1-(4-((R)-2,6-DIOXOPIPERIDIN-3-YL)PHENYL)PIPERIDIN-4-YL)ETHYL)PIPERIDIN-4-YL)-3-FLUORO-1-(6-(2-HYDROXYPHENYL)PYRIDAZIN-4-YL)-N-METHYLPIPERIDINE-3-CARBOXAMIDE

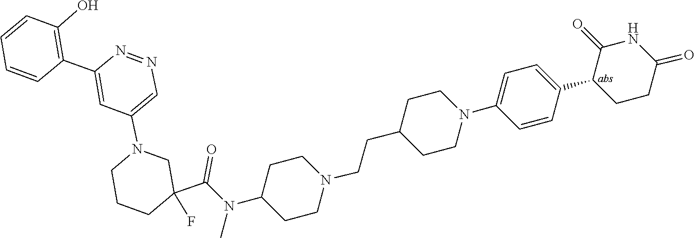 [SMILES: CN(C(=O)C1(F)CCCN(c2cnnc(-c3ccccc3O)c2)C1)C1CCN(CCC2CCN(c3ccc([C@H]4CCC(=O)NC4=O)cc3)CC2)CC1]

### Step-1: tert-butyl4-(3-fluoro-1-(6-(2-(methoxymethoxy)phenyl)pyridazin-4-yl)-N-methylpiperidine-3-carboxamido)piperidine-1-carboxylate

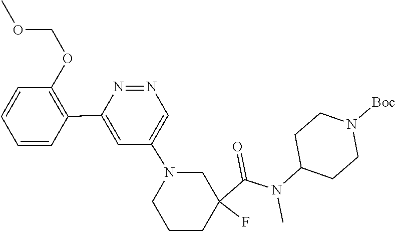 [SMILES: COCOc1ccccc1-c1cc(N2CCCC(F)(C(=O)N(C)C3CCN(C(=O)OC(C)(C)C)CC3)C2)cnn1]

To an ice cold stirred solution of 3-fluoro-1-(6-(2-(methoxymethoxy)phenyl)pyridazin-4-yl)piperidine-3-carboxylic acid Lithium salt (300 mg, 0.83 mmol) and tert-butyl 4-(methylamino)piperidine-1-carboxylate (267 mg, 1.25 mmol) in DMF (5 mL) was added DIPEA (0.5 ml, 2.49 mmol) followed by HATU (347 mg, 0.91 mmol) under inert atmosphere and reaction mixture was stirred at RT for 3 h. After completion, the reaction mixture was quenched with water (20 mL) and extracted with DCM (2×30 mL). The combined organic layer was washed with ice-cold water (3×20 mL), dried over anhydrous sodium sulphate, filtered and concentrated under reduced pressure to get the crude. The crude product was purified by flash column chromatography (−8% MeOH in DCM) to get tert-butyl 4-(3-fluoro-1-(6-(2-(methoxymethoxy)phenyl)pyridazin-4-yl)-N-methylpiperidine-3-carboxamido)piperidine-1-carboxylate as a yellow sticky solid (250 mg, 55%). LCMS: m/z 558.43 [M+H]⁺; Purity: 92%.

### Step-2: 3-fluoro-1-(6-(2-hydroxyphenyl)pyridazin-4-yl)-N-methyl-N-(piperidin-4-yl)piperidine-3-carboxamide HCl salt

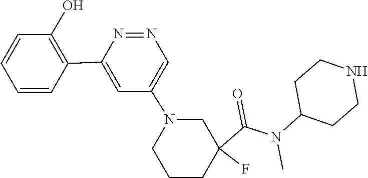 [SMILES: CN(C(=O)C1(F)CCCN(c2cnnc(-c3ccccc3O)c2)C1)C1CCNCC1]

To an ice-cold stirred solution of tert-butyl 4-(3-fluoro-1-(6-(2-(methoxymethoxy)phenyl)pyridazin-4-yl)-N-methylpiperidine-3-carboxamido)piperidine-1-carboxylate (230 mg, 0.41 mmol) in DCM (6 mL) was added 4 N HCl in Dioxane (2 mL) and the reaction mixture was stirred at RT for 5 h. After completion, the reaction mixture was concentrated under reduced pressure and triturated with Diethyl ether (30 mL) to get 3-fluoro-1-(6-(2-hydroxyphenyl)pyridazin-4-yl)-N-methyl-N-(piperidin-4-yl)piperidine-3-carboxamide HCl salt as an off white solid (170 mg, crude). LCMS: m/z 414.28 [M+H]⁺; Purity: 95%.

### Step-3: Synthesis of the Title Compound

To an ice cold stirred solution 3-fluoro-1-(6-(2-hydroxyphenyl)pyridazin-4-yl)-N-methyl-N-(piperidin-4-yl)piperidine-3-carboxamide HCl salt (170 mg, 0.41 mmol) and (R)-2-(1-(4-(2,6-dioxopiperidin-3-yl)phenyl)piperidin-4-yl)acetaldehyde (103 mg, 0.33 mmol) in CH₃CN:MeOH (3:1, 8 mL) was added DIPEA (0.35 mL, 2.06 mmol) and the reaction mixture was stirred at RT for 30 min before adding STAB (261 mg, 1.23 mmol) at 0° C. The reaction mixture was stirred at RT for another 3 h. After completion (monitored by TLC and LCMS), the reaction mixture was concentrated, quenched with water (20 mL) and extracted with 10% MeOH:DCM (2×30 mL). The combined organic layer was washed with brine (10 mL), dried over anhydrous sodium sulphate, filtered and concentrated under reduced pressure to get the crude. The crude product was purified by RP-HPLC purification Column: X-Bridge C8 250×19.5 μm, Mobile phase: A-5 mM Ammonium Acetate in Water B: —ACN with flow rate 18 ml/min to get N-(1-(2-(1-(4-((R)-2,6-dioxopiperidin-3-yl)phenyl)piperidin-4-yl)ethyl)piperidin-4-yl)-3-fluoro-1-(6-(2-hydroxyphenyl)pyridazin-4-yl)-N-methylpiperidine-3-carboxamide as an off white solid (74 mg, 25% over 2 steps). LCMS: m/z 712.63 M+H]⁺; Purity: 99.61%. 1H NMR (400 MHz, DMSO-d6): δ 14.58 (brs, 1H), 10.76 (s, 1H), 8.97 (s, 1H), 8.15-8.13 (m, 1H), 7.60 (s, 1H), 7.34-7.30 (m, 1H), 7.02 (d, J=8.0 Hz, 2H), 6.93-6.86 (m, 4H), 4.63-4.57 (m, 1H), 4.28-3.92 (m, 2H), 3.80-3.71 (m, 2H), 3.70-3.61 (m, 2H), 3.26-3.20 (m, 1H), 2.95-2.89 (m, 4H), 2.65-2.56 (m, 3H), 2.46-2.42 (m, 2H), 2.25-2.16 (m, 3H), 2.12-1.91 (m, 4H), 1.81-1.63 (m, 7H), 1.54-1.39 (in, 5H) and 1.23-1.19 (m, 2H).

### Example 173

### N-(1-(2-(1-(4-((R)-2,6-DIOXOPIPERIDIN-3-YL)PHENYL)PIPERIDIN-4-YL)ETHYL)PIPERIDIN-4-YL)-N-ETHYL-3-FLUORO-1-(6-(2-HYDROXYPHENYL)PYRIDAZIN-4-YL)PIPERIDINE-3-CARBOXAMIDE

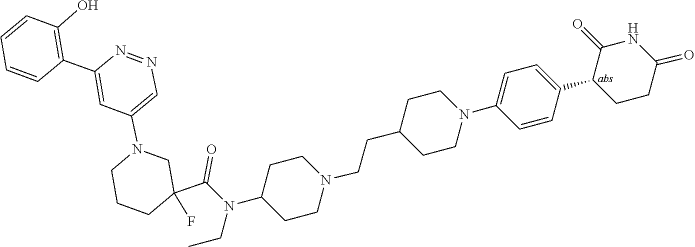 [SMILES: CCN(C(=O)C1(F)CCCN(c2cnnc(-c3ccccc3O)c2)C1)C1CCN(CCC2CCN(c3ccc([C@H]4CCC(=O)NC4=O)cc3)CC2)CC1]

### Step-1: tert-butyl 4-(N-ethyl-3-fluoro-1-(6-(2-(methoxymethoxy)phenyl)pyridazin-4-yl)piperidine-3-carboxamido)piperidine-1-carboxylate

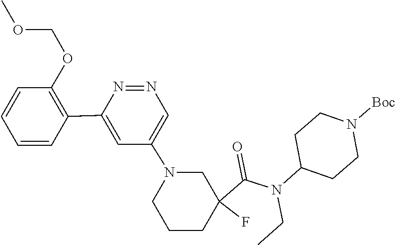 [SMILES: CCN(C(=O)C1(F)CCCN(c2cnnc(-c3ccccc3OCOC)c2)C1)C1CCN(C(=O)OC(C)(C)C)CC1]

To an ice cold stirred solution of 3-fluoro-1-(6-(2-(methoxymethoxy)phenyl)pyridazin-4-yl)piperidine-3-carboxylic acid lithium salt (300 mg, 0.83 mmol) and tert-butyl 4-(ethylamino)piperidine-1-carboxylate (152 mg, 0.66 mmol) in DMF (5 mL) was added DIPEA (0.5 ml, 2.49 mmol) followed by HATU (347 mg, 0.91 mmol) under inert atmosphere and stirred at RT for 16 h. After completion, the reaction mixture was concentrated, quenched with water (20 mL) and extracted with EtOAc (2×20 mL). The organic layer was washed thoroughly with chilled brine solution (2×40 mL). The combined organic layer was dried over anhydrous sodium sulphate, filtered and concentrated under reduced pressure to get crude. Crude product was purified by flash column chromatography (silica gel, 12 g SNAP) using eluents 5-8% MeOH in DCM to get tert-butyl 4-(N-ethyl-3-fluoro-1-(6-(2-(methoxymethoxy)phenyl)pyridazin-4-yl)piperidine-3-carboxamido)piperidine-1-carboxylate as yellow sticky solid (235 mg, 50%). LCMS: m/z 572.15 [M+H]⁺; Purity: 91%.

### Step-2: N-ethyl-3-fluoro-1-(6-(2-hydroxyphenyl)pyridazin-4-yl)-N-(piperidin-4-yl)piperidine-3-carboxamide

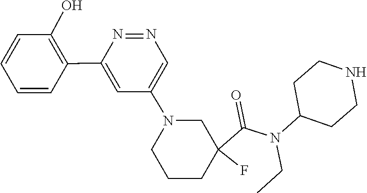 [SMILES: CCN(C(=O)C1(F)CCCN(c2cnnc(-c3ccccc3O)c2)C1)C1CCNCC1]

To an ice-cold stirred solution of tert-butyl 4-(N-ethyl-3-fluoro-1-(6-(2-(methoxymethoxy)phenyl)pyridazin-4-yl)piperidine-3-carboxamido)piperidine-1-carboxylate (210 mg, 0.38 mmol) in DCM (5 mL) was added 4 N HCl in 1,4 Dioxane (2.5 mL) and the reaction mixture was stirred at RT for 5 h. After completion of reaction (monitored by TLC and LCMS), the reaction mixture was concentrated under reduced pressure and triturated with diethyl ether (10 mL) to get N-ethyl-3-fluoro-1-(6-(2-hydroxyphenyl)pyridazin-4-yl)-N-(piperidin-4-yl)piperidine-3-carboxamide as white solid (175 mg, crude). LCMS: m/z 428.05 M+H]⁺; Purity: 93%.

### Step-3: Synthesis of the Title Compound

To an ice cold stirred solution N-ethyl-3-fluoro-1-(6-(2-hydroxyphenyl)pyridazin-4-yl)-N-(piperidin-4-yl)piperidine-3-carboxamide 4 (155 mg, 0.33 mmol) and (R)-2-(1-(4-(2,6-dioxopiperidin-3-yl)phenyl)piperidin-4-yl)acetaldehyde 5 (83 mg, 0.26 mmol) in CH₃CN:MeOH (3:1, 8 mL) was added DIPEA (0.28 mL, 1.65 mmol) and reaction mixture was stirred at RT for 30 min before adding STAB (210 mg, 0.99 mmol) at 0° C. Reaction mixture was stirred at RT for another 3 h. After completion (monitored by TLC and LCMS), the reaction mixture was concentrated, quenched with water (20 mL) and extracted with 10% MeOH:DCM (2×30 mL). The combined organic layer was washed with brine (10 mL), dried over anhydrous sodium sulphate, filtered and concentrated under reduced pressure to get the crude. The crude product was purified by RP-HPLC purification Column: X SELECT FLOURO PHENYL (250×19), 5 μm Mobile phase: 5 mM Ammonium Formate in Water B: —ACN with flow rate 18 ml/min to get N-(1-(2-(1-(4-((R)-2,6-dioxopiperidin-3-yl)phenyl)piperidin-4-yl)ethyl)piperidin-4-yl)-N-ethyl-3-fluoro-1-(6-(2-hydroxyphenyl)pyridazin-4-yl)piperidine-3-carboxamide as an off white solid (80 mg, 30% over two steps). LCMS: m/z 726.62 M+H]⁺; Purity: 97.38%. 1H NMR (400 MHz, DMSO-d6): δ 14.57 (brs, 1H), 10.76, (s, 1H), 8.99-8.97 (m, 1H), 8.14 (d, J=8.0 Hz, 1H), 7.60 (d, J=2.6 Hz, 1H), 7.32 (t, J=7.6 Hz, 1H), 7.02 (d, J=8.4 Hz, 2H), 6.93-6.85 (m, 4H), 4.66-4.55 (m, 1H), 4.29-4.26 (m, 1H), 3.92 (m, 1H), 3.81-3.60 (m, 4H), 3.28-3.20 (m, 2H), 2.94-2.88 (m, 2H), 2.64-2.56 (m, 3H), 2.44-2.43 (m, 1H), 2.25-1.97 (m, 6H), 1.90 (s, 1H), 1.79-1.56 (m, 10H), 1.39-1.34 (m, 3H), 1.23-1.19 (m, 2H) and 1.12-1.05 (m, 3H)

### Example 174

### (R)-3-CYCLOBUTOXY-N-(1-(2-(1-(4-((R)-2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-N-METHYLPYRROLIDINE-3-CARBOXAMIDE

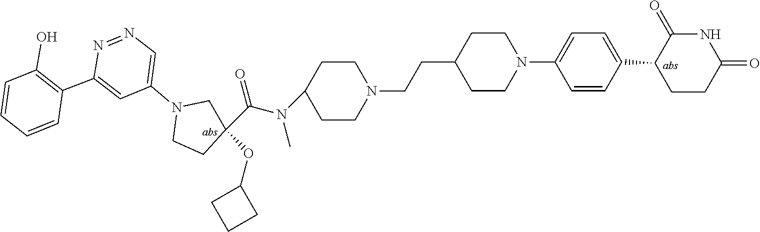 [SMILES: CN(C(=O)[C@@]1(OC2CCC2)CCN(c2cnnc(-c3ccccc3O)c2)C1)C1CCN(CCC2CCN(c3ccc([C@H]4CCC(=O)NC4=O)cc3)CC2)CC1]

### Step 1: SFC Separation of Title Compound

The racemic compound of Example 167A was separated using SFC conditions (Stage 1 column: IBN, Stage 2 column: IH, EtOH+60% CAN+0.1% NH4OH). Peak 2 was collected to yield the title compound (15.8 mg) as a white solid.

### Example 175

### RAC-N-(1-((R)-1-(1-(4-((S)-2,6-DIOXOPIPERIDIN-3-YL)PHENYL)PIPERIDIN-4-YL)PROPAN-2-YL)PIPERIDIN-4-YL)-N-ETHYL-1-(6-(2-HYDROXYPHENYL)PYRIDAZIN-4-YL)-4-METHOXYPIPERIDINE-4-CARBOXAMIDE

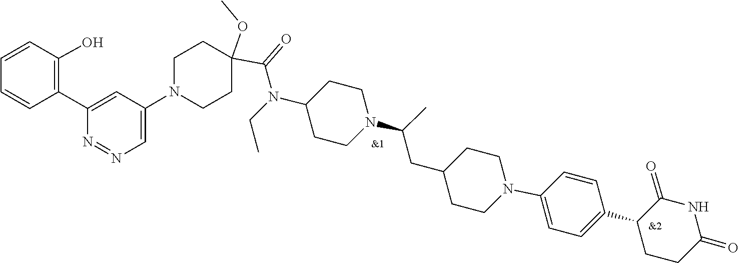 [SMILES: CCN(C(=O)C1(OC)CCN(c2cnnc(-c3ccccc3O)c2)CC1)C1CCN([C@@H](C)CC2CCN(c3ccc([C@H]4CCC(=O)NC4=O)cc3)CC2)CC1]

### Step 1: Synthesis of the Title Compound

N-ethyl-1-[6-(2-hydroxyphenyl)pyridazin-4-yl]-4-methoxy-N-(piperidin-4-yl)piperidine-4-carboxamide hydrochloride (30.00 mg, 0.0630 mmol) was dissolved in DMA (1.00 mL) and (3R)-3-{4-[4-(2-oxopropyl)piperidin-1-yl]phenyl}piperidine-2,6-dione (20.70 mg, 0.0630 mmol) was added in one portion. N,N-diisopropylethylamine (0.06 mL, 40.73 mg, 0.3151 mmol) was then added followed by sodium triacetoxyborohydride (40.07 mg, 0.1891 mmol). The resulting reaction was stirred at RT for 1-2 hrs and monitored via LCMS. Once complete, the reaction was concentrated in vacuo and purified via RP—FC to afford the title compound (4.7 mg, 8.1%) as a formate salt. LCMS: C₄₃H₅₇N₇O₅ requires: 751.4, found: m/z=752.3 [M+H]⁺.

### Example 176

### RAC-N-[1-(6-{4-[(3R)-2,6-DIOXOPIPERIDIN-3-YL]PHENYL}-6-AZASPIRO[3.4]OCTAN-2-YL)PIPERIDIN-4-YL]-N-ETHYL-1-[6-(2-HYDROXYPHENYL)PYRIDAZIN-4-YL]-4-METHOXYPIPERIDINE-4-CARBOXAMIDE

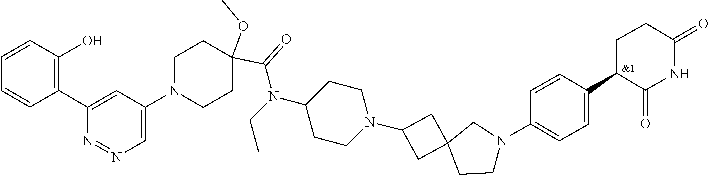 [SMILES: CCN(C(=O)C1(OC)CCN(c2cnnc(-c3ccccc3O)c2)CC1)C1CCN(C2CC3(CCN(c4ccc([C@H]5CCC(=O)NC5=O)cc4)C3)C2)CC1]

Combined N-ethyl-1-[6-(2-hydroxyphenyl)pyridazin-4-yl]-4-methoxy-N-(piperidin-4-yl)piperidine-4-carboxamide hydrochloride (10.10 mg, 0.0212 mmol) and 3-(4-{2-oxo-6-azaspiro[3.4]octan-6-yl}phenyl)piperidine-2,6-dione (7.29 mg, 0.0233 mmol) in DMA (0.30 mL) and then added N,N-diisopropylethylamine (3.71 μL, 0.00 g, 0.0212 mmol) and sodium triacetoxyborohydride (6.75 mg, 0.0318 mmol) and stirred for 3d, then purified directly by RP-HPLC to yield rac-N-[1-(6-{4-[(3R)-2,6-dioxopiperidin-3-yl]phenyl}-6-azaspiro[3.4]octan-2-yl)piperidin-4-yl]-N-ethyl-1-[6-(2-hydroxyphenyl)pyridazin-4-yl]-4-methoxypiperidine-4-carboxamide (0.0084 g, 49.11%). LCMS: C₄₂H₅₃N₇O₅ requires: 735.4, found: m/z=736.3 [M+H]⁺.

### Example 177

### (R)—N-(1-((1-(4-(2,6-DIOXOPIPERIDIN-3-YL)PHENYL)PIPERIDIN-4-YL)METHYL)PIPERIDIN-4-YL)-N-ETHYL-1-(6-(2-HYDROXYPHENYL)PYRIDAZIN-4-YL)-4-METHOXYPIPERIDINE-4-CARBOXAMIDE

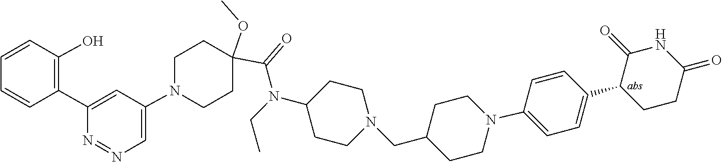 [SMILES: CCN(C(=O)C1(OC)CCN(c2cnnc(-c3ccccc3O)c2)CC1)C1CCN(CC2CCN(c3ccc([C@H]4CCC(=O)NC4=O)cc3)CC2)CC1]

### Step 1: Synthesis of the Title Compound

N-ethyl-1-[6-(2-hydroxyphenyl)pyridazin-4-yl]-4-methoxy-N-(piperidin-4-yl)piperidine-4-carboxamide hydrochloride (30.00 mg, 0.0630 mmol) was dissolved in DMA (1.00 mL) and 1-{4-[(3R)-2,6-dioxopiperidin-3-yl]phenyl}piperidine-4-carbaldehyde (18.93 mg, 0.0630 mmol) was added in one portion. N,N-diisopropylethylamine (40.73 mg, 0.3151 mmol) was then added followed by sodium triacetoxyborohydride (40.07 mg, 0.1891 mmol). The resulting reaction was stirred at RT for 1-2 hrs and monitored via LCMS. Once complete, the reaction was concentrated in vacuo and purified via RP—FC to afford the title compound (25.3 mg, 50.3%) as a formate salt. LCMS: C₄₁H₅₃N₇O₅ requires: 723.4, found: m/z=724.4 [M+H]⁺.

### Example 178

### N—((R)-SEC-BUTYL)-N-(1-(2-(1-(4-((R)-2,6-DIOXOPIPERIDIN-3-YL)PHENYL)PIPERIDIN-4-YL)ETHYL)PIPERIDIN-4-YL)-1-(6-(2-HYDROXYPHENYL)PYRIDAZIN-4-YL)-4-METHOXYPIPERIDINE-4-CARBOXAMIDE

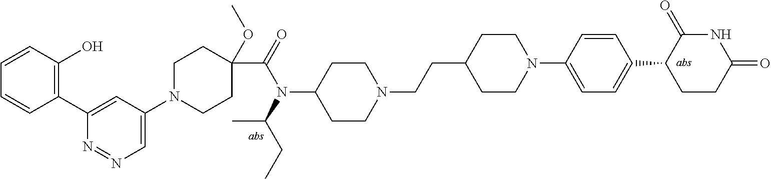 [SMILES: CC[C@@H](C)N(C(=O)C1(OC)CCN(c2cnnc(-c3ccccc3O)c2)CC1)C1CCN(CCC2CCN(c3ccc([C@H]4CCC(=O)NC4=O)cc3)CC2)CC1]

### Step 1: Synthesis of tert-butyl (R)-4-(sec-butylamino)piperidine-1-carboxylate

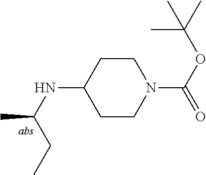 [SMILES: CC[C@@H](C)NC1CCN(C(=O)OC(C)(C)C)CC1]

The title compound was prepared as described for Example 94, Step 1, using (R)-butan-2-amine to afford the desired product as a colorless oil. ¹H NMR (700 MHz, DMSO) δ 3.81 (s, 2H), 2.89-2.69 (m, 2H), 2.66-2.55 (m, 2H), 1.75-1.67 (m, 2H), 1.38 (s, 9H), 1.35 (ddd, J=13.1, 7.5, 5.4 Hz, 1H), 1.20 (dp, J=14.3, 7.3 Hz, 1H), 1.12-0.98 (m, 2H), 0.92 (d, J=6.2 Hz, 3H), 0.82 (t, J=7.4 Hz, 3H). LCMS: C₁₄H₂₈N₂O₂ requires 256.2, found: m/z=257.2 [M+H]⁺.

### Step 2: Synthesis of (R)—N-(sec-butyl)-1-(6-(2-hydroxyphenyl)pyridazin-4-yl)-4-methoxy-N-(piperidin-4-yl)piperidine-4-carboxamide

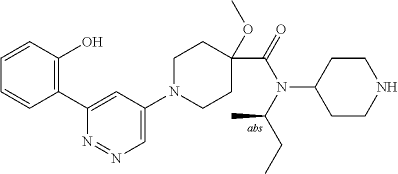 [SMILES: CC[C@@H](C)N(C(=O)C1(OC)CCN(c2cnnc(-c3ccccc3O)c2)CC1)C1CCNCC1]

The title compound was prepared as described for Example 89, Step 1, using tert-butyl (R)-4-(sec-butylamino)piperidine-1-carboxylate to afford the desired product as a light brown solid. LCMS: C₂₆H₃₇N₅O₃ requires 467.3, found: m/z=468.4 [M+H]⁺.

### Step 3: Synthesis of the Title Compound

The title compound was prepared as described for Example 1, Step 2, using (R)—N-(sec-butyl)-1-(6-(2-hydroxyphenyl)pyridazin-4-yl)-4-methoxy-N-(piperidin-4-yl)piperidine-4-carboxamide and (R)-2-(1-(4-(2,6-dioxopiperidin-3-yl)phenyl)piperidin-4-yl)acetaldehyde to afford the title compound (5 mg, 28%). LCMS: C₄₄H₅₉N₇O₅ requires 765.5, found: m/z=766.3 [M+H]⁺.

### Example 179

### RAC-(R)—N-(1-(2-(1-(4-(2,6-DIOXOPIPERIDIN-3-YL)PHENYL)-4-FLUOROPIPERIDIN-4-YL)ETHYL)PIPERIDIN-4-YL)-N-ETHYL-1-(6-(2-HYDROXYPHENYL)PYRIDAZIN-4-YL)-4-METHOXYPIPERIDINE-4-CARBOXAMIDE

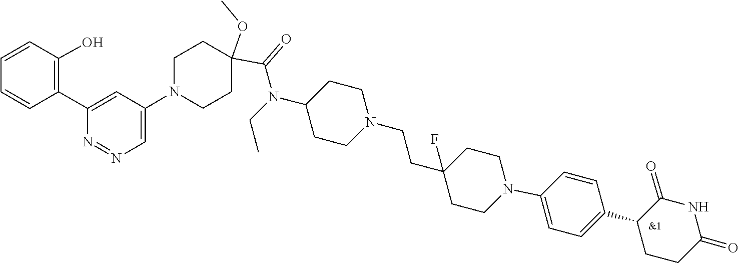 [SMILES: CCN(C(=O)C1(OC)CCN(c2cnnc(-c3ccccc3O)c2)CC1)C1CCN(CCC2(F)CCN(c3ccc([C@H]4CCC(=O)NC4=O)cc3)CC2)CC1]

### Step 1: Synthesis of the Title Compound

N-ethyl-1-[6-(2-hydroxyphenyl)pyridazin-4-yl]-4-methoxy-N-(piperidin-4-yl)piperidine-4-carboxamide hydrochloride (30.00 mg, 0.0630 mmol) was dissolved in DMA (1.00 mL) and 2-(1-{4-[(3R)-2,6-dioxopiperidin-3-yl]phenyl}-4-fluoropiperidin-4-yl)acetaldehyde (20.70 mg, 0.0630 mmol) was added in one portion followed by sodium triacetoxyborohydride (40.07 mg, 0.1891 mmol). The resulting reaction was stirred at RT for 1-2 hrs and monitored via LCMS. Once complete, the reaction was concentrated in vacuo and purified via RP—FC to afford the title compound (22.8 mg, 43.1%) as a formate salt. LCMS: C₄₂H₅₄FN₇O₅ requires: 755.4, found: m/z=756.3 [M+H]⁺.

### Example 180

### (R)-3-(4-((RS)-3-(8-(1-(6-(2-HYDROXYPHENYL)PYRIDAZIN-4-YL)-4-METHOXYPIPERIDINE-4-CARBONYL)-2,8-DIAZASPIRO[4.5]DECAN-2-YL)-1-OXA-8-AZASPIRO[4.5]DECAN-8-YL)PHENYL)PIPERIDINE-2,6-DIONE

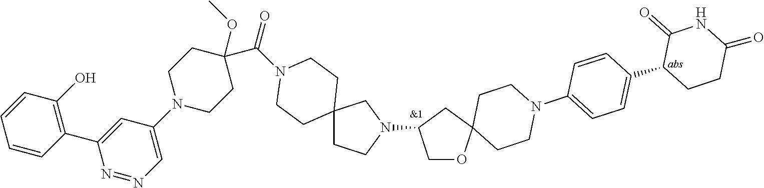 [SMILES: COC1(C(=O)N2CCC3(CC2)CCN([C@H]2COC4(CCN(c5ccc([C@H]6CCC(=O)NC6=O)cc5)CC4)C2)C3)CCN(c2cnnc(-c3ccccc3O)c2)CC1]

### Step 1: Synthesis of (1-(6-(2-hydroxyphenyl)pyridazin-4-yl)-4-methoxypiperidin-4-yl)(2,8-diazaspiro[4.5]decan-8-yl)methanone

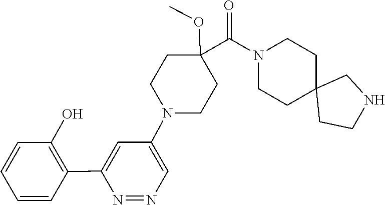 [SMILES: COC1(C(=O)N2CCC3(CCNC3)CC2)CCN(c2cnnc(-c3ccccc3O)c2)CC1]

The title compound was prepared as described for Example 92, Step 1, using tert-butyl 2,8-diazaspiro[4.5]decane-2-carboxylate to afford the desired product as a yellow solid. LCMS: C₂₅H₃₃N₅O₃ requires 451.3, found: m/z=452.4 [M+H]⁺.

### Step 2: Synthesis of the Title Compound

The title compound was prepared as described for Example 1, Step 2, using (1-(6-(2-hydroxyphenyl)pyridazin-4-yl)-4-methoxypiperidin-4-yl)(2,8-diazaspiro[4.5]decan-8-yl)methanone and (R)-3-(4-(3-oxo-1-oxa-8-azaspiro[4.5]decan-8-yl)phenyl)piperidine-2,6-dione to afford the title compound (11.3 mg, 22%). LCMS: C₄₄H₅₅N₇O₆ requires 777.4, found: m/z=778.4 [M+H]⁺.

### Example 181

### N-((1RS,3RS,5SR)-8-(2-(1-(4-((R)-2,6-DIOXOPIPERIDIN-3-YL)PHENYL)PIPERIDIN-4-YL)ETHYL)-8-AZABICYCLO[3.2.1]OCTAN-3-YL)-N-ETHYL-1-(6-(2-HYDROXYPHENYL)PYRIDAZIN-4-YL)-4-METHOXYPIPERIDINE-4-CARBOXAMIDE

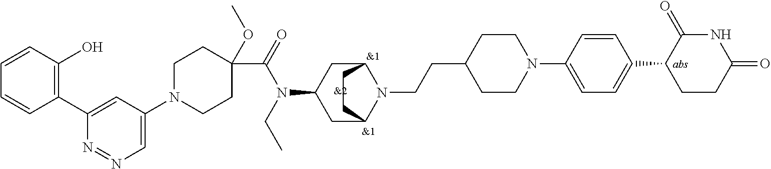 [SMILES: CCN(C(=O)C1(OC)CCN(c2cnnc(-c3ccccc3O)c2)CC1)[C@H]1C[C@H]2CC[C@@H](C1)N2CCC1CCN(c2ccc([C@H]3CCC(=O)NC3=O)cc2)CC1]

### Step 1: Synthesis of N-((1R,3r,5S)-8-azabicyclo[3.2.1]octan-3-yl)-N-ethyl-1-(6-(2-hydroxyphenyl)pyridazin-4-yl)-4-methoxypiperidine-4-carboxamide

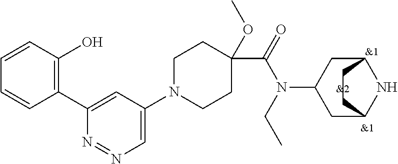 [SMILES: CCN(C(=O)C1(OC)CCN(c2cnnc(-c3ccccc3O)c2)CC1)C1C[C@H]2CC[C@@H](C1)N2]

The title compound was prepared as described for Example 92, Step 1, using tert-butyl (1R,3r,5S)-3-(ethylamino)-8-azabicyclo[3.2.1]octane-8-carboxylate to afford the desired product as a grey solid. LCMS: C₂₆H₃₅N₅O₃ requires 465.3, found: m/z=466.4 [M+H]⁺.

### Step 2: Synthesis of the Title Compound

The title compound was prepared as described for Example 1, Step 2, using N-((1R,3r,5S)-8-azabicyclo[3.2.1]octan-3-yl)-N-ethyl-1-(6-(2-hydroxyphenyl)pyridazin-4-yl)-4-methoxypiperidine-4-carboxamide and (R)-2-(1-(4-(2,6-dioxopiperidin-3-yl)phenyl)piperidin-4-yl)acetaldehyde to afford the title compound (8.3 mg, 16%). LCMS: C₄₄H₅₇N₇O₅ requires 763.4, found: m/z=764.4 [M+H]⁺.

### Example 182

### RAC-N-(1-(((1R,4R)-4-(3-((R)-2,6-DIOXOPIPERIDIN-3-YL)PHENOXY)CYCLOHEXYL)METHYL)PIPERIDIN-4-YL)-N-ETHYL-1-(6-(2-HYDROXYPHENYL)PYRIDAZIN-4-YL)-4-METHOXYPIPERIDINE-4-CARBOXAMIDE

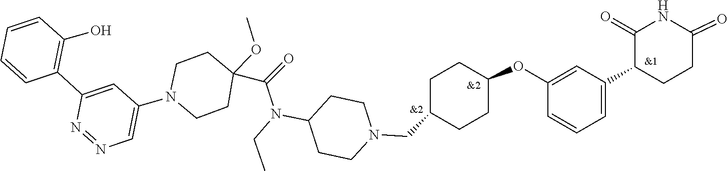 [SMILES: CCN(C(=O)C1(OC)CCN(c2cnnc(-c3ccccc3O)c2)CC1)C1CCN(C[C@H]2CC[C@H](Oc3cccc([C@H]4CCC(=O)NC4=O)c3)CC2)CC1]

### Step 2: Synthesis of the Title Compound

The title compound was prepared as described for Example 1, Step 2, using N-ethyl-1-(6-(2-hydroxyphenyl)pyridazin-4-yl)-4-methoxy-N-(piperidin-4-yl)piperidine-4-carboxamide and rac-(1R,4r)-4-(3-((R)-2,6-dioxopiperidin-3-yl)phenoxy)cyclohexane-1-carbaldehyde to afford the title compound (9 mg, 24%). LCMS: C₄₂H₅₄N₆O₆ requires 738.4, found: m/z=739.3 [M+H]⁺.

### Example 183

### RAC-N-(1-((R)-2-(1-(4-((R)-2,6-DIOXOPIPERIDIN-3-YL)PHENYL)PIPERIDIN-4-YL)PROPYL)PIPERIDIN-4-YL)-N-ETHYL-1-(6-(2-HYDROXYPHENYL)PYRIDAZIN-4-YL)-4-METHOXYPIPERIDINE-4-CARBOXAMIDE

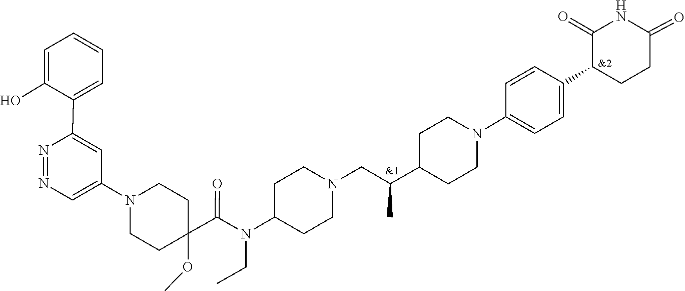 [SMILES: CCN(C(=O)C1(OC)CCN(c2cnnc(-c3ccccc3O)c2)CC1)C1CCN(C[C@H](C)C2CCN(c3ccc([C@H]4CCC(=O)NC4=O)cc3)CC2)CC1]

### Step 1: Synthesis of the Title Compound

N-ethyl-1-[6-(2-hydroxyphenyl)pyridazin-4-yl]-4-methoxy-N-(piperidin-4-yl)piperidine-4-carboxamide hydrochloride (30.00 mg, 0.0630 mmol) was dissolved in DMA (1.00 mL) and 2-(1-{4-[(3R)-2,6-dioxopiperidin-3-yl]phenyl}piperidin-4-yl)propanal (20.70 mg, 0.0630 mmol) was added in one portion. N,N-diisopropylethylamine (0.06 mL, 40.73 mg, 0.3151 mmol) was then added followed by sodium triacetoxyborohydride (40.07 mg, 0.1891 mmol). The resulting reaction was stirred at RT for 1-2 hrs and monitored via LCMS. Once complete, the reaction was concentrated in vacuo and purified via RP—FC to afford the title compound (10.2 mg, 18.8%) as a formate salt. LCMS: C₄₃H₅₇N₇O₅ requires: 751.4, found: m/z=752.3 [M+H]⁺.

### Examples 184-263

The following additional compounds were prepared according to the synthetic schemes described herein.

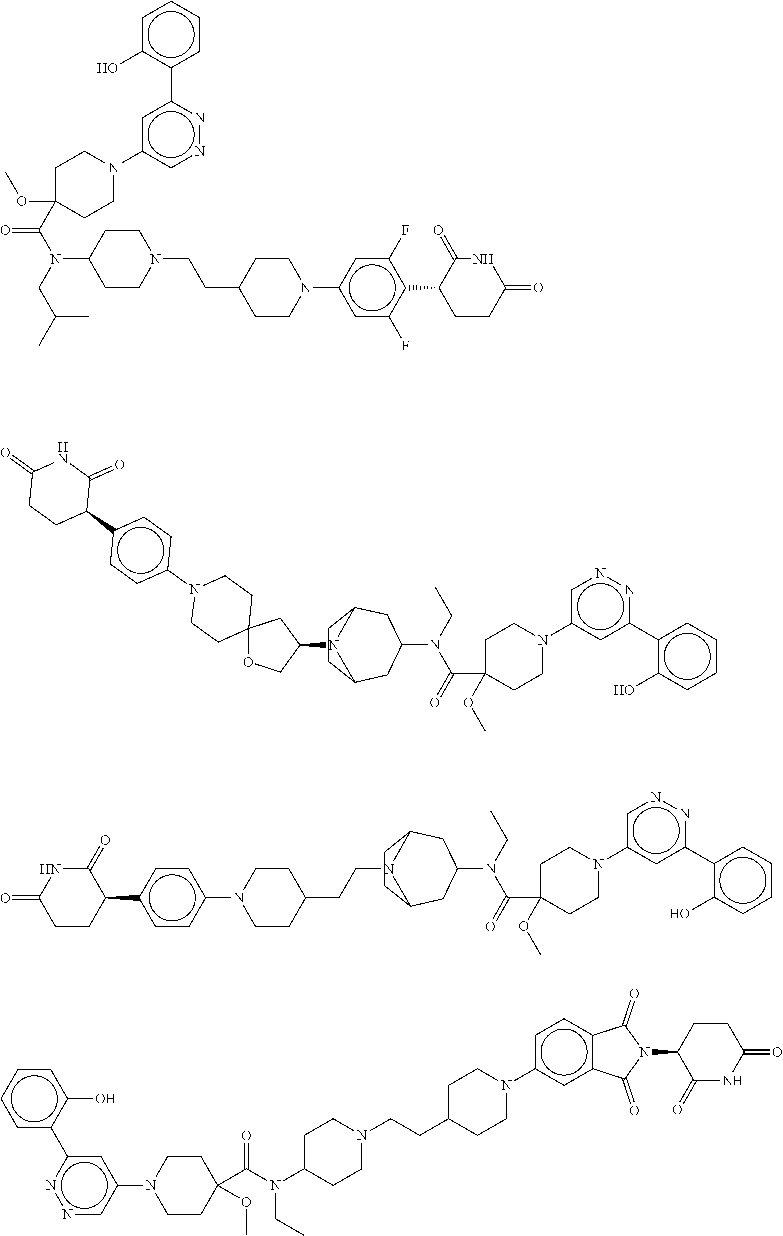

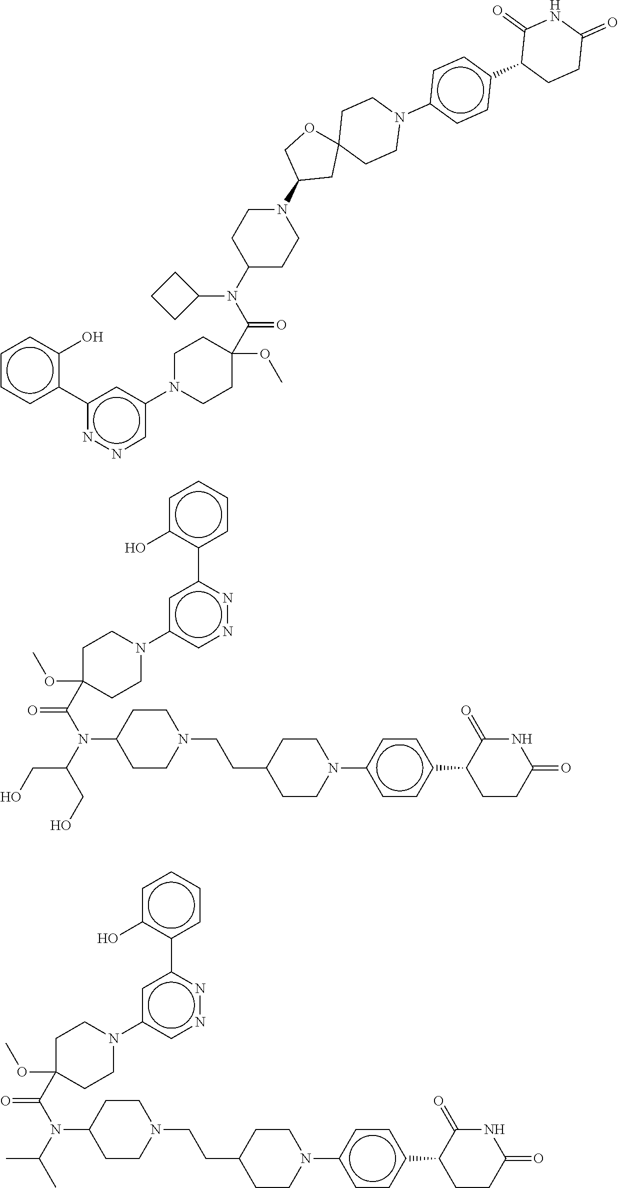

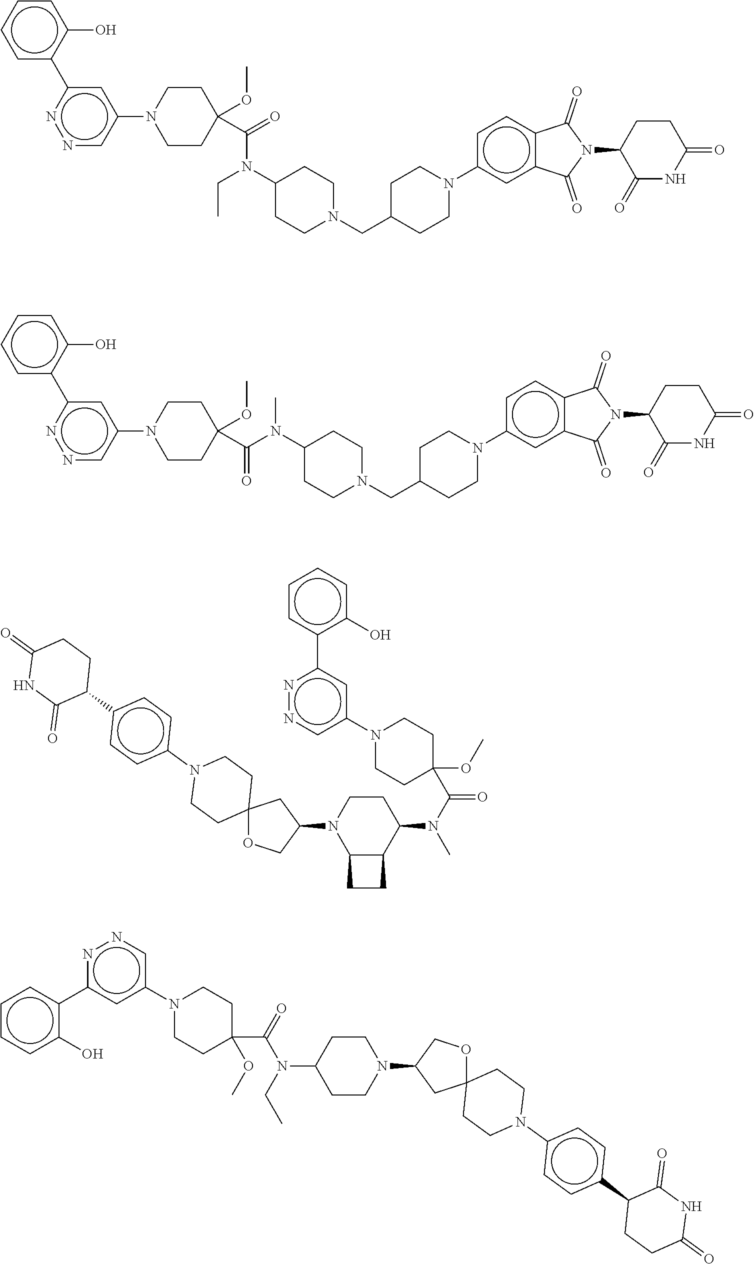

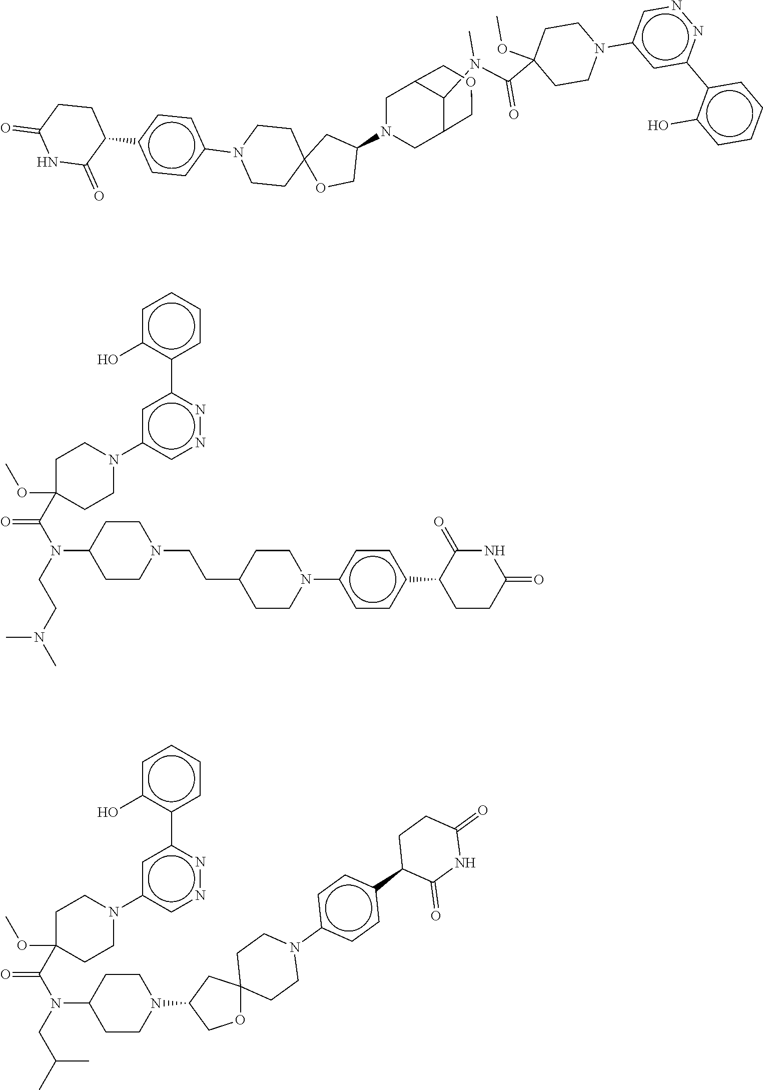

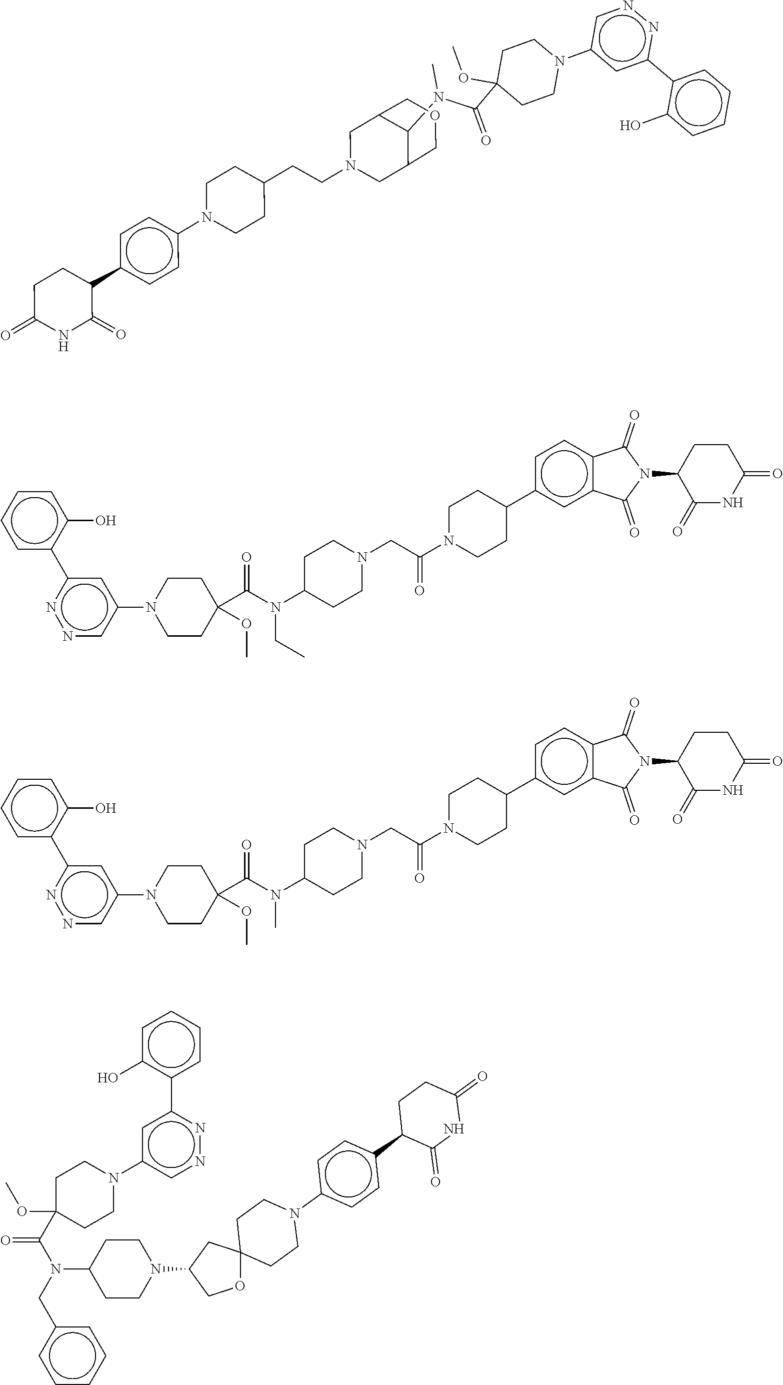

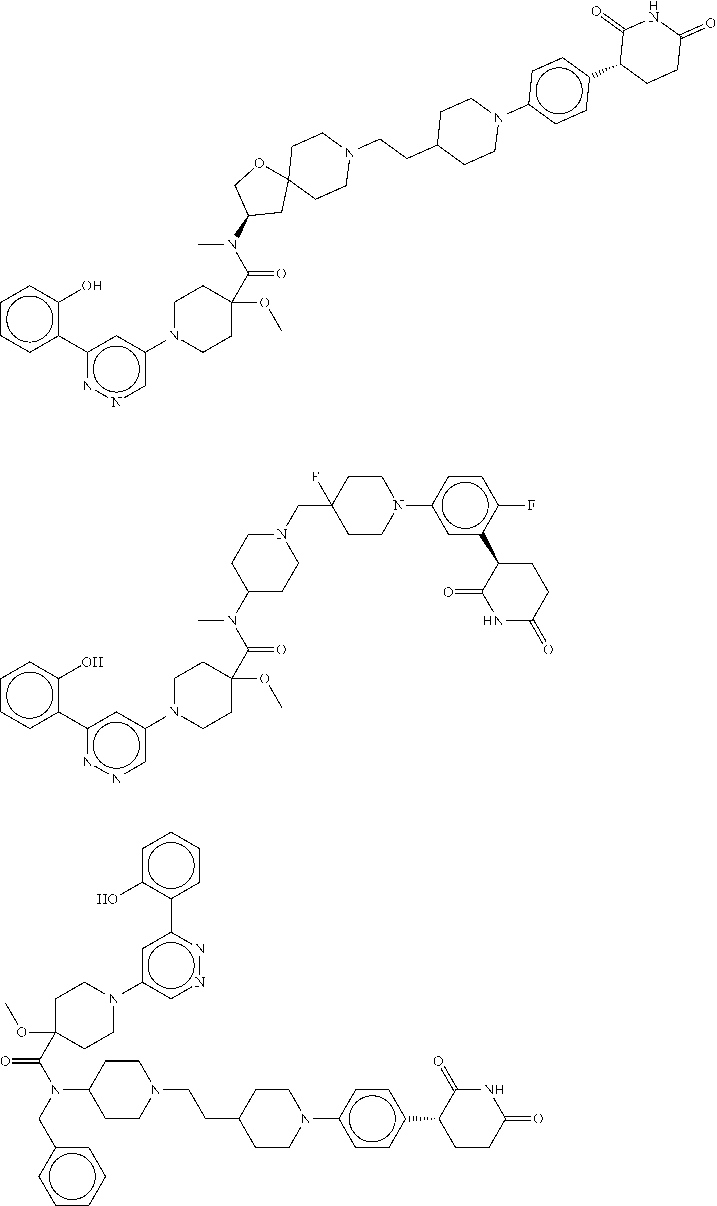

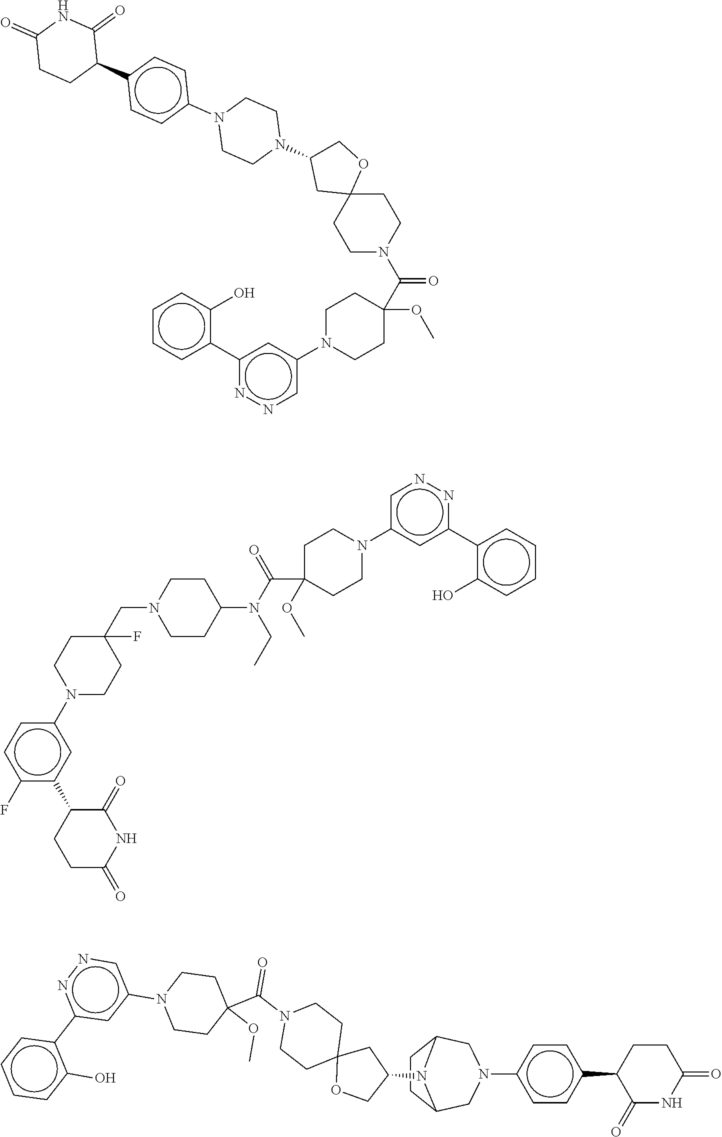

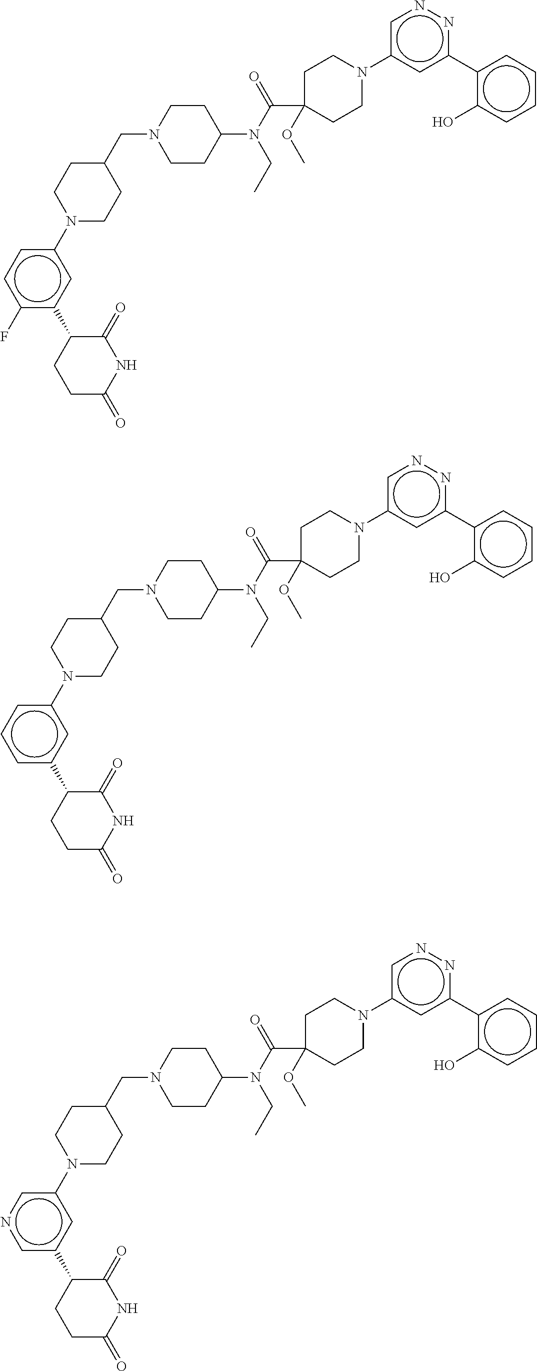

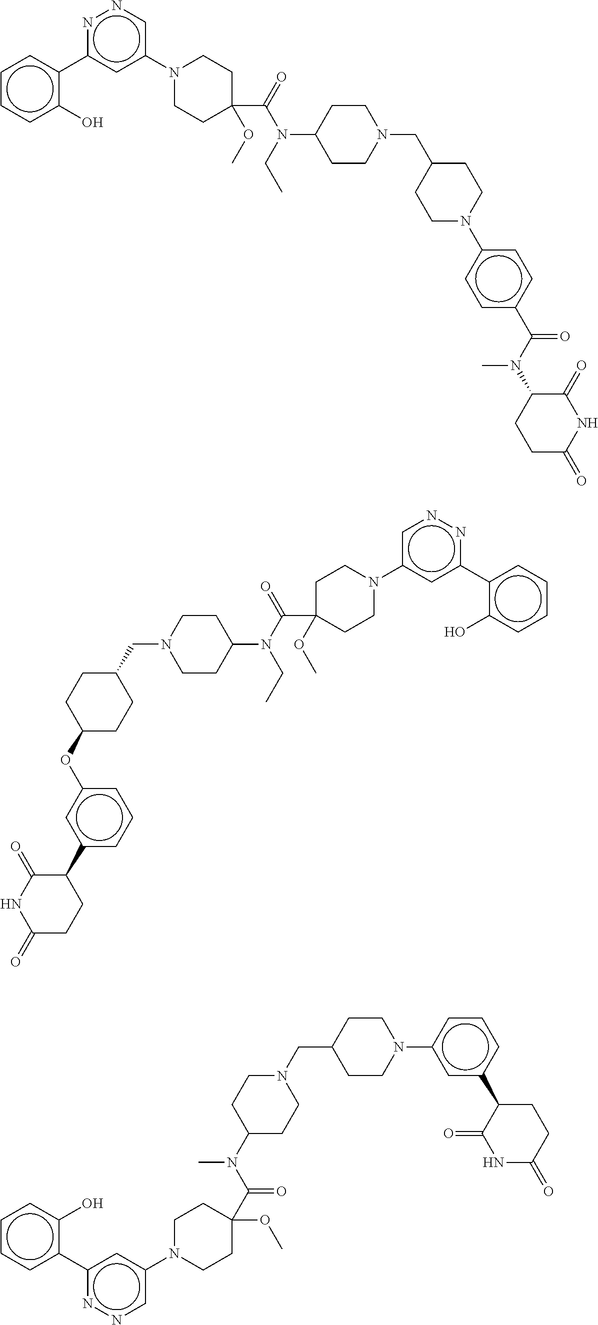

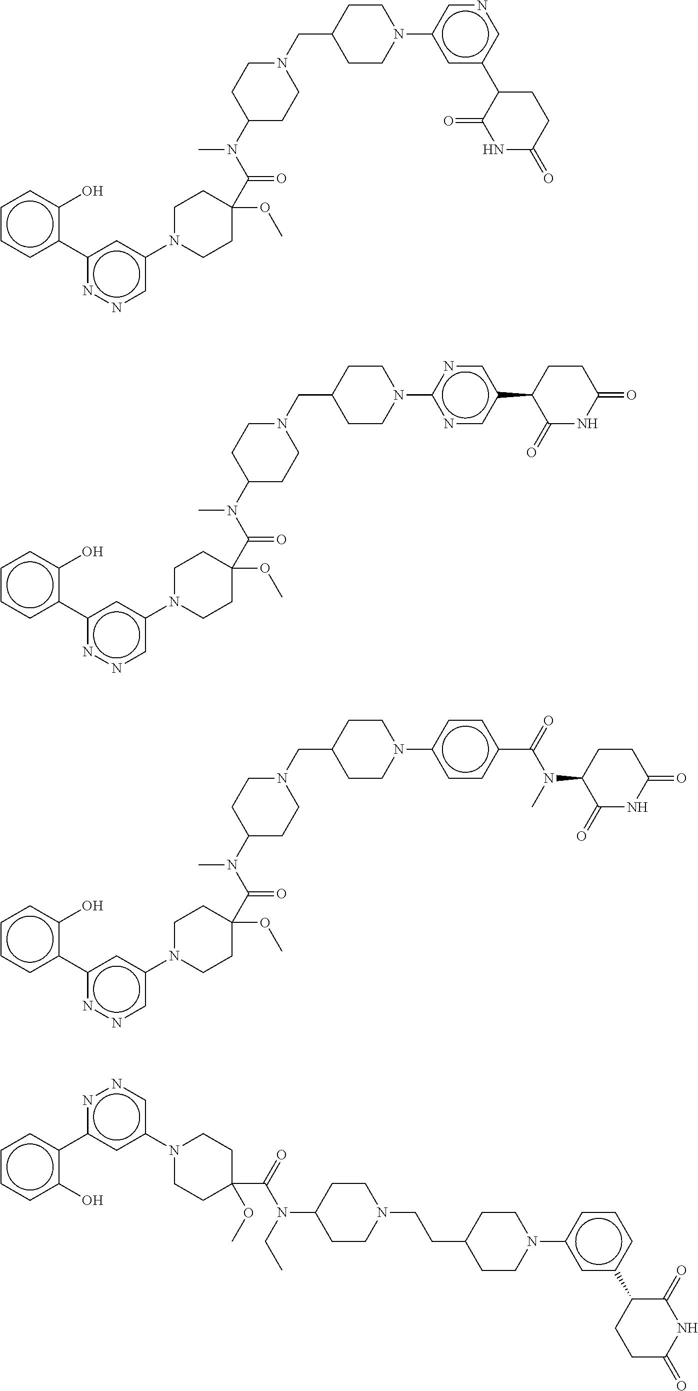

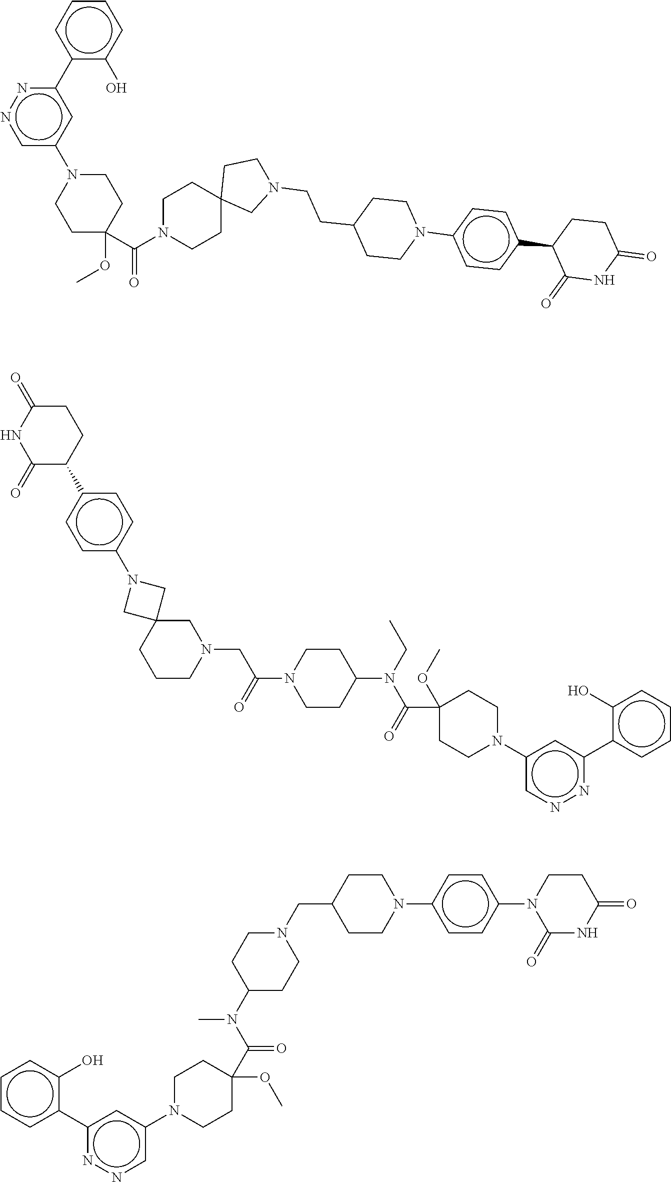

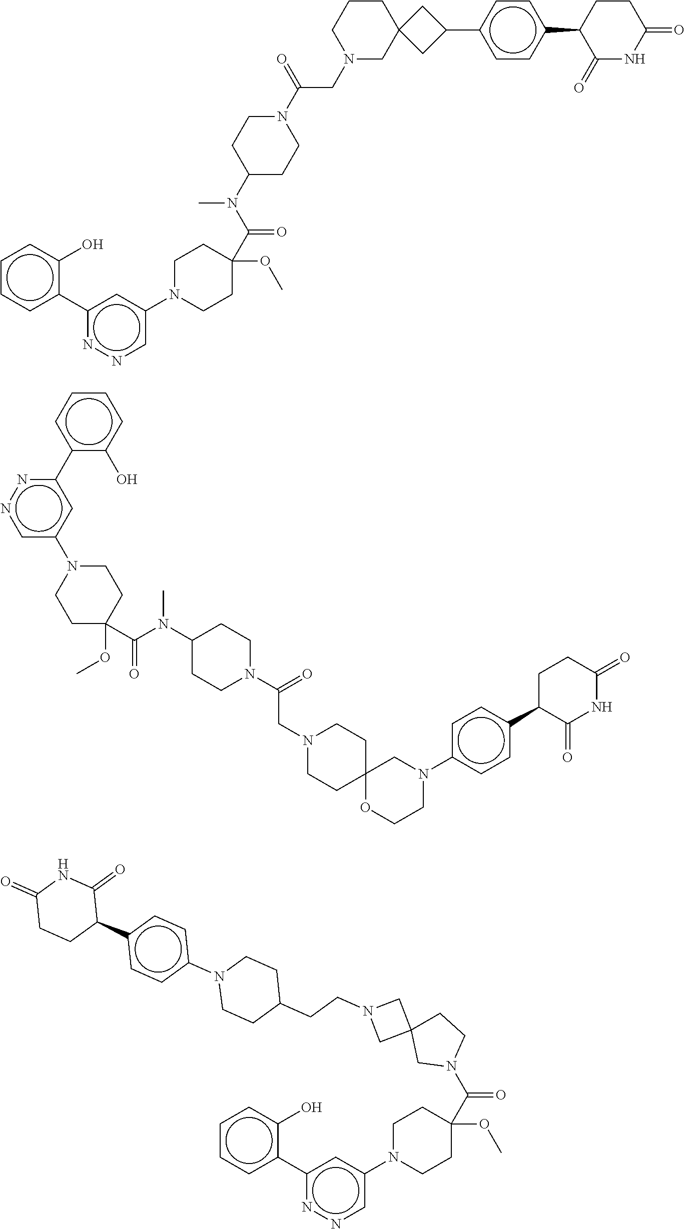

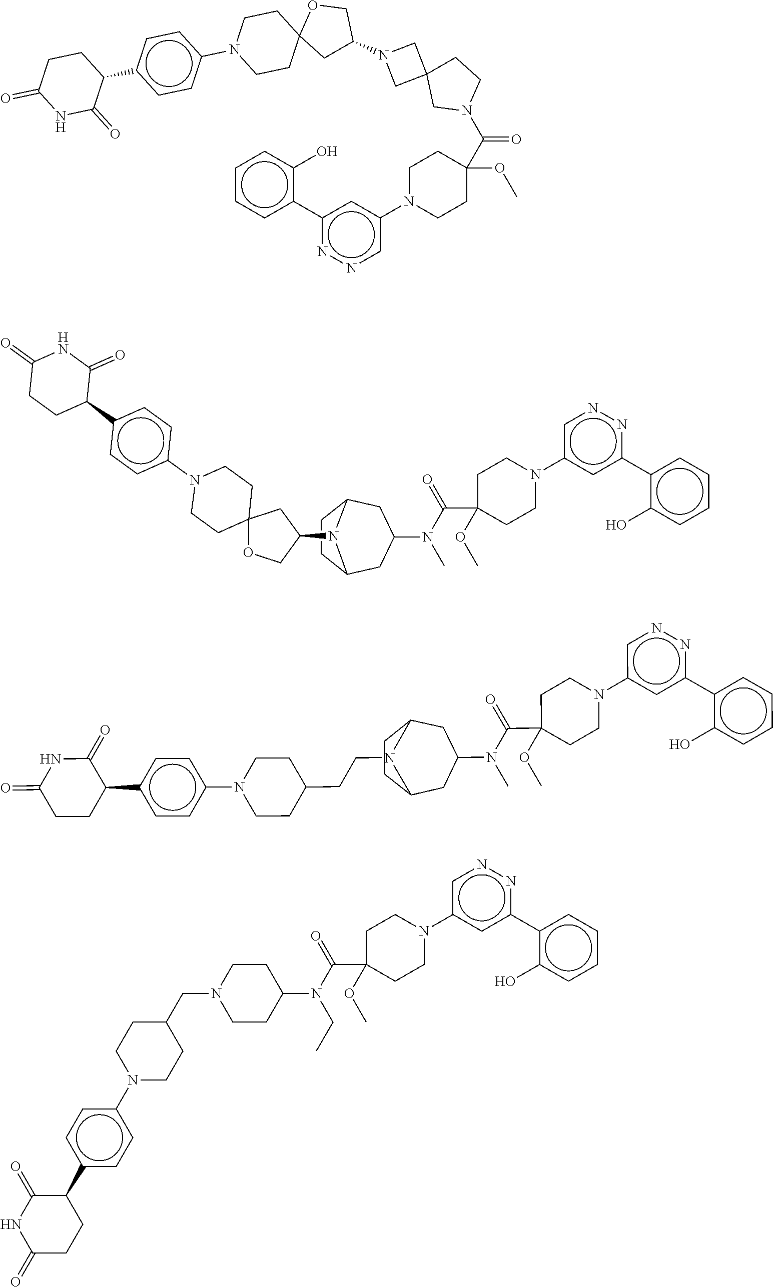

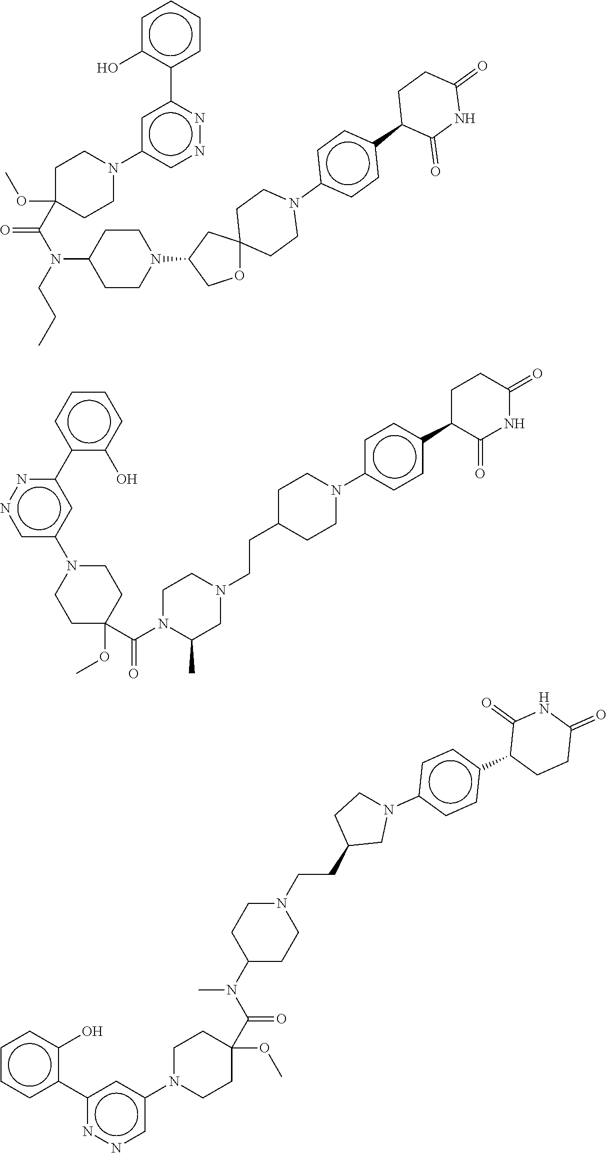

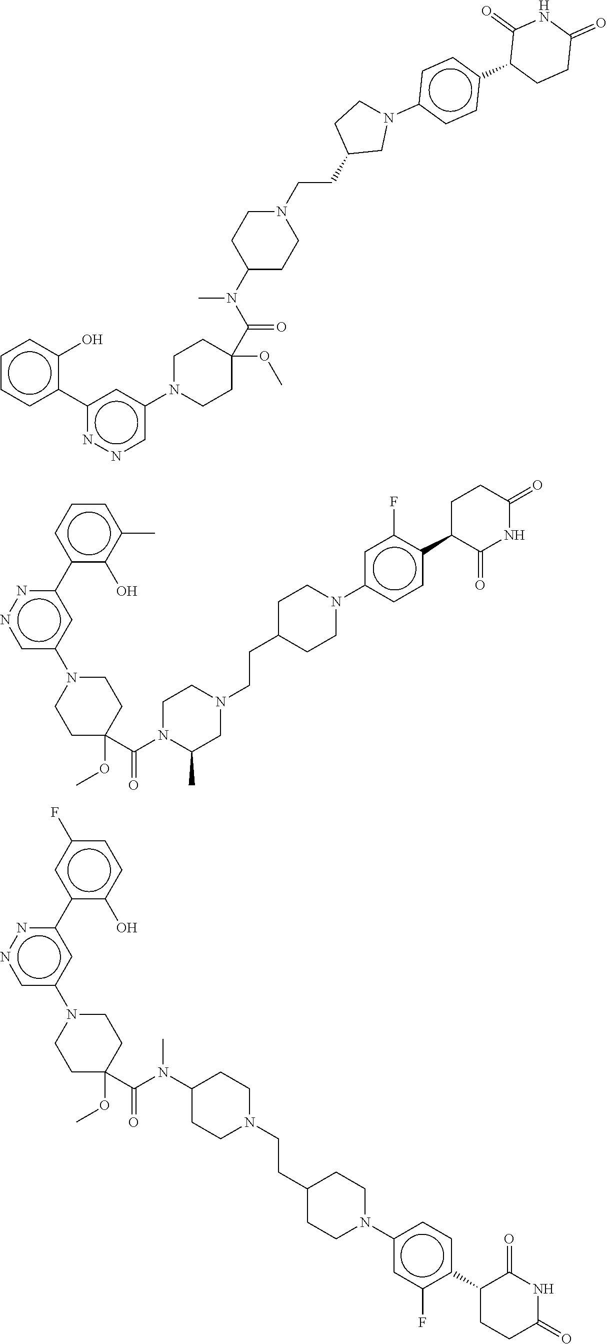

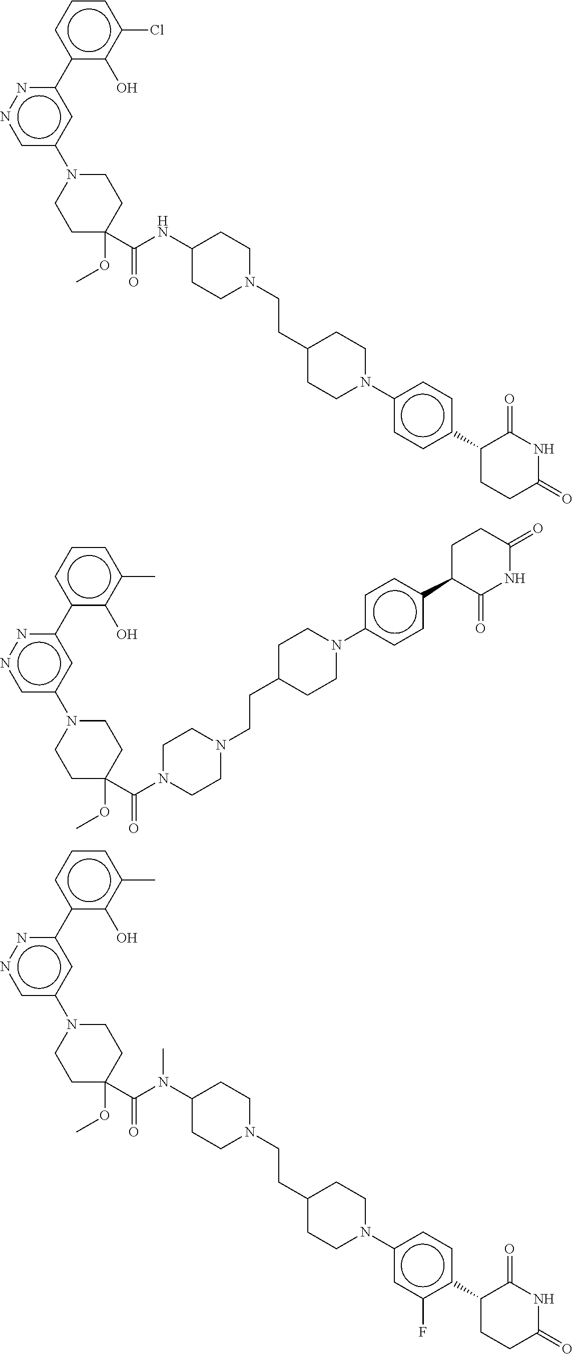

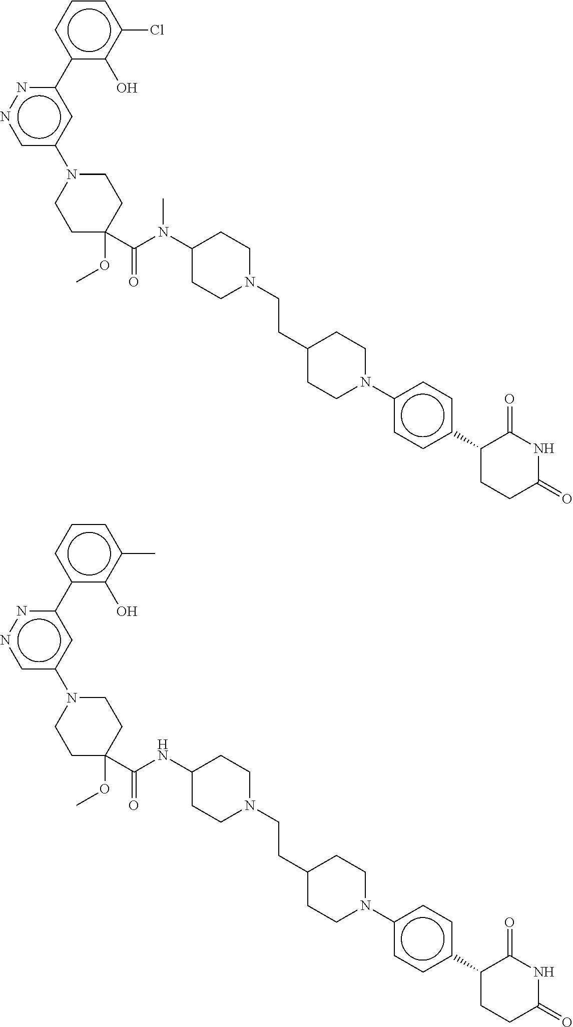

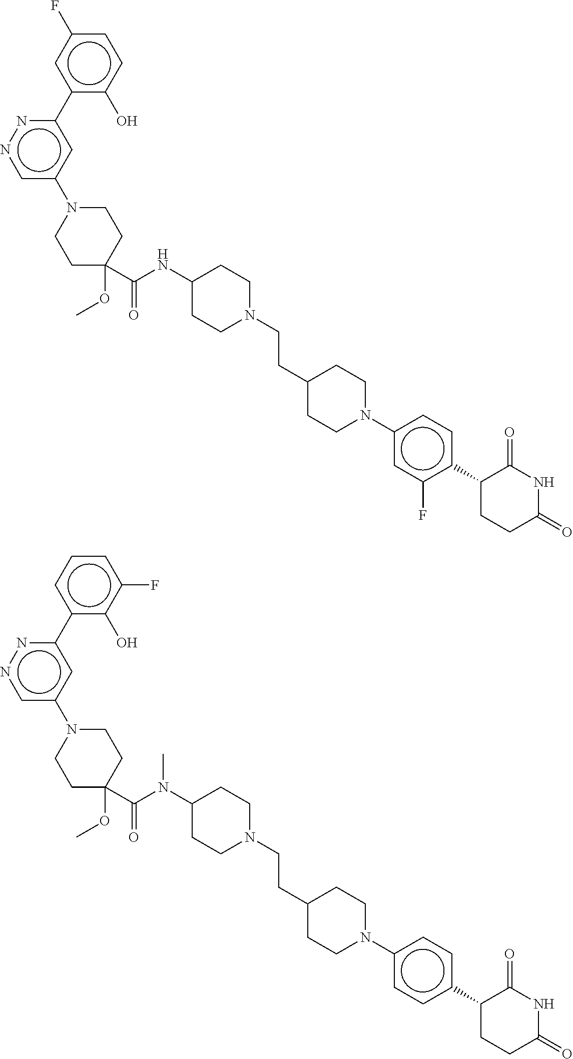

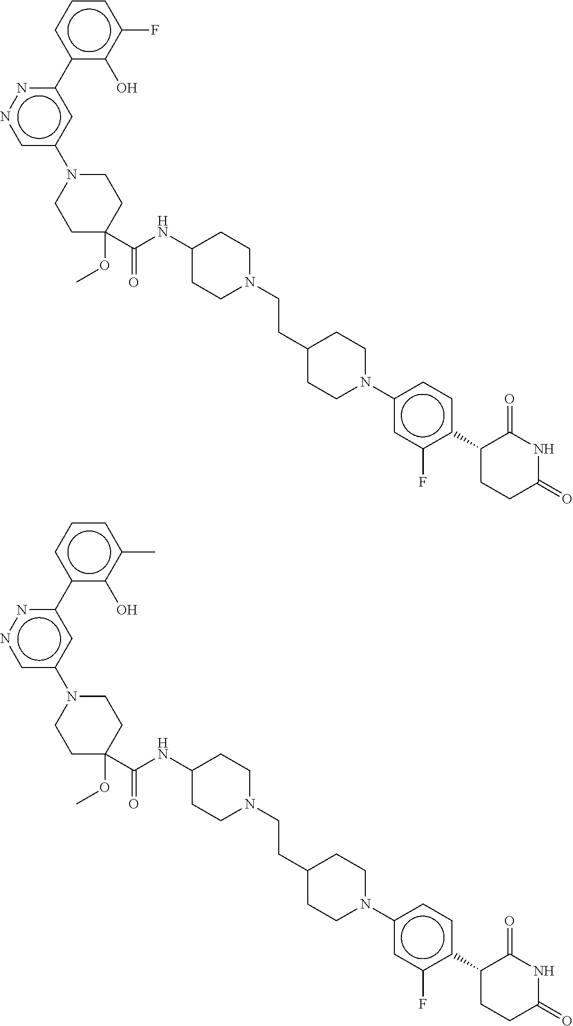

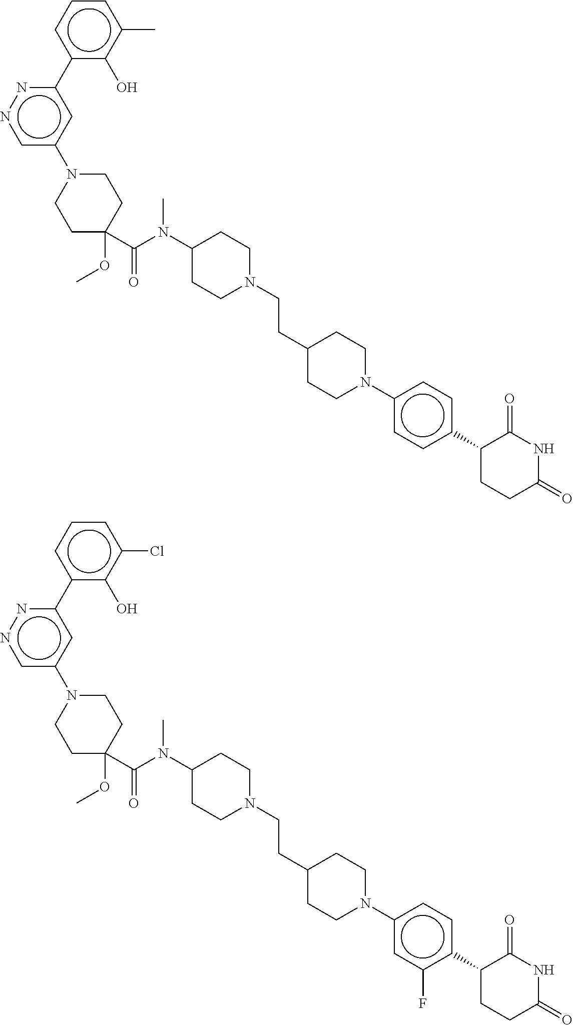

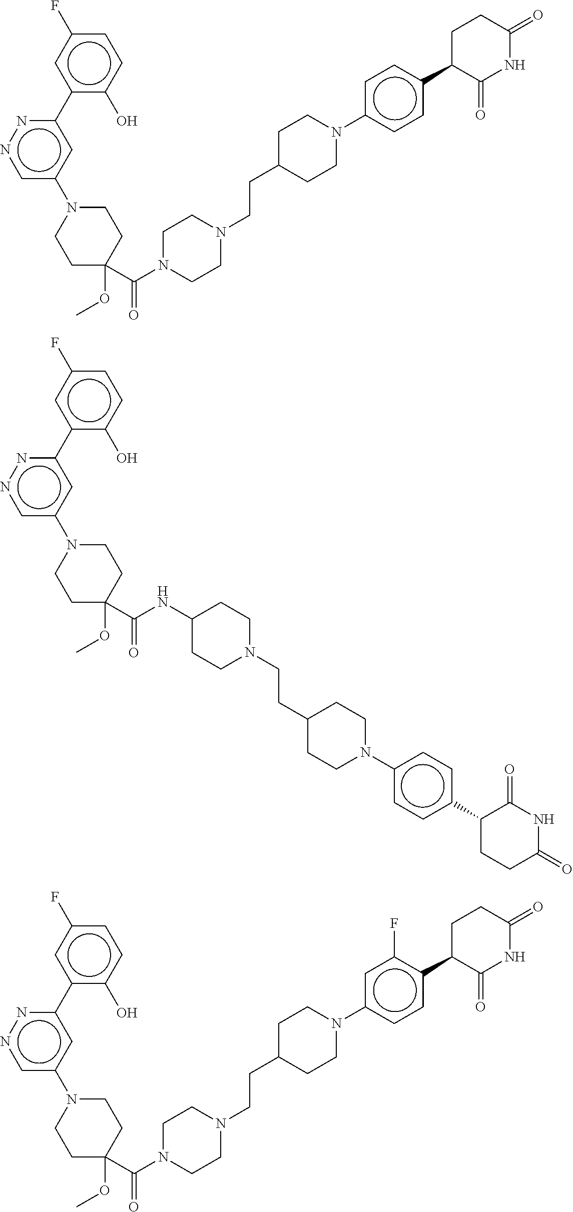

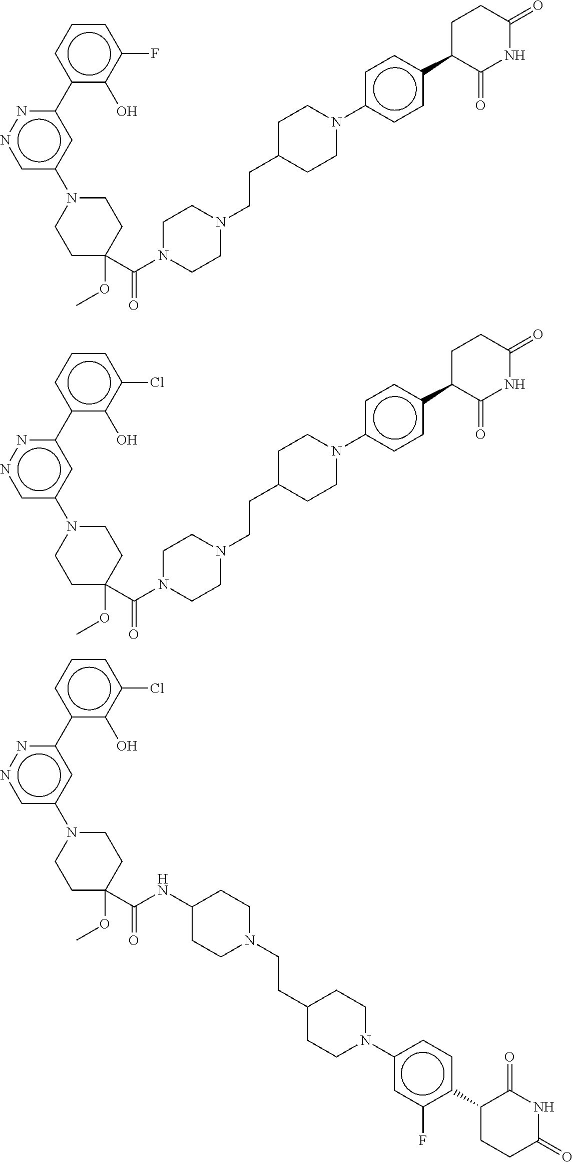

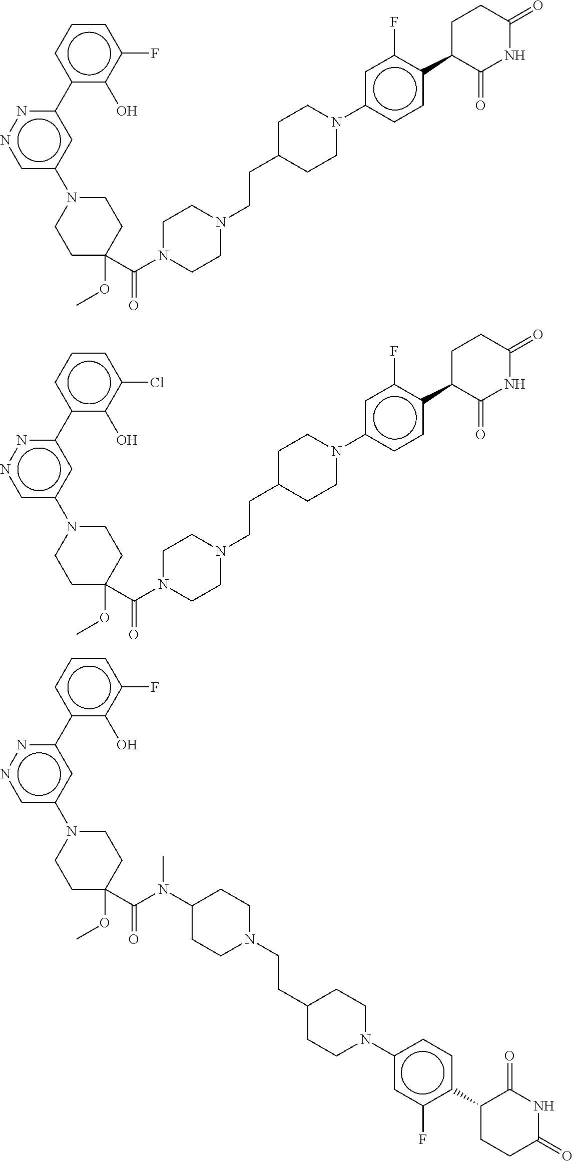

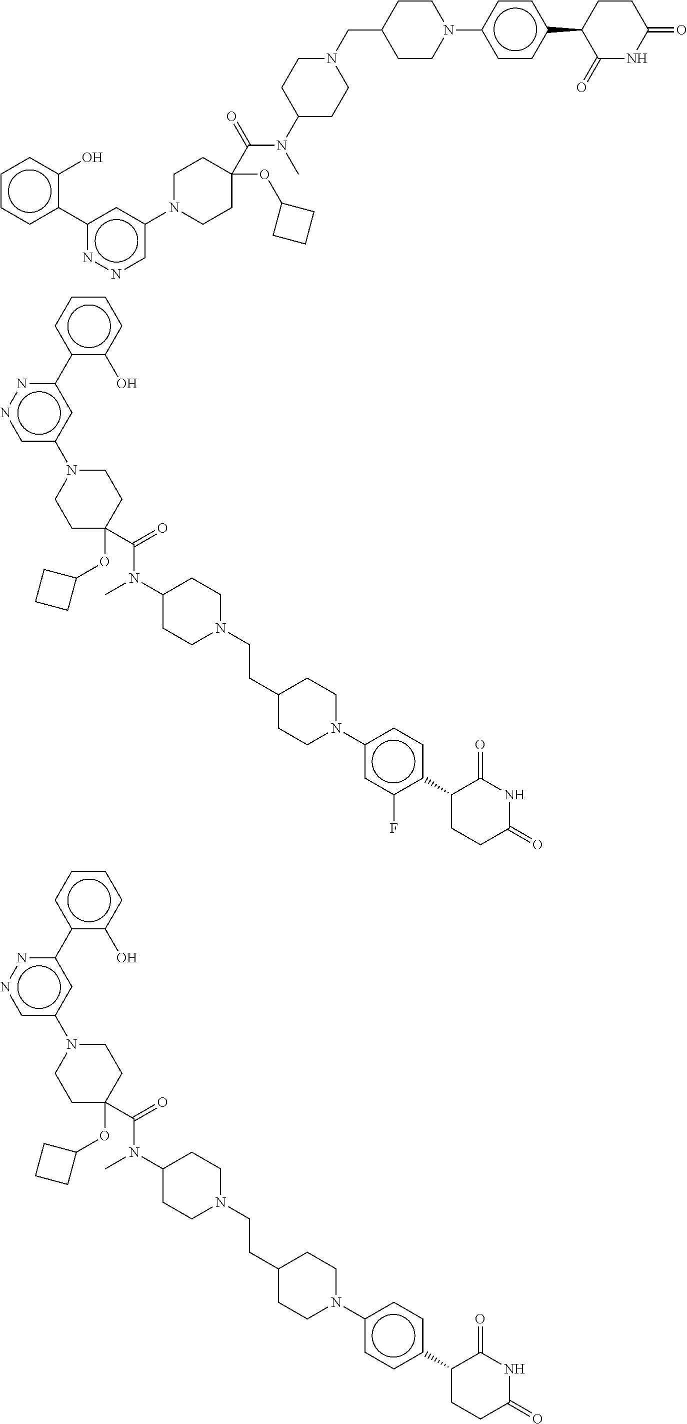

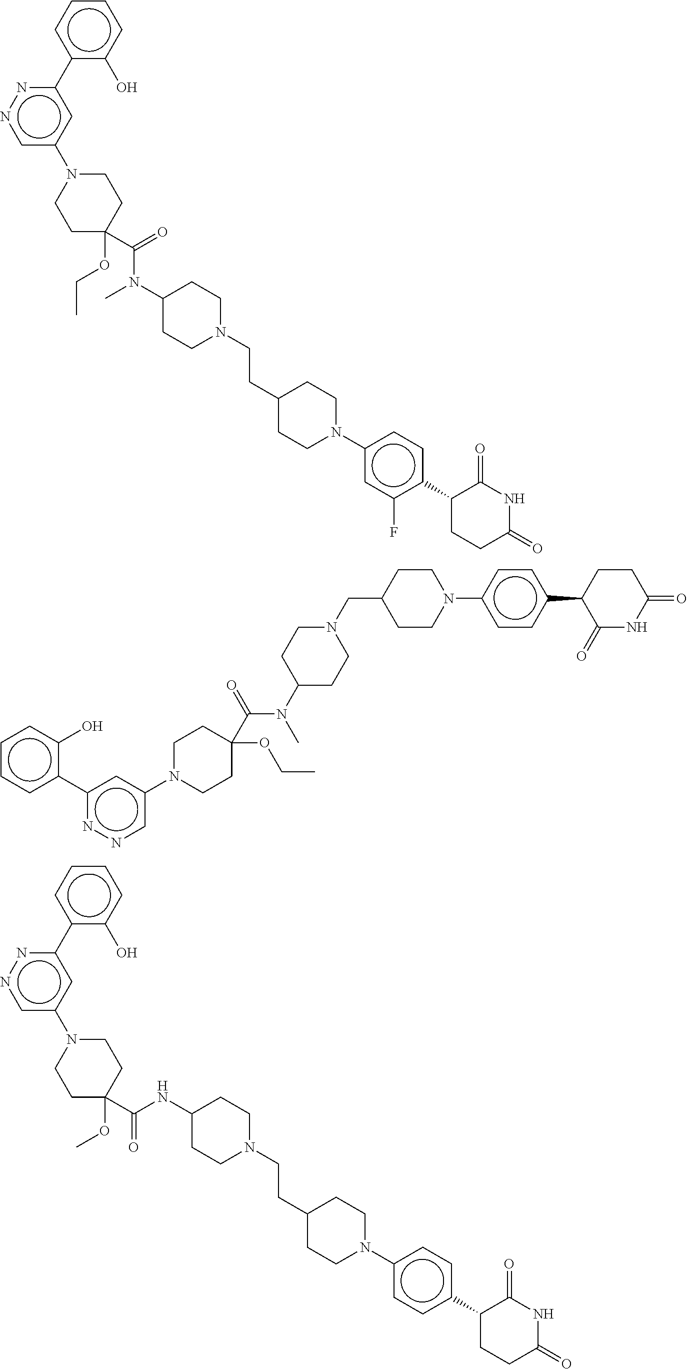

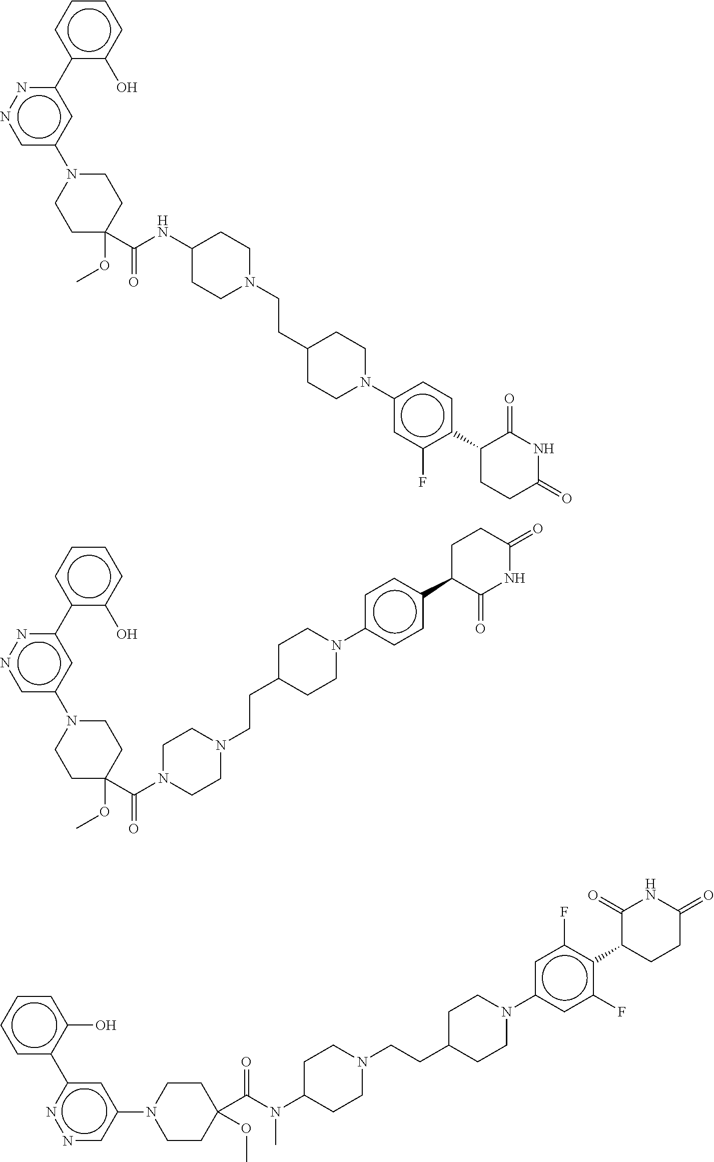

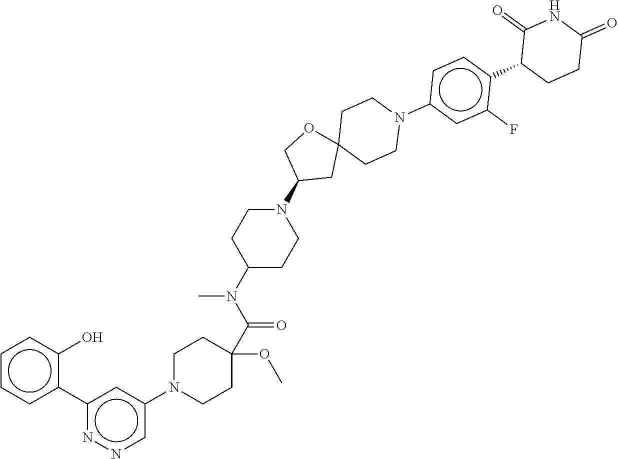

### Biological Example

### SMARCA2 Cellular Degradation Assay

### Western Blot Analysis for SMARCA2 Degradation

Frozen cell pellets were thawed on ice and resuspended in RIPA lysis buffer (Millipore, Cat. No. 20-188) supplemented with 0.2% SDS (Fisher Scientific, Cat. No. 24730020), 200 mM β-glycerophosphate (Alfa Aesar, Cat. No. J62121), 500 mM NaF (Rigaku, Cat. No. 1008046), protease inhibitor (1 tablet per 10 mL, Sigma, Cat. No. 11836170001), 250 U/μL benzonase (Millipore, Cat. No. 71206), 200 mM sodium orthovanadate (FivePhoton, Cat. No. ActVO-4), and 100 mM PMSF (Sigma, Cat. No. 10837091001). 50 μL lysis buffer was used to lyse each pellet containing 5×10E5-1×10E6 live cells extracted from tumors. Lysates were centrifuged for 10 mins at 20,817×g and lysate supernatants were saved for further analysis. Protein levels were determined by the BCA Assay performed according to manufacturer's protocol (EMD Millipore, Cat. No. 71285-3). Samples were evaluated by Western blot analysis on the Jess Platform (ProteinSimple). Lysates were diluted with complete lysis buffer (as described above) and 5 μg of total proteins were loaded onto each capillary. Samples were prepared using the ProteinSimple EZ Standard Pack 1 (Cat. No. PS-ST01EZ-8; containing tubes with powdered (5×) Fluorescent Master Mix, DTT, and biotinylated ladder) and run using the ProteinSimple 25-Capillary Cartridge kit (Cat. No. SM-W004; containing 25-capillary cartridge Wash reagent, and 10× Sample Buffer reagent), the ProteinSimple Anti-Rabbit HRP Detection Module for Jess (Cat. No. DM-001; containing Antibody Diluent, anti-rabbit secondary antibody, Luminol-S, Peroxide, and Streptavidin-HRP), and the ProteinSimple Anti-Mouse NIR Detection Module for Jess (Cat. No. DM-009; containing Antibody Diluent, anti-mouse NIR secondary antibody, and Streptavidin-NIR) as the following describes. The DTT was reconstituted with 40 μL water to make a 400 mM solution, 20 μL of the DTT solution plus 20 μL of 10× Sample Buffer were added to reconstitute the 5× Fluorescent Master Mix, and 20 μL of H₂O was used to reconstitute the biotinylated ladder. For each sample, lysate (4 μL) was combined with 5× Fluorescent Master Mix (1 μL) and heated at 95° C. for 5 minutes. Samples were cooled and stored on ice. Primary antibody solution was made using anti-SMARCA2 antibody (Abcam, Cat. No. 240648, 1:10) and anti-beta-Actin antibody (Cell Signaling Technology, Cat. No. 3700S, 1:25) in antibody diluent. The 25-capillary cartridges were prepared per standard ProteinSimple protocol with each sample in a column. For the biotinylated ladder in column 1, the following were added: 5 μL of ladder sample in Row A, 10 μL of antibody diluent in Rows B and C, 10 μL of Streptavidin-NIR in Row D, and 15 μL of a 50:50 Luminol-s/Peroxide solution in Row E. For each test sample (columns 2-25), the following were added per column: 5 μL of sample in Row A, 10 μL of antibody diluent in Row B, 10 μL of primary antibody solution in Row C, 10 μL of secondary antibody in Row D, and 15 μL of a 50:50 Luminol-S/Peroxide solution in Row E. 500 μL wash buffer was added to the wash buffer wells in the cartridge and cartridge was spun at 1000×g for 5 minutes prior to loading into the Jess. Samples were run using the Chemiluminescence and Fluorescence immunoassays in the ProteinSimple Compass for SimpleWestern program (version 5.0.0). After running, SMARCA2, actin, and background curves were identified in the Compass software; note that in the Compass software, the electropherograms are also visualized as bands for comparison with standard Western blot outputs. The electropherogram area reading for each sample curve was normalized to that of the corresponding actin curve per column following background (capillary background) subtraction using the following equation:

$$Sample Normalized Value = [ \frac{SMARCA 2 Area Va1ue}{β - Actin Area Va1ue} ]$$

Following normalization, the percentage of SMARCA2 remaining per sample relative to the vehicle control was calculated as follows:

$$\% remaining = [ \frac{Example \# / Treated Samp1e Normalized Va1ue}{Mean ( Vehicle Sample Normalized Value} ] \times 100$$

Table 2 demonstrates the biological activities of certain representative compounds of Formula (I).

TABLE 2
Biological Data

| | DC50 (nM) | | Dmax (%) | |
|---|---|---|---|---|
| | SMARCA2 | SMARCA4 | SMARCA2 | SMARCA4 |
| Example | Calu-6 | Calu-6 | Calu-6 | Calu-6 |
| # | C-3E9) | C-5A6) | C-3E9) [%] | C-5A6) [%] |
| 1 | 6.4 | 10000 | 87.06 | 35.88 |
| 2 | 1.97 | 10000 | 90.45 | 44.52 |
| 3 | 15.6 | 10000 | 71.44 | 15.9 |
| 4 | 2.18 | 10000 | 93.42 | 46.55 |
| 5 | 18.03 | 1000 | 69.06 | 25.04 |
| 6 | 13.22 | 10000 | 72.5 | 48.87 |
| 7 | 48.06 | 10000 | 67.17 | 24.25 |
| 8 | 2.95 | 10000 | 86.36 | 37.03 |
| 9 | 7.17 | 10000 | 79.63 | 23.38 |
| 10 | 18.69 | 10000 | 73.39 | 46.1 |
| 11 | 5.82 | 10000 | 86.92 | 39.4 |
| 12 | 18.62 | 10000 | 74.34 | 49.26 |
| 13 | 203.1 | 10000 | 60.85 | 6.11 |
| 14 | 21.78 | 10000 | 69.81 | 13.11 |
| 15 | 6.64 | 10000 | 84.34 | 29.48 |
| 16 | 12.08 | 10000 | 79.58 | 40.97 |
| 17 | 5.72 | 10000 | 78.64 | 25.98 |
| 18 | 24.57 | 10000 | 66.09 | 51.98 |
| 19 | 121.07 | 10000 | 63.24 | 32.01 |
| 20 | 5.35 | 10000 | 83.19 | 35 |
| 21 | 7.35 | 41.3 | 82.68 | 61.8 |
| 22 | 10000 | 10000 | 39.85 | 17.92 |
| 23 | 51.45 | 10000 | 73.4 | 42.21 |
| 24 | 13.72 | 10000 | 69.92 | 39.77 |
| 25 | 37.75 | 10000 | 67.18 | 40.67 |
| 26 | 3.56 | 10000 | 86.39 | 36.85 |
| 27 | 1.84 | 63.61 | 92.93 | 59.09 |
| 28 | 5.69 | 10000 | 84.2 | 27.29 |
| 29 | 11.99 | 10000 | 70.42 | 13.51 |
| 30 | 2.27 | 10000 | 91.16 | 44.44 |
| 31 | 1.18 | 94.39 | 92.77 | 53.73 |
| 32 | 35.7 | 10000 | 72.42 | 35.4 |
| 33 | 12.98 | 10000 | 75.21 | 18.32 |
| 34 | 2.98 | 10000 | 87.82 | 38.22 |
| 35 | 5.6 | 10000 | 80.97 | 27.3 |
| 36 | 16.56 | 10000 | 72.45 | 24.3 |
| 37 | 3.72 | 27.59 | 86.33 | 64.22 |
| 38 | 10000 | 10000 | 27.2 | 21.1 |
| 39 | 10.64 | 541.1 | 76.12 | 35.7 |
| 40 | 144.15 | 10000 | 55.27 | 22.99 |
| 41 | 180.98 | 10000 | 52.96 | 30.87 |
| 42 | 24.63 | 10000 | 58.75 | 17.94 |
| 43 | 33.18 | 10000 | 71.65 | 29.85 |
| 45 | 44.5 | 10000 | 67.08 | 32.17 |
| 46 | 143.96 | 10000 | 55.03 | 15.45 |
| 47 | 50.69 | 10000 | 56.25 | 37.82 |
| 48 | 8.14 | 10000 | 77.68 | 38.7 |
| 49 | 2.55 | 10000 | 90.47 | 33.56 |
| 50 | 22.42 | 10000 | 61.65 | 17.46 |
| 51 | 10000 | 10000 | 50.38 | 22.14 |
| 52 | 4.42 | 174.83 | 87.76 | 54.51 |
| 53 | 26.93 | 10000 | 65.34 | 21.27 |
| 54 | 10000 | 10000 | 42.00 | 21.00 |
| 55 | 14.93 | 10000 | 72.46 | 23.08 |
| 56 | 10.97 | 10000 | 79.73 | 40.37 |
| 57 | 12 | 10000 | 81 | 26 |
| 58 | 3.4 | 10000 | 89.13 | 31.88 |
| 59 | 5.82 | 10000 | 85.68 | 23.29 |
| 60 | 1.63 | 10000 | 90.35 | 48.5 |
| 61 | 1.57 | 71.11 | 89.53 | 55.44 |
| 62 | 1.51 | 44.8 | 89.82 | 52.2 |
| 64 | 1.81 | 41.7 | 88.55 | 58.87 |
| 63 | 2.19 | 61.81 | 84.72 | 51.38 |
| 65 | 4.24 | 10000 | 83.65 | 37.86 |
| 66 | 7.04 | 10000 | 81.81 | 33.06 |
| 67 | 4.06 | 10000 | 83.67 | 28.66 |
| 68 | 6.05 | 10000 | 80.45 | 27.43 |
| 69 | 5.42 | 10000 | 82.63 | 29.99 |
| 70 | 6.87 | 10000 | 81.39 | 28.3 |
| 71 | 4.65 | 10000 | 82.87 | 33.65 |
| 72 | 4.82 | 10000 | 82.98 | 32.88 |
| 73 | 2.98 | 10000 | 84.92 | 35.89 |
| 74 | 17.37 | 10000 | 69.54 | 13.8 |
| 75 | 5.25 | 10000 | 85.73 | 33.58 |
| 76 | 9.08 | 10000 | 81.82 | 24.16 |
| 77A | 4.83 | 1000 | 86.66 | 30.14 |
| 77B | 19.4 | 1000 | 79.94 | 26.15 |
| 78 | 32.8 | 1000 | 69.34 | 43.62 |
| 79 | 34.7 | 1000 | 67.01 | 43.34 |
| 80A | 7.14 | 1000 | 81.31 | 27.51 |
| 80B | 63.2 | 1000 | 72.81 | 24.11 |
| 81 | 2.55 | 1000 | 94.37 | 41.45 |
| 82 | 2.07 | 1000 | 94.92 | 43.74 |
| 83 | 19.1 | 1000 | 69.87 | 46.1 |
| 84 | 8.55 | 1000 | 79.35 | 29.21 |
| 85 | 14.9 | 1000 | 78.99 | 15.01 |
| 86 | 18.2 | 1000 | 65.45 | 35.49 |
| 87 | 5.59 | 1000 | 88.43 | 23.14 |
| 88 | 11.3 | 1000 | 81.32 | 33.19 |
| 89 | 4.81 | 1000 | 92.15 | 37.98 |
| 90 | 2.77 | 1000 | 89.33 | 28.96 |
| 91 | 4.35 | 1000 | 86.50 | 36.18 |
| 92 | 4.55 | 1000 | 89.69 | 21.9 |
| 93 | 2.08 | 1000 | 93.98 | 43.19 |
| 94 | 5.62 | 1000 | 89.44 | 23.5 |
| 95 | 2.26 | 1000 | 89.17 | 40.77 |
| 96 | 3.15 | 1000 | 88.39 | 35.42 |
| 97 | 4.9 | 1000 | 86.15 | 33.51 |
| 98 | 10.2 | 1000 | 85.36 | 8.51 |
| 99 | 0.22 | 4.48 | 97.89 | 86.57 |
| 100 | 8.78 | 1000 | 85.43 | 21.43 |
| 101 | 0.77 | 43.4 | 95.72 | 60.66 |
| 102 | 5.34 | 1000 | 83.27 | 37.55 |
| 103 | 3.24 | 1000 | 90.68 | 40.75 |
| 104 | 0.23 | 19.9 | 98.71 | 75.21 |
| 105 | 3.28 | 1000 | 89.59 | 44.72 |
| 106 | 2.76 | 1000 | 93.31 | 29.86 |
| 107 | 5.08 | 1000 | 86.52 | 22.64 |
| 108 | 8.12 | 1000 | 83.17 | 7.19 |
| 109 | 1.12 | 39.6 | 95.44 | 61.94 |
| 110 | 5.42 | 1000 | 91.74 | 46.21 |
| 111 | 4.1 | 1000 | 88.4 | 35.88 |
| 112 | 2.65 | 99.7 | 96.1 | 56.27 |
| 113 | 2.34 | 1000 | 94.01 | 32.76 |
| 114 | 17.4 | 10000 | 79.9 | 31.29 |
| 115 | 18.2 | 10000 | 77.39 | 38.63 |
| 116 | 1.17 | 1000 | 95.2 | 48.18 |
| 117 | 0.8 | 107 | 97.55 | 55.77 |
| 118 | 7.04 | 1000 | 90.74 | 22.21 |
| 119 | 4.88 | 1000 | 87.76 | 21.95 |
| 120 | 7.06 | 1000 | 90.52 | 23.11 |
| 121 | 3.42 | 1000 | 88.82 | 45.94 |
| 122 | 2.1 | 1000 | 93.15 | 48.34 |
| 123 | 1.81 | 1000 | 92.49 | 33.94 |
| 124 | 1.36 | 108 | 95.37 | 52.87 |
| 125 | 3.54 | 1000 | 89.56 | 14.78 |
| 126 | 2.71 | 142 | 95.31 | 57.57 |
| 127 | 5.21 | 1000 | 82.02 | 35.91 |
| 128 | 2.41 | 111 | 92.23 | 53.09 |
| 129 | 3.84 | 1000 | 87.14 | 39.57 |
| 130 | 7.5 | 1000 | 82.14 | 35.36 |
| 131 | 11.7 | 1000 | 71.18 | 24.33 |
| 132 | 1.38 | 161 | 93.22 | 51.61 |
| 133 | 3.6 | 237 | 88.45 | 51.62 |
| 134 | 22 | 1000 | 78.39 | 48.21 |
| 135 | 6.19 | 1000 | 87.54 | 49.51 |
| 136 | 7.39 | 135 | 88.35 | 60.64 |
| 137 | 6.75 | 1000 | 85.41 | 46.3 |
| 138 | 3.94 | 53.2 | 92.1 | 67.81 |
| 139 | 7.3 | 1000 | 84.03 | 19.03 |
| 140 | 1.73 | 1000 | 93.1 | 35.38 |
| 141 | 3.3 | 1000 | 94.65 | 39.71 |
| 142 | 9.72 | 1000 | 82.64 | 22.87 |
| 143 | 7.01 | 1000 | 89.76 | 18.45 |
| 144 | 11.4 | 1000 | 76.67 | 10.91 |
| 145 | 28.4 | 1000 | 63.39 | 22.82 |
| 146 | 6.34 | 1000 | 88.1 | 23.42 |
| 147 | 3.87 | 1000 | 87.72 | 25.14 |
| 148 | 1.81 | 1000 | 91.91 | 35.99 |
| 149 | 1.51 | 1000 | 93.65 | 37.67 |
| 150 | 7.15 | 1000 | 86.89 | 24.54 |
| 151 | 3.31 | 113 | 90.35 | 53.04 |
| 152 | 10.1 | 1000 | 86.67 | 23.65 |
| 153 | 0.98 | 25.3 | 96.96 | 72.69 |
| 154 | 0.9 | 25.9 | 97.57 | 77.06 |
| 155 | 26.1 | 1000 | 87.46 | 39.17 |
| 156 | 4.8 | 90.6 | 93.68 | 46.94 |
| 157 | 3.3 | 1000 | 92.81 | 49.64 |
| 158 | 1.72 | 81.3 | 96.27 | 63.57 |
| 159 | 2.4 | 351 | 96.09 | 53.12 |
| 160 | 15.1 | 1000 | 73.21 | 45.29 |
| 161 | 2.68 | 1000 | 92.38 | 45.54 |
| 162 | 6.37 | 1000 | 90.22 | 17.75 |
| 163 | 23 | 1000 | 70.29 | 2.13 |
| 164 | 18.3 | 1000 | 71.95 | 35.07 |
| 165 | 16.1 | 1000 | 78.45 | 28.37 |
| 166 | 0.3 | 28.3 | 98.27 | 74.19 |
| 167A | 2.67 | 1000 | 87.71 | 25.39 |
| 167B | 6.04 | 1000 | 84.17 | 12.12 |
| 168 | 7.58 | 1000 | 88.58 | 15.04 |
| 169 | 5.85 | 1000 | 85.81 | 15.13 |
| 170 | 23 | 1000 | 84.53 | 15.1 |
| 171 | 5.12 | 1000 | 91.49 | 20.15 |
| 172 | 19.7 | 1000 | 77.96 | 19.62 |
| 173 | 19.2 | 1000 | 69.23 | 11.86 |
| 174 | 1000 | 1000 | 49.13 | 19.37 |
| 175 | 2.18 | 10000 | 93.42 | 46.52 |
| 176 | 4.17 | 10000 | 86.45 | 52.04 |
| 177 | 6.32 | 10000 | 82.46 | 44.28 |
| 178 | 6.22 | 1000 | 86.51 | 39.52 |
| 179 | 5.11 | 1000 | 90.38 | 38.38 |
| 180 | 7.35 | 41.3 | 82.68 | 61.8 |
| 181 | 14.8 | 10000 | 68.53 | 26.03 |
| 182 | 15.6 | 10000 | 81.32 | 45.53 |
| 183 | 15.7 | 10000 | 71.44 | 15.91 |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A compound having a structure represented by Formula (II): 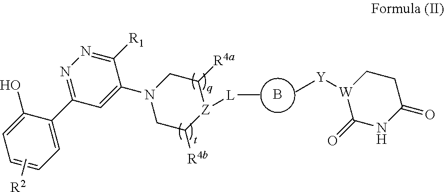
or a stereoisomer, pharmaceutically acceptable salt thereof, wherein:
t and q is independently 0 or 1;
Z is —N— or —C(R³)—;
R¹ is hydrogen or C₁₋₆ alkyl optionally substituted with 1 to 3 R^{a};
R² is hydrogen, halo, hydroxyl, —O—R^{c}, C₁₋₆ alkyl optionally substituted with 1 to 3 R^{a};
R³ is hydrogen, —CN, halo (preferably F), —O—R^{e}, C₁₋₆ alkyl, C₆₋₁₂ aryl, C₃₋₁₂ cycloalkyl, 5-12 membered heteroaryl, wherein each of C₁₋₆ alkyl, C₆₋₁₂ aryl, C₃₋₁₂ cycloalkyl, and 5-12 membered heteroaryl is optionally substituted with 1 to 3 R^{d};
R^{4a} and R^{4b} are each independently hydrogen, C₁₋₆ alkyl or C₁₋₆ alkoxy, or
R^{4a} and R^{4b} together may form a C₁₋₃ alkylene chain; or
R^{4a} and R³ together with the carbons to which they are attached form a C₃₋₁₂ cycloalkyl or 5-12 membered heterocycle,
L comprises up to 8 linker segments represented by -L₁-L₂-L₃-L₄-L₅-L₆-L₇-L₈-, each L₁, L₂, L₃, L₄, L₅, L₆, L₇, or L₈, being independently:
i) C₃₋₁₂ cycloalkyl optionally substituted with 1-3 R^{b};
ii) C₆₋₁₂ aryl optionally substituted with 1-3 R^{b};
iii) 4-12 membered heterocyclyl optionally substituted with 1-3 R^{b};
iv) 5-12 membered heteroaryl optionally substituted with 1-3 R^{b};
v) direct bond;
vi) C₁₋₁₂ alkylene chain optionally substituted with 1-3 R^{b};
vii) C₁₋₁₂ alkenylene chain optionally substituted with 1-3 R^{b}; or
viii) —(CH₂)ₘ—C(O)—, —(CH₂)ₘ—C(O)O—, —(CH₂)ₘ—O—, —(CH₂)ₘ—N(R^{c})—, —(CH₂)ₘ—S—, —(CH₂)ₘ—C(S)—, —(CH₂)ₘ—C(S)—O—, —(CH₂)ₘ—S(O)₂—, —(CH₂)ₘ—S(O)═N—, —(CH₂)ₘ—S(O)₂NH—, —(CH₂)ₘ—C(O)—N(R^{c})—, —C(O)—N(R^{c})—(CH₂)ₘ—, —(CH₂)ₘ—O—C(O)—N(R^{c})—, —(CH₂)ₘ—O—C(O)—O—, or —NH—(CH₂)ₘ—C(O)—, wherein m is 0, 1, 2, 3, 4, 5 or 6;
each R^{a} is independently halo, or —O—R^{c};
each R^{b} is independently oxo, imino, sulfoximino, halo, nitro, —CN, C₁₋₆ alkyl, C₂₋₆ alkenyl, C₃₋₁₅ cycloalkyl, C₁₋₈ haloalkyl, C₆₋₁₂ aryl, 5-12 membered heteroaryl, 4-12 membered heterocyclyl, —O—R^{c}, —C(O)—R^{c}, —C(O)O—R^{c}, —C(O)—N(R^{c})(R^{c}), —N(R^{c})(R^{c}), —N(R^{c})C(O)—R^{c}, —N(R^{c})C(O)O—R^{c}, —N(R^{c})C(O)N(R^{c})(R^{c}), —N(R^{c})S(O)₂(R^{c}), —NR^{c}S(O)₂N(R^{c})(R^{c}), —N(R^{c})S(O)₂O(R^{c}), —OC(O)R^{c}, —OC(O)—N(R^{c})(R^{c}), —Si(R^{c})₃, —S—R^{c}, —S(O)R^{c}, —S(O)(NH)R^{c}, —S(O)₂R^{c} or —S(O)₂N(R^{c})(R^{c}), wherein each of C₁₋₆ alkyl, C₂₋₆ alkenyl, C₃₋₁₅ cycloalkyl, C₁₋₈ haloalkyl, C₆₋₁₂ aryl, 5-12 membered heteroaryl, and 4-12 membered heterocyclyl may be optionally substituted with 1 to 3 R^{d};
each R^{c} is independently hydrogen, C₁₋₆ alkyl, C₁₋₆ alkoxy, C₃₋₆ cycloalkyl (optionally substituted with 1 or 2 halo, C₁₋₃ alkyl or C₁₋₃ alkoxy), C₆₋₁₂ aryl, 4-12 membered heterocyclyl optionally substituted with 1 or 2 C₁₋₃ alkyl), 5-12 membered heteroaryl optionally substituted with 1 or 2 C₁₋₃ alkyl or C₁₋₃ haloalkyl) or C₁₋₆ haloalkyl, wherein C₁₋₆ alkyl is optionally substituted with one or more substituents selected from C₁₋₃ alkoxy, hydroxy, C₃₋₆ cycloalkyl (optionally further substituted with 1 or 2 halo, C₁₋₃ alkoxy or C₁₋₃ alkyl), CN, 5-12 membered heteroaryl (optionally further substituted with 1 or 2 halo or C₁₋₃ alkyl), C₆₋₁₂ aryl and amino;
each R^{d} is independently halo, —CN, —O—R^{c}, C₁₋₆ alkyl, C₆₋₁₂ aryl, C₃₋₆ cycloalkyl or C₁₋₆ haloalkyl;
R^{e} is C₁₋₆ alkyl, C₃₋₁₂ cycloalkyl, C₆₋₁₂ aryl, 5-12 membered heteroaryl, each being optionally substituted with C₁₋₆ alkyl, C₁₋₆ alkoxy, C₁₋₆ haloalkyl or halo;
W is —C(R^{g})— or —N—;
Y is direct bond, C₁₋₄ alkylene chain, —C(O)—, —C(O)O—, —O—, —N(R^{g})—, —S— —C(S)—, —C(S)—O—, —O—C(O)O—, —C(O)—N(R^{g})—, or —O—C(O)—N(R^{g})—;
B ring is C₆₋₁₂ aryl, 5-12 membered heteroaryl, or 4-12 membered heterocyclyl, each being optionally substituted with 1 to 3 R^{j};
R^{g} is hydrogen or C₁₋₆ alkyl; and
each R^{j} is independently halo, oxo, —CN, —O—R^{c}, C₁₋₆ alkyl, or C₁₋₆ haloalkyl.

2. A compound having a structure represented by Formula (I): 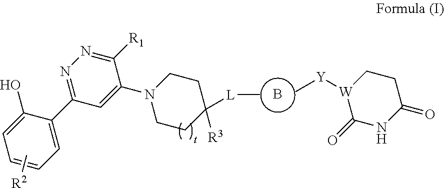
or a stereoisomer, pharmaceutically acceptable salt thereof, wherein:
t is 0 or 1;
R¹ is hydrogen or C₁₋₆ alkyl optionally substituted with 1 to 3 R^{a};
R² is hydrogen, halo, hydroxyl, —O—R^{c}, C₁₋₆ alkyl optionally substituted with 1 to 3 R^{a};
R³ is hydrogen, —CN, C₁₋₆ alkyl, C₆₋₁₂ aryl, C₃₋₁₂ cycloalkyl, 5-12 membered heteroaryl, each being optionally substituted with 1 to 3 R^{d}, or —O—R^{e};
L comprises up to 8 linker segments represented by -L₁-L₂-L₃-L₄-L₅-L₆-L₇-L₈-, each L₁, L₂, L₃, L₄, L₅, L₆, L₇, or L₈, being independently:
i) C₃₋₁₂ cycloalkyl optionally substituted with 1-3 R^{b};
ii) C₆₋₁₂ aryl optionally substituted with 1-3 R^{b};
iii) 4-12 membered heterocyclyl optionally substituted with 1-3 R^{b};
iv) 5-12 membered heteroaryl optionally substituted with 1-3 R^{b};
v) direct bond;
vi) C₁₋₁₂ alkylene chain optionally substituted with 1-3 R^{b}; or
vii) —(CH₂)ₘ—C(O)—, —(CH₂)ₘ—C(O)O—, —(CH₂)ₘ—O—, —(CH₂)ₘ—N(R^{c})—, —(CH₂)ₘ—S—, —(CH₂)ₘ—C(S)—, —(CH₂)ₘ—C(S)—O—, —(CH₂)m—S(O)₂—, —(CH₂)ₘ—S(O)═N—, —(CH₂)ₘ—S(O)₂NH—, —(CH₂)ₘ—C(O)—N(R^{c})—, —C(O)—N(R^{c})—(CH₂)ₘ—, —(CH₂)ₘ—O—C(O)—N(R^{c})—, —(CH₂)ₘ—O—C(O)—O—, or —NH—(CH₂)ₘ—C(O)—, wherein m is 0, 1, 2, 3, 4, 5 or 6;
each R^{a} is independently halo, or —O—R^{c};
each R^{b} is independently oxo, imino, sulfoximino, halo, nitro, —CN, C₁₋₆ alkyl, C₂₋₆ alkenyl, C₃₋₁₅ cycloalkyl, C₁₋₈ haloalkyl, C₆₋₁₂ aryl, 5-12 membered heteroaryl, 4-12 membered heterocyclyl, —O—R^{c}, —C(O)—R^{c}, —C(O)O—R^{c}, —C(O)—N(R^{c})(R^{c}), —N(R^{c})(R^{c}), —N(R^{c})C(O)—R^{c}, —N(R^{c})C(O)O—R^{c}, —N(R^{c})C(O)N(R^{c})(R^{c}), —N(R^{c})S(O)₂(R^{c}), —NR^{c}S(O)₂N(R^{c})(R^{c}), —N(R^{c})S(O)₂O(R^{c}), —OC(O)R^{c}, —OC(O)—N(R^{c})(R^{c}), —Si(R^{c})₃, —S—R^{c}, —S(O)R^{c}, —S(O)(NH)R^{c}, —S(O)₂R^{c} or —S(O)₂N(R^{c})(R^{c}), wherein each of C₁₋₆ alkyl, C₂₋₆ alkenyl, C₃₋₁₅ cycloalkyl, C₁₋₈ haloalkyl, C₆₋₁₂ aryl, 5-12 membered heteroaryl, and 4-12 membered heterocyclyl may be optionally substituted with 1 to 3 R^{d};
each R^{c} is independently hydrogen, C₁₋₆ alkyl, C₁₋₆ alkoxy, C₃₋₆ cycloalkyl, C₆₋₁₂ aryl, or C₁₋₆ haloalkyl, wherein C₁₋₆ alkyl is optionally substituted with one or more substituents selected from C₁₋₃ alkoxy, hydroxy, C₃₋₆ cycloalkyl, C₆₋₁₂ aryl and amino;
each R^{d} is independently halo, —CN, —O—R^{c}, C₁₋₆ alkyl, C₆₋₁₂ aryl, or C₁₋₆ haloalkyl;
R^{e} is C₁₋₆ alkyl, C₃₋₁₂ cycloalkyl, C₆₋₁₂ aryl, 5-12 membered heteroaryl, each being optionally substituted with C₁₋₆ alkyl, C₁₋₆ haloalkyl or halo;
W is —C(R^{g})— or —N—;
Y is direct bond, C₁₋₄ alkylene chain, —C(O)—, —C(O)O—, —O—, —N(R^{g})—, —S— —C(S)—, —C(S)—O—, —O—C(O)O—, —C(O)—N(R^{g})—, or —O—C(O)—N(R^{g})—;
B ring is C₆₋₁₂ aryl, 5-12 membered heteroaryl, or 4-12 membered heterocyclyl, each being optionally substituted with 1 to 3 R^{j};
R^{g} is hydrogen or C₁₋₆ alkyl; and
each R^{j} is independently halo, oxo, —CN, —O—R^{c}, C₁₋₆ alkyl, or C₁₋₆ haloalkyl.

3. The compound of claim 2, wherein t is 1, q is 1, and the compound has the following structure:

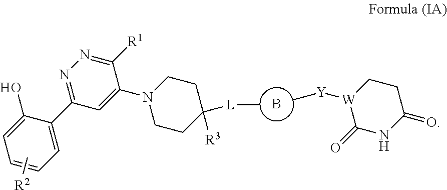

4. The compound of claim 2, wherein t is 0, q is 1, and the compound has the following structure:

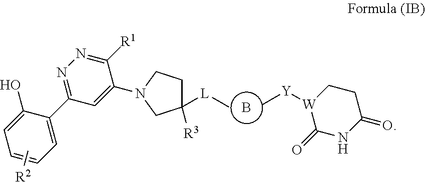

5. The compound of claim 1, wherein t is 1, q is 1, R^{4a} and R^{4b} together form a C₁₋₃ alkylene chain, and the compound has one of the following structures:

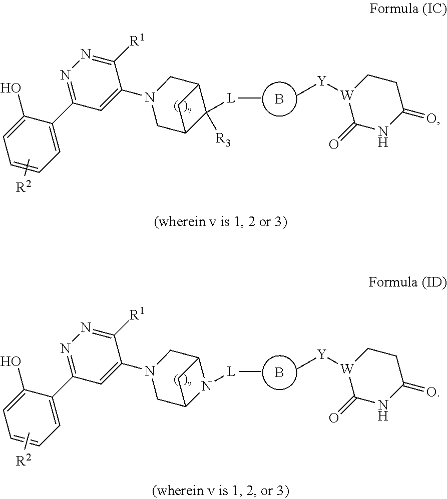

6. The compound of claim 1, wherein t is 0, q is 1, and R^{4a} and R³ together form a methylene chain, and the compound has the following structure:

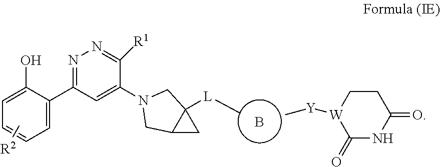

7. The compound of claim 1, wherein t is 0, and q is 0, and the compound has the following structure:

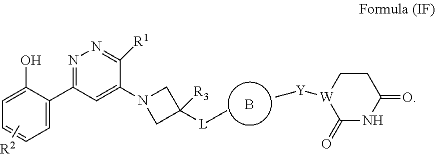

8. The compound of claim 1, wherein each L₁, L₂, L₃, L₄, L₅, L₆, L₇, or L₈ is independently:
i) a bivalent ring moiety selected from the group consisting of: 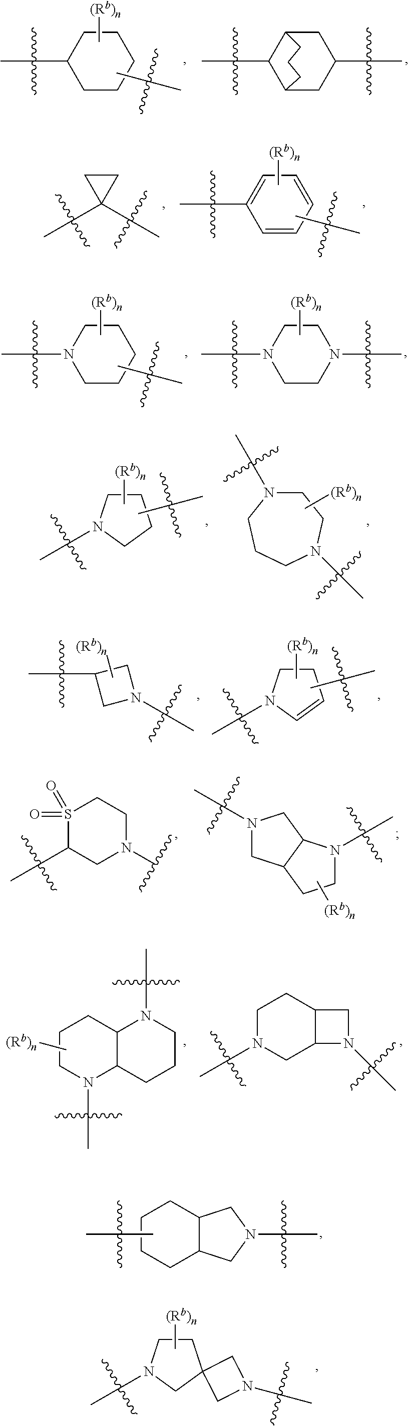 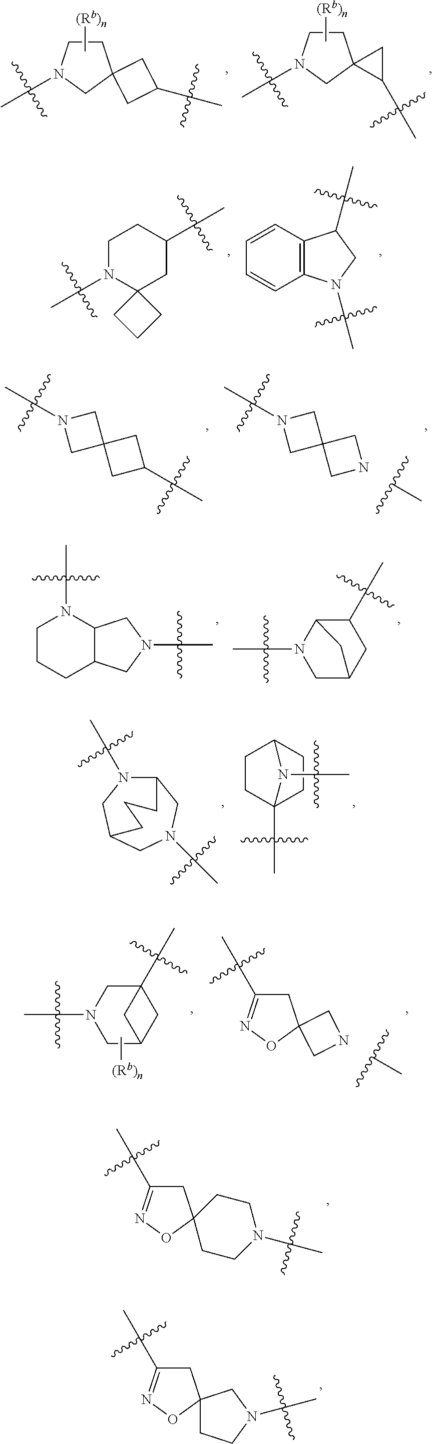 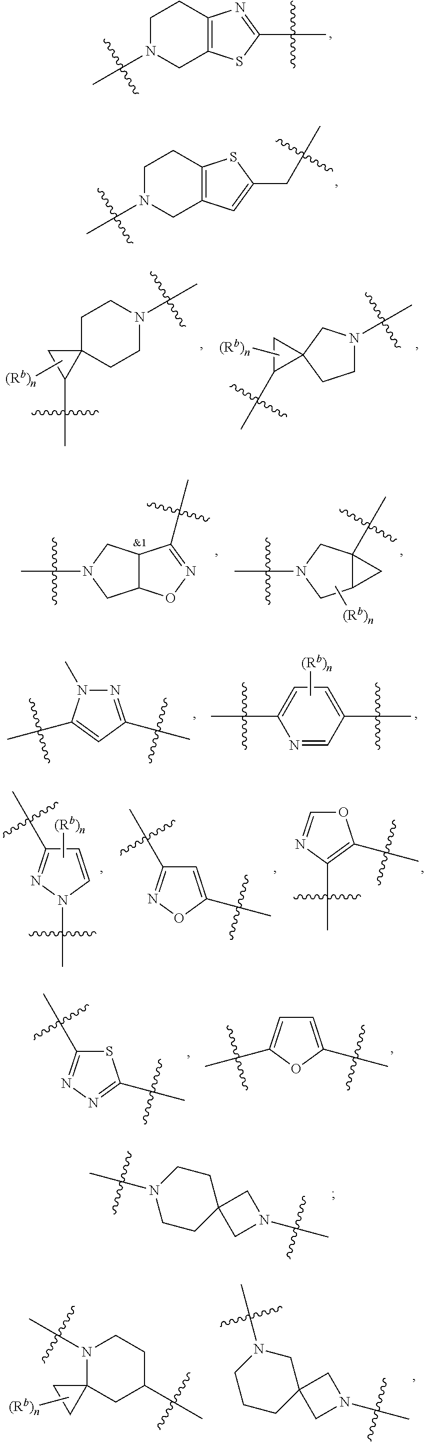 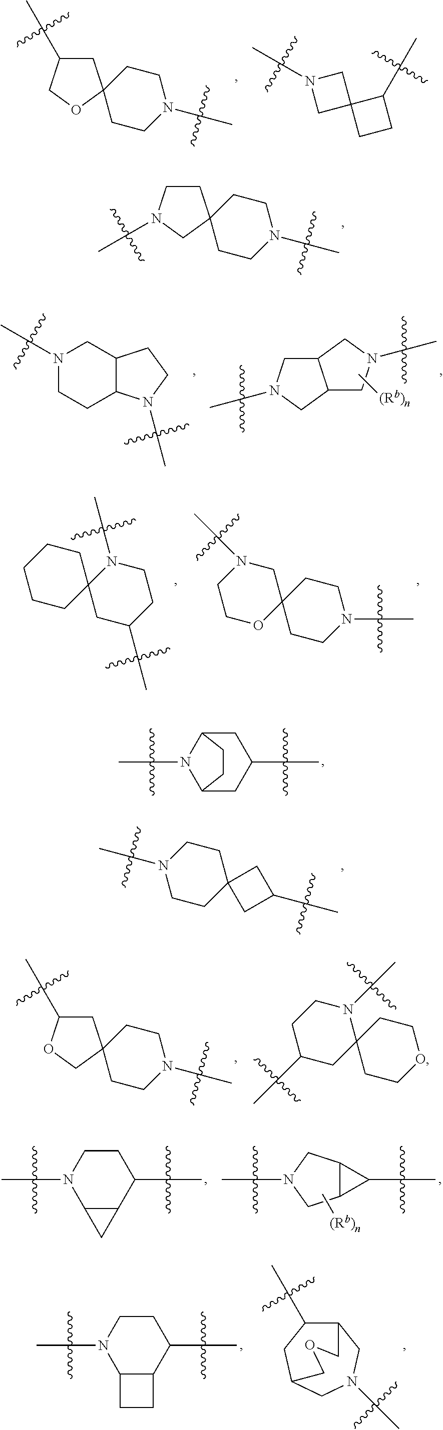 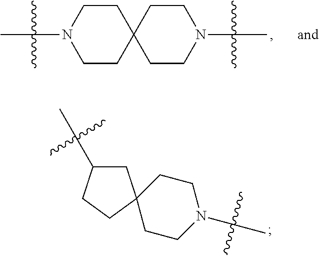 [SMILES: *N1CCC2(CC1)CCN(*)CC2 ; *C1CCC2(CCN(*)CC2)C1]
ii) direct bond;
iii) C₁₋₆ alkylene chain optionally substituted with 1-3 R^{b}; or
iv) —C(O)—, —O—, —(CH₂)ₘ—C(O)—N(R^{c})—, —(CH₂)ₘ—C(O)—, —(CH₂)ₘ—N(R^{c})—, —C(O)—N(R^{c})—(CH₂)ₘ—, or —NH—(CH₂)ₘ—C(O)—, —(CH₂)ₘ—S(O)₂NH—, m being 0, 1, 2 or 3;
wherein,
n is 0, 1, or 2;
R^{b} is halo, —O—R, —CN, C₁₋₆ alkyl, or C₁₋₆ haloalkyl; and
R^{c} is hydrogen, C₁₋₆ alkyl, C₁₋₆ alkoxy, C₃₋₆ cycloalkyl, phenyl, or C₁₋₆ haloalkyl, wherein C₁₋₆ alkyl is optionally substituted with C₁₋₃ alkoxy, hydroxy, C₃₋₆ cycloalkyl, phenyl and amino.

9. The compound of claim 1, wherein each L₁, L₂, L₃, L₄, L₅, L₆, L₇, or L₈ is independently:
i) a bivalent ring moiety selected from the group consisting of: 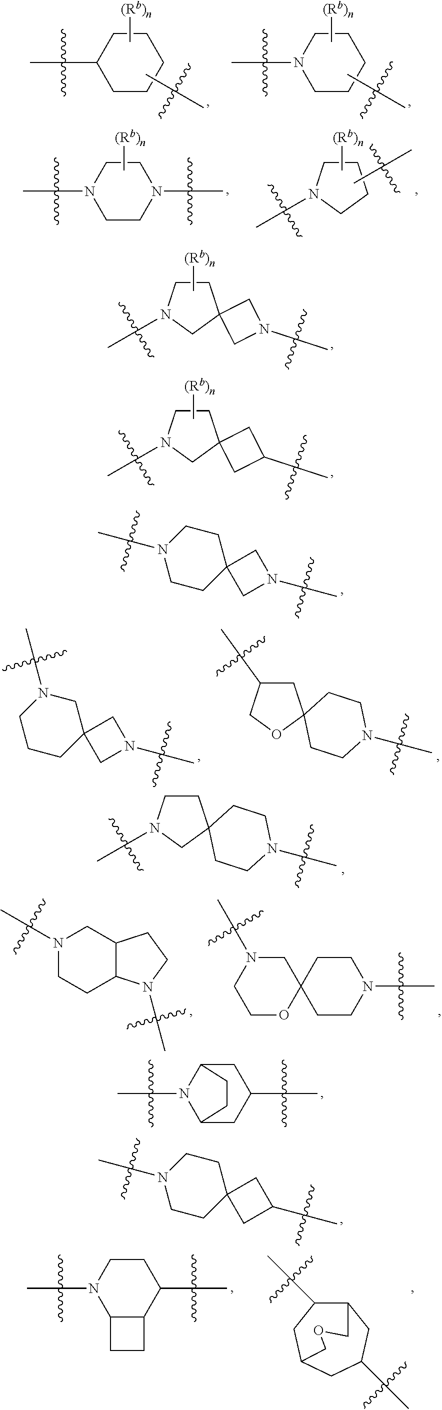 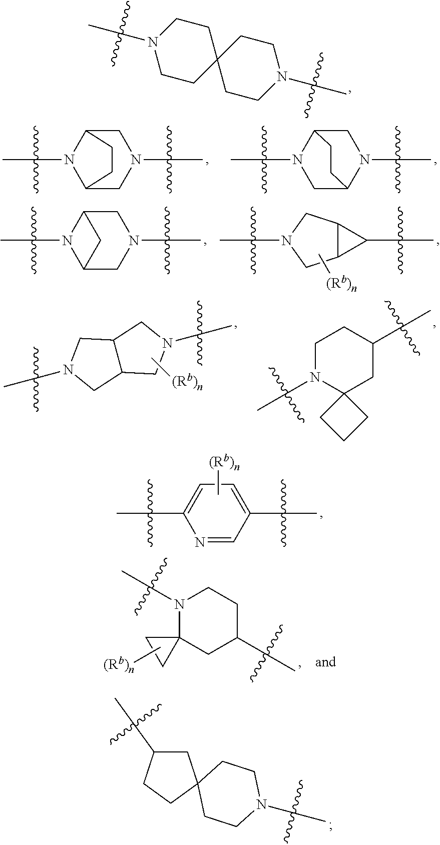
ii) direct bond;
iii) C₁₋₆ alkylene chain optionally substituted with 1-3 R^{b};
iv) C₁₋₆ alkenylene chain optionally substituted with 1-3 R^{b}; or
v) —C(O)—, —O—, —(CH₂)ₘ—C(O)—N(R^{c})—, —(CH₂)ₘ—C(O)—, —(CH₂)ₘ—N(R^{c})—, —C(O)—N(R^{c})—(CH₂)ₘ—, or —NH—(CH₂)ₘ—C(O)—, —(CH₂)ₘ—S(O)₂NH—, m being 0, 1, 2 or 3;
wherein,
n is 0, 1, or 2;
R^{b} is halo, —O—R, —CN, C₁₋₆ alkyl, or C₁₋₆ haloalkyl; and
wherein R^{c} is hydrogen, C₁₋₆ alkyl, C₁₋₆ alkoxy, C₁₋₆ haloalkyl, C₃₋₆ cycloalkyl (optionally substituted with 1 or 2 halo, C₁₋₃ alkyl or C₁₋₃ alkoxy), 4-12 membered heterocyclyl (optionally substituted with 1 or 2 C₁₋₃ alkyl), or 5-12 membered heteroaryl (optionally substituted with 1 or 2 C₁₋₃ alkyl or C₁₋₃ haloalkyl), wherein C₁₋₆ alkyl is optionally substituted with 1 or 2 substituents selected from the group consisting of C₁₋₃ alkoxy, CN, 5-12 member heteroaryl (optionally further substituted with 1 or 2 halo or C₁₋₃ alkyl), hydroxy, C₃₋₆ cycloalkyl (optionally further substituted with 1 or 2 halo, C₁₋₃ alkoxy or C₁₋₃ alkyl), phenyl and amino.

10. The compound of claim 1, wherein,
Y is direct bond,—C(O)—N(R^{g})— (wherein R^{g} is hydrogen or C₁₋₆ alkyl), or —NH—; and
B ring is: 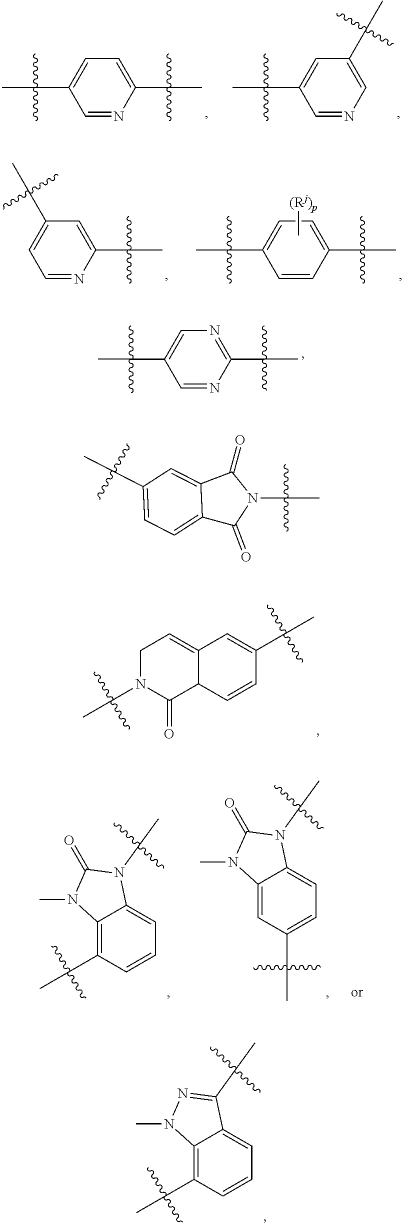
wherein p is 0, 1 or 2, R^{j} is halo, —CN, —O—R^{c}, C₁₋₆ alkyl, or C₁₋₆ haloalkyl.

11. The compound of claim 1, wherein R¹ is hydrogen and R² is hydrogen, halo or C₁₋₃ alkyl.

12. (canceled)

13. The compound of claim 1, wherein R¹ is hydrogen, L₆, L₇ and L₈ are direct bonds, and the compound has the following structure: 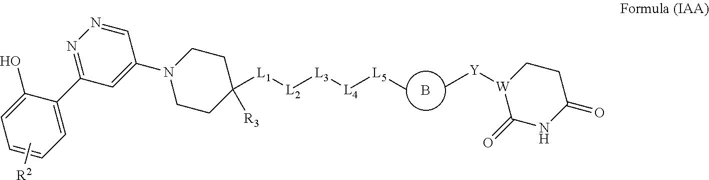
wherein,
R² is hydrogen, halo or C₁₋₃ alkyl;
R³ is halo, C₃₋₆ cycloalkyl, C₆₋₁₂ aryl, —O—R^{e} (wherein R^{e} is C₃₋₆ cycloalkyl or C₁₋₆ alkyl optionally substituted with one or more C₁₋₃ alkoxy or halo), C₁₋₆ alkyl optionally substituted with C₁₋₃ alkoxy, C₆₋₁₂ aryl, halo, or 5-12 membered heteroaryl optionally substituted with 1 or 2 C₁₋₆ alkyl or C₁₋₆ haloalkyl;
L₁ is —C(O)— or —C(O)—N(R^{c})—, wherein R^{c} is hydrogen, C₁₋₆ alkyl, C₁₋₆ alkoxy, C₁₋₆ haloalkyl, C₃₋₆ cycloalkyl (optionally substituted with 1 or 2 halo, C₁₋₃ alkyl or C₁₋₃ alkoxy), 4-12 membered heterocyclyl (optionally substituted with 1 or 2 C₁₋₃ alkyl), or 5-12 membered heteroaryl (optionally substituted with 1 or 2 C₁₋₃ alkyl or C₁₋₃ haloalkyl), wherein C₁₋₆ alkyl is optionally substituted with 1 or 2 substituents selected from the group consisting of C₁₋₃ alkoxy, CN, 5-12 member heteroaryl (optionally further substituted with 1 or 2 halo or C₁₋₃ alkyl), hydroxy, C₃₋₆ cycloalkyl (optionally further substituted with 1 or 2 halo, C₁₋₃ alkoxy or C₁₋₃ alkyl), phenyl and amino;
L₂ is 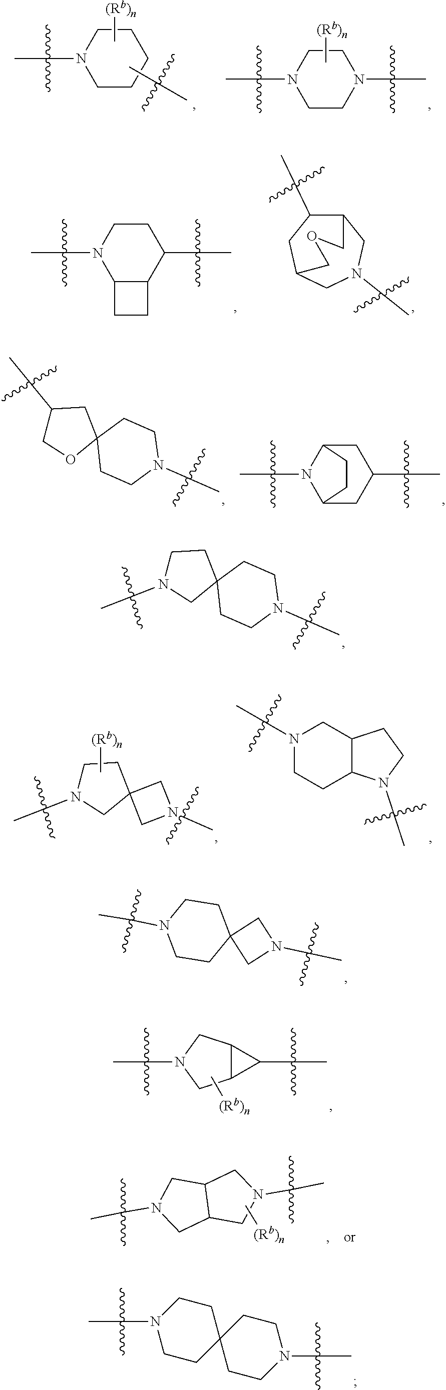
L₃ is direct bond, —CH₂—C(O)—, —(CH₂)₂—O—, or C₁₋₄ alkylene chain optionally substituted with C₁₋₃ alkyl or C₂₋₄ alkylene chain optionally substituted with C₁₋₃ alkyl;
L₄ is 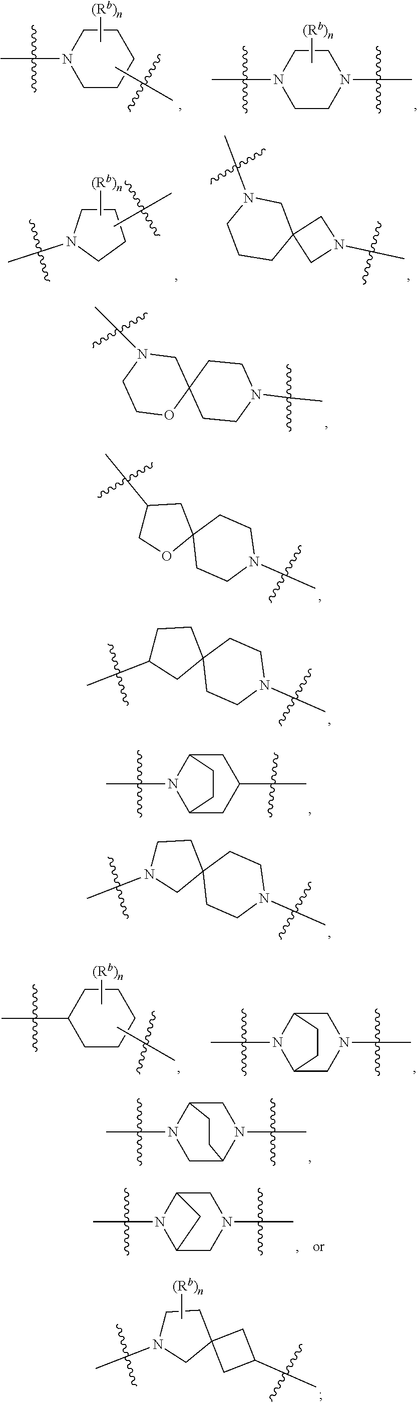
n is 0, 1 or 2, R^{b} is halo, C₁₋₃ alkyl, or C₁₋₃ alkoxy;
L₅ is direct bond or —O—;
B is 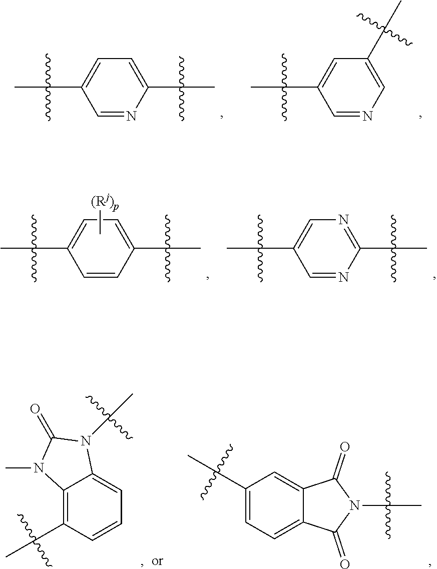
wherein p is 0, 1, or 2, R^{j} is halo;
Y is direct bond, or —C(O)—N(Me)-; and
W is CH or N.

14. The compound of claim 2, wherein R¹ is hydrogen, L₆, L₇ and L₈ are direct bonds, and the compound has the structure represented by Formula (IAA): 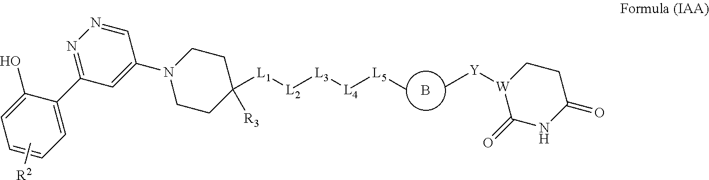
wherein,
R² is hydrogen, halo or C₁₋₃ alkyl;
R³ is —O—R^{e} (wherein R^{e} is C₃₋₆ cycloalkyl or C₁₋₆ alkyl), C₁₋₆ alkyl optionally substituted with C₁₋₃ alkoxy, 5-12 membered heteroaryl optionally substituted with 1 or 2 C₁₋₆ alkyl, or C₁₋₆ haloalkyl;
L₁ is —C(O)— or —C(O)—N(R^{c})—, wherein R^{c} is hydrogen, C₁₋₆ alkyl, C₁₋₆ alkoxy, C₃₋₆ cycloalkyl, or C₁₋₆ haloalkyl, wherein C₁₋₆ alkyl is optionally substituted with 1 or 2 C₁₋₃ alkoxy, hydroxy, C₃₋₆ cycloalkyl (optionally substituted with C₁₋₃ alkoxy), phenyl or amino;
L₂ is 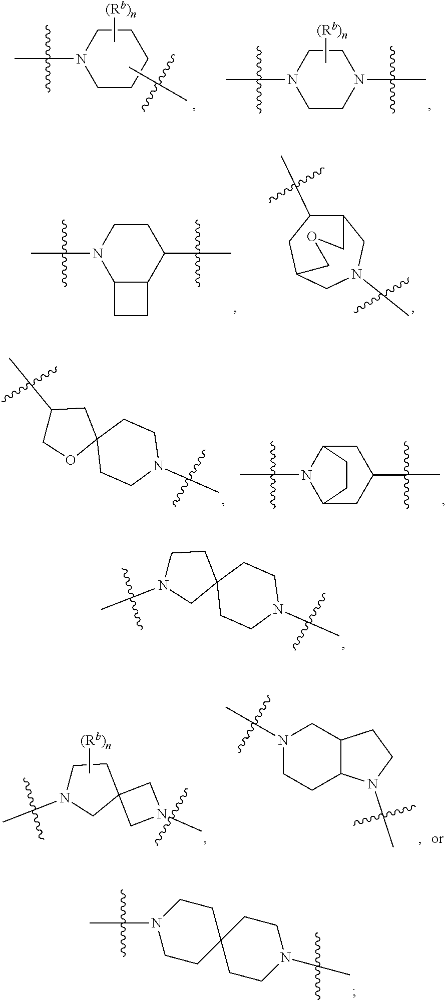
L₃ is direct bond, —CH₂—C(O)— or C₁₋₂ alkylene chain optionally substituted with C₁₋₃ alkyl;
L₄ is 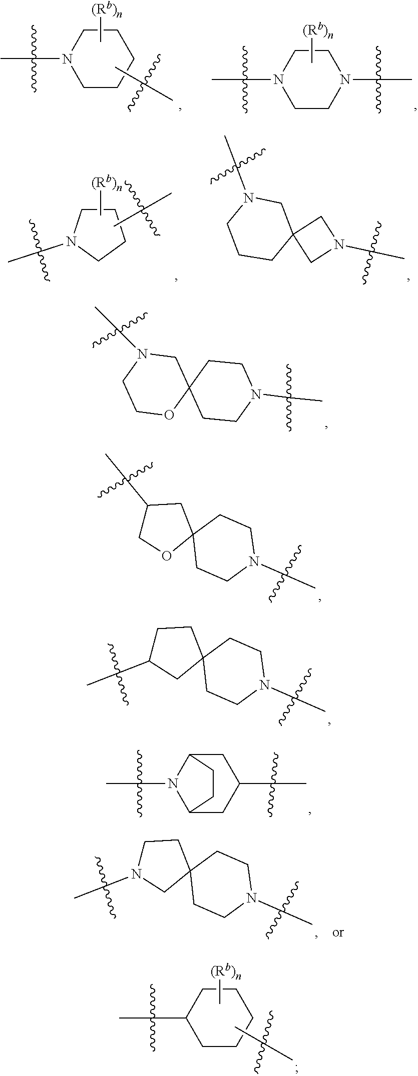
n is 0, 1 or 2, R^{b} is halo or C₁₋₃ alkyl;
L₅ is direct bond or —O—;
B is 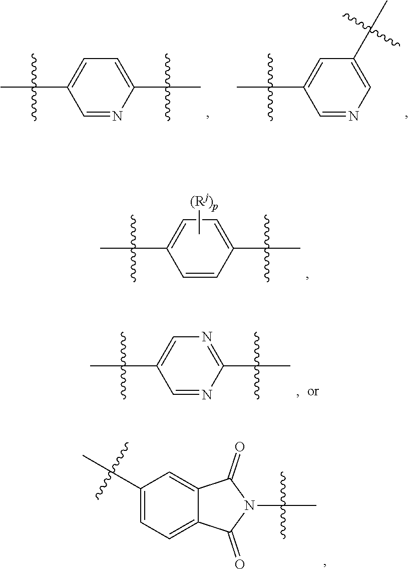
wherein p is 0, 1, or 2, R³ is halo;
Y is direct bond, or —C(O)—N(Me)-; and
W is CH or N.

15. The compound of claim 14, wherein Y is direct bond, W is CH, and the compound has the following structure:

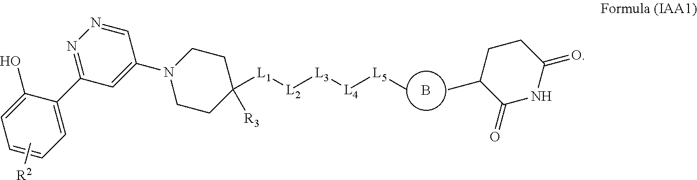

16. The compound of claim 14, wherein,
-L₂-L₃-L₄- has the following structures:

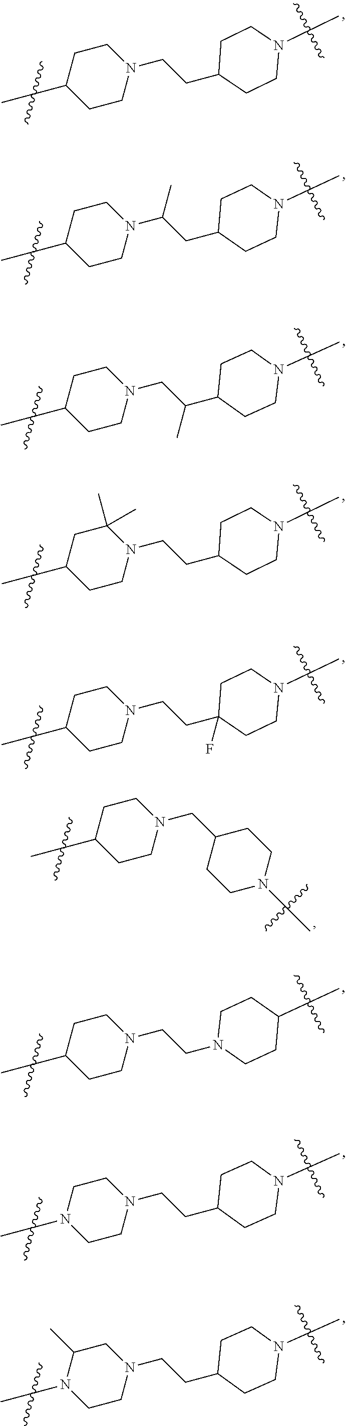

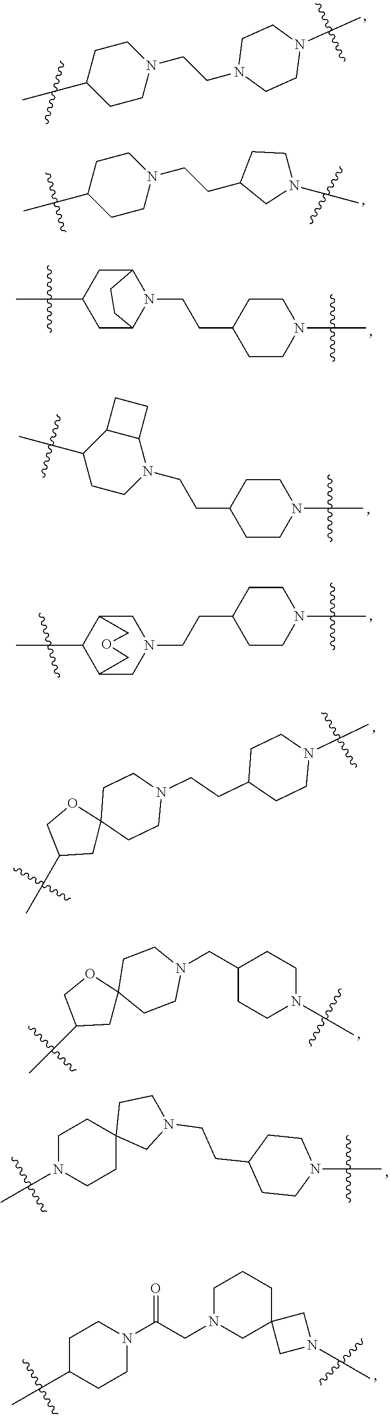

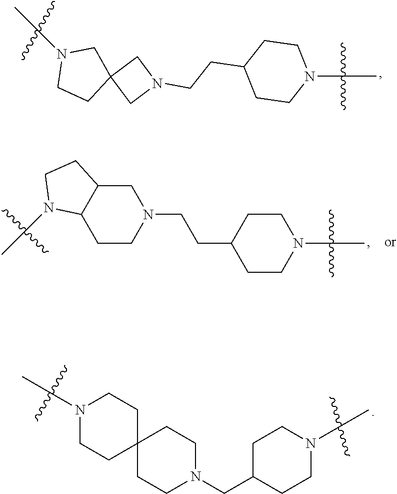 [SMILES: *N1CCC(CCN2CC3(CCN(*)C3)C2)CC1 ; *N1CCC(CCN2CCC3C(CCN3*)C2)CC1 ; *N1CCC(CN2CCC3(CCN(*)CC3)CC2)CC1]

17. The compound of a claim 14, wherein
R² is hydrogen, F, Cl or methyl;
R³ is —O—R^{e} (wherein R^{e} is methyl, ethyl, propyl, cyclopropyl or cyclobutyl), —CH₂OCH₃, thiazol-4yl, thiazol-5yl, 1-methyl-1H-pyrazol-5yl, 1-ethyl-1H-pyrazol-5yl, 1,3-dimethyl-1H-pyrazol-5-yl, 1,4-dimethyl-1H-pyrazol-5-yl;
L₁ is C(O), or —C(O)—N(R^{c})—, wherein R^{c} is hydrogen, methyl, ethyl, propyl, isopropyl, cyclopropyl, cyclobutyl, —OCH₃, —CH₂CF₃, —CH₂CH₂—N(CH₃)₂, —CH₂CH₂—OCH₃, —CH₂-cyclopropyl, benzyl, or 1,3-dihydroxypropan-2yl; and
B is 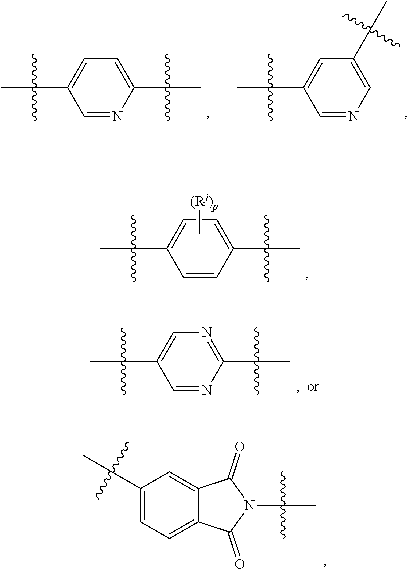
wherein p is 0, 1, or 2, R^{j} is F.

18. The compound of claim 14, wherein
L₃ is a direct bond, and
-L₂-L₃-L₄- has the following structures:

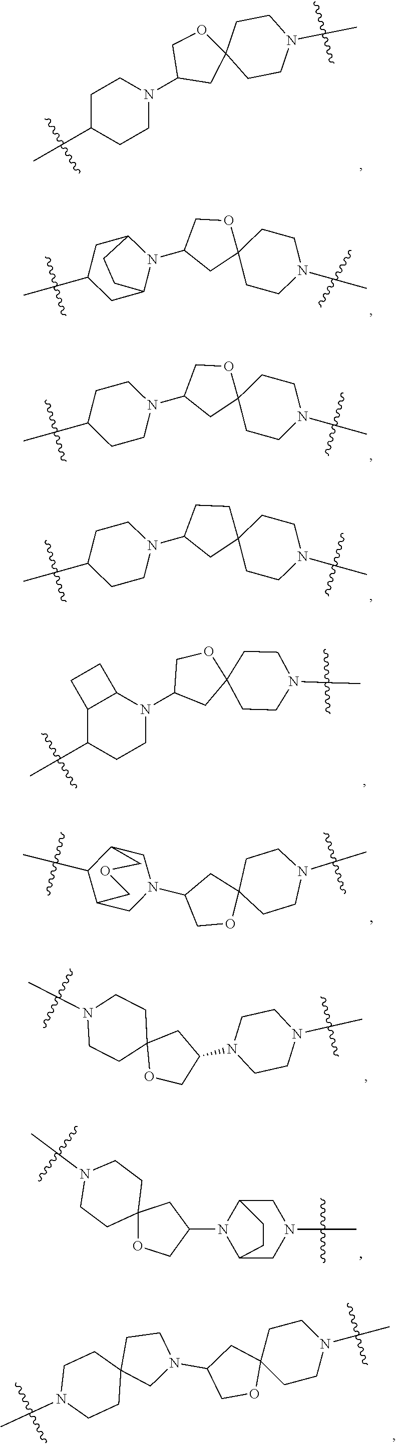

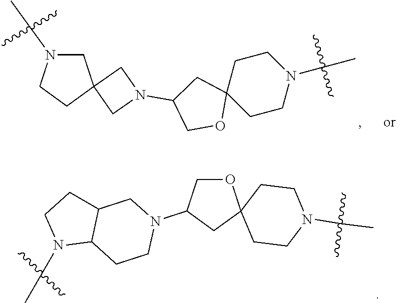 [SMILES: *N1CCC2(CC1)CC(N1CC3(CCN(*)C3)C1)CO2 ; *N1CCC2(CC1)CC(N1CCC3C(CCN3*)C1)CO2]

19. The compound of any one of claim 14, wherein
R² is hydrogen;
R³ is —O—R^{e} (wherein R^{e} is methyl, ethyl, propyl, cyclopropyl or cyclobutyl), thiazol-4yl, thiazol-5yl, 1-methyl-IH-pyrazol-5yl, 1-ethyl-IH-pyrazol-5yl, 1,3-dimethyl-1H-pyrazol-5-yl, 1,4-dimethyl-1H-pyrazol-5-yl, 1-(oxan-4-yl)-1H-pyrazol-5-yl, 1-methyl-3-(trifluoromethyl)-1H-pyrazol-5-yl, or 2-methyl-2H-indazol-3-yl;
L₁ is C(O), or —C(O)—N(R^{c})—, wherein R^{c} is hydrogen, methyl, ethyl, propyl, isopropyl, cyclopropyl, cyclobutyl, —CH₂CF₃, —CH₂CH₂—N(CH₃)₂, —CH₂CH₂—OCH₃, or benzyl; and
B is 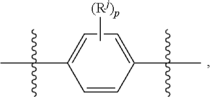
wherein p is 0, 1, or 2, R^{j} is halo.

20. The compound of claim 3, wherein R³ is pyrazol-5yl (optionally substituted with 1 or 2 methyl, trifluoromethyl, oxan-4-yl), thiazolyl, or indazolyl (optionally substituted with 1 or 2 methyl), —CH₂OCH₃, or O-cyclobutyl.

21. The compound of claim 14 having the structure of Formula (IAA2) or Formula (IAA3): 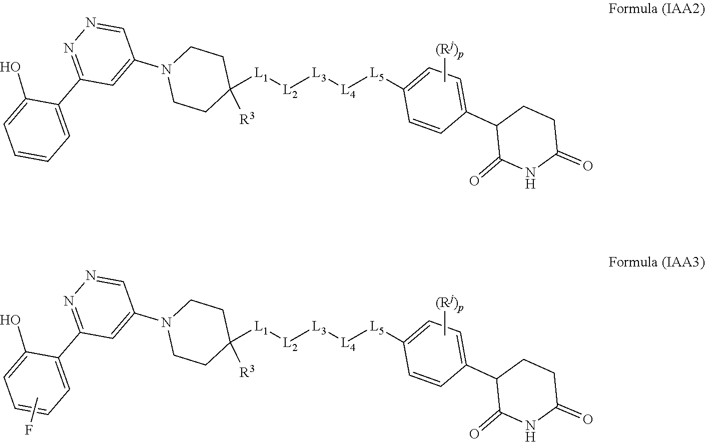
wherein,
R³ is —O—R^{e} (wherein R^{e} is C₃₋₆ cycloalkyl or C₁₋₆ alkyl), C₁₋₆ alkyl optionally substituted with C₁₋₃ alkoxy, or 5-12 membered heteroaryl optionally substituted with 1 or 2 C₁₋₆ alkyl or C₁₋₆ haloalkyl;
L₁ is C(O), or —C(O)—N(R^{c})—, wherein R^{c} is hydrogen, C₁₋₆ alkyl, C₁₋₆ alkoxy, C₃₋₆ cycloalkyl, or C₁₋₆ haloalkyl, wherein C₁₋₆ alkyl is optionally substituted with 1 or 2 C₁₋₃ alkoxy, hydroxy, C₃₋₆ cycloalkyl, phenyl or amino;
-L₂-L₃-L₄-L₅- is 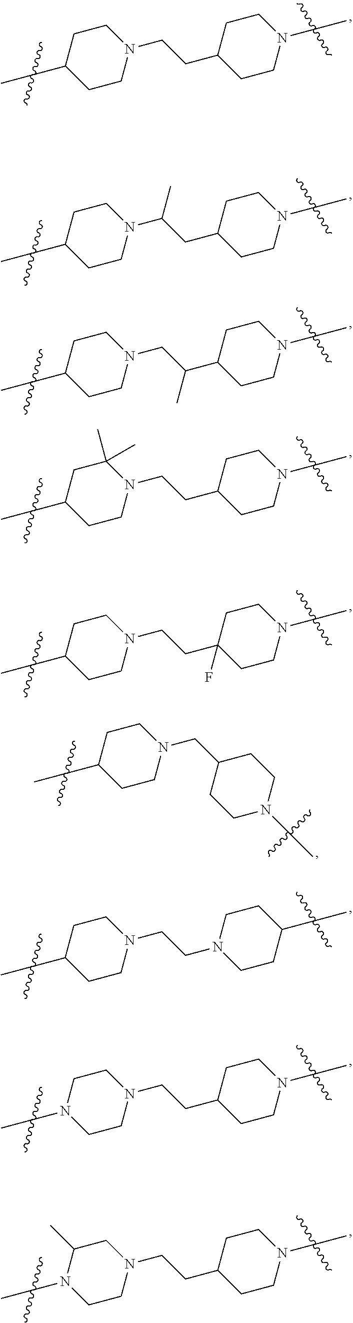 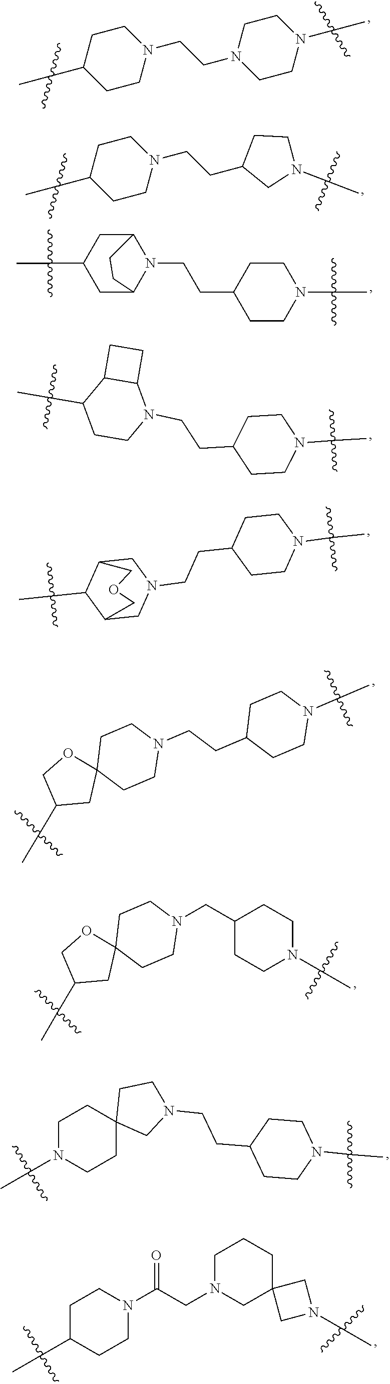 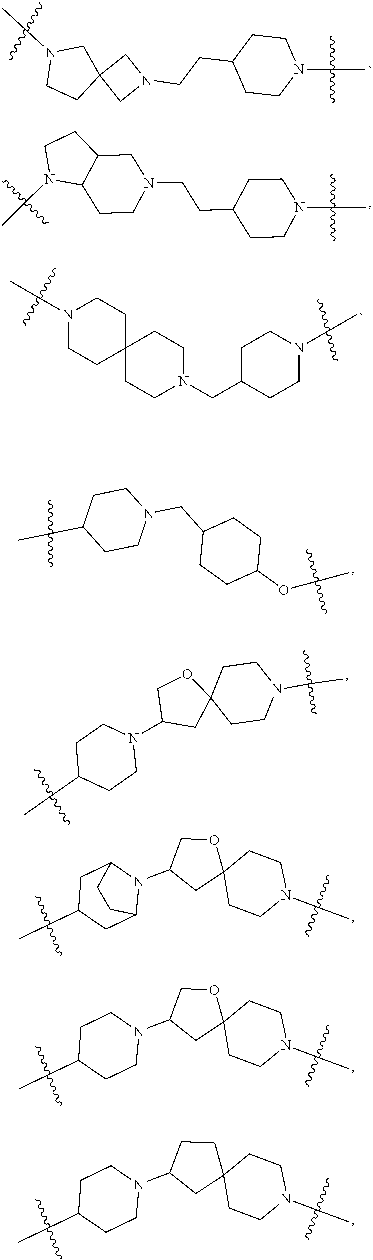 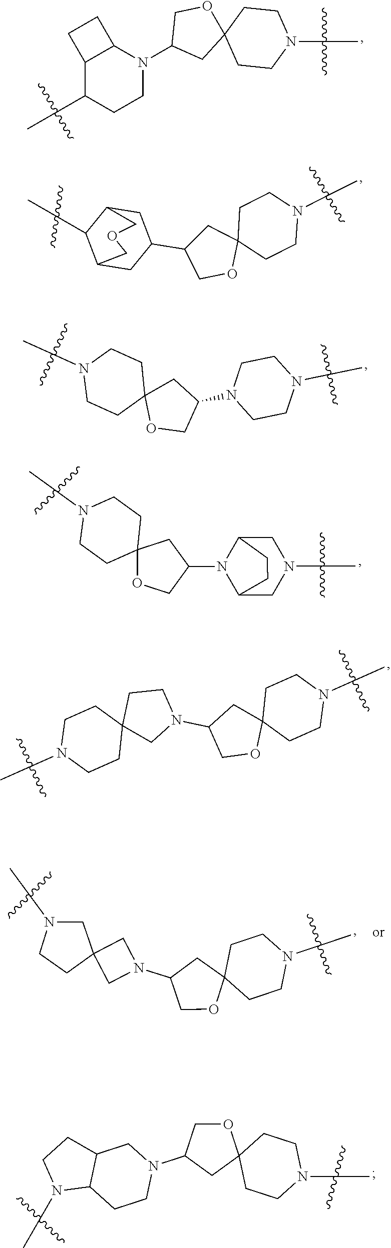
p is 0, 1, or 2; and
R^{j} is halo.

22. The compound of claim 21, wherein R^{c} is C₁₋₆ alkoxy, C₃₋₆ cycloalkyl, C₁₋₆ haloalkyl, or C₁₋₆ alkyl substituted with 1 or 2 C₁₋₃ alkoxy, hydroxy, C₃₋₆ cycloalkyl, phenyl or amino.

23. The compound of claim 21, wherein R³ is OR^{e}, and R^{e} is methyl, ethyl, or cyclopropyl.

24. The compound of claim 21, wherein R³ is pyrazol-5yl (optionally substituted with 1 or 2 methyl, trifluoromethyl, oxan-4-yl), thiazolyl, or indazolyl (optionally substituted with 1 or 2 methyl), —CH₂OCH₃, or O-cyclobutyl.

25. The compound of claim 14, wherein R¹ is hydrogen, R² is hydrogen, L₆, L₇ and L₈ are direct bonds, and W is N, the compound has the following structure: 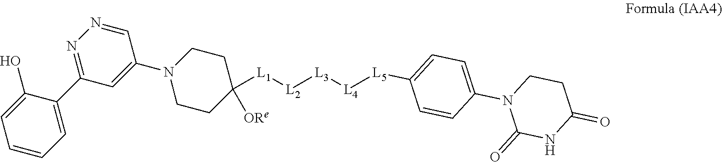
wherein,
R^{e} is C₁₋₆ alkyl;
L₁ is —C(O)—N(R^{c})—, wherein R^{e} is hydrogen, or C₁₋₆ alkyl;
-L₂-L₃-L₄-L₅- is

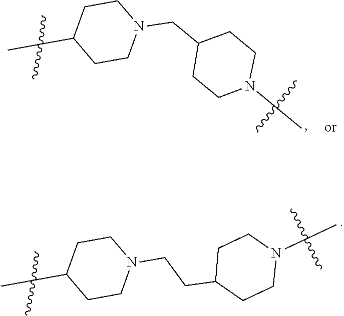 [SMILES: *C1CCN(CC2CCN(*)CC2)CC1 ; *C1CCN(CCC2CCN(*)CC2)CC1]

26. The compound of claim 14, wherein R¹ is hydrogen, R² is hydrogen, L₆, L₇ and L₈ are direct bonds, the compound has the following structure: 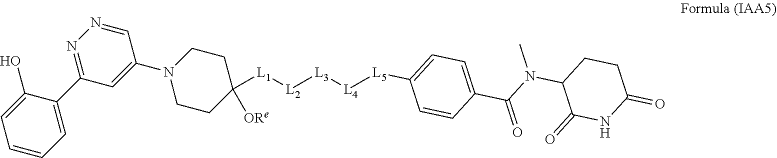
wherein,
R^{e} is C₁₋₆ alkyl;
L₁ is —C(O)—N(R^{c})—, wherein R^{c} is hydrogen, or C₁₋₆ alkyl;
-L₂-L₃-L₄-L₅- is

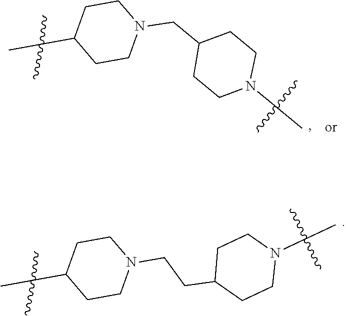 [SMILES: *C1CCN(CC2CCN(*)CC2)CC1 ; *C1CCN(CCC2CCN(*)CC2)CC1]

27. The compound of claim 1, wherein R¹ is hydrogen, L₆, L₇ and L₈ are direct bonds, and the compound has the structure represented by Formula (IAA1): 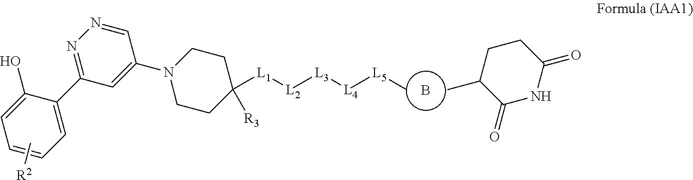
wherein,
R² is hydrogen, halo or C₁₋₃ alkyl;
R³ is halo, C₃₋₆ cycloalkyl, C₆₋₁₂ aryl, C₁₋₆ haloalkyl, C₁₋₆ alkyl optionally substituted with C₁₋₃ alkoxy, or —O—R^{e} (wherein R^{e} is C₃₋₆ cycloalkyl or C₁₋₆ alkyl optionally substituted with one or more C₁₋₃ alkoxy or halo)
L₁ is —C(O)— or —C(O)—N(R^{c})—, wherein R^{c} is hydrogen, C₁₋₆ alkyl, C₁₋₆ alkoxy, C₃₋₆ cycloalkyl (optionally substituted with 1 or 2 halo, C₁₋₃ alkyl or C₁₋₃ alkoxy), 4-12 membered heterocyclyl optionally substituted with 1 or 2 C₁₋₃ alkyl), 5-12 membered heteroaryl optionally substituted with 1 or 2 C₁₋₃ alkyl or C₁₋₃ haloalkyl), phenyl, or C₁₋₆ haloalkyl, wherein C₁₋₆ alkyl is optionally substituted with 1 or 2 C₁₋₃ alkoxy, CN, 5-12 member heteroaryl (optionally further substituted with 1 or 2 halo or C₁₋₃ alkyl), or C₃₋₆ cycloalkyl (optionally further substituted with 1 or 2 halo, C₁₋₃ alkoxy or C₁₋₃ alkyl);
L₂ is 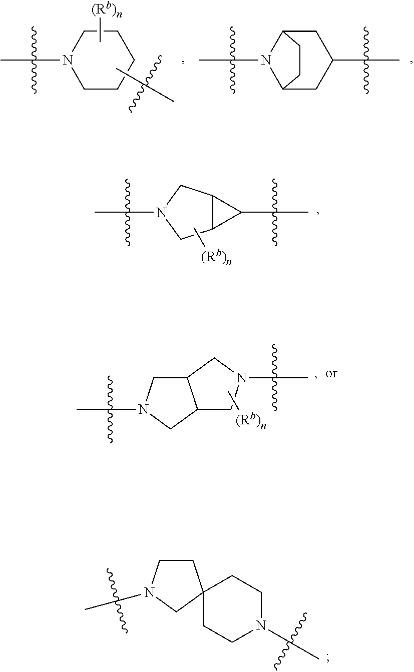
L₃ is direct bond, —CH₂—C(O)—, —(CH₂)₂—O—, C₁₋₄ alkylene chain optionally substituted with C₁₋₃ alkyl or C₂₋₄ alkylene chain optionally substituted with C₁₋₃ alkyl;
L₄ is 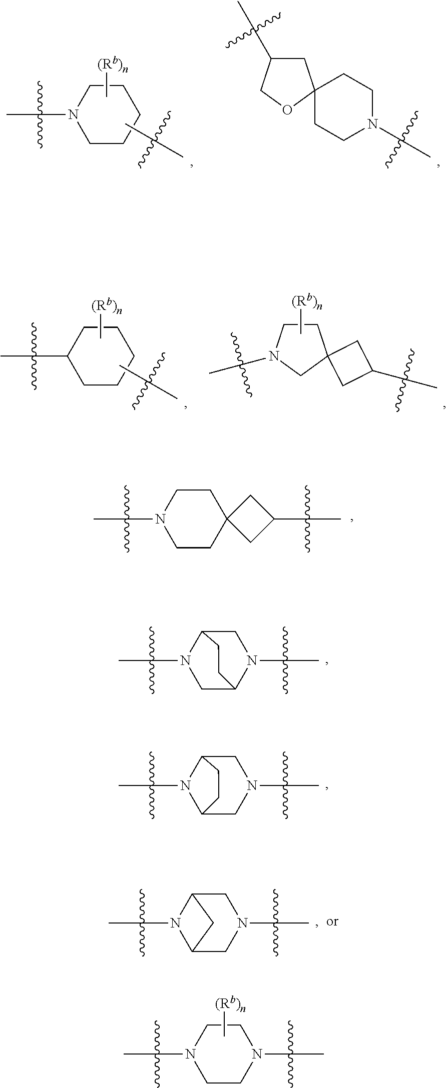
n is 0, 1 or 2, R^{b} is halo, C₁₋₃ alkyl or C₁₋₃ alkoxy;
L₅ is direct bond or —O—;
B is 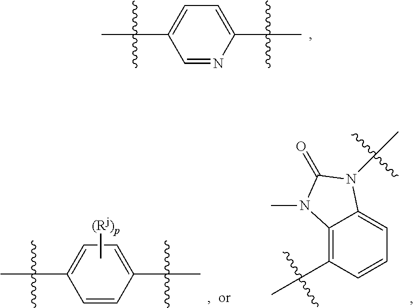
wherein p is 0, 1, or 2, R^{j} is halo.

28. The compound of claim 27, wherein,
R² is hydrogen, F, Cl or methyl;
R³ is F, cyclopropyl, phenyl, benzyl, C₁₋₃ haloalkyl, C₁₋₆ alkyl (optionally substituted 1 or 2 C₁₋₃ alkoxy), or —O—R^{e} (wherein R^{e} is cyclopropyl, cyclobutyl, or C₁₋₆ alkyl optionally substituted with one or more C₁₋₃ alkoxy or halo);
L₁ is —C(O)—N(R^{c})—, wherein R^{c} is C₁₋₆ alkyl, C₁₋₆ alkoxy, cyclopropyl (optionally substituted with methyl or methoxy), cyclobutyl (optionally substituted with 1 or 2 methyl or F), azetidinyl (optionally substituted with 1 or 2 methyl), pyrazolyl (optionally substituted with difluoromethyl), phenyl, or C₁₋₆ haloalkyl, wherein C₁₋₆ alkyl is optionally substituted with 1 or 2 C₁₋₃ alkoxy, CN, oxazolyl, thiazolyl (optionally substituted with methyl), pyrazinyl, pyrimidinyl, 1-methyl-1,2,3-triazolyl, pyrazolyl (optionally substituted with 1 or 2 C₁₋₃ alkyl), oxadiazolyl (optionally substituted with methyl), cyclopropyl (optionally substituted with C₁₋₃ alkoxy), cyclobutyl, or 3,3-difluorocyclobutyl);
-L₂-L₃-L₄-L₅- is

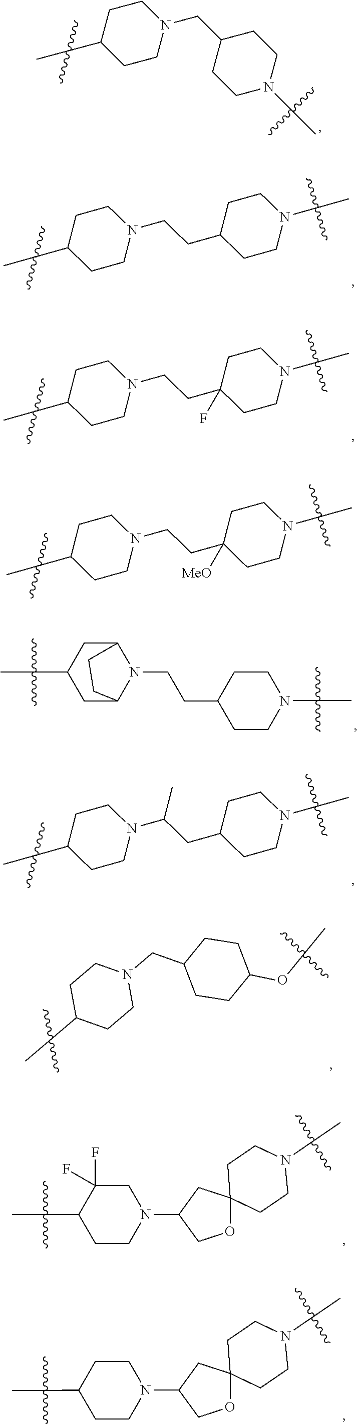

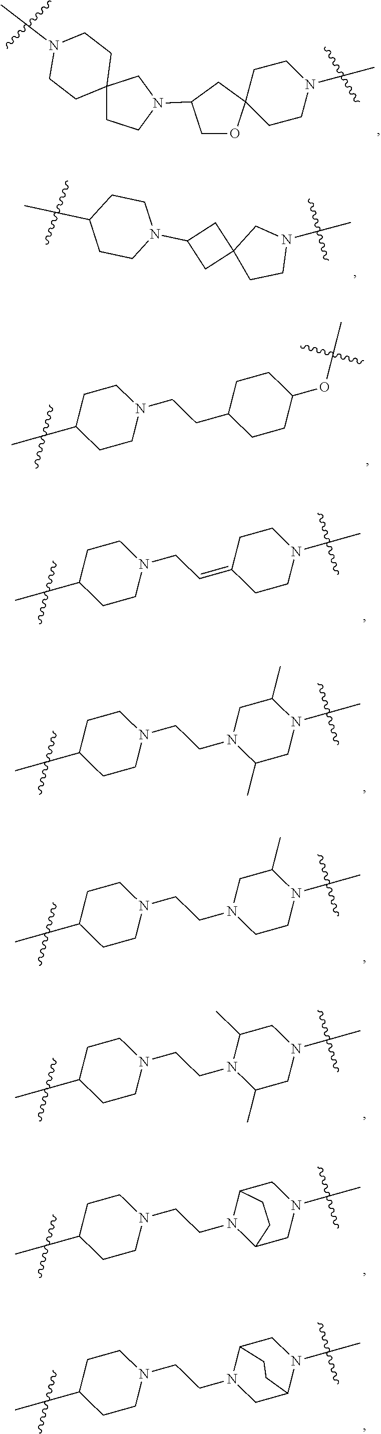

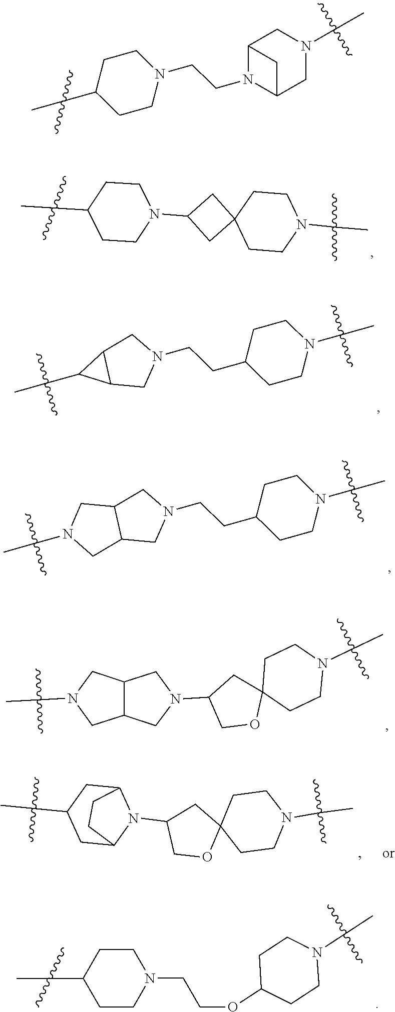

29. The compound of claim 27, wherein,
R² is hydrogen or F;
R³ is C₁₋₆ alkyl (optionally substituted 1 or 2 C₁₋₃ alkoxy), or —O—R^{e} (wherein R^{e} is C₁₋₆ alkyl optionally substituted with one or more C₁₋₃ alkoxy or halo);
L₁ is —C(O)—; and
-L₂-L₃-L₄-L₅- is

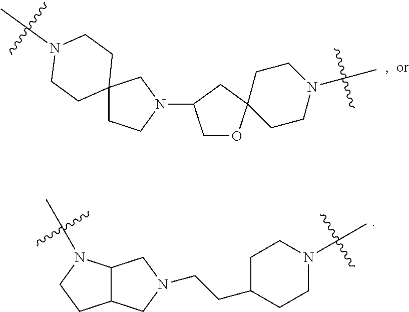 [SMILES: *N1CCC2(CC1)CCN(C1COC3(CCN(*)CC3)C1)C2 ; *N1CCC(CCN2CC3CCN(*)C3C2)CC1]

30. The compound of claim 2, having the following structure: 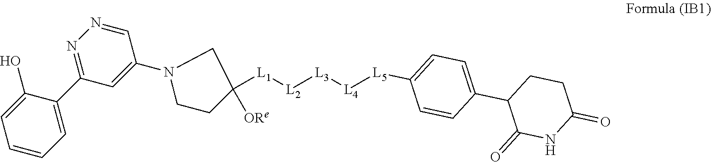
wherein,
R^{e} is C₁₋₆ alkyl;
L₁ is —C(O)—N(R^{c})—, wherein R^{c} is hydrogen, or C₁₋₆ alkyl;
-L₂-L₃-L₄-L₅- is

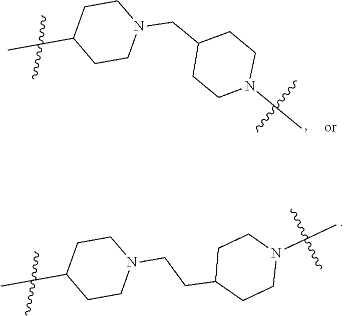 [SMILES: *C1CCN(CC2CCN(*)CC2)CC1 ; *C1CCN(CCC2CCN(*)CC2)CC1]

31. The compound of claim 1, wherein R¹ is hydrogen, L₆, L₇ and L₈ are direct bonds, and the compound has the following structure: 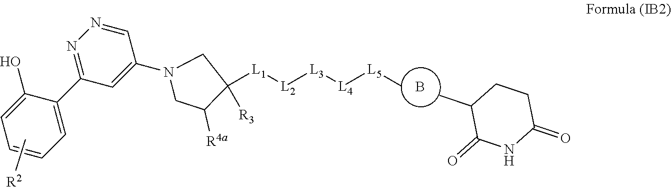
wherein,
R² is hydrogen or halo;
R³ is hydrogen, halo, C₆₋₁₂ aryl, C₁₋₆ alkyl optionally substituted with C₁₋₃ alkoxy, or —O—R^{e} (wherein R^{e} is C₃₋₆ cycloalkyl, or C₁₋₆ alkyl optionally substituted with one or more C₁₋₃ alkoxy or halo);
R^{4a} is hydrogen, C₁₋₆ alkyl or C₁₋₆ alkoxy;
L₁ is —C(O)—, —C(O)—N(R^{c})— or —CH₂—C(O)—N(R^{c})—, wherein R^{c} is hydrogen, C₁₋₆ haloalkyl, C₃₋₆ cycloalkyl (optionally substituted with 1 or 2 halo, C₁₋₃ alkyl or C₁₋₃ alkoxy), or C₁₋₆ alkyl (optionally substituted with 1 or 2 C₁₋₃ alkoxy or 5-12 membered heteroaryl (optionally further substituted with 1 or 2 halo or C₁₋₃ alkyl));
L₂ is 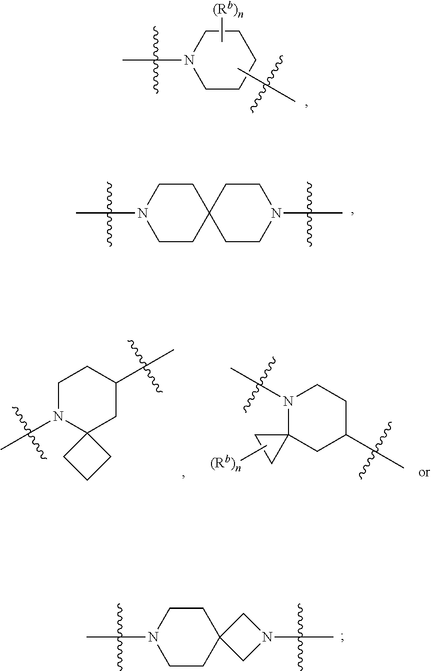
L₃ is direct bond, C₁₋₄ alkylene chain optionally substituted with C₁₋₃ alkyl or C₂₋₄ alkylene chain optionally substituted with C₁₋₃ alkyl;
L₄ is 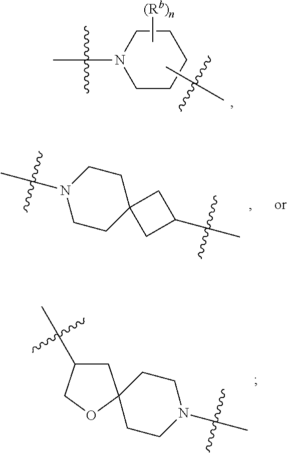
n is 0, 1 or 2, R^{b} is halo, C₁₋₃ alkyl, or hydroxy;
L₅ is direct bond or —O—;
B is 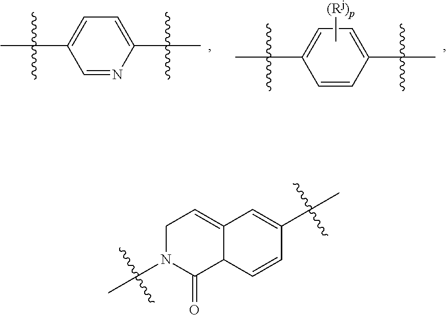
wherein p is 0, 1, or 2, R^{j} is halo.

32. The compound of claim 31, wherein,
R² is hydrogen or F;
R³ is F, C₁₋₃ alkyl, phenyl, or —O—R^{e} (wherein R^{e} is cyclopropyl or C₁₋₃ alkyl);
R^{4a} is hydrogen, or methoxy;
L₁ is —C(O)— or —C(O)—N(R^{c})—, wherein R^{e} is hydrogen, C₁₋₆ alkyl, C₁₋₃ alkyl (optionally substituted with methoxy, oxazolyl, thiazolyl, pyrazinyl, pyrimidinyl, 1-methyl-1,2,3-triazolyl, pyrazolyl (optionally substituted with 1 or 2 C₁₋₃ alkyl), oxadiazolyl (optionally substituted with methyl), or cyclopropyl (optionally substituted with 1 or 2 F, methyl, or methoxy);
-L₂-L₃-L₄-L₅- is

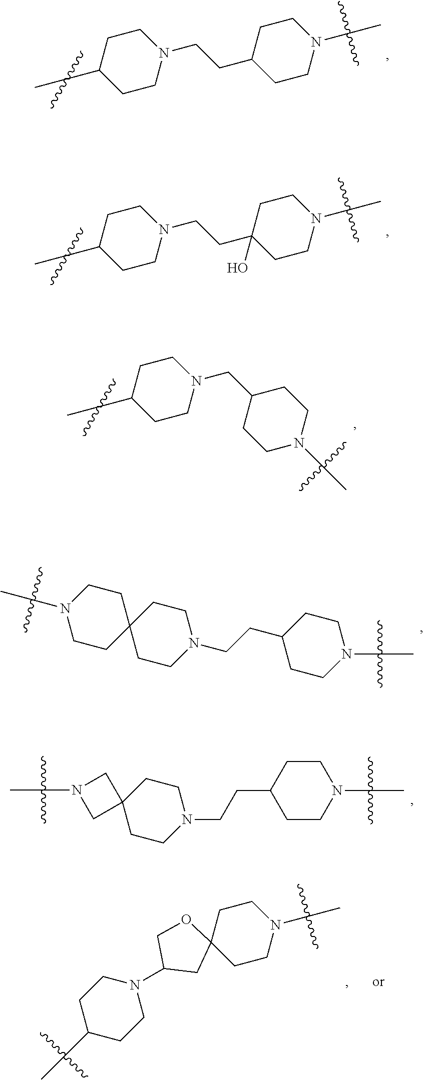 [SMILES: *C1CCN(CCC2CCN(*)CC2)CC1 ; *C1CCN(CCC2(O)CCN(*)CC2)CC1 ; *C1CCN(CC2CCN(*)CC2)CC1 ; *N1CCC(CCN2CCC3(CCN(*)CC3)CC2)CC1 ; *N1CCC(CCN2CCC3(CC2)CN(*)C3)CC1 ; *C1CCN(C2COC3(CCN(*)CC3)C2)CC1]

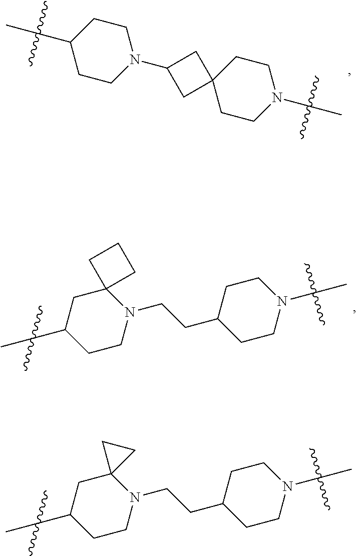 [SMILES: *C1CCN(C2CC3(CCN(*)CC3)C2)CC1 ; *C1CCN(CCC2CCN(*)CC2)C2(CCC2)C1 ; *C1CCN(CCC2CCN(*)CC2)C2(CC2)C1]

33. The compound of claim 1, wherein the compound has one of the following structures: 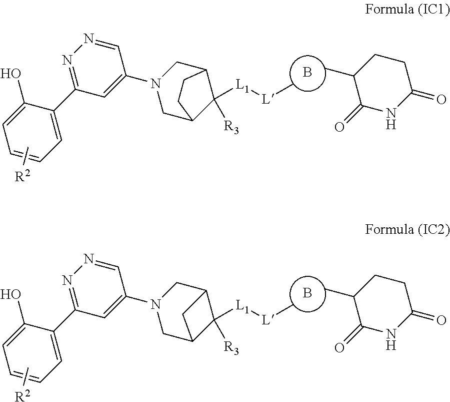
wherein,
R² is hydrogen or halo;
R³ is hydrogen or C₁₋₃ alkyl;
L₁ is —C(O)—N(R^{c})—, wherein R^{c} is hydrogen, C₁₋₆ haloalkyl, C₁₋₆ alkoxy, C₃₋₆ cycloalkyl or C₁₋₆ alkyl optionally substituted with C₃₋₆ cycloalkyl, 5-7 member heteroaryl (preferably thiazolyl), or C₁₋₆ alkoxy;
L′ is 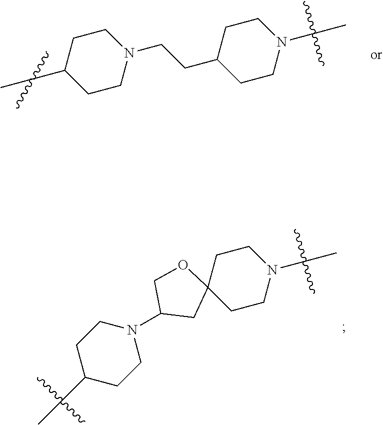 [SMILES: *C1CCN(CCC2CCN(*)CC2)CC1 ; *C1CCN(C2COC3(CCN(*)CC3)C2)CC1]
and
B is 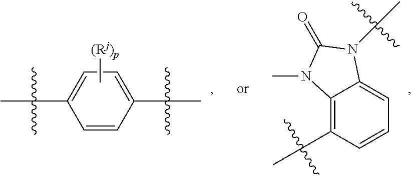
wherein p is 0, 1, or 2, R^{j} is halo.

34. The compound of claim 1, wherein the compound has the following structure: 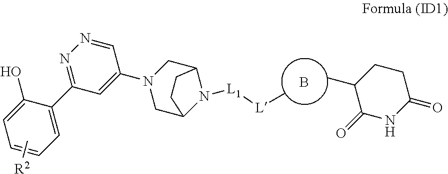
wherein,
R² is hydrogen or halo;
R³ is hydrogen or C₁₋₃ alkyl;
L₁ is —C(O)—; 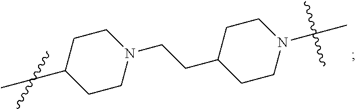 [SMILES: *C1CCN(CCC2CCN(*)CC2)CC1]
L′ is 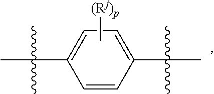
wherein p is 0, 1, or 2, R^{j} is halo.

35. The compound of claim 1, wherein the compound has the following structure: 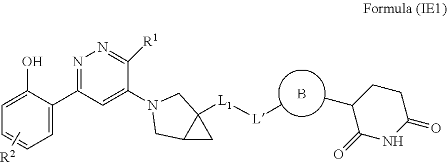
wherein,
R² is hydrogen or halo;
L₁ is —C(O)—N(R^{c})—, wherein R^{c} is hydrogen or C₁₋₆ alkyl;
L′ is 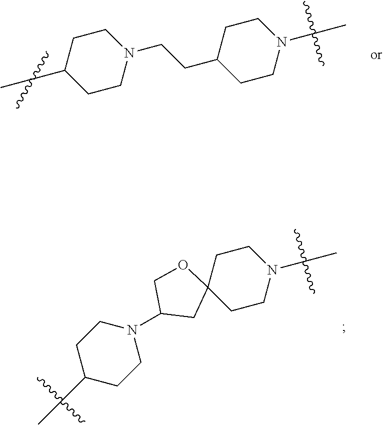 [SMILES: *C1CCN(CCC2CCN(*)CC2)CC1 ; *C1CCN(C2COC3(CCN(*)CC3)C2)CC1]
and
B is 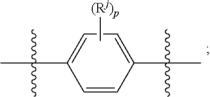
wherein p is 0, 1, or 2, R^{j} is halo.

36. The compound of claim 1, wherein the compound has the following structure: 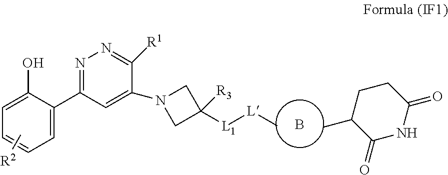
wherein,
R² is hydrogen or halo;
R³ is hydrogen, C₁₋₆ alkyl, or —OR^{e} (wherein R^{e} is C₁₋₆ alkyl);
L₁ is —C(O)—N(R^{c})—, wherein R^{c} is hydrogen or C₁₋₆ alkyl;
L′ is 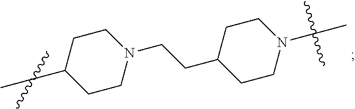 [SMILES: *C1CCN(CCC2CCN(*)CC2)CC1]
and
B is 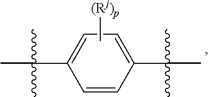
wherein p is 0, 1, or 2, R^{j} is halo.

37. A compound selected from the group consisting of Examples 1-263.

38. A pharmaceutical composition comprising the compound of claim 1, or a pharmaceutically acceptable salt thereof, and a pharmaceutically acceptable excipient or carrier.

39. The pharmaceutical composition of claim 38, further comprising one or more additional therapeutic agents, or a pharmaceutically acceptable salt thereof.

40. (canceled)

41. A method for treating SMARCA2-mediated diseases in a subject in need thereof, the method comprising administering to the subject a therapeutically effective amount of a compound of claim 1.

42. The method according to claim 41 wherein the SMARCA2-mediated diseases are cancers selected from the group consisting of acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, liver cancer, lung cancer, lymphagioendotheliosarcoma, lymphangiosarcoma, lymphoblastic leukemia, lymphoma (Hodgkin's and non-Hodgkin's; Burkitt's), malignancies and hyperproliferative disorders of the bladder, breast, colon, lung, ovaries, pancreas, prostate, skin and uterus, lymphoid malignancies of T-cell or B cell origin, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, malignant rhabdoid tumor (MRT), rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.
